(19) United States
(12) Patent Application Publication
(10) Pub. No.: US 2026/0217677 A1
(43) Pub. Date: Jul. 30, 2026

(54) ORGANIC COMPOUNDS AS NLRP3 INHIBITORS

(71) Applicant: KODIAK SCIENCES, INC., Palo Alto, CA (US)

(72) Inventors: Qianli Meng, Palo Alto, CA (US); Michael Joseph Bishop, San Diego, CA (US); Bernd Jandeleit, Palo Alto, CA (US)

(21) Appl. No.: 19/145,109

(22) PCT Filed: Jan. 2, 2024
(86) PCT No.: PCT/US2024/010081

§ 371 Date: Jul. 1, 2025

Related U.S. Application Data
(60) Provisional application No. 63/478,268, filed on Jan. 3, 2023.
(60) Provisional application No. 63/467,565, filed on May 18, 2023.

(51) Int. Cl. C07D 401/14; A61K 31/501; A61K 31/502; A61K 31/5025; C07D 401/12; C07D 405/14; C07D 409/14; C07D 471/04; C07D 471/10; C07D 487/04; C07D 491/048

(52) U.S. Cl. CPC C07D 401/14; A61K 31/501; A61K 31/502; A61K 31/5025; C07D 401/12; C07D 405/14; C07D 409/14; C07D 471/04; C07D 471/10; C07D 487/04; C07D 491/048

(57) ABSTRACT

Disclosed herein are small molecule compounds that are capable of inhibiting NLRP3 family proteins and NLRP3 inflammasome function in various disease settings. Also disclosed herein also include pharmaceutical compositions, the use and preparations thereof.

### CROSS REFERENCE TO RELATED APPLICATIONS

The present application claims priority to U.S. Provisional Application No. 63/478,268, filed on Jan. 3, 2023, and U.S. Provisional Application No. 63/467,565, filed on May 18, 2023, each incorporated by reference in its entirety.

### BACKGROUND OF THE INVENTION

### Field of the Invention

The present disclosure relates generally to the field of NLRP3 (NOD-like receptor-, LRR- and pyrin domain-containing 3) family proteins and NLRP3 inflammasome function. More specifically, the present disclosure relates to the field of small molecule compounds that are capable of inhibiting NLRP3 family proteins and NLRP3 inflammasome function in various disease settings.

### Description of the Related Art

Inflammasomes are large dynamic multimeric protein complexes formed in the cytosol, upon activation by either pathogen-associated molecular patterns (PAMPs) due to bacterial infection or by endogenous danger signals, danger-associated molecular patterns (DAMPs), released from damaged or dying cells. Tschopp and Martinon in 2002 established a connection between the inflammasomes and the activation of pro-inflammatory protease caspase-1. Caspase-1 is a proteolytic enzyme responsible for converting the inactive precursors, Pro-IL-1ß and Pro-IL-18, to their active form IL-1ß and IL-18 respectively. Both IL-1ß and IL-18 belong to the IL-1 family of cytokines that play key roles in innate and adaptive immune responses impacting a wide range of disease conditions. Tschopp's discovery positioned inflammasomes at the center of immune regulation and homeostasis. Since then, the field has expanded tremendously with new insights garnered on assembly, regulation, and function of inflammasome complexes, as well as their roles in the inflammatory associated diseases.

The inflammasome complexes are composed of three components, a sensor, an adaptor, and an effector enzyme. The sensor is usually a nucleotide-binding domain and leucine-rich-repeat-containing (NLR) protein or an AIM2-like receptor (ALR) protein. The majority of the inflammasomes contain a sensor from the NLR family, and importantly, each inflammasome has its own unique sensor protein. The adaptor protein is called ASC (apoptosis-associated speck-like protein containing a CARD), which recruits and links the effector enzyme to the sensor. ASC is a common component in many inflammasome complexes. Finally, the effector enzyme typically is pro-caspase-1. Upon assembly and activation of the inflammasomes, pro-caspase-1 will be converted to active caspase-1 which will proceed with the activation of key cytokines such as IL-1ß and IL-18.

### SUMMARY OF THE INVENTION

The present disclosure provides a compound represented by the structure of Formula (1-I):

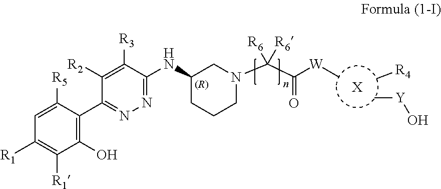

or a pharmaceutically acceptable salt, solvate, isomer, atropisomer, or tautomer thereof, wherein ring

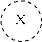

is a 4-11 membered cycloalkyl, heterocycloalkyl, substituted cycloalkyl, or substituted heterocyloalkyl ring; W is selected from the group consisting of NH, N-Me, N-Et and N-iPr, or is a bond; R₁ is selected from the group consisting of —CN, —H, —F, and —CF₃; R₁′ is H; or R₁′ together with R₁ form a thiophene ring; R₂ is selected from the group consisting of H, C1-C3 alkyl, and —CF₃; R₃ is selected from the group consisting of H, C1-C3 alkyl, and —CF₃; or R₃ together with R₂ form a 5- or 6-membered aromatic or non-aromatic ring, all carbon or with O or N within the ring, optionally substituted with -Me or —OH; R₄ is selected from the group consisting of H, —F, C1-C3 alkyl, and —CF₃; R₅ is selected from the group consisting of H, Me, and —CF₃; R₆ and R₆′ are independently selected from the group consisting of —H, —F, C1-C6 alkyl, C1-C6 cycloalkyl, and —CF₃; or R₆ and R₆′ together with the atom they are bonded to form a C1-C6 cycloalkyl or C1-C6 heterocycloalkyl ring; Y is a bond or C1-C3 alkyl optionally substituted with 1, 2, or 3 C1-C3 alkyl or —CF₃; and n is 1, 2, 3, or 4.

In several embodiments, the compound of Formula (1-I) is further represented by any one of the following:

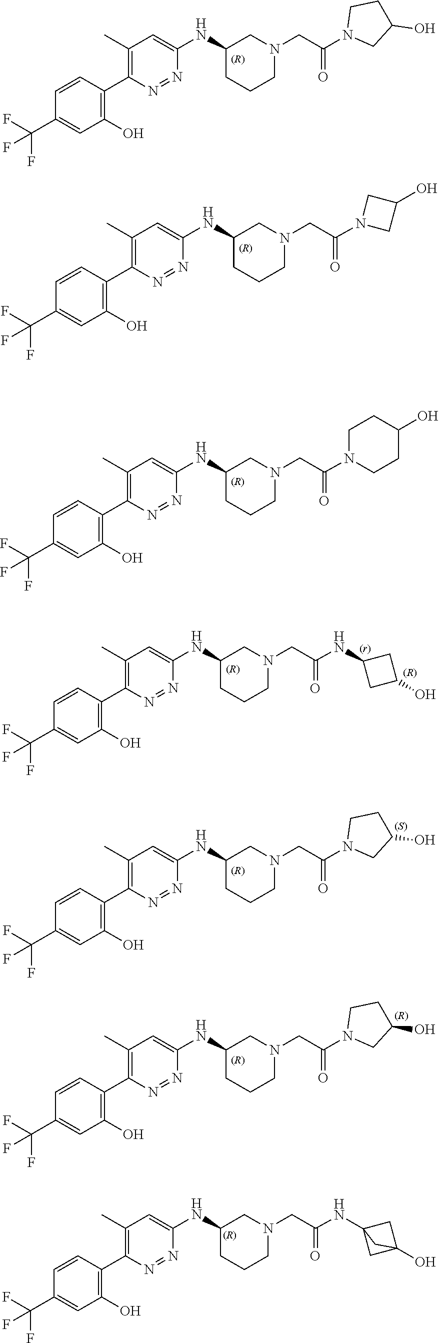

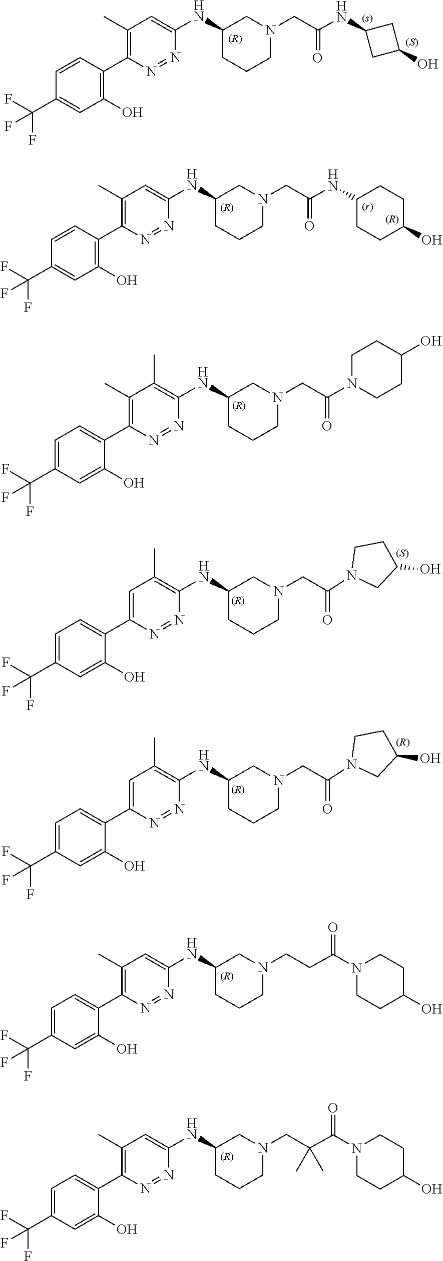

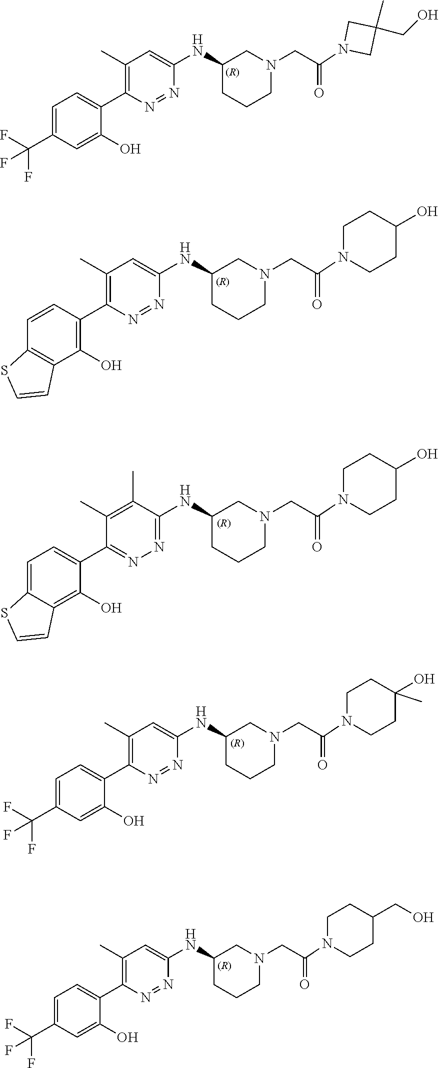

In several embodiments, the compound of Formula (1-I) is further represented by any one of the following:

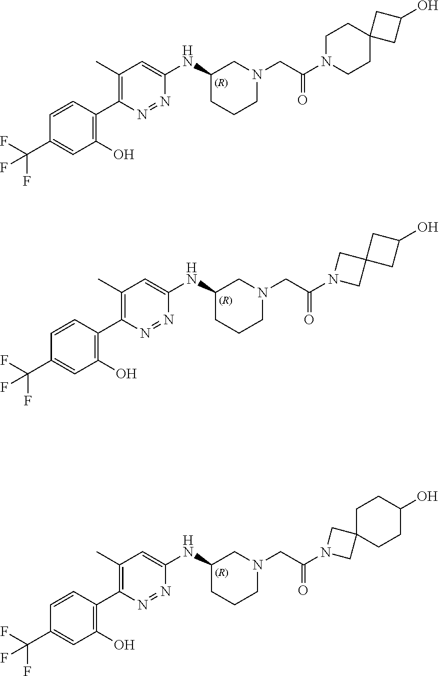

In several embodiments, the compound of Formula (1-I) is further represented by any one of the following:

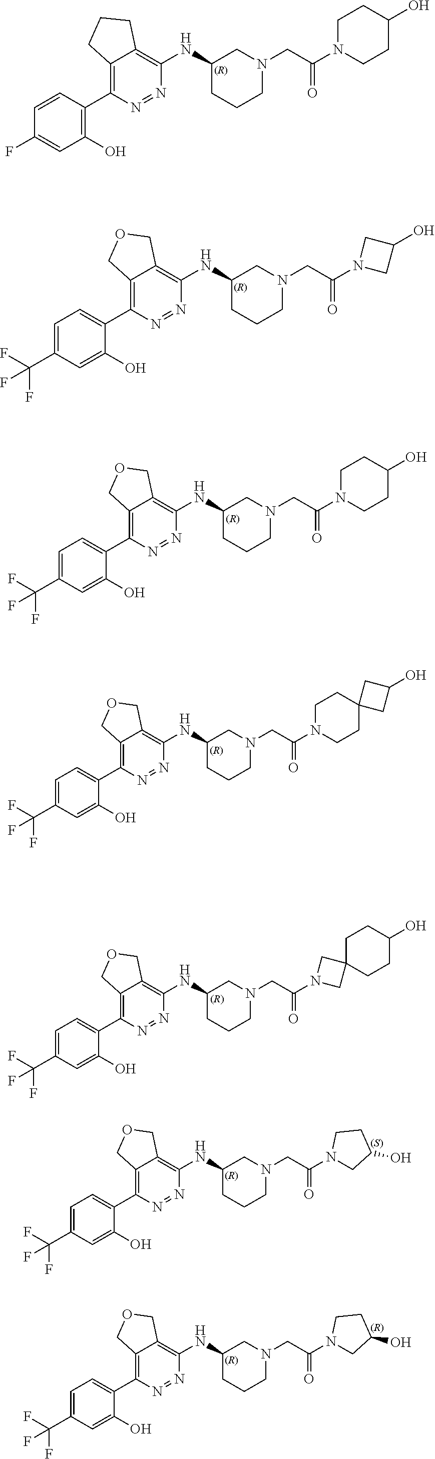

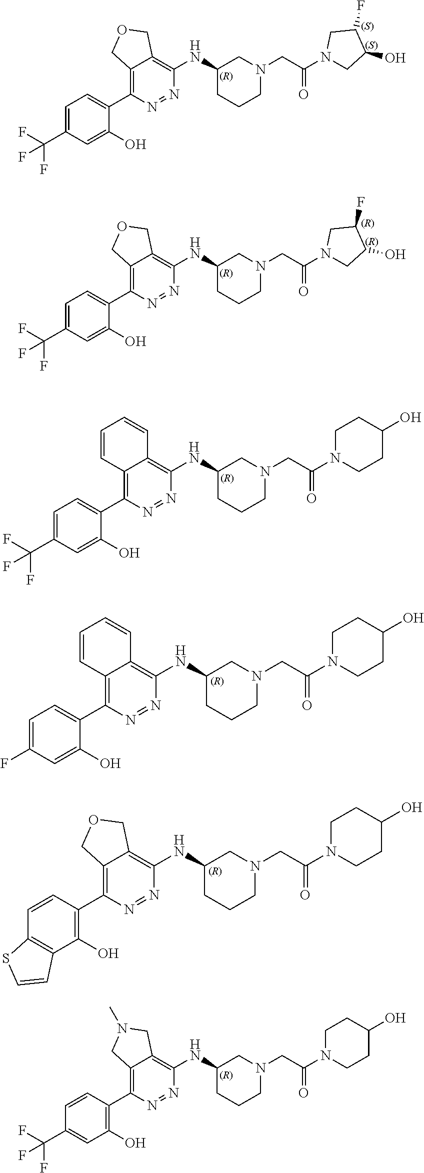

The present disclosure also provides a compound represented by the structure of Formula (1-II):

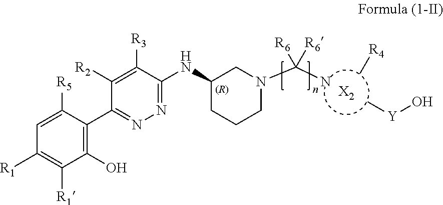

or a pharmaceutically acceptable salt, solvate, isomer, atropisomer, or tautomer thereof; wherein ring

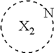

is a 4-11 membered nitrogen-containing heterocycloalkyl ring or substituted heterocycloalkyl ring; R₁ is selected from the group consisting of —CN, —H, —F, and —CF₃; R₁′ is H; or R₁ together with R₁′ form a thiophene ring; R₂ is selected from the group consisting of H, C1-C3 alkyl, and —CF₃; R₃ is selected from the group consisting of H, C1-C3 alkyl, and —CF₃; or R₂ together with R₃ form a 5- or 6-membered aromatic or non-aromatic ring, all carbon or with O or N, optionally substituted with -Me or —OH; Y is a bond or C1-C3 alkyl optionally substituted with 1, 2, or 3 C1-C3 alkyl or —CF₃; R₄ is selected from the group consisting of H, —F, C1-C3 alkyl, and —CF₃; R₅ is selected from the group consisting of H, Me, and —CF₃; R₆ and R₆′ are independently selected from the group consisting of —H, —F, C1-C6 alkyl, C1-C6 cycloalkyl, and —CF₃; or R₆ and R₆′ together with the atom they are bonded to form a C1-C6 cycloalkyl or C1-C6 heterocycloalkyl ring; and n is 2, 3, or 4.

In several embodiments, the compound of Formula (1-II) is further represented by any one of the following:

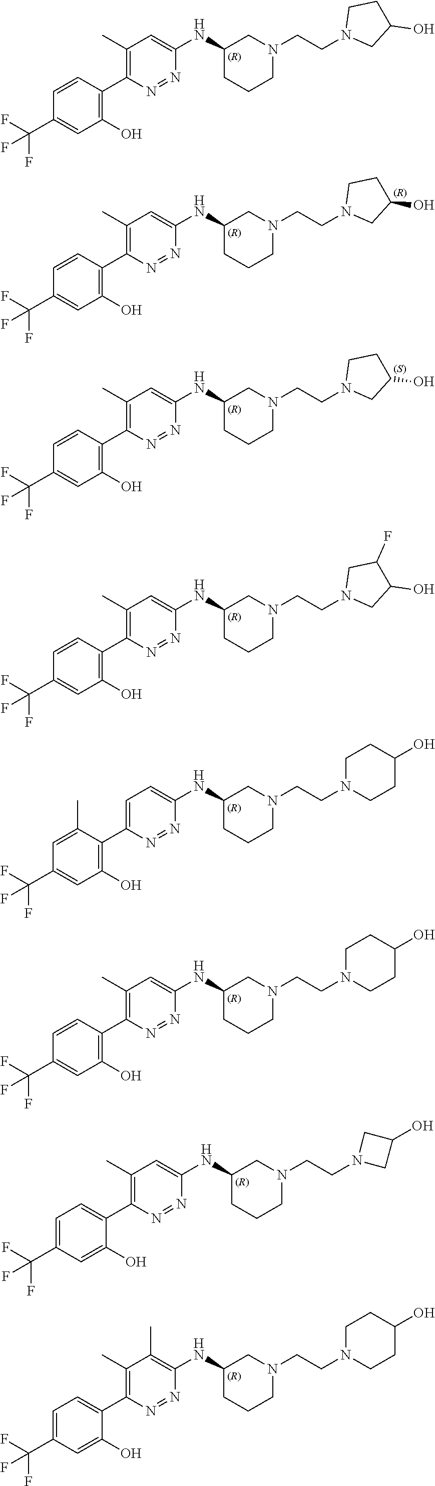

In several embodiments, the compound of Formula (1-II) is further represented by any one of the following:

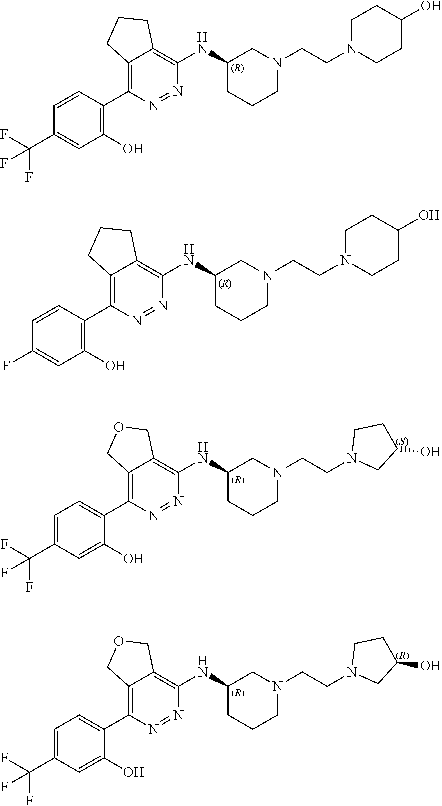

The present disclosure also provides a compound represented by the structure of Formula (1-III):

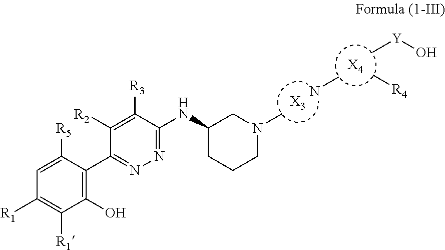

or a pharmaceutically acceptable salt, solvate, isomer, atropisomer, or tautomer thereof, wherein ring

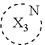

is a 4-7 membered nitrogen-containing heterocycloalkyl ring or substituted heterocycloalkyl ring; ring

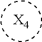

is a 4-7 membered cycloalkyl ring or substituted cycloalkyl ring; R₁ is selected from the group consisting of —CN, —H, —F, and —CF₃; R₁′ is H; or R₁ and R₁′ together form a thiophene ring; R₂ is selected from the group consisting of H, C1-C3 alkyl, and —CF₃; R₃ is selected from the group consisting of H, C1-C3 alkyl, and —CF₃; or R₂ together with R₃ form a 5- or 6-membered aromatic or non-aromatic ring, all carbon or with O or N, optionally substituted with -Me or —OH; R₄ is selected from the group consisting of H, —F, C1-C3 alkyl, and —CF₃; and Y is a bond or C1-C3 alkyl optionally substituted with 1, 2, or 3 C1-C3 alkyl or —CF₃.

In several embodiments, the compound of Formula (1-III) is further represented by any one of the following:

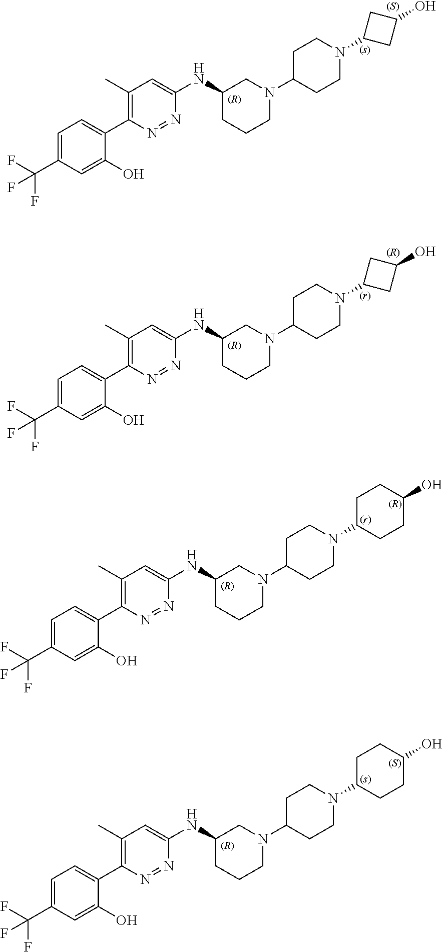

The present disclosure also provides a compound represented by the structure of Formula (1-IV):

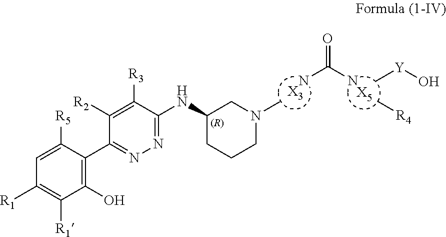

or a pharmaceutically acceptable salt, solvate, isomer, atropisomer, or tautomer thereof, wherein ring

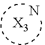

is a 4-7 membered nitrogen-containing heterocycloalkyl ring or substituted heterocycloalkyl ring; wherein ring

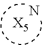

is a 4-7 membered nitrogen-containing heterocycloalkyl ring or substituted hetereocycloalkyl ring; R₁ is selected from the group consisting of —CN, —H, —F, and —CF₃; R₁′ is H; or R₁ and R₁′ together form a thiophene ring; R₂ is selected from the group consisting of H, C1-C3 alkyl, and —CF₃; R₃ is selected from the group consisting of H, C1-C3 alkyl, and —CF₃; or R₂ together with R₃ form a 5- or 6-membered aromatic or non-aromatic ring, all carbon or with O or N within the ring, optionally substituted with -Me or —OH; Y is a bond or C1-C3 alkyl optionally substituted with 1, 2, or 3 C1-C3 alkyl or —CF₃; R₄ is selected from the group consisting of H, —F, C1-C3 alkyl, and —CF₃; and R₅ is selected from the group consisting of —H, -Me and —CF₃.

In several embodiments, the compound of Formula (1-IV) is further represented by any one of the following:

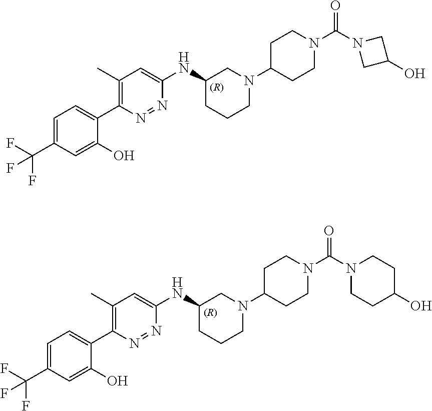

The present disclosure also provides a compound represented by the structure of Formula (1-V):

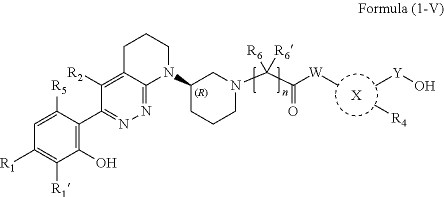

or a pharmaceutically acceptable salt, solvate, isomer, atropisomer, or tautomer thereof, wherein ring

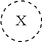

is a 4-11 membered cycloalkyl heterocycloalkyl, substituted cycloalkyl, or substituted heterocycloalkyl ring; W is NH, N-Me, N-Et, N-iPr, or a bond; R₁ is selected from the group consisting of —CN, —H, —F, and —CF₃; R₁′ is H; or R₁ and R₁′ together form a thiophene ring; R₂ is selected from the group consisting of H, C1-C3 alkyl, —CF₃; R₄ is selected from the group consisting of H, —F, C1-C3 alkyl, and —CF₃; Y is a bond or C1-C3 alkyl optionally substituted with 1, 2, or 3 C1-C3 alkyl or —CF₃; R₅ is selected from the group consisting of —H, -Me, and —CF₃; R₆ and R₆′ are independently selected from the group consisting of —H, —F, C1-C6 alkyl, and —CF₃, or R₆ and R₆′ together with the atom they are bonded to form a C1-C6 cycloalkyl or C1-C6 heterocycloalkyl ring; and n is 1, 2, 3, or 4.

In several embodiments, the compound of Formula (1-V) is further represented by any one of the following:

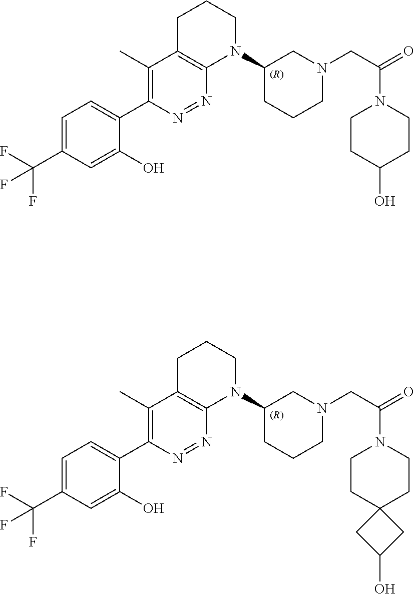

The present disclosure also provides a compound represented by the structure of Formula (1-VI):

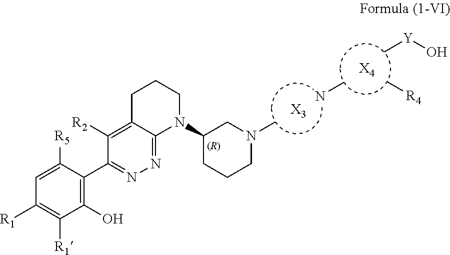

wherein ring

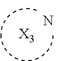

is an optionally substituted 4-7 membered nitrogen-containing heterocycloalkyl ring or substituted heterocycloalkyl ring; ring

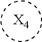

is an optionally substituted 4-7 membered cycloalkyl ring or substituted cycloalkyl ring; R₁ is selected from the group consisting of —CN, —H, —F, and —CF₃; R₁′ is H; or R₁ and R₁′ together form a thiophene ring; R₂ is selected from the group consisting of —H, C1-C3 alkyl, and —CF₃; R₄ is selected from the group consisting of —H, —F, C1-C3 alkyl, and —CF₃; R₅ is selected from the group consisting of —H, -Me, and —CF₃; and Y is a bond or C1-C3 alkyl optionally substituted with 1, 2, or 3 C1-C3 alkyl or —CF₃.

In several embodiments, the compound of Formula (1-VI) is further represented by any one of the following:

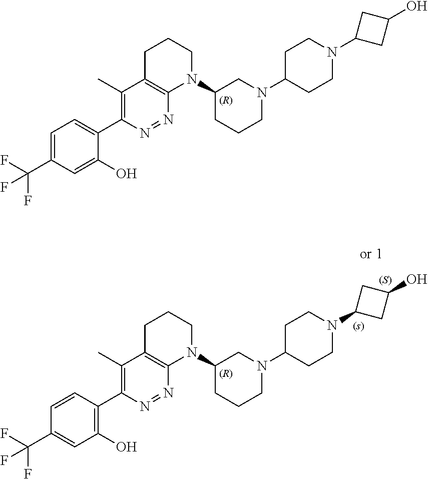

The present disclosure also provides a compound represented by the structure of Formula (1-VII):

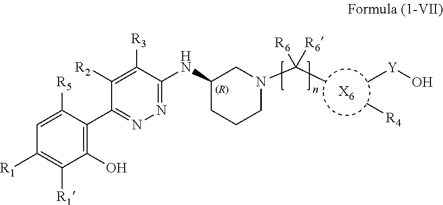

wherein ring

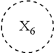

is a 4-11 membered cycloalkyl heterocycloalkyl, substituted cycloalkyl, or substituted heterocycloalkyl ring; R₁ is selected from the group consisting of —CN, —H, —F, and —CF₃; R₁′ is H; or R₁ and R₁′ together form a thiophene ring; R₂ is selected from the group consisting of —H, C1-C3 alkyl, —CF₃; R₄ is selected from the group consisting of —H, —F, C1-C3 alkyl, and —CF₃; R₅ is selected from the group consisting of —H, -Me, and —CF₃; R₆ and R₆′ are independently selected from the group consisting of —H, —F, C1-C6 alkyl, C1-C6 cycloalkyl, and —CF₃, or R₆ and R₆′ together with the atom they are bonded to form a C1-C6 cycloalkyl or C1-C6 heterocycloalkyl ring; n is 0, 1, 2, 3, or 4; and Y is a bond or C1-C3 alkyl optionally substituted with 1, 2, or 3 C1-C3 alkyl or —CF₃.

In several embodiments, the compound of Formula (1-VII) is further represented by any one of the following:

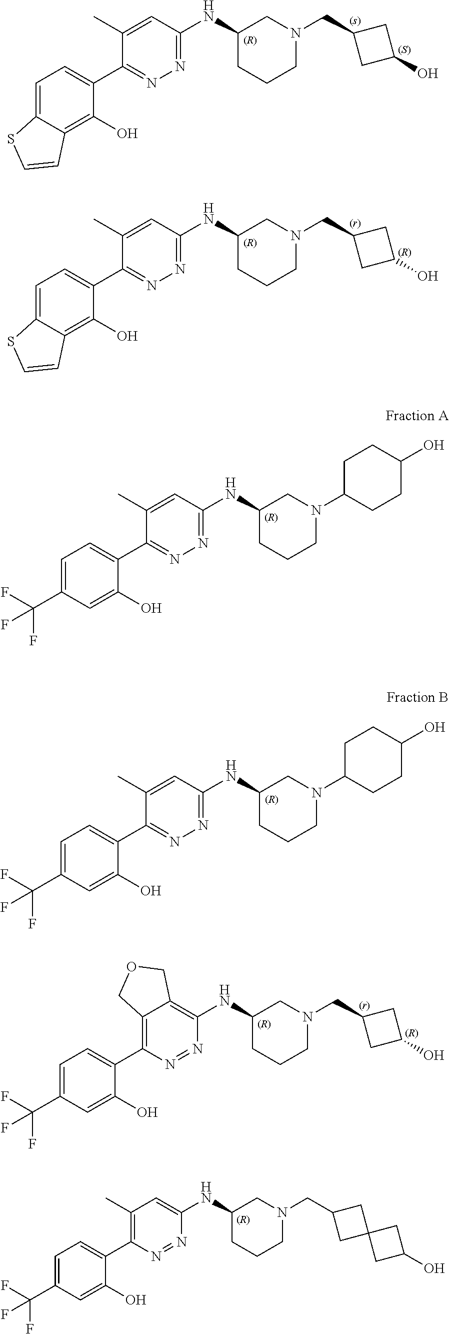

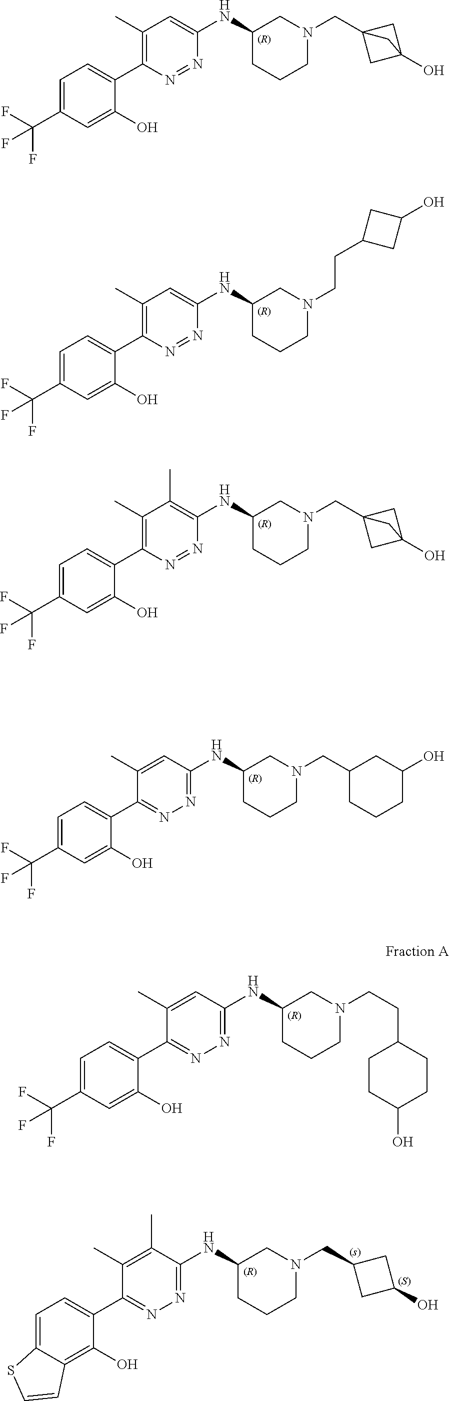

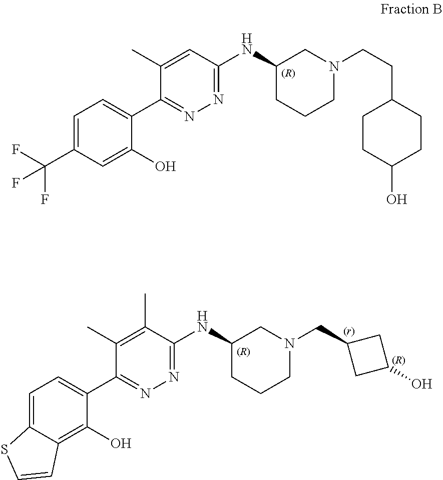

The present disclosure also provides a compound represented by the structure of Formula (1-VIII):

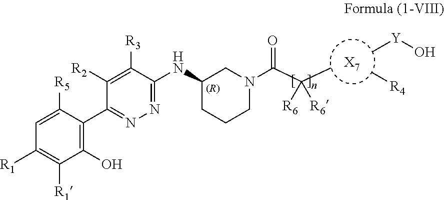

wherein ring

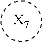

is a 4-11 membered cycloalkyl, heterocycloalkyl, substituted cycloalkyl, or substituted heterocycloalkyl ring; R₁ is selected from the group consisting of —CN, —H, —F, and —CF₃; R₁′ is H; or R₁ and R₁′ together form a thiophene ring; R₂ is selected from the group consisting of —H, C1-C3 alkyl, and —CF₃; R₄ is selected from the group consisting of H, —F, C1-C3 alkyl, and —CF₃; R₅ is selected from the group consisting of H, Me, and —CF₃; R₆ and R₆′ are independently selected from the group consisting of —H, —F, C1-C6 alkyl, and —CF₃, or R₆ and R₆′ together with the atom they are bonded to form a C1-C6 cycloalkyl or C1-C6 heterocycloalkyl ring; n is 1, 2, 3, or 4; and Y is a bond (or C1-C3 alkyl optionally substituted with 1, 2, or 3 C1-C3 alkyl or —CF₃.

In several embodiments, the compound of Formula (1-VIII) is further represented by any one of the following:

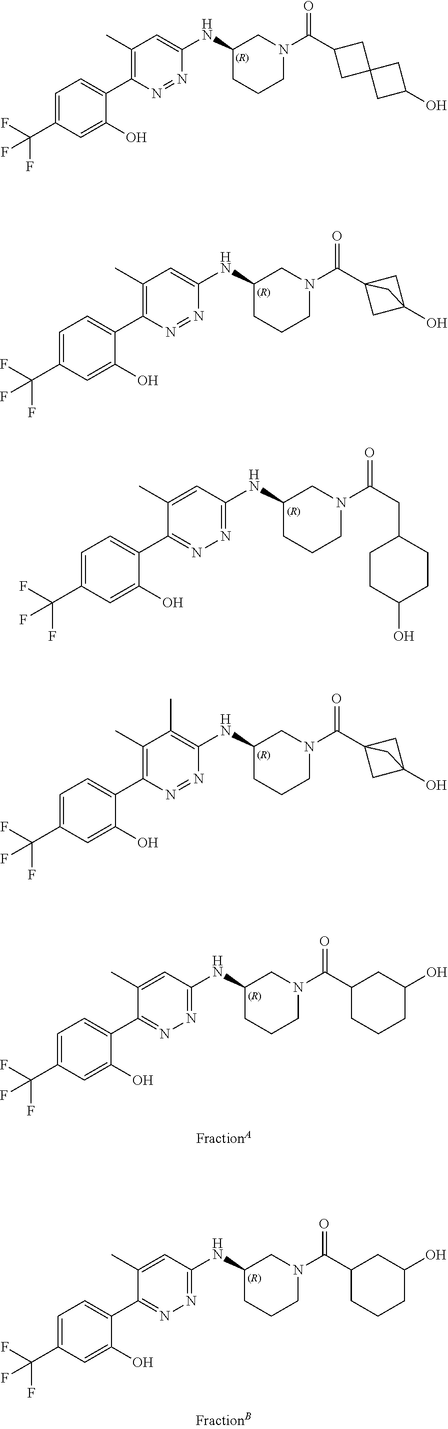

The present disclosure also provides a compound represented by the structure of Formula (1-IX):

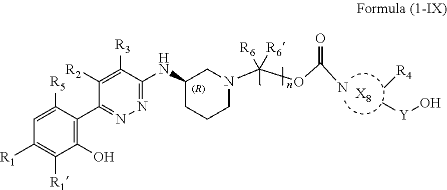

or a pharmaceutically acceptable salt, solvate, isomer, atropisomer, or tautomer thereof, wherein ring

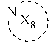

is a 4-11 membered nitrogen-containing heterocycloalkyl ring or substituted heterocycloalkyl ring, R₁ is selected from the group consisting of —CN, —H, —F, and —CF₃; R₁′ is H; or R₁ and R₁′ together form a thiophene ring; R₂ is selected from the group consisting of H, C1-C3 alkyl, —CF₃; R₃ is selected from the group consisting of H, C1-C3 alkyl, and —CF₃; or R₂ together with R₃ form a 5- or 6-membered aromatic or non-aromatic ring, all carbon or with O or N within the ring, optionally substituted with -Me or —OH; R₄ is selected from the group consisting of H, —F, C1-C3 alkyl, and —CF₃; R₅ is selected from the group consisting of H, Me, and —CF₃; R₆ and R₆′ are independently selected from the group consisting of —H, —F, C1-C6 alkyl, C1-6 cycloalkyl, and —CF₃, or R₆ and R₆′ together with the atom they are bonded to form a C1-C6 cycloalkyl or C1-C6 heterocycloalkyl ring; n is 2, 3, or 4; and Y is a bond (or C1-C3 alkyl optionally substituted with 1, 2, or 3 C1-C3 alkyl or —CF₃.

In several embodiments, the compound of Formula (1-IX) is further represented by any one of the following:

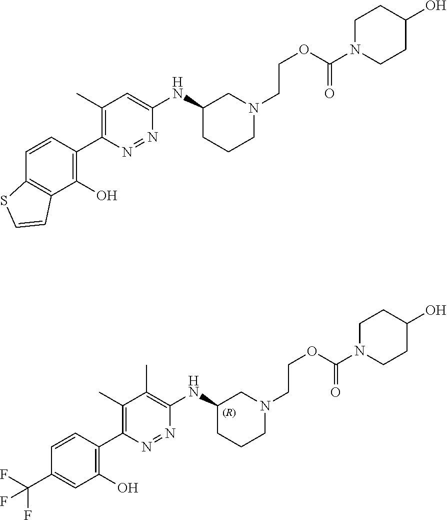

The present disclosure provides a compound represented by the structure of Formula (2-I):

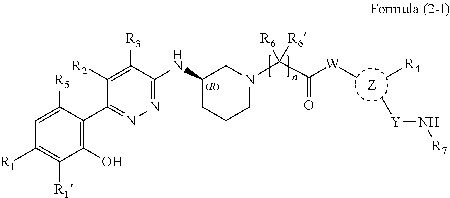

or a pharmaceutically acceptable salt, solvate, hydrate, isomer, atropisomer, or tautomer thereof, wherein ring

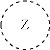

is a 4-11 membered cycloalkyl, substituted cycloalkyl, heterocycloalkyl, or substituted heterocycloalkyl ring; W is selected from the group consisting of NH, N-Me, N-Et and N-iPr, or W is a bond; R₁ is selected from the group consisting of —CN, —H, —F, and —CF₃; R₁′ is —H; or R₁ together with R₁′ form a thiophene ring; each R₂ and R₃ is selected from the group consisting of —H, C1-C3 alkyl, and —CF₃; or R₂ together with R₃ forms a 5- or 6-membered aromatic or non-aromatic ring containing all C or at least one O or N, optionally substituted with -Me or —OH; R₄ is selected from the group consisting —H, —F, C1-C3 alkyl, and —CF₃; Y is a bond or C1-C3 alkyl optionally substituted with 1, 2, or 3 C1-C3 alkyl or —CF₃. R₅ is selected from the group consisting of —H, -Me and —CF₃; R₆ and R₆′ are independently selected from the group consisting of —H, —F, C1-C6 alkyl or cycloalkyl, and —CF₃; or R₆ and R₆′ together with the atom they are bonded to form a C1-C6 cycloalkyl or C1-C6 heterocycloalkyl ring; n is 1, 2, 3, or 4; and R₇ is selected from the group consisting of —H, C1-C6 alkyl, cycloalkyl, heterocylcoalkyl,

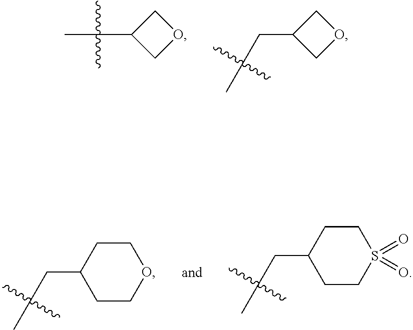 [SMILES: *C1COC1 ; *CC1COC1 ; *CC1CCOCC1 ; *CC1CCS(=O)(=O)CC1]

In some embodiments, R1 is selected from —CN, —H, —F, —CF3; or may form a thiophene ring together with R1′ (to make a benzothiophene with the sulfur atom attached to the carbon atom bearing R1); R1′ is —H or may form a thiophene ring together with R1 (to make a benzothiophene with the sulfur atom attached to the carbon atom bearing R1); R2 is selected from —H, C1-C3 alkyl, —CF3, or it forms a ring together with R3, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R3 is selected from —H, C1-C3 alkyl, —CF3, or it forms a ring together with R2, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R4 is selected from —H, —F, C1-C3 alkyl, and —CF3; Y is a bond (when Y is a bond, the —NH may be attached directly to the ring; similarly, when Y is a bond, the —NH—R7 may be attached directly to the ring) or Y is C1-C3 alkyl, optionally substituted with 1, 2, or 3 C1-C3 alkyl, or —CF3; R5 is selected from —H, -Me and —CF3; R6 and R6′ are independently selected from —H, —F, C1-C6 alkyl or cycloalkyl, and —CF3, or R6 and R6′ together with the atom they are bonded to form a C1-C6 cycloalkyl or C1-C6 heterocycloalkyl ring; n is selected from 1, 2, 3, and 4; R7 is selected from —H, C1-C6 alkyl, cycloalkyl, heterocycloalkyl,

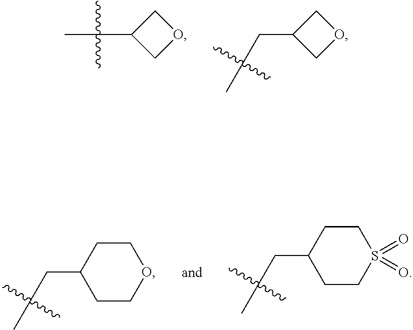 [SMILES: *C1COC1 ; *CC1COC1 ; *CC1CCOCC1 ; *CC1CCS(=O)(=O)CC1]

In some aspects of Formula (2-I), ring

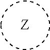

is a 4-11 membered ring, selected from cycloalkyl, substituted cycloalkyl, heterocycloalkyl or substituted heterocycloalkyl, including spirocyclic and bicyclic cycloalkyl, substituted spirocyclic and bicyclic cycloalkyl, spirocyclic and bicyclic heterocycloalkyl, and substituted spirocyclic and bicyclic heterocycloalkyl, and where W is selected from NH, N-Me, or is a bond; when W is a bond, the carbonyl group (C═O) is bonded to a nitrogen atom within ring

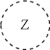

so as together they form an amide bond; R₁ is selected from —F, —CF₃; R₁′ is —H; R₂ is selected from —H, -Me, —CF₃, or it forms a ring together with R₃, a 5- or 6-membered non-aromatic ring, all carbon or with O within the ring; R₃ is selected from —H, -Me, or it forms a ring together with R₂, a 5- or 6-membered non-aromatic ring, all carbon or with O within the ring; R₄ is —H; R₅ is selected from —H and -Me; R₆ and R₆′ are —H; n is 1; R₇ is —H.

In several embodiments, the compound of Formula (2-I) is further represented by any one of the following:

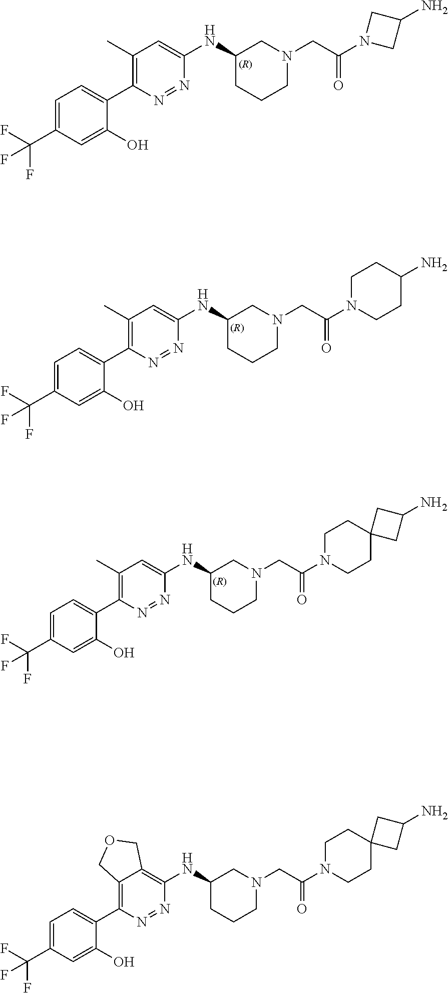

The present disclosure also provides a compound represented by the structure of Formula (2-II):

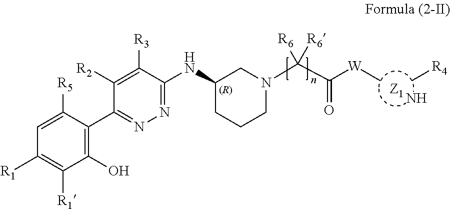

or a pharmaceutically acceptable salt, solvate, hydrate, isomer, atropisomer, or tautomer thereof, wherein ring

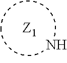

is a 4-11 membered nitrogen-containing heterocycloalkyl ring or substituted heterocycloalkyl ring; W is selected from NH, N-Me, N-Et and N-iPr, or is a bond; R₁ is selected from the group consisting of —CN, —H, —F, and —CF₃; R₁′ is H; or R₁ and R₁′ together form a thiophene ring; R₂ and R₃ are each independently selected from the group consisting of H, C1-C3 alkyl, and —CF₃; or R₂ together with R₃ form a 5- or 6-membered aromatic or non-aromatic ring, all carbon or with O or N within the ring, optionally substituted with -Me or —OH; R₄ is selected from the group consisting of —H, —F, C1-C3 alkyl, and —CF₃; R₅ is selected from the group consisting of —H, -Me, and —CF₃; R₆ and R₆′ are independently selected from the group consisting of —H, —F, C1-C6 alkyl or cycloalkyl, and —CF₃; or R₆ and R₆′ together with the atom they are bonded to form a C1-C6 cycloalkyl or C1-C6 heterocycloalkyl ring; and n is 1, 2, 3, or 4.

In some aspects of Formula (2-II), ring

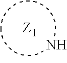

is a 4-11 membered nitrogen-containing heterocycloalkyl ring, including spirocyclic and bicyclic nitrogen-containing heterocycloalkyl, and where W is selected from NH, N-Me, or is a bond; when W is a bond, the carbonyl group (C═O) is bonded to a nitrogen atom within ring

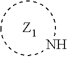

so as together they form an amide bond R₁ is selected from —F, —CF₃; R₁′ is —H; R₂ is selected from —H, -Me, —CF₃, or it forms a ring together with R₃, a 5- or 6-membered non-aromatic ring, all carbon or with O within the ring; R₃ is selected from —H, -Me, or it forms a ring together with R₂, a 5- or 6-membered non-aromatic ring, all carbon or with O within the ring; R₄ is —H or -Me; R₅ is selected from —H and -Me; R₆ and R₆′ are —H; n is 1.

In several embodiments, the compound of Formula (2-II) is further represented by any one of the following:

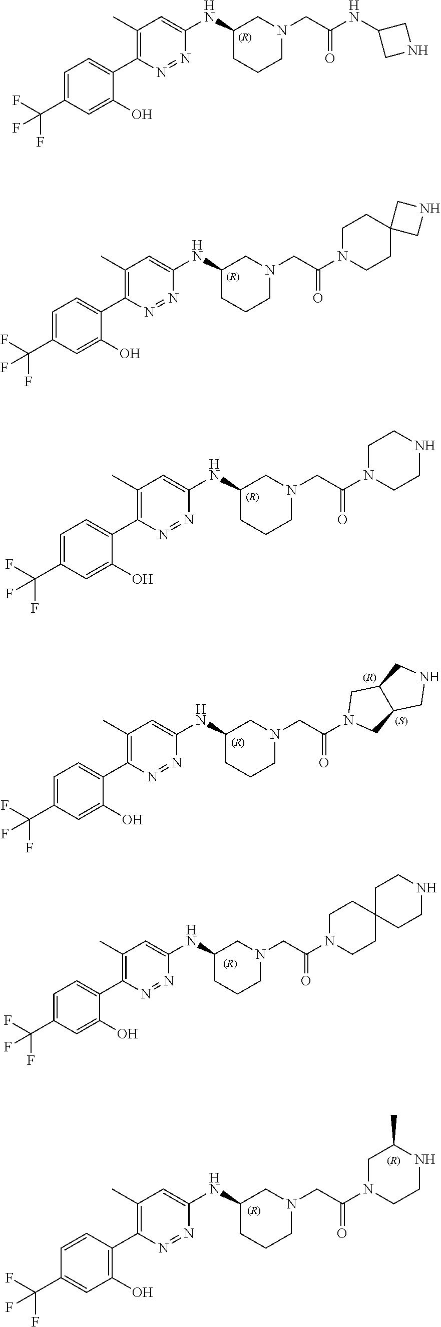

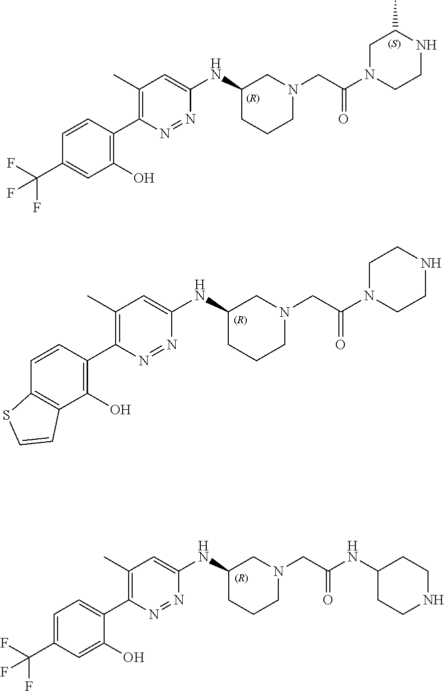

The present disclosure also provides a compound represented by the structure of

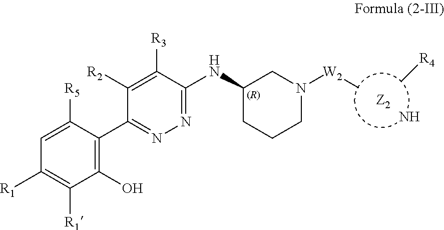

or a pharmaceutically acceptable salt, solvate, hydrate isomer, atropisomer, or tautomer thereof, wherein ring

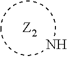

is a 4-11 membered heterocycloalkyl or substituted heterocycloalkyl ring; R₁ is selected from the group consisting of —CN, —H, —F, and —CF₃; R₁′ is H; or R₁ and R₁′ together form a thiophene ring; R₂ is selected from the group consisting of H, C1-C3 alkyl, —CF₃; R₃ is selected from the group consisting of H, C1-C3 alkyl, —CF₃; Or R₂ together with R₃ form a 5- or 6-membered aromatic or non-aromatic ring, all carbon or with O or N within the ring, optionally substituted with -Me or —OH; R₄ is selected from the group consisting of —H, —F, C1-C3 alkyl, and —CF₃; R₅ is selected from the group consisting of —H, -Me and —CF₃; W₂ is selected from the group consisting of optionally substituted —CH₂—, —CH₂CH₂—, and —CH₂CH₂CH₂—, or is a bond.

In some aspects of Formula (2-III), ring

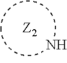

is a 4-9 membered nitrogen-containing heterocycloalkyl ring, including spirocyclic and bicyclic nitrogen-containing heterocycloalkyl, and where W₂ is selected from —CH₂—, —CH₂CH₂—, or is a bond; R₁ is selected from —F, —CF₃; R₁′ is —H; R₂ is selected from —H, -Me, —CF₃, or it forms a ring with R₃, a 5- or 6-membered non-aromatic ring, all carbon or with O within the ring; R₃ is selected from —H, -Me, or it forms a ring with R₂, a 5- or 6-membered non-aromatic ring, all carbon or with O within the ring; R₄ is —H or -Me; R₅ is selected from —H and -Me;

In several embodiments, the compound of Formula (2-III) is further represented by any one of the following:

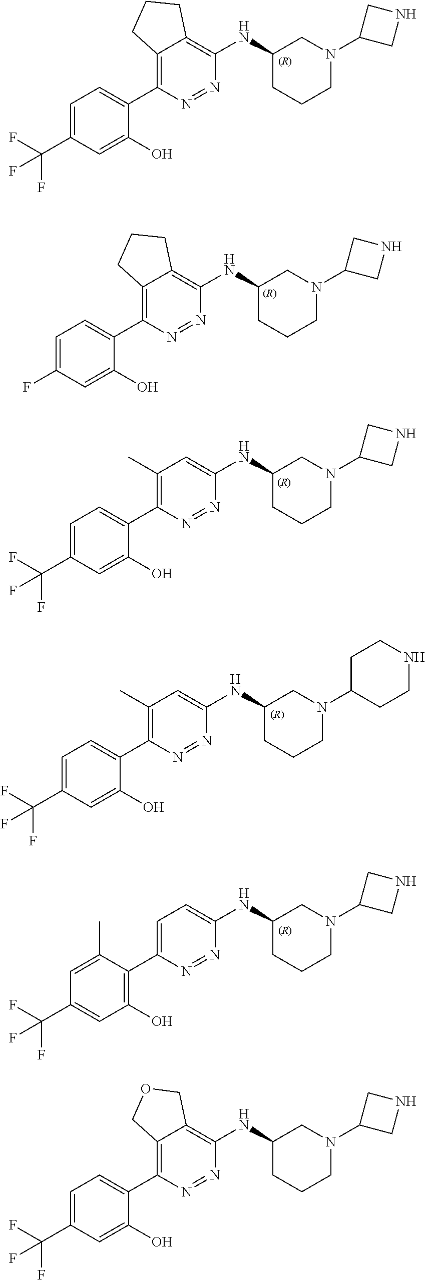

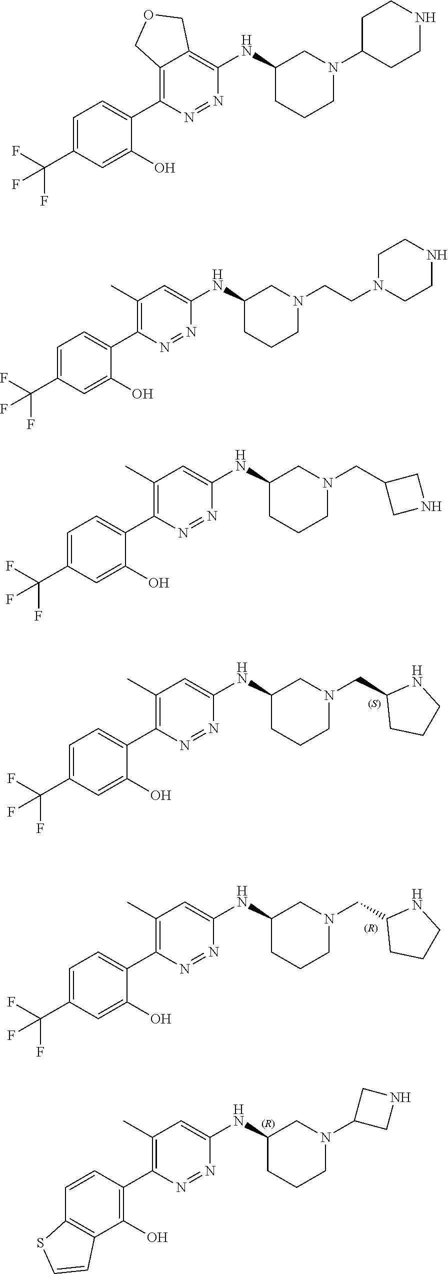

The present disclosure also provides a compound represented by the structure of Formula (2-IV):

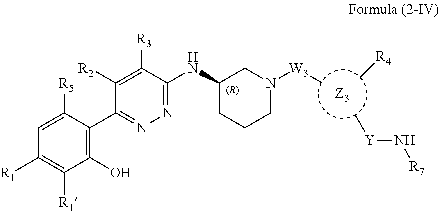

or a pharmaceutically acceptable salt, solvate, hydrate, isomer, atropisomer, or tautomer thereof, wherein: ring

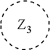

is a 4-11 membered cycloalkyl, substituted cycloalkyl, heterocycloalkyl or substituted heterocycloalkyl ring; R₁ is selected from the group consisting of —CN, —H, —F, and —CF₃; R₁′ is H; or R₁ and R₁′ together form a thiophene ring; R₂ is selected from the group consisting of H, C1-C3 alkyl, —CF₃; R₃ is selected from the group consisting of H, C1-C3 alkyl, —CF₃; or R₂ together with R₃ form a 5- or 6-membered aromatic or non-aromatic ring, all carbon or with O or N within the ring, optionally substituted with -Me or —OH; R₄ is selected from the group consisting of —H, —F, C1-C3 alkyl, and —CF₃; R₅ is selected from the group consisting of —H, -Me, and —CF₃; W₃ is selected from the group consisting of optionally substituted —CH₂—, —CH₂CH₂—, and —CH₂CH₂CH₂—, or is a bond; Y is a bond or C1-C3 alkyl optionally substituted with 1, 2, or 3 C1-C3 alkyl or —CF₃; and R₇ is selected from the group consisting of —H, C1-C6 alkyl, cycloalkyl, heterocycloalkyl,

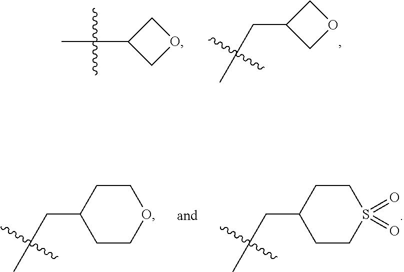 [SMILES: *C1COC1 ; *CC1COC1 ; *CC1CCOCC1 ; *CC1CCS(=O)(=O)CC1]

In some aspects of Formula (2-IV), ring

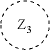

is a 4-9 membered cycloalkyl ring or heterocycloalkyl ring, and where W₃ is selected from —CH₂— and —CH₂CH₂—, or is a bond; R₁ is selected from —F and —CF₃; R₁′ is —H; R₂ is selected from —H, -Me, and —CF₃, or it forms a ring with R₃, a 5- or 6-membered aromatic or non-aromatic ring, all carbon or with O within the ring; R₃ is selected from —H and -Me, or it forms a ring with R₂, a 5- or 6-membered aromatic or non-aromatic ring, all carbon or with O within the ring; R₄ is —H or -Me; R₅ is selected from —H and -Me; R₇ is —H.

In several embodiments, the compound of Formula (2-IV) is further represented by any one of the following:

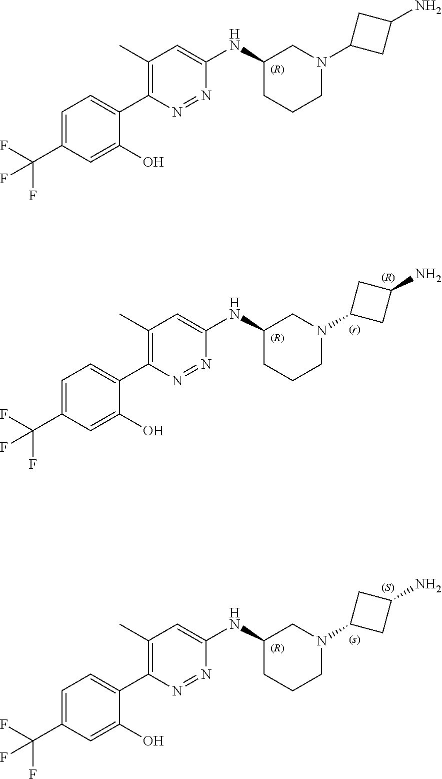

The present disclosure also provides a compound represented by the structure of Formula (2-V):

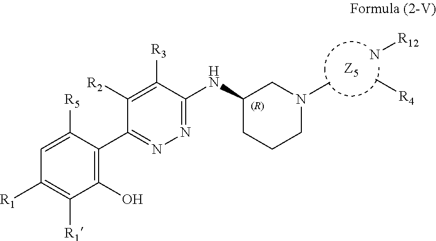

or a pharmaceutically acceptable salt, solvate, isomer, atropisomer or tautomer thereof, wherein: ring

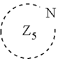

is azetidine, pyrrolidine, piperidine, or azepane; R₁ is selected from the group consisting of —CN, —H, —F, and —CF₃; R₁′ is H; or R₁ and R₁′ together form a thiophene ring; R₂ is selected from the group consisting of H, C1-C3 alkyl, —CF₃; R₃ is selected from the group consisting of H, C1-C3 alkyl, —CF₃; or R₂ together with R₃ form a 5- or 6-membered aromatic or non-aromatic ring, all carbon or with O or N within the ring, optionally substituted with -Me or —OH; R₄ is selected from the group consisting of —H, —F, C1-C3 alkyl, and —CF₃; R₅ is selected from the group consisting of —H, -Me and —CF₃; and R₁₂ is selected from the group consisting of:

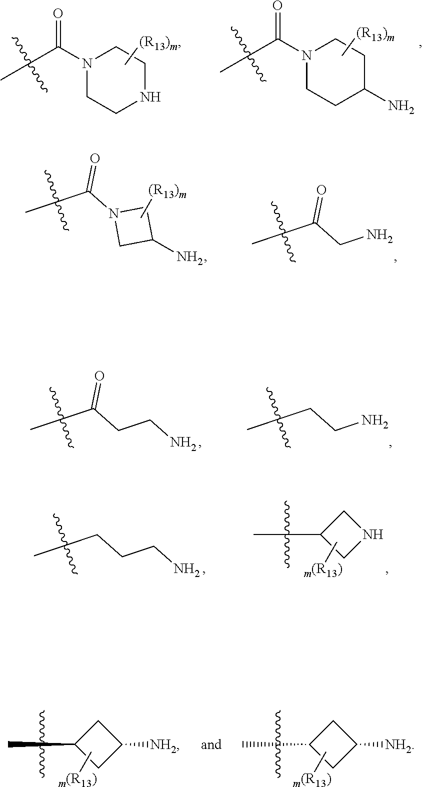

wherein m is 1, 2, or 3 and each R₁₃ is independently selected from the group consisting of —H, C1-C3 alkyl, and —CF; or two R₁₃ groups connect to form a cyclopropyl or cyclobutyl ring.

Rings may have 1, 2, or 3 R₁₃ groups, with each R₁₃ independently selected from —H, C1-C3 alkyl, and —CF₃. Two R₁₃ groups may connect to form a cyclopropyl or cyclobutyl ring.

In some aspects of Formula (2-V), ring

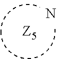

is azetidine or piperidine; R₁ is selected from —F, —CF₃; R₁′ is —H; R₂ is selected from —H, -Me, and —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₄ is —H or -Me; R₅ is selected from —H and -Me; R₁₂ is selected from:

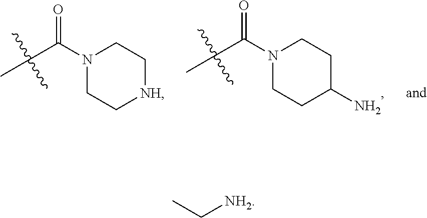

In several embodiments, the compound of Formula (2-V) is further represented by any one of the following:

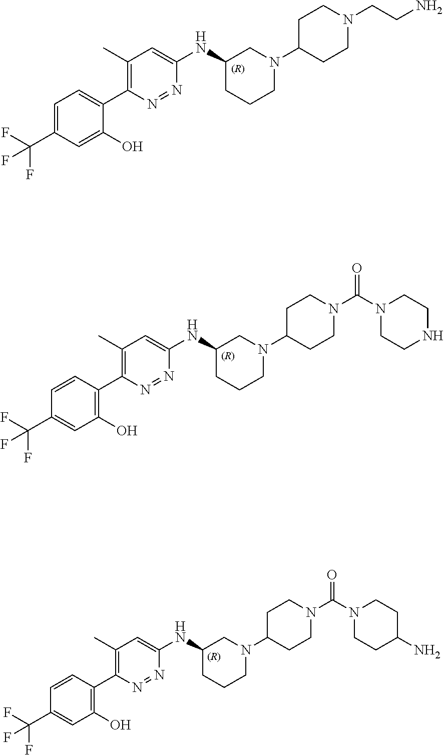

The present disclosure also provides a compound represented by the structure of Formula (2-VI):

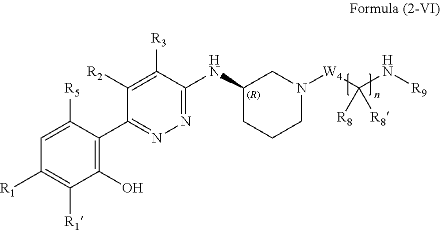

or a pharmaceutically acceptable salt, solvate, hydrate, isomer, atropisomer, or tautomer thereof, wherein R₁ is selected from the group consisting of —CN, —H, —F, and —CF₃; R₁′ is H; or R₁ and R₁′ together form a thiophene ring; R₂ is selected from the group consisting of H, C1-C3 alkyl, and —CF₃; R₃ is selected from the group consisting of H, C1-C3 alkyl, and —CF₃; or R₂ together with R₃ form a 5- or 6-membered aromatic or non-aromatic ring, all carbon or with O or N within the ring, optionally substituted with -Me or —OH; R₅ is selected from the group consisting of —H, -Me and —CF₃; W₄ is selected from the group consisting of —CH₂—, —CH₂CH₂—,

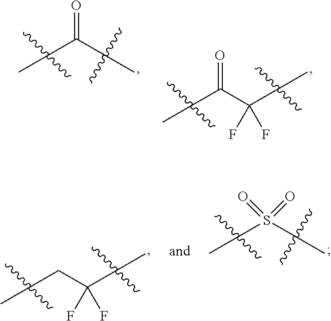

R₈ and R₈′ are independently selected from the group consisting of —H, C1-C5 alkyl, C1-C5 cycloalkyl, and —CF₃; and R₉ is selected from the group consisting of —H, C1-C6 alkyl, C1-C6 cycloalkyl, heterocycloalkyl,

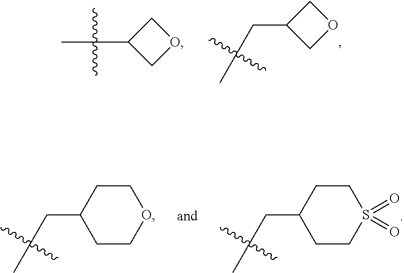 [SMILES: *C1COC1 ; *CC1COC1 ; *CC1CCOCC1 ; *CC1CCS(=O)(=O)CC1]

In some aspects of Formula (2-VI), R₁ is selected from —F, —CF₃; R₁′ is —H; R₂ is selected from —H, -Me, —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₅ is selected from —H and -Me; W₄ is selected from —CH₂— or

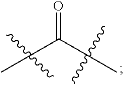 [SMILES: *C(*)=O]

R₈ and R₈′ are —H; n is 1; R₉ is —H.

In some aspects of Formula (2-VI), R₁ is selected from —F, —CF₃; R₁′ is —H; R₂ is selected from —H, -Me, —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₅ is selected from —H and -Me; W₄ is selected from —CH₂—or

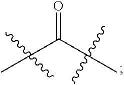 [SMILES: *C(*)=O]

R₈ and R₈′ are —H; n is 1; R₉ is selected from

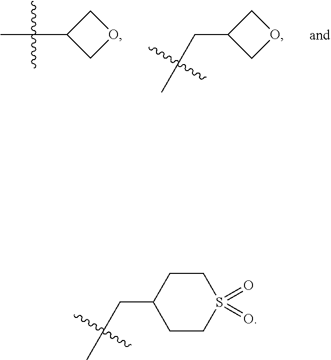 [SMILES: *C1COC1 ; *CC1COC1 ; *CC1CCS(=O)(=O)CC1]

In several embodiments, the compound of Formula (2-VI) is further represented by any one of the following:

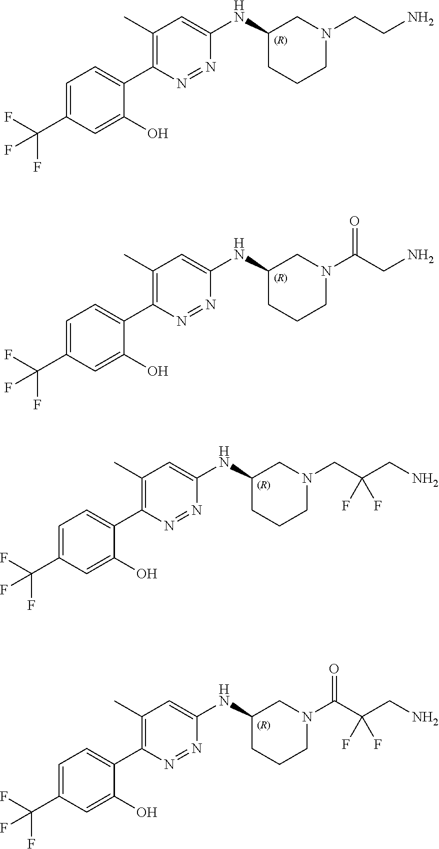

In some embodiments, the compound of Formula (2-VI) is further represented by any one of the following:

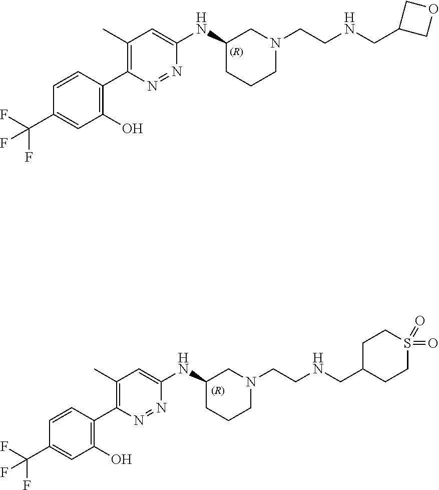

The present disclosure also provides a compound represented by the structure of Formula (2-VII):

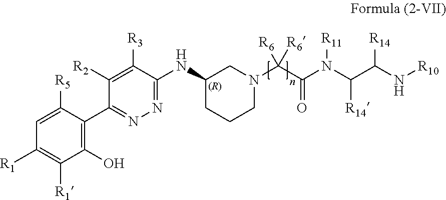

or a pharmaceutically acceptable salt, solvate, hydrate, isomer, atropisomer, or tautomer thereof, wherein: R₁ is selected from the group consisting of —CN, —H, —F, and —CF₃; R₁′ is H; or R₁ and R₁′ together form a thiophene ring; R₂ is selected from the group consisting of H, C1-C3 alkyl, and —CF₃; R₃ is selected from the group consisting of H, C1-C3 alkyl, and —CF₃; or R₂ together with R₃ form a 5- or 6-membered aromatic or non-aromatic ring, all carbon or with O or N within the ring, optionally substituted with -Me or —OH; R₅ is selected from—the group consisting of H, -Me and —CF₃; R₆ and R₆′ are independently selected from the group consisting of —H, —F, C1-C6 alkyl, C1-C6 cycloalkyl, and —CF₃, or R₆ and R₆′ together with the atom they are bonded to form a C1-C6 cycloalkyl or C1-C6 heterocycloalkyl ring; n is 1, 2, 3, or 4; R₁₄ and R₁₄′ are independently selected from the group consisting of H, C1-C5 alkyl (including cycloalkyl), and —CF₃; or R₁₄ and R₁₄′ together with the atoms that they are bonded to form a optionally substituted C3-C6 cycloalkyl or optionally substituted C3-C6 heterocycloalkyl ring; R₁₀ is selected from the group consisting of —H, C1-C7 alkyl, C1-C7 cycloalkyl, C1-C7 methylcycloalkyl),

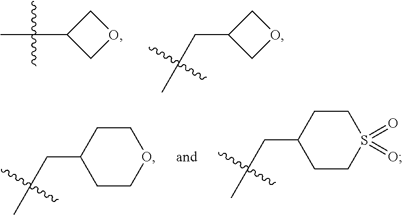 [SMILES: *C1COC1 ; *CC1COC1 ; *CC1CCOCC1 ; *CC1CCS(=O)(=O)CC1]

and R₁₁ is selected from the group consisting of H, C1-C7 alkyl, C1-C7 cycloalkyl, C1-C7 methylcycloalkyl.

In some aspects of Formula (2-VII), R₁ is selected from —F, —CF₃; R₁′ is —H; R₂ is selected from —H, Me, —CF₃, or it forms a ring together with R₃, a 5- or 6-membered aromatic or non-aromatic ring, all carbon or with O within the ring; R₃ is selected from H, Me, or it forms a ring together with R₂, a 5- or 6-membered aromatic or non-aromatic ring, all carbon or with O within the ring; R₅ is selected from —H and -Me; R₆ and R₆′ are —H; n is 1; R₉ and R₉′ are —H; R₁₀ is —H; R₁₁ is selected from —H or -Me.

In one embodiment, the compound of Formula (2-VII) is further represented by the following:

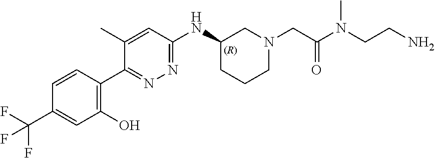

The present disclosure also provides a pharmaceutical composition comprising a therapeutically effective amount of a compound and a pharmaceutically acceptable excipient as described herein.

The present disclosure further provides a method of preventing, treating, or ameliorating one or more diseases in a subject, comprising administering a compound or a pharmaceutically acceptable excipient as described herein, to a subject in need thereof.

In several embodiments, the disease is characterized by a disease progression that comprises the activity of IL-1b, IL-18, or both. NLRP3 inflammasome activation has been linked to various inflammasome-related diseases/disorders, immune diseases, inflammatory diseases, auto-immune diseases and auto-inflammatory diseases. Various eye diseases linked to the activation of the NLRP3 inflammasome, which is a part of the immune system involved in responding to cellular stress and damage, may be treated according to the disclosure. Without being limited to a theory of operation, one possible rationale behind targeting NLRP3 inflammasome activation in these conditions would be due to the role of inflammasome activation in inflammatory and immune responses, which can lead to or exacerbate eye diseases. Accordingly, one aspect of the disclosure is directed to treating, preventing or ameliorating eye diseases.

As a non-limiting example, age-related macular degeneration (AMD), both wet and dry types, involves the deterioration of the central part of the retina, leading to vision loss. NLRP3 inflammasome activation may contribute to the inflammation and neovascularization seen in AMD. Similarly, diabetic macular edema (DME) and diabetic retinopathy (DR) are complications of diabetes where high blood sugar levels damage the retinal blood vessels, and inflammation plays a significant role in their progression.

Diseases like glaucoma, characterized by increased pressure in the eye leading to optic nerve damage, and retinopathy, involving damage to the retinal blood vessels, are also associated with inflammatory responses mediated by the NLRP3 inflammasome. Dry eye disease (DED) and bacterial keratitis, an infection of the cornea, involve inflammation where NLRP3 might play a role in the response to infection and cellular stress.

Inflammatory and autoimmune diseases like Behcet's syndrome, systemic lupus erythematosus (SLE), and rheumatoid arthritis can have ocular manifestations, including uveitis (inflammation of the middle layer of the eye) and retinal vasculitis (inflammation of the retinal vessels). These conditions are often marked by an overactive immune response, where controlling NLRP3 inflammasome activation may manage inflammation and prevent tissue damage. Targeting the NLRP3 inflammasome for patients experiencing, or at risk of experiencing, the various diseases mentioned above may reduce inflammation, slow disease progression, preserve vision, and improve quality of life by addressing one of the underlying mechanisms contributing to these eye conditions.

Consistent with the disclosure, compounds of the disclosure may be used for treating, preventing or ameliorating a disease affecting the eye. In several embodiments, the disease is selected from the group consisting of, for example, age-related macular degeneration (wet and dry) (AMD), atrophic macular degeneration, bacterial keratitis, Behcet's syndrome, choroidal neovascularization, chronic eye disease, diabetic macular edema (DME), diabetic retinopathy (DR), dry eye disease (DED, glaucoma (acute and non-acute), geographic atrophy (GA), and retinopathy.

In several embodiments, the disease is selected from the group consisting of, for example, inflammatory eye disease, ocular inflammation associated with cryopyrin-associated periodic syndrome (CAPS), ocular manifestations of rheumatoid arthritis and systemic lupus erythematosus (SLE), retinitis, retinal vasculitis, retinal vein occlusion (RVO), progressive optic neuropathy, and uveitis (anterior/intermediate/posterior, pan-uveitis).

In some embodiments, the disease is age-related macular degeneration (AMD). In some embodiments, the disease is atrophic macular degeneration. In some embodiments, the disease is bacterial keratitis. In some embodiments, the disease is Behcet's syndrome. In some embodiments, the disease is choroidal neovascularization. In some embodiments, the disease is chronic eye disease. In some embodiments, the disease is diabetic macular edema (DME). In some embodiments, the disease is diabetic retinopathy (DR). In some embodiments, the disease is dry eye disease (DED). In some embodiments, the disease is glaucoma, both acute and non-acute. In some embodiments, the disease is geographic atrophy (GA). In some embodiments, the disease is retinopathy. In some embodiments, the disease is inflammatory eye disease. In some embodiments, the disease is associated with ocular inflammation from cryopyrin-associated periodic syndrome (CAPS). In some embodiments, the disease involves ocular manifestations of rheumatoid arthritis and systemic lupus erythematosus (SLE). In some embodiments, the disease is retinitis. In some embodiments, the disease is retinal vasculitis. In some embodiments, the disease is retinal vein occlusion (RVO). In some embodiments, the disease is progressive optic neuropathy. In some embodiments, the disease is uveitis, including anterior, intermediate, posterior, or pan-uveitis.

In some embodiments, due to activity towards the NLRP3 inflammasome, the compounds of the disclosure are expected to have significant biological activity against a range of systemic diseases. For instance, in diseases like gout and atherosclerosis, where inflammation is a primary factor, compounds of the disclosure may reduce symptoms and progression by modulating the inflammatory response. In several embodiments, the disease is selected from the group consisting of atherosclerosis, gout, acute gouty arthritis, rheumatoid arthritis, nonalcoholic steatohepatitis, inflammatory bowel disease, Parkinson's disease, Alzheimer's disease, multiple sclerosis, (acute) glaucoma, wet age related macula degeneration, dry age related macula degeneration, diabetic retinopathy, Behcet's syndrome, dry eye (disease), and bacterial, viral, fungal, and parasitic infections.

### BRIEF DESCRIPTION OF THE DRAWINGS

FIG. 1 shows IL-1ß release inhibition in U937 cells.

FIG. 2 shows IL-1ß release inhibition in THP-1 cells.

FIG. 3 shows Caspase-1 inhibition in THP-1 cells.

FIG. 4 Shows viability assay in THP-1 cells.

FIG. 5 shows ASC-GFP speck inhibition in THP-1 cells.

FIG. 6 shows the effects of test compounds on TNFα in U937 cells.

FIG. 7 shows the toxicity assessment in human retinal pigment epithelial cells.

### DETAILED DESCRIPTION

The compounds disclosed herein are potent inhibitors of NLRP3 inflammasome. Such NLRP3 inhibitory compounds may be useful in the treatment or prevention of inflammatory disorders, and diseases with underlying pathology of inflammation, associated with NLRP3 inflammasome. Lack of high-resolution crystal structures of ligand bound NLRP3 protein complex makes it a challenge to apply structure-based design approaches in the discovery of NLRP3 inhibitors. A pharmacophore/ligand-based approach was used to identify a structural region where incorporation of a polar amine or amide linked to a terminal hydroxyl group, a terminal primary amino group, or a secondary amino group is tolerated for activity while allowing us to modulate physicochemical properties of the parent compounds of Formula (1-I), (1-II), (1-III), (1-IV), (1-V), (1-VI), (1-VII), (1-VIII), (1-IX), (2-1), (2-II), (2-III), (2-IV), (2-V), (2-VI), and (2-VII).

Surprisingly, incorporating an extended linker containing an amine or amide with a terminal primary, secondary, and tertiary hydroxyl group, or a terminal primary or secondary amino group at a strategic position of the molecular structure, in specific cases, is not only tolerated but also delivered robust NLRP3 inhibitory potency or robust inhibition of IL-1ß secretion, while also facilitating a solubility increase and a lipophilicity reduction for an enhanced safety profile.

Said surprising terminal primary, secondary, and tertiary hydroxyl bearing compounds have the general structures shown by 1A, 1B, 1C, 1D, 1E, 1F and 1G, where Y′ is a partial known pharmacophore or an analog thereof for NLRP3 inhibitory activity or IL-1ß secretion inhibitory activity, such as an appropriately substituted (R)-2-(6-(piperidin-3-ylamino)pyridazin-3-yl)phenol, an appropriately substituted (R)-5-(6-(piperidin-3-ylamino)pyridazin-3-yl)benzo[b]thiophen-4-ol, or an appropriately substituted (R)-2-(8-(piperidin-3-yl)-5,6,7,8-tetrahydropyrido[2,3-c]pyridazin-3-yl)phenol, wherein

X is a cycloalkyl, substituted cycloalkyl, heterocycloalkyl, or substituted heterocycloalkyl ring (including substituted and non-substituted spirocyclic and bicyclic rings), X₂ is a heterocycloalkyl or substituted heterocycloalkyl ring (including spirocyclic rings and substituted spirocyclic rings), X₃ is a heterocycloalkyl or substituted heterocycloalkyl ring (including substituted and non-substituted spirocyclic rings), X₄ is a cycloalkyl ring or a substituted cycloalkyl ring, X₅ is a heterocycloalkyl ring or a substituted heterocycloalkyl ring (including spirocyclic rings or substituted spirocyclic rings), X₆ is a heterocycloalkyl ring or a substituted heterocycloalkyl ring (including spirocyclic rings or substituted spirocyclic rings), X₇ is a heterocycloalkyl ring or a substituted heterocycloalkyl ring (including spirocyclic rings or substituted spirocyclic rings), X₈ is a heterocycloalkyl ring or a substituted heterocycloalkyl ring (including spirocyclic rings or substituted spirocyclic rings), R₄ is optional and if present is —F or -Me, -Et, iPr or cPr, and W is NH, N-Me, N-Et or N-iPr, or W is a bond (when W is a bond, ring

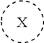

is attached directly to the carbonyl via an a nitrogen atom embedded in the heterocyclic ring

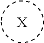

to form an amide); Y is a bond (when Y is a bond, the —OH is attached directly to the ring) or Y is C1-C3 alkyl, optionally substituted with 1, 2, or 3 C1-C3 alkyl, or —CF₃.

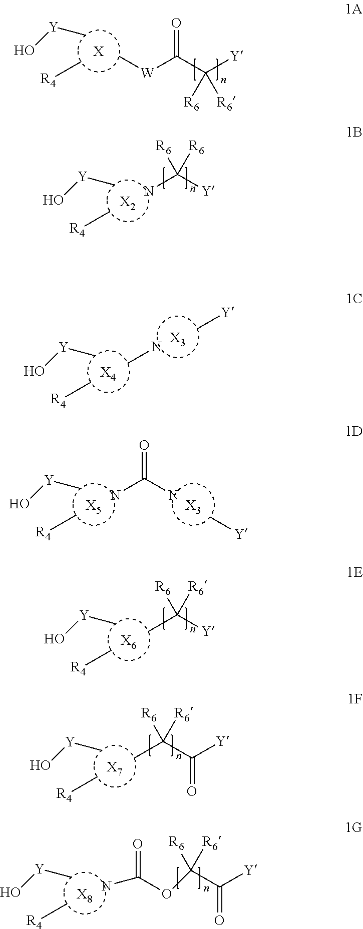

Said surprising terminal primary or secondary amine bearing compounds have the general structures shown by 2A, 2B, 2C, 2D, 2E, 2F, and 2G.

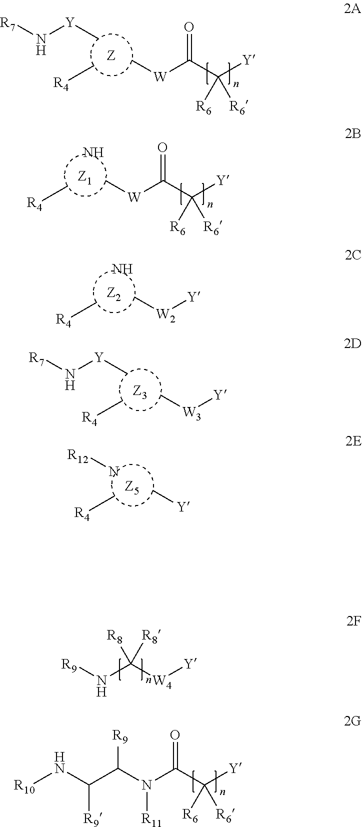

wherein Y′ is a partial known pharmacophore or an analog thereof for NLRP3 inhibitory activity or IL-1ß secretion inhibitory activity, such as an appropriately substituted (R)-2-(6-(piperidin-3-ylamino)pyridazin-3-yl)phenol, an appropriately substituted (R)-5-(6-(piperidin-3-ylamino)pyridazin-3-yl)benzo[b]thiophen-4-ol, or an appropriately substituted (R)-2-(8-(piperidin-3-yl)-5,6,7,8-tetrahydropyrido[2,3-c]pyridazin-3-yl)phenol, wherein wherein ring

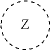

is a 4-11 membered ring, selected from cycloalkyl, substituted cycloalkyl, heterocycloalkyl, or substituted heterocycloalkyl, including spirocyclic and bicyclic cycloalkyl, substituted spirocyclic and bicyclic, spirocyclic and bicyclic heterocycloalkyl, and substituted spirocyclic and bicyclic heterocycloalkyl, and where W is selected from NH, N-Me, N-Et, and N-iPr, or is a bond; when W is a bond, the carbonyl group (C═O) is bonded to a nitrogen atom within ring

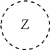

so as together they form an amide bond; R₄ is selected from —H, —F, C1-C3 alkyl, and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 1, 2, 3, and 4; R₇ is selected from —H, C1-C6 alkyl, cycloalkyl, heterocycloalkyl,

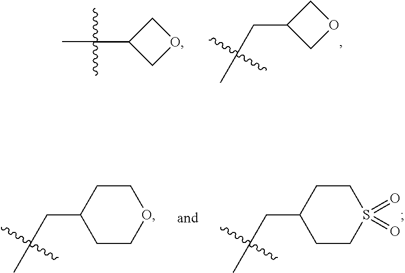 [SMILES: *C1COC1 ; *CC1COC1 ; *CC1CCOCC1 ; *CC1CCS(=O)(=O)CC1]

ring

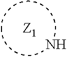

is a 4-9 membered nitrogen-containing ring, including heterocycloalkyl ring and substituted heterocycloalkyl ring, including spirocyclic and bicyclic nitrogen-containing heterocycloalkyl and substituted spirocyclic and bicyclic heterocycloalkyl, and where W is selected from NH, N-Me, N-Et and N-iPr, or is a bond; when W is a bond, the carbonyl group (C═O) is bonded to a nitrogen atom within ring

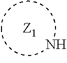

so as together they form an amide bond; ring

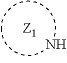

is a 4-9 membered nitrogen-containing heterocycloalkyl ring, including spirocyclic and bicyclic nitrogen-containing heterocycloalkyl; Y is a bond (when Y is a bond, the —OH is attached directly to the ring) or Y is C1-C3 alkyl, optionally substituted with 1, 2, or 3 C1-C3 alkyl, or —CF₃; ring

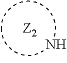

is a 4-11 membered ring, selected from heterocycloalkyl or substituted heterocycloalkyl, including spirocyclic and bicyclic heterocycloalkyl, and substituted spirocyclic and bicyclic heterocycloalkyl; W₂ is selected from optionally-substituted —CH₂—, —CH₂CH₂—, —CH₂CH₂CH₂—, or is a bond; ring

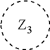

is a 4-9 membered ring, selected from cycloalkyl, substituted cycloalkyl, heterocycloalkyl or substituted heterocycloalkyl, including spirocyclic and bicyclic cycloalkyl, substituted spirocyclic and bicyclic, spirocyclic and bicyclic heterocycloalkyl, and substituted spirocyclic and bicyclic heterocycloalkyl; W₃ is selected from optionally-substituted —CH₂—, —CH₂CH₂—, —CH₂CH₂CH₂—, or is a bond; R₇ is selected from —H, C1-C6 alkyl or cycloalkyl,

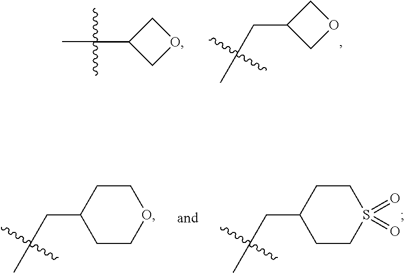 [SMILES: *C1COC1 ; *CC1COC1 ; *CC1CCOCC1 ; *CC1CCS(=O)(=O)CC1]

ring

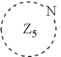

is azetidine, pyrrolidine, piperidine or azepane; R₁₂ is selected from:

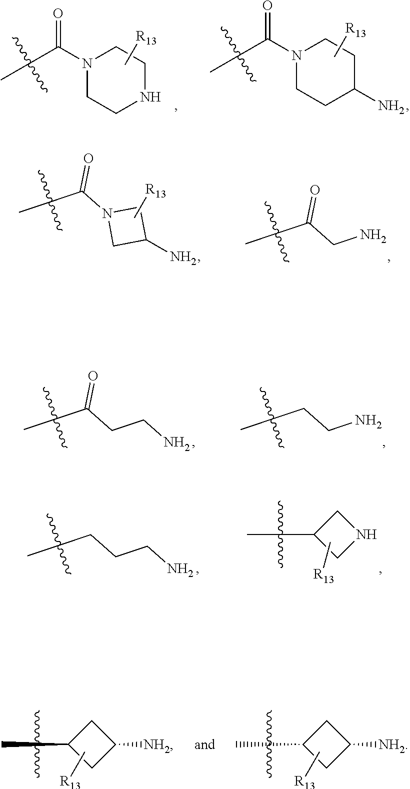

Rings may have 1, 2, or 3 R₁₃ groups, with each R₁₃ independently selected from —H, C1-C3 alkyl, and —CF₃. Two R₁₃ groups may connect to form a cyclopropyl or cyclobutyl ring; W₄ is selected from —CH₂—, —CH₂CH₂—,

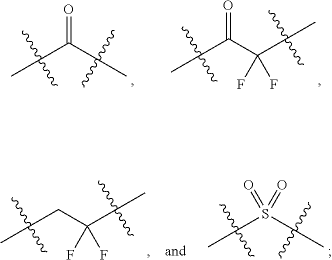

R₈ and R₈′ are independently selected from —H, —F, C1-C5 alkyl (including cycloalkyl), and —CF₃; R₉ is independently selected from —H, C1-C6 alkyl, or cycloalkyl,

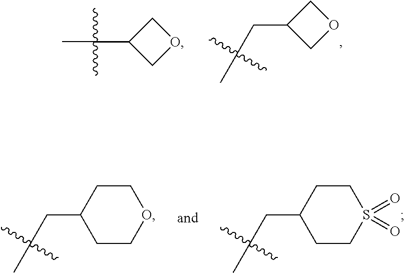 [SMILES: *C1COC1 ; *CC1COC1 ; *CC1CCOCC1 ; *CC1CCS(=O)(=O)CC1]

or R₉ and R₉, together with the atoms that they are bonded to form a (substituted) C3-C6 cycloalkyl or (substituted) C3-C6 heterocycloalkyl ring; R₁₀ is selected from —H, C1-C7 alkyl (including cycloalkyl and methylcycloalkyl),

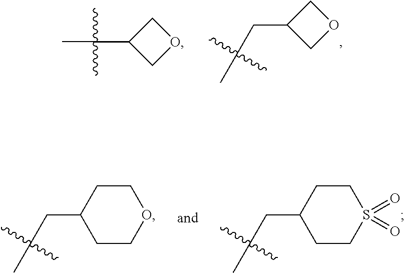 [SMILES: *C1COC1 ; *CC1COC1 ; *CC1CCOCC1 ; *CC1CCS(=O)(=O)CC1]

R₁₁ is selected from H, C1-C7 alkyl (including cycloalkyl and methylcycloalkyl).

As such, the compounds disclosed not only are potent inhibitors of NLRP3 inflammasome or IL-1ß secretion, but also have structural components that can contribute to the enhanced safety profile of a drug or improve therapeutic utility via better physicochemical properties.

### Definitions

For the purpose of the present invention the following terminology will be used in accordance with the definitions set forth below.

“A” and “an” are used herein to refer to one or more than one (e.g., to at least one) of the grammatical object of the article. By way of example, “an element” means one element or more than one element.

“And/or” is used herein to mean either “and” or “or” unless indicated otherwise.

“About” as used herein means variation one might see in measurements taken among different instruments, samples, and sample preparations.

“Administer”, “administering”, or “administration” as used herein refers to either directly administering a disclosed compound or pharmaceutically acceptable salt of the disclosed compound or a composition thereof to a subject or administering a prodrug derivative or analog of the compound or pharmaceutically acceptable salt of the compound or composition thereof to the subject, which can form an equivalent amount of active compound within the subject's body.

“Alkyl” refers to a straight or branched, saturated, aliphatic radical. The number of carbon atoms present in the alkyl group may be specified by indicating the number of carbon atoms in the group (e.g., C3 alkyl contains three carbon atoms). The size range of an alkyl group can be specified by indicating a range of the numbers of carbon atoms (e.g., C1-C3 alkyl or (C1-C3)alkyl or C1-3 alkyl for a one to three carbon atom containing alkyl group). For example, C1-C6 alkyl includes, but is not limited to, methyl (also referred to herein as “-Me”), ethyl, propyl, isopropyl, n-butyl, isobutyl, sec-butyl, tert-butyl, pentyl, isopentyl, hexyl, etc. Non-limiting examples of alkyl groups include methyl, ethyl, propyl, butyl, pentyl, 1-methylbutyl (i.e., 2-pentyl), 1-ethylpropyl (i.e., 3-pentyl), 3-methylpentyl, and the like. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together, and it is understood that “alkyl” includes alkylene when two functionalities are appended through a straight or branched, saturated, aliphatic diradical. Consistent with other groups and substituents, this group may be optionally substituted with other groups.

“Heteroalkyl” refers to an alkyl group having from 1 to 3 heteroatoms such as N, O, and S. The heteroatoms can also be oxidized, such as, but not limited to, —S(O)— and —S(O)₂—, or —N—OH and —N⁺—O⁻. For example, heteroalkyl can include ethers, thioethers, alkyl-amines and alkyl-thiols. The heteroalkyl group is typically monovalent, but can be divalent or multivalent, such as when the heteroalkyl group links two or more moieties together, and it is understood that “heteroalkyl” includes heteroalkylene when two functionalities are appended. Consistent with other groups and substituents, this group may be optionally substituted with other groups.

“Cycloalkyl” or “carbocyclyl” refers to a cyclic hydrocarbon group that contains from 3 to 12, from 3 to 10, from 3 to 8, or from 3 to 7 endocyclic carbon atoms. Cycloalkyl groups include fused, bridged and spiro ring structures. Where cycloalkyl groups may have a range of sizes, that size range may be specified by indicating the number of carbon atoms present in the cycloalkyl group (e.g., 3 to 10 membered cycloalkyl ring, C3-C10 or (C₃-C₁₀) cycloalkyl or C₃₋₁₀ cycloalkyl for a three to ten carbon atom containing cycloalkyl group). Examples include cyclopropanyl, cyclobutanyl, cyclopentanyl, methylcyclopentanyl, cyclohexanyl, methylcyclohexanyl, cycloheptanyl, and dimethylcyclohexanyl. Cycloalkyls may be optionally substituted. Cycloalkyls may include spirocycles, such as the nonlimiting example of spiro[4.5]decane. Consistent with other groups and substituents, this group may be optionally substituted with other groups.

“Heterocyclic” refers to a ring system having from 3 ring members to about 20 ring members and from 1 to about 5 heteroatoms such as N, O and S. The heteroatoms can also be oxidized, such as, but not limited to, —S(O)— and —S(O)₂—, or —N—OH and —N⁺—O⁻. For example, heterocycle includes, but is not limited to, tetrahydrofuranyl, tetrahydropyranyl, oxepanyl, tetrahydrothiophenyl, morpholino, pyrrolidinyl, imidazolidinyl, imidazolinyl, pyrazolidinyl, pyrazolinyl, piperazinyl, piperidinyl, azepanyl, indolinyl, quinuclidinyl and 1,4-dioxa-8-aza-spiro[4.5]dec-8-yl. Heterocyclic group includes heterocycloalkyl and heteroaryl. Consistent with other groups and substituents, this group may be optionally substituted with other groups.

“Heterocycloalkyl” refers to a group of cyclic compounds characterized by having at least one non-carbon atom, such as nitrogen, oxygen, or sulfur, in the ring structure. The heterocycloalkyl groups can appear in various forms, including monocyclic, bicyclic, and spirocyclic configurations. Monocyclic heterocycloalkyls have a single ring containing one or more heteroatoms. Examples include pyrrolidine, which has a five-membered ring with one nitrogen atom; tetrahydrofuran (THF), a five-membered ring with oxygen; and thiolane, featuring a sulfur atom in its five-membered ring. Bicyclic heterocycloalkyls have two connected rings which can both be heterocyclic or a combination of heterocyclic and carbocyclic (all-carbon ring). Spirocyclic heterocycloalkyls are compounds where two or more rings are connected via a single shared atom, typically a quaternary carbon, known as the spiroatom. Spiro[4.5]decane-7,9′-dione is an example where a five-membered lactone ring and a six-membered ketone ring share a single carbon atom. Spiropiperidine connects a piperidine ring to another cyclic structure through a shared spiroatom. Consistent with other groups and substituents, this group may be optionally substituted with other groups.

“Alkoxy” refers to a straight or branched chain saturated or unsaturated (fully or partially) hydrocarbon containing 1-12 carbon atoms containing a terminal “O” in the chain, e.g., —O(alkyl). Examples of alkoxy groups include without limitation, methoxy, ethoxy, propoxy, butoxy, t-butoxy, or pentoxy groups. In an embodiment, “alkoxy” is fully saturated. Consistent with other groups and substituents, this group may be optionally substituted with other groups.

“Alkoxyalkoxy” refers to an alkoxy group as defined herein which is substituted with an alkoxy group e.g., —O(alkyl)-O-(alkyl). Examples of alkoxyalkoxy groups include, without limitation, methoxymethoxy, ethoxyethoxy, propoxymethoxy, or ethoxymethoxy. Consistent with other groups and substituents, this group may be optionally substituted with other groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched. It is understood that an alkenyl group, as defined herein, can be a pure geometric isomer, e.g., and a pure (E)- or a pure (Z)-isomer, or a mixture of geometric isomers, e.g., (E)/(Z)-isomers, also known as trans/cis-isomers, of any ratio. Consistent with other groups and substituents, this group may be optionally substituted with other groups.

“Alkynyl” refers to a straight or branched chain unsaturated hydrocarbon containing 2-12 carbon atoms. The “alkynyl” group contains at least one triple bond in the chain. Examples of alkynyl groups include ethynyl, propynyl, n-butynyl, iso-butynyl, pentynyl, or hexynyl. An alkynyl group can be unsubstituted or substituted. Consistent with other groups and substituents, this group may be optionally substituted with other groups.

“Aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di-, tri- or higher substituted by one, two, three or more radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl. Consistent with other groups and substituents, this group may be optionally substituted with other groups.

“Heteroatom” refers to an atom that is not a carbon atom and is part of the contiguous cyclic structure of a cyclic compound or part of the continuous structure of a linear or branched compound. Consistent with other groups and substituents, this group may be optionally substituted with other groups.

“Heteroaryl” refers to a monocyclic or fused bicyclic or tricyclic aromatic ring assembly containing 5 to 16 ring atoms, where from 1 to 4 of the ring atoms are each a heteroatom independently selected from N, O and S. Non-limiting examples of heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl. Consistent with other groups and substituents, this group may be optionally substituted with other groups.

Substituents for the cycloalkyl, heterocycloalkyl, aryl and heteroaryl groups are varied and are selected from: halogen, —OR′, —OC(O)R′, —NR′R″, —NR′OH, —NR′OR″, —N⁺R′R″—O⁻, —SR′, —R′, —CN, —NC, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)—NR′, —N⁺(—O⁻)(═CR′R″), —S(O)R′, —S(O)₂R′, —S(O)₂NR′R″, —N₃, —CH(Ph)₂, perfluoro (C1-C4)alkoxy, and perfluoro (C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl. Consistent with the disclosure, nonlimiting example of substituents may be substituted on the groups of the description.

“Carrier”, as used herein, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

“Compound” as used herein is intended to encompass not only the specified molecular entity but also its pharmaceutically acceptable, pharmacologically active derivatives, including, but not limited to, salts, prodrug, metabolites, hydrates, solvates and the like.

“Composition” as used herein means a mixture of substances suitable for administering to a test subject or individual that includes a pharmaceutical agent. In some embodiments, said test subject is a mammal. In some embodiments, said test subject is a human.

“Cyano” as used herein means a substituent having a carbon atom joined to a nitrogen atom by a triple bond, e.g., —C═N.

“Disorder” is used herein to mean, and is used interchangeably with, the terms disease, condition, or illness, unless otherwise indicated.

“Halogen” or “halo” refers to fluorine, chlorine, bromine, or iodine.

“Haloalkyl” as used herein refers to an alkyl group, as defined herein, which is substituted by one or more halogen. Examples of haloalkyl groups include, but are not limited to, trifluoromethyl, difluoromethyl, pentafluoroethyl, trichloromethyl, etc. In an embodiment, “haloalkyl” is fully saturated. In embodiments where the haloalkyl group is chiral, for example, but not construed to be limiting, —CH(X)—CH₃, —CH₂—CH(X)—CH₃, or the like, and where X is a halogen atom, all possible stereoisomers are included. Consistent with other groups and substituents, this group may be optionally substituted with other groups.

“Haloalkoxy” as used herein refers to an alkoxy group, as defined herein, which is substituted one or more halogen. Examples of haloalkyl groups include, but are not limited to, trifluoromethoxy, difluoromethoxy, pentafluoroethoxy, trichloromethoxy, etc. In an embodiment, “haloalkoxy” is fully saturated. Consistent with other groups and substituents, this group may be optionally substituted with other groups.

“Hydroxyalkyl” means an alkyl group as defined above, where the alkyl group is substituted with one or more —OH groups. Examples of hydroxyalkyl groups include HOCH₂—, HO—CH₂—CH₂— and CH₃—CH(OH)—. In an embodiment, “hydroxyalkyl” is fully saturated. In embodiments where the hydroxyalkyl group is chiral, for example, but not construed to be limiting, —CH(OH)—CH₃, —CH₂—CH(OH)—CH₃, or the like, all possible stereoisomers are included. Consistent with other groups and substituents, this group may be optionally substituted with other groups.

“Isomer” refers to certain compound of the present invention which possess asymmetrically substituted carbon atoms (stereogenic centers) or double bonds; the racemate, diastereomer, atropisomer, geometric isomer and individual isomer (e.g., separate enantiomers). All of these are encompassed by the term “isomer” within the scope of the present disclosure.

“Optionally substituted” is understood to mean that a given chemical moiety (e.g., an alkyl, cycloalkyl, heterocycloalkyl, heteroalkyl, aryl or heteroaryl) can (but is not required to) be bonded to other substituents (e.g., heteroatoms). For instance, an alkyl group that is optionally substituted can be a fully saturated alkyl chain (e.g., a pure hydrocarbon). Alternatively, the same optionally substituted alkyl, cycloalkyl, heterocycloalkyl, heteroalkyl, aryl or heteroaryl group can have substituents different from hydrogen. For instance, it can, at any point along the chain, be bound to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkenyl, (C₁-C₆)alkynyl, (C₁-C₆) hydroxyalkyl, (C₁-C₆)alkoxy, (C₁-C₆) haloalkyl, (C₁-C₆) haloalkoxy, (C₃-C₇) cycloalkyl, aryl, heterocycloalkyl, heteroaryl, —O—(C₂-C₆)alkenyl, —O—(C₂-C₆)alkynyl, (C₂-C₆)alkenyl, (C₂-C₆)alkynyl, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)₂NH(C₁-C₆)alkyl, and S(O)₂N((C₁-C₆)alkyl)₂. “Optionally substituted” as used herein also refers to substituted or unsubstituted whose meaning is described below.

“Substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

“Unsubstituted” means that the specified group bears no substituents.

“Oxo” as used herein refers to an “═O” group.

“Patient” or “subject in need thereof” refers to a living organism suffering from or prone to a condition that can be prevented or treated by administration of a pharmaceutical composition as provided herein. Non-limiting examples include humans, other mammals and other non-mammalian animals.

“Pharmaceutically acceptable composition” or “pharmaceutical composition” refers to a composition comprising a compound of the invention and a pharmaceutically acceptable excipient or pharmaceutically acceptable excipients.

“Pharmaceutically acceptable excipient” and “pharmaceutically acceptable carrier” refer to an excipient that can be included in the compositions of the invention and that causes no significant adverse toxicological effect on the patient. Non-limiting examples of pharmaceutically acceptable excipients include water, NaCl, normal saline solutions, lactated Ringer's, normal sucrose, normal glucose and the like.

“Prodrug” is a medication or compound that, after administration is metabolized (i.e., converted within the body of a subject including but not limited to mammals or humas) into a pharmacologically active drug. Instead of administering a drug directly, a corresponding prodrug may be used, for example but not limiting to, to improve how the drug is absorbed, distributed, metabolized, and excreted or how selectively the drug interacts with cells or processes that are not its intended target. The term “prodrug” encompasses the subset of polymeric prodrugs, where the active drug is attached (typically covalently) to a polymer chain, creating a macromolecular carrier system. In some examples, the polymer may improve the solubility and stability of the drug, provide a controlled and/or sustained release mechanism, and can be designed to target the drug to specific tissues, cells, or receptors. The bond between the drug and the polymer is typically cleavable, breaking down under specific physiological conditions to release the active drug at the desired site of action.

“Salt” includes, without limitation, acid addition salts including hydrochlorides, hydrobromides, phosphates, sulphates, hydrogen sulphates, alkylsulphonates, arylsulphonates, acetates, benzoates, citrates, maleates, fumarates, formate, succinates, lactates, and tartrates; salts of alkali metal cations such as Na⁺, K⁺, Li⁺ or alkali earth metal salts such as Mg⁺⁺ or Ca⁺⁺ salts. Pharmaceutically acceptable salts of ionizable pharmaceutical compounds are well known in the art and include, for example, those described in Salts of Therapeutic Agents: Chemical, Physicochemical, and Biological Considerations, Gupta, et. al., Molecules. 2018, 23 (7), 1719, or Pharmaceutical Salts: Properties, Selection, and Use, 2nd Revised Edition, (Stahl, P. H. and Wermuth, C. M., Editors), Wiley-VCH, 2011, Germany both of which are included herein in their entirety.

“Solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates, wherein water is the solvent molecule, are typically referred to as hydrates. Hydrates include compositions containing stoichiometric amounts of water, as well as compositions containing variable amounts of water.

“Spirocycloalkyl” or “spirocyclyl” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom. In an embodiment, “spirocycloalkyl” or “spirocyclyl” is fully saturated. The term “spiroheterocycloalkyl” or “spiroheterocyclyl” is understood to mean a spirocycle wherein at least one of the rings is a heterocycle (e.g., at least one of the rings is furanyl, morpholinyl, or piperadinyl). In an embodiment, “spiroheterocycloalkyl” or “spiroheterocyclyl is fully saturated.

“Therapeutically effective amount” refers to an amount of a biologically active agent or of a pharmaceutical composition useful for treating, ameliorating, or preventing an identified disease or condition, or for exhibiting a detectable therapeutic or inhibitory effect. The effect can be detected by any assay method known in the art.

“Treating” with regard to a subject, refers to improving at least one symptom of the subject's disorder. Treating includes curing, improving, or at least partially ameliorating the disorder.

### Compounds

Provided herein are compounds that are capable of inhibiting NLRP3 family proteins and NLRP3 inflammasome function and inhibition of IL-1ß secretion. Various embodiments of these compounds include compounds having the structures of Formula (1-I), Formula (1-II), Formula (1-III), Formula (1-IV), Formula (1-V), Formula (1-VI), Formula (1-VII), Formula (1-VIII), Formula (1-IX), Formula (2-I), Formula (2-II), Formula (2-III), Formula (2-IV), Formula (2-V), Formula (2-VI), and Formula (2-VII) as described herein or pharmaceutically acceptable salts, solvates, atropisomers, N-oxides, or tautomers thereof.

The structure of compounds of Formula (1-I), Formula (1-II), Formula (1-III), Formula (1-IV), Formula (1-V), Formula (1-VI), Formula (1-VII), Formula (1-VIII), Formula (1-IX), Formula (2-I), Formula (2-II), Formula (2-III), Formula (2-IV), Formula (2-V), Formula (2-VI), and Formula (2-VII) encompass all stereoisomers and racemic mixtures, including the structures described herein.

The present disclosure provides a compound represented by the structure of Formula (1-I):

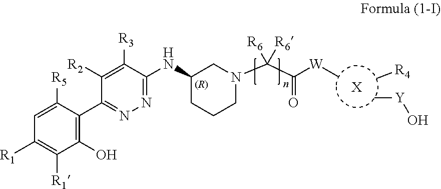

wherein ring

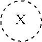

is a 4-11 membered ring, selected from cycloalkyl, substituted cycloalkyl, heterocycloalkyl or substituted heterocycloalkyl, including spirocyclic and bicyclic cycloalkyl, substituted spirocyclic and bicyclic, spirocyclic and bicyclic heterocycloalkyl, and substituted spirocyclic and bicyclic heterocycloalkyl, and where W is selected from —NH, N-Me, N-Et and N-iPr, or is a bond (when W is a bond, ring

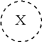

is attached directly to the carbonyl via a nitrogen atom embedded in the ring

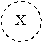

to form an amide), and; R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; Y is a bond (when Y is a bond, the —OH is attached directly to the ring) or Y is C1-C3 alkyl, optionally substituted with 1, 2, or 3 C1-C3 alkyl, or —CF₃; R₄ is selected from —H, —F, C1-C3 alkyl, and —CF₃; R₅ is selected from —H, -Me and —CF₃; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 1, 2, 3, and 4.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-I) is further represented by any one of the following:

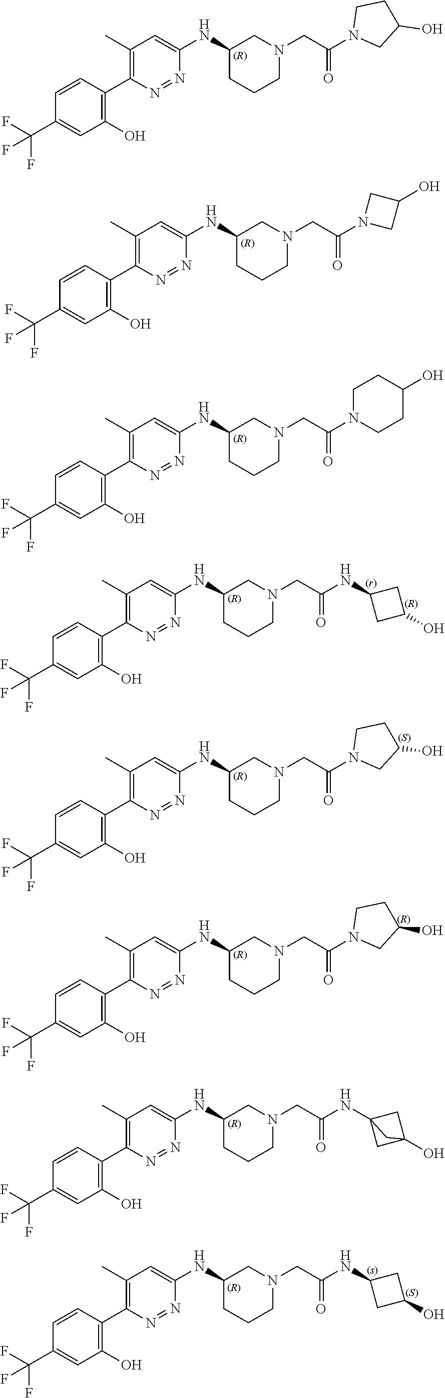

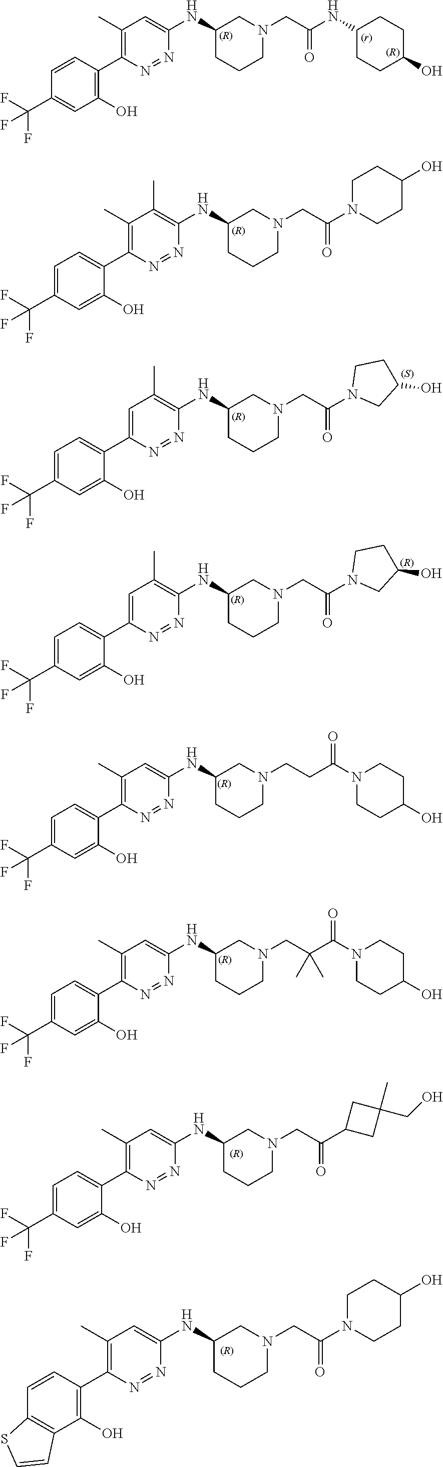

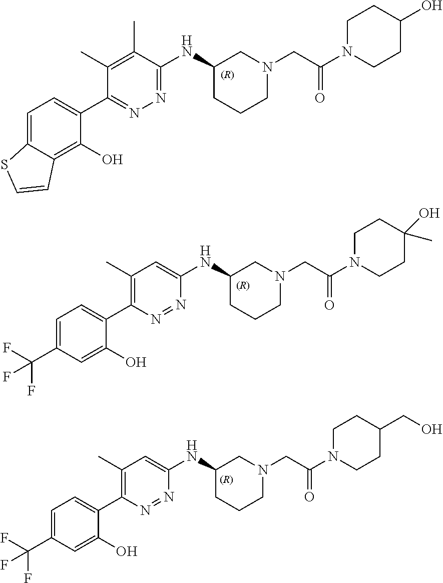

In several embodiments, the compound of Formula (1-I) is further represented by any one of the following:

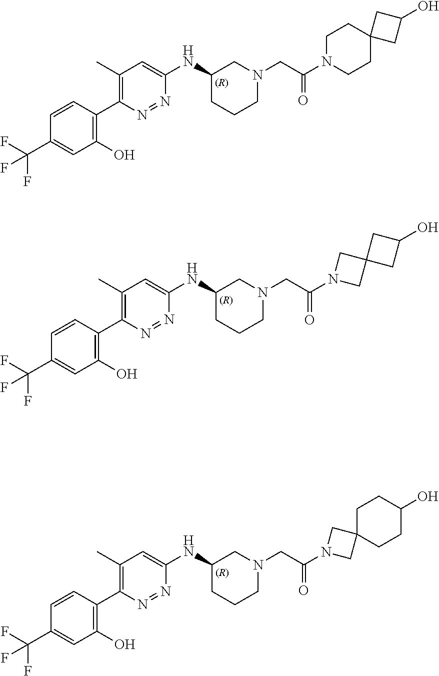

In several embodiments, the compound of Formula (1-1) is further represented by any one of the following:

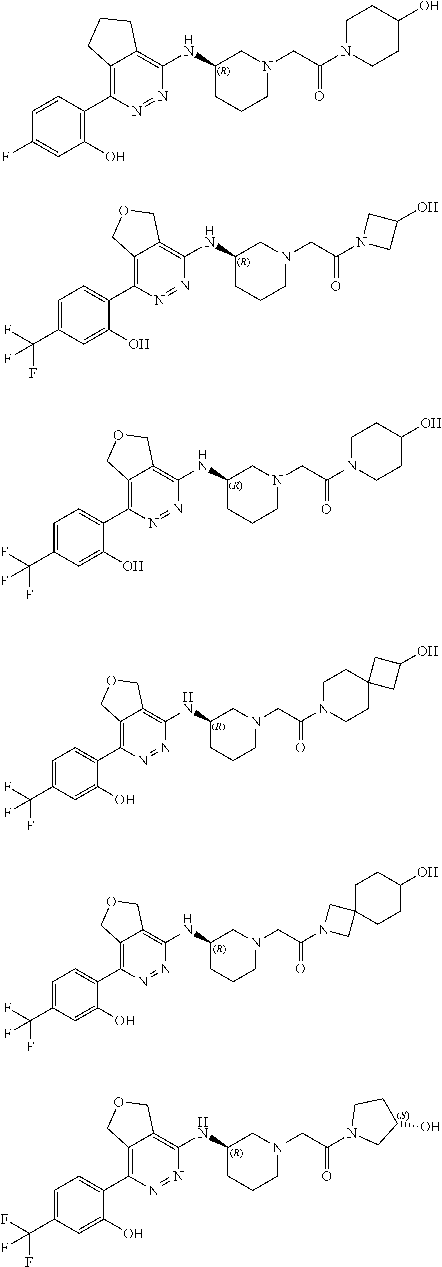

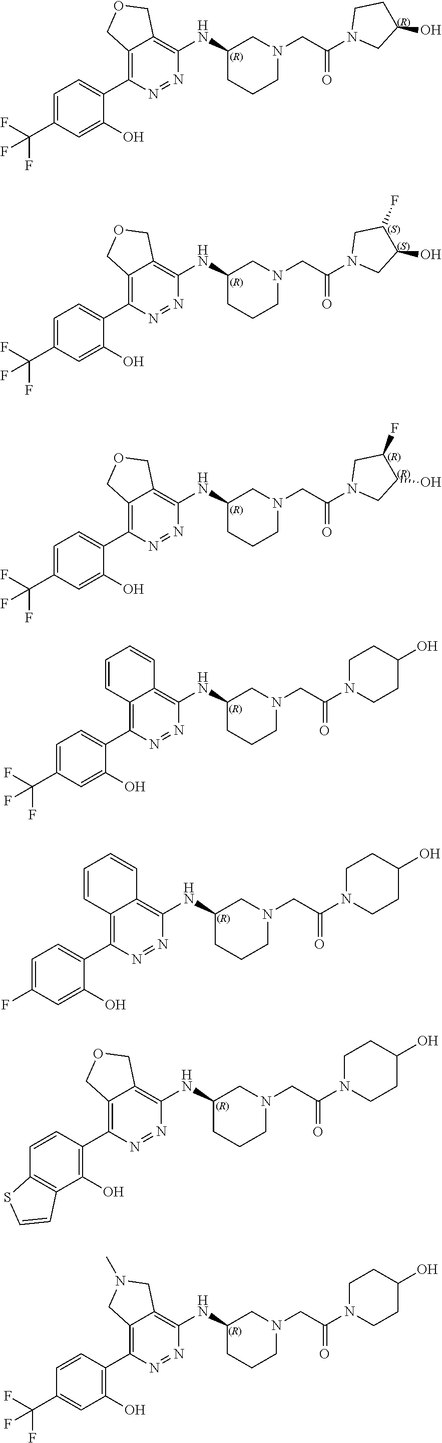

The present disclosure also provides a compound represented by the structure of Formula (1-II):

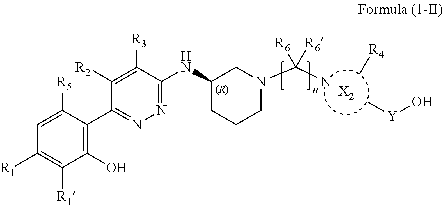

wherein ring

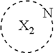

is a 4-11 membered nitrogen-containing heterocycloalkyl ring and substituted heterocycloalkyl ring, including spirocyclic and bicyclic nitrogen-containing heterocycloalkyl and substituted spirocyclic and bicyclic nitrogen-containing heterocycloalkyl rings, and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring with R₃, a 5- or 6-membered aromatic or non-aromatic ring, all carbon or within O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 2, 3, and 4.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-II) is further represented by any one of the following:

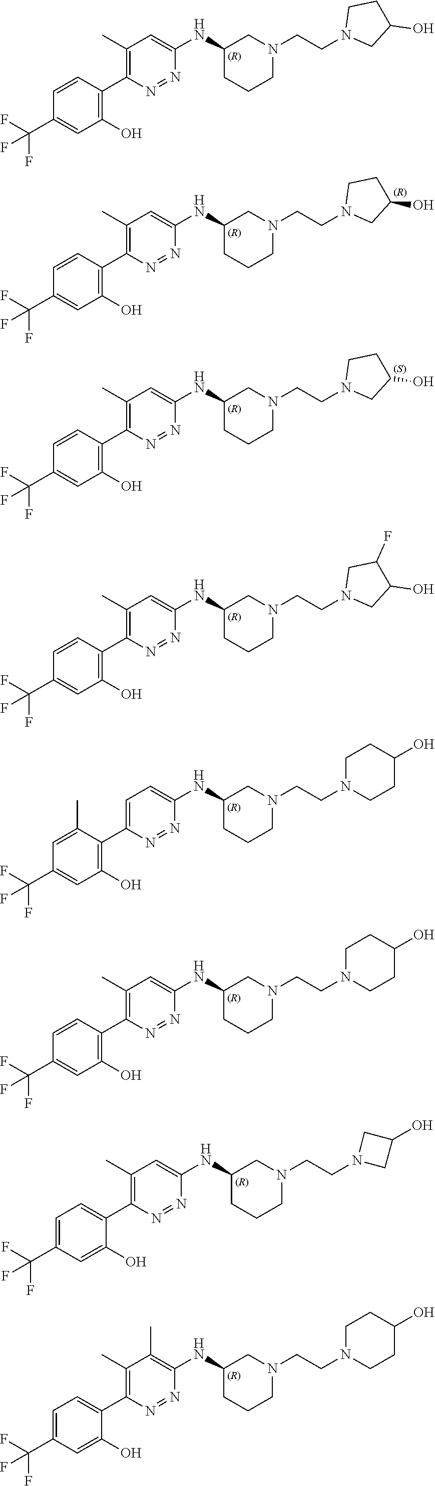

In several embodiments, the compound of Formula (1-II) is further represented by any one of the following:

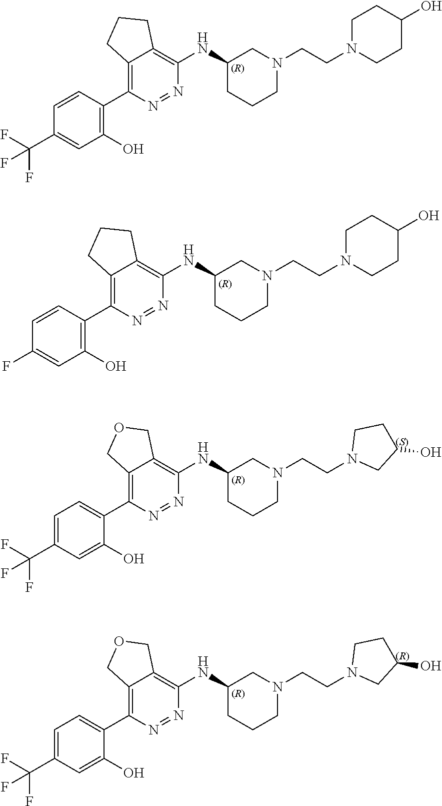

The present disclosure also provides a compound represented by the structure of Formula (1-III):

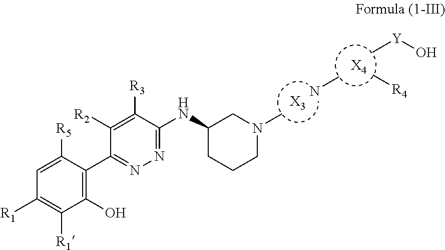

wherein ring

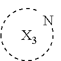

is a 4-7 membered nitrogen-containing heterocycloalkyl ring and substituted heterocycloalkyl ring, including spirocyclic nitrogen-containing heterocycloalkyl and substituted spirocyclic nitrogen-containing heterocycloalkyl rings, wherein ring

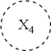

is a 4-7 membered cycloalkyl ring and substituted cycloalkyl ring, including spirocyclic and substituted spirocyclic rings, and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5 or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N in the ring, and each of the C- or N-atoms within the 5- or 6-membered aromatic or non-aromatic ring is optionally substituted with -Me or —OH; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-III) is further represented by any one of the following:

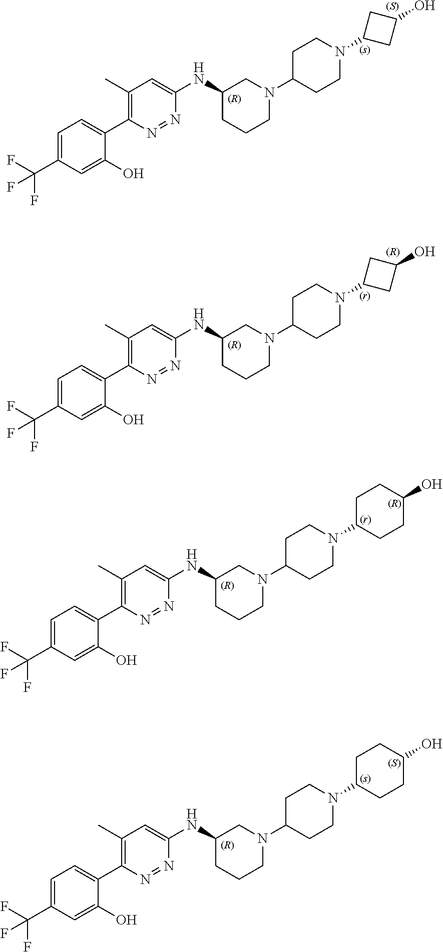

The present disclosure also provides a compound represented by the structure of Formula (1-IV):

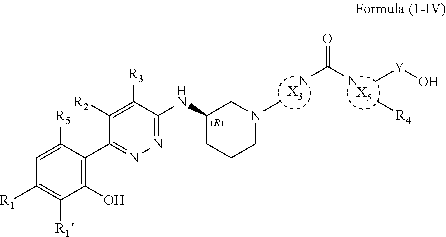

wherein ring

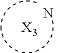

is a 4-7 membered nitrogen-containing heterocycloalkyl ring and substituted heterocycloalkyl ring, including spirocyclic nitrogen-containing heterocycloalkyl and substituted spirocyclic nitrogen-containing heterocycloalkyl rings; wherein ring

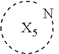

is a 4-7 membered nitrogen-containing heterocycloalkyl ring substituted hetereocycloalkyl ring, including spirocyclic nitrogen-containing heterocycloalkyl and substituted spirocyclic nitrogen-containing heterocycloalkyl rings, and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring optionally substituted with -Me or —OH; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-IV) is further represented by any one of the following:

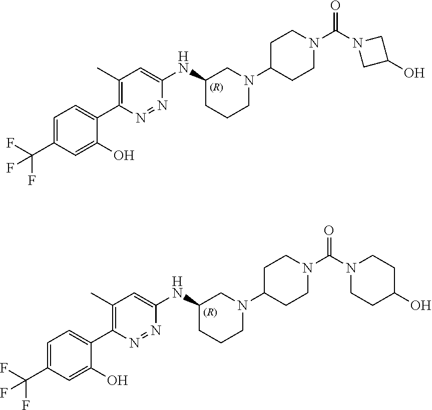

The present disclosure also provides a compound represented by the structure of Formula (1-V):

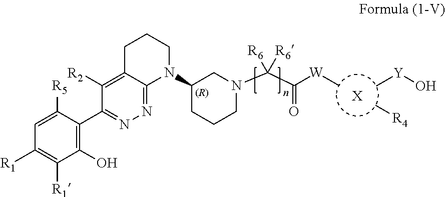

wherein ring

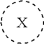

is a 4-11 membered ring, selected from cycloalkyl, substituted cycloalkyl, heterocycloalkyl, or substituted heterocycloalkyl, including spirocyclic and bicyclic cycloalkyl, substituted spirocyclic and bicyclic cycloalkyl rings, spirocyclic and bicyclic heterocycloalkyl, and substituted spirocyclic and bicyclic heterocycloalkyl rings, and where W is selected from NH, N-Me, N-Et and N-iPr, or W is a bond (when W is a bond, ring

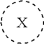

is attached directly to the carbonyl via an nitrogen atom embedded in ring

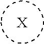

to form an amide), and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃; R₆ and R₆′ are independently selected from —H, —F, C1-C6 alkyl, and —CF₃, or R₆ and R₆′ together with the atom they are bonded to form a C1-C6 cycloalkyl or C1-C6 heterocycloalkyl ring; n is selected from 1, 2, 3, and 4.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-V) is further represented by any one of the following:

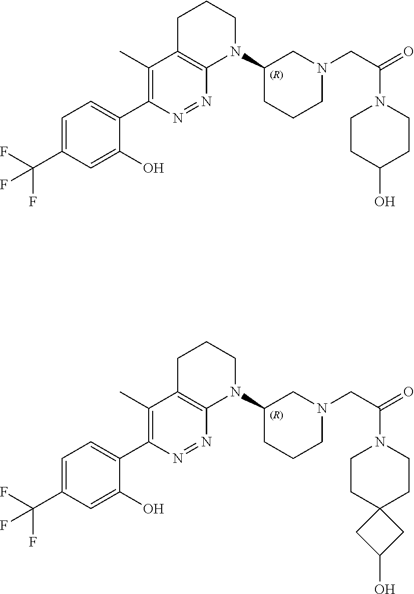

The present disclosure also provides a compound represented by the structure of Formula (1-VI):

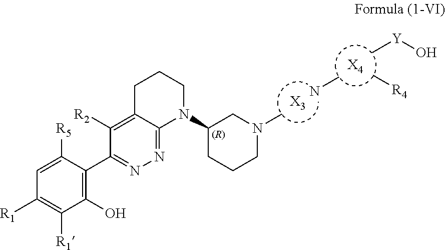

wherein ring

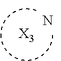

is a 4-7 membered nitrogen-containing heterocycloalkyl ring and substituted heterocycloalkyl ring, including spirocyclic nitrogen-containing heterocycloalkyl and substituted spirocyclic nitrogen-containing heterocycloalkyl rings, wherein ring

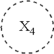

is a 4-7 membered cycloalkyl ring and substituted cycloalkyl ring, including spirocyclic and substituted spirocyclic rings, and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-VI) is further represented by any one of the following:

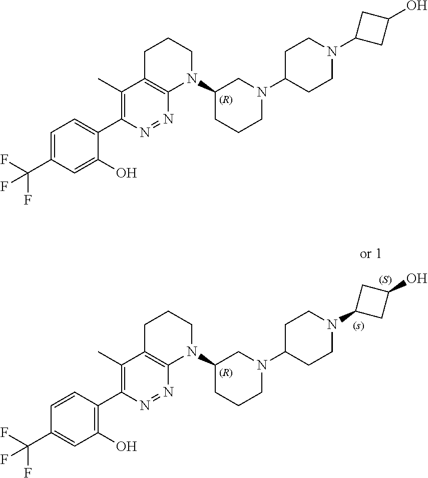

The present disclosure also provides a compound represented by the structure of Formula (1-VII

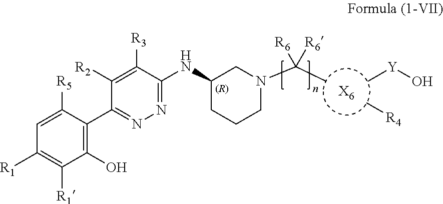

wherein ring

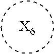

is a 4-11 membered ring, selected from cycloalkyl, substituted cycloalkyl, heterocycloalkyl, or substituted heterocycloalkyl, including spirocyclic and bicyclic cycloalkyl, substituted spirocyclic and bicyclic cycloalkyl rings, spirocyclic and bicyclic heterocycloalkyl, and substituted spirocyclic and bicyclic heterocycloalkyl rings, and R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 0, 1, 2, 3, and 4.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-VII) is further represented by any one of the following:

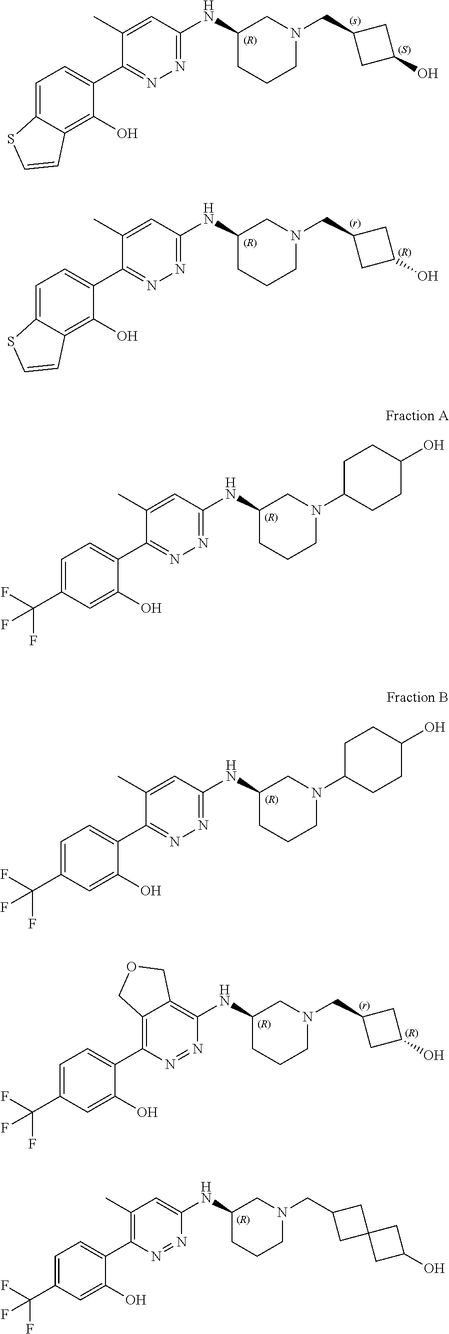

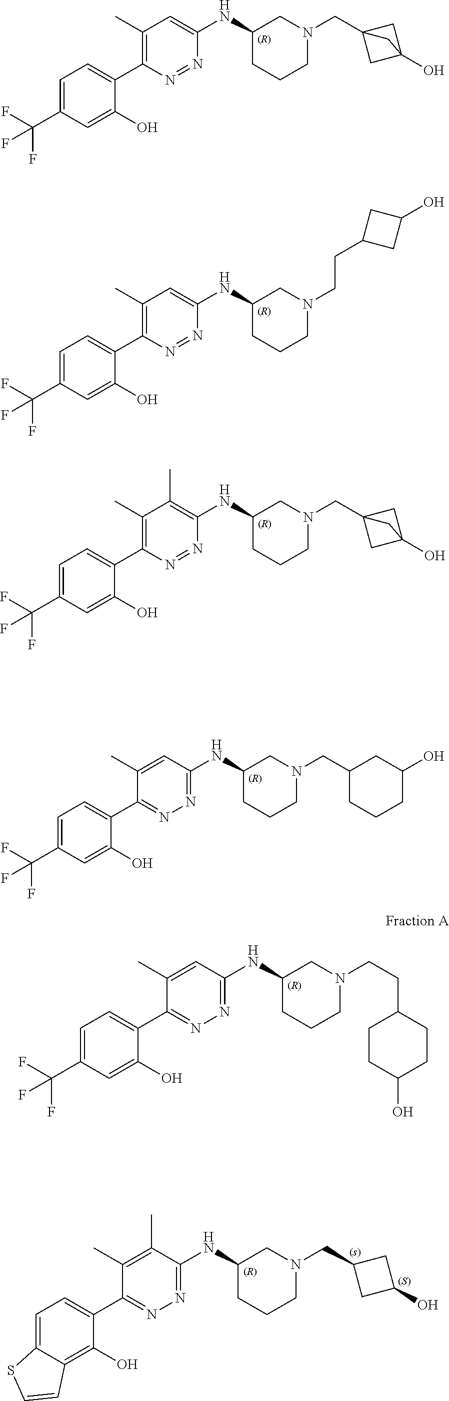

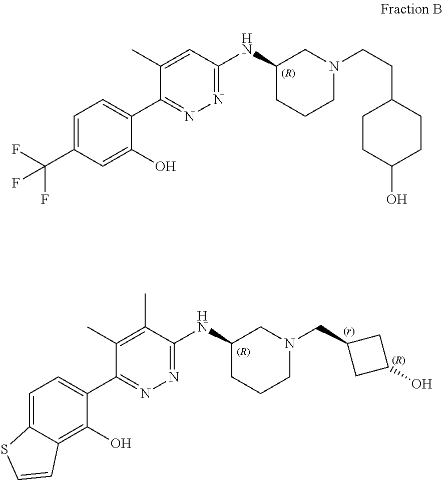

The present disclosure also provides a compound represented by the structure of Formula (1-VIII):

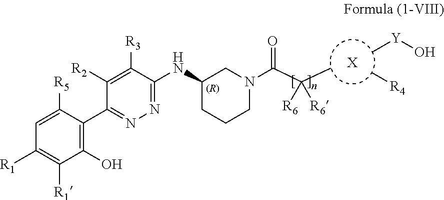

wherein ring

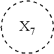

is a 4-11 membered ring, selected from cycloalkyl, substituted cycloalkyl, heterocycloalkyl, or substituted heterocycloalkyl, including spirocyclic and bicyclic cycloalkyl, substituted spirocyclic and bicyclic cycloalkyl rings, spirocyclic and bicyclic heterocycloalkyl, and substituted spirocyclic and bicyclic heterocycloalkyl rings, and R1 is selected from —CN, —H, —F, —CF3; or may form a thiophene ring together with R1′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃; R₆ and R₆′ are independently selected from —H, —F, C1-C6 alkyl, and —CF₃, or R₆ and R₆′ together with the atom they are bonded to form a C1-C6 cycloalkyl or C1-C6 heterocycloalkyl ring; n is selected from 1, 2, 3, and 4.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-VIII) is further represented by any one of the following:

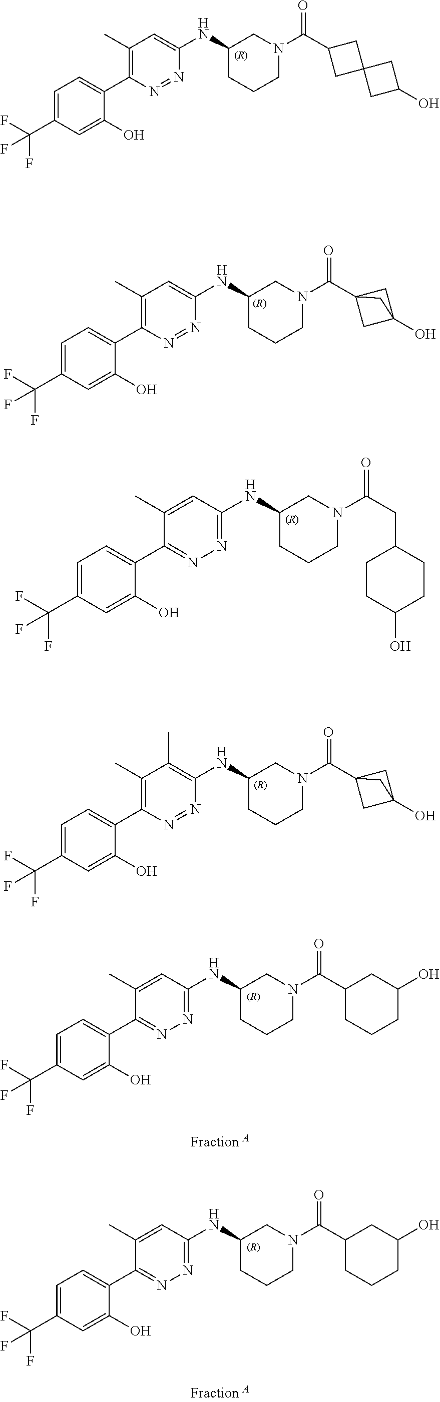

The present disclosure also provides a compound represented by the structure of Formula (1-IX):

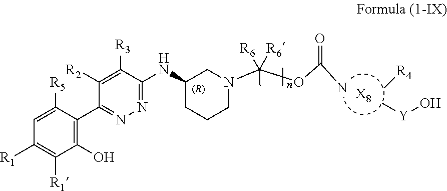

wherein ring

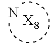

is a 4-11 membered nitrogen-containing heterocycloalkyl ring and substituted heterocycloalkyl ring, including spirocyclic and bicyclic nitrogen-containing heterocycloalkyl and substituted spirocyclic and bicyclic nitrogen-containing heterocycloalkyl rings, R₁ is selected from —CN, —H, —F, —CF₃; or may form a thiophene ring together with R₁′ (to make a benzothiophene with the sulfur atom attached to the carbon atom bearing R₁); R₁′ is H or may form a thiophene ring together with R₁ (to make a benzothiophene with the sulfur atom attached to the carbon atom bearing R₁); R₂ is selected from H, C1-C3 alkyl, —CF₃, or it forms a ring with R₃, a 5- or 6-membered aromatic or non-aromatic ring, all carbon or within O or N within the ring, and each of the C- or N-atoms within the 5- or 6-membered aromatic or non-aromatic ring is optionally substituted with -Me or —OH; R₃ is selected from H, C1-C3 alkyl, —CF₃, or it forms a ring with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from H, —F, C1-C3 alkyl, and —CF₃; R₅ is selected from H, Me and —CF₃; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 2, 3, and 4; Y is a bond (when Y is a bond, the —OH is attached directly to the ring) or Y is C1-C3 alkyl, optionally substituted with 1, 2, or 3 C1-C3 alkyl, or —CF₃.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (1-IX) is further represented by any one of the following:

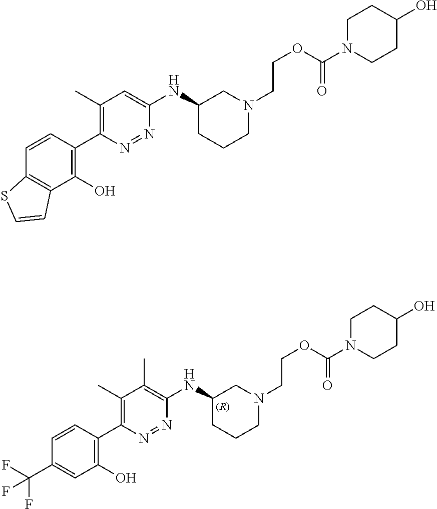

The present disclosure provides a compound represented by the structure of Formula (2-I):

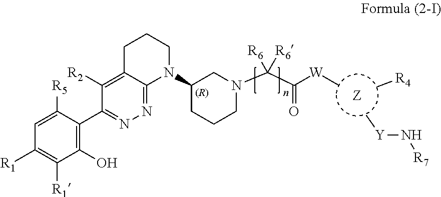

wherein ring

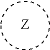

is a 4-11 membered ring, selected from cycloalkyl, substituted cycloalkyl, heterocycloalkyl, or substituted heterocycloalkyl, including spirocyclic and bicyclic cycloalkyl, substituted spirocyclic and bicyclic cycloalkyl, spirocyclic and bicyclic heterocycloalkyl, and substituted spirocyclic and bicyclic heterocycloalkyl, and where W is selected from NH, N-Me, N-Et and N-iPr, or is a bond; when W is a bond, the carbonyl group (C═O) is bonded to a nitrogen atom within ring

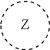

so as together they form an amide bond; R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 1, 2, 3, and 4; R₇ is selected from —H, C1-C6 alkyl, cycloalkyl, heterocycloalkyl,

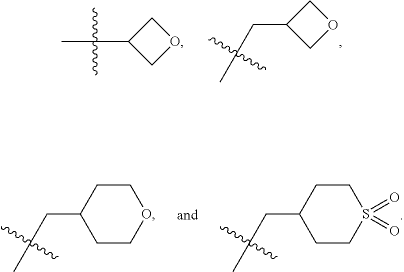 [SMILES: *C1COC1 ; *CC1COC1 ; *CC1CCOCC1 ; *CC1CCS(=O)(=O)CC1]

In some aspects of Formula (2-I), ring

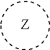

is a 4-11 membered ring, selected from cycloalkyl, substituted cycloalkyl, heterocycloalkyl or substituted heterocycloalkyl, including spirocyclic and bicyclic cycloalkyl, substituted spirocyclic and bicyclic cycloalkyl, spirocyclic and bicyclic heterocycloalkyl, and substituted spirocyclic and bicyclic heterocycloalkyl, and where W is selected from NH, N-Me, or is a bond; when W is a bond, the carbonyl group (C═O) is bonded to a nitrogen atom within ring

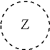

so as together they form an amide bond; R₁ is selected from —F, —CF₃; R₁′ is —H; R₂ is selected from —H, -Me, —CF₃, or it forms a ring together with R₃, a 5- or 6-membered non-aromatic ring, all carbon or with O within the ring; R₃ is selected from —H, -Me, or it forms a ring together with R₂, a 5- or 6-membered non-aromatic ring, all carbon or with O within the ring; R₄ is —H; Y I a bond; R₅ is selected from —H and -Me; R₆ and R₆′ are —H; n is 1; R₇ is —H.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-I) is further represented by any one of the following:

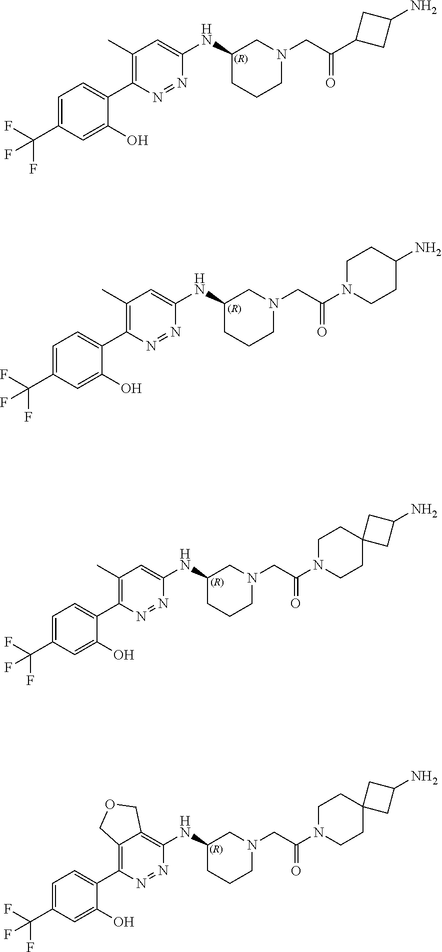

The present disclosure also provides a compound represented by the structure of Formula (2-II):

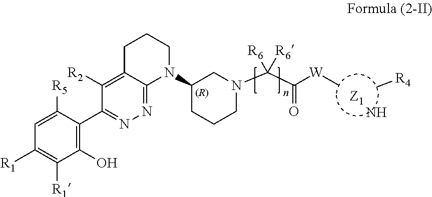

wherein ring

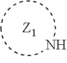

is a 4-11 membered nitrogen-containing ring, including heterocycloalkyl ring and substituted heterocycloalkyl ring, including spirocyclic and bicyclic nitrogen-containing heterocycloalkyl and substituted spirocyclic and bicyclic heterocycloalkyl, and where W is selected from NH, N-Me, N-Et and N-iPr, or is a bond; when W is a bond, the carbonyl group (C═O) is bonded to a nitrogen atom within ring

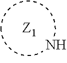

so as together they form an amide bond; R₁ is selected from —CN, —H, —F, —CF₃; or may form a thiophene ring together with R₁′ (to make a benzothiophene with the sulfur atom attached to the carbon atom bearing R₁); R₁′ is H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; R₅ is selected from —H, -Me, and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 1, 2, 3, and 4.

In some aspects of Formula (2-II), ring

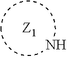

is a 4-11 membered nitrogen-containing heterocycloalkyl ring, including spirocyclic and bicyclic nitrogen-containing heterocycloalkyl, and where W is selected from NH, N-Me, or is a bond; when W is a bond, the carbonyl group (C═O) is bonded to a nitrogen atom within ring

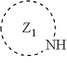

so as together they form an amide bond R₁ is selected from —F, —CF₃; R₁′ is —H; R₂ is selected from —H, -Me, —CF₃, or it forms a ring together with R₃, a 5- or 6-membered non-aromatic ring, all carbon or with O within the ring; R₃ is selected from —H, -Me, or it forms a ring together with R₂, a 5- or 6-membered non-aromatic ring, all carbon or with O within the ring; R₄ is —H or -Me; R₅ is selected from —H and -Me; R₆ and R₆′ are —H; n is 1.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-II) is further represented by any one of the following:

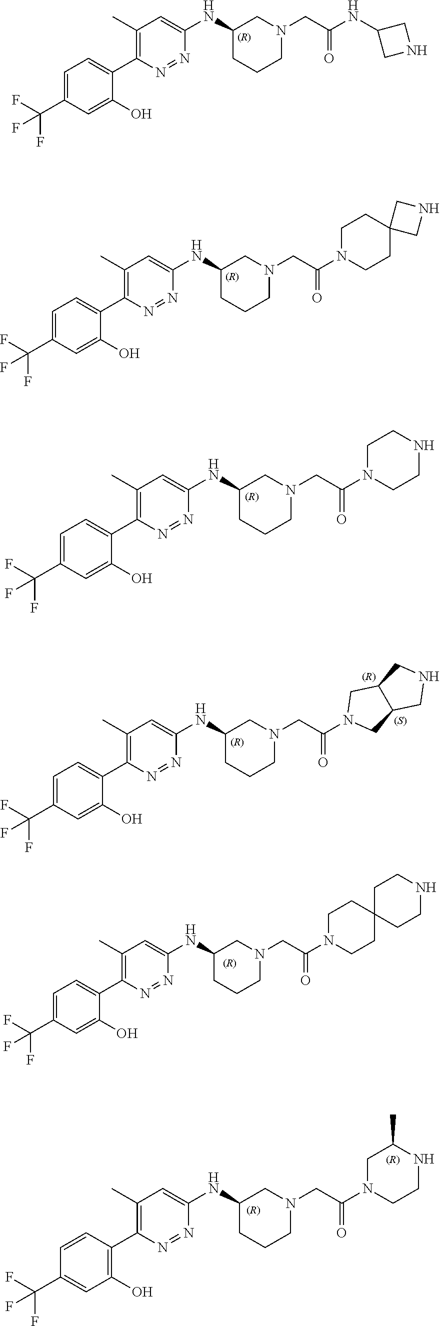

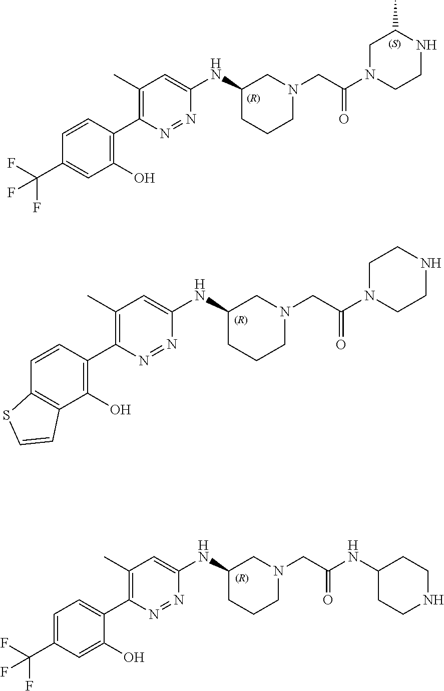

The present disclosure also provides a compound represented by the structure of Formula (2-III):

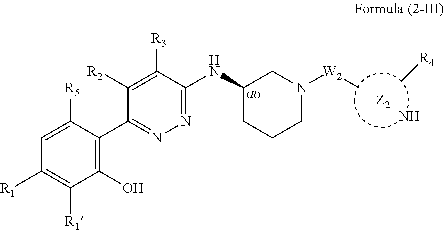

wherein ring

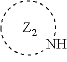

is a 4-11 membered ring, selected from heterocycloalkyl or substituted heterocycloalkyl, including spirocyclic and bicyclic heterocycloalkyl, and substituted spirocyclic and bicyclic heterocycloalkyl; R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; R₅ is selected from —H, -Me and —CF₃; where W₂ is selected from optionally-substituted —CH₂—, —CH₂CH₂—, and —CH₂CH₂CH₂—, or is a bond.

In some aspects of Formula (2-III), ring

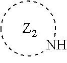

is a 4-9 membered nitrogen-containing heterocycloalkyl ring, including spirocyclic and bicyclic nitrogen-containing heterocycloalkyl, and where W₂ is selected from —CH₂—, —CH₂CH₂—, or is a bond; R₁ is selected from —F, —CF₃; R₁′ is —H; R₂ is selected from —H, -Me, —CF₃, or it forms a ring with R₃, a 5- or 6-membered non-aromatic ring, all carbon or with O within the ring; R₃ is selected from —H, -Me, or it forms a ring with R₂, a 5- or 6-membered non-aromatic ring, all carbon or with O within the ring; R₄ is —H or -Me; R₅ is selected from —H and -Me.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-III) is further represented by any one of the following:

In several embodiments, the compound of Formula (2-III) is further represented by any one of the following:

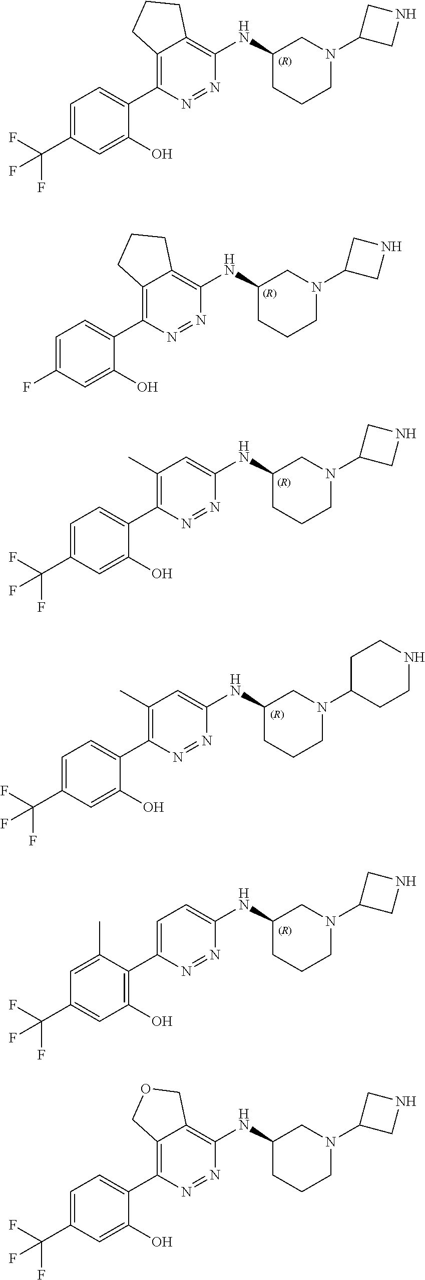

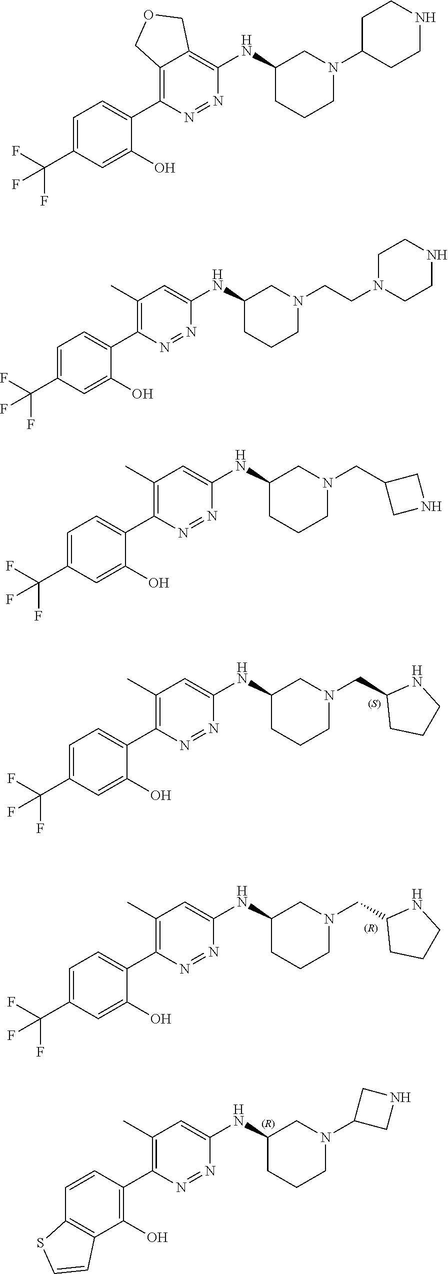

The present disclosure also provides a compound represented by the structure of Formula (2-IV):

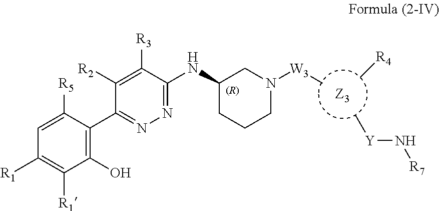

wherein ring

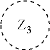

is a 4-11 membered ring, selected from cycloalkyl, substituted cycloalkyl, heterocycloalkyl or substituted heterocycloalkyl, including spirocyclic and bicyclic cycloalkyl, substituted spirocyclic and bicyclic cycloalkyl, spirocyclic and bicyclic heterocycloalkyl, and substituted spirocyclic and bicyclic heterocycloalkyl; R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; Y is a bond (when Y is a bond, the —OH is attached directly to the ring) or Y is C1-C3 alkyl, optionally substituted with 1, 2, or 3 C1-C3 alkyl, or —CF₃; R₅ is selected from —H, -Me, and —CF₃; where W₃ is selected from optionally-substituted —CH₂—, —CH₂CH₂—, and —CH₂CH₂CH₂—, or is a bond; R₇ is selected from —H, C1-C6 alkyl, cycloalkyl, heterocycloalkyl,

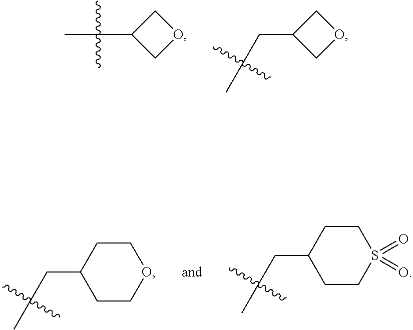 [SMILES: *C1COC1 ; *CC1COC1 ; *CC1CCOCC1 ; *CC1CCS(=O)(=O)CC1]

In some aspects of Formula (2-IV), ring

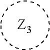

is a 4-9 membered cycloalkyl ring or heterocycloalkyl ring, and where W₃ is selected from —CH₂— and —CH₂CH₂—, or is a bond; R₁ is selected from —F and —CF₃; R₁′ is —H; R₂ is selected from —H, -Me, and —CF₃, or it forms a ring with R₃, a 5- or 6-membered aromatic or non-aromatic ring, all carbon or with O within the ring; R₃ is selected from —H and -Me, or it forms a ring with R₂, a 5- or 6-membered aromatic or non-aromatic ring, all carbon or with O within the ring; R₄ is —H or -Me; Y is a bond; R₅ is selected from —H and -Me; R₇ is —H.

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-IV) is further represented by any one of the following:

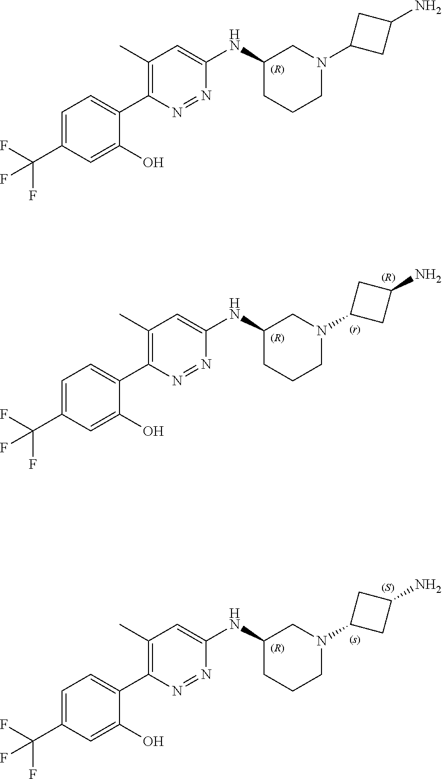

The present disclosure also provides a compound represented by the structure of Formula (2-V):

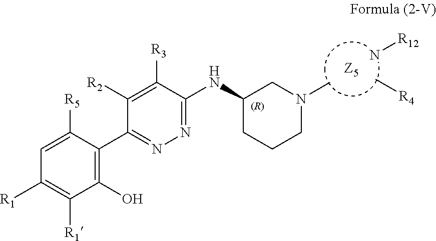

wherein ring

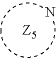

is azetidine, pyrrolidine, piperidine or azepane; R1 is selected from —CN, —H, —F, —CF3; or may form a thiophene ring together with R1′ (to make a benzothiophene with the sulfur atom attached to the carbon atom bearing R1); R1′ is —H or may form a thiophene ring together with R1 (to make a benzothiophene with the sulfur atom attached to the carbon atom bearing R1); R2 is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₄ is selected from —H, —F, C1-C3 alkyl, and —CF₃; R₅ is selected from —H, -Me and —CF₃; R₁₂ is selected from:

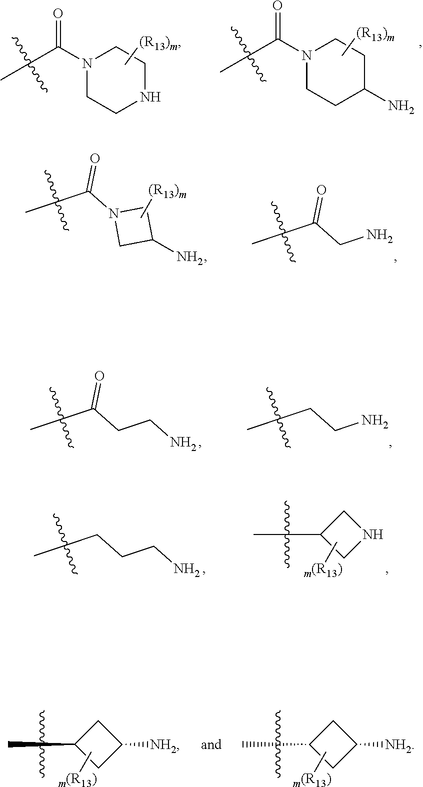

wherein m is 1, 2, or 3, with each R₁₃ independently selected from —H, C1-C3 alkyl, and —CF₃. Two R₁₃ groups may connect to form a cyclopropyl or cyclobutyl ring.

In some aspects of Formula (2-V), ring

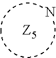

is azetidine or piperidine; R₁ is selected from —F, —CF₃; R₁′ is —H; R₂ is selected from —H, -Me, and —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₄ is —H or -Me; R₅ is selected from —H and -Me; R₁₂ is selected from:

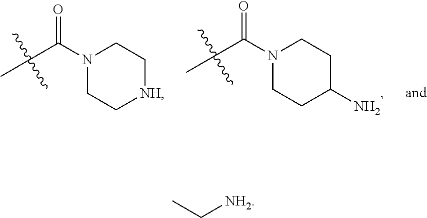

In some embodiments, R₄ is present as hydrogen or otherwise absent from the ring. In some embodiments, R₄ is present and substituted on any position of the ring as indicated above. In some embodiments, R₄ is present twice and substituted on any position of the ring as indicated above. In some embodiments, R₄ is present three, four, five or six times, and substituted on any position of the ring as indicated above.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-V) is further represented by any one of the following:

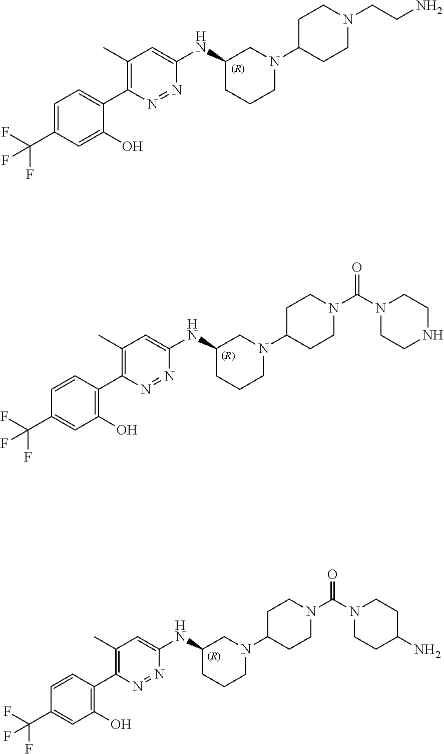

The present disclosure also provides a compound represented by the structure of Formula (2-VI):

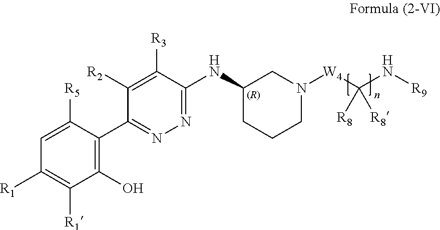

where R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring together with R₁ (to make a benzothiophene with the sulfur atom attached to the carbon atom bearing R₁); R₂ is selected from —H, C1-C3 alkyl, and —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, and —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₅ is selected from —H, -Me and —CF₃; W₄ is selected from —CH₂—, —CH₂CH₂—,

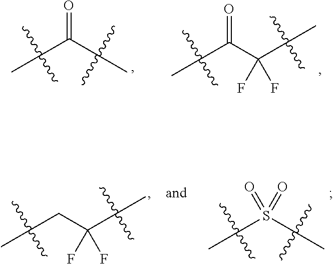

R₈ and R₈′ are independently selected from —H, C1-C5 alkyl (including cycloalkyl), and —CF₃; R₉ is selected from —H, C1-C6 alkyl, C1-C6 cycloalkyl, heterocycloalkyl,

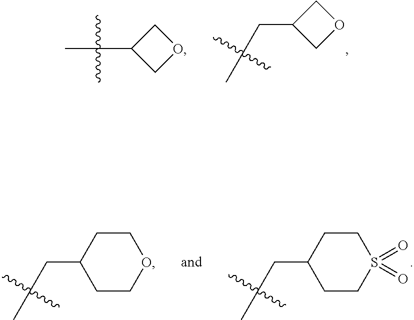 [SMILES: *C1COC1 ; *CC1COC1 ; *CC1CCOCC1 ; *CC1CCS(=O)(=O)CC1]

In some aspects of Formula (2-VI), R₁ is selected from —F, —CF₃; R₁′ is —H; R₂ is selected from —H, -Me, —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₅ is selected from —H and -Me; W₄ is selected from —CH₂— or

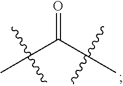 [SMILES: *C(*)=O]

R₈ and R₈′ are —H; n is 1; R₉ is —H.

In some aspects of Formula (2-VI), R₁ is selected from —F, —CF₃; R₁′ is —H; R₂ is selected from —H, -Me, —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₅ is selected from —H and -Me; W₄ is selected from —CH₂— or

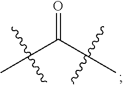 [SMILES: *C(*)=O]

R₈ and R₈′ are —H; n is 1; R₉ is selected from

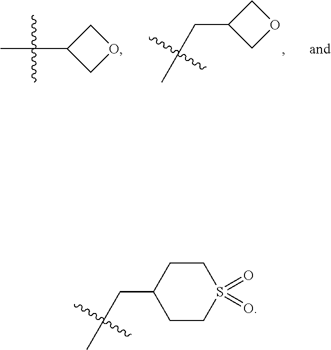 [SMILES: *C1COC1 ; *CC1COC1 ; *CC1CCS(=O)(=O)CC1]

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In several embodiments, the compound of Formula (2-VI) is further represented by any one of the following:

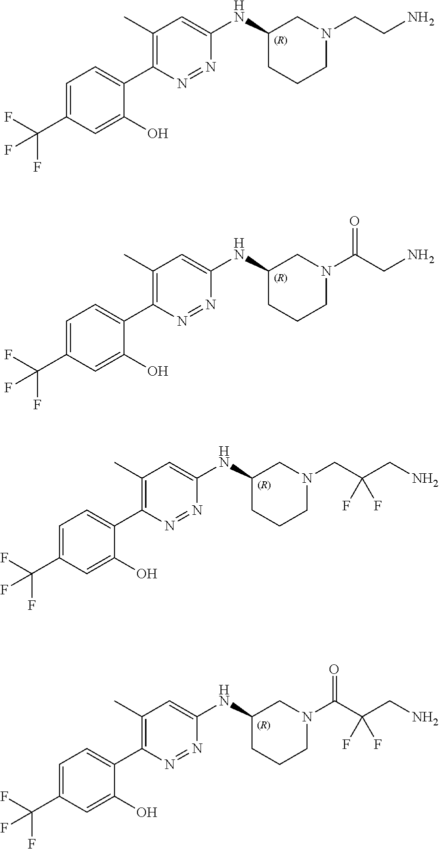

In some embodiments, the compound of Formula (2-VI) is further represented by any one of the following:

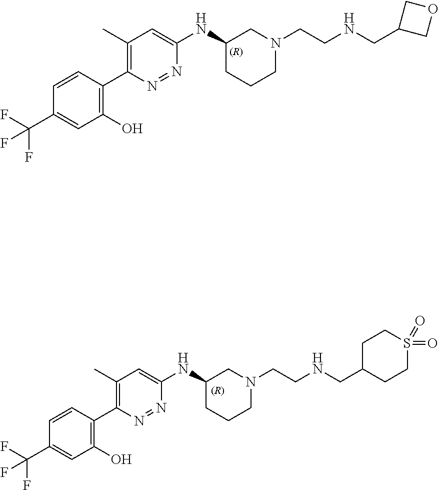

The present disclosure also provides a compound represented by the structure of Formula (2-VII):

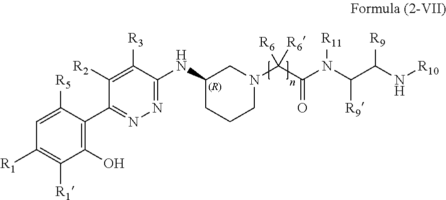

where R₁ is selected from —CN, —H, —F, —CF₃; or may form a thiophene ring together with R₁′ (to make a benzothiophene with the sulfur atom attached to the carbon atom bearing R₁); R₁′ is —H or may form a thiophene ring with R₁ (to make a benzothiophene with the sulfur atom attached to the carbon atom bearing R₁); R₂ is selected from —H, C1-C3 alkyl, —CF₃, or it forms a ring together with R₃, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₃ is selected from —H, C1-C3 alkyl, —CF₃, or it forms a ring together with R₂, a 5- or 6-membered aromatic or non-aromatic ring, all carbon or with O or N within the ring, and each of the C- or N-atoms within the 5- or 6-membered aromatic or non-aromatic ring is optionally substituted with -Me or —OH; R₅ is selected from —H, -Me and —CF₃; R₆ and R₆′ are independently selected from —H, —F, C1-C6 alkyl or cycloalkyl, and —CF₃, or R₆ and R₆′ together with the atom they are bonded to form a C1-C6 cycloalkyl or C1-C6 heterocycloalkyl ring; n is selected from 1, 2, 3, and 4; R₉ and R₉′ are independently selected from —H, C1-C5 alkyl (including cycloalkyl), —CF₃, and R₉ and R₉′ together with the atoms that they are bonded to form a (substituted) C3-C6 cycloalkyl or (substituted) C3-C6 heterocycloalkyl ring; R₁₀ is selected from —H, C1-C7 alkyl (including cycloalkyl and methylcycloalkyl),

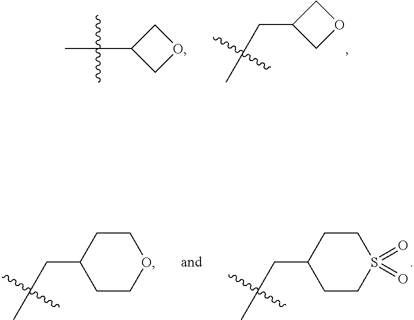 [SMILES: *C1COC1 ; *CC1COC1 ; *CC1CCOCC1 ; *CC1CCS(=O)(=O)CC1]

R₁₁ is selected from —H, C1-C7 alkyl (including cycloalkyl and methylcycloalkyl).

In some aspects of Formula (2-VII), R₁ is selected from —F, —CF₃; R₁′ is —H; R₂ is selected from —H, -Me, —CF₃, or it forms a ring together with R₃, a 5- or 6-membered aromatic or non-aromatic ring, all carbon or with O within the ring; R₃ is selected from —H, -Me, or it forms a ring together with R₂, a 5- or 6-membered aromatic or non-aromatic ring, all carbon or with O within the ring; R₅ is selected from —H and -Me; R₆ and R₆′ are —H; n is 1; R₉ and R₉′ are —H; R₁₀ is —H; R₁₁ is selected from —H or -Me.

In some embodiments, R₁ forms a thiophene ring together with R₁′ (which then make a benzothiophene with the ring R₁ and R₁′ is attached to). At least two isomeric thiophenes are possible for this structure—where S is attached to the carbon atom bearing R₁ and where S is attached to the carbon atom bearing R₁′. In some embodiments, a preferred benzothiophene substructure may have the sulfur atom attached to the carbon atom bearing R₁.

In one embodiment, the compound of Formula (2-VII) is further represented by the following:

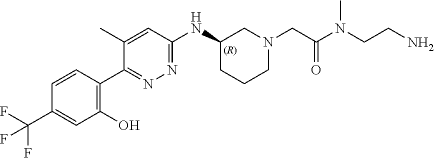

### Method of Preparation

The compounds disclosed herein may be synthesized by methods described below, or by modification of these methods. Ways of modifying the methodology include, among others, temperature, solvent, reagents etc., known to those skilled in the art and are part of routine reaction modification and optimization. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more stereogenic centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents or catalysts well-known in the art. Alternatively, racemic mixtures of such compounds can be separated using, for example, chiral column chromatography, chiral resolving agents and the like.

The starting materials for the following reactions are generally known or commercial compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The methods disclosed herein may include using standard organic synthesis techniques to construct compounds of the general Formula (A), as shown in Scheme 1-1. Ar is an appropriately substituted phenol or protected phenol (if a protected phenol is used, a deprotection step will be needed after the amide bond formation). Alternatively, compounds of general Formula (A) can be made starting with Cl instead of Ar and making the final compound via a Suzuki-coupling with the appropriate Ar-boronic acid in the last step. The reagents shown in Scheme 1-1 are either commercially available or can be readily synthesized by the skilled artisan using well known literature methods.

General Scheme 1-1 shows a cyclic secondary amino alcohol, such as a substituted or unsubstituted piperidinol, pyrrolidinol, azetidinol, or a spirocyclic secondary amino alcohol. However, the same approach can be taken with primary amino alcohols, such as 4-aminocyclohexanol or 4-aminocyclobutanol, to make compounds of the invention.

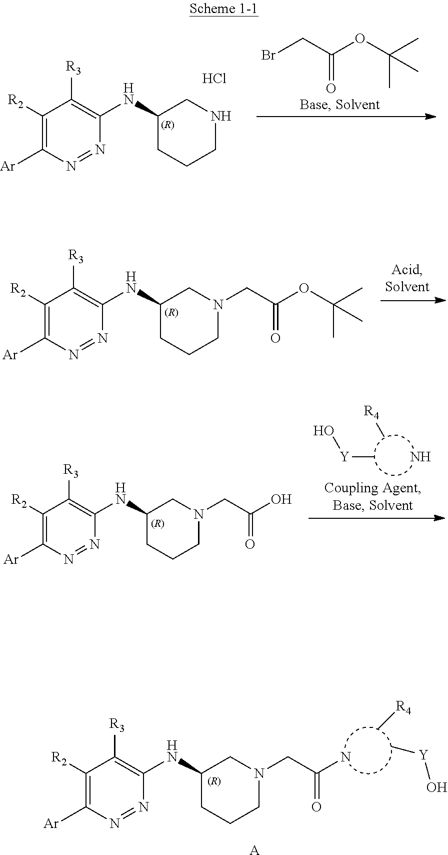

Compounds of general formula (B) may be made via general Scheme 1-2. The phenol on Ar may need to be appropriately protected in the first step of Scheme 2. Alternatively, compounds of general Formula (B) can be made starting with Cl instead of Ar and making the final compound via a Suzuki-coupling with the appropriate Ar-boronic acid in the last step. The reagents shown in Scheme 1-2 are either commercially available or can be readily synthesized by the skilled artisan using well known literature methods.

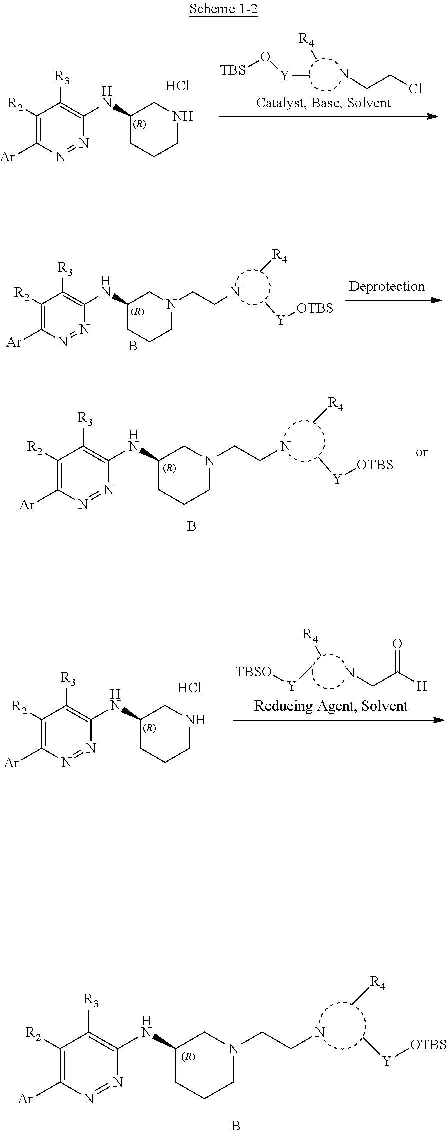

Compounds of general Formula (C) may be made via general Scheme 1-3. Ar is an appropriately substituted phenol or protected phenol or protected phenol (if a protected phenol is used, a deprotection step will be needed after the amide bond formation). Alternatively, compounds of general Formula (C) can be made starting with Cl instead of Ar and making the final compound via a Suzuki-coupling with the appropriate Ar-boronic acid in the last step. The reagents shown in Scheme 1-3 are either commercially available or can be readily synthesized by the skilled artisan using well known literature methods.

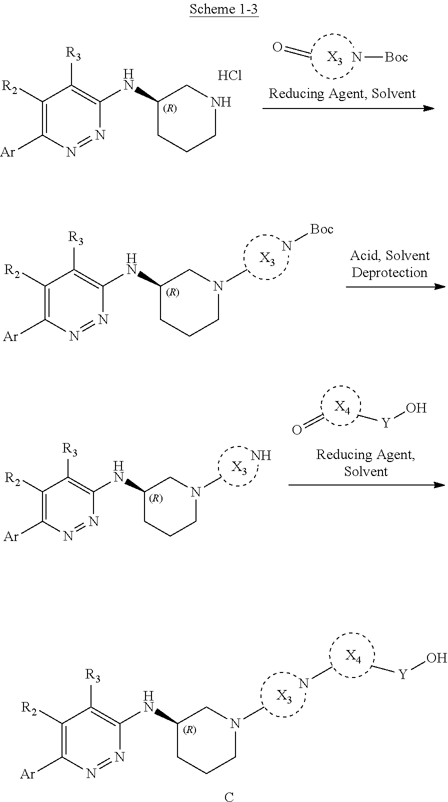

Compounds of general Formula (D) may be made via general Scheme 1-4. Ar is an appropriately substituted phenol or protected phenol or protected phenol (if a protected phenol is used, a deprotection step will be needed after the amide bond formation). Alternatively, compounds of general Formula (D) can be made starting with Cl instead of Ar and making the final compound via a Suzuki-coupling with the appropriate Ar-boronic acid in the last step. The reagents shown in Scheme 1-4 are either commercially available or can be readily synthesized by the skilled artisan using well known literature methods.

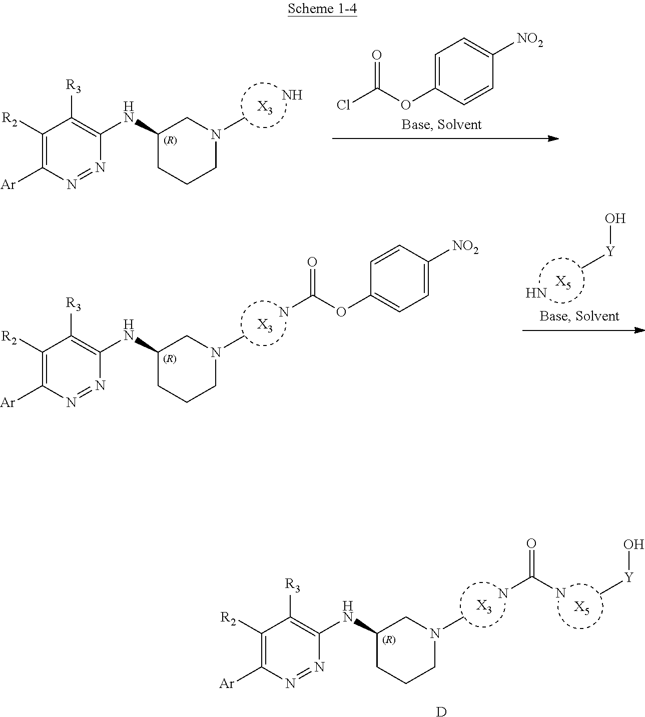

Compounds of general Formula (E) may be made via general Scheme 1-5. Ar is an appropriately substituted phenol or protected phenol or protected phenol (if a protected phenol is used, a deprotection step will be needed after the amide bond formation). Alternatively, compounds of general Formula (E) can be made starting with Cl instead of Ar and making the final compound via a Suzuki-coupling with the appropriate Ar-boronic acid in the last step. The reagents shown in Scheme 1-5 are either commercially available or can be readily synthesized by the skilled artisan using well known literature methods.

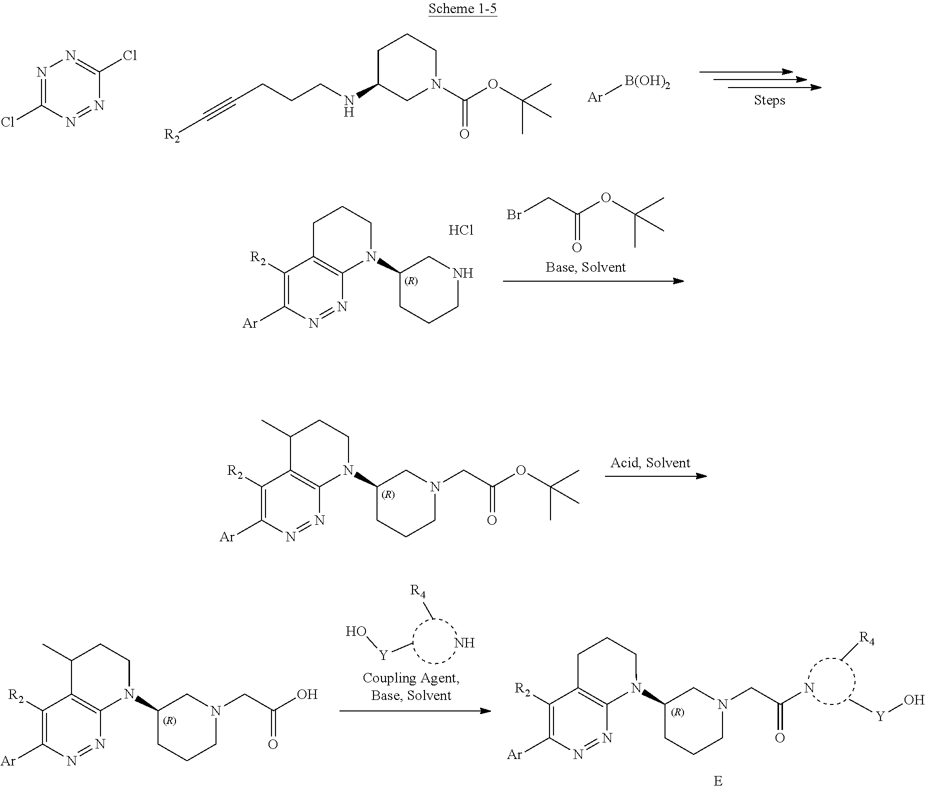

The methods disclosed herein may include using standard organic synthesis techniques to construct compounds of the general Formula (A) and general Formula (B), as shown in Scheme 2-1 and Scheme 2-2. Ar is an appropriately substituted phenol or protected phenol (if a protected phenol is used, a deprotection step will be needed to liberate the protected phenolic hydroxy group at an appropriate synthetic step.). Alternatively, compounds of general Formula (A) and (B) can be made starting with Cl instead of Ar and making the final compound via a Suzuki-coupling with the appropriate Ar-boronic acid in the last step. The reagents shown in Scheme 2-1 are either commercially available or can be readily synthesized by the skilled artisan using well known literature methods.

General Scheme 2-1 and Scheme 2-2 show a protected diamine coupling to a key acid intermediate to form an amide, then deprotection of the terminal amine to form a final product.

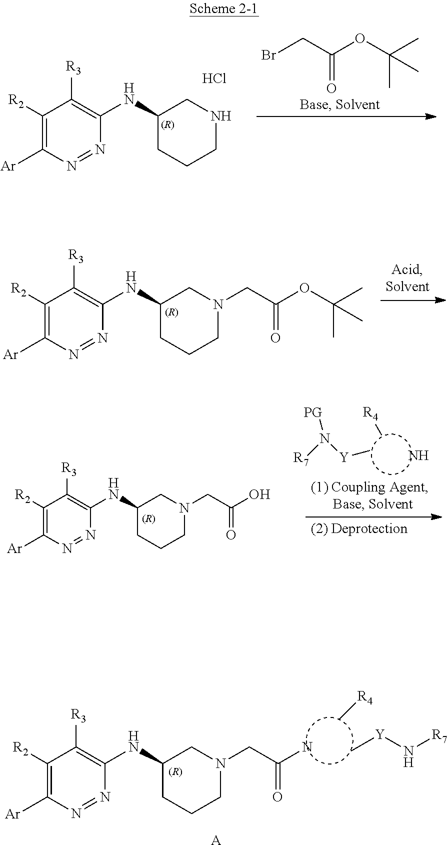

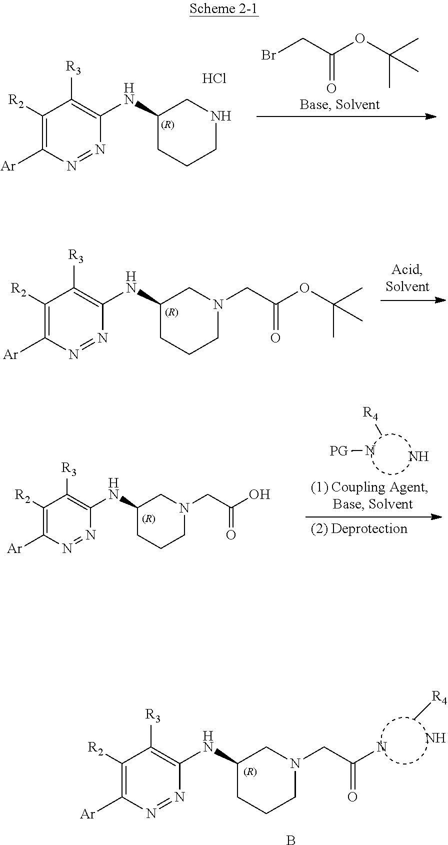

Compounds of general Formula (C) and general Formula (D) may be made via general Scheme 2-3. Ar is an appropriately substituted phenol or protected phenol or protected phenol (if a protected phenol is used, a deprotection step will be needed to liberate the protected phenolic hydroxy group at an appropriate synthetic step). Alternatively, compounds of general Formula (C) can be made starting with Cl instead of Ar and making the final compound via a Suzuki-coupling with the appropriate Ar-boronic acid in the last step. Removal of a protecting group may be required to obtain compounds of Formula (D). The reagents shown in Scheme 2-3 are either commercially available or can be readily synthesized by the skilled artisan using well known literature methods.

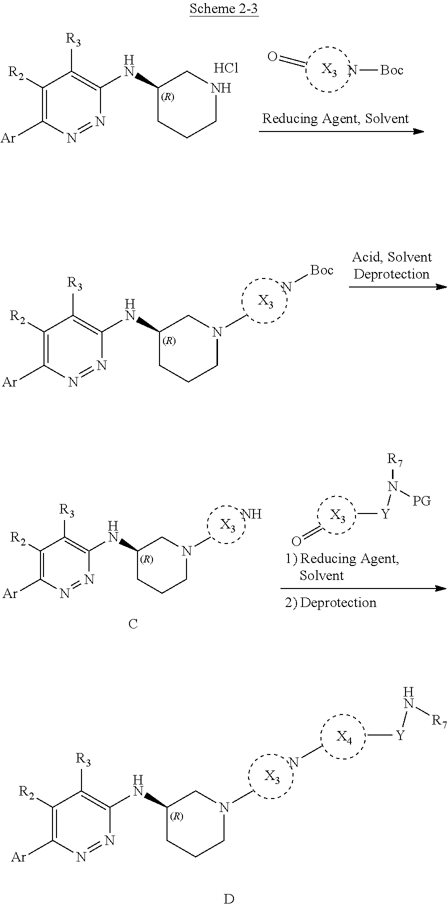

Compounds of general Formula (E) may be made via general Scheme 2-24. Ar is an appropriately substituted phenol or protected phenol or protected phenol (if a protected phenol is used, a deprotection step will be needed to liberate the protected phenolic hydroxy group at an appropriate synthetic step.). Alternatively, compounds of general Formula (E) can be made starting with Cl instead of Ar and making the final compound via a Suzuki-coupling with the appropriate Ar-boronic acid in the last step. The reagents shown in Scheme 2-4 are either commercially available or can be readily synthesized by the skilled artisan using well known literature methods.

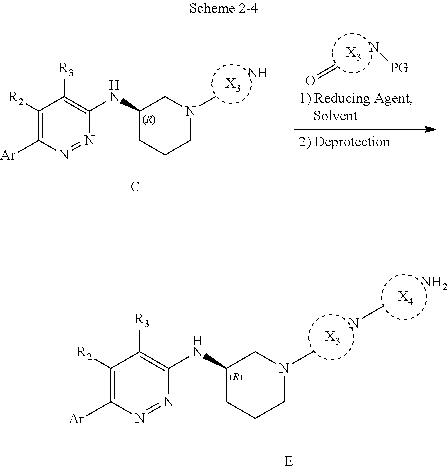

The above example schemes are provided for the guidance of the reader, and collectively represent example methods for making the compounds encompassed herein. Furthermore, other methods for preparing compounds described herein will be readily apparent to the person of ordinary skill in the art considering the following reaction schemes and examples.

### Method of Treatment

Provided herein are methods that are capable of inhibiting NLRP3 family proteins and NLRP3 inflammasome function and/or IL-1ß secretion. Various embodiments of these methods include compounds having the structures described herein.

The present disclosure provides a method of preventing, treating, or ameliorating one or more diseases in a subject. In several embodiments, the method includes administering to a subject in need thereof at least one compound having a structure as described elsewhere herein. In several embodiments, the method includes administering to a subject in need thereof a pharmaceutically acceptable salt of at least one compound having a structure as described elsewhere herein.

In several embodiments, the disease is characterized by a disease progression that comprises the activity of at least one member of the IL-1 family of cytokines. In several embodiments, the disease progression comprises the activity of at least one IL-1 cytokine with agonistic activity, antagonistic activity, anti-inflammatory activity, and any combination of the foregoing. In several embodiments, the disease progression comprises the activity of at least one IL-1 cytokine with agonistic activity. In several embodiments, the disease progression comprises the activity of at least one of IL-1α, IL-1β, IL-18, IL-33, IL-36α, IL-36β, IL-36γ, IL-1Ra, IL-36Ra, IL-38, and IL-37. In several embodiments, the disease progression comprises the activity of at least one of IL-1α, IL-1β, IL-18, IL-33, IL-36α, IL-36β, IL-36γ. In several embodiments, the disease progression comprises the activity of IL-1b. In several embodiments, the disease progression comprises the activity of IL-18. In several embodiments, the disease progression comprises the activity of IL-1b and IL-18.

In several embodiments, the disease is at least one chronic inflammatory disorder. In several embodiments, the disease is characterized by a disease progression pathology that comprises the activity of NLRP3 inflammasome. In several embodiments, the NLRP3 inflammasome comprises at least one mutation.

As a non-limiting example, age-related macular degeneration (AMD), both wet and dry types, involves the deterioration of the central part of the retina, leading to vision loss. NLRP3 inflammasome activation may contribute to the inflammation and neovascularization seen in AMD. Similarly, diabetic macular edema (DME) and diabetic retinopathy (DR) are complications of diabetes where high blood sugar levels damage the retinal blood vessels, and inflammation plays a significant role in their progression.

Diseases like glaucoma, characterized by increased pressure in the eye leading to optic nerve damage, and retinopathy, involving damage to the retinal blood vessels, are also associated with inflammatory responses mediated by the NLRP3 inflammasome. Dry eye disease (DED) and bacterial keratitis, an infection of the cornea, involve inflammation where NLRP3 might play a role in the response to infection and cellular stress.

Inflammatory and autoimmune diseases like Behcet's syndrome, systemic lupus erythematosus (SLE), and rheumatoid arthritis can have ocular manifestations, including uveitis (inflammation of the middle layer of the eye) and retinal vasculitis (inflammation of the retinal vessels). These conditions are often marked by an overactive immune response, where controlling NLRP3 inflammasome activation may manage inflammation and prevent tissue damage. Targeting the NLRP3 inflammasome for patients experiencing, or at risk of experiencing, the various diseases mentioned above may reduce inflammation, slow disease progression, preserve vision, and improve quality of life by addressing one of the underlying mechanisms contributing to these eye conditions.

In several embodiments, the disease is selected from the group consisting of, for example, age-related macular degeneration (wet and dry) (AMD), atrophic macular degeneration, bacterial endophthalmitis, bacterial infections of the eye, bacterial corneal ulcers incl, but not limited to Pseudomonas aeruginosa and Streptococcus pneumoniae corneal ulcers, bacterial keratitis incl, but not limited to Pseudomonas aeruginosa keratitis, Behcet's syndrome, cataract, choroidal neovascularization, CMV-retinitis, chronic eye disease, delayed diabetic corneal wound healing and nerve degeneration, diabetic macular edema (DME), diabetic retinopathy (DR) incl, but not limited to proliferative diabetic retinopathy, conjunctivitis, corneal allograft rejection incl, but not limited to corneal graft failure (CGF), corneal edema, dry eye disease (DED), Graves' disease, Fuchs endothelial corneal dystrophy, fungal endophthalmitis, fungal keratitis incl, but not limited to Candida albicans keratitis, fungal infections of the eye, glaucoma (acute and non-acute), geographic atrophy (GA), inflammatory eye disease, keratitis fugax hereditaria (KFH), LPS-induced ocular inflammation, Mooren's ulcer (MU), neuronal death in retinal ischemia/reperfusion injury, ocular hypertension, ocular inflammation associated with CAPS ((CAPS that include familial cold autoinflammatory syndrome), ocular manifestations of rheumatoid arthritis and systemic lupus erythematosus (SLE), ocular limbal squamous cell carcinoma, optic neuritis, parasitic infections of the eye, pathologic neovascularization, excitatory retinal overstimulation, lipofuscin and A2E-mediated oxidative damage incl, but not limited to ROS/oxidative stress reduction, retinal ganglion cell (RGC) dysfunction and death in response to ocular hypertension (OHT)-induced stress (OHT-glaucoma), retinoblastoma, retinitis, retinal vasculitis, retinal vein occlusion (RVO), Sjörgen's syndrome, sterile corneal inflammation, stroke induced retinal injury in diabetes, traumatic optic neuropathy/trauma (optic nerve crush) incl, but not limited to progressive optic neuropathy, ulcerative keratitis, uveal melanoma, uveitis (anterior/intermediate/posterior, pan-uveitis), and viral infections of the eye incl. but not limited to inflammatory herpetic disease.

In several embodiments, the disease is selected from the group consisting of, for example age-related macular degeneration (wet and dry) (AMD), atrophic macular degeneration, bacterial keratitis, Behcet's syndrome, choroidal neovascularization, chronic eye disease, diabetic macular edema (DME), diabetic retinopathy (DR), dry eye disease (DED), glaucoma (acute and non-acute), geographic atrophy (GA), inflammatory eye disease, ocular inflammation associated with CAPS, ocular manifestations of rheumatoid arthritis and systemic lupus erythematosus (SLE), retinitis, retinal vasculitis, retinal vein occlusion (RVO), progressive optic neuropathy, and uveitis (anterior/intermediate/posterior, pan-uveitis).

Consistent with the disclosure, compounds of the disclosure may be used for treating, preventing or ameliorating a disease affecting the eye. In several embodiments, the disease is selected from the group consisting of, for example, age-related macular degeneration (wet and dry) (AMD), atrophic macular degeneration, bacterial keratitis, Behcet's syndrome, choroidal neovascularization, chronic eye disease, diabetic macular edema (DME), diabetic retinopathy (DR), dry eye disease (DED, glaucoma (acute and non-acute), geographic atrophy (GA), and retinopathy.

In several embodiments, the disease is selected from the group consisting of, for example, inflammatory eye disease, ocular inflammation associated with cryopyrin-associated periodic syndrome (CAPS), ocular manifestations of rheumatoid arthritis and systemic lupus erythematosus (SLE), retinitis, retinal vasculitis, retinal vein occlusion (RVO), progressive optic neuropathy, and uveitis (anterior/intermediate/posterior, pan-uveitis).

In some embodiments, the disease is age-related macular degeneration (AMD). In some embodiments, the disease is atrophic macular degeneration. In some embodiments, the disease is bacterial keratitis. In some embodiments, the disease is Behcet's syndrome. In some embodiments, the disease is choroidal neovascularization. In some embodiments, the disease is chronic eye disease. In some embodiments, the disease is diabetic macular edema (DME). In some embodiments, the disease is diabetic retinopathy (DR). In some embodiments, the disease is dry eye disease (DED).

In some embodiments, the disease is glaucoma, both acute and non-acute. In some embodiments, the disease is geographic atrophy (GA). In some embodiments, the disease is retinopathy. In some embodiments, the disease is inflammatory eye disease. In some embodiments, the disease is associated with ocular inflammation from cryopyrin-associated periodic syndrome (CAPS). In some embodiments, the disease involves ocular manifestations of rheumatoid arthritis and systemic lupus erythematosus (SLE). In some embodiments, the disease is retinitis. In some embodiments, the disease is retinal vasculitis. In some embodiments, the disease is retinal vein occlusion (RVO). In some embodiments, the disease is progressive optic neuropathy. In some embodiments, the disease is uveitis, including anterior, intermediate, posterior, or pan-uveitis.

In some embodiments, due to activity towards the NLRP3 inflammasome, the compounds of the disclosure are expected to have significant biological activity against a range of systemic diseases. For instance, in diseases like gout and atherosclerosis, where inflammation is a primary factor, compounds of the disclosure may reduce symptoms and progression by modulating the inflammatory response. Accordingly, compounds that inhibit/stimulate NLRP3 inflammasome activation are sought after for their potential in treating a variety of systemic indications. The NLRP3 inflammasome plays a role in the human immune response, and its dysregulation is implicated in numerous diseases characterized by chronic inflammation and immune system dysfunction.

In some embodiments, compounds and methods of the disclosure may be used to treat or ameliorating systemic indications for NLRP3 activation compounds including autoinflammatory diseases. Conditions such as Cryopyrin-Associated Periodic Syndromes (CAPS) and Familial Mediterranean Fever are directly related to NLRP3 dysregulation. Similarly, autoimmune diseases like Rheumatoid Arthritis, Systemic Lupus Erythematosus (SLE), and Multiple Sclerosis, where chronic inflammation is sustained through immune response, may benefit from such compounds.

Metabolic disorders, notably Type 2 Diabetes and obesity, are also potential targets for NLRP3 inhibitors. These conditions are associated with chronic low-grade inflammation where NLRP3 might contribute to insulin resistance and metabolic complications. In the realm of neurodegenerative diseases, conditions like Alzheimer's Disease and Parkinson's Disease might benefit from NLRP3 inhibition, given the role of inflammation in neurodegeneration.

Cardiovascular diseases, including atherosclerosis, involve chronic inflammation where NLRP3 might be implicated in plaque formation and cardiac dysfunction. Liver diseases such as Non-alcoholic Steatohepatitis (NASH) and alcoholic liver disease also involve NLRP3-mediated inflammation. Similarly, kidney diseases like Acute Kidney Injury and Diabetic Nephropathy might be influenced by NLRP3 activation. Lastly, pulmonary diseases such as Asthma, Chronic Obstructive Pulmonary Disease (COPD), and Idiopathic Pulmonary Fibrosis, which have an inflammatory component, may also be viable targets for compounds that modulate NLRP3 activity.

Without being bound to a single theory of operation, NLRP3 inflammasome activation has been linked to various inflammasome-related diseases/disorders, immune diseases, inflammatory diseases, auto-immune diseases, and auto-inflammatory diseases. Nonlimiting examples of inflammasome-related diseases include atherosclerosis and cardiovascular risk (e.g., cardiovascular risk reduction (CvRR), hypertension); autoinflammatory fever syndrome cryopyrin-associated periodic syndrome (e.g., CAPS); cancer (e.g., colon cancer, lung cancer, myeloproliferative neoplasms, leukemias, myelodysplastic syndromes (MDS), myelofibrosis); chronic liver disease; gout; hidradenitis suppurativa; hyperoxaluria; neuroinflammation-related disorders (e.g., multiple sclerosis, brain infection, acute injury, neurodegenerative diseases, Alzheimer's disease); nonalcoholic steatohepatitis (NASH); pseudogout (chondrocalcinosis); sickle cell disease; Type I/Type II diabetes and related complications (e.g., nephropathy, retinopathy); and wound healing and scar formation.

In several embodiments, the disease is selected from the group consisting of, for example, acute or chronic arthropathy; alcoholic liver disease; alcoholic steatohepatitis; autoinflammatory fever syndrome such as cryopyrin-associated periodic syndrome (CAPS); chronic liver disease; diabetic nephropathy which is a kidney related complication of diabetes (Type 1, Type 2, and mellitus diabetes); gout; hemodialysis related inflammation; hypertensive nephropathy; inflammatory arthritis related disorders such as osteoarthritis and rheumatoid arthritis; kidney related diseases such as hyperoxaluria and lupus nephritis; liver related diseases/disorders such as viral hepatitis and non-alcoholic steatohepatitis (NASH); pseudogout (chondrocalcinosis); sickle cell disease; systemic lupus erythematosus (SLE).

Additionally, the increased production of IL-1b and IL-18 by the NLRP3 inflammasome has been linked to the onset and progression of various diseases such as neuro inflammation related disorders, e.g. brain infection, acute injury, multiple sclerosis, Alzheimer's disease, and neurodegenerative diseases; cardiovascular/metabolic disorders/diseases, e.g. cardiovascular risk reduction (CvRR), atherosclerosis, type I and type II diabetes and related complications (e.g. nephropathy, retinopathy), peripheral artery disease (PAD), acute heart failure and hypertension; wound healing and scar formation; inflammatory skin diseases, e.g. acne, hidradenitis suppurativa, asthma, sarcoidosis, age-related macular degeneration; cancer related diseases/disorders, e.g. myeloproliferative neoplasms, leukemias, myelodysplastic syndromes (MDS), myelofibrosis, lung cancer, colon cancer.

### Administration and Pharmaceutical Compositions

Some embodiments of the present disclosure relate to a pharmaceutical composition comprising a therapeutically effective amount of at least one compound having the structure described herein and a pharmaceutically acceptable excipient.

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein, or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, atropisomers, tautomers, polymorphs, hydrates, and solvates thereof), or pharmaceutically acceptable salts thereof; and (b) a pharmaceutically acceptable carrier, diluent, excipient or combination thereof.

In addition to the selected compound useful as described above, some embodiments include compositions containing a pharmaceutically acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, intravitreal, subcutaneous, or other parental routes of administration. In some embodiments, the compositions may be in a form suitable for subcutaneous administration. The skilled artisan will appreciate that oral and nasal compositions comprise compositions that are administered by inhalation and made using available methodologies. Depending upon the particular route of administration desired, a variety of pharmaceutically acceptable carriers well-known in the art may be used. Pharmaceutically acceptable carriers include, for example, solid or liquid fillers, diluents, hydrotropes, surface-active agents, and encapsulating substances. Optional pharmaceutically active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically acceptable carrier suitable for the preparation of unit dosage forms for peroral administration is well-known in the art. Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g., drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate.

A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose, cyclodextrins or derivatives thereof, and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7.

Antioxidant excipients may include sodium bisulfite, acetone sodium bisulfite, sodium formaldehyde sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran.

Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety.

Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt-%) basis, from about 0.01 to about 99.99 wt-% of a compound of the present technology based on the total formulation, with the balance being one or more suitable pharmaceutical excipients. Preferably, the compound is present at a level of about 1 to about 80 wt-%. Representative pharmaceutical formulations are described below.

### FORMULATION EXAMPLES

The following are representative pharmaceutical formulations containing a compound of Formula (1-I), (1-II), (1-III), (1-IV), (1-V), (1-VI), (1-VII), (1-VIII), (1-IX), (2-I), (2-II), (2-III), (2-IV), (2-V), (2-VI), or 2-VII.

### Formulation Example 1—Tablet Formulation

The following ingredients are mixed intimately and pressed into single scored tablets.

| Ingredient | Quantity per tablet, mg |
|---|---|
| compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| Ingredient | Quantity per tablet, mg |
|---|---|
| compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.0 | g |
| Veegum K (Vanderbilt Co.) | 1.0 | g |
| flavoring | 0.035 | mL |
| Colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| compounds disclosed herein | 500 mg |
| Witepsol ® H-15 | balance |

To further illustrate this invention, the following examples are included. The examples should not, of course, be construed as specifically limiting the invention. Variations of these examples within the scope of the claims are within the purview of one skilled in the art and are considered to fall within the scope of the invention as described and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

Compounds may be identified by their chemical structure and/or their chemical name. Chemical names were generated using the PerkinElmer (Waltham, MA 02451, USA) ChemDraw® Professional 19.1.1.21 nomenclature program. When the chemical structure and chemical name conflict, the chemical structure is determinative of the identity of the compound.

Compounds of Formula (1-I), (1-II), (1-III), (1-IV), (1-V), (1-VI), (1-VII), (1-VIII) and (1-IX) disclosed herein include any specific compounds within these Formulae. Specific compounds within Formulae (1-I), (1-II), (1-III), (1-IV), (1-V), (1-VI), (1-VII), (1-VIII), and (1-IX) are shown in Table 1.

### EXAMPLES

TABLE 1

| COMPOUND | STRUCTURE |
|---|---|
| 1 | 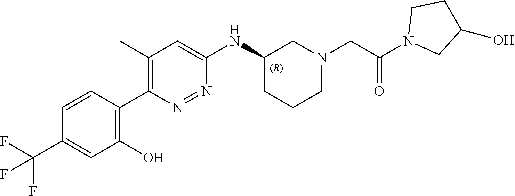 |
| 2 | 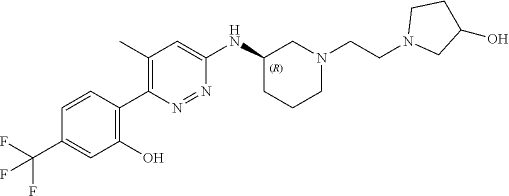 |
| 3 | 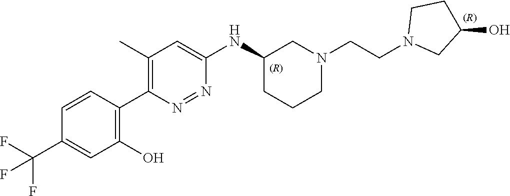 |
| 4 | 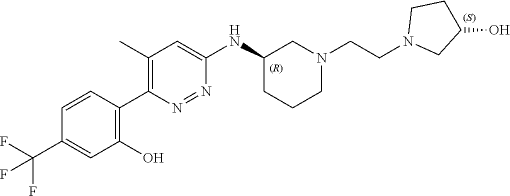 |
| 5 | 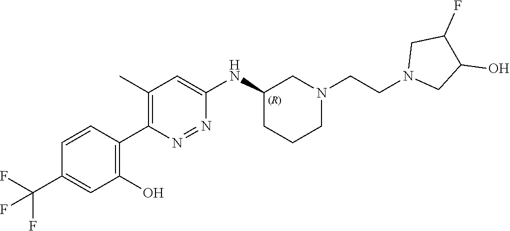 |
| 6 | 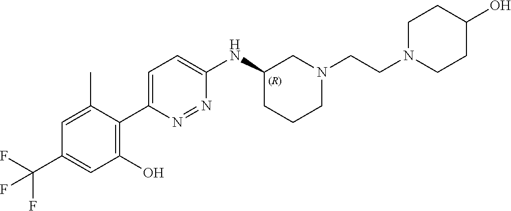 |
| 7 | 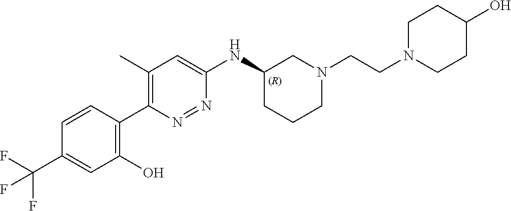 |
| 8 | 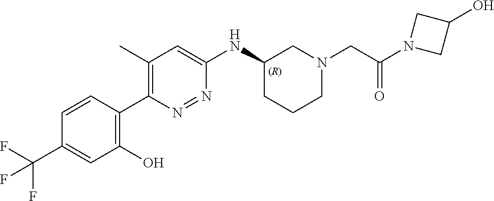 |
| 9 | 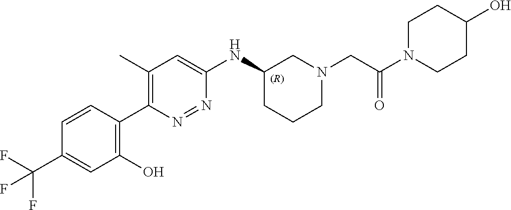 |
| 10 | 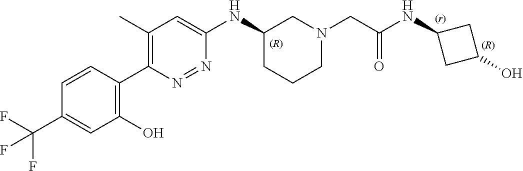 |
| 11 | 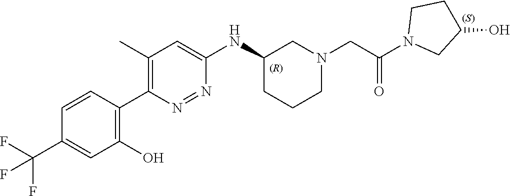 |
| 12 | 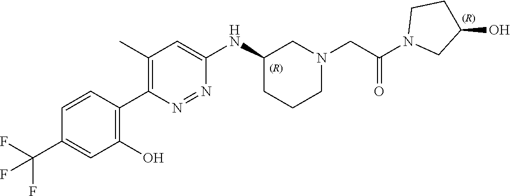 |
| 13 | 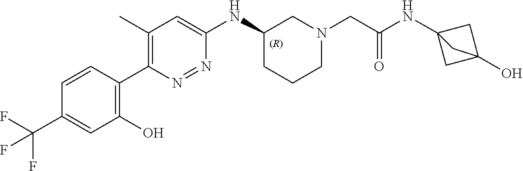 |
| 14 | 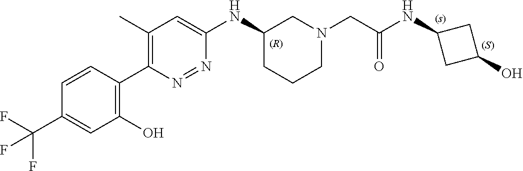 |
| 15 | 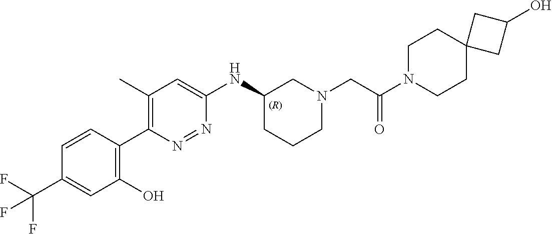 |
| 16 | 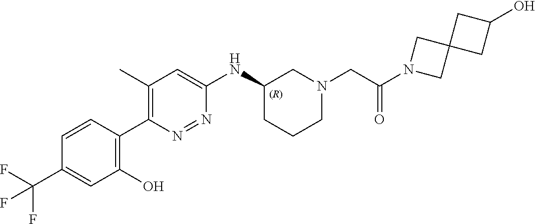 |
| 17 | 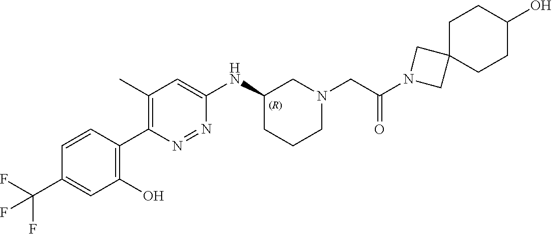 |
| 18 | 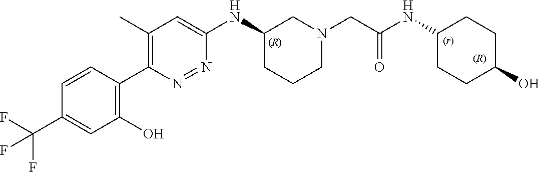 |
| 19 | 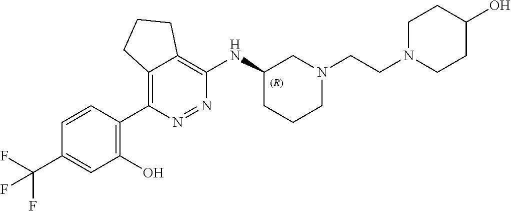 |
| 20 | 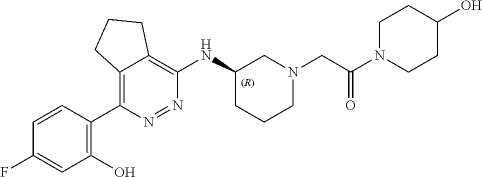 |
| 21 | 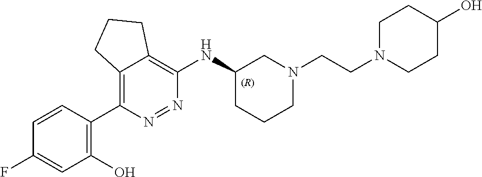 |
| 22 | 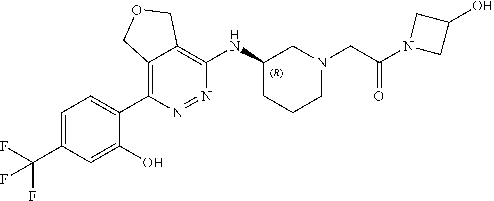 |
| 23 | 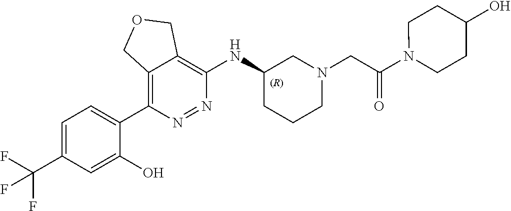 |
| 24 | 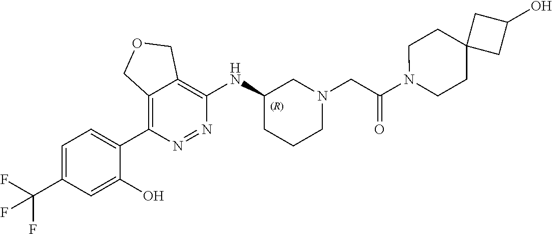 |
| 25 | 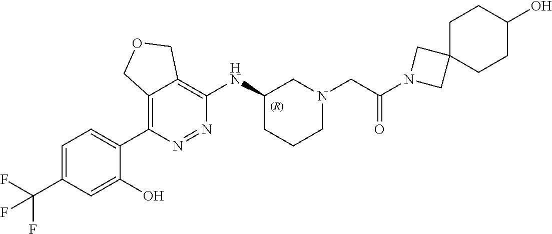 |
| 26 | 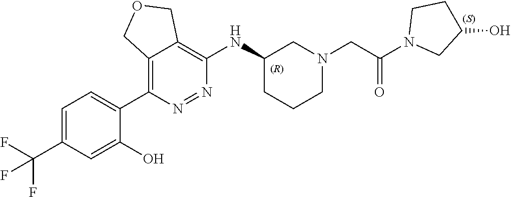 |
| 27 | 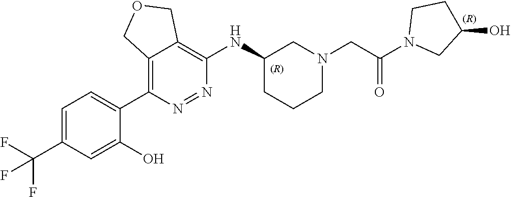 |
| 28 | 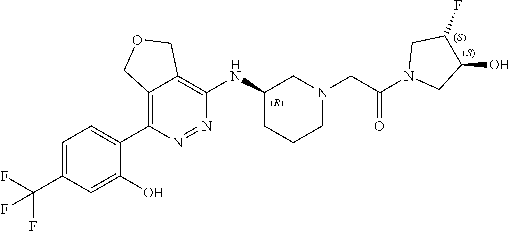 |
| 29 | 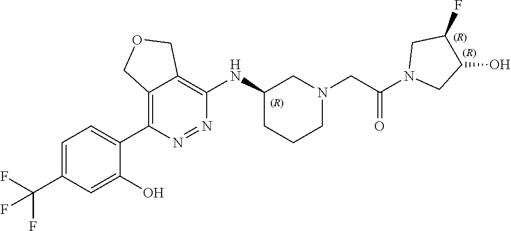 |
| 30 | 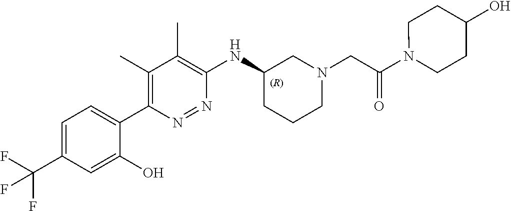 |
| 31 | 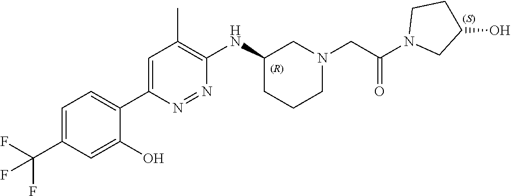 |
| 32 | 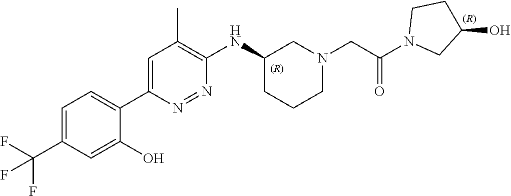 |
| 33 | 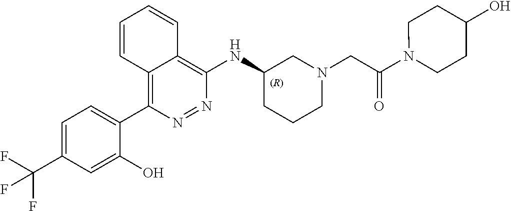 |
| 34 | 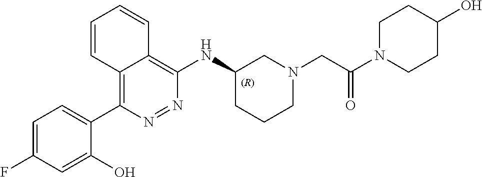 |
| 35 | 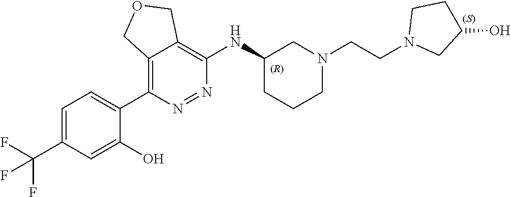 |
| 36 | 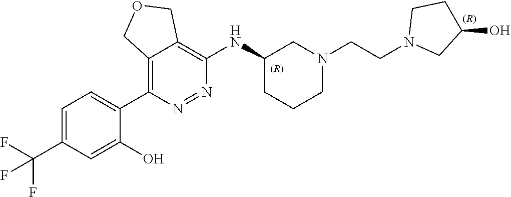 |
| 37 | 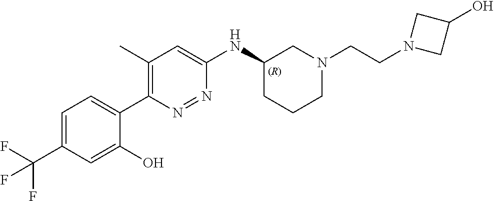 |
| 38a | 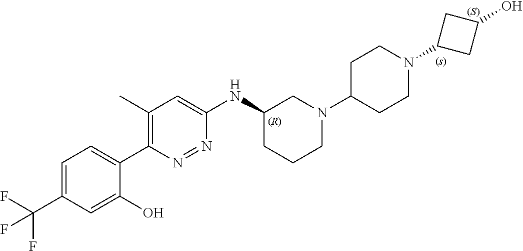 |
| 38b | 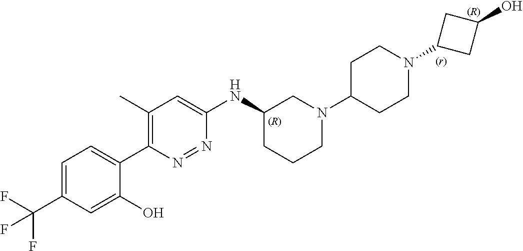 |
| 39a | 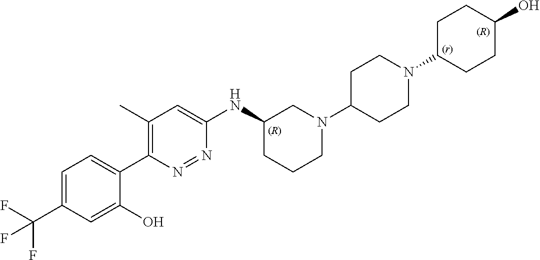 |
| 39b | 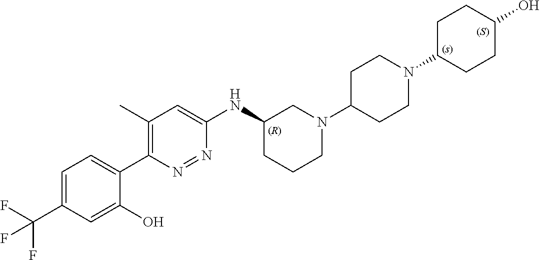 |
| 40 | 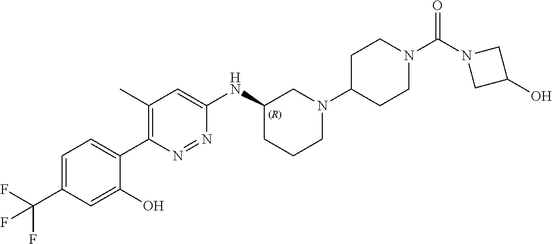 |
| 41 | 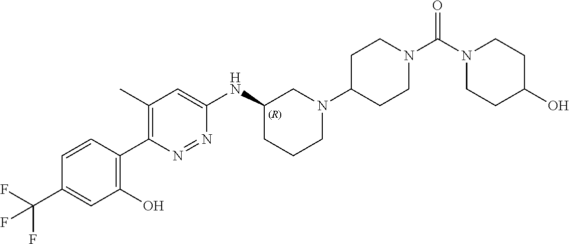 |
| 42 | 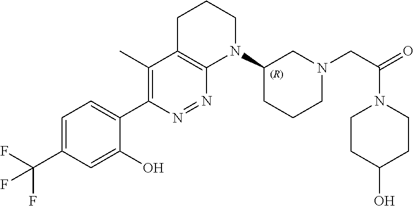 |
| 43 | 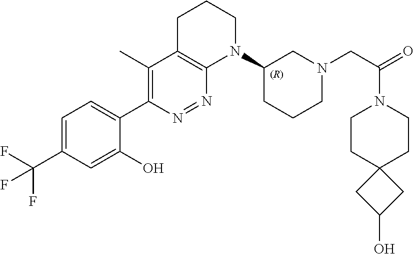 |
| 44 | 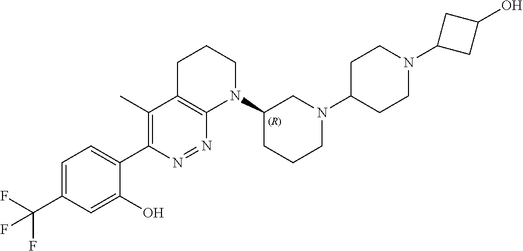 |
| 45 | 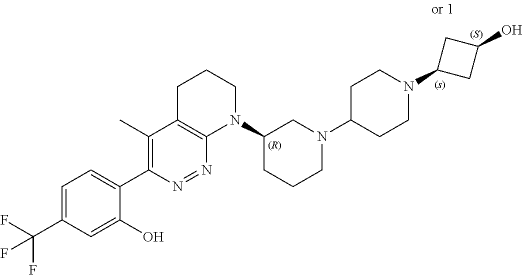 |
| 46 | 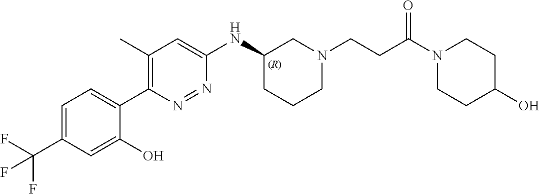 |
| 47 | 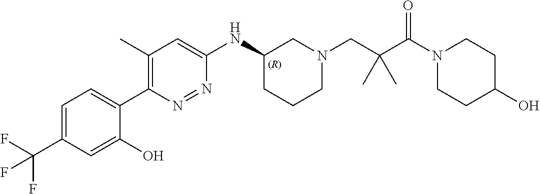 |
| 48 | 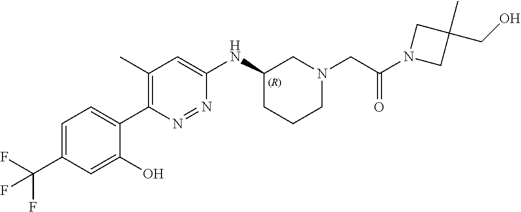 |
| 49 | 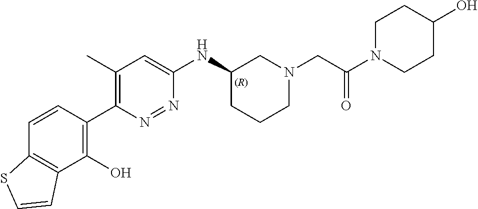 |
| 50 | 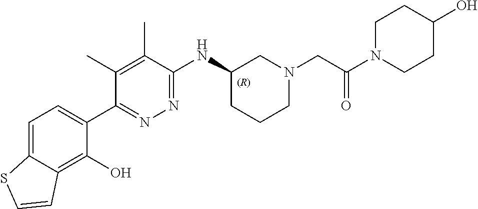 |
| 51 | 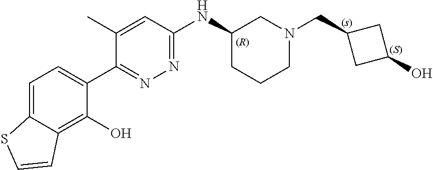 |
| 52 | 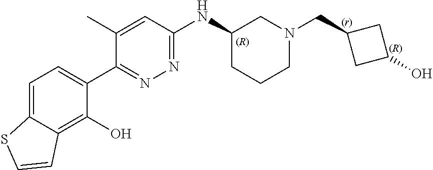 |
| 53 | 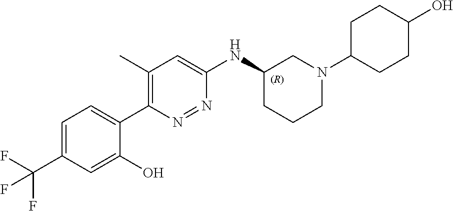 Fraction A |
| 54 | 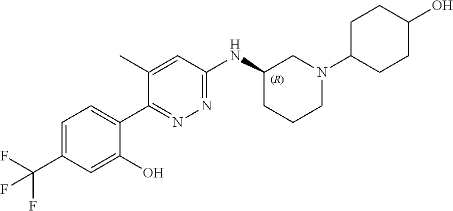 54 Fraction B |
| 55 | 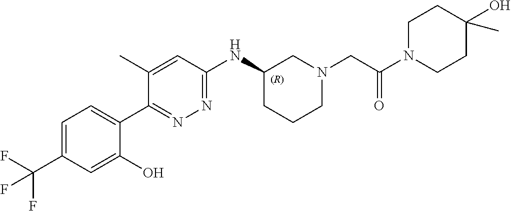 |
| 56 | 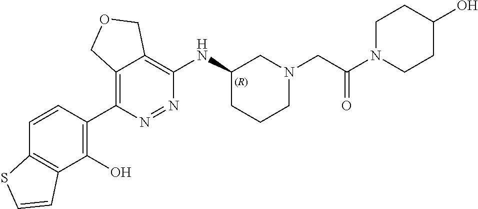 |
| 57 | 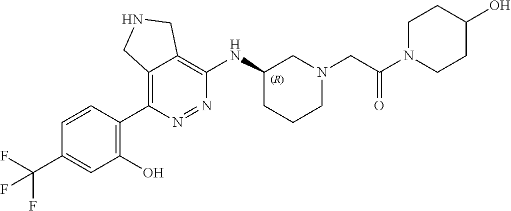 |
| 58 | 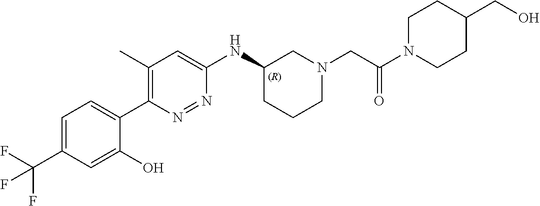 |
| 59 | 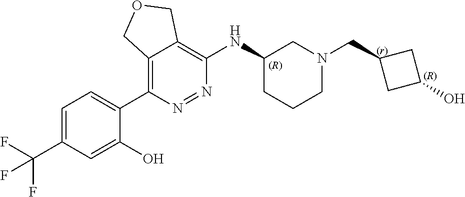 |
| 60 | 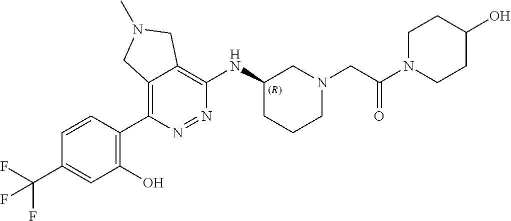 |
| 61 | 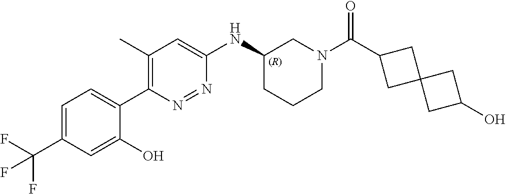 |
| 62 | 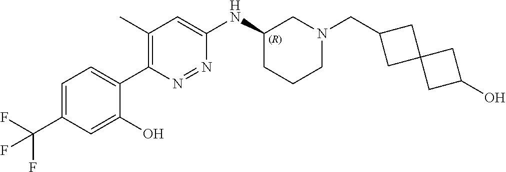 |
| 63 | 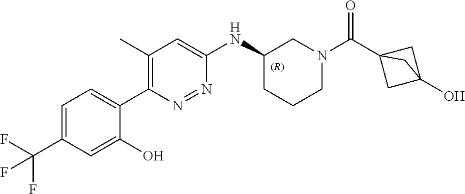 |
| 64 | 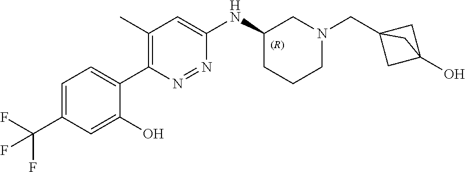 |
| 65 | 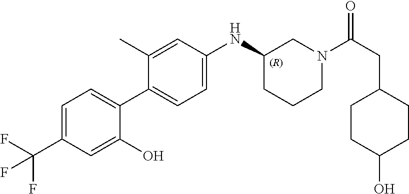 |
| 66 | 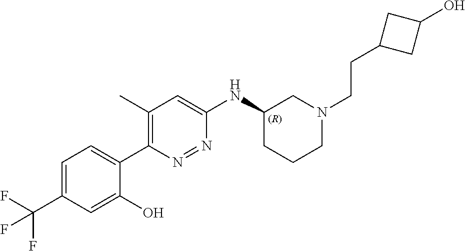 |
| 67 | 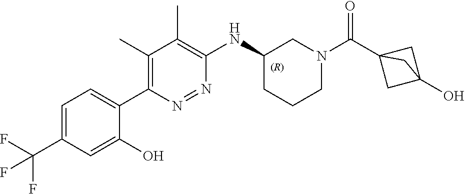 |
| 68 | 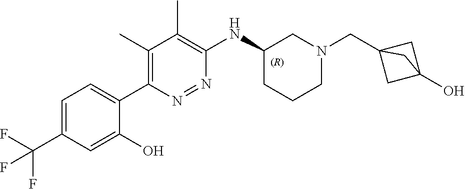 |
| 69 | 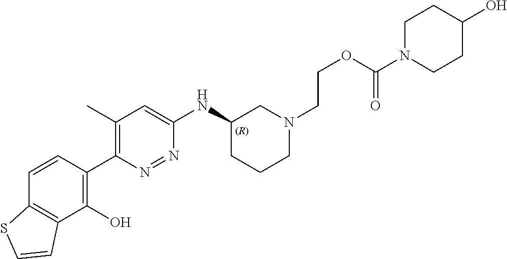 |
| 70 | 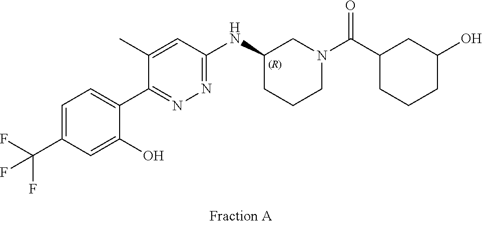 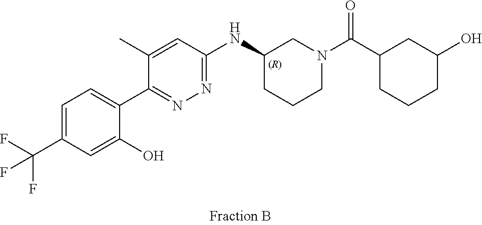 |
| 71 | 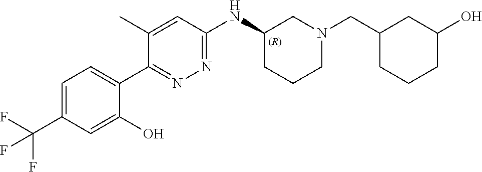 |
| 72 | 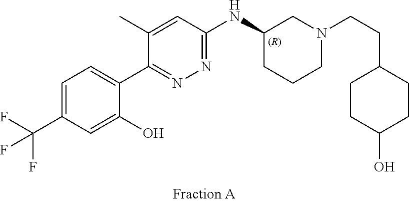 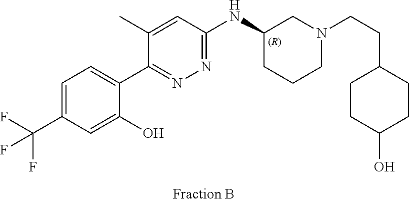 |
| 73 | 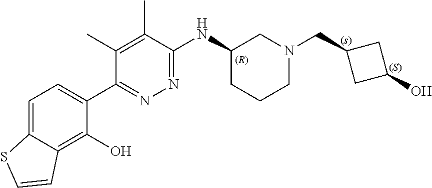 |
| 74 | 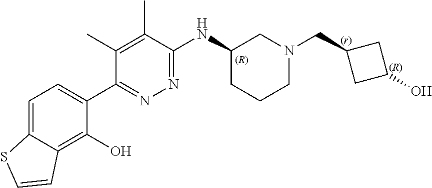 |
| 75 | 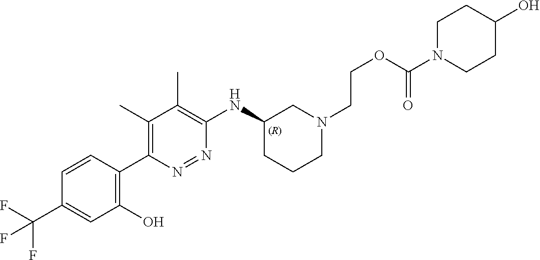 |
| 76 | 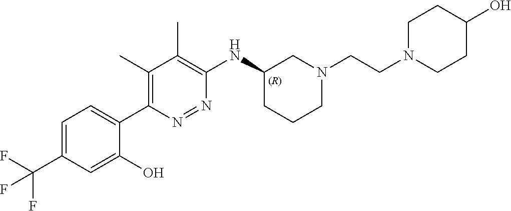 |

Compounds of Formula (2-I), (2-II), (2-III), (2-IV), (2-V), (2-VI), and (2-VII) disclosed herein include any specific compounds within these Formulae. Specific compounds within Formulae (2-I), (2-II), (2-III), (2-IV), (2-V), (2-VI), and (2-VII) are shown in Table 2.

TABLE 2

| COMPOUND | STRUCTURE |
|---|---|
| 77 | 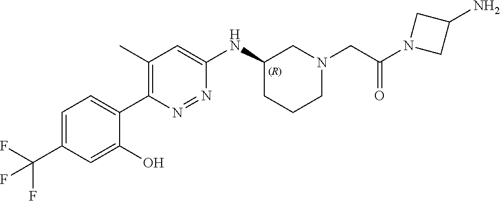 |
| 78 | 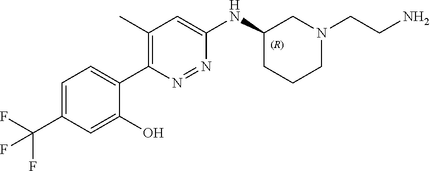 |
| 79 | 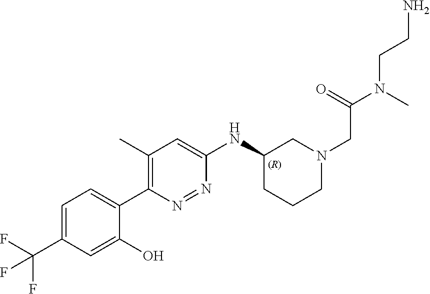 |
| 80 | 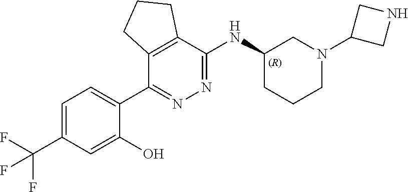 |
| 81 | 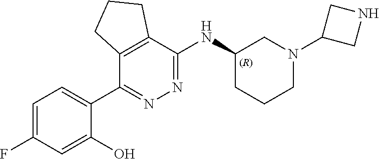 |
| 82 | 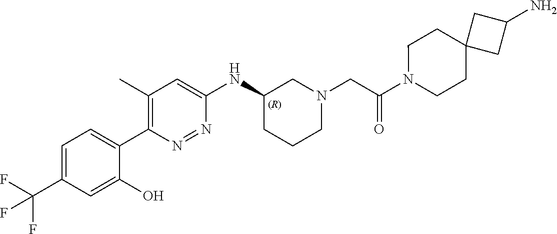 |
| 83 | 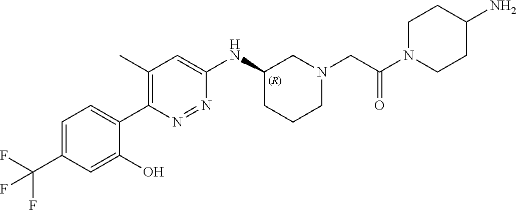 |
| 84 | 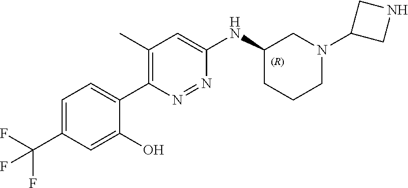 |
| 85 | 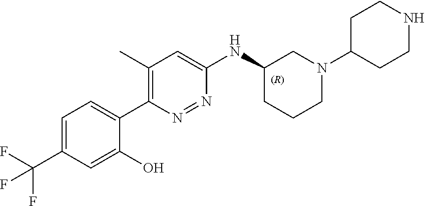 |
| 86 | 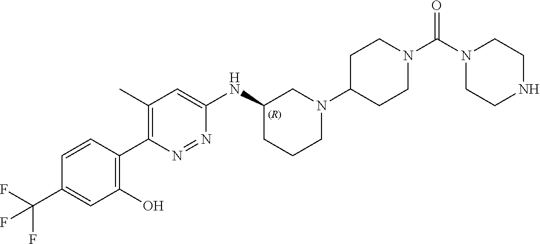 |
| 87 | 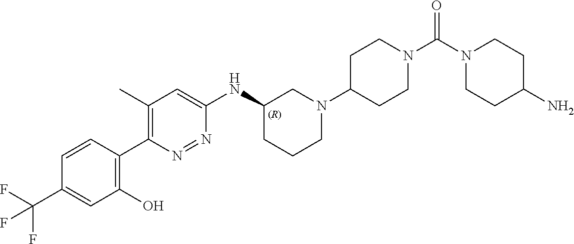 |
| 88 | 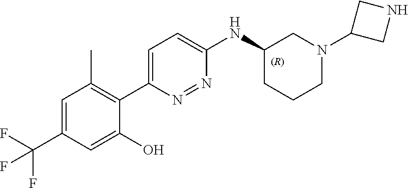 |
| 89 | 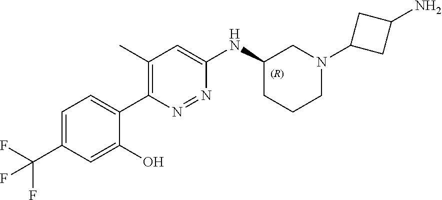 |
| 90 | 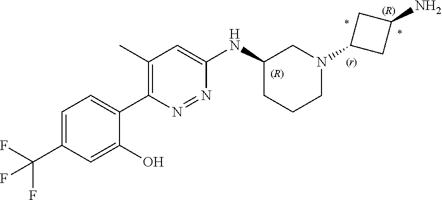 |
| 91 | 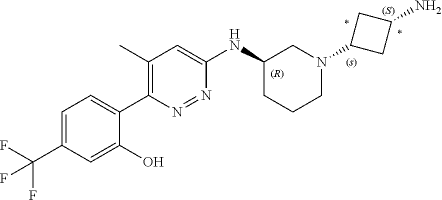 |
| 92 | 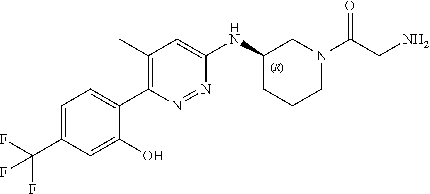 |
| 93 | 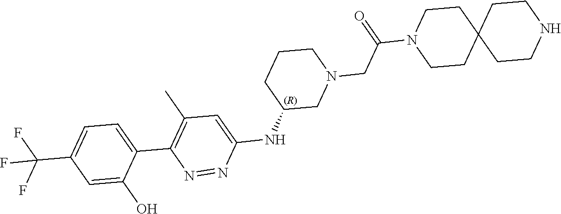 |
| 94 | 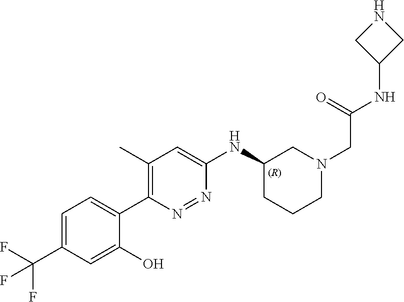 |
| 95 | 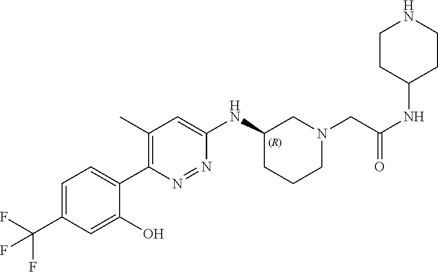 |
| 96 | 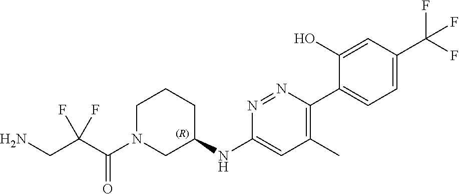 |
| 97 | 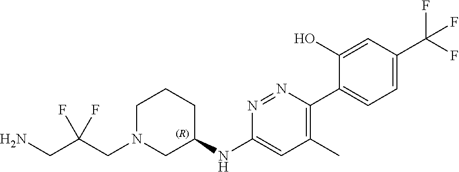 |
| 98 | 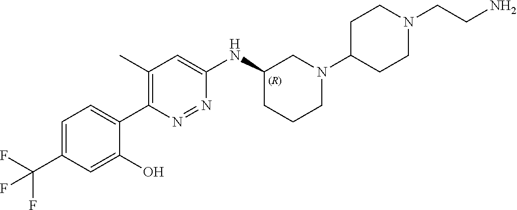 |
| 99 | 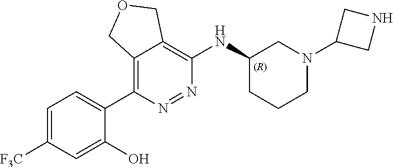 |
| 100 | 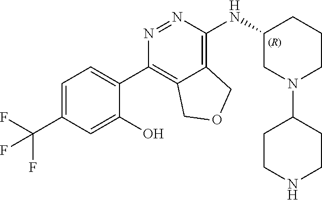 |
| 101 | 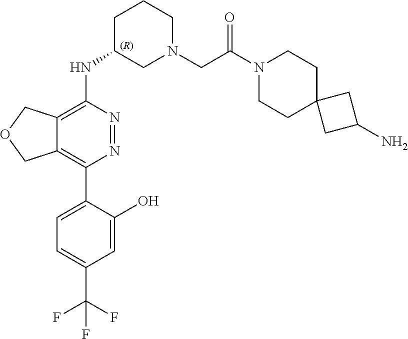 |
| 102 | 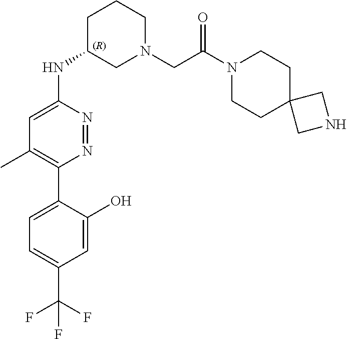 |
| 103 | 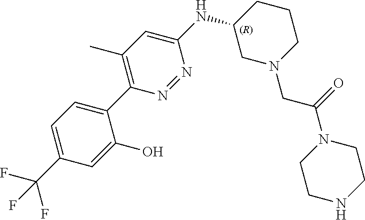 |
| 104 | 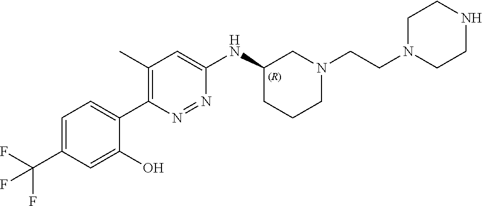 |
| 105 | 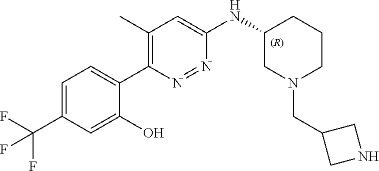 |
| 106 | 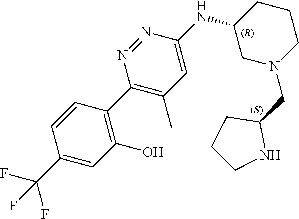 |
| 107 | 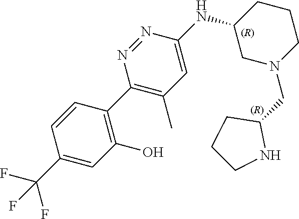 |
| 108 | 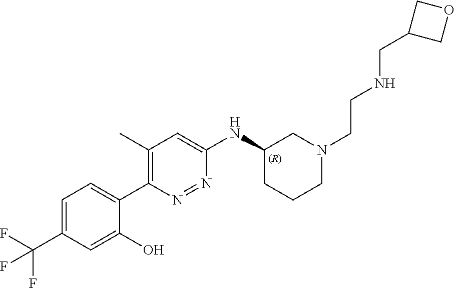 |
| 109 | 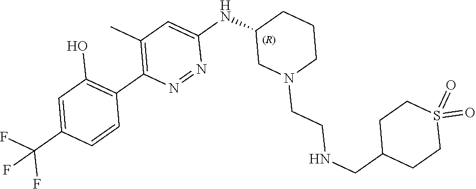 |
| 110 | 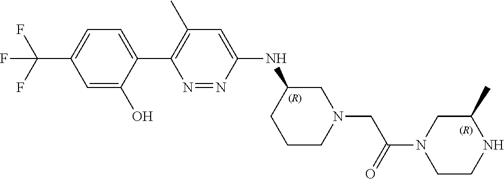 |
| 111 | 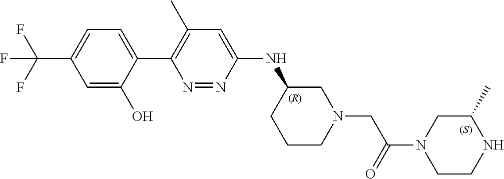 |
| 112 | 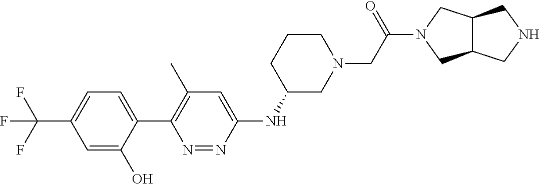 [SMILES: Cc1cc(N[C@@H]2CCCN(CC(=O)N3C[C@H]4CNC[C@H]4C3)C2)nnc1-c1ccc(C(F)(F)F)cc1O] |
| 113 | 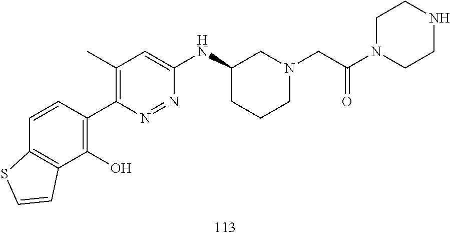 [SMILES: Cc1cc(N[C@@H]2CCCN(CC(=O)N3CCNCC3)C2)nnc1-c1ccc2sccc2c1O] |
| 114 | 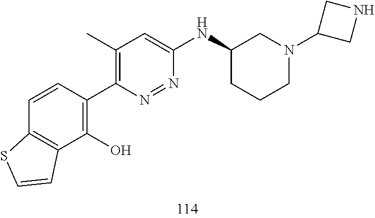 [SMILES: Cc1cc(N[C@@H]2CCCN(C3CNC3)C2)nnc1-c1ccc2sccc2c1O] |

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader and represent preferred methods for making the compounds exemplified herein. These methods are not limiting, and it will be apparent that other routes may be employed to prepare these compounds. Such methods specifically include solid phase based chemistry, including combinatorial chemistry. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
2N=a 2 normal solution of a species
ACN=acetonitrile
AIBN=azobisisobutyronitrile
aq.=aqueous
Bn=benzyl
Boc=tert-butoxycarbonyl
brine=saturated aqueous solution of sodium chloride (NaCl)
Bu=butyl
CD₃OD=deuterated methanol
CHCl₃=chloroform
CDCl₃=deuterochloroform
CH₂Cl₂=methylene chloride, or dichloromethane or DCM
Cs₂CO₃=cesium carbonate
DIEA=N,N-diisopropylethylamine
DMF=dimethylformamide
DMSO=dimethylsulfoxide
EDCI=1-ethyl-3-(3-dimethylaminopropyl) carbodiimide (EDC, EDC·HCl)
Et=ethyl
EtOAc or EA=ethyl acetate
g=gram
FA=formic acid
h=hour
¹H⁺=proton
H=hydrogen
H₂O=water
HATU=Hexafluorophosphate azabenzotriazole tetramethyl uronium
HCl=Hydrogen chloride
HOBt=hydroxybenzotriazole
HPLC=high-performance liquid chromatography
iPrOH=isopropyl alcohol
LCMS=liquid chromatographic mass spectroscopy
Me=methyl
M=molarity
m=multiplet
[M+H]⁺=molecular ion plus one proton
MeOH=methanol
min=minute
mL=milliliter
mmol=millimole
MHz=megahertz
m/z=mass to charge ratio
N₂=nitrogen
NaCl=sodium chloride
Na₂SO₄=sodium sulfate
NH₃=ammonia
NBS=N-bromo succinimide
NH₄HCO₃=ammonium hydrogen carbonate
NMR=nuclear magnetic resonance
NMP=N-methyl-2-pyrrolidone or 1-methyl-2-pyrrolidone
o/n or o.n.=overnight
PBr₃=phosphorous tribromide
PCl₅=phosphorous pentachloride
   PCC=pyridinium chlorochromate
PEG=polyethylene glycol
Ph=phenyl
pH=negative logarithm of hydrogen ion concentration
PPh₃Cl₂=triphenylphosphine dichloride
ppm=parts per million
q=quartet
rt=room temperature
s=singlet
sat.=saturated
t=triplet
SFC=supercritical fluid chromatography
TBSCl=tert-butyldimethylsilyl chloride
tBu=tert-butyl
TFA=trifluoroacetic acid
THF=tetrahydrofuran
TLC=thin layer chromatography
TMS=trimethylsilyl

### EXAMPLES

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1: 2-((R)-3-((6-(2-Hydroxy-4-(trifluoromethyl)phenyl)-5-methylpyridazin-3-yl)amino)piperidin-1-yl)-1-(3-hydroxypyrrolidin-1-yl)ethan-1-one (1)

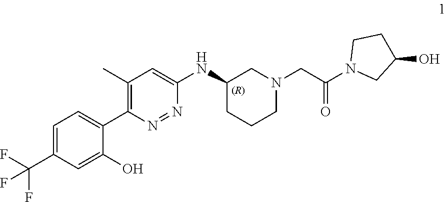

### Synthetic Scheme:

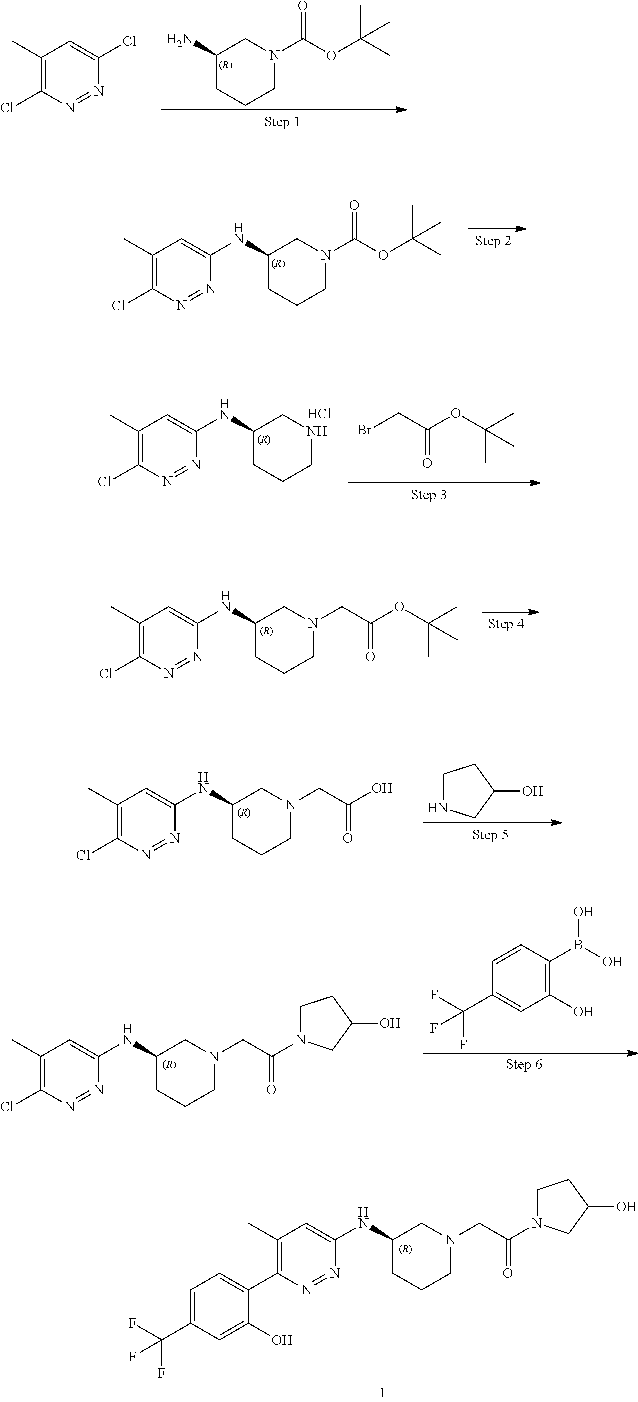

### Step 1: Synthesis of tert-butyl (3R)-3-[(6-chloro-5-methylpyridazin-3-yl)amino]piperidine-1-carboxylate

A brown solution of 3,6-dichloro-4-methylpyridazine (5.00 g, 30.7 mmol) and tert-butyl (3R)-3-aminopiperidine-1-carboxylate (6.76 g, 33.7 mmol) and DIEA (16.0 mL, 11.89 g, 92.0 mmol) in NMP (25 mL) was stirred overnight at 150° C. (oil bath). To this solution were added EtOAc (100 mL) and water (100 mL). After separation of the phases, the aqueous phase was extracted with EtOAc (2×50 mL). The combined organic phases were washed with brine (2×200 mL), dried over Na₂SO₄, filtered, and the filtrate was concentrated under reduced pressure using a rotary evaporator. After concentration, the suspension was subjected to silica gel column chromatography purification eluting with PE/EtOAc gradients (0~50%) to afford tert-butyl (3R)-3-[(6-chloro-5-methylpyridazin-3-yl)amino]piperidine-1-carboxylate as a yellow oil (1.3 g, 13.0% yield). LC/MS: mass calcd for C₁₅H₂₃ClN₄O₂: 326.15, found: m/z=327.15 [M+H]⁺.

### Step 2: Synthesis of 6-chloro-5-methyl-N-[(3R)-piperidin-3-yl]pyridazin-3-amine hydrochloride

To a solution of tert-butyl (3R)-3-[(6-chloro-5-methylpyridazin-3-yl)amino]piperidine-1-carboxylate (680 mg, 2.08 mmol) in DCM (3 mL) was added hydrogen chloride (HCl) (3 mL, 12.0 mmol, 4 M in 1,4-dioxane) at 0° C. (ice bath). The resulting solution was stirred at rt for 2 h. The reaction mixture was concentrated under reduced pressure using a rotary evaporator to afford 6-chloro-5-methyl-N-[(3R)-piperidin-3-yl]pyridazin-3-amine hydrochloride as a colorless solid (550 mg, 90.4% yield). LC/MS: mass calcd for C₁₀H₁₅ClN₄: 226.10, found: m/z=227.00 [M+H]⁺.

### Step 3: Synthesis of tert-butyl (R)-2-(3-((6-chloro-5-methylpyridazin-3-yl)amino)piperidin-1-yl)acetate

A brown solution of 6-chloro-5-methyl-N-[(3R)-piperidin-3-yl]pyridazin-3-amine (600 mg, 2.65 mmol), tert-butyl 2-bromoacetate (516 mg, 2.65 mmol, 1.0 eq.) and DIEA (1.84 mL, 1.37 gg, 10.59 mmol) in ACN (7 mL) was stirred at 60° C. for 1 h. The residue was subjected to silica gel column purification eluting with PE/EtOAc gradients (0~100%) to afford tert-butyl 2-[(3R)-3-[(6-chloro-5-methylpyridazin-3-yl)amino]piperidin-1-yl]acetate as a yellow solid (650 mg, 68.7% yield). LC/MS: mass calcd for C₁₆H₂₅ClN₄O₂: 340.17, found: m/z=341.10 [M+H]⁺.

### Step 4: Synthesis of (R)-2-(3-((6-chloro-5-methylpyridazin-3-yl)amino)piperidin-1-yl)acetic Acid

A light yellow suspension of tert-butyl 2-[(3R)-3-[(6-chloro-5-methylpyridazin-3-yl)amino]piperidin-1-yl]acetate (600 mg, 1.76 mmol) in DCM (4 mL) and a HCl (4 mL, 16 mmol, 4 M HCl in 1,4-dioxane) was stirred at 40° C. overnight. The solvent was removed under reduced pressure using a rotary evaporator to afford [(3R)-3-[(6-chloro-5-methylpyridazin-3-yl)amino]piperidin-1-yl]acetic acid as a light yellow solid (500 mg, 90.3% yield). LC/MS: mass calcd for C₁₂H₁₇ClN₄O₂: 284.10, found: 285.00 m/z=[M+H]⁺.

### Step 5: Synthesis of 2-((R)-3-((6-chloro-5-methylpyridazin-3-yl)amino)piperidin-1-yl)-1-(3-hydroxypyrrolidin-1-yl)ethan-1-one

A yellow solution of [(3R)-3-[(6-chloro-5-methylpyridazin-3-yl)amino]piperidin-1-yl]acetic acid (250 mg, 0.88 mmol), pyrrolidin-3-ol (92 mg, 1.05 mmol, 1.2 eq.), HATU (501 mg, 1.32 mmol, 1.5 eq.) and DIEA (764 μL, 567 mg, 4.39 mmol, 5.0 eq.) in DMF (2 mL) was stirred at rt for 2 h. After evaporation of the volatiles under reduced pressure in high vacuum, the crude reaction mixture was subjected to reverse phase column chromatography (10~50% ACN/water (0.05% NH₄HCO₃)) to afford 2-[(3R)-3-[(6-chloro-5-methylpyridazin-3-yl)amino]piperidin-1-yl]-1-(3-hydroxypyrrolidin-1-yl)ethanone as a light yellow oil (200 mg, 57.9% yield). LC/MS: mass calcd for C₁₆H₂₄ClN₅O₂: 353.16, found: m/z=354.20 [M+H]⁺.

### Step 6: Synthesis of 2-((R)-3-((6-(2-hydroxy-4-(trifluoromethyl)phenyl)-5-methylpyridazin-3-yl)amino)piperidin-1-yl)-1-(3-hydroxypyrrolidin-1-yl)ethan-1-one (1)

A brown suspension of 2-[(3R)-3-[(6-chloro-5-methylpyridazin-3-yl)amino]piperidin-1-yl]-1-(3-hydroxypyrrolidin-1-yl)ethanone (200 mg, 0.57 mmol, 1.0 eq.), 2-hydroxy-4-(trifluoromethyl)phenylboronic acid (175 mg, 0.85 mmol, 1.5 eq.), Pd(PPh₃)₄ (20 mg, 0.017 mmol, 0.03 eq.), NaHCO₃ (142 mg, 1.70 mmol, 3.0 eq.) in dioxane (3 mL) and water (1.5 mL) was stirred in a sealed tube at 150° C. under an atmosphere of N₂ for 1 h. After the reaction mixture was cooled down to rt, the solution was subjected to silica gel column purification eluting with DCM/MeOH gradients (0~10%) to afford a light brown oil (150 mg) of the pre-purified reaction product. The material was further purified by preparative RP-HPLC using the following conditions Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 26% B to 46% B in 7 min, 46% B; Wavelength: 254 nm; RT1: 5.73 min. After lyophilization of the combined product containing fractions, the target compound 2-[(3R)-3-({6-[2-hydroxy-4-(trifluoromethyl)phenyl]-5-methylpyridazin-3-yl}amino)piperidin-1-yl]-1-(3-hydroxypyrrolidin-1-yl) ethenone (1) was obtained as a light yellow solid (36.0 mg, 13.2% yield). LC/MS: mass calcd for C₂₃H₂₈F₃N₅O₃: 479.21, found: m/z=480.15 [M+H]⁺. ¹H NMR (300 MHz, DMSO-d₆): δ 10.51 (s, 1H), 7.43-7.34 (m, 1H), 7.26-7.16 (m, 2H), 6.73-6.58 (m, 2H), 5.09-4.81 (m, 1H), 4.35-4.17 (m, 1H), 4.02 (s, 1H), 3.54-3.47 (m, 2H), 3.23-3.14 (m, 2H), 3.23-3.04 (m, 2H), 3.00-2.85 (m, 1H), 2.77-2.58 (m, 1H), 2.31-2.07 (m, 2H), 2.00 (s, 3H), 1.94-1.63 (m, 4H), 1.61-1.44 (m, 1H), 1.39-1.23 (m, 1H) ppm. ¹⁹F NMR (282 MHz, DMSO-d₆): δ −61.17 ppm.

### Example 2: 1-(2-((R)-3-((6-(2-Hydroxy-4-(trifluoromethyl)phenyl)-5-methylpyridazin-3-yl)amino)piperidin-1-yl)ethyl)pyrrolidin-3-ol (2)

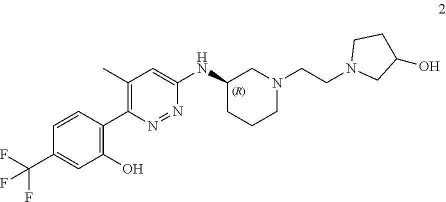

### Synthetic Scheme:

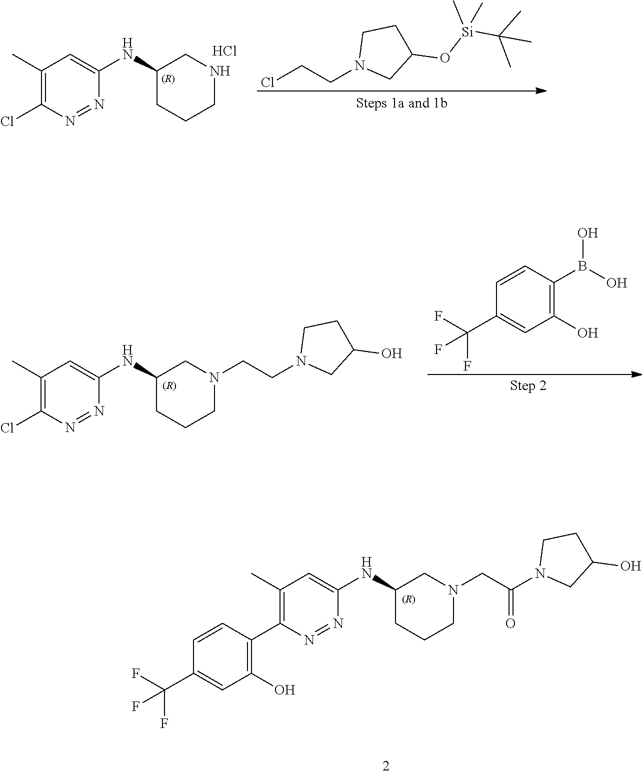

### Steps 1a and 1b: Synthesis of 1-(2-((R)-3-((6-chloro-5-methylpyridazin-3-yl)amino)piperidin-1-yl)ethyl)pyrrolidin-3-ol

A brown suspension of 6-chloro-5-methyl-N-[(3I)-piperidin-3-yl]pyridazin-3-amine hydrochloride (200 mg, 0.76 mmol), 3-[(tert-butyldimethylsilyl)oxy]-1-(2-chloroethyl)pyrrolidine (221 mg, 0.84 mmol, 1.1 eq.) (rac-3-[(tert-butyldimethylsilyl)oxy]-1-(2-chloroethyl)pyrrolidine was prepared in analogy to (3S)-3-[(tert-butyldimethylsilyl)oxy]-1-(2-chloroethyl)pyrrolidine described in Example 4 but using rac-pyrrolidin-3-ol hydrochloride as a starting material), NaI (114 mg, 0.76 mmol, 1.0 eq.) and DIEA (398 μL, 295 mg, 2.28 mmol, 3.0 eq.) in ACN (3 mL) was stirred at 80° C. for 2 h. After evaporation of the volatiles under reduced pressure using a rotary evaporator, the suspension was submitted to reversed phase column purification (5~40% ACN/water+0.05 vol-% TFA) to afford a brown oil which was dissolved in 2 mL of a DCM-TFA mixture (1:1, v/v)) and stirred at rt for 1 h. After removal of solvents at 70° C., the residue was subjected to silica gel column purification eluting with DCM/MeOH gradients (0~10%) to afford 1-{2-[(3R)-3-[(6-chloro-5-methylpyridazin-3-yl)amino]piperidin-1-yl]ethyl}pyrrolidin-3-ol as a brown oil (150 mg, 58.1% yield). LC/MS: mass calcd for C₁₆H₂₆ClN₅O: 339.18, found: m/z=340.10 [M+H]⁺.

### 2: Step Synthesis of 1-(2-((R)-3-((6-(2-hydroxy-4-(trifluoromethyl)phenyl)-5-methylpyridazin-3-yl)amino)piperidin-1-yl)ethyl)pyrrolidin-3-ol (2)

A brown suspension of 1-{2-[(3R)-3-[(6-chloro-5-methylpyridazin-3-yl)amino]piperidin-1-yl]ethyl}pyrrolidin-3-ol (150 mg, 0.44 mmol, 1.0 eq.), 2-hydroxy-4-(trifluoromethyl)phenylboronic acid (145 mg, 0.71 mmol, 1.6 eq.), Pd(PPh₃)₄ (15 mg, 0.013 mmol, 0.03 eq.) and NaHCO₃ (111 mg, 1.32 mmol, 3.0 eq.) in dioxane (2 mL) and H₂O (1 mL) was stirred in a sealed tube at 150° C. for 1 h under an atmosphere of N₂. After the reaction mixture cooled to rt, the solvents were removed under reduced pressure using a rotary evaporator, and the residue was subjected to silica gel column purification eluting with DCM/MeOH gradients (0~10%) to afford a brown solid (100 mg). The solid was further purified by preparative RP-HPLC under the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 22% B to 44% B in 7 min, 44% B; Wavelength: 254 nm; RT1: 6.07 min. After lyophilization of the combined product containing fractions, the target material 1-{2-[(3R)-3-({6-[2-hydroxy-4-(trifluoromethyl)phenyl]-5-methylpyridazin-3-yl}amino)piperidin-1-yl]ethyl}pyrrolidin-3-ol (2) was obtained as a colorless solid (20.3 mg, 9.6% yield). LC/MS: mass calcd for C₂₃H₃₀F₃N₅O₂: 465.24, found: m/z=466.20 [M+H⁺]. ¹H NMR (400 MHz, Methanol-d₄): δ 7.42-7.40 (m, 1H), 7.24-7.18 (m, 2H), 6.80 (s, 1H), 4.40-4.38 (m, 1H), 4.14-4.10 (m, 1H), 3.12-3.06 (m, 1H), 2.98-2.67 (m, 9H), 2.35-2.28 (m, 1H), 2.21-2.08 (m, 5H), 1.99-1.91 (m, 1H), 1.89-1.66 (m, 3H), 1.55-1.46 (m, 1H) ppm. ¹⁹F NMR (376 MHz, Methanol-d₄): δ −64.26 ppm.

### Example 3: (R)-1-(2-((R)-3-((6-(2-Hydroxy-4-(trifluoromethyl)phenyl)-5-methylpyridazin-3-yl)amino)piperidin-1-yl)ethyl)pyrrolidin-3-ol (3)

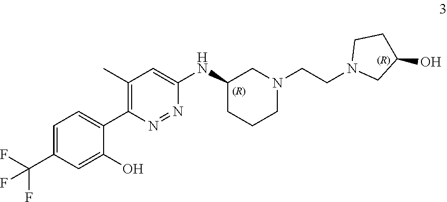

### Synthetic Scheme:

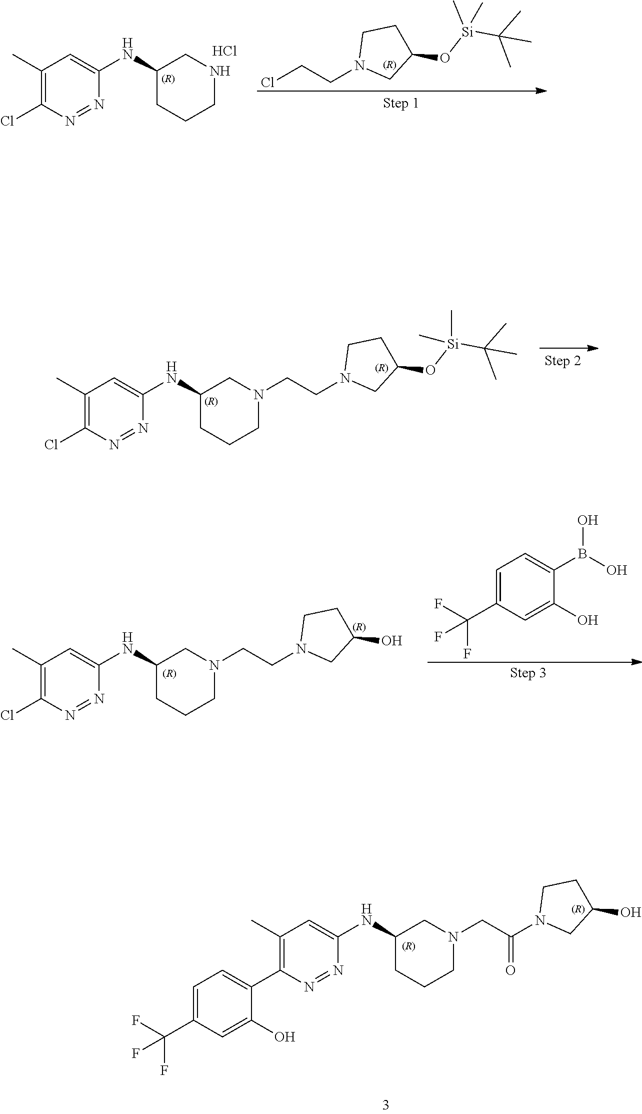

### Step 1: Synthesis of N—((R)-1-(2-((R)-3-((tert-butyldimethylsilyl)oxy)pyrrolidin-1-yl)ethyl)piperidin-3-yl)-6-chloro-5-methylpyridazin-3-amine

A brown suspension of (3R)-3-[(tert-butyldimethylsilyl)oxy]-1-(2-chloroethyl)pyrrolidine (303 mg, 1.15 mmol, 1.4 eq.) ((3R)-3-[(tert-butyldimethylsilyl)oxy]-1-(2-chloroethyl)pyrrolidine was prepared in analogy to (3S)-3-[(tert-butyldimethylsilyl)oxy]-1-(2-chloroethyl)pyrrolidine described in Example 4 but using (3S)-pyrrolidin-3-ol hydrochloride as a starting material), 6-chloro-5-methyl-N-[(3R)-piperidin-3-yl]pyridazin-3-amine (200 mg, 0.88 mmol, 1.0 eq.), NaI (132 mg, 0.88 mmol, 1.0 eq.) and DIEA (461 μL, 342 mg, 2.65 mmol, 3.0 eq.) in ACN (5 mL) was stirred at 80° C. for 2 h. After the reaction mixture cooled to rt, DCM (20 mL) and water (10 mL) were added to the suspension. After separation of the phases, the aqueous phase was extracted with DCM (2×10 mL). The combined organic phases were washed with brine (2×50 mL), dried over Na₂SO₄, filtered and the filtrate was concentrated under reduced pressure using a rotary evaporator. The residue was subjected to silica gel column purification eluting with DCM/MeOH gradients (0~5%) to afford N-[(3R)-1-{2-[(3R)-3-[(tert-butyldimethylsilyl)oxy]pyrrolidin-1-yl]ethyl}piperidin-3-yl]-6-chloro-5-methylpyridazin-3-amine as a colorless oil (180 mg, 44.9% yield). LC/MS: mass calcd for C₂₂H₄₀ClN₅OSi: 453.27, found: m/z=454.35 [M+H]⁺.

### Step 2: Synthesis of (R)-1-(2-((R)-3-((6-chloro-5-methylpyridazin-3-yl)amino)piperidin-1-yl)ethyl)pyrrolidin-3-ol

A colorless solution of N-[(3R)-1-{2-[(3R)-3-[(tert-butyldimethylsilyl)oxy]pyrrolidin-1-yl]ethyl}piperidin-3-yl]-6-chloro-5-methylpyridazin-3-amine (180 mg, 0.40 mmol) in DCM (2 mL) and HCl (2 mL, 4 M in 1,4-dioxane, 8.0 mmol, 20 eq.) was stirred at rt for 3 h. After concentration under reduced pressure using a rotary evaporator, (3R)-1-{2-[(3R)-3-[(6-chloro-5-methylpyridazin-3-yl)amino]piperidin-1-yl]ethyl}pyrrolidin-3-ol was afforded as a colorless solid (120 mg, 89.1% yield) which was used without further isolation and characterization. LC/MS: mass calcd for C₁₆H₂₆ClN₅O: 339.18, found: m/z=340.20 [M+H]⁺.

### Step 3: Synthesis of (R)-1-(2-((R)-3-((6-(2-hydroxy-4-(trifluoromethyl)phenyl)-5-methylpyridazin-3-yl)amino)piperidin-1-yl)ethyl)pyrrolidin-3-ol (3)

A red suspension of (3R)-1-{2-[(3R)-3-[(6-chloro-5-methylpyridazin-3-yl)amino]piperidin-1-yl]ethyl}pyrrolidin-3-ol (120 mg, 0.35 mmol), 2-hydroxy-4-(trifluoromethyl)phenylboronic acid (109 mg, 0.53 mmol, 1.5 eq.), NaHCO₃ (60 mg, 0.71 mmol, 2.0) and Pd(PPh₃)₄ (13 mg, 0.011 mmol, 0.03 eq) in 1,4-dioxane (1.5 mL) and H₂O (3 mL) was stirred in a sealed tube at 150° C. for 1 h under an atmosphere of N₂. After concentration under reduced pressure using a rotary evaporator, the residue was subjected to silica gel column chromatography eluting with DCM/MeOH gradients (0~5%) to afford a white solid, which was subjected to preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min, 57% B; Wavelength: 254 nm; RT1: 6 min. After lyophilization of the combined fractions, the target material (3R)-1-{2-[(3R)-3-({6-[2-hydroxy-4-(trifluoromethyl)phenyl]-5-methylpyridazin-3-yl}amino)piperidin-1-yl]ethyl}pyrrolidin-3-ol (3) was obtained as a colorless solid (22.4 mg, 13.6% yield). LC/MS: mass calcd for C₂₃H₃₀F₃N₅O₂: 465.24, found: m/z=466.20 [M+H]⁺. ¹H NMR (400 MHz, Methanol-d₄): δ 7.44-7.37 (m, 1H), 7.26-7.14 (m, 2H), 6.79 (s, 1H), 4.43-4.33 (m, 1H), 4.20-4.08 (m, 1H), 3.16-3.03 (m, 1H), 2.97-2.80 (m, 2H), 2.79-2.66 (m, 4H), 2.65-2.51 (m, 3H), 2.39-2.26 (m, 1H), 2.25-2.06 (m, 5H), 2.03-1.91 (m, 1H), 1.90-1.61 (m, 3H), 1.59-1.41 (m, 1H). ¹⁹F NMR (376 MHz, Methanol-d₄): δ −64.26 ppm.

### Example 4: (S)-1-(2-((R)-3-((6-(2-Hydroxy-4-(trifluoromethyl)phenyl)-5-methylpyridazin-3-yl)amino)piperidin-1-yl)ethyl)pyrrolidin-3-ol (4)

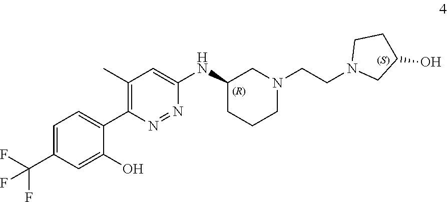

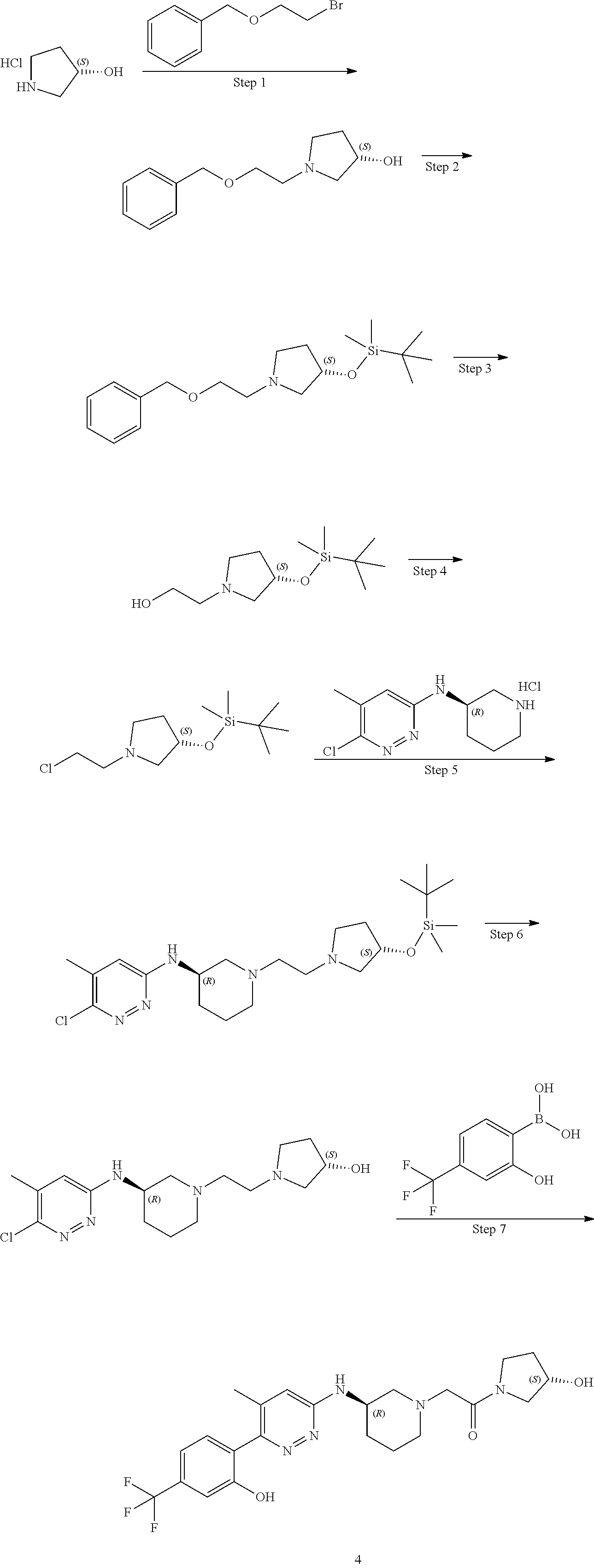

### Step 1: Synthesis of (S)-1-(2-(benzyloxy)ethyl)pyrrolidin-3-ol

To a solution of [(2-bromoethoxy)methyl]benzene (2.00 g, 9.30 mmol, 1.0 eq.) and (3S)-pyrrolidin-3-ol hydrochloride (1.26 g, 10.23 mmol, 1.1 eq.) in ACN (30 mL) was added solid K₂CO₃ (3.86 g, 27.9 mmol, 3.0 eq.) in a sealed tube. The reaction mixture was stirred at 80° C. for 2 h. The reaction mixture was diluted with a saturated aqueous NH₄Cl solution (50 mL) and the mixture was extracted with DCM (3×100 mL). The combined organic phases were dried over Na₂SO₄, filtered, and the filtrate was concentrated under reduced pressure using a rotary evaporator to afford (3S)-1-[2-(benzyloxy)ethyl]pyrrolidin-3-ol as a yellow oil (1.00 g, 41.3% yield) which was used without further isolation and characterization. LC/MS: mass calcd, for C₁₃H₁₉NO₂: 221.14, found: m/z=222.15 [M+H]⁺.

### Step 2: Synthesis of (S)-1-(2-(benzyloxy)ethyl)-3-((tert-butyldimethylsilyl)oxy)pyrrolidine

To a solution of (3S)-1-[2-(benzyloxy)ethyl]pyrrolidin-3-ol (1.00 g, 4.52 mmol, 1.0 eq.) in DCM (10 mL) were added solid TBSCl (818 mg, 5.42 mmol, 1.2 eq.) and 1H-imidazole (616 mg, 9.04 mmol, 2.0 eq.). The reaction mixture was stirred at rt for overnight. The reaction mixture was concentrated under reduced pressure using a rotary evaporator, and the residue was subjected to silica gel column purification eluting with PE/Et₂O (0~100%) gradients to afford (3S)-1-[2-(benzyloxy)ethyl]-3-[(tert-butyldimethylsilyl)oxy]pyrrolidine after concentration of the product containing fractions and evaporation of the solvent under reduced pressure using a rotary evaporator as a yellow oil (1.4 g, 83.1% yield). LC/MS: mass calcd, for C₁₉H₃₃NO₂Si: 335.23, found: 336.15 m/z=[M+H]⁺.

### Step 3: Synthesis of (S)-2-(3-((tert-butyldimethylsilyl)oxy)pyrrolidin-1-yl)ethan-1-ol

To a solution of (3S)-1-[2-(benzyloxy)ethyl]-3-[(tert-butyldimethylsilyl)oxy]pyrrolidine (1.4 g, 4.17 mmol) in 30 mL of a mixture of EA/t-BuOH (1:1,v/v) was added 10 wt-% Pd/C (1.4 g, 100 wt-%). The resulting suspension was stirred at 50° C. for overnight under an atmosphere of H₂ at atmospheric pressure. After filtration over silica gel, the solution was concentrated under reduced pressure using a rotary evaporator to afford 2-[(3S)-3-[(tert-butyldimethylsilyl)oxy]pyrrolidin-1-yl]ethanol as a yellow oil (1.0 g, 85.9% yield) which was used directly in the next step without further isolation and characterization. LC/MS: mass calcd, for C₁₂H₂₇NO₂Si: 245.18, found: m/z=246.10 [M+H]⁺.

### Step 4: Synthesis of (S)-3-((tert-butyldimethylsilyl)oxy)-1-(2-chloroethyl)pyrrolidine

To a solution of 2-[(3S)-3-[(tert-butyldimethylsilyl)oxy]pyrrolidin-1-yl]ethanol (1.0 g, 4.07 mmol) in DCM (10 mL) were added Et₃N (1.21 mL, 825 mg, 8.15 mmol, 2.0 eq.) and solid TsCl (1.17 g, 6.11 mmol, 1.5 eq.). The reaction mixture was stirred at rt for 5 h. The reaction mixture was diluted with a saturated aqueous NH₄Cl solution (30 mL) and extracted with DCM (3×50 mL). The combined organic phases were dried over Na₂SO₄, filtered, and the filtrate was concentrated under reduced pressure using a rotary evaporator to afford (3S)-3-[(tert-butyldimethylsilyl)oxy]-1-(2-chloroethyl)-pyrrolidine as a yellow oil (1.0 g, 83.7% yield) which was used directly in the next step without further isolation and characterization. LC/MS: mass calcd. for: C₁₂H₂₆ClNOSi: 263.14, found: m/z=264.00 [M+H]⁺.

### Step 5: Synthesis of N—((R)-1-(2-((S)-3-((tert-butyldimethylsilyl)oxy)pyrrolidin-1-yl)ethyl)piperidin-3-yl)-6-chloro-5-methylpyridazin-3-amine

A brown suspension of (R)-6-chloro-5-methyl-N-(piperidin-3-yl)pyridazin-3-amine hydrochloride (200 mg, 0.76 mmol, 1.0 eq.), (S)-3-((tert-butyldimethylsilyl)oxy)-1-(2-chloroethyl)pyrrolidine (401 mg, 1.52 mmol, 2.0 eq.), NaI (228 mg, 1.52 mmol, 2.0 eq.) and DIEA (398 μL, 295 mg, 2.28 mmol, 3.0 eq.) in ACN (4 mL) was stirred at 80° C. for 2 h. After the reaction mixture cooled to r.t, EtOAc (20 mL) and water (10 mL) were added to the crude reaction mixture. After separation of the phases, the aqueous phase was extracted with EtOAc (2×20 mL). The combined organic phases were washed with brine (2×20 mL), dried over Na₂SO₄, filtered and the filtrate concentrated under reduced pressure using a rotary evaporator. The residue was submitted to silica gel column chromatography eluting with DCM/MeOH gradients (0~5%) to afford N—((R)-1-(2-((S)-3-((tert-butyldimethylsilyl)oxy)pyrrolidin-1-yl)ethyl)piperidin-3-yl)-6-chloro-5-methylpyridazin-3-amine as yellow oil (160 mg, 36.4% yield) after evaporation of the combined product containing fractions under reduced pressure using a rotary evaporator. LC/MS: mass calcd for C₂₂H₄₀ClN₅OSi: 453.27, found: m/z=454.25 [M+H]⁺.

### Step 6: Synthesis of (S)-1-(2-((R)-3-((6-chloro-5-methylpyridazin-3-yl)amino)piperidin-1-yl)ethyl)pyrrolidin-3-ol

A solution of HCl (2 mL, 8.0 mmol, 4 M in 1,4-dioxane) was added to a brown solution of N—((R)-1-(2-((S)-3-((tert-butyldimethylsilyl)oxy)pyrrolidin-1-yl)ethyl)piperidin-3-yl)-6-chloro-5-methylpyridazin-3-amine (200 mg, 0.44 mmol) dissolved in DCM (2 mL) and the reaction mixture was stirred at rt for 3 h resulting in a suspension. After removal of solvents under reduced pressure using a rotary evaporator, (S)-1-(2-((R)-3-((6-chloro-5-methylpyridazin-3-yl)amino)piperidin-1-yl)ethyl)pyrrolidin-3-ol was obtained as a brown oil (100 mg, 63.0% yield) which was used directly without further isolation and characterization. LC/MS: mass calcd for C₁₆H₂₆ClN₅O: 339.18, found: m/z=340.20 [M+H]⁺.

### Step 7: Synthesis of (S)-1-(2-((R)-3-((6-(2-hydroxy-4-(trifluoromethyl)phenyl)-5-methylpyridazin-3-yl)amino)piperidin-1-yl)ethyl)pyrrolidin-3-ol (4)

A solution of (S)-1-(2-((R)-3-((6-chloro-5-methylpyridazin-3-yl)amino)piperidin-1-yl)ethyl)pyrrolidin-3-ol (310 mg, 0.91 mmol, 1.0 eq.), (2-hydroxy-4-(trifluoromethyl)phenyl) boronic acid (282 mg, 1.37 mmol, 1.5 eq.), Pd(PPh₃₃)₄ (32 mg, 0.027 mmol, 0.03 eq.) and NaHCO₃ (153 mg, 1.82 mmol, 2.0 eq.) in dioxane (4 mL) and H₂O (2 mL) was stirred in a sealed tube at 150° C. under an atmosphere of N₂ for 1 h. After filtration and concentration of the filtrate under reduced pressure using a rotary evaporator, the residue was submitted to silica gel column purification eluting with DCM/MeOH gradients (0~10%) to afford giving a yellow oil which was further purified by preparative RP-HPLC using the following conditions: Column: Xselect CSH C18 OBD Column 30*150 mm 5 μm; Mobile Phase A: Water (0.1 vol-% FA), Mobile Phase B: ACN; Flow rate: 60 mL/min; Gradient: 3% B to 21% B in 7 min, 21% B; Wavelength: 254 nm; RT1 (min): 5.38; After lyophilization of the combined fractions, the target compound(S)-1-(2-((R)-3-((6-(2-hydroxy-4-(trifluoromethyl)phenyl)-5-methylpyridazin-3-yl)amino)piperidin-1-yl)ethyl)pyrrolidin-3-ol (4) formate salt was obtained as a white solid (26 mg, 5.5% yield). LC/MS: mass calcd for C₂₄H₃₂F₃N₅O₄: 465.24, found: m/z=466.20 [M+H]⁺. ¹H NMR (400 MHz, Methanol-d₄): δ 7.79-7.37 (m, 1H), 7.30-7.23 (m, 1H), 7.26-7.17 (m, 1H), 6.85-6.81 (m, 1H), 4.61-4.54 (m, 1H), 4.10-4.06 (m, 1H), 3.73-3.61 (m, 1H), 3.55-3.35 (m, 5H), 3.24-3.14 (m, 1H), 2.94-2.82 (m, 2H), 2.69-2.58 (m, 1H), 2.41-2.23 (m, 2H), 2.15 (s, 3H), 2.12-1.74 (m, 5H), 1.52-1.45 (m, 1H) ppm.

### Example 5:4-Fluoro-1-(2-((R)-3-((6-(2-hydroxy-4-(trifluoromethyl)phenyl)-5-methylpyridazin-3-yl)amino)piperidin-1-yl)ethyl)pyrrolidin-3-ol (5)

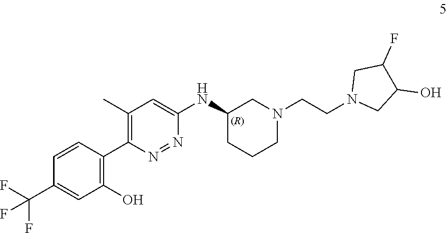

### Synthetic Scheme:

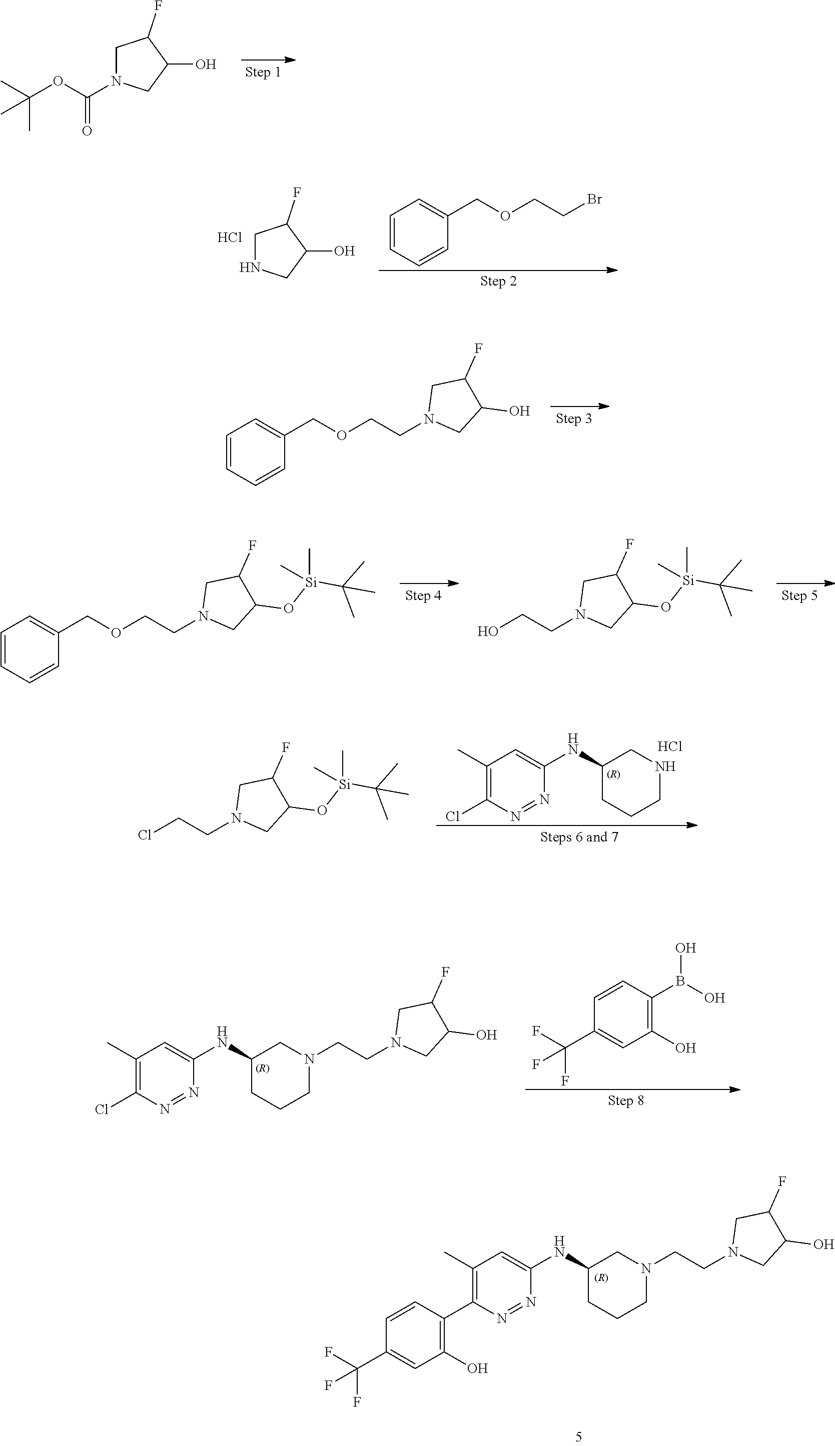

### Step 1: Synthesis of 4-fluoropyrrolidin-3-ol hydrochloride

To a solution of tert-butyl 3-fluoro-4-hydroxypyrrolidine-1-carboxylate (3.00 g, 14.62 mmol) in DCM (15 mL) was added HCl (15 mL, 60 mmol, 4 M in 1,4-dioxane) at 0° C. The resulting solution was stirred at room temperature for 2 h. The reaction mixture was concentrated under reduced pressure using a rotary evaporator to afford 4-fluoropyrrolidin-3-ol hydrochloride as a brown solid (2.00 g, 87.0% yield). LC/MS: mass calcd for C₄H₈FNO: 105.06, found: m/z=106.00 [M+H]⁺.

### Step 2: Synthesis of 1-(2-(benzyloxy)ethyl)-4-fluoropyrrolidin-3-ol

To a solution of [(2-bromoethoxy)methyl]benzene (2.00 g, 9.30 mmol, 1.0 eq.) and 4-fluoropyrrolidin-3-ol hydrochloride (1.45 g, 10.23 mmol, 1.1 eq.) in ACN (50 mL) was added K₂CO₃ (3.86 g, 27.9 mmol). The reaction mixture was stirred at 80° for 2 h. The reaction mixture was diluted with a saturated NH₄Cl aqueous solution (50 mL) and the aqueous phase extracted with DCM (3×100 mL). The combined organic phases were dried over Na₂SO₄, filtered, and the filtrate was concentrated under reduced pressure using a rotary evaporator to afford 1-[2-(benzyloxy)ethyl]-4-fluoropyrrolidin-3-ol as a brown oil (2.00 g, 67.4% yield). LC/MS: mass calcd for C₁₃H₁₈FNO₂: 239.13, found: m/z=240.00 [M+H]⁺.

### Step 3: Synthesis of 1-(2-(benzyloxy)ethyl)-3-((tert-butyldimethylsilyl)oxy)-4-fluoropyrrolidine

To a solution of 1-[2-(benzyloxy)ethyl]-4-fluoropyrrolidin-3-ol (1.90 g, 7.94 mmol) in DCM (20 mL) were added solid TBSCl (1.44 g, 9.53 mmol) and 1H-imidazole (1.08 g, 15.88 mmol). The reaction mixture was stirred at rt for overnight. The reaction mixture was concentrated and the residue was submitted to silica gel column purification eluting with PE/ethyl ether (0~50%) to afford 1-[2-(benzyloxy)ethyl]-3-[(tert-butyldimethylsilyl)oxy]-4-fluoropyrrolidine as a yellow oil (1.60 g, 51.3% yield). LC/MS: mass calcd for C₁₉H₃₂FNO₂Si: 353.22, found: m/z=354.10 [M+H]⁺.

### Step 4: Synthesis of 2-(3-((tert-butyldimethylsilyl)oxy)-4-fluoropyrrolidin-1-yl)ethan-1-ol

To a solution of 1-[2-(benzyloxy)ethyl]-3-[(tert-butyldimethylsilyl)oxy]-4-fluoropyrrolidine (800 mg, 2.26 mmol) in ethyl acetate/t-BuOH (16 mL, 1:1, v/v) was added Pd/C (800 mg, 10 wt-% Pd/C). The resulting suspension was stirred at 60° C. for overnight under an atmosphere of hydrogen atmosphere at atmospheric pressure. After filtration through silica gel, the solution was concentrated under reduced pressure using a rotary evaporator to afford 2-{3-[(tert-butyldimethylsilyl)oxy]-4-fluoropyrrolidin-1-yl}ethanol as a yellow oil (600 mg, 85.6% yield). LC/MS: mass calcd for C₁₂H₂₆FNO₂Si: 263.17, found: m/z=264.05 [M+H]⁺.

### Step 5: Synthesis of 3-((tert-butyldimethylsilyl)oxy)-1-(2-chloroethyl)-4-fluoropyrrolidine

To a solution of 2-{3-[(tert-butyldimethylsilyl)oxy]-4-fluoropyrrolidin-1-yl}ethanol (580 mg, 2.20 mmol) in DCM (6 mL) were added TEA (614 μL, 446 mg, 4.40 mmol, 2.0 eq.) and solid TsCl (504 mg, 2.64 mmol, 1.2 eq.). The reaction mixture was stirred at rt for 5 h and was diluted with a saturated aqueous NH₄Cl solution (30 mL). The aqueous phase was extracted with DCM (3×50 mL). The combined organic phases were dried over Na₂SO₄, filtered and the filtrate was concentrated under reduced pressure using a rotary evaporator to afford 3-[(tert-butyldimethylsilyl)oxy]-1-(2-chloroethyl)-4-fluoropyrrolidine as a yellow oil (600 mg, 77.3% yield). The crude was used in next step without further purification and characterization. LC/MS: mass calcd for: C₁₂H₂₅ClFNOSi: 281.14, found: m/z=282.05 [M+H]⁺.

### Steps 6 and 7: Synthesis of 1-(2-((R)-3-((6-chloro-5-methylpyridazin-3-yl)amino)piperidin-1-yl)ethyl)-4-fluoropyrrolidin-3-ol

To a solution of 6-chloro-5-methyl-N-[(3R)-piperidin-3-yl]pyridazin-3-amine hydrochloride (580 mg, 2.20 mmol) and 3-[(tert-butyldimethylsilyl)oxy]-1-(2-chloroethyl)-4-fluoropyrrolidine (746 mg, 2.65 mmol, 1.2 eq.) in ACN (6 mL) were added NaI (661 mg, 1.41 mmol, 0.65 eq.) and DIEA (1.14 mL, 846 mg, 6.612 mmol, 3.0 eq.). The resulting suspension was stirred at 80° C. for 2 h. After the reaction mixture cooled down to room temperature, it was concentrated under reduced pressure using a rotary evaporator. The residue was submitted to reverse phase column chromatography (0~60% ACN/water (0.05% vol-TFA)) to afford a yellow oil. Under those purification conditions, the TBDMS group was simultaneously removed during lyophilization of combined product containing fractions due to the presence of TFA (0.05 vol-%) in the eluate. The oil obtained was subjected to silica gel column chromatography eluting with DCM/MeOH (0~20%) to afford 1-{2-[(3R)-3-[(6-chloro-5-methylpyridazin-3-yl)amino]piperidin-1-yl]ethyl}-4-fluoropyrrolidin-3-ol after combining the product containing fractions under reduced pressure using a rotary evaporator as a yellow oil (150 mg, 15.2% yield). LC/MS: mass calcd for C₁₆H₂₅ClFN₅O: 357.17, found: m/z=358.05 [M+H]⁺.

### Steps 8: Synthesis of 4-fluoro-1-(2-((R)-3-((6-(2-hydroxy-4-(trifluoromethyl)phenyl)-5-methylpyridazin-3-yl)amino)piperidin-1-yl)ethyl)pyrrolidin-3-ol (5)

To a solution of 1-{2-[(3R)-3-[(6-chloro-5-methylpyridazin-3-yl)amino]piperidin-1-yl]ethyl}-4-fluoropyrrolidin-3-ol (150 mg, 0.42 mmol,) in dioxane/H₂O (3 mL, 2:1, v/v)) were added 2-hydroxy-4-(trifluoromethyl)phenylboronic acid (139 mg, 0.67 mmol, 1.6 eq.), Pd(PPh₃)₄ (15 mg, 0.013 mmol, 0.03 eq.) and NaHCO₃ (106 mg, 1.26 mmol, 3.0 eq.). The resulting reaction mixture was stirred in a sealed tube at 150° C. for 1 h under an atmosphere of N₂. After the reaction mixture was cooled down to rt, the reaction mixture was concentrated under reduced pressure using a rotary evaporator. The residue was submitted to silica gel column purification eluting with DCM/MeOH (0~20%) to afford the crude target compound as a yellow oil (70 mg). The oil obtained was further purified by preparative RP-HPLC using the following conditions: Column: XBridge Prep OBD C18 Column, 30*150 mm, 5 μm; Mobile Phase A: Water+0.1 vol-% FA, Mobile Phase B: ACN; Flow rate: 60 mL/min; Gradient: 5% B to 22% B in 7 min; Wavelength: 254 nm; RT1 (min): 4.92. After lyophilization of the combined product containing fractions, the target compound 4-fluoro-1-{2-[(3R)-3-({6-[2-hydroxy-4-(trifluoromethyl)phenyl]-5-methylpyridazin-3-yl}amino)piperidin-1-yl]ethyl}pyrrolidin-3-ol (5) formate salt was obtained as an off-white solid (4.7 mg, 2.3% yield). LC/MS: mass calcd for C₂₃H₂₉F₄N₅O₂: 483.23, found: m/z=484.20 [M+H]⁺. ¹H NMR (400 MHz, Methanol-d₄) δ 7.43-7.40 (m, 1H), 7.27-7.20 (m, 2H), 6.87 (s, 1H), 5.08-5.04 (m, 1H), 4.88-4.83 (m, 1H), 4.42-4.24 (m, 2H), 3.57-3.37 (m, 2H), 3.28-3.08 (m, 7H), 3.02-2.56 (m, 2H), 2.17-2.01 (m, 5H), 1.93-1.88 (m, 1H), 1.70-1.62 (m, 1H) ppm. ¹⁹F NMR (376 MHz, Methanol-d₄): δ−64.26, −180.21 ppm.

### Example 6: (R)-1-(2-(3-((6-(2-Hydroxy-6-methyl-4-(trifluoromethyl)phenyl)pyridazin-3-yl)amino)piperidin-1-yl)ethyl)piperidin-4-ol (6)

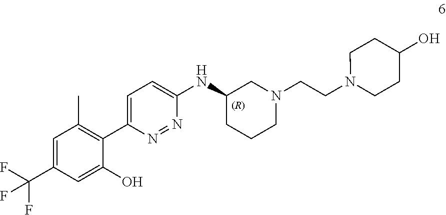

### Synthetic Scheme:

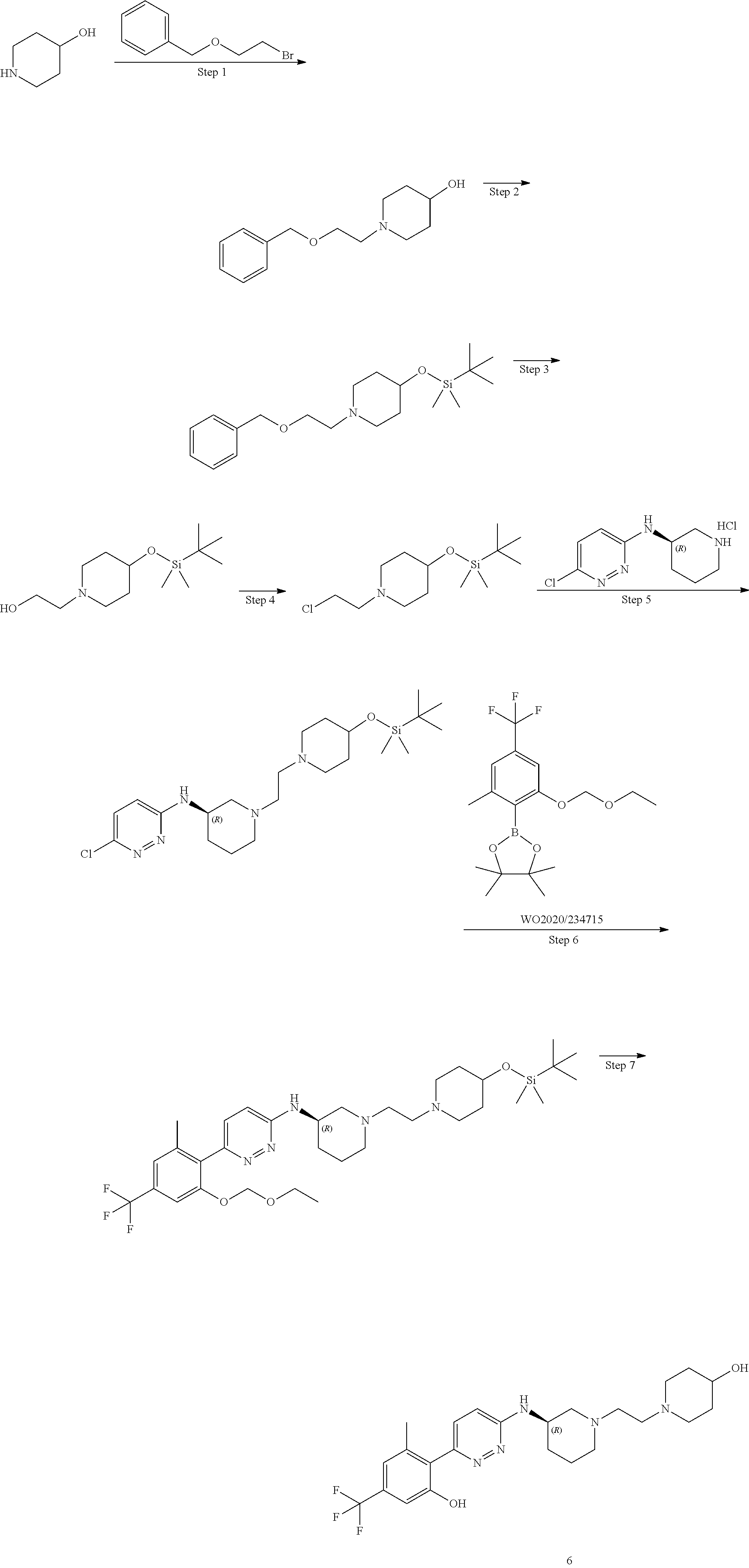

### Step 1: Synthesis of 1-(2-(benzyloxy)ethyl)piperidin-4-ol

To a solution of [(2-bromoethoxy)methyl]benzene (1.47 mL, 2 g, 9.30 mmol, 1.0 eq.) and piperidin-4-ol (1.03 g, 10.2 mmol, 1.1 eq.) in ACN (30 mL) was added K₂CO₃ (3.86 g, 27.9 mmol). The reaction mixture was stirred at 80° C. for 2 h. The reaction mixture was diluted with a saturated aqueous NH₄Cl solution (50 mL) and extracted with DCM (3×100 mL). The combined organic phases were dried over Na₂SO₄, filtered off, and the filtrate was concentrated under reduced pressure using a rotary evaporator to afford 1-[2-(benzyloxy)ethyl]piperidin-4-ol as a yellow oil (2.0 g, 82.3% yield). LC/MS: mass calcd for C₁₄H₂₁NO₂: 235.16, found: m/z=236.05 [M+H]⁺.

### Step 2: Synthesis of 1-(2-(benzyloxy)ethyl)-4-((tert-butyldimethylsilyl)oxy)piperidine

To a solution of 1-[2-(benzyloxy)ethyl]piperidin-4-ol (2 g, 8.50 mmol) in DCM (25 mL) were added TBSCl (1.54 g, 10.20 mmol, 1.2 eq.) and 1H-imidazole (1.16 g, 17.0 mmol, 2.0 eq.). The reaction mixture was stirred at rt overnight. The reaction mixture was concentrated under reduced pressure using a rotary evaporator and the residue was submitted to silica gel column chromatography eluting using a PE/ethyl ether gradient (0~100%) to afford 1-[2-(benzyloxy)ethyl]-4-[(tert-butyldimethylsilyl)oxy]piperidine as a yellow oil (1.2 g, 36.4% yield). LC/MS: mass calcd for C₂₀H₃₅NO₂Si: 349.24, found: m/z=350.20 [M+H]⁺.

### Step 3: Synthesis of 2-(4-((tert-butyldimethylsilyl)oxy) piperidin-1-yl)ethan-1-ol

To a solution of 1-[2-(benzyloxy)ethyl]-4-[(tert-butyldimethylsilyl)oxy]piperidine (1.2 g, 3.43 mmol) in 30 mL of a mixture of EtOAc/t-BuOH (1:1, v/v)) was added solid 10 wt-% Pd/C (1.2 g). The resulting suspension was stirred at 50° C. for overnight under an atmosphere of H₂ at atmospheric pressure. After filtration through silica gel, the filtrate was concentrated under reduced pressure to afford 2-{4-[(tert-butyldimethylsilyl)oxy]piperidin-1-yl}ethanol as a yellow oil (900 mg, 88.9% yield). LC/MS: mass calcd for C₁₃H₂₉NO₂Si: 259.20, found: m/z=260.10 [M+H]⁺.

### Step Synthesis of 4-((tert-butyldimethylsilyl)oxy)-1-(2-chloroethyl)piperidine

To a solution of 2-{4-[(tert-butyldimethylsilyl)oxy]piperidin-1-yl}ethanol (900 mg, 3.47 mmol) in DCM (10 mL) were added Et₃N (967 μL, 702 mg, 6.94 mmol, 2.0 eq.) and solid TsCl (794 mg, 4.16 mmol, 1.2 eq.). The reaction mixture was stirred at rt overnight. The reaction mixture was diluted with a saturated aqueous NH₄Cl solution (30 mL) and the aqueous phase was extracted with DCM (3×50 mL). The combined organic phases were dried over Na₂SO₄, filtered, and the filtrate was concentrated under reduced pressure using a rotary evaporator to afford 4-[(tert-butyldimethylsilyl)oxy]-1-(2-chloroethyl)piperidine as a yellow oil (900 mg, 74.7% yield). LC/MS: mass calcd for: C₁₃H₂₈ClNOSi: 277.16, found: m/z=278.10 [M+H]⁺.

### Step 5: Synthesis of (R)—N-(1-(2-(4-((tert-butyldimethylsilyl)oxy) piperidin-1-yl)ethyl)piperidin-3-yl)-6-chloropyridazin-3-amine

To a solution of 6-chloro-N-[(3R)-piperidin-3-yl]pyridazin-3-amine hydrochloride (300 mg, 1.20 mmol) and 4-[(tert-butyldimethylsilyl)oxy]-1-(2-chloroethyl)piperidine (402 mg, 1.45 mmol, 1.2 eq.) in ACN (5 mL) were added NaI (361 mg, 2.41 mmol, 2.0 eq.) and DIEA (629 μL, 467 mg, 3.61 mmol, 3.0 eq.). The resulting suspension was stirred at 80° C. for 2 h. After the reaction mixture cooled down to rt, the reaction mixture was washed with a saturated aqueous NH₄Cl solution (20 mL) and extracted with DCM (3×50 mL). The combined organic phases were dried over Na₂SO₄, filtered, and the filtrated concentrated under reduced pressure using a rotary evaporator. The residue was subjected to silica gel column purification eluting with DCM/MeOH gradients (0~20%) to afford N-[(3R)-1-(2-{4-[(tert-butyldimethylsilyl)oxy]piperidin-1-yl}ethyl)piperidin-3-yl]-6-chloropyridazin-3-amine as a yellow oil (300 mg, 49.4% yield). LC/MS: mass calcd for C₂₂H₄₀ClNOSi: 453.27, found: m/z=454.20 [M+H]⁺.

### Step 6: Synthesis of N-[(3R)-1-(2-{4-[(tert-butyldimethylsilyl)oxy]piperidin-1-yl}ethyl)piperidin-3-yl]-6-[2-(ethoxymethoxy)-6-methyl-4-(trifluoromethyl)phenyl]pyridazin-3-amine

To a solution of N-[(3R)-1-(2-{4-[(tert-butyldimethylsilyl)oxy]piperidin-1-yl}ethyl)piperidin-3-yl]-6-chloropyridazin-3-amine (150 mg, 0.33 mmol, 1.0 eq.) in 2.4 mL of a mixture of dioxane/H₂O (2:1, v/v)) were added 2-[2-(ethoxymethoxy)-6-methyl-4-(trifluoromethyl)phenyl]-4,4,5,5-tetramethyl-1,3,2-dioxaborolane (prepared according to the international patent application publication WO2020/234715) (238 mg, 0.66 mmol, 2.0 eq.), Pd(PPh₃)₄ (12 mg, 0.010 mmol, 0.03 eq.) and NaHCO₃ (84 mg, 0.990 mmol, 3.0 eq.). The resulting reaction mixture was stirred in a sealed tube at 150° C. for 1 h under an atmosphere of N₂. After the reaction mixture cooled down to rt, the reaction mixture was concentrated under reduced pressure using a rotary evaporator The residue was subjected to silica gel column purification eluting with DCM/MeOH gradients (0~20%) to afford N-[(3R)-1-(2-{4-[(tert-butyldimethylsilyl)oxy]piperidin-1-yl}ethyl)piperidin-3-yl]-6-[2-(ethoxymethoxy)-6-methyl-4-(trifluoromethyl)phenyl]pyridazin-3-amine as a yellow oil (100 mg, 41.8% yield). LC/MS: mass calcd for C₃₃H₅₂F₃N₅O₃Si: 651.38, found: m/z=652.40 [M+H]⁺.

### Step 7: Synthesis of 1-{2-[(3R)-3-({6-[2-hydroxy-6-methyl-4-(trifluoromethyl)phenyl]pyridazin-3-yl}amino)piperidin-1-yl]ethyl}piperidin-4-ol (6)

To a solution of N-[(3R)-1-(2-{4-[(tert-butyldimethylsilyl)oxy]piperidin-1-yl}ethyl)piperidin-3-yl]-6-[2-(ethoxymethoxy)-6-methyl-4-(trifluoromethyl)phenyl]pyridazin-3-amine (120 mg, 0.18 mmol) in DCM (1.5 mL) was added TFA (1.5 mL) at 0° C. The resulting solution was stirred at rt for 2 h. The reaction mixture was concentrated under reduced pressure using a rotary evaporator. The obtained residue was subjected to preparative RP-HPLC using the following conditions: Column: XBridge Prep OBD C18 Column, 30*150 mm, 5 μm; Mobile Phase A: Water+0.1 vol-% FA, Mobile Phase B: ACN; Flow rate: 25 mL/min; Gradient: 39% B to 59% B in 7 min; Wavelength: 254 nm; RT1: 4.6 min. After lyophilization of the combined product containing fractions the target compound 1-{2-[(3R)-3-({6-[2-hydroxy-6-methyl-4-(trifluoromethyl)phenyl]pyridazin-3-yl}amino)piperidin-1-yl]ethyl}piperidin-4-ol (6) was obtained as a colorless solid (2.6 mg, 2.8% yield). LC/MS: mass calcd for C₂₄H₃₂F₃N₅O₂: 479.25, found: m/z=480.25 [M+H]⁺. ¹H NMR (400 MHz, Methanol-d₄): δ 7.34-7.32 (m, 1H), 7.09-7.06 (m, 1H), 7.02-6.96 (m, 2H), 4.17-4.15 (m, 1H), 3.68-3.65 (m, 1H), 3.15-3.08 (m, 1H), 2.96-2.93 (m, 2H), 2.82-2.72 (m, 1H), 2.69-2.59 (m, 4H), 2.43-2.22 (m, 3H), 2.21-2.00 (m, 4H), 1.99-1.91 (m, 1H), 1.88-1.83 (m, 3H), 1.74-1.63 (m, 3H), 1.52-1.43 (m, 1H) ppm. ¹⁹F NMR (376 MHz, Methanol-d₄): δ −64.41 ppm.

### Example 7: (R)-1-(2-(3-((6-(2-Hydroxy-4-(trifluoromethyl)phenyl)-5-methylpyridazin-3-yl)amino)piperidin-1-yl)ethyl)piperidin-4-ol (7)

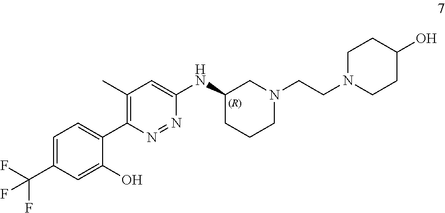

### Synthetic Scheme:

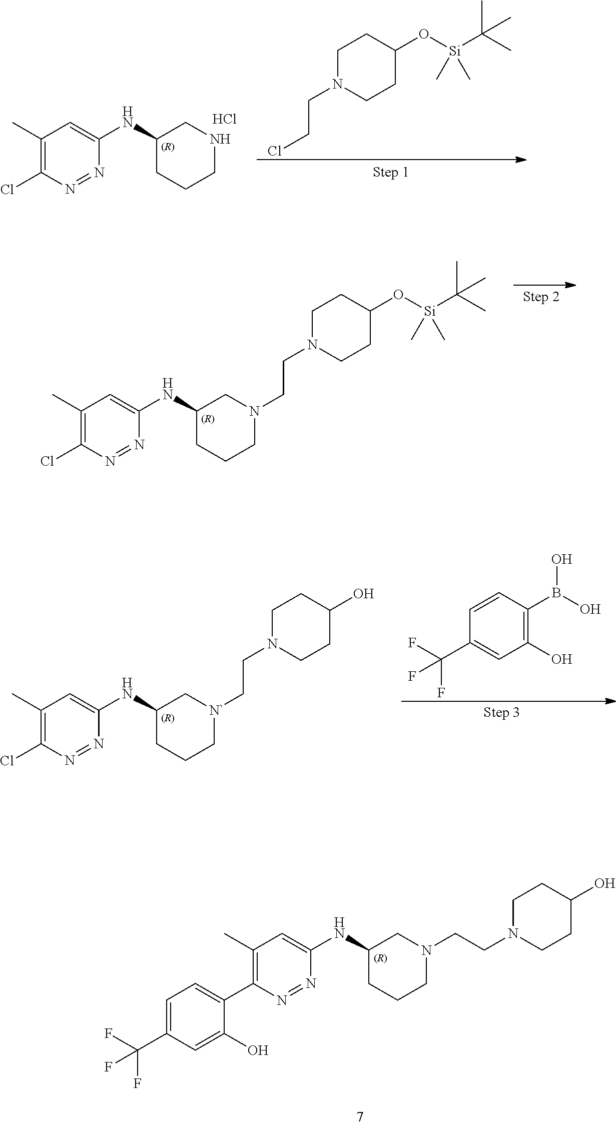

### Step 1: Synthesis of (R)—N-(1-(2-(4-((tert-butyldimethylsilyl)oxy) piperidin-1-yl)ethyl)piperidin-3-yl)-6-chloro-5-methylpyridazin-3-amine

A brown suspension of (R)-6-chloro-5-methyl-N-(piperidin-3-yl)pyridazin-3-amine hydrochloride (300 mg, 1.14 mmol 1.0 eq.), 4-((tert-butyldimethylsilyl)oxy)-1-(2-chloroethyl)piperidine (634 mg, 2.28 mmol, 2.0 eq.), NaI (342 mg, 2.28 mmol, 2.2 eq.) and DIEA (593 μL, 440 mg, 3.42 mmol, 3.0 eq.) in ACN (3 mL) was stirred at 80° C. for 2 h. After the reaction mixture cooled to rt, EtOAc (20 mL) and water (10 mL) were added to the suspension. After separation of the phases, the aqueous phase was extracted with EtOAc (2×20 mL). The combined organic phases were washed with brine (2×20 mL), dried over Na₂SO₄, filtered, and the filtrate was concentrated under reduced pressure using a rotary evaporator. The residue was subjected to silica gel column chromatography eluting with EtOAc/PE gradients (0~70%) to afford (R)—N-(1-(2-(4-((tert-butyldimethylsilyl)oxy) piperidin-1-yl)ethyl)piperidin-3-yl)-6-chloro-5-methylpyridazin-3-amine as yellow oil (200 mg, 37.5% yield). LC/MS: mass calcd for C₂₃H₄₂ClN₅OSi: 467.28, found: m/z=468.30 [M+H]⁺.

### Step 2: Synthesis of (R)-1-(2-(3-((6-chloro-5-methylpyridazin-3-yl)amino)piperidin-1-yl)ethyl)piperidin-4-ol

A solution of HCl (3 mL, 12 mmol, 4 HCl in 1,4-dioxane) was added to a brown solution of (R)—N-(1-(2-(4-((tert-butyldimethylsilyl)oxy) piperidin-1-yl)ethyl)piperidin-3-yl)-6-chloro-5-methylpyridazin-3-amine (200 mg, 0.43 mmol) in DCM (3 mL) to result in a brown solution which was stirred at rt for 3 h forming a suspension. After removal of solvents under reduced pressure using a rotary evaporator, (R)-1-(2-(3-((6-chloro-5-methylpyridazin-3-yl)amino)piperidin-1-yl)ethyl)piperidin-4-ol was obtained as a yellow oil (140 mg, 68.6% yield) which was used without further purification and characterization. LC/MS: mass calcd for C₁₇H₂₈ClN₅O: 353.20, found: m/z=354.25 [M+H]⁺.

### Step 3: Synthesis of (R)-1-(2-(3-((6-(2-hydroxy-4-(trifluoromethyl)phenyl)-5-methylpyridazin-3-yl)amino)piperidin-1-yl)ethyl)piperidin-4-ol (7)

A solution of 1-{2-[(3R)-3-[(6-chloro-5-methylpyridazin-3-yl)amino]piperidin-1-yl]ethyl}piperidin-4-ol (250 mg, 0.71 mmol, 1.0 eq.), (2-hydroxy-4-(trifluoromethyl)phenyl) boronic acid (218 mg, 1.06 mmol, 1.5 eq. mmol), Pd(PPh₃)₄ (24 mg, 0.021 mmol, 0.03 eq.) and NaHCO₃ (119 mg, 1.41 mmol, 2.0 eq.) in 4.5 mL of a mixture of 1,4-dioxane and water (2:1, v/v) was stirred in a sealed tube at 150° C. under an atmosphere of N₂ for 1 h. After filtration of the solids and concentration under reduced pressure, the residue was subjected to silica gel column chromatography eluting with DCM/MeOH gradients (0~10%) to yield a yellow oil after concentration of the product containing fractions under reduced pressure using a rotary evaporator, which was further purified by preparative RP-HPLC using the following conditions: Column: Xselect CSH C18 OBD Column 30*150 mm 5 μm; Mobile Phase A: Water (0.1 vol-% FA), Mobile Phase B: ACN; Flow rate: 60 mL/min; Gradient: 3% B to 18% B in 7 min, 18% B; Wavelength: 254 nm; RT1: 5.40 min. After lyophilization of the product containing fractions, the target compound (R)-1-(2-(3-((6-(2-hydroxy-4-(trifluoromethyl)phenyl)-5-methylpyridazin-3-yl)amino)piperidin-1-yl)ethyl)piperidin-4-ol (7) formate salt was obtained as a colorless solid (27.6 mg, 7.4% yield). LC/MS: mass calcd for C₂₄H₃₂F₃N₅O₂: 479.25, found: m/z=480.2 [M+H]⁺. ¹H NMR (400 MHz, Methanol-d₄): δ 7.42 (d, J=7.8 Hz, 1H), 7.26 (d, J=7.9 Hz, 1H), 7.20 (s, 1H), 6.84 (s, 1H), 4.20-3.86 (m, 2H), 3.46-3.36 (m, 4H), 3.28-3.24 (m, 1H), 3.16-3.09 (m, 3H), 2.94-2.90 (m, 2H), 2.75-2.67 (m, 1H), 2.40-2.35 (m, 1H), 2.15 (s, 3H), 2.08-2.01 (m, 3H), 1.97-1.72 (m, 4H), 1.55-1.47 (m, 1H) ppm.

### Example 8: (R)-2-(3-((6-(2-Hydroxy-4-(trifluoromethyl)phenyl)-5-methylpyridazin-3-yl)amino)piperidin-1-yl)-1-(3-hydroxyazetidin-1-yl)ethan-1-one (8)

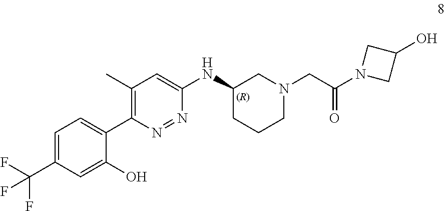

### Synthetic Scheme:

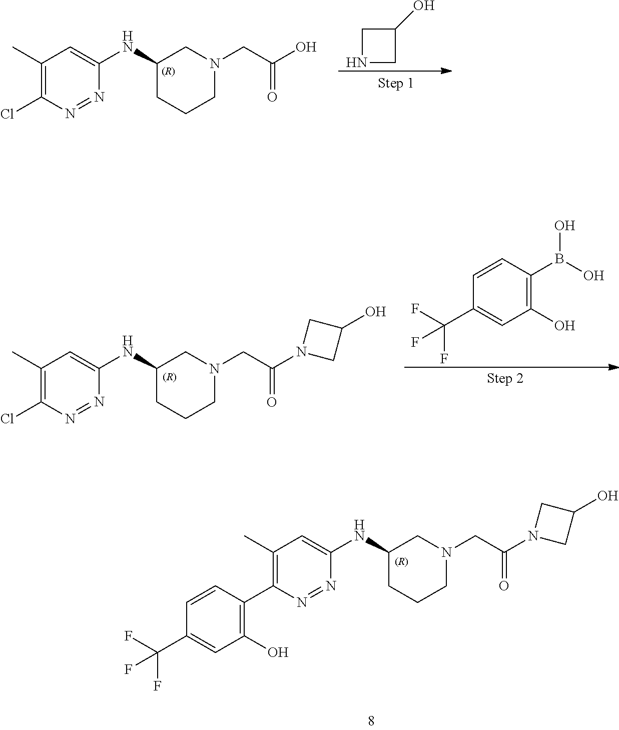

### Step 1: Synthesis of (R)-2-(3-((6-chloro-5-methylpyridazin-3-yl)amino)piperidin-1-yl)-1-(3-hydroxyazetidin-1-yl)ethan-1-one

To a yellow suspension of [(3R)-3-[(6-chloro-5-methylpyridazin-3-yl)amino]piperidin-1-yl]acetic acid (300 mg, 1.054 mmol), azetidine-3-ol hydrochloride (173 mg, 1.58 mmol, 1.5 eq.) and DIEA (1.1 mL, 817 mg, 6.32 mmol, 6.0 eq.) in DMF (3 mL), solid HATU (601 mg, 1.58 mmol, 1.5 eq.) was added portion-wise under stirring giving a yellow suspension. The suspension was stirred at rt for 1 h. The reaction progress was monitored by LC/MS. After concentration under reduced pressure, the suspension was subjected to reverse phase column chromatography eluting with water (10 mmol/L NH₄HCO₃)/ACN gradients (5~60%) to afford 2-[(3R)-3-[(6-chloro-5-methylpyridazin-3-yl)amino]piperidin-1-yl]-1-(3-hydroxyazetidin-1-yl)ethanone as a yellow oil (290 mg, 80.2% yield) after concentration of the product containing fractions by a rotary evaporator under reduced pressure at 60° C. LC/MS: mass calcd for C₁₅H₂₂ClN₅O₂: 339.15, found: m/z=340.30 [M+H]⁺.

### Step 2: Synthesis of (R)-2-(3-((6-(2-hydroxy-4-(trifluoromethyl)phenyl)-5-methylpyridazin-3-yl)amino)piperidin-1-yl)-1-(3-hydroxyazetidin-1-yl)ethan-1-one (8)

A yellow suspension of 2-[(3R)-3-[(6-chloro-5-methylpyridazin-3-yl)amino]piperidin-1-yl]-1-(3-hydroxyazetidin-1-yl)ethanone (290 mg, 0.85 mmol, 1 eq.), 2-hydroxy-4-(trifluoromethyl)phenylboronic acid (281 mg, 1.37 mmol, 1.6 eq.), Pd(PPh₃)₄ (29.6 mg, 0.026 mmol, 0.03 eq.) and NaHCO₃ (215 mg, 2.56 mmol) in 6.6 mL of a mixture of 1,4-dioxane and H₂O (2:1. v/v) was stirred in a sealed tube at 150° C. for 1 h. The reaction was monitored by LC/MS. After cooling to rt, to this suspension were added EtOAc (10 mL) and water (20 mL). After separation of the phases, the aqueous phase was extracted with EtOAc (2×10 mL). The combined organic phases were washed with brine (2×40 mL), dried over Na₂SO₄, filtered, and the filtrate was concentrated under reduced pressure using a rotary evaporator. The residue was subjected to silica gel column chromatography purification to afford the crude target compound as a yellow solid (398 mg). The solid was further purified by preparative RP-HPLC using the following conditions: Column: CHIRALPAK IB N-3, 4.6*100 mm, 3 μm; Mobile Phase B: MEOH (vol-0.1% DEA); Flow rate: 2 mL/min; Gradient: isocratic 10% B; Wavelength: 220 nm. After lyophilization of the combined product containing fractions, the target compound 2-[(3R)-3-({6-[2-hydroxy-4-(trifluoromethyl)phenyl]-5-methylpyridazin-3-yl}amino)piperidin-1-yl]-1-(3-hydroxyazetidin-1-yl) ethenone (8) was obtained (27.5 mg, 6.9% yield). LC/MS: mass calcd for C₂₂H₂₆F₃N₅O₃: 465.20, found: m/z=466.10 [M+H]⁺. ¹H NMR (300 MHz, Methanol-d₄): δ 7.40 (m, 1H), 7.24 (m, 2H), 6.80 (m, 1H), 4.58 (m, 2H), 4.19 (m, 3H), 3.79 (m, 1H), 3.10 (m, 2H), 2.92 (m, 1H), 2.57 (m, 1H), 2.38 (m, 2H), 2.15 (s, 3H), 1.87 (m, 2H), 1.60 (m, 2H). ¹⁹F NMR (282 MHz, Methanol-d₄): δ −64.29 ppm.

### Example 9: 2-[(3R)-3-({6-[2-Hydroxy-4-(trifluoromethyl)phenyl]-5-methylpyridazin-3-yl}amino)piperidin-1-yl]-1-(4-hydroxypiperidin-1-yl)ethanone (9)

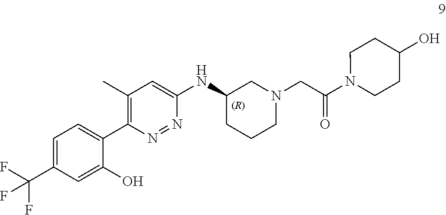

### Synthetic Scheme:

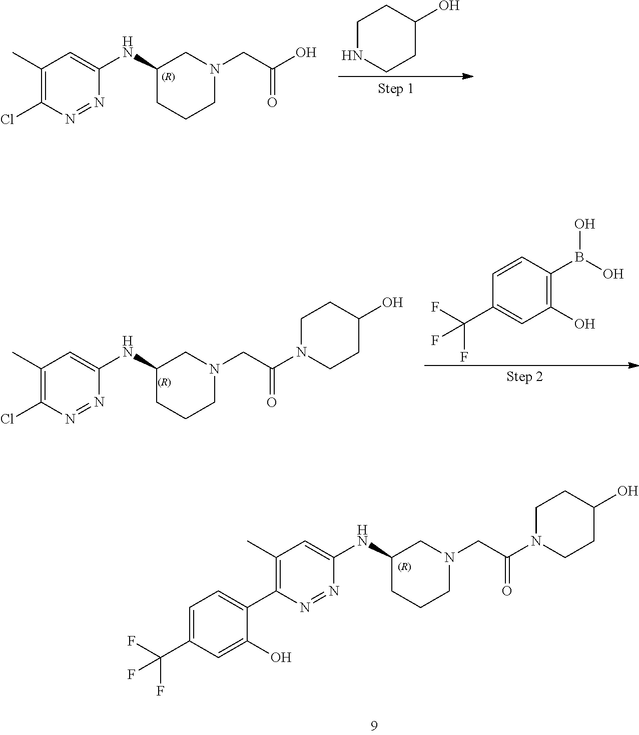

### Step 1: Synthesis of 2-[(3R)-3-[(6-chloro-5-methylpyridazin-3-yl)amino]piperidin-1-yl]-1-(4-hydroxypiperidin-1-yl)ethanone

HATU (321. mg, 0.84 mmol, 1.2 eq.) was added into a colorless solution of [(3R)-3-[(6-chloro-5-methylpyridazin-3-yl)amino]piperidin-1-yl]acetic acid (200 mg, 0.70 mmol), piperidin-4-ol (71.1 mg, 0.70 mmol, 1.0 eq.) and DIEA (367 μL, 272 mg, 2.11 mmol, 3.0 eq.) in DMF (2 mL) affording a slight yellow solution which was stirred at rt for overnight. To this solution EtOAc (20 mL) and water (10 mL) were added. After separation of the phases, the aqueous phase was extracted with EtOAc (2×20 mL). The combined organic phases were washed with brine (1×50 mL), dried over Na₂SO₄, filtered, and the filtrate concentrated under reduced using a rotary evaporator. The residue was subjected to silica gel column chromatography purification to afford 2-[(3R)-3-[(6-chloro-5-methylpyridazin-3-yl)amino]piperidin-1-yl]-1-(4-hydroxypiperidin-1-yl)ethanone as an off-white solid (150 mg, 54.4% yield). LC/MS: mass calcd for C₁₇H₂₆ClN₅O₂: 367.18, found: m/z=368.10 [M+H]⁺.

### Step 2: Synthesis of 2-[(3R)-3-({6-[2-hydroxy-4-(trifluoromethyl)phenyl]-5-methylpyridazin-3-yl}amino)piperidin-1-yl]-1-(4-hydroxypiperidin-1-yl)ethanone (9)

A yellow solution of 2-[(3R)-3-[(6-chloro-5-methylpyridazin-3-yl)amino]piperidin-1-yl]-1-(4-hydroxypiperidin-1-yl)ethanone (150 mg, 0.41 mmol), 2-(4,4,5,5-tetramethyl-1,3,2-dioxaborolan-2-yl)-5-(trifluoromethyl)phenol (176.2 mg, 0.61 mmol, 1.5 eq.), NaHCO₃ (103 mg, 1.22 mmol, 3.0 eq.) in 3.9 mL of a mixture of 1,4-dioxane/H₂O (2:1, v/v)) was stirred until homogeneous. Then, Pd(PPh₃)₄ (14.1 mg, 0.012 mmol, 0.03 eq.) was added and the mixture was stirred under an atmosphere of N₂ in a sealed tube at 150° C. for 1 h. After cooling to rt and concentration under reduced pressure using a rotary evaporator, the residue was subjected to silica gel column purification to afford a yellow solid. The yellow solid was further purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 15% B to 45% B in 9 min, 45% B; Wavelength: 254 nm; RT1: 7 min. After lyophilization of the combined product containing fractions, the target compound 2-[(3R)-3-({6-[2-hydroxy-4-(trifluoromethyl)phenyl]-5-methylpyridazin-3-yl}amino)piperidin-1-yl]-1-(4-hydroxypiperidin-1-yl)ethanone (9) was obtained as a light yellow solid (35 mg, 17.4% yield). LC/MS: mass calcd for C₂₄H₃₀F₃N₅O₃: 493.53, found: m/z=494.40 [M+H]⁺. ¹H NMR (300 MHz, Methanol-d₄): δ 7.51-7.35 (m, 1H), 7.29-7.23 (m, 1H), 7.18 (s, 1H), 6.84-6.75 (m, 1H), 4.26-3.73 (m, 4H), 3.45-3.36 (m, 1H), 3.31-3.08 (m, 3H), 3.07-2.92 (m, 3H), 2.66 (s, 1H), 2.44-2.24 (m, 1H), 2.20-2.09 (m, 3H), 2.02-1.30 (m, 8H) ppm. ¹⁹F NMR (282 MHz, Methanol-d₄): δ −64.28 ppm.

### Example 10: 2-((R)-3-((6-(2-Hydroxy-4-(trifluoromethyl)phenyl)-5-methylpyridazin-3-yl)amino)piperidin-1-yl)-N-((1r,3R)-3-hydroxycyclobutyl)acetamide (10)

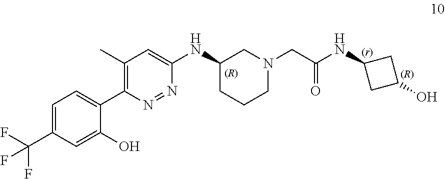

### Synthetic Scheme:

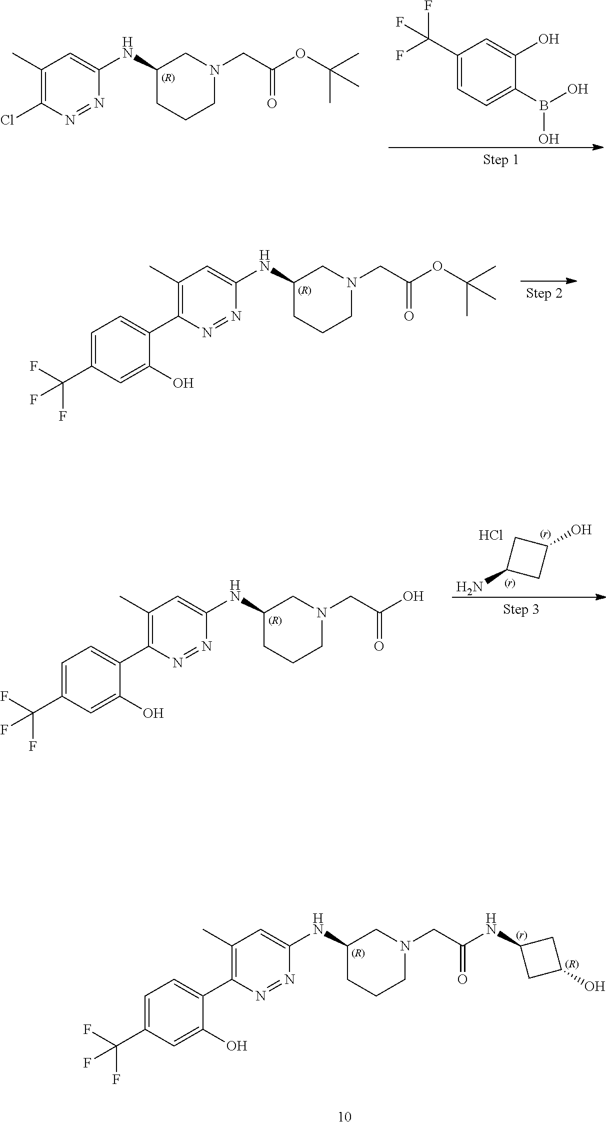

### Step 1: Synthesis of tert-butyl (R)-2-(3-((6-(2-hydroxy-4-(trifluoromethyl)phenyl)-5-methylpyridazin-3-yl)amino)piperidin-1-yl)acetate

To a solution of tert-butyl 2-[(3R)-3-[(6-chloro-5-methylpyridazin-3-yl)amino]piperidin-1-yl]acetate (1.00 g, 2.93 mmol, 1.0 eq.), 2-hydroxy-4-(trifluoromethyl)phenylboronic acid (0.91 g, 4.40 mmol, 1.5 eq.) in a 21 mL of a mixture of 1,4-dioxane/H₂O (2:1, v/v) was added NaHCO₃ (0.74 g, 8.80 mmol, 3.0 eq.) and Pd(PPh₃)₄ (0.1 g, 0.088 mmol, 0.03 eq.). The reaction mixture was stirred in a sealed tube under an atmosphere of N₂ at 150° C. for 1 h. After the reaction mixture cooled down to rt, the reaction was quenched with an aqueous solution of NaCl (20 mL) and further diluted with water (20 mL). The aqueous phase was extracted with EtOAc (3×30 mL). The combined organic phases were washed with brine (2×80 mL), dried over Na₂SO₄, filtered, and the filtrate was concentrated under reduced pressure using a rotary evaporator. This residue was subjected to silica gel column chromatography purification to afford tert-butyl 2-[(3R)-3-({6-[2-hydroxy-4-(trifluoromethyl)phenyl]-5-methylpyridazin-3-yl}amino)piperidin-1-yl]acetate as an off-colorless solid (1.1 g, 75.5% yield). LC/MS: mass calcd for C₂₃H₂₉F₃N₄O₃: 466.22, found: m/z=467.18 [M+H]⁺.

### Step 2: Synthesis of (R)-2-(3-((6-(2-hydroxy-4-(trifluoromethyl)phenyl)-5-methylpyridazin-3-yl)amino)piperidin-1-yl)acetic Acid

To a yellow solution of tert-butyl 2-[(3R)-3-({6-[2-hydroxy-4-(trifluoromethyl)phenyl]-5-methylpyridazin-3-yl}amino)piperidin-1-yl]acetate (1.1 g, 2.54 mmol) in DCM (4 mL), a solution of 4 M in 1,4-dioxane (8 mL, 32 mmol) was added affording a suspension. The suspension was stirred at rt for 4 h. The solvents were removed under reduced pressure using a rotary evaporator to afford (R)-2-(3-((6-(2-hydroxy-4-(trifluoromethyl)phenyl)-5-methylpyridazin-3-yl)amino)piperidin-1-yl)acetic acid as a yellow solid (880 mg, 77.3% yield) which was used directly without further purification and characterization. LC/MS: mass calcd for C₁₉H₂₁F₃N₄O₃: 410.16, found: m/z=411.15 [M+H]⁺.

### Step 3: Synthesis of 2-((R)-3-((6-(2-hydroxy-4-(trifluoromethyl)phenyl)-5-methylpyridazin-3-yl)amino)piperidin-1-yl)-N-((1r,3R)-3-hydroxycyclobutyl)acetamide (10)

BOP (129 mg, 0.29 mmol) was added into a colorless solution of [(3R)-3-({6-[2-hydroxy-4-(trifluoromethyl)phenyl]-5-methylpyridazin-3-yl}amino)piperidin-1-yl]acetic acid (100 mg, 0.24 mmol), (1r,3r)-3-aminocyclobutan-1-ol hydrochloride (36 mg, 0.29 mmol), and DIEA (170 μL, 126 mg, 0.98 mmol) in DMF (3 mL) affording a slight yellow solution. The solution was stirred at rt for 1 h. The solution was subjected to preparative RP-HPLC using the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 25% B to 45% B in 7 min, 45% B; Wavelength: 254 nm; RT1: 6.05 min. After lyophilization of combined product containing fractions, the target compound 2-[(3R)-3-({6-[2-hydroxy-4-(trifluoromethyl)phenyl]-5-methylpyridazin-3-yl}amino)piperidin-1-yl]-N-[(1r,3r)-3-hydroxycyclobutyl]acetamide (10) was obtained as an off-white solid (10.0 mg, 8.6% yield). LC/MS: mass calcd for C₂₃H₂₈F₃N₅O₃: 479.21, found: m/z=480.20 [M+H]⁺. ¹H NMR (300 MHz, Methanol-d₄): δ 7.46-7.37 (m, 1H), 7.28-7.15 (m, 2H), 6.86-6.80 (m, 1H), 4.45-4.33 (m, 2H), 4.26-4.20 (m, 1H), 3.12-2.94 (m, 2H), 2.85-2.79 (m, 1H), 2.55-2.49 (m, 3H), 2.36-2.20 (m, 4H), 2.19-2.13 (m, 3H), 1.92-1.84 (m, 2H), 1.75-1.69 (m, 2H), 1.64-1.58 (m, 2H) ppm. ¹⁹F NMR (282 MHz, Methanol-d₄): δ −64.29 ppm.

### Example 11: 2-((R)-3-((6-(2-Hydroxy-4-(trifluoromethyl)phenyl)-5-methylpyridazin-3-yl)amino)piperidin-1-yl)-1-((S)-3-hydroxypyrrolidin-1-yl)ethan-1-one (11)

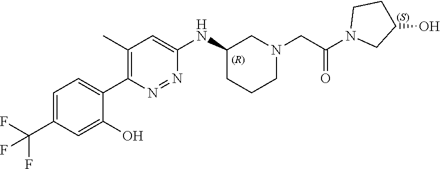

### Synthetic Scheme

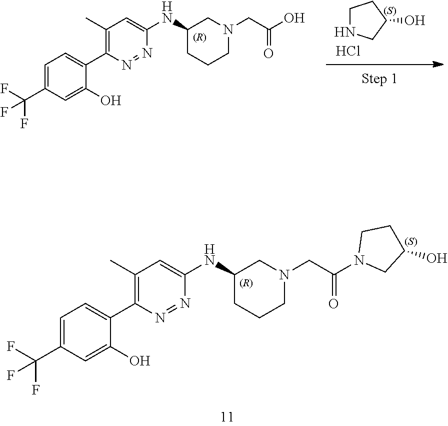

### Step 1: Synthesis of 2-((R)-3-((6-(2-hydroxy-4-(trifluoromethyl)phenyl)-5-methylpyridazin-3-yl)amino)piperidin-1-yl)-1-((S)-3-hydroxypyrrolidin-1-yl)ethan-1-one (11)

In a flask containing [(3R)-3-({6-[2-hydroxy-4-(trifluoromethyl)phenyl]-5-methylpyridazin-3-yl}amino)piperidin-1-yl]acetic acid (prepared as disclosed in Example 10) (2.00 g, 0.50 mmol, 1.0 eq), (S)-pyrrolidin-3-ol hydrochloride (602 mg, 0.50 mmol, 1.0 eq) and EDCI (287.4 mg, 1.5 mmol, 3.0 eq), pyridine (2.0 mL) was added. The reaction mixture was stirred at 50° C. (heating block) overnight under an atmosphere of N₂. After cooling down to rt, the crude reaction mixture was concentrated under reduced pressure using a rotary evaporator to yield the crude reaction product. The crude reaction product was purified by preparative RP-HPLC using ACN/water mixtures acidified with 0.3 vol-% FA (5-95% gradients in 30 min) to afford the target compound 2-((R)-3-((6-(2-hydroxy-4-(trifluoromethyl)phenyl)-5-methylpyridazin-3-yl)amino)piperidin-1-yl)-1-((S)-3-hydroxypyrrolidin-1-yl)ethan-1-one (11) formate salt (30.2 mg, 13.0% yield) after lyophilization of the combined product containing fractions as a colorless solid. LC/MS: m/z=480.15 [M+H]⁺. ¹H NMR (400 MHz, Methanol-d₄): δ 8.33 (s, 1H), 7.38 (d, J=7.9 Hz, 1H), 7.22 (d, J=7.9 Hz, 1H), 7.16 (d, J=1.7 Hz, 1H), 6.83 (s, 1H), 4.45-4.39 (m, 1H), 4.29 (s, 1H), 3.70 (s, 1H), 3.60-3.56 (m, 4H), 3.46 (d, J=2.5 Hz, 2H), 3.09 (s, 1H), 2.76 (s, 2H), 2.14 (s, 3H), 1.98 (s, 4H), 1.82 (s, 1H), 1.67 (s, 1H) ppm.

### Example 12: 2-((R)-3-((6-(2-Hydroxy-4-(trifluoromethyl)phenyl)-5-methylpyridazin-3-yl)amino)piperidin-1-yl)-1-((R)-3-hydroxypyrrolidin-1-yl)ethan-1-one (12)

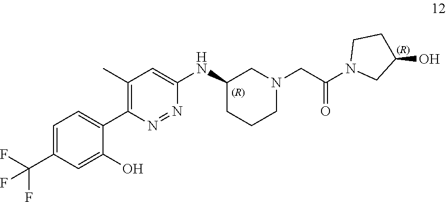

### Synthetic Scheme

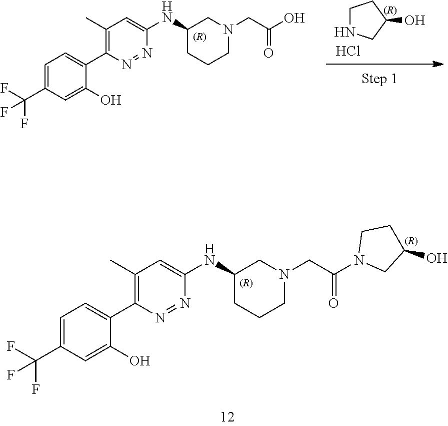

### Step 1: Synthesis of 2-((R)-3-((6-(2-hydroxy-4-(trifluoromethyl)phenyl)-5-methylpyridazin-3-yl)amino)piperidin-1-yl)-1-((R)-3-hydroxypyrrolidin-1-yl)ethan-1-one (12)

In a flask containing [(3R)-3-({6-[2-hydroxy-4-(trifluoromethyl)phenyl]-5-methylpyridazin-3-yl}amino)piperidin-1-yl]acetic acid (prepared as disclosed in Example 10) (500 mg, 1.2 mmol, 1.0 eq), (S)-pyrrolidin-3-ol hydrochloride (151 mg, 1.2 mmol, 1.0 eq) and EDCI (690 mg, 3.6 mmol, 3.0 eq), Pyridine (5.0 mL) was added. The reaction mixture was stirred at 50° C. overnight under an atmosphere of N₂. After cooling down to rt, the reaction mixture was concentrated under reduced pressure using a rotary evaporator to yield the crude reaction mixture. The crude reaction mixture crude was purified by preparative-RP-PLC using ACN/water gradients acidified with 0.3 vol-% FA to afford the target compound 2-((R)-3-((6-(2-hydroxy-4-(trifluoromethyl)phenyl)-5-methylpyridazin-3-yl)amino)piperidin-1-yl)-1-((R)-3-hydroxypyrrolidin-1-yl)ethan-1-one (12) (65.2 mg, 11.2% yield) after lyophilization of the combined product containing fractions as a colorless solid. LC/MS: m/z=480.40 [M+H]⁺. ¹H NMR (400 MHz, Methanol-d₄): ¹H NMR (400 MHz, Methanol-d₄): δ 7.39 (d, J=7.9 Hz, 1H), 7.26-7.20 (m, 1H), 7.16 (d, J=1.7 Hz, 1H), 6.82 (d, J=1.8 Hz, 1H), 4.50-4.43 (m, 1H), 4.25 (d, J=8.7 Hz, 1H), 3.70-3.60 (m, 2H), 3.56 (s, 2H), 3.48 (s, 3H), 3.20-3.14 (m, 1H), 2.85 (s, 1H), 2.68-2.64 (m, 2H), 2.13 (s, 3H), 1.97-1.92 (m, 4H), 1.85-1.57 (m, 2H) ppm.

### Example 13: (R)-2-(3-((6-(2-Hydroxy-4-(trifluoromethyl)phenyl)-5-methylpyridazin-3-yl)amino)piperidin-1-yl)-N-(3-hydroxybicyclo[1.1.1]pentan-1-yl)acetamide (13)

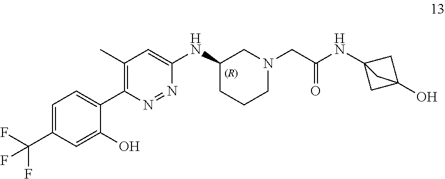

### Synthetic Scheme:

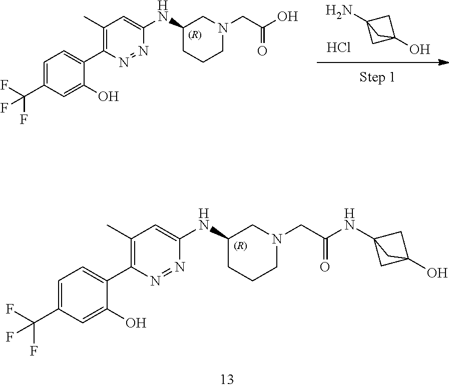

### Step 1: Synthesis of (R)-2-(3-((6-(2-hydroxy-4-(trifluoromethyl)phenyl)-5-methylpyridazin-3-yl)amino)piperidin-1-yl)-N-(3-hydroxybicyclo[1.1.1]pentan-1-yl)acetamide (13)

BOP (228 mg, 0.52 mmol, 1.2 eq.) was added into a colorless solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (177 mg, 0.43 mmol, 1.0 eq.), 3-aminobicyclo[1.1.1]pentan-1-ol hydrochloride (70 mg, 0.52 mmol, 2.2 mmol), and DIEA (299 μL, 222 mg, 1.72 mmol, 4.0 eq.) in DMF (3 mL) affording a slight yellow solution. The solution was stirred at rt for 1 h. The solution was subjected to preparative RP-HPLC purification using the following conditions: Column: Xselect CSH C18 OBD Column 30*150 mm 5 μm; Mobile Phase A: Water (0.1 vol-% FA), Mobile Phase B: ACN; Flow rate: 60 mL/min; Gradient: 5% B to 18% B in 7 min, 18% B to 18% B in 8 min, 18% B; Wavelength: 254 nm; RT1: 7.4 min. After lyophilization of the combined product containing fractions, the target compound 2-[(3R)-3-({6-[2-hydroxy-4-(trifluoromethyl)phenyl]-5-methylpyridazin-3-yl}amino)piperidin-1-yl]-N-{3-hydroxybicyclo[1.1.1]pentan-1-yl}acetamide was obtained as an off-white solid (10 mg, 4.7% yield). LC/MS: mass calcd for C₂₄H₂₈F₃N₅O₃: 491.21, found: m/z=492.25 [M+H]⁺. ¹H NMR (400 MHz, DMSO-d₆): δ 6.64-6.58 (m, 1H), 6.52-6.28 (m, 2H), 6.02 (s, 1H), 3.41 (s, 1H), 2.25-2.11 (m, 2H), 1.99-1.95 (m, 1H), 1.69 (s, 3H), 1.38-1.30 (m, 9H), 1.05 (s, 2H), 0.96-0.72 (m, 2H) ppm. ¹⁹F NMR (376 MHz, DMSO-d₆): δ −65.10 ppm.

### Example 14: 2-((R)-3-((6-(2-Hydroxy-4-(trifluoromethyl)phenyl)-5-methylpyridazin-3-yl)amino)piperidin-1-yl)-N-((1s,3S)-3-hydroxycyclobutyl)acetamide (14)

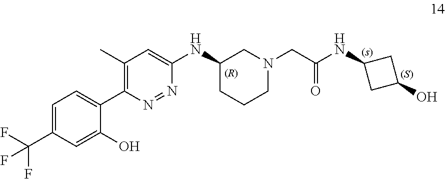

### Synthetic Scheme:

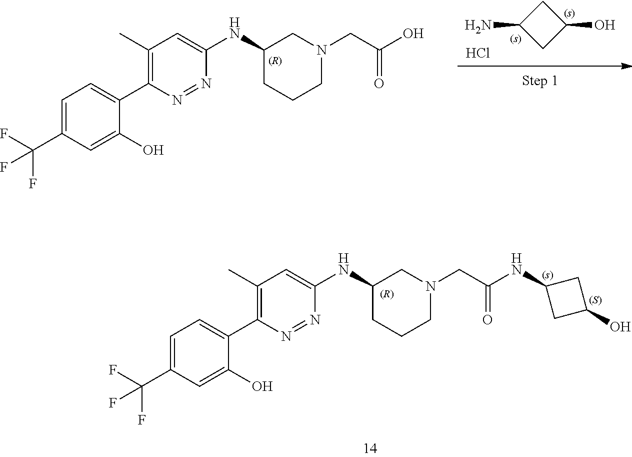

### Step 1: 2-((R)-3-((6-(2-hydroxy-4-(trifluoromethyl)phenyl)-5-methylpyridazin-3-yl)amino)piperidin-1-yl)-N-((1s,3S)-3-hydroxycyclobutyl)acetamide (14)

BOP (129 mg, 0.29 mmol, 1.2 eq.) was added into a colorless solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (100 mg, 0.24 mmol, 1.0 eq.), (1s,3s)-3-aminocyclobutan-1-ol hydrochloride (36 mg, 0.29 mmol, 1.2 eq.) and DIEA (168 μL, 125 mg, 0.98 mmol, 4.0 eq.) in DMF (3 mL) affording a slight yellow solution. The solution was stirred at rt for 1 h. The solution was submitted to preparative RP-HPLC using the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 26% B to 46% B in 7 min, 46% B; Wavelength: 254 nm; RT1: 5.87 min. After lyophilization of the combined product containing fractions, the target compound 2-[(3R)-3-({6-[2-hydroxy-4-(trifluoromethyl)phenyl]-5-methylpyridazin-3-yl}amino)piperidin-1-yl]-N-[(1s,3s)-3-hydroxycyclobutyl]acetamide (14) was obtained as an off-white solid (20 mg, 17.0% yield). LC/MS: mass calcd for C₂₃H₂₈F₃N₅O₃: 479.21, found: m/z=480.15 [M+H]⁺. ¹H NMR (300 MHz, Methanol-d₄) δ: 7.45-7.37 (m, 1H), 7.28-7.21 (m, 1H), 7.19-7.16 (m, 1H), 6.83 (s, 1H), 4.25-4.19 (m, 1H), 4.03-3.88 (m, 1H), 3.93-3.76 (m, 1H), 3.05-2.99 (m, 2H), 2.96-2.86 (m, 1H), 2.79-2.53 (m, 3H), 2.50-2.44 (m, 1H), 2.37-2.31 (m, 1H), 2.19-2.12 (m, 3H), 2.00-1.85 (m, 4H), 1.78-1.72 (m, 1H), 1.63-1.57 (m, 1H). ¹⁹F NMR (282 MHz, Methanol-d₄): δ −64.28 ppm.

### Example 15: 2-[(3R)-3-({6-[2-Hydroxy-4-(trifluoromethyl)phenyl]-5-methylpyridazin-3-yl}amino)piperidin-1-yl]-1-{2-hydroxy-7-azaspiro[3.5]nonan-7-yl}ethanone (15)

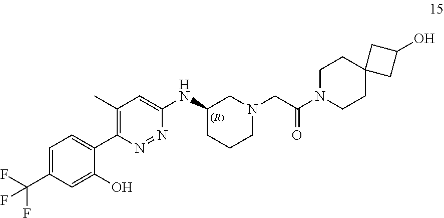

### Synthetic Scheme:

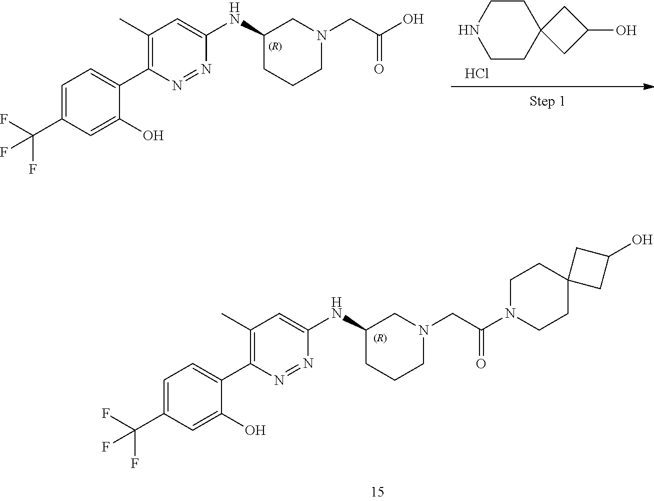

### Step 1: Synthesis of 2-[(3R)-3-({6-[2-hydroxy-4-(trifluoromethyl)phenyl]-5-methylpyridazin-3-yl}amino)piperidin-1-yl]-1-{2-hydroxy-7-azaspiro[3.5]nonan-7-yl}ethanone (15)

EDCI (91 mg, 0.48 mmol, 1.5 eq.) and HOBT (64 mg, 0.48 mmol, 1.5 eq.) were added into a yellow solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (130 mg, 0.32 mmol), 7-azaspiro[3.5]nonan-2-ol hydrochloride (113 mg, 0.63 mmol, 2.0 eq.) and DIEA (221 μL, 164 mg, 1.27 mmol, 4.0 eq.) in DMF (5 mL) affording a slight yellow solution. The solution was stirred at rt for 1 h. To this solution, EtOAc (10 mL) and water (10 mL) were added. After separation of phases, the aqueous phase was extracted with EtOAc (2×10 mL). The combined organic phases were washed with brine (3×30 mL), dried over Na₂SO₄, filtered, and the filtrate concentrated under reduced pressure using a rotary evaporator. The residue was subjected to preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wavelength: 254 nm; RT1: 6 min. After lyophilization, the target compound 2-[(3R)-3-({6-[2-hydroxy-4-(trifluoromethyl)phenyl]-5-methylpyridazin-3-yl}amino)piperidin-1-yl]-1-{2-hydroxy-7-azaspiro[3.5]nonan-7-yl}ethenone (15) (19.5 mg, 11.5% yield) was obtained as a light-yellow solid. LC/MS: mass calcd for C₂₇H₃₄F₃N₅O₃: 533.26, found: m/z=534.25 [M+H]⁺. ¹H NMR (400 MHz, Methanol-d₄): δ 7.45-7.39 (m, 1H), 7.28-7.21 (m, 1H), 7.21-7.16 (m, 1H), 6.83-6.78 (m, 1H), 4.32-4.20 (m, 1H), 4.20-4.07 (m, 1H), 3.59-3.44 (m, 4H), 3.27-3.13 (m, 2H), 2.91-2.86 (m, 1H), 2.59-2.54 (m, 1H), 2.43-2.23 (m, 4H), 2.16 (s, 3H), 1.91-1.83 (m, 2H), 1.76-1.63 (m, 5H), 1.59-1.49 (m, 3H) ppm. ¹⁹F NMR (376 MHz, Methanol-d₄): δ −64.28 ppm.

### Example 16: (R)-2-(3-((6-(2-Hydroxy-4-(trifluoromethyl)phenyl)-5-methylpyridazin-3-yl)amino)piperidin-1-yl)-1-(6-hydroxy-2-azaspiro[3.3]heptan-2-yl)ethan-1-one (16)

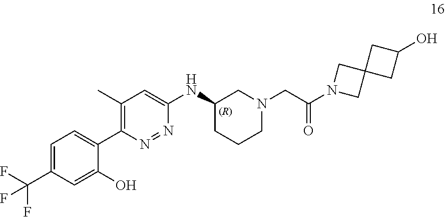

### Synthetic Scheme:

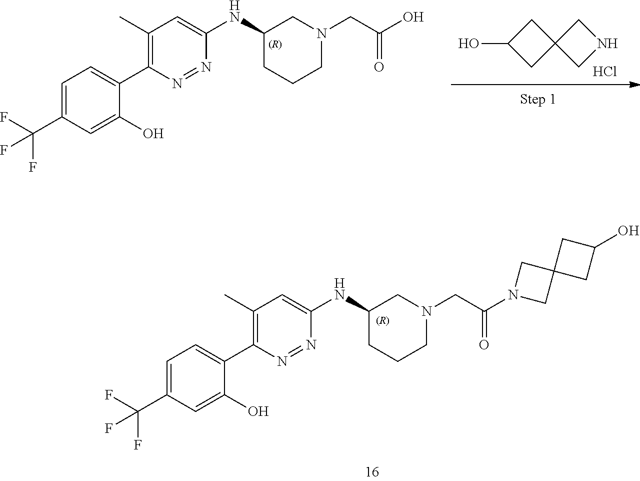

### Step 1: Synthesis of (R)-2-(3-((6-(2-hydroxy-4-(trifluoromethyl)phenyl)-5-methylpyridazin-3-yl)amino)piperidin-1-yl)-1-(6-hydroxy-2-azaspiro[3.3]heptan-2-yl)ethan-1-one (16)

A yellow solution of (R)-2-(3-((6-(2-hydroxy-4-(trifluoromethyl)phenyl)-5-methylpyridazin-3-yl)amino)piperidin-1-yl)acetic acid (prepared as disclosed in Example 10) (130 mg, 0.32 mmol), 2-azaspiro[3.3]heptan-6-ol hydrochloride (95 mg, 0.63 mmol, 2.0 eq.), EDCI (91 mg, 0.48 mmol, 1.5 eq.), HOBt (64 mg, 0.48 mmol, 1.5 eq.), and DIEA (276 μL, 205 mg, 1.59 mmol, 5.0 eq.) in DMF (3 mL) was stirred at rt for overnight. EtOAc (50 mL) and water (30 mL) were added giving a biphasic solution. After separation of phases, the aqueous phase was extracted with EtOAc (2×50 mL). The combined organic phases were washed with brine (1×30 mL), dried over Na₂SO₄, filtered, and the filtrate was concentrated under reduced pressure using a rotary evaporator. The crude product was purified by preparative RP-HPLC using the following conditions: Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 30% B to 60% B in 7 min, 60% B; Wavelength: 254 nm; RT1: 6 min. After lyophilization of the combined product containing fractions, the target compound (R)-2-(3-((6-(2-hydroxy-4-(trifluoromethyl)phenyl)-5-methylpyridazin-3-yl)amino)piperidin-1-yl)-1-(6-hydroxy-2-azaspiro[3.3]heptan-2-yl)ethan-1-one (16) (11.2 mg, 6.8% yield) was obtained as a colorless solid LC/MS: mass calcd for C₂₅H₃₀F₃N₅O₃: 505.23, found: m/z=506.25 [M+H]⁺. ¹H NMR (400 MHz, Methanol-d₄): δ 7.45-7.39 (m, 1H), 7.26-7.21 (m, 1H), 7.18 (s, 1H), 6.81 (s, 1H), 4.33-4.24 (m, 2H), 4.21-4.06 (m, 2H), 4.04-3.92 (m, 2H), 3.09-3.05 (m, 2H), 2.88-2.83 (m, 1H), 2.64-2.48 (m, 3H), 2.47-2.30 (m, 2H), 2.19-2.04 (m, 5H), 1.87-1.83 (m, 2H), 1.74-1.52 (m, 2H). ¹⁹F NMR (376 MHz, Methanol-d₄): δ −64.28 ppm.

### Example 17: (R)-2-(3-((6-(2-Hydroxy-4-(trifluoromethyl)phenyl)-5-methylpyridazin-3-yl)amino)piperidin-1-yl)-1-(7-hydroxy-2-azaspiro[3.5]nonan-2-yl)ethan-1-one (17)

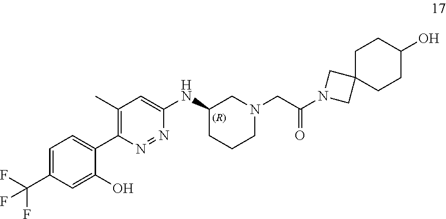

### Synthetic Scheme:

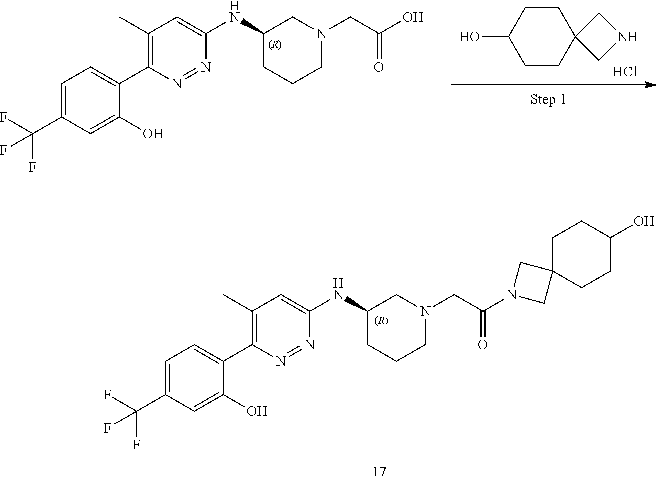

### Step 1: Synthesis of (R)-2-(3-((6-(2-Hydroxy-4-(trifluoromethyl)phenyl)-5-methylpyridazin-3-yl)amino)piperidin-1-yl)-1-(7-hydroxy-2-azaspiro[3.5]nonan-2-yl)ethan-1-one (17)

A light yellow solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (120 mg, 0.29 mmol, 1.0 eq.), 2-azaspiro[3.5]nonan-7-ol hydrochloride (62.3 mg, 0.35 mmol, 1.2 eq.), EDCI (84.1 mg, 0.44 mmol, 1.5 eq.), HOBt (59.3 mg, 0.44 mmol, 1.5 eq), DIEA (153 μL, 113.4 mg, 0.88 mmol, 3.0 eq.) in DMF (2 mL) was stirred at rt for 2 h. The reaction mixture was submitted to preparative RP-HPLC using the following conditions Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 24% B to 54% B in 7 min, 54% B; Wavelength: 254 nm; RT1: 5.72 min. After lyophilization of the combined product containing fractions, the target compound 1-{7-hydroxy-2-azaspiro[3.5]nonan-2-yl}-2-[(3R)-3-({6-[2-hydroxy-4-(trifluoromethyl)phenyl]-5-methylpyridazin-3-yl}amino)piperidin-1-yl]ethanone (17) (24.1 mg, 15.0%) was obtained as a light yellow solid. LC/MS: mass calcd for C₂₇H₃₄F₃N₅O₃: 533.26, found: m/z=534.20 [M+H]⁺. ¹H NMR (400 MHz, Methanol-d₄): δ 7.42 (d, J=7.9 Hz, 1H), 7.24 (d, J=8.0 Hz, 1H), 7.18 (s, 1H), 6.81 (s, 1H), 4.41-4.09 (m, 1H), 4.06-3.87 (m, 2H), 3.73-3.42 (m, 3H), 3.25-2.98 (m, 2H), 2.94-2.72 (m, 1H), 2.63-2.28 (m, 3H), 2.27-2.02 (m, 3H), 2.00-1.75 (m, 6H), 1.73-1.50 (m, 4H), 1.45-1.26 (m, 2H) ppm. ¹⁹F NMR (376 MHz, Methanol-d₄): δ −64.27 ppm.

### Example 18: 2-((R)-3-((6-(2-Hydroxy-4-(trifluoromethyl)phenyl)-5-methylpyridazin-3-yl)amino)piperidin-1-yl)-N-((1r,4R)-4-hydroxycyclohexyl)-N-methylacetamide (18)

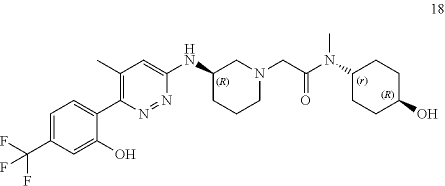

### Synthetic Scheme:

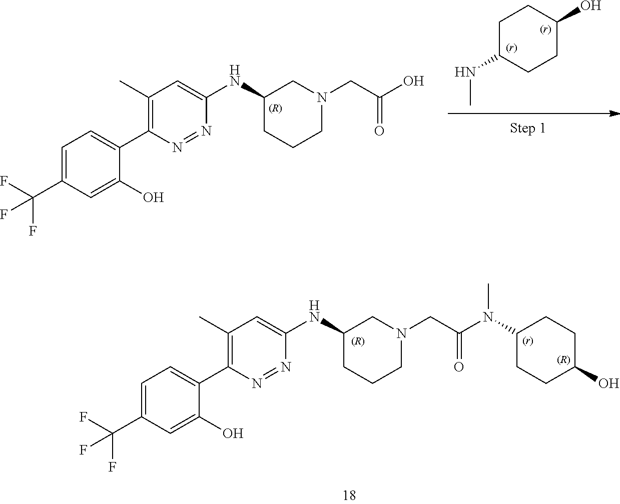

### Step 1: Synthesis of 2-((R)-3-((6-(2-Hydroxy-4-(trifluoromethyl)phenyl)-5-methylpyridazin-3-yl)amino)piperidin-1-yl)-N-((1r,4R)-4-hydroxycyclohexyl)-N-methylacetamide (18)

A light yellow solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (130 mg, 0.32 mmol), (1r,4r)-4-(methylamino)cyclohexan-1-ol (49 mg, 0.38 mmol, 1.2 eq.), EDCI (91 mg, 0.48 mmol, 1.5 eq.), HOBt (64 mg, 0.48 mmol, 1.5 eq.), DIEA (109 μL, 81 mg, 0.63 mmol) in DMF (2 mL) was stirred at rt for overnight. EtOAc (50 mL) and water (30 mL) were added giving a biphasic solution. After separation of phases, the aqueous phase was extracted with EtOAc (2×50 mL). The combined organic phases were washed with brine (1×30 mL), dried over Na₂SO₄, filtered, and the filtrate was concentrated under reduced pressure using a rotary evaporator. The crude product was purified by preparative RP-HPLC using the following conditions: Column: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 26% B to 56% B in 7 min, 56% B; Wavelength: 254 nm; RT1: 6 min. After lyophilization of combined product containing fractions, the target compound 2-[(3R)-3-({6-[2-hydroxy-4-(trifluoromethyl)phenyl]-5-methylpyridazin-3-yl}amino)piperidin-1-yl]-N-methyl-N-[(1r,4r)-4-hydroxycyclohexyl]acetamide (18) (10.1 mg, 5.9%) was obtained as a light yellow solid. LC/MS: mass calcd for C₂₆H₃₄F₃N₅O₃: 521.26, found: m/z=522.30 [M+H]⁺. ¹H NMR (400 MHz, DMSO-d₆): δ 7.47-7.31 (m, 1H), 7.27-7.13 (m, 2H), 6.75-6.67 (m, 1H), 4.29-3.76 (m, 2H), 3.41-3.24 (m, 1H), 3.21-3.07 (m, 2H), 3.05-2.73 (m, 3H), 2.65-2.58 (m, 2H), 2.31-1.92 (m, 6H), 1.91-1.82 (m, 2H), 1.80-1.72 (m, 1H), 1.67-1.56 (m, 2H), 1.55-1.38 (m, 3H), 1.32-1.18 (m, 3H) ppm. ¹⁹F NMR (376 MHz, DMSO-d₆): δ −64.31 ppm.

### Example 19:1-{2-[(3R)-3-({4-[2-Hydroxy-4-(trifluoromethyl)phenyl]-5H,6H,7H-cyclopenta[d]pyridazin-1-yl}amino)piperidin-1-yl]ethyl}piperidin-4-ol (19)

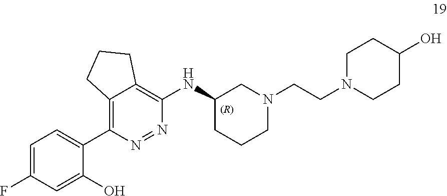

### Synthetic Scheme

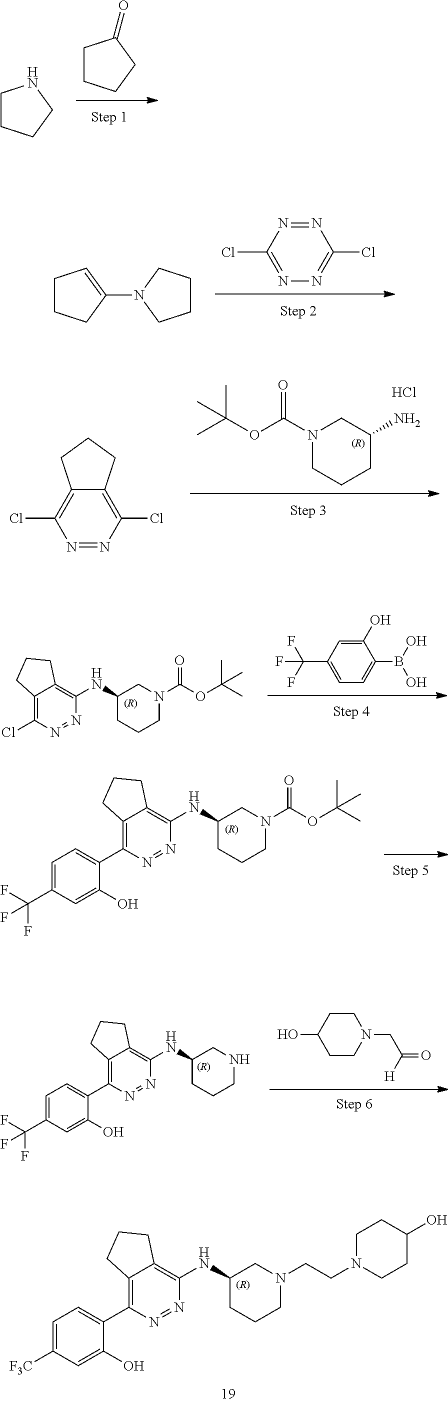

### Step 1: Synthesis of 1-(cyclopent-1-en-1-yl)pyrrolidine

To a light yellow solution of pyrrolidine (10.00 g, 140.6 mmol, 1.0 eq.) in toluene (50 mL) were added cyclopentanone (11.83 g, 140.6 mmol, 1 eq.) and TsOH (0.10 g, 0.56 mmol, 0.004 eq.). After stirring of the reaction mixture for 16 h at 130° C. (oil bath), the reaction mixture was cooled to rt, and the residue was concentrated under reduced pressure using a rotary evaporator to yield 1-(cyclopent-1-en-1-yl)pyrrolidine (11.0 g, 57.0% yield) as a brown oil. The residue was used directly in the next step without further isolation and characterization.

### Step 2: Synthesis of 1,4-dichloro-5H,6H, 7H-cyclopenta[d]pyridazine

To a red solution of dichloro-1,2,4,5-tetrazine (4.40 g, 29.15 mmol, 0.5 eq) in DCM (150 mL) was added 1-(cyclopent-1-en-1-yl)pyrrolidine (8.00 g, 58.3 mmol, 1 eq.) at 0° C. After stirring of the reaction mixture for 15 min at 0° C., the reaction mixture was concentrated at 0° C. under reduced pressure using a rotary evaporator. The residue was purified by silica gel column chromatography using (0~45% diethyl ether/PE) gradients to afford 1,4-dichloro-5H,6H,7H-cyclopenta[d]pyridazine (2.6 g, 23.6% yield) as light yellow solid. LC/MS: mass calcd for C₇H₆Cl₂N₂: 187.99, found: m/z=189.10 [M+H]⁺. ¹H NMR (400 MHz, DSMO-d₆): δ 3.06-3.10 (m, 4H), 2.11-2.19 (m, 2H) ppm.

### Step 3: Synthesis of tert-butyl (3R)-3-({4-chloro-5H,6H,7H-cyclopenta[d]pyridazin-1-yl}amino)piperidine-1-carboxylate

To a light yellow solution of 1,4-dichloro-5H,6H,7H-cyclopenta[d]pyridazine (900 mg, 4.76 mmol, 1 eq.) in NMP (10 mL) were added tert-butyl (3R)-3-aminopiperidine-1-carboxylate (1.14 g, 5.71 mmol, 1.2 eq.), and DIEA (1.66 mL, 1.23 g, 9.52 mmol, 2 eq.). After stirring for 12 h at 150° C. in a sealed tube, the reaction mixture was cooled to rt, and the residue was extracted with EtOAc (3×50 mL). The combined organic phases were washed with water (3×50 mL), dried over Na₂SO₄, filtered, and the filtrate was concentrated under reduced pressure using a rotary evaporator. The residue was purified by silica gel column chromatography with (0~45% EA/PE) gradients to yield tert-butyl (3R)-3-({4-chloro-5H,6H,7H-cyclopenta[d]pyridazin-1-yl}amino)piperidine-1-carboxylate (0.55 g, 32.7% yield) as light yellow solid. LC/MS: mass calcd for C₁₇H₂₅ClN₄O₂: 352.17, found: m/z=353.20 [M+H]⁺. ¹H NMR (400 MHz, DMSO-d₆): δ 6.27 (s, 1H), 3.30-4.05 (m, 3H), 2.49-3.28 (m, 6H), 2.01-2.13 (m, 2H), 1.89-1.94 (m, 1H), 1.72 (s, 1H), 1.53 (s, 1H), 1.23-1.46 (m, 10H) ppm.

### Step 4: Synthesis of tert-butyl (3R)-3-({4-[2-hydroxy-4-(trifluoromethyl)phenyl]-5H,6H,7H-cyclopenta[d]pyridazin-1-yl}amino)piperidine-1-carboxylate

To a solution of tert-butyl (3R)-3-({4-chloro-5H,6H,7H-cyclopenta[d]pyridazin-1-yl}amino)piperidine-1-carboxylate (250 mg, 0.701 mmol, 1.0 eq.) and 2-hydroxy-4-(trifluoromethyl)phenylboronic acid (175.1 mg, 0.85 mmol, 1.2 eq.) in 3.75 mL of a mixture of 1,4-dioxane and water (4:1, v/v) were added Pd(PPh₃)₄ (40.9 mg, 0.035 mmol, 0.05 eq.) and NaHCO₃ (119.0 mg, 1.42 mmol, 2.0 eq.). After stirring of the reaction mixture for 5 h at 100° C. in a sealed tube under an atmosphere of N₂, the resulting reaction mixture was concentrated under reduced pressure using a rotary evaporator. The residue was purified by silica gel column chromatography with (0~50% EA/PE) gradients to yield tert-butyl (3R)-3-({4-[2-hydroxy-4-(trifluoromethyl)phenyl]-5H,6H,7H-cyclopenta[d]pyridazin-1-yl}amino)piperidine-1-carboxylate (246 mg, 72.6% yield) as light yellow solid. LC/MS: mass calcd for C₂₄H₂₉F₃N₄O₃: 478.22, found: m/z=479.25 [M+H]⁺. ¹H NMR (400 MHz, DMSO-d₆): δ 6.74-6.76 (m, 1H), 7.21-7.24 (m, 2H), 6.47 (s, 1H), 3.85-4.22 (m, 3H), 3.65-3.82 (m, 1H), 3.03-3.28 (m, 2H), 2.68-2.98 (m, 4H), 2.01-2.14 (m, 2H), 1.90-1.96 (m, 1H), 1.75 (s, 1H), 1.57 (s, 1H), 1.23-1.46 (m, 10H) ppm. ¹⁹F NMR (376 MHz, DMSO-d₆): δ −61.35 ppm.

### Step 5: Synthesis of 2-{4-[(3R)-piperidin-3-ylamino]-5H,6H,7H-cyclopenta[d]pyridazin-1-yl}-5-(trifluoromethyl)phenol

To a light yellow solution of tert-butyl (3R)-3-({4-[2-hydroxy-4-(trifluoromethyl)phenyl]-5H,6H,7H-cyclopenta[d]pyridazin-1-yl}amino)piperidine-1-carboxylate (200 mg, 0.42 mmol, 1 eq.) in DCM (5 mL) was added TFA (1 mL). After stirring for 30 min at rt, the suspension was concentrated under reduced pressure using a rotary evaporator. The residue was purified by reverse flash chromatography using the following conditions: column, C18 silica gel; mobile phase, ACN in water, 10% to 60% gradient in 20 min; detector, UV 254 nm to afford 2-{4-[(3R)-piperidin-3-ylamino]-5H,6H,7H-cyclopenta[d]pyridazin-1-yl}-5-(trifluoromethyl)phenol (71 mg, 44.9% yield) as light yellow solid. LC/MS: mass calcd for C₁₉H₂₁F₃N₄O: 378.17, found: m/z=379.20 [M+H]⁺.

### Step 6: Synthesis of 1-{2-[(3R)-3-({4-[2-hydroxy-4-(trifluoromethyl)phenyl]-5H,6H,7H-cyclopenta[d]pyridazin-1-yl}amino)piperidin-1-yl]ethyl}piperidin-4-ol (19)

To a light yellow solution of 2-{4-[(3R)-piperidin-3-ylamino]-5H,6H,7H-cyclopenta[d]pyridazin-1-yl}-5-(trifluoromethyl)phenol (100 mg, 0.26 mmol, 1 eq.) in MeOH (2 mL) were added 2-(4-hydroxypiperidin-1-yl)acetaldehyde (45.4 mg, 0.32 mmol, 1.2 eq.) (prepared according to the procedure described for Example 21), and NaBH₃CN (49.8 mg, 0.79 mmol, 3.0 eq.). After stirring for 2 h at rt, the suspension was concentrated under reduced pressure using a rotary evaporator. The residue was purified by reverse flash chromatography using the following conditions: Column: Xselect CSH C18 OBD Column 30*150 mm 5 μm; Mobile Phase A: Water (0.1% FA), Mobile Phase B: ACN; Flow rate: 60 mL/min; Gradient: 5% B to 30% B in 7 min, 30% B; Wavelength: 254 nm; RT1: 5.87 min, to yield the target compound 1-{2-[(3R)-3-({4-[2-hydroxy-4-(trifluoromethyl)phenyl]-5H,6H,7H-cyclopenta[d]pyridazin-1-yl}amino)piperidin-1-yl]ethyl}piperidin-4-ol (19) (15.3 mg, 11.4% yield) as light yellow solid after lyophilization of the combined product containing fractions. LC/MS: MS (ESI) calcd for C₂₆H₃₄F₃N₅O₂: 505.27, found: m/z=506.30 [M+H]⁺. ¹H NMR (300 MHz, Methanol-d₄): δ 8.48-8.36 (s, 1H), 7.74-7.71 (m, 1H), 7.21-7.18 (m, 2H), 4.43-4.11 (m, 1H), 3.94-3.89 (m, 1H), 3.49-3.33 (m, 3H), 3.24-3.08 (m, 6H), 3.03-2.94 (m, 1H), 2.92-2.84 (m, 3H), 2.75-2.67 (m, 1H), 2.32-2.17 (m, 3H), 2.15-1.91 (m, 4H), 1.89-1.70 (m, 4H), 1.63-1.54 (m, 1H) ppm. ¹⁹F NMR (282 MHz, Methanol-d₄): δ −64.45 ppm.

### Example 20: 2-[(3R)-3-{[4-(4-Fluoro-2-hydroxyphenyl)-5H,6H,7H-cyclopenta[d]pyridazin-1-yl]amino}piperidin-1-yl]-1-(4-hydroxypiperidin-1-yl) ethenone (20)

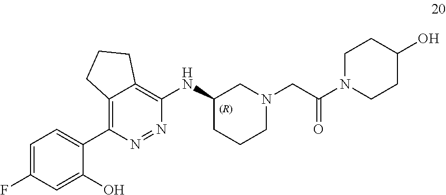

### Synthetic Scheme:

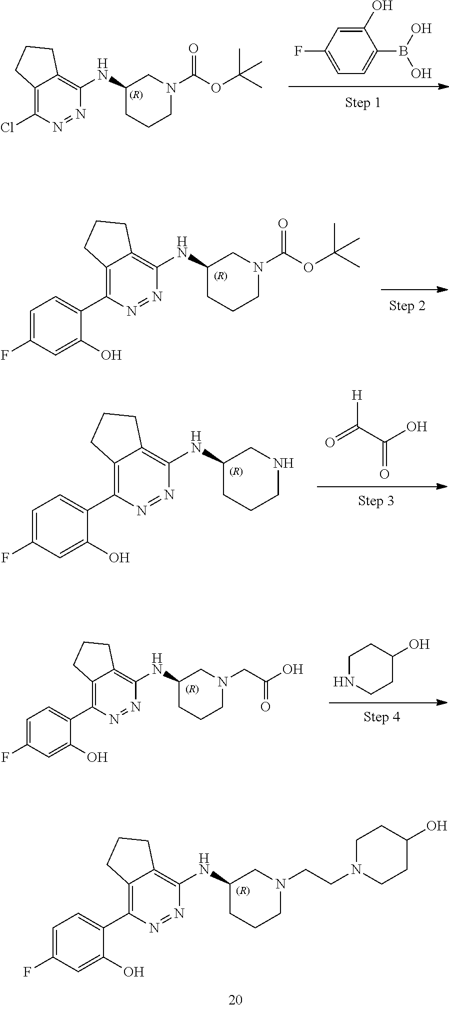

### Step 1: Synthesis of tert-butyl (3R)-3-{[4-(4-fluoro-2-hydroxyphenyl)-5H,6H, 7H-cyclopenta[d]pyridazin-1-yl]amino}piperidine-1-carboxylate

To a solution of tert-butyl (3R)-3-({4-chloro-5H,6H,7H-cyclopenta[d]pyridazin-1-yl}amino)piperidine-1-carboxylate (250 mg, 0.71 mmol, 1 eq.) and 4-fluoro-2-hydroxyphenylboronic acid (132.6 mg, 0.85 mmol, 1.2 eq.) in in a 3.75 mL of a mixture of 1,4-dioxane and water (4:1, v/v) were added Pd(PPh₃)₄ (40.9 mg, 0.035 mmol, 0.05 eq.) and NaHCO₃ (119.0 mg, 1.42 mmol, 2.0 eq.). After stirring the reaction mixture for 5 h at 100° C. under an atmosphere of N₂ in a sealed tube, the resulting reaction mixture was concentrated under reduced pressure using a rotary evaporator. The residue was purified by silica gel column chromatography (0~50% EA/PE) gradients giving tert-butyl (3R)-3-{[4-(4-fluoro-2-hydroxyphenyl)-5H,6H,7H-cyclopenta[d]pyridazin-1-yl]amino}piperidine-1-carboxylate (250 mg, 82.35% yield) as light yellow solid after evaporation of the combined product containing fractions under reduced pressure using a rotary evaporator. LC/MS: mass calcd for C₂₃H₂₉FN₄O₃: 428.22, found: m/z=429.25 [M+H]⁺. ¹H NMR (400 MHz, DMSO-d₆): δ 7.63-7.60 (m, 1H), 6.74-6.70 (m, 2H), 6.38 (s, 1H), 4.05-3.82 (m, 2H), 3.80-3.65 (m, 1H), 3.28-2.86 (m, 3H), 2.83-2.74 (m, 3H), 2.12-2.02 (m, 3H), 1.95-1.90 (m, 1H), 1.74 (s, 1H), 1.57 (s, 1H), 1.47-1.23 (m, 10H) ppm. ¹⁹F NMR (376 MHz, DMSO-d₆): δ −111.38 ppm.

### Step 2: Synthesis of 5-fluoro-2-{4-[(3R)-piperidin-3-ylamino]-5H,6H,7H-cyclopenta[d]pyridazin-1-yl}phenol

To a light yellow solution of tert-butyl (3R)-3-{[4-(4-fluoro-2-hydroxyphenyl)-5H,6H,7H-cyclopenta[d]pyridazin-1-yl]amino}piperidine-1-carboxylate (250 mg, 0.58 mmol) in DCM (5 mL) was added TFA (1 mL). After stirring the reaction mixture for 30 min at rt, the suspension was concentrated under reduced pressure using a rotary evaporator. The residue was purified by reverse flash chromatography with the following conditions: column, C18 silica gel; mobile phase, ACN in water, 10% to 60% gradient in 20 min; detector, UV 254 nm to yield 5-fluoro-2-{4-[(3R)-piperidin-3-ylamino]-5H,6H,7H-cyclopenta[d]pyridazin-1-yl}phenol (180 mg, 94.0% yield) as light yellow solid after lyphilization of the combined product containing fractions. LC/MS: mass calcd for C₁₈H₂₁FN₄O: 328.17, found: m/z=329.15 [M+H]⁺.

### Step 3: Synthesis of [(3R)-3-{[4-(4-fluoro-2-hydroxyphenyl)-5H,6H, 7H-cyclopenta[d]pyridazin-1-yl]amino}piperidin-1-yl]acetic Acid

To a light yellow solution of 5-fluoro-2-{4-[(3R)-piperidin-3-ylamino]-5H,6H,7H-cyclopenta[d]pyridazin-1-yl}phenol (90 mg, 0.27 mmol, 1 eq.) in MeOH (2 mL) were added glyoxylate (24.4 mg, 0.33 mmol, 1.2 eq.) and NaBH₃CN (51.7 mg, 0.82 mmol, 3.0 eq.). After stirring for 2 h at rt, the suspension was concentrated under reduced pressure using a rotary evaporator. The residue was purified by reverse flash chromatography with the following conditions: column, C18 silica gel; mobile phase, ACN in water, 10% to 50% gradient in 20 min; detector, UV 254 nm to yield [(3R)-3-{[4-(4-fluoro-2-hydroxyphenyl)-5H,6H,7H-cyclopenta[d]pyridazin-1-yl]amino}piperidin-1-yl]acetic acid (100 mg, 94.4% yield) as light yellow solid after lyophilization of the combined product containing fractions. LC/MS: MS (ESI) calcd for C₂₀H₂₃FN₄O₃: 386.18, found: m/z=387.15 [M+H]⁺.

### Step 4: Synthesis of 2-[(3R)-3-{[4-(4-fluoro-2-hydroxyphenyl)-5H,6H, 7H-cyclopenta[d]pyridazin-1-yl]amino}piperidin-1-yl]-1-(4-hydroxypiperidin-1-yl)ethanone (20)

To a light yellow solution of [(3R)-3-{[4-(4-fluoro-2-hydroxyphenyl)-5H,6H,7H-cyclopenta[d]pyridazin-1-yl]amino}piperidin-1-yl]acetic acid (90 mg, 0.23 mmol, 1 eq.) in DMF (2 mL), piperidin-4-ol (28.3 mg, 0.28 mmol, 1.2 eq.), EDCI (89.3 mg, 0.47 mmol, 2.0 eq.), and HOBt (62.9 mg, 0.47 mmol, 2.0 eq.) were added under stirring at rt. The suspension was stirred for 4 h at rt resulting in a brown suspension. The reaction was quenched by the addition of a saturated aqueous NH₄Cl solution (8 mL) at 0° C. The resulting mixture was extracted with EtOAc (3×20 mL). The combined organic layers were washed with water (3×20 mL) and dried over anhydrous Na₂SO₄. After filtration, the filtrate was concentrated under reduced pressure using a rotary evaporator. The residue was purified by reverse flash chromatography using the following condition: Column: XBridge Prep Phenyl OBD Column, 19*100 mm, 5 μm; Mobile Phase A: Water (10 mmol/L NH₄HCO₃), Mobile Phase B: ACN; Flow rate: 60 mL/min; Gradient: 5% B to 95% B in 7 min, 95% B; Wavelength: 254 nm; RT1: 6 min, to afford the target compound 2-[(3R)-3-{[4-(4-fluoro-2-hydroxyphenyl)-5H,6H,7H-cyclopenta[d]pyridazin-1-yl]amino}piperidin-1-yl]-1-(4-hydroxypiperidin-1-yl)ethanone (20) (22.6 mg, 20.6% yield) as a light yellow solid after lyophilization of the combined product containing fractions. LC/MS: MS (ESI) calcd for C₂₅H₃₂FN₅O₃: 469.25, found: m/z=470.25 [M+H]⁺. ¹H NMR (400 MHz, Methanol-d₄): δ 7.61-7.56 (m, 1H), 6.67-6.63 (m, 2H), 4.36-4.25 (m, 1H), 4.19-4.02 (m, 1H), 3.98-3.78 (m, 2H), 3.43-3.39 (m, 1H), 3.41-3.12 (m, 4H), 3.11-2.93 (m, 2H), 2.90-2.88 (m, 2H), 2.75-2.56 (m, 1H), 2.38-2.11 (m, 4H), 2.08-1.88 (m, 4H), 1.78-1.53 (m, 3H), 1.50-1.37 (m, 1H) ppm. ¹⁹F NMR (376 MHz, Methanol-d₄): δ −133.28 ppm.

### Example 21:1-{2-[(3R)-3-{[4-(4-Fluoro-2-hydroxyphenyl)-5H,7H,6H,7H-cyclopenta[d]pyridazin-1-yl]amino}piperidin-1-yl]ethyl}piperidin-4-ol (21)

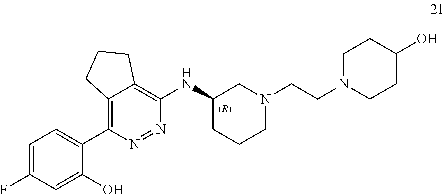

### Synthetic Scheme:

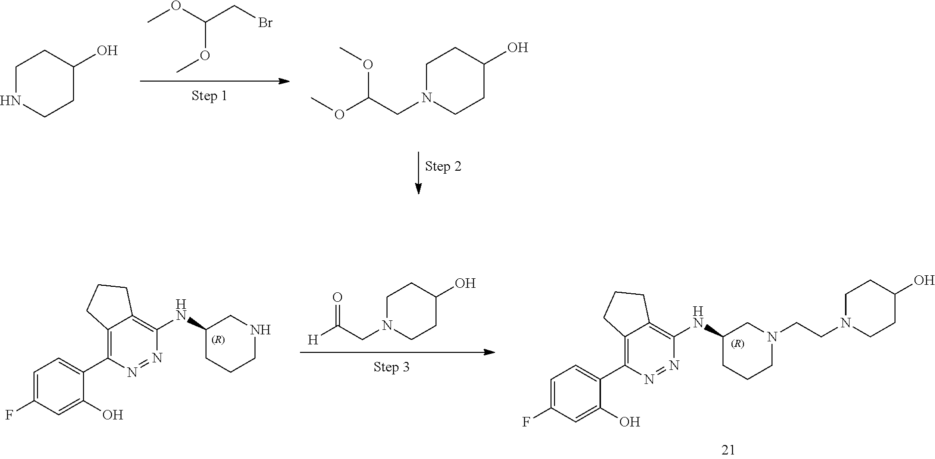

### Step 1: Synthesis of 1-(2,2-dimethoxyethyl)piperidin-4-ol

A colorless suspension of piperidin-4-ol (2.00 g, 19.77 mmol), 2-bromo-1,1-dimethoxyethane (3.34 g, 19.77 mmol, 1.0 eq.), and K₂CO₃ (5.47 g, 39.55 mmol, 2.0 eq.) in 1,4-dioxane (20 mL) was stirred at 100° C. overnight. After cooling to rt, EtOAc (60 mL) and water (60 mL) were added. After separation of phases, the aqueous phase was extracted with EtOAc (2×50 mL). The combined organic phases were washed with brine (2×50 mL), dried over Na₂SO₄, filtered, and the filtrate was concentrated under reduced pressure using a rotary evaporator to afford 1-(2,2-dimethoxyethyl)piperidin-4-ol as an off-white solid (1.8 g, 42.3% yield) which was used without further isolation and characterization in the next step. LC/MS: mass calcd for C₉H₁₉NO₃: 189.14, found: m/z=190.20=[M+H]⁺.

### Step 2: Synthesis of 2-(4-hydroxypiperidin-1-yl)acetaldehyde

1-(2,2-Dimethoxyethyl)piperidin-4-ol (1.8 g, 9.51 mmol) in HCl (20 mL, 6 M in water) was stirred at 100° C. overnight. The reaction was concentrated under reduced pressure to afford 2-(4-hydroxypiperidin-1-yl)acetaldehyde as a colorless oil (900 mg, 66.2% yield). The product was used directly in the next step without any further isolation and characterization. LCMS: mass calcd for C₇H₁₃NO₂: 143.09, found: m/z=144.15 [M+H]₊.

### Step 3: Synthesis of 1-{2-[(3R)-3-{[4-(4-Fluoro-2-hydroxyphenyl)-5H,6H, 7H-cyclopenta[d]pyridazin-1-yl]amino}piperidin-1-yl]ethyl}piperidin-4-ol (21)

To a light yellow solution of 5-fluoro-2-{4-[(3R)-piperidin-3-ylamino]-5H,6H,7H-cyclopenta[d]pyridazin-1-yl}phenol (100 mg, 0.31 mmol, 1.0 eq.) in MeOH (2 mL) were added 2-(4-hydroxypiperidin-1-yl)acetaldehyde (52.3 mg, 0.37 mmol, 1.2 eq.) and NaBH₃CN (57.4 mg, 0.92 mmol, 3.0 eq.) to afford a light yellow solution. After stirring for 2 h at rt, the suspension was concentrated under reduced pressure. The residue was purified by reverse flash chromatography with the following conditions: Column: Xselect CSH C18 OBD Column 30*150 mm 5 μm; Mobile Phase A: Water (0.1 vol-% FA), Mobile Phase B: ACN; Flow rate: 60 mL/min; Gradient: 3% B to 20% B in 7 min, 20% B; Wavelength: 254 nm; RT1: 5.87 min; to yield the target compound 1-{2-[(3R)-3-{[4-(4-fluoro-2-hydroxyphenyl)-5H,6H,7H-cyclopenta[d]pyridazin-1-yl]amino}piperidin-1-yl]ethyl}piperidin-4-ol (21) (24.6 mg, 17.6% yield) as light yellow solid after lyophilization of the combined product containing fractions. LC/MS: MS (ESI) calcd for C₂₅H₃₄FN₅O₂: 455.27, found: m/z=456.25 [M+H]⁺. ¹H NMR (300 MHz, DMSO-d₆): δ 8.12 (s, 1H), 7.57-7.47 (m, 1H), 6.69-6.64 (m, 2H), 4.22-4.19 (m, 1H), 3.43-3.38 (m, 2H), 3.08-2.94 (m, 4H), 3.03-2.94 (m, 2H), 2.83-2.60 (m, 7H), 2.21-1.98 (m, 4H), 1.97-1.67 (m, 4H), 1.51-1.35 (m, 4H) ppm. ¹⁹F NMR (282 MHz, DMSO-d₆): δ −111.39 ppm.

### Example 22: (R)-2-(3-((4-(2-Hydroxy-4-(trifluoromethyl)phenyl)-5,7-dihydrofuro[3,4-d]pyridazin-1-yl)amino)piperidin-1-yl)-1-(3-hydroxyazetidin-1-yl)ethan-1-one (22)

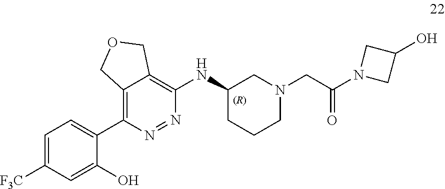

### Synthetic Scheme:

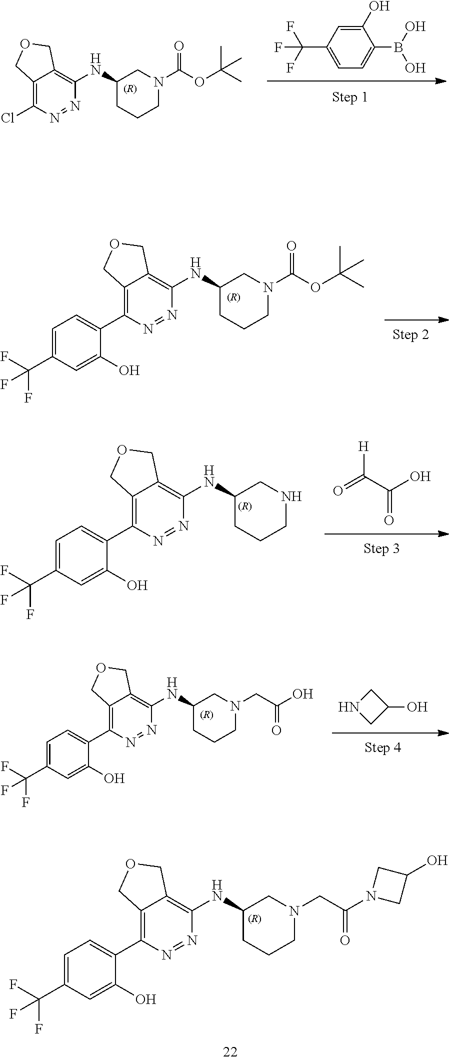

### Step 1: Synthesis of tert-butyl (3R)-3-({4-[2-hydroxy-4-(trifluoromethyl)phenyl]-5H,7H-furo[3,4-d]pyridazin-1-yl}amino)piperidine-1-carboxylate

To a solution of tert-butyl (3R)-3-({4-chloro-5H,7H-furo[3,4-d]pyridazin-1-yl}amino)piperidine-1-carboxylate (500 mg, 1.40 mmol, 1 eq.) and 2-hydroxy-4-(trifluoromethyl)phenylboronic acid (348.2 mg, 1.69 mmol, 1.2 eq.) in 5 mL of a mixture of 1,4-dioxane and water (4:1, v/v) were added Pd(PPh₃)₄ (81.4 mg, 0.070 mmol, 0.05 eq.) and NaHCO₃ (355.1 mg, 4.23 mmol, 3.0 eq.). After stirring the reaction mixture in a sealed tube for 5 h at 100° C. under an atmosphere of N₂ and cooling to rt, the resulting mixture was concentrated under reduced pressure using a rotary evaporator. The residue was purified by silica gel column chromatography (0~50% EA/PE) gradients to yield tert-butyl (3R)-3-({4-[2-hydroxy-4-(trifluoromethyl)phenyl]-5H,7H-furo[3,4-d]pyridazin-1-yl}amino)piperidine-1-carboxylate (373 mg, 55.1% yield) as light yellow oil after concentrating the combined product containing fractions under reduce pressure using a rotary evaporator. LC/MS: MS (ESI) calcd for C₂₃H₂₇F₃N₄O₄: 480.20, found: m/z=481.25 [M+H]⁺. ¹⁹F NMR (376 MHz, DMSO-d₆): δ −61.43 ppm.

### Step 2: Synthesis of 2-{4-[(3R)-piperidin-3-ylamino]-5H, 7H-furo[3,4-d]pyridazin-1-yl}-5-(trifluoromethyl)phenol

To a light yellow solution of tert-butyl (3R)-3-({4-[2-hydroxy-4-(trifluoromethyl)phenyl]-5H,7H-furo[3,4-d]pyridazin-1-yl}amino)piperidine-1-carboxylate (330 mg, 0.69 mmol, 1 eq.) in dioxane (1 mL) was added a solution of HCl (3 mL, 12 mmol, 4 M in 1,4-dioxane). After stirring the reaction mixture for 30 min at rt, the suspension was concentrated under reduced pressure using a rotary evaporator. The residue was purified by reverse flash chromatography using the following conditions: column, C18 silica gel; mobile phase, ACN in water, 10% to 60% gradient in 20 min; detector, UV 254 nm to afford 2-{4-[(3R)-piperidin-3-ylamino]-5H,7H-furo[3,4-d]pyridazin-1-yl}-5-(trifluoromethyl)phenol (170 mg, 65.1% yield) as light yellow solid after lyophilization of the combined product containing fractions. LC/MS: MS (ESI) calcd for C₁₈H₁₉F₃N₄O₂: 380.15, found: m/z=381.20 [M+H]⁺.

### Step 3: Synthesis of [(3R)-3-({4-[2-hydroxy-4-(trifluoromethyl)phenyl]-5H, 7H-furo[3,4-d]pyridazin-1-yl}amino)piperidin-1-yl]acetic Acid

To a light yellow solution of 2-{4-[(3R)-piperidin-3-ylamino]-5H,7H-furo[3,4-d]pyridazin-1-yl}-5-(trifluoromethyl)phenol (85 mg, 0.22 mmol, 1.0 eq.) in MeOH (2 mL) were added glyoxylate (33.1 mg, 0.45 mmol, 2.0 eq.), and NaBH₃CN (42.1 mg, 0.67 mmol, 3.0 eq.). After stirring for 2 h at rt, the suspension was concentrated under reduced pressure using a rotary evaporator. The residue was purified by reverse flash chromatography using the following conditions: column, C18 silica gel; mobile phase, ACN in water, 10% to 50% gradient in 20 min; detector, UV 254 nm to afford [(3R)-3-({4-[2-hydroxy-4-(trifluoromethyl)phenyl]-5H,7H-furo[3,4-d]pyridazin-1-yl}amino)piperidin-1-yl]acetic acid (80 mg, 81.7% yield) as light yellow solid. LC/MS: MS (ESI) calcd for C₂₀H₂₁F₃N₄O₄: 438.15, found: m/z=439.20 [M+H]⁺.

### Step 4: Synthesis of 2-[(3R)-3-({4-[2-hydroxy-4-(trifluoromethyl)phenyl]-5H, 7H-furo[3,4-d]pyridazin-1-yl}amino)piperidin-1-yl]-1-(3-hydroxyazetidin-1-yl)ethanone (22)

To a light yellow solution of [(3R)-3-({4-[2-hydroxy-4-(trifluoromethyl)phenyl]-5H,7H-furo[3,4-d]pyridazin-1-yl}amino)piperidin-1-yl]acetic acid (100 mg, 0.23 mmol, 1 eq.) in DMF (2 mL), azetidin-3-ol (not HCl salt?) (25.0 mg, 0.34 mmol, 1.5 eq.), EDCI (87.5 mg, 0.46 mmol, 2 eq.), DIEA (119 μL, 88.4 mg, 0.68 mmol, 3.0 eq.) and HOBT (61.6 mg, 0.46 mmol, 2.0 eq.) were added under stirring at rt. The suspension was stirred for 4 h at rt to result in a brown suspension. The reaction was quenched by the addition of a saturated aqueous NH₄Cl solution (8 mL) at 0° C. The resulting mixture was extracted with EtOAc (3×20 mL). The combined organic layers were washed with water (3×20 mL) and dried over anhydrous Na₂SO₄. After filtration, the filtrate was concentrated under reduced pressure using a rotary evaporator. The residue was purified using the following condition: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 33% B to 63% B in 7 min, 63% B; Wavelength: 254 nm; RT1: 6 min to yield the target compound 2-[(3R)-3-({4-[2-hydroxy-4-(trifluoromethyl)phenyl]-5H,7H-furo[3,4-d]pyridazin-1-yl}amino)piperidin-1-yl]-1-(3-hydroxyazetidin-1-yl)ethanone (16.2 mg, 13.9% yield) as a light yellow solid after lyophilization of the combined product containing fractions. LC/MS: MS (ESI) calcd for C₂₃H₂₆F₃N₅O₄: 493.20, found: m/z=494.20 [M+H]⁺. ¹H NMR (400 MHz, Methanol-d₄): δ 7.41-7.39 (m, 1H), 7.20-7.19 (m, 2H), 5.37 (s, 2H), 5.06 (s, 2H), 4.67-4.49 (m, 2H), 4.41-4.35 (m, 1H), 4.27-4.19 (m, 1H), 4.15-4.05 (m, 1H), 3.87-3.73 (m, 1H), 3.19-3.03 (m, 2H), 2.97-2.85 (m, 1H), 2.62-2.50 (m, 1H), 2.45-2.28 (m, 2H), 1.93-1.77 (m, 2H), 1.71-1.52 (m, 2H) ppm. ¹⁹F NMR (376 MHz, Methanol-d₄): δ−64.53 ppm.

### Example 23: (R)-2-(3-((4-(2-Hydroxy-4-(trifluoromethyl)phenyl)-5,7-dihydrofuro[3,4-d]pyridazin-1-yl)amino)piperidin-1-yl)-1-(4-hydroxypiperidin-1-yl)ethan-1-one (23)

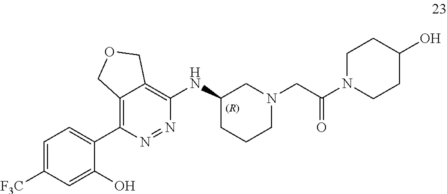

### Synthetic Scheme:

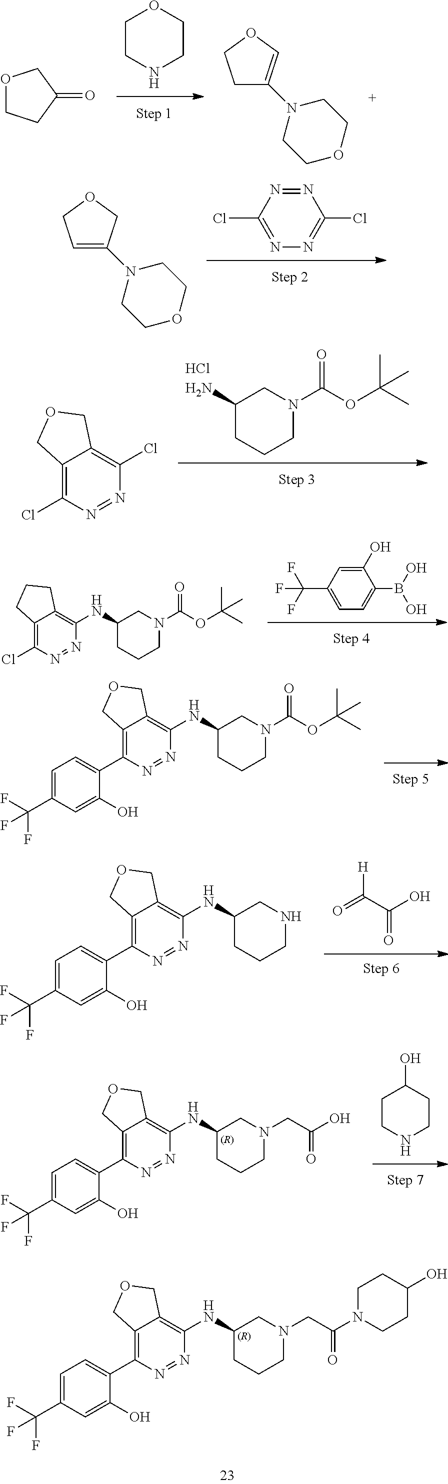

### Step 1: Synthesis of 4-(2,5-dihydrofuran-3-yl)morpholine

To a 200 mL sealed tube equipped with a stirring bar were added dihydrofuran-3-one (6.91 g, 80.3 mmol, 1 eq.), morpholine (4.70 mL, 54.0 mmol, 2.0 eq.), MgSO₄ (30.0 g, 249 mmol, 3.1 eq.) in Et₂O (150 mL). The resulting reaction mixture was stirred at rt for 48 h. The reaction mixture was filtered, and the filtrate was concentrated under reduced pressure using a rotary evaporator to yield 4-(2,5-dihydrofuran-3-yl)morpholine as a light yellow oil (13.0 g, 98.7% yield) which was used directly in next step without further isolation and characterization.

### Step 2: Synthesis of 1,4-dichloro-5,7-dihydrofuro[3,4-d]pyridazine

To a 200 mL sealed tube equipped with a stirring bar were added dichloro-1,2,4,5-tetrazine (6.1 g, 40.4 mmol, 1.0 eq.) in DCM (100 mL). The resulting solution was stirred at 0° C. for 0.5 h. Then 4-(2,5-dihydrofuran-3-yl)morpholine (12.3 g, 79.3 mmol, ~2 eq.) was slowly added into the mixture. The resulting solution was stirred at 0° C. with warming to ~25° C. for 2 h. The reaction mixture was concentrated directly under reduced pressure using a rotary evaporator to obtain a brown oil. The oil was purified by reverse phase column with the following conditions: Column: XSelect CSH Prep C18 OBD Column, 19*150 mm, 5 μm; Mobile Phase A: Water (0.1 vol-% TFA), Mobile Phase B: ACN; Flow rate: 60 mL/min; Gradient: 0% B to 30% B in 30 min, 40% B; Wavelength: 220 nm; RT1: 23.1 min. After concentration of the combined product containing fractions under reduced pressure using a rotary evaporator, 1,4-dichloro-5H,7H-furo[3,4-d]pyridazine (3.0 g, 19.8%) were obtained as a yellow solid. LC/MS: MS (ESI) calcd for C₆H₄Cl₂N₂O: 191.01. Found: m/z=192.10 [M+H]⁺. ¹H NMR (300 MHz, DMSO-d₆): δ 5.21 (s, 4H) ppm.

### Step 3: Synthesis of tert-butyl (3R)-3-({4-chloro-5H,7H-furo[3,4-d]pyridazin-1-yl}amino)piperidine-1-carboxylate

To a solution of 1,4-dichloro-5H,7H-furo[3,4-d]pyridazine (550 mg, 2.88 mmol, 1.0 eq.) in NMP (2 mL)) were added tert-butyl (3R)-3-aminopiperidine-1-carboxylate (577 mg, 2.88 mmol, 1.0 eq.), and DIEA (1.00 mL, 744.32 mg, 5.76 mmol, 2.0 eq.). The reaction mixture was stirred for 2 h at 150° C. in a sealed tube and an atmosphere of N₂. After completion of reaction, the reaction mixture was quenched by addition of water (10 mL). The aqueous layer was extracted with EtOAc (3×50 mL). The combined organic phases were washed with brine (3×50 mL), dried over anhydrous Na₂SO₄, filtered, and the filtrate was concentrated under reduced pressure using a rotary evaporator to yield the crude reaction product which was further purified by silica gel column chromatography using the following conditions Column: silica gel Column; Mobile Phase A: PE, Mobile Phase B: EA; Flow rate: 40 mL/min; Gradient: 0% B to 50% B in 30 min; Wavelength: 254 nm. After concentration of the product containing fractions under reduced pressure using a rotary evaporator, tert-butyl (3R)-3-({4-chloro-5H,7H-furo[3,4-d]pyridazin-1-yl}amino)piperidine-1-carboxylate (290 mg, 28.4%) was obtained as a solid. LC/MS: MS (ESI) calcd For C₁₆H₂₃ClN₄O₃: 354.84. Found: m/z=355.15 [M+H]⁺.

### Step 4: Synthesis of tert-butyl (3R)-3-({4-[2-hydroxy-4-(trifluoromethyl)phenyl]-5H,7H-furo[3,4-d]pyridazin-1-yl}amino)piperidine-1-carboxylate

To a solution of tert-butyl (3R)-3-({4-chloro-5H,7H-furo[3,4-d]pyridazin-1-yl}amino)piperidine-1-carboxylate (290 mg, 0.82 mmol, 1.0 eq.) and 2-hydroxy-4-(trifluoromethyl)phenylboronic acid (253 mg, 1.23 mmol, 1.5 eq.) in 3.75 mL of a mixture of 1,4-dioxane and water (4:1, v/v) were added NaHCO₃ (206 mg, 2.45 mmol, 3 eq.) and Pd(PPh₃)₄ (47.2 mg, 0.041 mmol, 0.05 eq.). After stirring for 4 h at 100° C. in a sealed tube under an atmosphere of N₂, the reaction was quenched with a saturated aqueous NH₄Cl solution (5 mL). The resulting mixture was extracted with EtOAc (3×10 mL). The combined organic layers were washed with H₂O (2×5 mL), and dried over anhydrous Na₂SO₄. After filtration, the filtrate was concentrated under reduced pressure using a rotary evaporator. The residue was purified by silica gel column chromatography using the following conditions: Column: silica gel Column; Mobile Phase A: PE, Mobile Phase B: EA; Flow rate: 60 mL/min; Gradient: 0% B to 50% B in 30 min; Wavelength: 254 nm. After concentration of the combined product containing fractions, tert-butyl (3R)-3-({4-[2-hydroxy-4-(trifluoromethyl)phenyl]-5H,7H-furo[3,4-d]pyridazin-1-yl}amino)piperidine-1-carboxylate (180 mg, 45.8%) was obtained as a solid. LC/MS: MS (ESI) calcd For C₂₃H₂₇F₃N₄O₄: 480.40. Found: m/z=481.10 [M+H]⁺.

### Step 5: Synthesis of 2-{4-[(3R)-piperidin-3-ylamino]-5H, 7H-furo[3,4-d]183yridazine-1-yl}-5-(trifluoromethyl)phenol

To a solution of tert-butyl (3R)-3-({4-[2-hydroxy-4-(trifluoromethyl)phenyl]-5H,7H-furo[3,4-d]pyridazine-1-yl}amino)piperidine-1-carboxylate (180 mg, 0.38 mmol, 1 eq.) in 2.0 mL of 1,4-dioxane, a 4 M solution of HCl in 1,4-dioxane (2 mL, 8 mmol) was added. The reaction mixture was stirred at 30 min at rt. the resulting mixture was concentrated under reduced pressure using a rotary evaporator to afford 2-{4-[(3R)-piperidin-3-ylamino]-5H,7H-furo[3,4-d]pyridazinezin-1-yl}-5-(trifluoromethyl)phenol (130 mg, 91.2%) as a solid. LC/MS: MS (ESI) calcd for C₁₈H₁₉F₃N₄O₂: 380.37. Found: m/z=381.20 [M+H]⁺.

### Step 6: Synthesis of [(3R)-3-({4-[2-hydroxy-4-(trifluoromethyl)phenyl]-5H, 7H-furo[3,4-d]pyridazin-1-yl}amino)piperidin-1-yl]acetic Acid

To a stirred solution of glyoxylate (19.0 mg, 0.26 mmol, 1.5 eq.) in MeOH (2 mL,) were added 2-{4-[(3R)-piperidin-3-ylamino]-5H,7H-furo[3,4-d]pyridazin-1-yl}-5-(trifluoromethyl)phenol (65 mg, 0.17 mmol, 1.0 eq.) and NaBH₃CN (32.2 mg, 0.51 mmol, 3.0 eq.). The reaction mixture was stirred at rt for 24 h. After completion of reaction, the resulting mixture was concentrated under reduced pressure using a rotary evaporator to afford [(3R)-3-({4-[2-hydroxy-4-(trifluoromethyl)phenyl]-5H,7H-furo[3,4-d]pyridazin-1-yl}amino)piperidin-1-yl]acetic acid (50 mg, 66.74%). LC/MS: MS (ESI) calcd for C₂₀H₂₁F₃N₄O₄: 438.40. Found: m/z=439.20 [M+H]⁺.

### Step 7: Synthesis of 2-[(3R)-3-({4-[2-hydroxy-4-(trifluoromethyl)phenyl]-5H, 7H-furo[3,4-d]pyridazin-1-yl}amino)piperidin-1-yl]-1-(4-hydroxypiperidin-1-yl)ethanone (23)

To a solution of [(3R)-3-({4-[2-hydroxy-4-(trifluoromethyl)phenyl]-5H,7H-furo[3,4-d]pyridazin-1-yl}amino)piperidin-1-yl]acetic acid (50 mg, 0.11 mmol, 1.0 eq.) and piperidin-4-ol (20.8 mg, 0.21 mmol, 1.8 eq.) in DMF (1 mL) was treated with HOBT (30.8 mg, 0.23 mmol, 2.0 eq.) and DIEA (59.6 μL, 44.2 mg, 0.34 mmol, 3.0 eq.). The reaction mixture was stirred for 24 h at rt. The reaction progress was monitored by TLC (EA, Rf=0.3). After completion of reaction, the resulting mixture was concentrated under reduced pressure using a rotary evaporator. The crude reaction mixture was purified by preparative RP-HPLC using the following conditions Column: XBridge Prep OBD C18 Column, 30*150 mm, 5 μm; Mobile Phase A: 10 mmol NH₄HCO₃), Mobile Phase B: ACN; Flow rate: 60 mL/min; Gradient: 30% B to 58% B in 60 min, 58% B; Wavelength: 254 nm; RT1: 7 min. After lyophilization of the combined product containing fractions, the target compound 2-[(3R)-3-({4-[2-hydroxy-4-(trifluoromethyl)phenyl]-5H,7H-furo[3,4-d]pyridazin-1-yl}amino)piperidin-1-yl]-1-(4-hydroxypiperidin-1-yl) ethenone (23) (20.4 mg, 34.0% yield) was obtained as a light yellow solid. LC/MS: mass calcd for C₂₅H₃₀F₃N₅O₄: 521.22, found: m/z=522.25 [M+H]⁺. ¹H NMR (400 MHz, Methanol-d₄): δ 7.42-7.40 (m, 1H), 7.16-7.10 (m, 2H), 5.21-5.13 (m, 2H), 4.93 (s, 1H), 4.20 (s, 1H), 3.99-3.90 (m, 1H), 3.75-3.70 (m, 1H), 3.51-2.97 (m, 5H), 2.71-2.69 (m, 1H), 2.31-1.90 (m, 4H), 1.75-1.56 (m, 4H), 1.40-1.13 (m, 3H) ppm. ¹⁹F NMR (376 MHz, Methanol-d₄): δ −64.39 ppm.

### Example 24: (R)-2-(3-((4-(2-Hydroxy-4-(trifluoromethyl)phenyl)-5,7-dihydrofuro[3,4-d]pyridazin-1-yl)amino)piperidin-1-yl)-1-(2-hydroxy-7-azaspiro[3.5]nonan-7-yl)ethan-1-one (24)

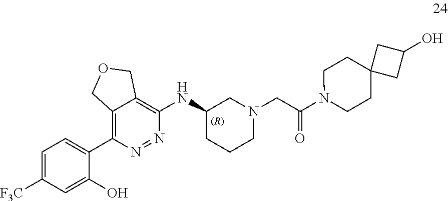

### Synthetic Scheme:

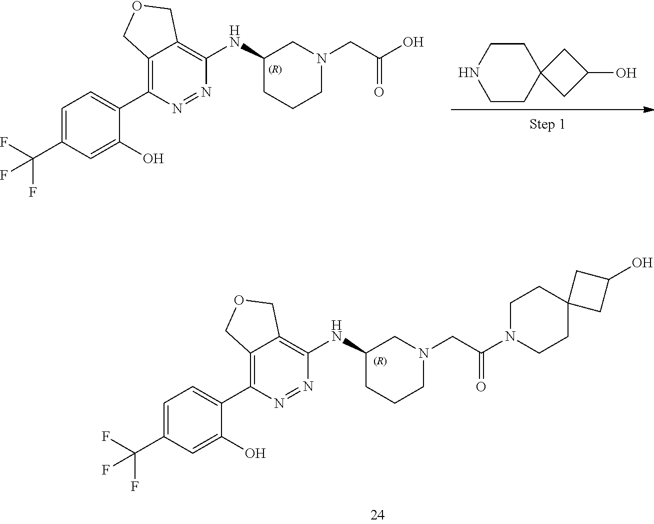

### Step 1: Synthesis of (R)-2-(3-((4-(2-hydroxy-4-(trifluoromethyl)phenyl)-5,7-dihydrofuro[3,4-d]pyridazin-1-yl)amino)piperidin-1-yl)-1-(2-hydroxy-7-azaspiro[3.5]nonan-7-yl)ethan-1-one (24)

To a light yellow solution [(3R)-3-({4-[2-hydroxy-4-(trifluoromethyl)phenyl]-5H,7H-furo[3,4-d]pyridazin-1-yl}amino)piperidin-1-yl]acetic acid (prepared as disclosed in Example 23) (60 mg, 0.14 mmol, 1.0 eq.) in DMF (2 mL), 7-azaspiro[3.5]nonan-2-ol (25.1 mg, 0.18 mmol, 1.3 eq.), EDCI (52.5 mg, 0.274 mmol, 2.0 eq.), HOBT (37.0 mg, 0.274 mmol, 2.0 eq.), and DIEA (71.5 μL, 53.1 mg, 0.41 mmol, 3 eq.) were added under stirring at rt. The suspension was stirred for 4 h at rt resulting in a brown suspension. The reaction was quenched at 0° C. through the addition of a saturated aqueous NH₄Cl solution (8 mL). The resulting mixture was extracted with EtOAc (3×20 mL). The combined organic layers were washed with water (3×20 mL), and dried over anhydrous Na₂SO₄. After filtration, the filtrate was concentrated under reduced pressure using a rotary evaporator. The crude reaction product was purified by preparative RP-HPLC using the following conditions Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 37% B to 64% B in 10 min, 64% B; Wavelength: 254 nm; RT1: 7.18 min; Injection Volume: 1.6 mL; Number of runs: 1 to afford the target compound (R)-2-(3-((4-(2-hydroxy-4-(trifluoromethyl)phenyl)-5,7-dihydrofuro[3,4-d]pyridazin-1-yl)amino)piperidin-1-yl)-1-(2-hydroxy-7-azaspiro[3.5]nonan-7-yl)ethan-1-one (24) (11.8 mg, 14.8% yield) as an off-white solid after lyophilization of the combined product containing fractions. LC/MS: MS (ESI) calcd for C₂₈H₃₄F₃N₅O₄: 561.26, found: m/z=562.25 [M+H]⁺. ¹H NMR (300 MHz, Methanol-d₄): δ 7.41-7.33 (m, 1H), 7.22-7.18 (m, 2H), 5.39-5.37 (s, 2H), 5.04 (s, 2H), 4.45-4.13 (m, 2H), 3.73-3.38 (m, 4H), 3.26-3.24 (m, 1H), 3.19-3.15 (m, 1H), 3.10-2.92 (m, 1H), 2.69-2.57 (m, 1H), 2.42-2.19 (m, 4H), 2.03-1.44 (m, 10H) ppm. ¹⁹F NMR (282 MHz, Methanol-d₄): δ −64.55 ppm.

### Example 25: (R)-2-(3-((4-(2-Hydroxy-4-(trifluoromethyl)phenyl)-5,7-dihydrofuro[3,4-d]pyridazin-1-yl)amino)piperidin-1-yl)-1-(7-hydroxy-2-azaspiro[3.5]nonan-2-yl)ethan-1-one (25)

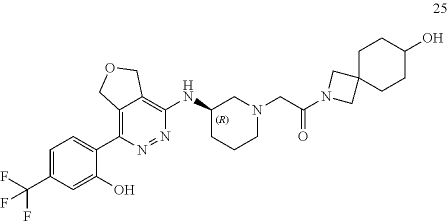

### Synthetic Scheme

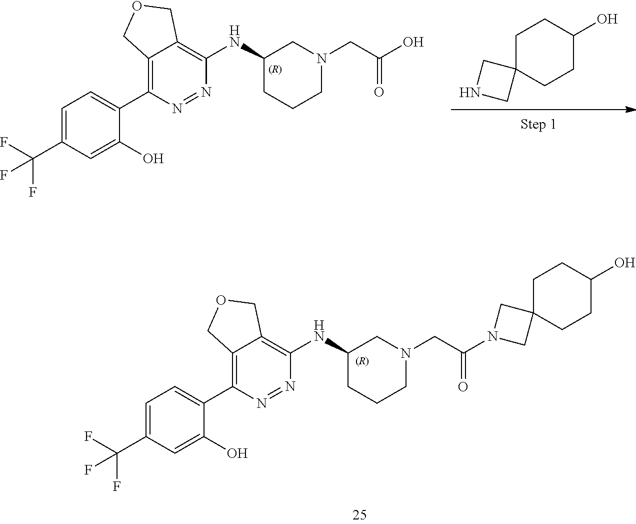

### Step 1: Synthesis of (R)-2-(3-((4-(2-hydroxy-4-(trifluoromethyl)phenyl)-5,7-dihydrofuro[3,4-d]pyridazin-1-yl)amino)piperidin-1-yl)-1-(7-hydroxy-2-azaspiro[3.5]nonan-2-yl)ethan-1-one (25)

To a light yellow solution of [(3R)-3-({4-[2-hydroxy-4-(trifluoromethyl)phenyl]-5H,7H-furo[3,4-d]pyridazin-1-yl}amino)piperidin-1-yl]acetic acid (prepared as disclosed in Example 23) (50 mg, 0.114 mmol, 1.0 eq.) in DMF (2 mL), 2-azaspiro[3.5]nonan-7-ol (19.3 mg, 0.137 mmol, 1.2 eq.), EDCI (44 mg, 0.23 mmol, 2 eq.), HOBt (30.8 mg, 0.23 mmol, 2.0), and DIEA (60 μL, 44.2 mg, 0.342 mmol, 3 eq.) were added under stirring at rt. The suspension was stirred for 4 h at rt resulting in a brown suspension. The reaction was quenched at 0° C. (ice bath) through the addition of a saturated aqueous NH₄Cl solution (8 mL). The resulting mixture was extracted with EtOAc (3×20 mL). The combined organic layers were washed with water (3×20 mL) and dried over anhydrous Na₂SO₄. After filtration, the filtrate was concentrated under reduced pressure using a rotary evaporator. The residue was purified by preparative RP-HPLC using the following conditions Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 31% B to 61% B in 7 min, 61% B; Wavelength: 254 nm; RT1: 6.15 min; Injection Volume: 1 mL; Number of runs: 2 to yield the target compound (R)-2-(3-((4-(2-hydroxy-4-(trifluoromethyl)phenyl)-5,7-dihydrofuro[3,4-d]pyridazin-1-yl)amino)piperidin-1-yl)-1-(7-hydroxy-2-azaspiro[3.5]nonan-2-yl)ethan-1-one (25) (13.0 mg, 20.3% yield) as an off-white solid after lyophilization of the combined product containing fractions. LC/MS: MS (ESI) calcd for C₂₈H₃₄F₃N₅O₄: 561.26, found: m/z=562.30 [M+H]⁺. ¹H NMR (300 MHz, Methanol-d₄): δ 7.40-7.32 (m, 1H), 7.20-7.16 (m, 2H), 5.37 (s, 2H), 5.05 (s, 2H), 4.63-4.59 (m, 1H), 4.41-4.33 (m, 1H), 4.13-3.90 (m, 2H), 3.71-3.48 (m, 3H), 3.18-3.08 (m, 2H), 2.99-2.87 (m, 1H), 2.62-2.52 (m, 1H), 2.48-2.26 (m, 2H), 2.08-1.49 (m, 10H), 1.41-1.27 (m, 2H) ppm. ¹⁹F NMR (282 MHz, Methanol-d₄): δ −64.52 ppm.

### Example 26: 2-((R)-3-((4-(2-Hydroxy-4-(trifluoromethyl)phenyl)-5,7-dihydrofuro[3,4-d]pyridazin-1-yl)amino)piperidin-1-yl)-1-((S)-3-hydroxypyrrolidin-1-yl)ethan-1-one (26)

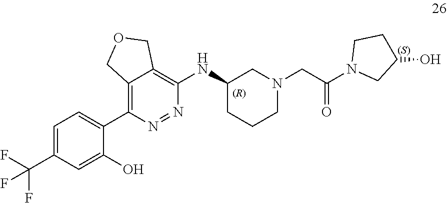

### Synthetic Scheme:

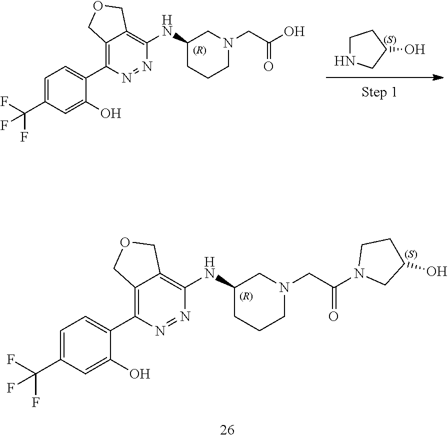

### Step 1: Synthesis of 2-((R)-3-((4-(2-hydroxy-4-(trifluoromethyl)phenyl)-5,7-dihydrofuro[3,4-d]pyridazin-1-yl)amino)piperidin-1-yl)-1-((S)-3-hydroxypyrrolidin-1-yl)ethan-1-one (26)

To a light yellow solution of [(3R)-3-({4-[2-hydroxy-4-(trifluoromethyl)phenyl]-5H,7H-furo[3,4-d]pyridazine-1-yl}amino)piperidin-1-yl]acetic acid (prepared as disclosed in Example 23) (100 mg, 0.228 mmol, 1 eq.) in DMF (2 mL), (3S)-pyrrolidin-3-ol (23.9 mg, 0.274 mmol, 1.2 eq.), EDCI (88 mg, 0.46 mmol, 2 eq.), HOBt (61.6 mg, 0.456 mmol, 2 eq.), and DIEA (119.2 μL, 88.44 mg, 0.684 mmol, 3.0 eq) were added under stirring at rt. The suspension was stirred for 4 h at rt resulting in a brown suspension. The reaction was quenched at 0° C. through addition of a saturated aqueous NH₄Cl solution (8 mL). The resulting mixture was extracted with EtOAc (3×20 mL). The combined organic layers were washed with water (3×20 mL), dried over anhydrous Na₂SO₄. After filtration, the filtrate was concentrated under reduced pressure using a rotary evaporator. The residue was purified by preparative RP-HPLC using the following conditions Column: Xselect CSH C18 OBD Column 30*150 mm 5 μm; Mobile Phase A: Water (0.1 vol-% FA), Mobile Phase B: can; Flow rate: 60 mL/min; Gradient: 10% B to 40% B in 7 min, 40% B; Wavelength: 254 nm; RT1: 6.12 min; Injection Volume: 0.67 mL; Number of runs: 3 to afford the target compound 2-((R)-3-((4-(2-hydroxy-4-(trifluoromethyl)phenyl)-5,7-dihydrofuro[3,4-d]pyridazin-1-yl)amino)piperidin-1-yl)-1-((S)-3-hydroxypyrrolidin-1-yl)ethan-1-one (26) (9.3 mg, 8.0% yield) as an off-white solid after lyophilization of the combined product containing fractions. LC/MS: MS (ESI) calcd for C₂₄H₂₈F₃N₅O₄: 507.20, found: m/z=508.25 [M+H]⁺. ¹H NMR (300 MHz, Methanol-d₄): δ 7.40-7.31 (m, 1H), 7.21-7.16 (m, 2H), 5.37 (s, 2H), 5.07 (s, 2H), 4.47-4.41 (m, 2H), 4.33-4.17 (m, 1H), 3.72-3.52 (m, 3H), 3.50-3.47 (m, 1H), 3.28-3.13 (m, 2H), 2.94-2.89 (m, 1H), 2.66-2.45 (m, 3H), 2.10-1.83 (m, 4H), 1.72-1.58 (m, 2H) ppm. ¹⁹F NMR (282 MHz, Methanol-d₄): δ −64.55 ppm.

### Example 27: 2-((R)-3-((4-(2-Hydroxy-4-(trifluoromethyl)phenyl)-5,7-dihydrofuro[3,4-d]pyridazin-1-yl)amino)piperidin-1-yl)-1-((R)-3-hydroxypyrrolidin-1-yl)ethan-1-one (27)

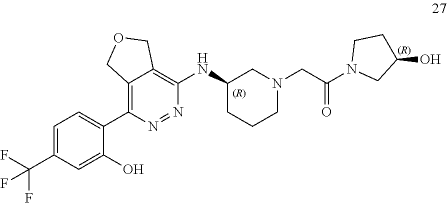

### Synthetic Scheme:

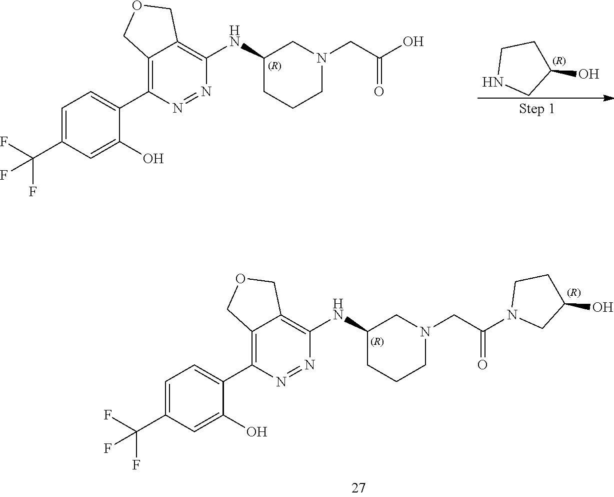

### Step 1: Synthesis of 2-((R)-3-((4-(2-hydroxy-4-(trifluoromethyl)phenyl)-5,7-dihydrofuro[3,4-d]pyridazin-1-yl)amino)piperidin-1-yl)-1-((R)-3-hydroxypyrrolidin-1-yl)ethan-1-one (27)

To a light yellow solution of [(3R)-3-({4-[2-hydroxy-4-(trifluoromethyl)phenyl]-5H,7H-furo[3,4-d]pyridazin-1-yl}amino)piperidin-1-yl]acetic acid (prepared as disclosed in Example 23) (100 mg, 0.23 mmol, 1 eq.) in DMF (2 mL), (3R)-pyrrolidin-3-ol (23.9 mg, 0.27 mmol, 1.2 eq.), EDCI (87.5 mg, 0.46 mmol, 2.0 eq.), HOBT (61.6 mg, 0.46 mmol, 2.0 eq.), and DIEA (119.2 μL, 88.44 mg, 0.68 mmol, 3.0 eq.) were added under stirring at rt. The suspension was stirred for 4 h at rt resulting in a brown suspension. The reaction was quenched at 0° C. through the addition of a saturated aqueous NH₄Cl solution (8 mL). The resulting mixture was extracted with EtOAc (3×20 mL). The combined organic layers were washed with water (3×20 mL), and dried over anhydrous Na₂SO₄. After filtration, the filtrate was concentrated under reduced pressure using a rotary evaporator. The residue was purified by preparative RP-HPLC using the following conditions Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 33% B to 63% B in 7 min, 63% B; Wavelength: 254 nm; RT1: 6.63 min; Injection Volume: 0.9 mL; Number of runs: 2 to afford the target compound 2-((R)-3-((4-(2-hydroxy-4-(trifluoromethyl)phenyl)-5,7-dihydrofuro[3,4-d]pyridazin-1-yl)amino)piperidin-1-yl)-1-((R)-3-hydroxypyrrolidin-1-yl) ethan-1-one (27) (12.0 mg, 10.4% yield) as a colorless solid after lyophilization of the combined product containing fractions. LC/MS: MS (ESI) calcd for C₂₄H₂₈F₃N₅O₄: 507.21, found: m/z=508.25 [M+H]⁺. ¹H NMR (300 MHz, Methanol-d₄): δ 7.42-7.39 (m, 1H), 7.20-7.18 (m, 2H), 5.39-5.37 (m, 2H), 5.07 (s, 2H), 4.46-4.37 (m, 2H), 3.70-3.67 (m, 3H), 3.57-3.48 (m, 1H), 3.25-3.21 (m, 2H), 2.99-2.83 (m, 1H), 2.63-2.93 (m, 3H), 2.20-1.79 (m, 4H), 1.77-1.56 (m, 2H) ppm. ¹⁹F NMR (282 MHz, Methanol-d₄): δ −64.55 ppm.

### Example 28: 1-((3S,4S)-3-Fluoro-4-hydroxypyrrolidin-1-yl)-2-((R)-3-((4-(2-hydroxy-4-(trifluoromethyl)phenyl)-5,7-dihydrofuro[3,4-d]pyridazin-1-yl)amino)piperidin-1-yl)ethan-1-one (28)

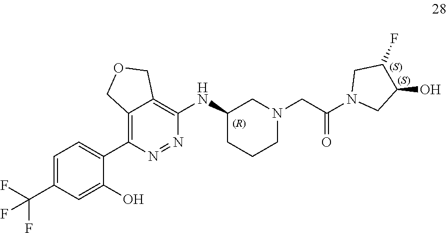

### Synthetic Scheme:

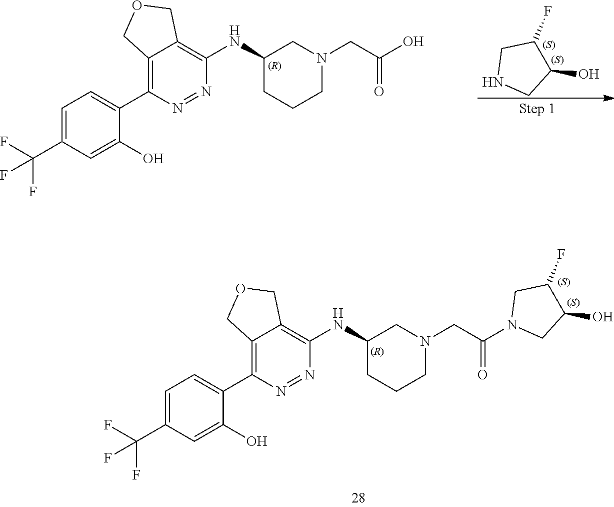

### Step 1: Synthesis of 1-[(3S,4S)-3-fluoro-4-hydroxypyrrolidin-1-yl]-2-[(3R)-3-({4-[2-hydroxy-4-(trifluoromethyl)phenyl]-5H, 7H-furo[3,4-d]pyridazin-1-yl}amino)piperidin-1-yl]ethenone (28)

To a light yellow solution of [(3R)-3-({4-[2-hydroxy-4-(trifluoromethyl)phenyl]-5H,7H-furo[3,4-d]pyridazin-1-yl}amino)piperidin-1-yl]acetic acid (prepared as disclosed in Example 23) (100 mg, 0.228 mmol, 1.0 eq.) in DMF (2 mL), (3S,4S)-4-fluoropyrrolidin-3-ol (29 mg, 0.27 mmol, 1.2 eq.), EDCI (88 mg, 0.46 mmol, 2.0 eq.), HOBt (62 mg, 0.46 mmol, 2.0 eq.), and DIEA (120 μL, 88.4 mg, 0.68 mmol, 3.0 eq.) were added under stirring at rt. The suspension was stirred for 4 h at rt resulting in a brown suspension. The reaction was quenched at 0° C. through the addition of a saturated aqueous NH₄Cl solution (8 mL). The resulting mixture was extracted with EtOAc (3×20 mL). The combined organic layers were washed with water (3×20 mL), and dried over anhydrous Na₂SO₄. After filtration, the filtrate was concentrated under reduced pressure using rotary evaporator. The residue was purified by preparative RP-HPLC using the following conditions: Column: XBridge Prep Phenyl OBD Column, 19*250 mm, 5 μm; Mobile Phase A: Water (10 mmol/L NH₄HCO₃), Mobile Phase B: ACN; Flow rate: 25 mL/min; Gradient: 33% B to 63% B in 7 min, 63% B; Wavelength: 254 nm; RT1: 6.63 min; Injection Volume: 0.9 mL; Number of runs: 2 to afford the target compound 1-[(3S,4S)-3-fluoro-4-hydroxypyrrolidin-1-yl]-2-[(3R)-3-({4-[2-hydroxy-4-(trifluoromethyl)phenyl]-5H,7H-furo[3,4-d]pyridazin-1-yl}amino)piperidin-1-yl]ethanone (28) (15.7 mg, 12.6% yield) as a light yellow solid after lyophilization of the combined product containing fractions. LC/MS: MS (ESI) calcd for C₂₄H₂₇F₄N₅O₄: 525.20, found: m/z=526.20 [M+H]⁺. ¹H NMR (300 MHz, Methanol-d₄): 7.42-7.39 (m, 1H), 7.20-7.18 (m, 2H), 5.37 (s, 2H), 5.08-5.06 (m, 2H), 5.01-4.89 (m, 1H), 4.52-4.27 (m, 2H), 4.05-3.83 (m, 1H), 3.79-3.51 (m, 3H), 3.23-3.20 (m, 2H), 3.17-2.88 (m, 1H), 2.82-2.79 (m, 3H), 1.85-1.90 (m, 2H), 1.76-1.54 (m, 2H) ppm. ¹⁹F NMR (282 MHz, Methanol-d₄): δ −64.54, −185.54 ppm.

### Example 29: 1-((3R,4R)-3-Fluoro-4-hydroxypyrrolidin-1-yl)-2-((R)-3-((4-(2-hydroxy-4-(trifluoromethyl)phenyl)-5,7-dihydrofuro[3,4-d]pyridazin-1-yl)amino)piperidin-1-yl)ethan-1-one (29)

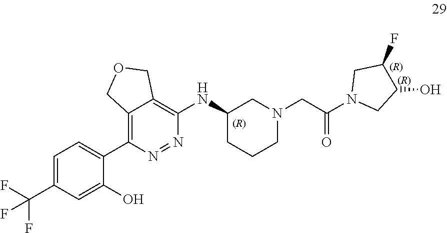

### Synthetic Scheme:

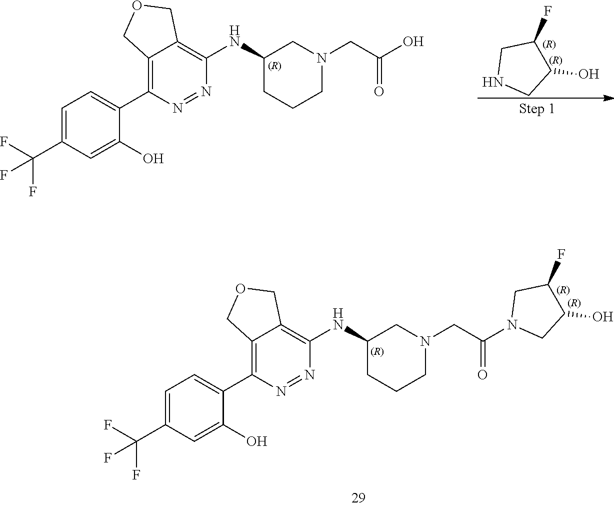

Step 1: Synthesis of 1-((3R,4R)-3-fluoro-4-hydroxypyrrolidin-1-yl)-2-((R)-3-((4-(2-hydroxy-4-(trifluoromethyl)phenyl)-5,7-dihydrofuro[3,4-d]pyridazin-1-yl)amino)piperidin-1-yl)ethan-1-one (29)

To a light yellow solution of [(3R)-3-({4-[2-hydroxy-4-(trifluoromethyl)phenyl]-5H,7H-furo[3,4-d]pyridazin-1-yl}amino)piperidin-1-yl]acetic acid (prepared as disclosed in Example 23) (150 mg, 0.34 mmol, 1.0 eq.) in DMF (2 mL), (3R,4R)-4-fluoropyrrolidin-3-ol (50 mg, 0.514 mmol, 1.5 eq.), HATU (156 mg, 0.41 mmol, 1.2 eq.), and DIEA (179.2 μL, 133 mg, 1.03 mmol, 3.0 eq.) were added under stirring at rt. The brown suspension was stirred for 4 h at room temperature. The reaction was quenched at 0° C. through the addition of a saturated aqueous NH₄Cl solution (8 mL). The resulting mixture was extracted with EtOAc (3×20 mL). The combined organic layers were washed with water (3×20 mL), and dried over anhydrous Na₂SO₄. After filtration, the filtrate was concentrated under reduced pressure using a rotary evaporator. The residue was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 36% B to 51% B in 10 min, 51% B; Wavelengths: 254/220 nm; RT1: 7.78 min. After lyophilization of the combined product containing fractions, the target compound 1-((3R,4R)-3-fluoro-4-hydroxypyrrolidin-1-yl)-2-((R)-3-((4-(2-hydroxy-4-(trifluoromethyl)phenyl)-5,7-dihydrofuro[3,4-d]pyridazin-1-yl)amino)piperidin-1-yl)ethan-1-one (29) was obtained (14.3 mg, 5.3% yield) as a light yellow solid. LC/MS: MS (ESI) calcd for C₂₄H₂₇F₄N₅O₄: 525.20, found: m/z=526.25 [M+H]⁺. ¹H-NMR (400 MHz, Methanol-d₄): δ 7.42-7.32 (m, 1H), 7.22-7.10 (m, 2H), 5.35 (s, 2H), 5.10-4.90 (m, 3H), 4.50-4.25 (m, 2H), 4.00-3.51 (m, 4H), 3.27 (s, 2H), 3.01-2.85 (m, 1H), 2.65-2.40 (m, 3H), 2.00-1.80 (m, 2H), 1.80-1.50 (m, 2H) ppm. ¹⁹F NMR (376 MHz, Methanol-d₄): δ −64.50, −184.62 ppm.

### Example 30: (R)-2-(3-((6-(2-Hydroxy-4-(trifluoromethyl)phenyl)-4,5-dimethylpyridazin-3-yl)amino)piperidin-1-yl)-1-(4-hydroxypiperidin-1-yl)ethan-1-one (30)

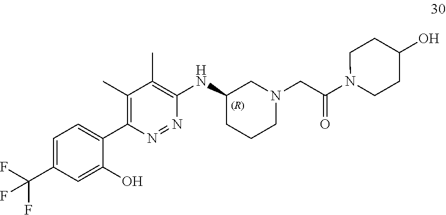

### Synthetic Scheme

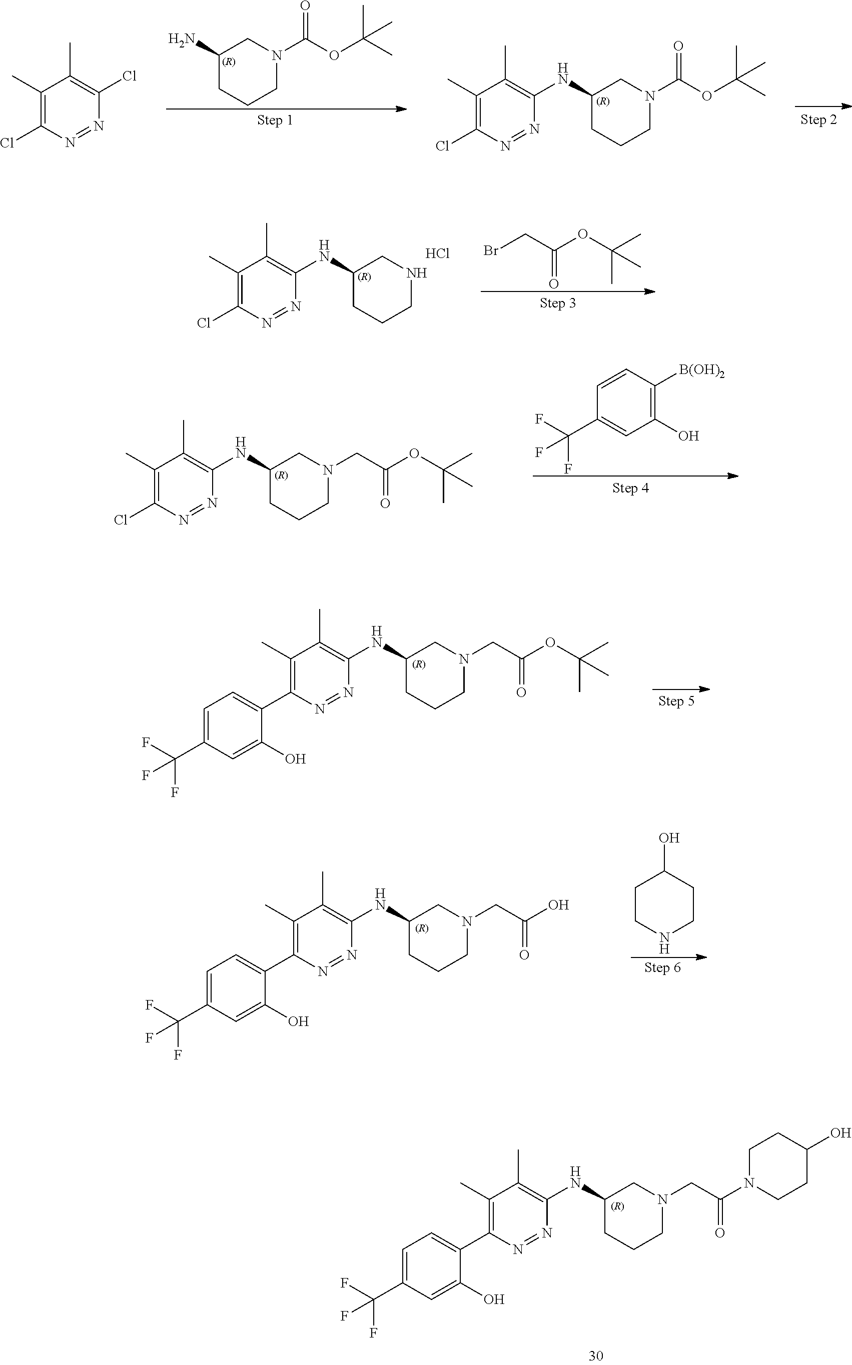

### Step 1: Synthesis of tert-butyl (R)-3-((6-chloro-4,5-dimethylpyridazin-3-yl)amino)piperidine-1-carboxylate

In a sealed tube with 3,6-dichloro-4,5-dimethylpyridazine (5.0 g, 28.2 mmol, 1.0 eq.), tert-butyl (R)-3-aminopiperidine-1-carboxylate (6.2 g, 28.2 mmol, 1.0 eq) and DIEA (14.7 mL, 10.9 g, 84.6 mmol, 3.0 eq), anhydrous NMP (50.0 mL) was added. The tube was sealed and stirred at 150° C. for overnight. After cooling down to rt, the crude reaction mixture was poured into water (300 mL), and the solution was extracted with EtOAc (3×100 mL), washed with a 5 wt-% aqueous LiCl solution (3×50 mL). All organic layers were combined, dried with anhydrous Na₂SO₄, filtered, and the filtrate was concentrated under reduced pressure using a rotary evaporator to afford the crude reaction product. The crude reaction product was purified using a Biotage Isolera equipped with a Biotage SFAR (40 g, HC, Duo, 20 μM) with 0-5% MeOH/DCM gradients. The combined product containing fractions were concentrated under reduced pressure using a rotary evaporator to yield tert-butyl (R)-3-((6-chloro-4,5-dimethylpyridazin-3-yl)amino)piperidine-1-carboxylate (1.0 g, 10.4% yield), as a pale-yellow solid. LC/MS: m/z=341.15 [M+H]⁺. ¹H NMR (400 MHz, Methanol-d₄): δ 4.32-4.29 (m, 1H), 3.63-3.59 (m, 1H), 3.45-3.42 (m, 1H), 3.30-3.17 (m, 3H), 3.04-3.00 (m, 1H), 2.48 (s, 3H), 2.39 (d, J=0.9 Hz, 3H), 2.23-2.05 (m, 2H), 2.00-1.81 (m, 2H), 1.44 (s, 9H) ppm.

### Step 2: Synthesis of (R)-6-chloro-4,5-dimethyl-N-(piperidin-3-yl)pyridazin-3-amine

In a flask containing tert-butyl (R)-3-((6-chloro-4,5-dimethylpyridazin-3-yl)amino)piperidine-1-carboxylate (1.0 g, 2.9 mmol, 1.0 eq), the chemical was dissolved in anhydrous dioxane (5.0 mL). To the solution was added a solution of HCl (5.0 mL, 20 mmol, 4 M in 1,4-dioxane) and the reaction mixture was stirred at rt for 1 h. The reaction mixture was concentrated under reduced pressure using a rotary evaporator to yield the crude reaction product. The crude reaction product was purified using a Biotage Isolera equipped with a Biotage SFAR (4 g, HC, Duo, 20 μM) and 0-10% MeOH/DCM gradients. The combined product containing fractions were concentrated under reduced pressure using a rotary evaporator to yield (R)-6-chloro-4,5-dimethyl-N-(piperidin-3-yl)pyridazin-3-amine (580 mg, 82.0% yield), as a colorless solid. LC/MS: m/z=241.15 [M+H]⁺. ¹H NMR (400 MHz, Methanol-d₄): δ 4.27 (td, J=10.1, 4.9 Hz, 1H), 3.59 (dd, J=12.5, 4.1 Hz, 1H), 3.38 (dt, J=12.8, 3.9 Hz, 1H), 3.30-3.17 (m, 3H), 3.04 (td, J=12.1, 3.5 Hz, 1H), 2.48 (s, 3H), 2.39 (d, J=0.9 Hz, 3H), 2.23-2.05 (m, 2H), 2.00-1.81 (m, 2H) ppm.

### Step 3: Synthesis of tert-butyl (R)-2-(3-((6-chloro-4,5-dimethylpyridazin-3-yl)amino)piperidin-1-yl)acetate

In a flask containing (R)-6-chloro-4,5-dimethyl-N-(piperidin-3-yl)pyridazin-3-amine (570 mg, 2.4 mmol, 1.0 eq.), t-butyl bromoacetate (462 mg, 2.4 mmol, 1.0 eq.), and K₂CO₃ (655 mg, 4.8 mmol, 2.0 eq.), anhydrous ACN (5 mL) was added. The reaction mixture was stirred in a sealed tube at 80° C. for overnight. After cooling down to rt, the crude reaction mixture was poured into water (100 mL), and the solution was extracted with EtOAc (3×20 mL). All organic layers were combined, dried with anhydrous Na₂SO₄, filtered, and the filtrate was then concentrated under reduced pressure using a rotary evaporator to yield the crude reaction product. The crude reaction product was purified using a Biotage Isolera equipped with a Biotage SFAR (4 g, HC, Duo, 20 μM) using 0-4% MeOH/DCM gradients. The combined product containing fractions were concentrated under reduced pressure using a rotary evaporator to yield tert-butyl (R)-2-(3-((6-chloro-4,5-dimethylpyridazin-3-yl)amino)piperidin-1-yl)acetate (460 mg, 57.1% yield) as a colorless solid. LC/MS: m/z=355.20 [M+H]⁺. ¹H NMR (400 MHz, Methanol-d₄) δ 4.29 (s, 1H), 3.15 (s, 2H), 2.87 (s, 1H), 2.56 (s, 3H), 2.32 (s, 3H), 2.19 (s, 3H), 1.77 (s, 2H), 1.65 (s, 2H), 1.46 (s, 9H) ppm.

### Step 4: Synthesis of tert-butyl (R)-2-(3-((6-(2-hydroxy-4-(trifluoromethyl)phenyl)-4,5-dimethylpyridazin-3-yl)amino)piperidin-1-yl)acetate

In a sealed tube containing tert-butyl (R)-2-(3-((6-chloro-4,5-dimethylpyridazin-3-yl)amino)piperidin-1-yl)acetate (450 mg, 1.3 mmol, 1.0 eq), (2-hydroxy-4-(trifluoromethyl)phenyl) boronic acid (392 mg, 1.9 mol, 1.5 eq.), Na₂CO₃ (787 mg, 5.7 mmol, 3.0 eq.), and Pd(PPh₃)₄ (231 mg, 0.2 mol, 0.1 eq.), dioxane (4 mL) was added. The reaction mixture was stirred at 110° C. for overnight under an atmosphere of N₂. After cooling down to rt, the crude reaction mixture was poured into water (100 mL), and the solution was extracted with EtOAc (3×20 mL). All organic layers were combined, dried over anhydrous Na₂SO₄, filtered, and the filtrate concentrated under reduced pressure using a rotary evaporator to yield the crude reaction product. The crude product was purified using a Biotage Isolera equipped with a Biotage SFAR (4 g, HC, Duo, 20 μM) using 0-4% MeOH/DCM and then concentrated to give tert-butyl (R)-2-(3-((6-(2-hydroxy-4-(trifluoromethyl)phenyl)-4,5-dimethylpyridazin-3-yl)amino)piperidin-1-yl)acetate (500 mg, 82.1% yield) as a pale-yellow solid. LC/MS: m/z=481.15 [M+H]⁺. ¹H NMR (400 MHz, Methanol-d₄): δ 7.34 (s, 1H), 7.19 (s, 1H), 7.15 (s, 1H), 4.41 (s, 1H), 3.63 (s, 2H), 3.29 (s, 1H), 2.57 (s, 3H), 2.16 (s, 3H), 2.07 (s, 3H), 1.78 (s, 2H), 1.66-1.62 (m, 2H), 1.60-1.55 (m, 4H), 1.45 (s, 9H) ppm.

### Step 5: Synthesis of (R)-2-(3-((6-(2-hydroxy-4-(trifluoromethyl)phenyl)-4,5-dimethylpyridazin-3-yl)amino)piperidin-1-yl)acetic Acid

In a flask containing tert-butyl (R)-2-(3-((6-(2-hydroxy-4-(trifluoromethyl)phenyl)-4,5-dimethylpyridazin-3-yl)amino)piperidin-1-yl)acetate (500 mg, 1.04 mmol, 1.0 eq.), the chemical was dissolved in anhydrous DCM (5.0 mL) and TFA (5.0 mL) was added. The reaction mixture was stirred at rt for 1 h. The mixture was concentrated under reduced pressure using a rotary evaporator to yield the crude reaction product. The crude reaction product was purified using a Biotage Isolera equipped with a Biotage SFAR (4 g, HC, Duo, 20 μM) using 0-40% MeOH/DCM gradients. The combined product containing fractions were concentrated under reduced pressure using a rotary evaporator to yield (R)-2-(3-((6-(2-hydroxy-4-(trifluoromethyl)phenyl)-4,5-dimethylpyridazin-3-yl)amino)piperidin-1-yl)acetic acid (106 mg, 24.0% yield) as a colorless solid. LC/MS: m/z=425.20 [M+H]⁺. ¹H NMR (400 MHz, DMSO-d₆): δ 7.34 (d, J=7.5 Hz, 1H), 7.19 (d, J=8.1 Hz, 2H), 6.08-6.00 (m, 1H), 4.50 (s, 1H), 3.75 (s, 2H), 3.45-3.32 (m, 1H), 3.19 (s, 1H), 2.77-2.62 (m, 2H), 2.47 (s, 4H), 2.06 (s, 3H), 1.95 (s, 3H), 1.82 (s, 3H), 1.63-1.51 (m, 1H) ppm.

### Step 6: Synthesis of (R)-2-(3-((6-(2-hydroxy-4-(trifluoromethyl)phenyl)-4,5-dimethylpyridazin-3-yl)amino)piperidin-1-yl)-1-(4-hydroxypiperidin-1-yl)ethan-1-one (30)

In a flask containing (R)-2-(3-((6-(2-hydroxy-4-(trifluoromethyl)phenyl)-4,5-dimethylpyridazin-3-yl)amino)piperidin-1-yl)acetic acid (106 mg, 0.3 mmol, 1.0 eq.), 4-hydroxypiperidine (28 mg, 0.3 mmol, 1.0 eq.), and EDCI (93 mg, 0.7 mmol, 3.0 eq.), pyridine (2 mL) was added. The reaction mixture was stirred at 50° C. for overnight under an atmosphere of N₂. After cooling down to rt, the crude reaction mixture was concentrated under reduced pressure using a rotary evaporator to yield the crude reaction product. The crude reaction product was purified by preparative RP-HPLC using ACN/water mixtures acidified with 0.3 vol-% FA (5-95% gradients in 30 min) to afford (R)-2-(3-((6-(2-hydroxy-4-(trifluoromethyl)phenyl)-4,5-dimethylpyridazin-3-yl)amino)piperidin-1-yl)-1-(4-hydroxypiperidin-1-yl)ethan-1-one (30) formate salt (37.9 mg, 29.9% recovery yield) after lyophilization of compound containing fractions as a colorless solid. LC/MS: m/z=508.30 [M+H]⁺. ¹H NMR (400 MHz, Methanol-d₄): δ 8.38 (s, 1H), 7.37 (dd, J=7.8, 4.4 Hz, 1H), 7.22 (d, J=7.9 Hz, 1H), 7.16 (s, 1H), 4.47 (s, 1H), 4.10 (s, 2H), 3.85 (d, J=11.0 Hz, 1H), 3.80 (d, J=4.1 Hz, 2H), 3.56 (d, J=14.4 Hz, 1H), 3.37 (s, 1H), 3.17 (s, 2H), 3.05 (d, J=10.4 Hz, 1H), 2.75 (s, 1H), 2.62 (s, 1H), 2.19 (d, J=4.1 Hz, 3H), 2.10 (d, J=2.9 Hz, 3H), 2.02 (s, 2H), 1.92 (d, J=18.7 Hz, 2H), 1.86-1.79 (m, 2H), 1.72 (s, 1H), 1.42 (s, 2H) ppm. ¹⁹F NMR (376 MHz, Methanol-d₄): δ −64.31 (s) ppm.

### Example 31: 2-((R)-3-((6-(2-Hydroxy-4-(trifluoromethyl)phenyl)-4-methylpyridazin-3-yl)amino)piperidin-1-yl)-1-((S)-3-hydroxypyrrolidin-1-yl)ethan-1-one (31)

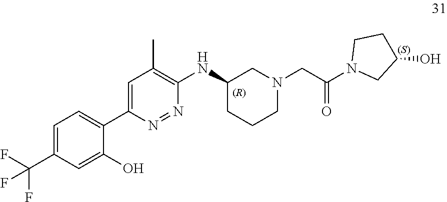

### Synthetic Scheme

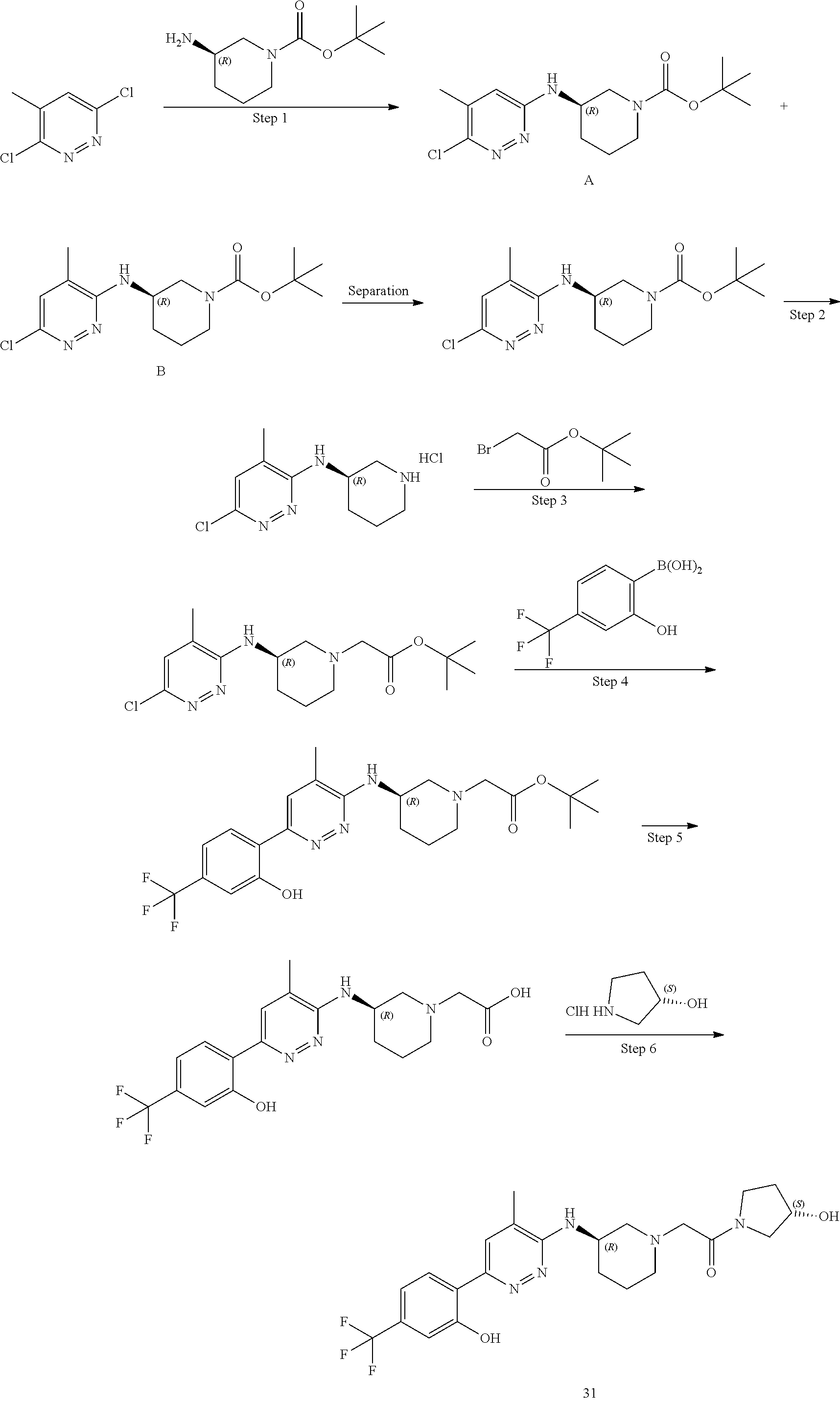

### Step 1: Synthesis of tert-butyl (R)-3-((6-chloro-4-methylpyridazin-3-yl)amino)piperidine-1-carboxylate

In a sealed tube containing 3,6-dichloro-4-methylpyridazine (5.0 g, 30.7 mmol, 1.0 eq.), tert-butyl (R)-3-aminopiperidine-1-carboxylate (6.1 g, 30.7 mmol, 1.0 eq.) and DIEA (16.0 mL, 11.9 g, 92.1 mmol, 3.0 eq.), anhydrous NMP (50 mL) was added. The reaction mixture was stirred at 150° C. for overnight in a sealed tube. After cooling down to rt, the crude reaction mixture was poured into 500 mL of water, and the solution was extracted with EtOAc (3×100 mL). All organic layers were combined, dried with anhydrous Na₂SO₄, filtered, and the filtrate was concentrated under reduced pressure using a rotary evaporator to afford the crude reaction product which was purified using a Biotage Isolera equipped with a Biotage SFAR (80 g, HC, Duo, 20 μm) and 10-33% EtOAc/Hex gradients. The combined fractions were concentrated under reduced pressure using a rotary evaporator to yield unreacted 3,6-dichloro-4-methylpyridazine (2.7 g, 55.0% recovery yield) as the first collected fraction. The desired product tert-butyl (R)-3-((6-chloro-4-methylpyridazin-3-yl)amino)piperidine-1-carboxylate (B) (595 mg, 6.0%) was the second fraction collected and was obtained as a pale yellow foamy solid. tert-Butyl (R)-3-((6-chloro-5-methylpyridazin-3-yl)amino)piperidine-1-carboxylate (A) (1.5 g, 15.0%) was the last fraction collected and was obtained as an off-white foamy solid. tert-Butyl (R)-3-((6-chloro-4-methylpyridazin-3-yl)amino)piperidine-1-carboxylate) (B): TLC: PE/EA=2:1, 254 nm, R_{f}=0.40. LC/MS: m/z=327.20 [M+H]⁺. ¹H NMR (400 MHz, Chloroform-d): δ 7.00 (s, 1H), 4.30 (d, J=7.5 Hz, 1H), 3.77-3.32 (m, 2H), 3.45 (s, 1H), 3.18-2.95 (m, 1H), 2.07 (s, 3H), 1.87 (s, 1H), 1.60-1.48 (m, 3H), 1.41 (s, 9H) ppm.

### Step 2: Synthesis of (R)-6-chloro-4-methyl-N-(piperidin-3-yl)pyridazin-3-amine hydrochloride

In a flask containing tert-butyl (R)-3-((6-chloro-4-methylpyridazin-3-yl)amino)piperidine-1-carboxylate (B) (400 mg, 1.8 mmol, 1.0 eq), the chemical was dissolved in anhydrous dioxane (4.0 mL), followed by addition of HCl (4.0 mL, 16 mmol, 4 M in 1,4-dioxane). The reaction mixture was stirred at room temperature for 1 h. The mixture was concentrated under reduced pressure using a rotary evaporator to yield (R)-6-chloro-4-methyl-N-(piperidin-3-yl)pyridazin-3-amine hydrochloride (~400 mg), as a yellow solid, was directly in used next step without further isolation and characterization. LC/MS: m/z=227.20 [M+H]⁺.

### Step 3: Synthesis of tert-butyl (R)-2-(3-((6-chloro-4-methylpyridazin-3-yl)amino)piperidin-1-yl)acetate

In a flask containing (R)-6-chloro-4-methyl-N-(piperidin-3-yl)pyridazin-3-amine hydrochloride (~400 mg, ~1.7 mmol, 1.0 eq), tert-butyl bromoacetate (261 μL, 345.1 mg, 1.7 mmol, 1.0 eq) and K₂CO₃ (488 mg, 3.5 mmol, 2.0 eq), anhydrous acetonitrile (4 mL) was added. The reaction mixture was stirred at 80° C. for overnight. After cooling down to rt, the crude reaction mixture was poured into 200 ml of water, and the solution was extracted with EtOAc (3×50 mL). All organic layers were combined, dried over anhydrous Na₂SO₄, and then concentrated under reduced pressure using a rotary evaporator to yield the crude reaction product. The crude reaction product which was on a Biotage Isolera using Biotage SFAR (12 g, HC, Duo, 20 μm) using 0-38% EA/PE gradients. The combined product containing fractions were concentrated under reduced pressure using a rotary evaporator to yield tert-butyl (R)-2-(3-((6-chloro-4-methylpyridazin-3-yl)amino)piperidin-1-yl)acetate (300.0 mg, 49.9% yield) as a pale-yellow oil. LC/MS: m/z=341.10 [M+H]⁺. ¹H NMR (400 MHz, Chloroform-d): δ 7.02-6.98 (m, 1H), 4.42 (s, 1H), 3.14-3.09 (m, 2H), 2.84-2.72 (m, 2H), 2.61-2.53 (m, 1H), 2.38 (s, 1H), 2.19-2.16 (m, 3H), 1.97 (s, 1H), 1.75 (s, 1H), 1.59-1.50 (m, 2H), 1.45 (s, 9H), 0.92-0.88 (m, 1H) ppm.

### Step 4: Synthesis of tert-butyl (R)-2-(3-((6-(2-hydroxy-4-(trifluoromethyl)phenyl)-4-methylpyridazin-3-yl)amino)piperidin-1-yl)acetate

In a flask containing tert-butyl (R)-2-(3-((6-chloro-4-methylpyridazin-3-yl)amino)piperidin-1-yl)acetate (300 mg, 0.9 mmol, 1.0 eq), (2-hydroxy-4-(trifluoromethyl)phenyl) boronic acid (278 mg, 1.3 mol, 1.5 eq), 2M Na₂CO₃ (286.2 mg, 2.7 mmol, 3.0 eq), and Pd(PPh₃)₄ (104 mg, 0.1 mol, 0.1 eq), dioxane (3 mL) was added. The reaction mixture was stirred at 110° C. overnight under an atmosphere of N₂. After cooling down to rt, the crude solution was poured into 100 mL of water, and the solution was extracted with EtOAc (3×50 mL). All organic layers were combined, dried over anhydrous Na₂SO₄, and filtered and the filtrate was then concentrated under reduced pressure using a rotary evaporator to yield the crude reaction product which was purified on a Biotage Isolera using a Biotage SFAR (4 g, HC, Duo, 20 μm) using 0-6% MeOH/DCM gradients. The compound containing fractions were combined and concentrated to yield tert-butyl (R)-2-(3-((6-(2-hydroxy-4-(trifluoromethyl)phenyl)-4-methylpyridazin-3-yl)amino)piperidin-1-yl)acetate (150.0 mg, 36.5% yield), as a pale-yellow solid. LC/MS: m/z=467.20 [M+H]⁺. ¹H NMR (400 MHz, Chloroform-d): δ 7.46-7.43 (m, 1H), 7.31 (s, 1H), 7.12 (s, 1H), 6.69 (s, 1H), 4.21 (s, 1H), 3.19-3.06 (m, 2H), 2.95-2.69 (m, 3H), 2.68 (s, 3H), 1.86 (s, 2H), 1.61 (s, 2H), 1.46 (s, 9H) ppm.

### Step 5: Synthesis of (R)-2-(3-((6-(2-hydroxy-4-(trifluoromethyl)phenyl)-4-methylpyridazin-3-yl)amino)piperidin-1-yl)acetic Acid

In a flask containing tert-butyl (R)-2-(3-((6-(2-hydroxy-4-(trifluoromethyl)phenyl)-4-methylpyridazin-3-yl)amino)piperidin-1-yl)acetate (150 mg, 0.3 mmol, 1.0 eq), the compound was dissolved in anhydrous DCM (2 mL) followed by addition of TFA (2.0 mL). The reaction mixture was stirred at rt for 1 h. The resulting mixture was concentrated under reduced pressure using a rotary evaporator to yield the crude (R)-2-(3-((6-(2-hydroxy-4-(trifluoromethyl)phenyl)-4-methylpyridazin-3-yl)amino)piperidin-1-yl)acetic acid (~300 mg) as a yellow solid that was directly in used next step without further purification. LC/MS: m/z=411.30 [M+H]⁺.

### Step 6: Synthesis of 2-((R)-3-((6-(2-Hydroxy-4-(trifluoromethyl)phenyl)-4-methylpyridazin-3-yl)amino)piperidin-1-yl)-1-((S)-3-hydroxypyrrolidin-1-yl)ethan-1-one (31)

In a flask containing (R)-2-(3-((6-(2-hydroxy-4-(trifluoromethyl)phenyl)-4-methylpyridazin-3-yl)amino)piperidin-1-yl)acetic acid (110 mg, 0.3 mmol, 1.0 eq), (S)-pyrrolidin-3-ol hydrochloride (33.1 mg, 0.3 mmol, 1.0 eq), EDCI (172.4 mg, 0.9 mmol, 3.0 eq), pyridine (2 mL) was added. The reaction mixture was stirred at 50° C. for overnight under an atmosphere of N₂. After cooling down to rt, the crude reaction mixture was concentrated under reduced pressure using a rotary evaporator to yield the crude reaction product which was purified by preparative RP-HPLC using ACN/water mixtures acidified with 0.3 vol-% FA (5-95% ACN/Water gradients in 30 min) to afford the target compound 2-((R)-3-((6-(2-hydroxy-4-(trifluoromethyl)phenyl)-4-methylpyridazin-3-yl)amino)piperidin-1-yl)-1-((S)-3-hydroxypyrrolidin-1-yl)ethan-1-one (31) (22.7 mg, 17.7% yield) as a yellow solid. LC/MS: m/z=480.45 [M+H]⁺. ¹H NMR (400 MHz, Methanol-d₄): δ 7.96 (s, 1H), 7.93 (s, 1H), 7.16 (s, 2H), 4.43-3.98 (m, 2H), 3.75-3.67 (m, 2H), 3.56 (s, 2H), 3.48 (s, 3H), 3.17 (s, 1H), 2.86 (s, 1H), 2.74 (s, 2H), 2.31 (s, 3H), 2.11-1.88 (m, 4H), 1.76 (s, 2H) ppm. ¹⁹F NMR (376 MHz, Methanol-d₄): δ −64.55 (s) ppm.

### Example 32: 2-((R)-3-((6-(2-Hydroxy-4-(trifluoromethyl)phenyl)-4-methylpyridazin-3-yl)amino)piperidin-1-yl)-1-((R)-3-hydroxypyrrolidin-1-yl)ethan-1-one (32)

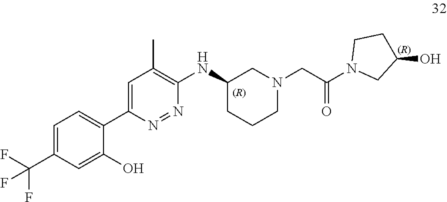

### Synthetic Scheme

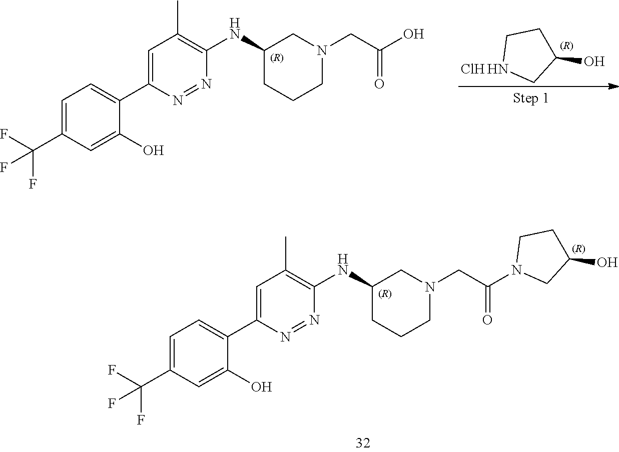

### Step 1: Synthesis of 2-((R)-3-((6-(2-hydroxy-4-(trifluoromethyl)phenyl)-4-methylpyridazin-3-yl)amino)piperidin-1-yl)-1-((R)-3-hydroxypyrrolidin-1-yl)ethan-1-one (32)

In a flask containing (R)-2-(3-((6-(2-hydroxy-4-(trifluoromethyl)phenyl)-4-methylpyridazin-3-yl)amino)piperidin-1-yl)acetic acid (prepared as disclosed in Example 10) (160 mg, 0.39 mmol, 1.0 eq), (S)-pyrrolidin-3-ol hydrochloride (48.2 mg, 0.5 mmol, 1.0 eq) and EDCI (224.1 mg, 1.2 mmol, 3.0 eq), pyridine (2.0 mL) was added. The reaction mixture was stirred at 50° C. for overnight under an atmosphere of N₂. After cooling down to rt, the crude reaction mixture was concentrated under reduced pressure using a rotary evaporator to yield crude mixture which was purified by preparative-RP-HPLC using ACN/water mixtures acidified with 0.3 vol-% FA (5-95% ACN/Water gradients in 30 min) to afford the target compound 2-((R)-3-((6-(2-hydroxy-4-(trifluoromethyl)phenyl)-4-methylpyridazin-3-yl)amino)piperidin-1-yl)-1-((R)-3-hydroxypyrrolidin-1-yl)ethan-1-one (32) (51.8 mg, 27.7% yield) as a colorless solid. LC/MS: m/z=480.20 [M+H]⁺. ¹H NMR (400 MHz, Methanol-d₄): δ 7.97 (s, 1H), 7.93 (s, 1H), 7.16 (s, 2H), 4.42-4.03 (m, 2H), 3.69 (s, 2H), 3.65 (s, 2H), 3.59-3.45 (m, 3H), 2.94 (s, 1H), 2.63 (s, 2H), 2.59 (s, 1H), 2.34-2.30 (m, 3H), 2.01 (s, 2H), 1.84 (s, 2H), 1.71 (s, 2H) ppm. ¹⁹F NMR (376 MHz, Methanol-d₄): δ −64.57 (s) ppm.

### Example 33: (R)-2-(3-((4-(2-Hydroxy-4-(trifluoromethyl)phenyl) phthalazin-1-yl)amino)piperidin-1-yl)-1-(4-hydroxypiperidin-1-yl)ethan-1-one (33)

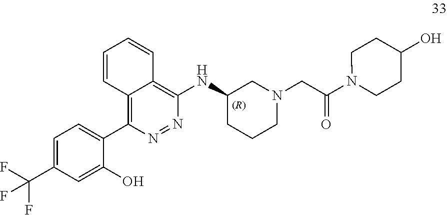

### Synthetic Scheme

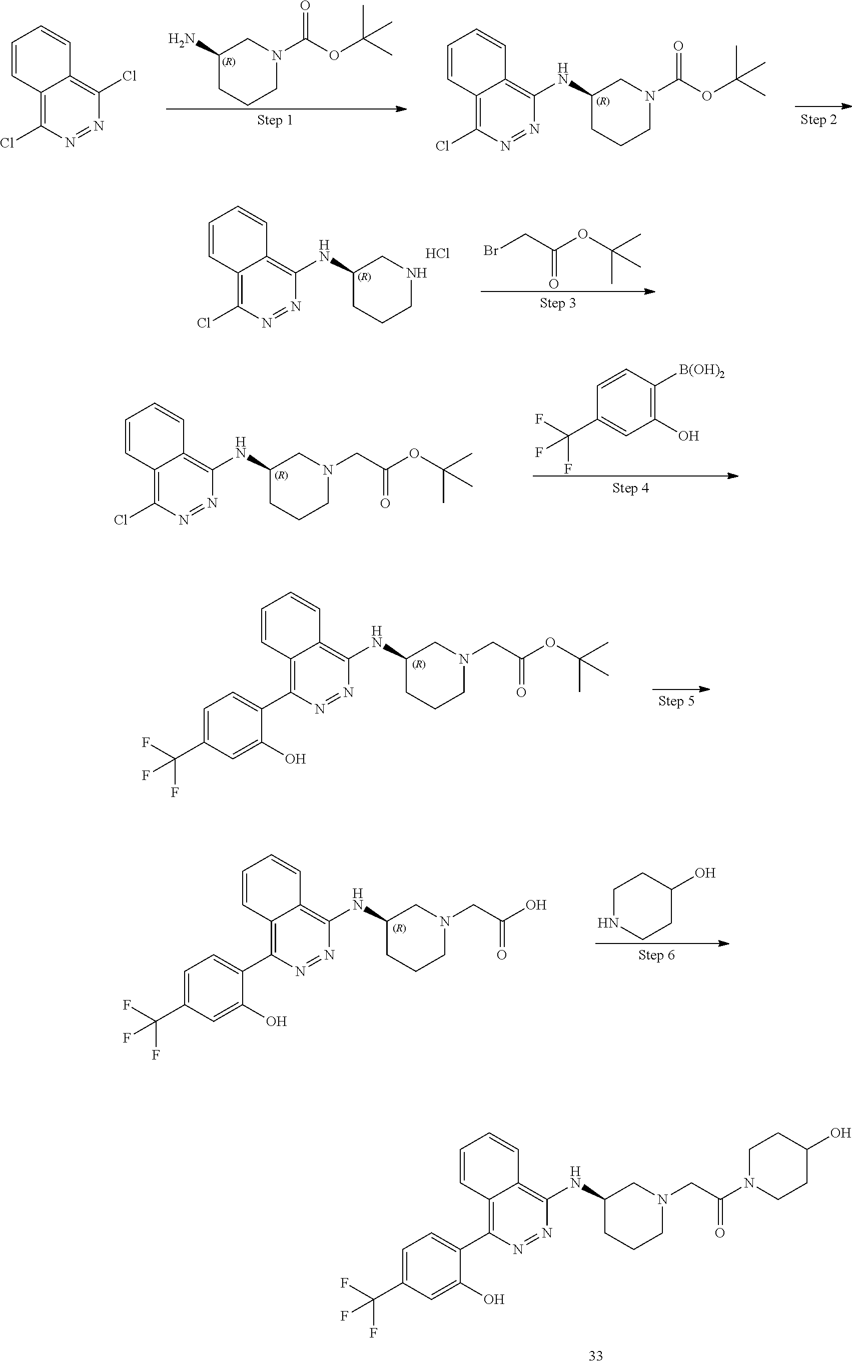

### Step 1. Synthesis of tert-butyl (R)-3-((4-chlorophthalazin-1-yl)amino)piperidine-1-carboxylate

In a sealed tube containing 1,4-dichlorophthalazine (500 mg, 2.5 mmol, 1.0 eq), tert-butyl (R)-3-aminopiperidine-1-carboxylate (503 mg, 2.5 mmol, 1.0 eq) and DIEA (1.31 mL, 974 mg, 7.5 mmol, 3.0 eq), anhydrous NMP (5.0 mL) was added. The tube was sealed and stirred at 80° C. overnight. After the reaction mixture cooled down to rt, the crude solution was poured into water (100 mL), and the solution was extracted with EtOAc (3×20 mL). The combined organic phases were washed with a 5 wt-% aqueous LiCl solution (3 x). All organic layers were combined, dried over anhydrous Na₂SO₄, filtered, and the filtrate was concentrated under reduced pressure using a rotary evaporator to yield the crude reaction product which was purified using a Biotage Isolera equipped with a Biotage SFAR (4 g, HC, Duo, 20 μM) using 0-5% MeOH/DCM. The combined product containing fractions were concentrated to afford tert-butyl (R)-3-((4-chlorophthalazin-1-yl)amino)piperidine-1-carboxylate (657 mg, 72.0% yield)) as a pale-yellow solid. LC/MS: m/z=363.10 [M+H]⁺. ¹H NMR (400 MHz, DMSO-d₆): δ 8.39 (d, J=8.3 Hz, 1H), 7.86 (t, J=7.6 Hz, 1H), 7.79 (t, J=7.9 Hz, 1H), 7.47 (d, J=8.1 Hz, 1H), 4.55 (s, 1H), 3.50 (s, 2H), 3.39-3.15 (m, 1H), 3.08-2.95 (m, 1H), 2.64-2.25 (m, 2H), 1.96 (s, 1H), 1.80 (s, 1H), 1.73 (d, J=8.3 Hz, 1H), 1.66-1.53 (m, 1H), 1.42 (s, 9H) ppm.

### Steps 2-5: Synthesis of (R)-2-(3-((4-(2-hydroxy-4-(trifluoromethyl)phenyl) phthalazin-1-yl)amino)piperidin-1-yl)acetic Acid

(R)-2-(3-((4-(2-hydroxy-4-(trifluoromethyl)phenyl) phthalazin-1-yl)amino)piperidin-1-yl)acetic acid was prepared from tert-butyl (R)-3-((4-chlorophthalazin-1-yl)amino)piperidine-1-carboxylate in an identical 4 step sequence to (R)-2-(3-((6-(2-hydroxy-4-(trifluoromethyl)phenyl)-4-methylpyridazin-3-yl)amino)piperidin-1-yl)acetic acid (described above), isolated as a yellow solid, and directly used next step. LC/MS: m/z=447.20 [M+H]⁺.

### Step 6: Synthesis of (R)-2-(3-((4-(2-hydroxy-4-(trifluoromethyl)phenyl) phthalazin-1-yl)amino)piperidin-1-yl)-1-(4-hydroxypiperidin-1-yl)ethan-1-one (33)

In a flask with (R)-2-(3-((4-(2-hydroxy-4-(trifluoromethyl)phenyl) phthalazin-1-yl)amino)piperidin-1-yl)acetic acid (160.0 mg, 0.4 mmol, 1.0 eq), 4-hydroxypiperidine (40.0 mg, 0.4 mmol, 1.0 eq) and EDCI (230.0 mg, 1.2 mmol, 3.0 eq), pyridine (2.0 mL) was added. The reaction mixture was stirred at 50° C. for overnight under an atmosphere of N₂. After cooling down to rt, the crude solution was concentrated under reduced pressure using a rotary evaporator to yield the crude reaction mixture. The crude reaction mixture was purified on preparative RP-HPLC using ACN/water gradients acidified with 0.3 vol-% FA to afford the target compound (R)-2-(3-((4-(2-hydroxy-4-(trifluoromethyl)phenyl) phthalazin-1-yl)amino)piperidin-1-yl)-1-(4-hydroxypiperidin-1-yl)ethan-1-one (33) formate salt (29.0 mg, 15.3% yield) after lyophilization of the combined product containing fractions as an off-white solid. LC/MS: 530.25 [M+H]⁺. ¹H NMR (400 MHz, Methanol-d₄): δ 8.39 (s, 1H), 8.35 (s, 1H), 7.90 (s, 1H), 7.82 (s, 1H), 7.61 (s, 1H), 7.52 (s, 1H), 7.30 (s, 1H), 7.25 (s, 1H), 4.58 (s, 1H), 4.00 (s, 1H), 3.71 (s, 3H), 3.52 (s, 1H), 3.40 (s, 1H), 3.18 (s, 2H), 3.04 (s, 2H), 2.74 (s, 1H), 2.58-2.35 (m, 1H), 2.08 (s, 1H), 1.84 (s, 6H), 1.44 (d, J=3.8 Hz, 2H) ppm. ¹⁹F NMR (376 MHz, Methanol-d₄): δ −64.34 (s) ppm.

### Example 34: (R)-2-(3-((4-(2-Hydroxy-4-fluorophenyl) phthalazin-1-yl)amino)piperidin-1-yl)-1-(4-hydroxypiperidin-1-yl)ethan-1-one (34)

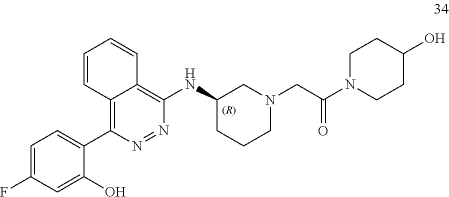

### Synthetic Scheme

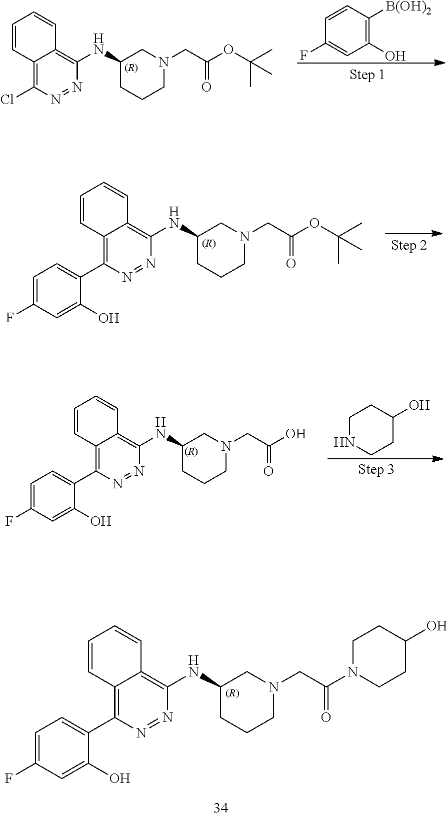

### Steps 1-2: Synthesis of (R)-2-(3-((4-(4-fluoro-2-hydroxyphenyl)phthalazin-1-yl)amino)piperidin-1-yl)acetic Acid

(R)-2-(3-((4-(4-fluoro-2-hydroxyphenyl) phthalazin-1-yl)amino)piperidin-1-yl)acetic acid was prepared in the same manner as described herein for (R)-2-(3-((4-(2-hydroxy-4-(trifluoromethyl)phenyl) phthalazin-1-yl)amino)piperidin-1-yl)acetic acid (Example 33), except using (4-fluoro-2-hydroxyphenyl) boronic acid as a starting material to afford (196.0 mg, 52.5% yield) of the isolated material as a pale-yellow solid. LC/MS: (ESI) calcd C₂₁H₂₁FN₄O₃: 396.16; found: m/z=397.15 [M+H]⁺. ¹H NMR (400 MHz, DMSO-d₆): δ 8.39 (d, J=8.3 Hz, 1H), 7.86 (t, J=7.6 Hz, 1H), 7.79 (t, J=7.9 Hz, 1H), 7.47 (d, J=8.1 Hz, 1H), 7.28 (t, J=7.7 Hz, 1H), 6.77-6.42 (m, 2H), 4.55 (s, 1H), 3.50 (s, 2H), 3.39-3.28 (m, 1H), 3.08-2.85 (m, 1H), 2.64-2.35 (m, 2H), 1.96 (s, 1H), 1.80 (s, 1H), 1.73 (d, J=8.3 Hz, 1H), 1.66-1.53 (m, 1H) ppm. ¹⁹F NMR (376 MHz, DMSO-d₆): δ −110.52-−110.61 (m) ppm.

### Step 3: Synthesis of (R)-2-(3-((4-(2-hydroxy-4-fluorophenyl) phthalazin-1-yl)amino)piperidin-1-yl)-1-(4-hydroxypiperidin-1-yl)ethan-1-one (34)

In a flask containing (R)-2-(3-((4-(4-fluoro-2-hydroxyphenyl) phthalazin-1-yl)amino)piperidin-1-yl)acetic acid (90.0 mg, 0.2 mmol, 1.0 eq.), 4-hydroxypiperidine (48.0 mg, 0.5 mmol, 1.0 eq.) and EDCI (276.0 mg, 1.4 mmol, 3.0 eq.), pyridine (2.0 mL) was added. The reaction mixture was stirred at 50° C. for overnight under an atmosphere of N₂. After cooling down to rt, the crude solution was concentrated under reduced pressure using a rotary evaporator to afford the crude reaction mixture. The crude reaction product was purified using a preparative RP-HPLC using ACN/water mixtures acidified with 0.3 vol-% FA (5-95% ACN/Water gradients in 30 min) to afford the target compound (R)-2-(3-((4-(2-hydroxy-4-fluorophenyl) phthalazin-1-yl)amino)piperidin-1-yl)-1-(4-hydroxypiperidin-1-yl)ethan-1-one (34) (81.6 mg, 35.5% yield) after lyophilization of the product containing fractions as a pale-yellow solid. LC/MS: (ESI) calcd C₂₆H₃₀FN₅O₃: 479.23; found: m/z=480.20 [M+H]⁺. ¹H NMR (400 MHz, DMSO-d₆): δ 8.38-8.30 (m, 1H), 7.82 (d, J=9.7 Hz, 1H), 7.74 (s, 1H), 7.56-7.49 (m, 1H), 7.18 (s, 2H), 7.03 (d, J=6.3 Hz, 1H), 6.59 (s, 2H), 4.38 (s, 1H), 4.06 (d, J=26.1 Hz, 1H), 3.85 (s, 1H), 3.71 (s, 1H), 3.54 (s, 1H), 3.43 (s, 2H), 3.17 (s, 2H), 3.06 (s, 1H), 3.08-2.95 (m, 1H), 2.80 (s, 2H), 2.01 (s, 4H), 1.78 (s, 2H), 1.63-1.38 (m, 4H), 1.23 (s, 1H) ppm. ¹⁹F NMR (376 MHz, DMSO-d₆): δ −117.74-−112.96 (m) ppm.

### Example 35: (S)-1-(2-((R)-3-((4-(2-Hydroxy-4-(trifluoromethyl)phenyl)-5,7-dihydrofuro[3,4-d]pyridazin-1-yl)amino)piperidin-1-yl)ethyl)pyrrolidin-3-ol (35)

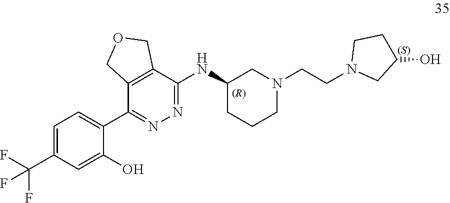

### Synthetic Scheme:

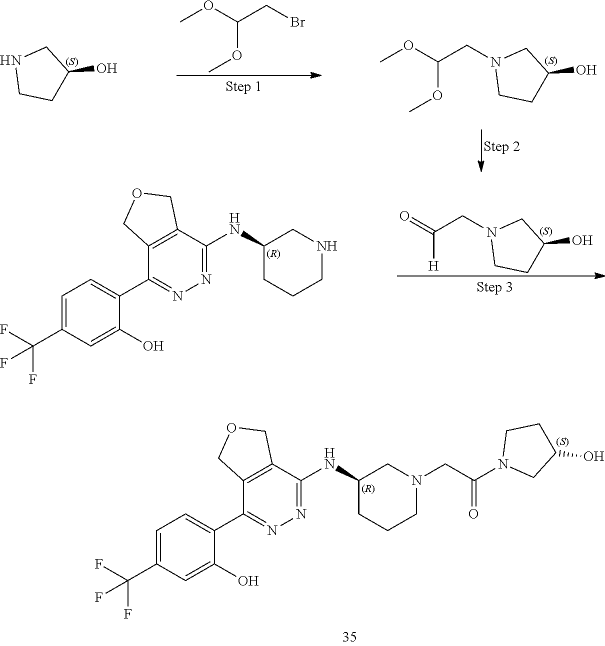

### Step 1: Synthesis of (S)-1-(2,2-dimethoxyethyl)pyrrolidin-3-ol

To a solution of (S)-pyrrolidin-3-ol (500 mg, 5.75 mmol, 1.0 eq.) in 1,4-dioxane (10 mL) were added 2-bromo-1,1-dimethoxyethane (1.05 mL, 1.5 g, 8.62 mmol, 1.5 eq.) and K₂CO₃ (2.40 g, 17.24, 3.0 eq. mmol). The resulting reaction mixture was stirred at 100° C. for 24 h. After filtration and concentration under reduced pressure using a rotary evaporator, the crude product of (S)-1-(2,2-dimethoxyethyl)pyrrolidin-3-ol (500 mg) was obtained, which was used directly without any further purification and characterization.

### Step 2: Synthesis of (S)-2-(3-hydroxypyrrolidin-1-yl)acetaldehyde

(S)-1-(2,2-dimethoxyethyl)pyrrolidin-3-ol (500 mg crude) was dissolved in 5 mL of 6 M hydrochloric acid (HCl (aq.))). After stirring for 24 h at 100° C., the suspension was concentrated under reduced pressure using a rotary evaporator to afford(S)-2-(3-hydroxypyrrolidin-1-yl)acetaldehyde (550 mg), which was used directly without any further purification and characterization.

### Step 3: Synthesis of (S)-1-(2-((R)-3-((4-(2-hydroxy-4-(trifluoromethyl)phenyl)-5,7-dihydrofuro[3,4-d]pyridazin-1-yl)amino)piperidin-1-yl)ethyl)pyrrolidin-3-ol (35)

To a light yellow solution of (R)-2-(4-(piperidin-3-ylamino)-5,7-dihydrofuro[3,4-d]pyridazin-1-yl)-5-(trifluoromethyl)phenol (150 mg, 0.36 mmol, 1.0 eq.) in MeOH (5 mL) were added(S)-2-(3-hydroxypyrrolidin-1-yl)acetaldehyde (186 mg, 1.44 mmol, 4.0 eq.) and NaBH₃CN (68 mg, 1.08 mmol, 3.0 eq.). After stirring for 4 h at 70° C., the suspension was concentrated under reduced pressure using a rotary evaporator. The residue was purified by reverse flash chromatography with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 45% B in 10 min, 45% B; Wavelengths: 254/220 nm; RT1: 8.9 min. After lyophilization of the combined product containing fractions, the target compound(S)-1-(2-((R)-3-((4-(2-hydroxy-4-(trifluoromethyl)phenyl)-5,7-dihydrofuro[3,4-d]pyridazin-1-yl)amino)piperidin-1-yl)ethyl)pyrrolidin-3-ol (35) was obtained as light yellow solid (27.9 mg, 15.8% yield). LC/MS: MS (ESI) calcd for C₂₄H₃₀F₃N₅O₃: 493.23; found: m/z=494.20 [M+H]⁺. ¹H-NMR (400 MHz, Methanol-d₄): δ 7.42-7.32 (m, 1H), 7.21-7.14 (m, 2H), 5.35 (s, 2H), 5.03 (s, 2H), 4.44-4.31 (m, 2H), 3.20-3.08 (m, 1H), 2.94-2.52 (m, 9H), 2.36-1.93 (m, 4H), 1.91-1.41 (m, 4H) ppm. ¹⁹F NMR (376 MHz, Methanol-d₄): δ −64.44 ppm.

### Example 36: (R)-1-(2-((R)-3-((4-(2-Hydroxy-4-(trifluoromethyl)phenyl)-5,7-dihydrofuro[3,4-d]pyridazin-1-yl)amino)piperidin-1-yl)ethyl)pyrrolidin-3-ol (36)

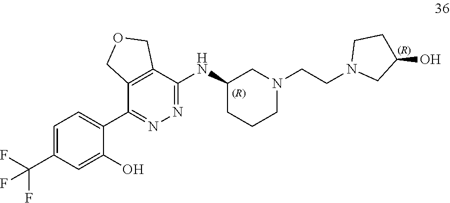

### Synthetic Scheme:

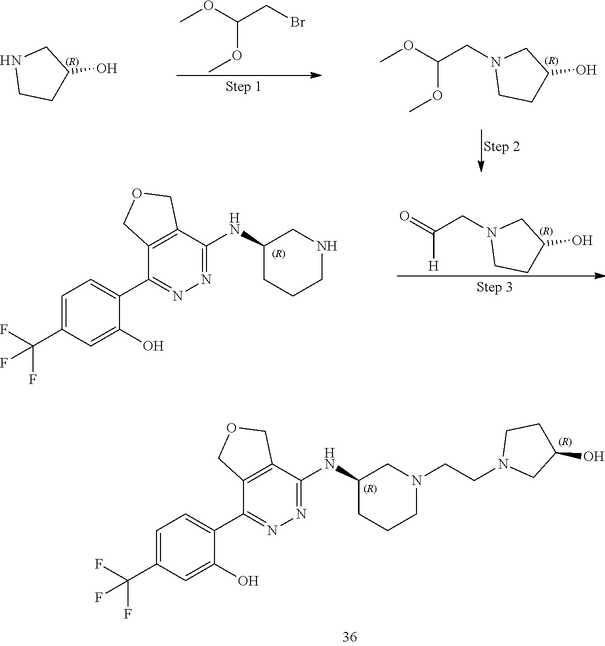

### Step 1: Synthesis of (R)-1-(2,2-dimethoxyethyl)pyrrolidin-3-ol (36)

To a solution of (R)-pyrrolidin-3-ol (500 mg, 5.75 mmol, 1.0 eq.) in 1,4-dioxane (10 mL) were added 2-bromo-1,1-dimethoxyethane (1.05 mL, 1.50 g, 8.62 mmol, 1.5 eq.) and K₂CO₃ (2.40 g, 17.2 mmol, 3.0 eq.). The resulting reaction mixture was stirred at 100° C. for 24 h. After filtration and concentration of the filtrate under reduced pressure using a rotary evaporator, the crude product of (R)-1-(2,2-dimethoxyethyl)pyrrolidin-3-ol (500 mg) was obtained, which was used directly without any further purification and characterization.

### Step 2: Synthesis of (R)-2-(3-hydroxypyrrolidin-1-yl)acetaldehyde

(R)-1-(2,2-dimethoxyethyl)pyrrolidin-3-ol (500 mg crude) was dissolved in 5 mL of 6 M hydrochloric acid (HCl (aq.). After stirring for 24 h at 100° C., the suspension was concentrated under reduced pressure using a rotary evaporator, obtaining crude (R)-2-(3-hydroxypyrrolidin-1-yl)acetaldehyde (550 mg), which was used directly without any further purification and characterization.

### Step 3: Synthesis of (R)-1-(2-((R)-3-((4-(2-Hydroxy-4-(trifluoromethyl)phenyl)-5,7-dihydrofuro[3,4-d]pyridazin-1-yl)amino)piperidin-1-yl)ethyl)pyrrolidin-3-ol (36)

To a light yellow solution of (R)-2-(4-(piperidin-3-ylamino)-5,7-dihydrofuro[3,4-d]pyridazin-1-yl)-5-(trifluoromethyl)phenol (100 mg, 0.24 mmol, 1.0 eq.) in MeOH (2 mL) were added crude (R)-2-(3-hydroxypyrrolidin-1-yl)acetaldehyde (~123 mg, ~0.96 mmol, ~4.0 eq.) and NaBH₃CN (45 mg, 0.72 mmol, 3.0 eq.). After stirring for 4 h at 70° C., the suspension was concentrated under reduced pressure using a rotary evaporator. The residue was purified by reverse phase flash chromatography using the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 5% B to 95% B in 7 min, 95% B; Wavelength: 254 nm; RT1: 5.42 min; Injection Volume: 1.2 mL; Number of Runs: 2. After lyophilization of the combined product containing fractions, the target compound (R)-1-(2-((R)-3-((4-(2-hydroxy-4-(trifluoromethyl)phenyl)-5,7-dihydrofuro[3,4-d]pyridazin-1-yl)amino)piperidin-1-yl)ethyl)pyrrolidin-3-ol (36) was obtained as a light yellow solid (13.0 mg, 11.0% yield). LC/MS: MS (ESI) calcd for C₂₄H₃₀F₃N₅O₃: 493.23, found: m/z=494.25 [M+H]⁺. ¹H NMR (400 MHz, Methanol-d₄): δ 7.39-7.29 (m, 1H), 7.23-7.10 (m, 2H), 5.35 (s, 2H), 5.03 (s, 2H), 4.44-4.29 (m, 2H), 3.21-3.09 (m, 1H), 2.95-2.87 (m, 1H), 2.87-2.70 (m, 4H), 2.70-2.53 (m, 4H), 2.34-2.23 (m, 1H), 2.23-2.08 (m, 2H), 2.08-1.96 (m, 1H), 1.89-1.79 (m, 1H), 1.79-1.67 (m, 2H), 1.60-1.45 (m, 1H) ppm. ¹⁹F NMR (376 MHz, Methanol-d₄): δ −64.44 ppm.

### Example 37: (R)-1-(2-(3-((6-(2-Hydroxy-4-(trifluoromethyl)phenyl)-5-methylpyridazin-3-yl)amino)piperidin-1-yl)ethyl)azetidin-3-ol (37)

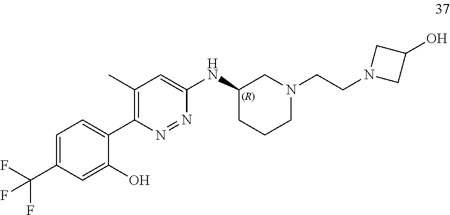

### Synthetic Scheme:

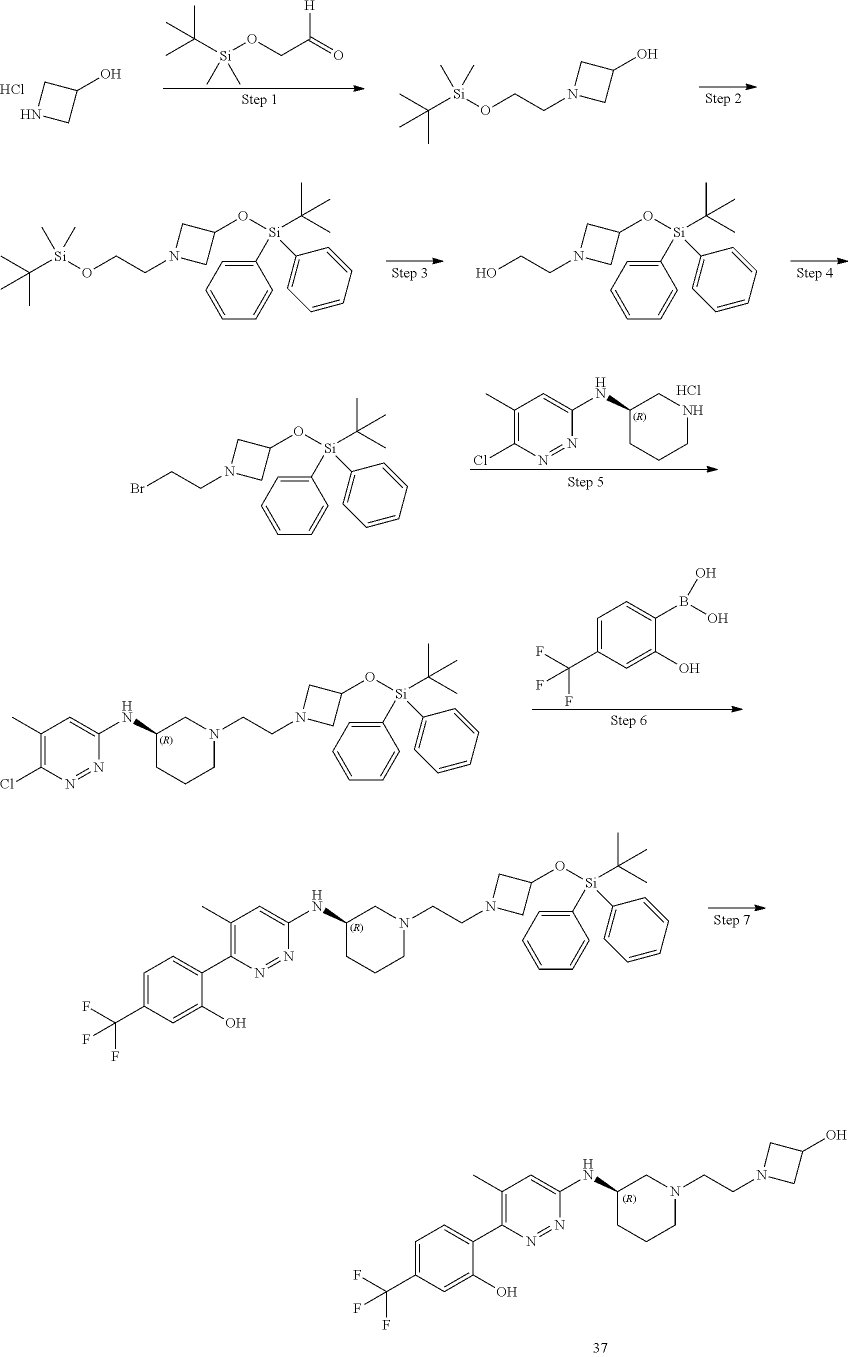

### Step 1: Synthesis of 1-(2-((tert-butyldimethylsilyl)oxy)ethyl)azetidin-3-ol

A colorless suspension of 2-[(tert-butyldimethylsilyl)oxy]acetaldehyde (2.5 mL, 2.29 g, 13.12 mmol) and azetidine-ol-hydrochloride (1.57 g, 14.4 mmol, 1.1 eq.) in MeOH (20 mL) was stirred at rt for 0.5 h. Then, to this suspension NaBH₃CN (1.24 g, 19.68 mmol, 1.5 eq.) was added leading to evolution of gas and a colorless solution, which was stirred at rt for 2 h. The reaction was quenched by the addition of water (50 mL). The obtained mixture was extracted with EtOAc (3×50 mL). The combined organic phases were washed with brine (2×50 mL), dried over Na₂SO₄, filtered and the filtrate was evaporated under reduced pressure using a rotary evaporator to afford 1-{2-[(tert-butyldimethylsilyl)oxy]ethyl}azetidin-3-ol as a yellow oil (2.4 g, 79.0% yield). LC/MS: mass calcd for C₁₁H₂₅NO₂Si: 231.17, found: m/z=232.05 [M+H]⁺.

### Step 2: Synthesis of 1-(2-((tert-butyldimethylsilyl)oxy)ethyl)-3-((tert-butyldiphenylsilyl)oxy)azetidine

A colorless suspension of 1-{2-[(tert-butyldimethylsilyl)oxy]ethyl}azetidin-3-ol (2.4 g, 10.4 mmol 1.0 eq.), TBDPSCl (3.42 g, 12.5 mmol, 1.2 eq.) and 1H-imidazole (2.12 g, 31.1 mmol, 3.0 eq.) in DCM (25 mL) was stirred at rt for overnight. The reaction was diluted with DCM (50 mL) and the organic phase was washed with water (3×50 mL) and brine (50 mL). The combined organic phases were concentrated under reduced pressure using a rotary evaporator and purified by silica gel column chromatography (0~80% Et₂O/PE) to afford 1-{2-[(tert-butyldimethylsilyl)oxy]ethyl}-3-[(tert-butyldiphenylsilyl)oxy]azetidine as a light yellow oil (2.5 g, 47.3% yield). LC/MS: mass calcd for C₂₇H₄₃NO₂Si₂: 469.28; found: m/z=470.35 [M+H]⁺.

### Step 3: Synthesis of 2-(3-((tert-butyldiphenylsilyl)oxy)azetidin-1-yl)ethan-1-ol

A yellow solution of 1-{2-[(tert-butyldimethylsilyl)oxy]ethyl}-3-[(tert-butyldiphenylsilyl)oxy]azetidine (2.38 g, 5.07 mmol) and sodium tetrachloroaurate (III) dihydrate (1.01 g, 2.53 mmol) in DCM (4.8 mL) was stirred at 80° C. for 2 h. After concentration under reduced pressure using a rotary evaporator, the obtained residue was purified by silica gel column chromatography to afford 2-{3-[(tert-butyldiphenylsilyl)oxy]azetidin-1-yl}ethanol as a yellow oil (1.25 g, 54.9% yield). LC/MS: mass calcd for C₂₁H₂₉NO₂Si: 355.20; found: m/z=356.10 [M+H]⁺.

### Step 4: Synthesis of 1-(2-bromoethyl)-3-((tert-butyldiphenylsilyl)oxy)azetidine

A colorless solution of PPh₃ (1.62 g, 6.19 mmol, 2.0 eq.) in THF (2 mL) was added dropwise into a solution of 2-{3-[(tert-butyldiphenylsilyl)oxy]azetidine-1-yl}ethanol (1.1 g, 3.09 mmol, 1.0 eq.) and NBS (1.10 g, 6.19 mmol, 2.0 eq.) in THF (18 mL) at 0° C. under an atmosphere of N₂ to yield a solution which was allowed to warm to rt and stirred at rt for 4 h. The reaction solution was subjected to reverse phase column chromatography purification eluting with PE/Et₂O gradients (0~100%) giving 1-(2-bromoethyl)-3-[(tert-butyldiphenylsilyl)oxy]azetidine as a colorless solid (500 mg, 21.8% yield). LC/MS: mass calcd for C₂₁H₂₈BrNOSi: 417.11, found: m/z=418.15 [M+H]⁺.

### Step 5: Synthesis of (R)—N-(1-(2-(3-((tert-butyldiphenylsilyl)oxy)azetidin-1-yl)ethyl)piperidin-3-yl)-6-chloro-5-methylpyridazin-3-amine

A yellow suspension of 1-(2-bromoethyl)-3-[(tert-butyldiphenylsilyl)oxy]azetidine (420 mg, 1.00 mmol, 1.1 eq.), 6-chloro-5-methyl-N-[(3R)-piperidin-3-yl]pyridazin-3-amine hydrochloride (240 mg, 0.91 mmol, 1.0 eq.), NaI (205 mg, 1.37 mmol, 1.5 eq.) and DIEA (636 μL, 472 mg, 3.65 mmol, 4.0 eq.) in ACN (5 mL) was stirred at 80° C. for 2 h. The reaction mixture was diluted with EtOAc (20 mL) and washed with water (3×20 mL) and brine (20 mL). The organic phase was concentrated under reduced pressure using a rotary evaporator and the crude product was subjected to silica gel column chromatography purification eluting with PE/EA gradients (0~50%) to afford N-[(3R)-1-(2-{3-[(tert-butyldiphenylsilyl)oxy]azetidin-1-yl}ethyl)piperidin-3-yl]-6-chloro-5-methylpyridazin-3-amine as a light yellow oil (150 mg, 29.2% yield). LC/MS: mass calcd for C₃₁H₄₂ClN₅OSi: 563.28, found: m/z=564.40 [M+H]⁺.

### Step 6: Synthesis of (R)-2-(6-((1-(2-(3-((tert-butyldiphenylsilyl)oxy)azetidin-1-yl)ethyl)piperidin-3-yl)amino)-4-methylpyridazin-3-yl)-5-(trifluoromethyl)phenol

A brown solution of N-[(3R)-1-(2-{3-[(tert-butyldiphenylsilyl)oxy]azetidin-1-yl}ethyl)piperidin-3-yl]-6-chloro-5-methylpyridazin-3-amine (140 mg, 0.25 mmol), 2-hydroxy-4-(trifluoromethyl)phenylboronic acid (77 mg, 0.372 mmol, 1.5 eq.), Pd(PPh₃)₄ (9 mg, 0.007 mmol, 0.03 eq.), NaHCO₃ (63 mg, 0.74 mmol, 3.0 eq.) in dioxane (3 mL) and water (1.5 mL) was stirred in a sealed tube at 100° C. under an atmosphere of N₂ for 2 h. The solvents were evaporated under reduced pressure using a rotary evaporator and the residue was further purified by silica gel chromatography eluting with DCM/MeOH gradients (0~10%) to afford 1-{2-[(3R)-3-({6-[2-hydroxy-4-(trifluoromethyl)phenyl]-5-methylpyridazin-3-yl}amino)piperidin-1-yl]ethyl}azetidin-3-ol as a light brown oil (80 mg, 69.9% yield). LC/MS: mass calcd for C₂₂H₂₈F₃N₅O₂: 689.34, found: m/z=690.45 [M+H]⁺.

### Step 7: Synthesis of (R)-1-(2-(3-((6-(2-hydroxy-4-(trifluoromethyl)phenyl)-5-methylpyridazin-3-yl)amino)piperidin-1-yl)ethyl)azetidin-3-ol (37)

A yellow solution of 2-(6-{[(3R)-1-(2-{3-[(tert-butyldiphenylsilyl)oxy]azetidin-1-yl}ethyl)piperidin-3-yl]amino}-4-methylpyridazin-3-yl)-5-(trifluoromethyl)phenol (60 mg, 0.09 mmol) and NaOH (35 mg, 0.87 mmol) in MeOH (2 mL) was stirred at 80° C. for 4 h. The reaction mixture was further purified by preparative RP-HPLC using the following conditions: Column: XSelect CSH Prep C18 OBD Column, 19*250 mm, 5 μm; Mobile Phase A: Water (10 mmol/L NH₄HCO₃), Mobile Phase B: ACN; Flow rate: 25 mL/min; Gradient: 30% B to 60% B in 7 min, 60% B; Wavelength: 254 nm; RT1: 6.32 min. After lyophilization of the combined product containing fractions, the target compound 1-{2-[(3R)-3-({6-[2-hydroxy-4-(trifluoromethyl)phenyl]-5-methylpyridazin-3-yl}amino)piperidin-1-yl]ethyl}azetidin-3-ol (37) was obtained as a colorless solid (12.6 mg, 31.9% yield). LC/MS: mass calcd for C₂₂H₂₈F₃N₅O₂: 451.22, found: m/z=452.30 [M+H]⁺. ¹H NMR (400 MHz, DMSO-d₆): δ 7.39-7.33 (m, 1H), 7.21-7.14 (m, 2H), 6.68 (s, 1H), 4.16-4.08 (m, 1H), 4.01-3.92 (m, 1H), 3.53-3.44 (m, 2H), 2.97-2.88 (m, 1H), 2.72-2.59 (m, 3H), 2.48-2.45 (m, 2H), 2.29-2.19 (m, 2H), 2.10-1.97 (m, 4H), 1.97-1.78 (m, 2H), 1.74-1.63 (m, 1H), 1.58-1.45 (m, 1H), 1.35-1.20 (m, 1H) ppm. ¹⁹F NMR (376 MHz, DMSO-d₆): δ −61.13 ppm.

### Example 38: 2-(4-Methyl-6-{[(3R)-1′-[(1s,3s)-3-hydroxycyclobutyl]-[1,4′-bipiperidin]-3-yl]amino}pyridazin-3-yl)-5-(trifluoromethyl)phenol (38a) and 2-(4-Methyl-6-{[(3R)-1′-[(1r,3r)-3-hydroxycyclobutyl]-[1,4′-bipiperidin]-3-yl]amino}pyridazin-3-yl)-5-(trifluoromethyl)phenol (38b)

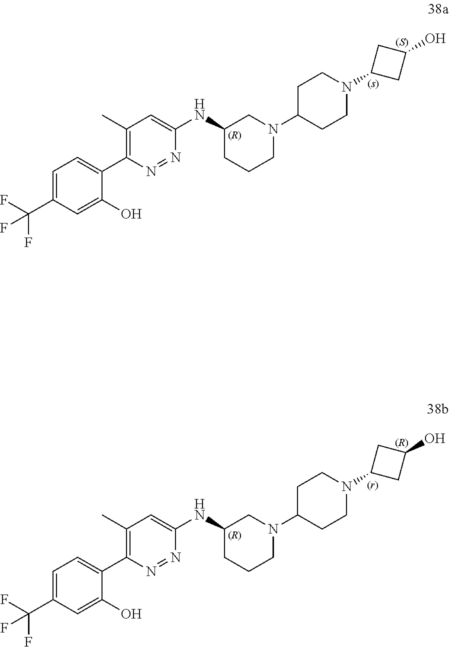

### Synthetic Scheme:

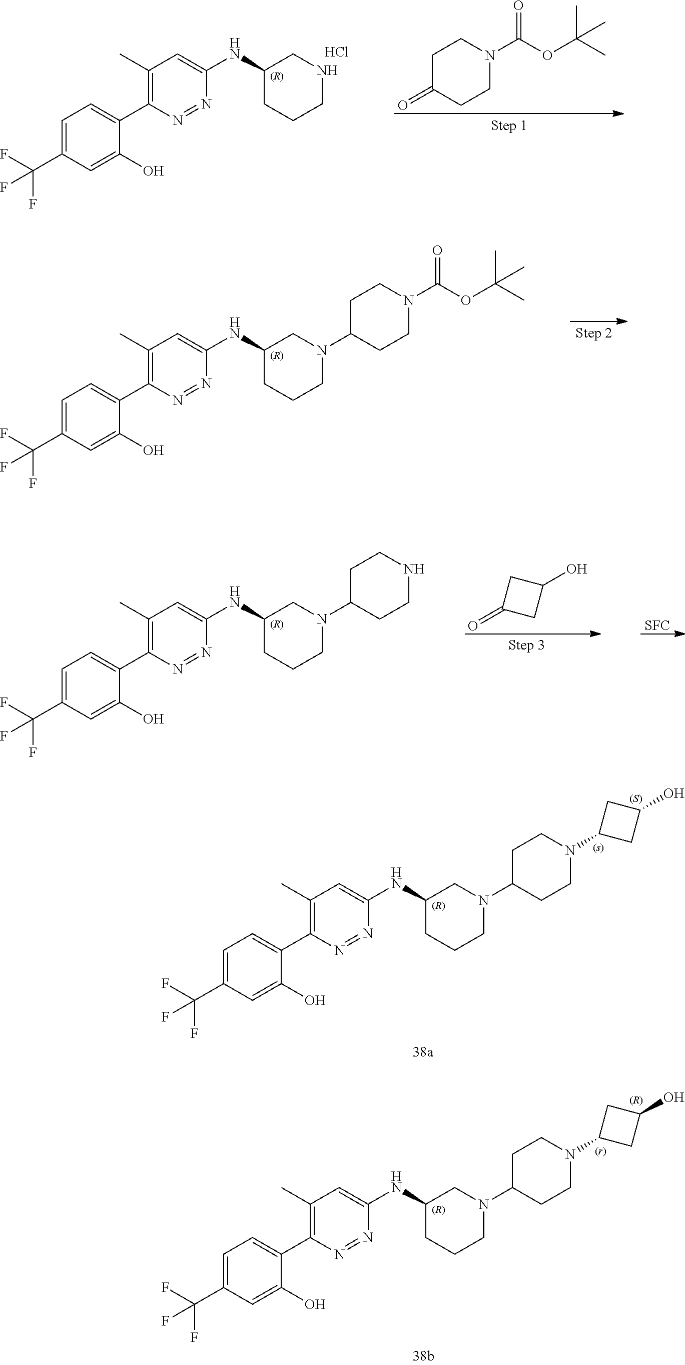

### Step 1: Synthesis of tert-butyl (3R)-3-({6-[2-hydroxy-4-(trifluoromethyl)phenyl]-5-methylpyridazin-3-yl}amino)-[1,4′-bipiperidine]-1′-carboxylate

To a light yellow solution of 2-{4-methyl-6-[(3R)-piperidin-3-ylamino]pyridazin-3-yl}-5-(trifluoromethyl)phenol hydrochloride (1.60 g, 4.12 mmol, 1.0 eq.) in MeOH (20 mL) were added tert-butyl 4-oxopiperidine-1-carboxylate (900 mg, 4.53 mmol, 1.1 eq.) and NaBH₃CN (780 mg, 12.35 mmol, 3.0 eq.). After stirring for 2 h at rt, the suspension was concentrated under reduced pressure using a rotary evaporator. The residue was purified by reverse phase flash chromatography using the following conditions: column, C18 silica gel; mobile phase, ACN in water, 10% to 60% gradient in 20 min; detector, UV 254 nm to afford tert-butyl (3R)-3-({6-[2-hydroxy-4-(trifluoromethyl)phenyl]-5-methylpyridazin-3-yl}amino)-[1,4′-bipiperidine]-1′-carboxylate (1.80 g, 81.7% yield) as light yellow solid. LC/MS: MS (ESI) calcd for C₂₇H₃₆F₃N₅O₃: 535.28, found: m/z=536.85 [M+H]⁺.

### Step 2: Synthesis of 2-{6-[(3R)-[1,4′-Bipiperidin]-3-ylamino]-4-methylpyridazin-3-yl}-5-(trifluoromethyl)phenol

To a light yellow solution of tert-butyl (3R)-3-({6-[2-hydroxy-4-(trifluoromethyl)phenyl]-5-methylpyridazin-3-yl}amino)-[1,4′-bipiperidine]-1′-carboxylate (1.80 g, 3.36 mmol, 1 eq.) in DCM (5 mL) was added a solution of TFA (2 mL) in DCM (5 mL). After stirring for 30 min at rt, the suspension was concentrated under reduced pressure using a rotary evaporator. The residue was purified by reverse flash chromatography using the following conditions: Column, C18 silica gel; mobile phase, ACN in water, 10% to 60% gradient in 20 min; detector, UV 254 nm to afford 2-{6-[(3R)-[1,4′-bipiperidin]-3-ylamino]-4-methylpyridazin-3-yl}-5-(trifluoromethyl)phenol (1.30 g, 88.8% yield) as light yellow solid. LC/MS: MS (ESI) calcd for C₂₂H₂₈F₃N₅O: 435.22; found: m/z=436.25 [M+H]⁺.

### Step 3: Synthesis of 2-(4-Methyl-6-{[(3R)-1′-[(1s,3s)-3-hydroxycyclobutyl]-[1,4′-bipiperidin]-3-yl]amino}pyridazin-3-yl)-5-(trifluoromethyl)phenol (38a) and 2-(4-methyl-6-{[(3R)-1′-[(1r,3r)-3-hydroxycyclobutyl]-[1,4′-bipiperidin]-3-yl]amino}pyridazin-3-yl)-5-(trifluoromethyl)phenol (38b)

To a light yellow solution of 2-{6-[(3R)-[1,4′-bipiperidin]-3-ylamino]-4-methylpyridazin-3-yl}-5-(trifluoromethyl)phenol (200 mg, 0.46 mmol, 1.0 eq.) in MeOH (3 mL) were added 3-hydroxycyclobutan-1-one (50 mg, 0.55 mmol, 1.2 eq.) and NaBH₃CN (90 g, 1.38 mmol, 3.0 eq.). After stirring for 2 h at rt, the suspension was concentrated under reduced pressure using a rotary evaporator. The residue was purified by reverse flash chromatography with the following conditions: Column: XBridge Prep Phenyl OBD Column, 19*100 mm, 5 μm; Mobile Phase A: Water (10 mmol/L NH₄HCO₃), Mobile Phase B: ACN; Flow rate: 60 mL/min; Gradient: 23% B to 53% B in 7 min, 53% B; Wavelength: 254 nm; RT1: 6-6.5 min, and the product was further purified by SFC using the following condition: Column: Lux 5 μm Cellulose-4, 2.12*25 cm, 5 μm; Mobile Phase A: Hex (0.5 vol-% 2M NH₃-MeOH)-HPLC, Mobile Phase B: IPA-HPLC; Flow rate: 20 mL/min; Gradient: 35% B to 35% B in 19 min; Wavelengths: 220/254 nm; Sample solvent: EtOH-HPLC; Injection Volume: 0.6 mL; with RT1: 9.75 min; affording a single stereoisomer (randomly assigned “cis”) 2-(4-methyl-6-{[(3R)-1′-[(1s,3s)-3-hydroxycyclobutyl]-[1,4′-bipiperidin]-3-yl]amino}pyridazin-3-yl)-5-(trifluoromethyl)phenol (38a) (30.7 mg, 12.4% yield) as colorless solid. LC/MS: MS (ESI) calcd for C₂₆H₃₄F₃N₅O₂: 505.27, found: m/z=506.25 [M+H]⁺. ¹H NMR (400 MHz, Methanol-d₄): δ 7.42-7.40 (m, 1H), 7.24-7.22 (m, 1H), 7.18 (s, 1H), 6.78 (s, 1H), 4.14-4.10 (m, 1H), 3.94-3.90 (m, 1H), 3.22-3.21 (m, 1H), 3.18-2.97 (m, 2H), 2.85-2.79 (m, 1H), 2.53-2.41 (m, 4H), 2.36-2.22 (m, 2H), 2.15 (s, 3H), 2.10-1.95 (m, 1H), 1.91-1.57 (m, 10H), 1.49-1.38 (m, 1H) ppm. ¹⁹F-NMR (376 MHz, Methanol-d₄): δ −64.28 ppm.

A second fraction with RT2: 13.76 min, affording a single stereoisomer (randomly assigned “trans”) 2-(4-methyl-6-{[(3R)-1′-[(1r,3r)-3-hydroxycyclobutyl]-[1,4′-bipiperidin]-3-yl]amino}pyridazin-3-yl)-5-(trifluoromethyl)phenol (38b) (30.3 mg, 26.0% yield) as colorless solid. LC/MS: MS (ESI) calcd for C₂₆H₃₄F₃N₅O₂: 505.27; found: m/z=506.25 [M+H]⁺. ¹H NMR (400 MHz, Methanol-d₄) δ 7.40-7.37 (m, 1H), 7.22-7.16 (m, 2H), 6.76 (s, 1H), 4.15-4.05 (m, 1H), 3.93-3.88 (m, 1H), 3.25-3.18 (m, 1H), 3.06-2.96 (m, 2H), 2.88-2.78 (m, 1H), 2.47-2.32 (m, 4H), 2.29-2.23 (m, 2H), 2.12 (s, 3H), 2.03-1.99 (m, 1H), 1.97-1.86 (m, 4H), 1.86-1.76 (m, 3H), 1.76-1.73 (m, 2H), 1.58-1.40 (m, 2H) ppm. ¹⁹F-NMR (376 MHz, Methanol-d₄): δ −64.28 ppm.

### Example 39: 2-(4-Methyl-6-{[(3R)-1′-[(1r,4r)-4-hydroxycyclohexyl]-[1,4′-bipiperidin]-3-yl]amino}pyridazin-3-yl)-5-(trifluoromethyl)phenol (39a) and 2-(4-Methyl-6-{[(3R)-1′-[(1s,4S)-4-hydroxycyclohexyl]-[1,4′-bipiperidin]-3-yl]amino}pyridazin-3-yl)-5-(trifluoromethyl)phenol (39b)

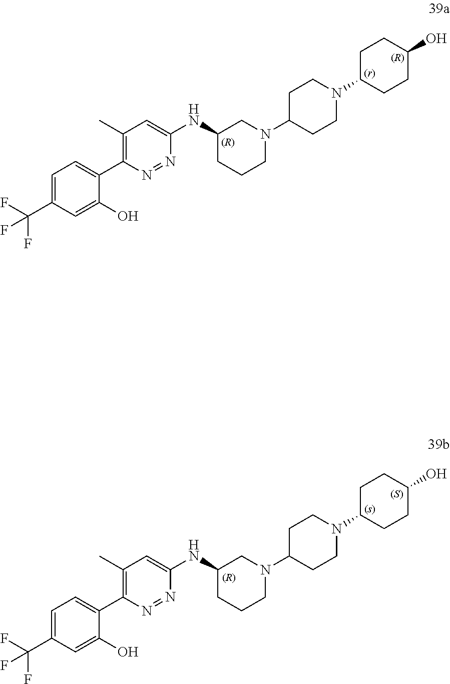

### Synthetic Route:

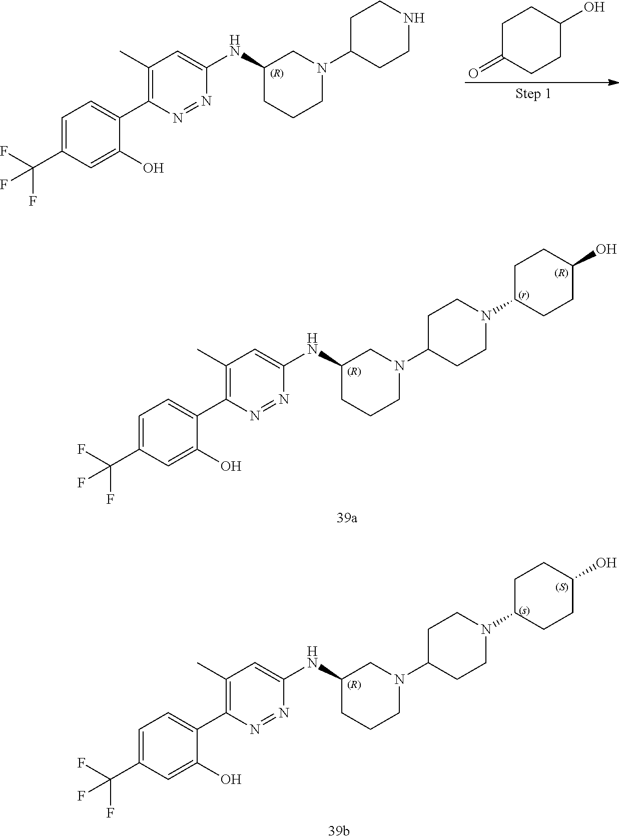

### Step 1: Synthesis of 2-(4-methyl-6-{[(3R)-1′-[(1r,4r)-4-hydroxycyclohexyl]-[1,4′-bipiperidin]-3-yl]amino}pyridazin-3-yl)-5-(trifluoromethyl)phenol (39a) and 2-(4-methyl-6-{[(3R)-1′-[(1s,4s)-4-hydroxycyclohexyl]-[1,4′-bipiperidin]-3-yl]amino}pyridazin-3-yl)-5-(trifluoromethyl)phenol (39b)

To a lightly yellow solution of 2-{6-[(3R)-[1,4′-bipiperidin]-3-ylamino]-4-methylpyridazin-3-yl}-5-(trifluoromethyl)phenol (300 mg, 0.69 mmol, 1.0 eq.) in MeOH (3 mL) were added 4-hydroxycyclohexan-1-one (90 mg, 0.76 mmol, 1.1 eq.) and NaBH₃CN (130 g, 2.07 mmol, 3.0 eq.). After stirring for 2 h at rt, the suspension was concentrated under reduced pressure using a rotary evaporator. The residue was purified by reverse flash chromatography using the following conditions: column, C18 silica gel; mobile phase, ACN in water, 10% to 60% gradient in 20 min; detector, UV 254 nm. Subsequent separation by SFC using the following condition: Column: CHIRALPAK IG, 2*25 cm, 5 μm; Mobile Phase A: Hex (0.5% 2 M NH₃-MeOH)—HPLC, Mobile Phase B: EtOH-HPLC; Flow rate: 20 mL/min; Gradient: 30% B to 30% B in 17 min; Wavelengths: 220/254 nm. Isolated at RT2: 13.90 min as a single stereoisomer (randomly assigned “trans”) 2-(4-methyl-6-{[(3R)-1′-[(1r,4r)-4-hydroxycyclohexyl]-[1,4′-bipiperidin]-3-yl]amino}pyridazin-3-yl)-5-(trifluoromethyl)phenol (39a) (39.9 mg, 10.7% yield) as colorless solid. LC/MS: MS (ESI) calcd for C₂₈H₃₈F₃N₅O₂: 533.30; found: m/z=534.40 [M+H]⁺. ¹H NMR (400 MHz, Methanol-d₄): δ=7.38-7.36 (m, 1H), 7.20-7.18 (m, 1H), 7.14 (s, 1H), 6.76 (s, 1H), 4.09 (s, 1H), 3.90 (s, 1H), 3.23-3.19 (m, 1H), 3.09-3.02 (m, 2H), 2.85-2.73 (m, 1H), 2.43-2.25 (m, 5H), 2.26-2.18 (m, 1H), 2.12 (s, 3H), 2.05-1.95 (m, 1H), 1.91-1.74 (m, 5H), 1.72-1.59 (m, 7H), 1.58-1.41 (m, 3H) ppm. ¹⁹F-NMR (376 MHz, Methanol-d₄): δ −64.22 ppm.

Isolated at RT1: 10.49 min as a single stereoisomer (randomly assigned “cis”) 2-(4-methyl-6-{[(3R)-1′-[(1s,4s)-4-hydroxycyclohexyl]-[1,4′-bipiperidin]-3-yl]amino}pyridazin-3-yl)-5-(trifluoromethyl)phenol (39b) (48.4 mg, 13.1% yield) as colorless solid. LC/MS: MS (ESI) calcd for C₂₈H₃₈F₃N₅O₂: 533.30; found: m/z=534.40 [M+H]⁺. ¹H NMR (400 MHz, Methanol-d₄): δ 7.41-7.30 (m, 1H), 7.23-7.16 (m, 1H), 7.14-7.09 (m, 1H), 6.75 (s, 1H), 4.11 (s, 1H), 3.53-3.41 (m, 1H), 3.25-3.13 (m, 1H), 3.10-3.01 (m, 2H), 2.88-2.75 (m, 1H), 2.51-2.29 (m, 5H), 2.27-2.18 (m, 1H), 2.12 (s, 3H), 2.03-1.85 (m, 7H), 1.81-1.76 (m, 1H), 1.75-1.57 (m, 3H), 1.18-1.11 (m, 5H) ppm. ¹⁹F-NMR (376 MHz, Methanol-d₄): δ −64.22 ppm.

### Example 40: (R)-(3-((6-(2-Hydroxy-4-(trifluoromethyl)phenyl)-5-methylpyridazin-3-yl)amino)-[1,4′-bipiperidin]-1′-yl) (3-hydroxyazetidin-1-yl)methanone (40)

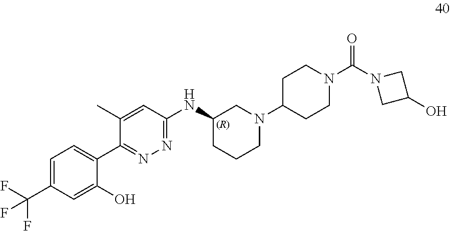

### Synthetic Scheme:

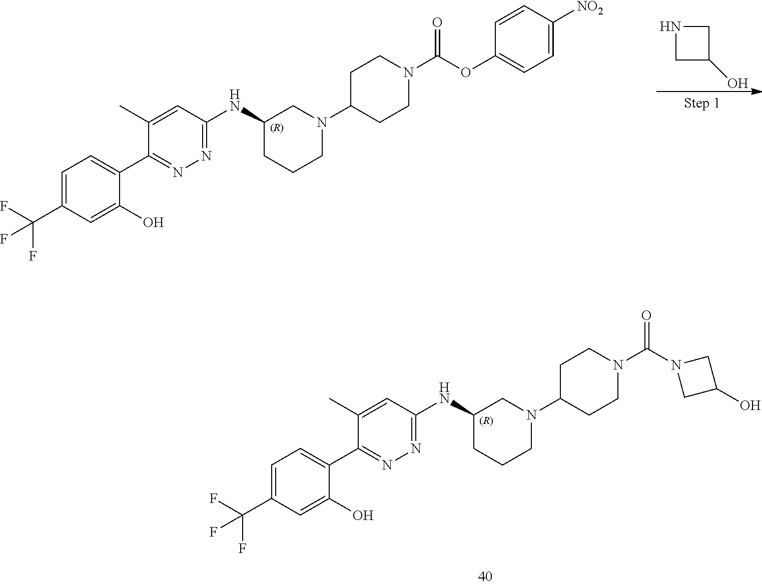

### Step 1: Synthesis of 1-[(3R)-3-({6-[2-hydroxy-4-(trifluoromethyl)phenyl]-5-methylpyridazin-3-yl}amino)-[1,4′-bipiperidine]-1′-carbonyl]azetidin-3-ol (40)

Into a 25 mL flask was added 4-nitrophenyl (3R)-3-({6-[2-hydroxy-4-(trifluoromethyl)phenyl]-5-methylpyridazin-3-yl}amino)-[1,4′-bipiperidine]-1′-carboxylate (250 mg, 0.42 mmol, 1.0 eq.), azetidin-3-ol (46 mg, 0.63 mmol, 1.5 eq.) and ACN (3 mL). The reaction mixture was stirred at rt for 10 min, then DIEA (340 μL, 252 mg, 1.95 mmol, 4.7 eq.) was added and the reaction mixture was stirred at 80° C. for 16 h in a sealed tube. Monitoring the reaction progress by LC/MS showed that about 70% of the product was generated. The reaction mixture was concentrated under reduced pressure using a rotary evaporator. The residue was purified by reversed-phase flash chromatography using the following conditions: column, C18 silica gel; mobile phase, ACN in Water (0.1 vol-% NH₃ aq.), 10% to 50% gradient in 10 min; detector, UV 254 nm to afford the target compound 1-[(3R)-3-({6-[2-hydroxy-4-(trifluoromethyl)phenyl]-5-methylpyridazin-3-yl}amino)-[1,4′-bipiperidine]-1′-carbonyl]azetidin-3-ol (40) (29.3 mg, 13.2%) as a colorless solid after lyophilization of the combined product containing fractions. LC/MS: mass calcd for C₂₆H₃₃F₃N₆O₃: 534.26, found: m/z=535.30 [M+H]⁺. ¹H NMR (400 MHz, DMSO-d₆): δ 10.45 (s, 1H), 7.41-7.39 (m, 1H), 7.24-7.20 (m, 2H), 6.69 (s, 1H), 6.59-6.57 (m, 1H), 5.52-5.50 (m, 1H), 4.40-4.30 (m, 1H), 4.04-4.00 (m, 3H), 3.77-3.63 (m, 2H), 3.63-3.60 (m, 2H), 3.02-2.96 (m, 1H), 2.62-2.48 (m, 3H), 2.48-2.42 (m, 1H), 2.30-2.20 (m, 1H), 2.15-2.10 (m, 1H), 2.02 (s, 3H), 1.90-1.80 (m, 1H), 1.69-1.66 (m, 3H), 1.60-1.40 (m, 1H), 1.38-1.28 (m, 3H) ppm. ¹⁹F NMR (376 MHz, DMSO-d₆): δ −61.17 ppm.

### Example 41: (R)-(3-((6-(2-Hydroxy-4-(trifluoromethyl)phenyl)-5-methylpyridazin-3-yl)amino)-[1,4′-bipiperidin]-1′-yl) (4-hydroxypiperidin-1-yl)methanone (41)

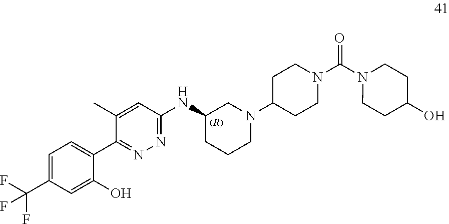

### Synthetic Route:

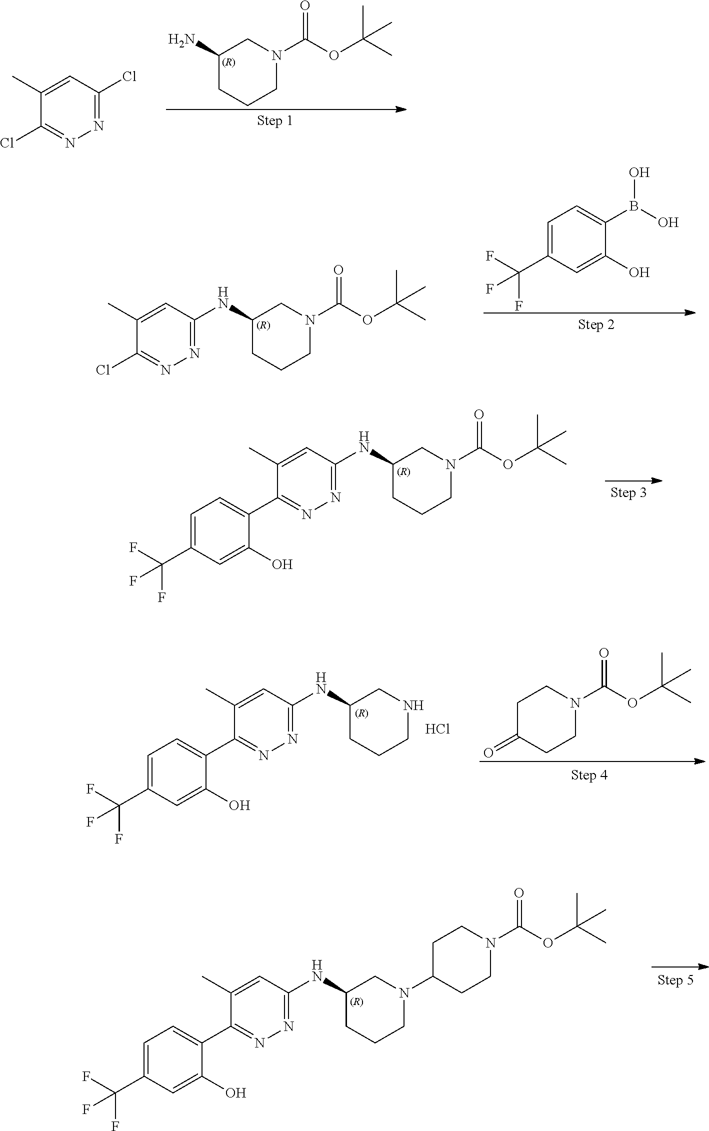

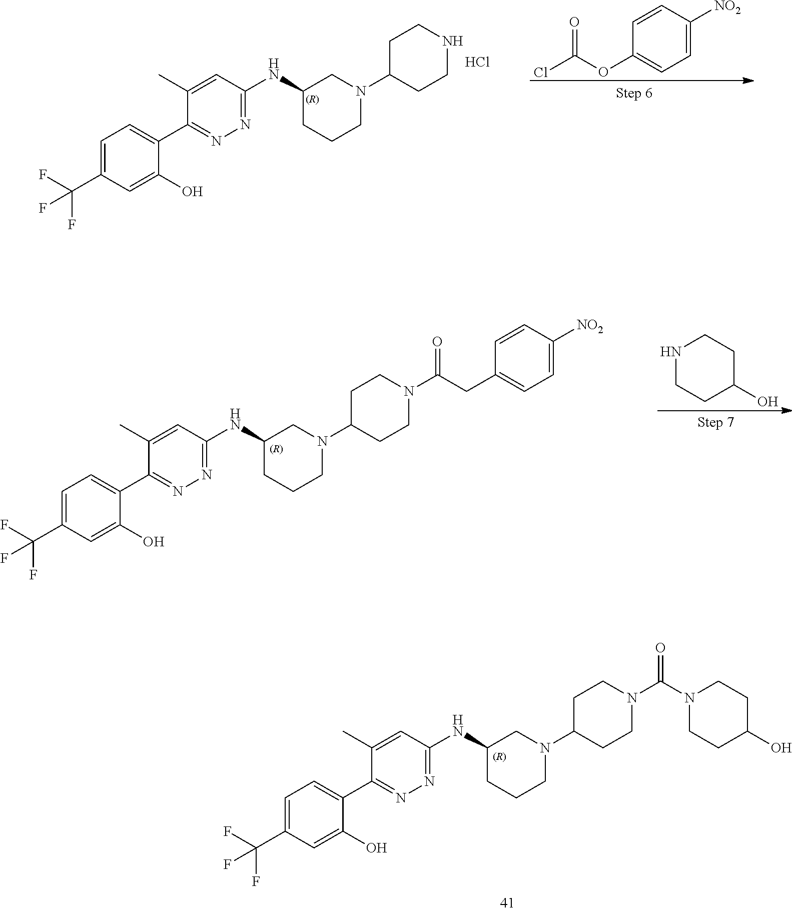

### Step 1: Synthesis of tert-Butyl (3R)-3-[(6-chloro-5-methylpyridazin-3-yl)amino]piperidine-1-carboxylate

A mixture of 3,6-dichloro-4-methylpyridazine (6.0 g, 36.8 mmol, 1.0 eq.) and tert-butyl (3R)-3-aminopiperidine-1-carboxylate (7.4 g, 36.8 mmol, 1.0 eq.) and DIEA (14 mL, 10.3 g, 80.4 mmol, ca. 2.2 eq.) in NMP (50 mL) was stirred at 150° C. for 18 h. The reaction progress was monitored by LC/MS. The reaction mixture was quenched with a water/ice slush (100 mL) and extracted with EA (3×50 mL). The combined organic layers were washed with water (3×50 mL) and brine (1×100 mL), and dried over anhydrous Na₂SO₄. After filtration, the filtrate was concentrated under reduced pressure using a rotary evaporator. The residue was purified by silica gel column chromatography eluting with a mixture PE and EA (1:1, v/v) to afford tert-butyl (3R)-3-[(6-chloro-5-methylpyridazin-3-yl)amino]piperidine-1-carboxylate (2.9 g, 24.1%) as a yellow oil after evaporation of the combined product containing fractions under reduced pressure using a rotary evaporator. LC/MS: mass calcd for C₁₅H₂₃ClN₄O₂: 326.15, found: 327.20 [M+H]⁺.

### Step 2: Synthesis of tert-Butyl (3R)-3-({6-[2-hydroxy-4-(trifluoromethyl)phenyl]-5-methylpyridazin-3-yl}amino)piperidine-1-carboxylate

To a solution of tert-butyl (3R)-3-[(6-chloro-5-methylpyridazin-3-yl)amino]piperidine-1-carboxylate (2.9 g, 8.9 mmol, 1.0 eq.) in 1,4-dioxane (30 mL) and H₂O (7.5 mL) was added 2-hydroxy-4 (trifluoromethyl)phenylboronic acid (2.2 g, 10.6 mmol, 1.2 eq.), Pd(PPh₃)₄ (1.03 g, 0.89 mmol, 0.1 eq.) and NaHCO₃ (1.49 g, 17.7 mmol, 2.0 eq.). The reaction mixture was stirred for 3 h at 100° C. After cooling to rt, the reaction mixture was filtered, and the filtrate was concentrated under reduced pressure using a rotary evaporator. The residue was purified by silica gel column chromatography, eluting with PE/EA mixture (12%) to afford tert-butyl (3R)-3-({6-[2-hydroxy-4-(trifluoromethyl)phenyl]-5-methylpyridazin-3-yl}amino)piperidine-1-carboxylate (2.3 g, 57.3%) as a yellow oil after evaporation of the combined product containing fractions under reduced pressure using a rotary evaporator. LC/MS: MS (ESI) calcd, for C₂₂H₂₇F₃N₄O₃: 452.20, found: 453.95 [M+H]⁺.

### Step 3: Synthesis of 2-{4-methyl-6-[(3R)-piperidin-3-ylamino]pyridazin-3-yl}-5-(trifluoromethyl)phenol

Into 250 mL flask was added tert-butyl (3R)-3-({6-[2-hydroxy-4-(trifluoromethyl)phenyl]-5-methylpyridazin-3-yl}amino)piperidine-1-carboxylate (2.3 g, 5.1 mmol), EA (30 mL), and HCl (10 mL, 40 mmol, 4 M in 1,4-dioxane). The reaction was stirred at room temperature for 1 h, and was monitored by LC/MS. After completion, the reaction mixture was concentrated under reduced pressure using a rotary evaporator to afford 2-{4-methyl-6-[(3R)-piperidin-3-ylamino]pyridazin-3-yl}-5-(trifluoromethyl)phenol (1.8 g, 94.9% yield) as a yellow solid. LC/MS: MS (ESI) calcd for C₁₇H₁₉F₃N₄O: 352.15, found: 353.10 [M+H]⁺.

### Step 4: Synthesis of tert-butyl (3R)-3-({6-[2-hydroxy-4-(trifluoromethyl)phenyl]-5-methylpyridazin-3-yl}amino)-[1,4′-bipiperidine]-1′-carboxylate

To a stirred solution of 2-{4-methyl-6-[(3R)-piperidin-3-ylamino]pyridazin-3-yl}-5-(trifluoromethyl)phenol (1.8 g, 5.1 mmol, 1.0 eq.) in MeOH (20 mL) was added tert-butyl 4-oxopiperidine-1-carboxylate (3.1 g, 15.3 mmol, 3.0 eq.) and NaBH₃CN (1.0 g, 15.3 mmol, 3.0 eq.) in portions at rt. The resulting reaction mixture was stirred for 16 h at 70° C. The reaction mixture was poured into water (30 mL) and extracted with EA (3×20 mL). The combined organic phases were dried over anhydrous Na₂SO₄, filtered, and the filtrate was concentrated under reduced pressure using a rotary evaporator. The residue was purified by silica gel column chromatography eluting with a PE/EA mixture (90%) to afford tert-butyl (3R)-3-({6-[2-hydroxy-4-(trifluoromethyl)phenyl]-5-methylpyridazin-3-yl}amino)-[1,4′-bipiperidine]-1′-carboxylate (1.2 g, 43.9%) as a yellow oil after evaporation of the combined product containing fractions under reduced pressure using a rotary evaporator. LC/MS: MS (ESI) calcd for C₂₇H₃₆F₃N₅O₃: 535.28, found: 536.35 [M+H]⁺.

### Step 5: Synthesis of 2-{6-[(3R)-[1,4′-bipiperidin]-3-ylamino]-4-methylpyridazin-3-yl}-5-(trifluoromethyl)phenol

Into a 50 ml flask was added tert-butyl (3R)-3-({6-[2-hydroxy-4-(trifluoromethyl)phenyl]-5-methylpyridazin-3-yl}amino)-[1,4′-bipiperidine]-1′-carboxylate (1.0 g, 1.9 mmol), EA (10 mL), and HCl (2 mL, 8 mmol, 4 M in 1,4-dioxane), the reaction was stirred at room temperature for 3 h and was monitored by LC/MS. Upon completion, the reaction mixture was concentrated under reduced pressure using a rotary evaporator to afford 2-{6-[(3R)-[1,4′-bipiperidin]-3-ylamino]-4-methylpyridazin-3-yl}-5-(trifluoromethyl)phenol (900 mg, ~110%) as a yellow solid. LC/MS: MS (ESI) calcd, for C₂₂H₂₈F₃N₅O: 435.22, found: 436.25 [M+H]⁺.

### Step 6: Synthesis of 4-nitrophenyl (R)-3-((6-(2-hydroxy-4-(trifluoromethyl)phenyl)-5-methylpyridazin-3-yl)amino)-[1,4′-bipiperidine]-1′-carboxylate

Into a 25 mL flask was added (R)-2-(6-([1,4′-bipiperidin]-3-ylamino)-4-methylpyridazin-3-yl)-5-(trifluoromethyl)phenol (400 mg, 0.92 mmol, 1.0 eq.), TEA (193 μL, 140 mg, 1.38 mmol, 1.5 eq.) and DCM (5 mL) at 0° C. To this solid 4-nitrophenyl carbonochloridate (4-nitrophenyl chloroformate) (221 mg, 1.01 mmol) was added and the reaction was stirred for 2 h at rt. The reaction mixture was quenched with a water/ice slurry (10 mL) and extracted with DCM (3×5 mL). The combined organic layers were dried over anhydrous Na₂SO₄, filtered, and the filtrate was concentrated under reduced pressure using a rotary evaporator. The residue was purified by silica gel column chromatography eluting with PE/EA gradients (0~30%) to afford 4-nitrophenyl (R)-3-((6-(2-hydroxy-4-(trifluoromethyl)phenyl)-5-methylpyridazin-3-yl)amino)-[1,4′-bipiperidine]-1′-carboxylate as a yellow solid after evaporation of the product containing fractions under reduced pressure using a rotary evaporator. LC/MS: mass calcd for C₂₉H₃₁F₃N₆O₅: 600.23, found: m/z=601.15 [M+H]⁺.

### Step 7: Synthesis of 1-[(3R)-3-({6-[2-hydroxy-4-(trifluoromethyl)phenyl]-5-methylpyridazin-3-yl}amino)-[1,4′-bipiperidine]-1′-carbonyl]piperidin-4-ol (41)

Into a 25 mL flask was added 4-nitrophenyl (3R)-3-({6-[2-hydroxy-4-(trifluoromethyl)phenyl]-5-methylpyridazin-3-yl}amino)-[1,4′-bipiperidine]-1′-carboxylate (250 mg, 0.42 mmol, 1.0 eq.), piperidin-4-ol (42 mg, 0.42 mmol, 1.0 eq.) and ACN (3 mL). The reaction mixture was stirred at rt for 10 min, then DIEA (300 μL, 223 mg, 1.72 mmol, 4.1 eq.) was added, the reaction was stirred at 80° C. for 16 h in a sealed tube. Monitoring the reaction progress by LC/MS showed that 70% of the product was formed. The volatiles were removed under reduced pressure and the residue was purified by reversed-phase flash chromatography with the following conditions: column, C18 silica gel; mobile phase, ACN in Water (0.1 vol-% NH₃ aq.), 10% to 50% gradient in 10 min; detector, UV 254 nm to afford the target compound 1-[(3R)-3-({6-[2-hydroxy-4-(trifluoromethyl)phenyl]-5-methylpyridazin-3-yl}amino)-[1,4′-bipiperidine]-1′-carbonyl]piperidin-4-ol (12.7 mg, 5.3%) as a yellow solid after lyophilization of the combined product containing fractions. LC/MS: mass calcd for C₂₈H₃₇F₃N₆O₃: 562.29, found: m/z=563.35 [M+H]⁺. ¹H NMR (400 MHz, DMSO-d₆): δ 10.45 (s, 1H), 7.41-7.39 (m, 1H), 7.24-7.20 (m, 2H), 6.69 (s, 1H), 6.61-6.59 (m, 1H), 4.66-4.65 (m, 1H), 4.04-4.00 (m, 1H), 3.62-3.50 (m, 3H), 3.42-3.35 (m, 2H), 3.10-3.00 (m, 1H), 2.84-2.79 (m, 2H), 2.69-2.63 (m, 3H), 2.30-2.20 (m, 1H), 2.15-2.10 (m, 1H), 2.02 (s, 3H), 1.90-1.80 (m, 1H), 1.75-1.60 (m, 5H), 1.60-1.40 (m, 1H), 1.38-1.28 (m, 5H). ¹⁹F NMR (376 MHz, DMSO-d₆): δ −61.17 ppm.

### Example 42: (R)-2-(3-(3-(2-Hydroxy-4-(trifluoromethyl)phenyl)-4-methyl-6,7-dihydropyrido[2,3-c]pyridazin-8 (5H)-yl)piperidin-1-yl)-1-(4-hydroxypiperidin-1-yl)ethan-1-one (42)

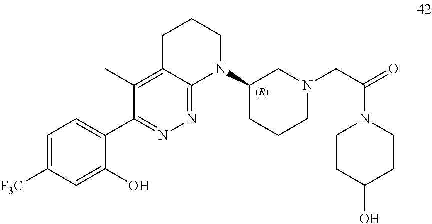

### Synthetic Route:

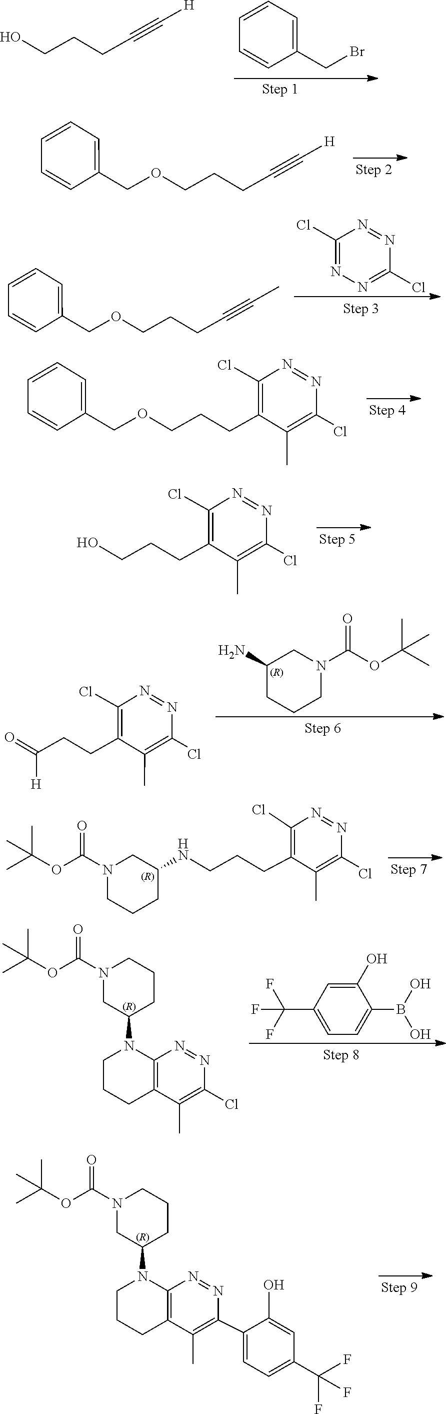

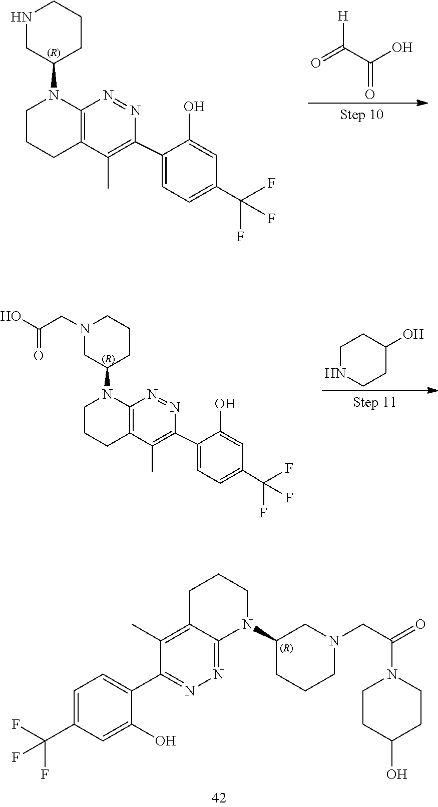

### Step 1: Synthesis of [(pent-4-yn-1-yloxy)methyl]benzene

A solution of pent-4-yn-1-ol (33.2 mL, 30.0 g, 357 mmol, 1.0 eq.) in THF (500 mL) was treated with NaH (15.7 g (9.42 g) of 60 wt-% NaH in mineral oil, 392 mmol, 1.1 eq.) for 30 min at 0° C. under an atmosphere of N₂ followed by dropwise addition of benzyl bromide (42.4 mL, 61.0 g, 357 mmol, 1.0 eq.) at 0° C. The resulting mixture was stirred for 16 h at rt under nitrogen atmosphere. Then, the mixture was allowed to cool down to 0° C. (ice bath). The reaction was quenched by the addition of water (1,000 mL) at 0° C. The resulting mixture was extracted with EA (3×500 mL). The combined organic layers were washed with brine (1×1,000 mL), dried over anhydrous Na₂SO₄. After filtration, the filtrate was concentrated under reduced pressure using a rotary evaporator. The residue was purified by silica gel column chromatography eluting with PE/EA gradients (0~5%) to afford [(pent-4-yn-1-yloxy)methyl]benzene (60.0 g, 96.6%) as a light brown oil. LC/MS: MS (ESI) calcd for Cl₂H₁₄O: 174.10. Found: m/z=175.00 [M+H]⁺.

### Step 2: Synthesis of [(hex-4-yn-1-yloxy)methyl]benzene

To an oven-dried flask was added [(pent-4-yn-1-yloxy)methyl]benzene (31.6 g, 181 mmol, 1.0 eq.) and THF (17 mL) and the solution was cooled to −78° C. (dry ice/acetone bath). n-butyllithium (n-BuLi) (88 mL, 934 mol, ca. 11 M in hexanes, ca. 5.2 eq.) was added dropwise and the reaction was stirred for 1 h then iodomethane (MeI) (300 mL, 684 g, 4.82 mol, ca. 27 eq.) was added dropwise, and the reaction mixture was allowed to warm to 0° C. over a period of about 1 h. The reaction was quenched by the addition of a saturated aqueous NH₄Cl solution (300 mL), extracted with EA (3×200 mL). The combined organic phase were dried over anhydrous Na₂SO₄, filtered, and the filtrate was evaporated under reduced pressure using a rotary evaporator to afford [(hex-4-yn-1-yloxy)methyl]benzene (30 g, 87.9%) as a brown oil. The crude product was used directly in the next step without further purification, isolation and characterization. LC/MS: mass calcd for C₁₃H₁₆O: 188.12. Found: m/z=189.15 [M+H]⁺.

### Step 3: Synthesis of 4-[3-(benzyloxy)propyl]-3,6-dichloro-5-methylpyridazine

To a stirred mixture of [(hex-4-yn-1-yloxy)methyl]benzene (5.00 g, 26.6 mmol, 1.0 eq.) in toluene (70 mL) was added 3,6-dichloro-1,2,4,5-tetrazine (4.00 g, 26.6 mmol, 1.0 eq.) in portions at rt. The resulting reaction mixture was stirred overnight at 110° C. The resulting reaction mixture was concentrated under reduced pressure using a rotary evaporator. The residue was purified by silica gel column chromatography eluting with PE/EA gradients (0~3%) to afford 4-[3-(benzyloxy) propyl]-3,6-dichloro-5-methylpyridazine (4.0 g, 48.4%) as a brown oil after evaporation of the combined product containing fractions under reduced pressure using a rotary evaporator. LC/MS: mass calcd for C₁₅H₁₆Cl₂N₂O: 310.16. Found: m/z=311.10 [M+H]⁺.

### Step 4: Synthesis of 3-(3,6-dichloro-5-methylpyridazin-4-yl) propan-1-ol

To a stirred mixture of 4-[3-(benzyloxy) propyl]-3,6-dichloro-5-methylpyridazine (13.8 g, 44.3 mmol, 1.0 eq.) in DCM (80 mL) was added BCl₃ (66 g, 563 mmol, 12.70 equiv) at 0° C. The resulting reaction mixture was stirred overnight at rt. The reaction was quenched by addition of a water/ice slush (30 mL). The quenched reaction mixture was neutralized to pH~7 with a saturated aqueous NaHCO₃ solution. The resulting mixture was extracted with DCM (3×40 mL). The combined organic layers were dried over anhydrous Na₂SO₄. After filtration, the filtrate was concentrated under reduced pressure using a rotary evaporator. The residue was purified by silica gel column chromatography eluting with PE/EA gradients (0~50%) to afford 3-(3,6-dichloro-5-methylpyridazin-4-yl) propan-1-ol (6.0 g, 61.2%) as a brown oil after evaporation of the combined product containing fractions. LC/MS: mass calcd for C₈H₁₀Cl₂N₂O: 220.02. Found: m/z=220.95 [M+H]⁺.

### Step 5: Synthesis of 3-(3,6-dichloro-5-methylpyridazin-4-yl)propanal

To an oven-dried flask was added DMSO (12.0 mL, 169 mmol, 15 eq.) and DCM (25 mL) and the solution was cooled to −78° C. Oxalylchloride (COCl)₂ (9.50 mL, 14.1 g, 111.5 mmol, 9.9 eq.) was added dropwise and the reaction was stirred for 1.5 h followed by dropwise addition of 3-(3,6-dichloro-5-methylpyridazin-4-yl) propan-1-ol (2.5 g, 11.3 mmol, 1.0 eq.) and TEA (23.6 mL, 17.1 g, 170 mmol, 15 eq.). The mixture was allowed to warm to room temperature. The reaction was quenched by the addition of water/ice (40 mL). The mixture was neutralized to pH~7 with a saturated aqueous NaHCO₃ solution. The resulting mixture was extracted with DCM (3×40 mL). The combined organic layers were dried over anhydrous Na₂SO₄, filtered, and the filtrate was concentrated under reduced pressure using a rotary evaporator to afford 3-(3,6-dichloro-5-methylpyridazin-4-yl)propanal (1.5 g, 60.6%) as a brown oil. The crude product was used directly in the next step without further purification, isolation, and characterization. LC/MS: MS (ESI) calcd for C₈H₈Cl₂N₂O: 218.00. Found: m/z=219.05 [M+H]⁺.

### Step 6: Synthesis of tert-butyl (3R)-3-{[3-(3,6-dichloro-5-methylpyridazin-4-yl) propyl]amino}piperidine-1-carboxylate

To a stirred mixture of 3-(3,6-dichloro-5-methylpyridazin-4-yl)propanal (3.0 g, 13.694 mmol, 1.0 eq.) and tert-butyl (3R)-3-aminopiperidine-1-carboxylate (5.5 g, 27.461 mmol, 2.0 eq.) in MeOH (40 mL) were added NaBH₃CN (1.7 g, 27.053 mmol, ca. 2 eq.) at 0° C. (ice bath). The resulting mixture was stirred at rt. The resulting reaction mixture was concentrated under reduced pressure using a rotary evaporator. The residue was purified by silica gel column chromatography eluting with PE/EA gradients (0~50%) to afford tert-butyl (3R)-3-{[3-(3,6-dichloro-5-methylpyridazin-4-yl) propyl]amino}piperidine-1-carboxylate (1.5 g, 27.2%) as a yellow oil after evaporation of the combined product containing fractions under reduced pressure using a rotary evaporator. LC/MS: MS (ESI) calcd for C₂₄H₃₀N₄O₃S: 402.16. Found: m/z=403.15 [M+H]⁺.

### Step 7: Synthesis of tert-butyl (3R)-3-{3-chloro-4-methyl-5H,6H,7H-pyrido[2,3-c]pyridazin-8-yl}piperidine-1-carboxylate

To a stirred mixture of tert-butyl (3R)-3-{[3-(3,6-dichloro-5-methylpyridazin-4-yl) propyl]amino}piperidine-1-carboxylate (760 mg, 1.884 mmol) in N-methyl pyrrolidinone (NMP) (7 mL) were added DIEA (623 μL, 462 mg 3.58 mmol) at rt. The resulting reaction mixture was stirred at rt. The resulting mixture was extracted with EA (30 mL×3). The combined organic layers were washed with H₂O (3×40 mL), and dried over anhydrous Na₂SO₄. After filtration, the filtrate was concentrated under reduced pressure using a rotary evaporator. The residue was purified by silica gel column chromatography eluting with PE/EA gradients (0~50%) to afford tert-butyl (3R)-3-{3-chloro-4-methyl-5H,6H,7H-pyrido[2,3-c]pyridazin-8-yl}piperidine-1-carboxylate (260 mg, 37.6%) as grey solid after evaporation of the combined product containing fractions under reduced pressure using a rotary evaporator. LC/MS: MS (ESI) calcd, for C₁₈H₂₇ClN₄O₂: 366.18. Found: m/z=367.20 [M+H]⁺.

### Step 8: Synthesis of tert-butyl (3R)-3-{3-[2-hydroxy-4-(trifluoromethyl)phenyl]-4-methyl-5H,6H,7H-pyrido[2,3-c]pyridazin-8-yl}piperidine-1-carboxylate

A solution of tert-butyl (3R)-3-{3-chloro-4-methyl-5H,6H,7H-pyrido[2,3-c]pyridazin-8-yl}piperidine-1-carboxylate (560 mg, 1.53 mmol, 1.0 eq.) in 1,4-dioxane (2 mL) was treated with 2-hydroxy-4-(trifluoromethyl)phenylboronic acid (371 mg, 1.80 mmol, ca. 1.2 eq.) for 5 min at rt temperature under an atmosphere of N₂ followed by addition of NaHCO₃ (258 mg, 3.07 mmol, 2.0 eq.) as a solution in H₂O (1 mL) at room temperature. The resulting mixture was stirred for 16 h at 100° C. under nitrogen atmosphere. The resulting mixture was concentrated under reduced pressure using a rotary evaporator. The residue was purified by silica gel column chromatography eluting with PE/EA gradients (0~50%) to afford tert-butyl (3R)-3-{3-[2-hydroxy-4-(trifluoromethyl)phenyl]-4-methyl-5H,6H,7H-pyrido[2,3-c]pyridazin-8-yl}piperidine-1-carboxylate (300 mg, 39.9%) as a brown solid after evaporation of the combined product containing fractions. LC/MS: MS (ESI) calcd, for C₂₅H₃₁F₃N₄O₃: 492.23. Found: m/z=493.35 [M+H]⁺.

### Step 9: Synthesis of 2-{4-methyl-8-[(3R)-piperidin-3-yl]-5H,6H, 7H-pyrido[2,3-c]pyridazin-3-yl}-5-(trifluoromethyl)phenol Hydrochloride

To a stirred mixture of tert-butyl (3R)-3-{3-[2-hydroxy-4-(trifluoromethyl)phenyl]-4-methyl-5H,6H,7H-pyrido[2,3-c]pyridazin-8-yl}piperidine-1-carboxylate (350 mg, 0.71 mmol) dissolved in DCM (3 mL) was added TFA (1.5 mL, 33 vol-%) at 0° C. The resulting reaction mixture was stirred for 2 h at rt. After evaporation of the volatiles under reduced pressure using a rotary evaporator, 2-{4-methyl-8-[(3R)-piperidin-3-yl]-5H,6H,7H-pyrido[2,3-c]pyridazin-3-yl}-5-(trifluoromethyl)phenol hydrochloride (278 mg, quant.) was obtained as a brown solid. The crude product was used directly in the next step without further purification, isolation, and characterization. LC/MS: MS (ESI) calcd for C₂₀H₂₃F₃N₄O: 392.18. Found: m/z=393.25 [M+H]⁺.

### Step 10: Synthesis of [(3R)-3-{3-[2-hydroxy-4-(trifluoromethyl)phenyl]-4-methyl-5H,6H,7H-pyrido[2,3-c]pyridazin-8-yl}piperidin-1-yl]acetic Acid

To a stirred mixture of 2-{4-methyl-8-[(3R)-piperidin-3-yl]-5H,6H,7H-pyrido[2,3-c]pyridazin-3-yl}-5-(trifluoromethyl)phenol hydrochloride (520 mg, 1.33 mmol, 1.0 eq.) and glyoxylate (364 mg, 4.92 mmol) in MeOH (5 mL) was added NaBH₃CN (242 mg, 3.86 mmol, 2.9 eq.) at 0° C. The resulting reaction mixture was stirred overnight at rt, and the suspension was concentrated under reduced pressure using a rotary evaporator. The residue was purified by reversed-phase flash chromatography with the following conditions: column, C18 silica gel; mobile phase, ACN (10 mmol/L NH₄HCO₃), 10% to 50% gradient in 10 min; detector, UV 254 nm to afford [(3R)-3-{3-[2-hydroxy-4-(trifluoromethyl)phenyl]-4-methyl-5H,6H, 7H-pyrido[2,3-c]pyridazin-8-yl}piperidin-1-yl]acetic acid (50 mg, 8.4%) as a yellow oil after lyophilization of the combined product containing fractions. LC/MS: MS (ESI) calcd. for C₂₂H₂₅F₃N₄O₃: 450.19. Found: m/z=451.20 [M+H]⁺.

### Step 11: Synthesis of (R)-2-(3-(3-(2-hydroxy-4-(trifluoromethyl)phenyl)-4-methyl-6,7-dihydropyrido[2,3-c]pyridazin-8 (5H)-yl)piperidin-1-yl)-1-(4-hydroxypiperidin-1-yl)ethan-1-one (42)

A solution of [(3R)-3-{3-[2-hydroxy-4-(trifluoromethyl)phenyl]-4-methyl-5H,6H,7H-pyrido[2,3-c]pyridazin-8-yl}piperidin-1-yl]acetic acid (50 mg, 0.11 mmol, 1.0 eq.) and piperidin-4-ol (16.8 mg, 0.17 mmol) in DMF (3 mL) was treated with DIEA (37 μL, 27.5 mg, 0.21 mmol, 2 eq.) for 5 min at 0° C. under an atmosphere of N₂ followed by the addition of HATU (63 mg, 0.17 mmol, 1.5 eq.) in small portions at 0° C. The reaction mixture was stirred for 3 h at rt. The reaction mixture was diluted with 0.1 M HCl (10 mL), and the aqueous phase was extracted with EA (3×20 mL). The combined organic phases were dried over anhydrous Na₂SO₄, filtered, and the filtrate was evaporated under reduced pressure using a rotary evaporator. The crude product was purified by preparative RP-HPLC using the following conditions (Column: YMC-Actus Triart C18 ExRS30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 35% B to 65% B in 7 min; Wavelength: 254 nm; RT1: 5.68 min to afford 2-[(3R)-3-{3-[2-hydroxy-4-(trifluoromethyl)phenyl]-4-methyl-5H,6H,7H-pyrido[2,3-c]pyridazin-8-yl}piperidin-1-yl]-1-(4-hydroxypiperidin-1-yl) ethenone (42) (8.0 mg, 13.3%) as a white solid after lyophilization of the product containing fractions. LC/MS: MS (ESI) calcd for C₂₇H₃₄F₃N₅O₃: 533.26. Found: m/z=534.15 [M+H]⁺. ¹H NMR (400 MHz, DMSO-d₆): δ 7.45-7.32 (m, 1H), 7.29-7.20 (m, 1H), 7.15 (s, 1H), 4.50-4.25 (m, 1H), 4.05-3.80 (m, 1H), 3.70-3.52 (m, 4H), 3.20-3.05 (m, 1H), 3.02-2.65 (m, 6H), 2.42-2.20 (m, 1H), 2.10-2.00 (m, 6H), 1.95-1.70 (m, 8H), 1.56-1.32 (m, 1H). ¹⁹F NMR (376 MHz, DMSO-d₆): δ −64.23 ppm.

### Example 43: 2-[(3R)-3-{3-[2-Hydroxy-4-(trifluoromethyl)phenyl]-4-methyl-5H,6H,7H-pyrido[2,3-c]pyridazin-8-yl}piperidin-1-yl]-1-{2-hydroxy-7-azaspiro[3.5]nonan-7-yl}ethanone (43)

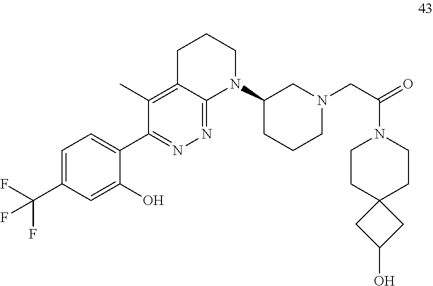 [SMILES: Cc1c(-c2ccc(C(F)(F)F)cc2O)nnc2c1CCCN2[C@@H]1CCCN(CC(=O)N2CCC3(CC2)CC(O)C3)C1]

### Synthetic Route:

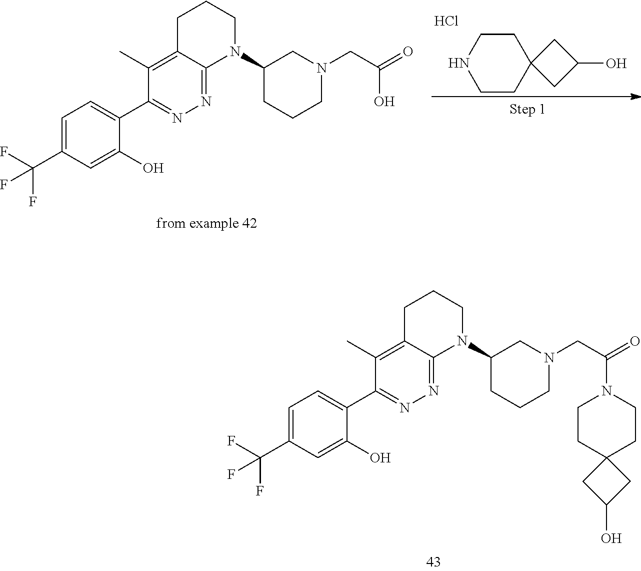 [SMILES: Cc1c(-c2ccc(C(F)(F)F)cc2O)nnc2c1CCCN2[C@@H]1CCCN(CC(=O)O)C1 ; Cl ; OC1CC2(CCNCC2)C1 ; Cc1c(-c2ccc(C(F)(F)F)cc2O)nnc2c1CCCN2[C@@H]1CCCN(CC(=O)N2CCC3(CC2)CC(O)C3)C1]

### Step 1: Synthesis of 2-[(3R)-3-{3-[2-hydroxy-4-(trifluoromethyl)phenyl]-4-methyl-5H,6H, 7H-pyrido[2,3-c]pyridazin-8-yl}piperidin-1-yl]-1-{2-hydroxy-7-azaspiro[3.5]nonan-7-yl}ethanone (43)

To a stirred mixture of [(3R)-3-{3-[2-hydroxy-4-(trifluoromethyl)phenyl]-4-methyl-5H,6H,7H-pyrido[2,3-c]pyridazin-8-yl}piperidin-1-yl]acetic acid (prepared as disclosed for Example 42) (40 mg, 0.1 mmol, 1.0 eq.) and 7-azaspiro[3.5]nonan-2-ol (25 mg, 0.2 mmol, 2.0 eq.) in pyridine (3 mL) was added EDCI (36 mg, 0.2 mmol, ~2.1 eq.) at rt. The resulting reaction mixture was stirred for 5 h at 50° C. (oil bath) and the resulting mixture was concentrated under reduced pressure using a rotary evaporator. The crude reaction product (80 mg) was purified by preparative RP-HPLC using the following conditions Column: XBridge Prep Phenyl OBD Column, 30*150 mm, 5 μm; Mobile Phase A: Water (10 mmol/L NH₄HCO₃+0.1 vol-% NH₃ aq.), Mobile Phase B: ACN; flow rate: 60 mL/min; Gradient: 31% B to 61% B in 7 min; wavelength: 254 nm; RT1: 5.69 min to afford the target compound 2-[(3R)-3-{3-[2-hydroxy-4-(trifluoromethyl)phenyl]-4-methyl-5H,6H,7H-pyrido[2,3-c]pyridazin-8-yl}piperidin-1-yl]-1-{2-hydroxy-7-azaspiro[3.5]nonan-7-yl}ethanone (43) (19 mg, 36.0%) as a light yellow solid after lyophilization of the combined product containing fractions. LC/MS: MS (ESI) calcd for C₃₀H₃₈F₃N₅O₃: 573.29. Found: m/z=574.30 [M+H]⁺. ¹H NMR (400 MHz, DMSO-d₆): δ 7.38 (d, J=8.0 Hz, 1H), 7.24 (d, J=8.0 Hz, 1H), 7.17 (s, 1H), 5.20-5.10 (m, 1H), 4.30-4.15 (m, 1H), 3.70-3.35 (m, 6H), 3.70-3.25 (m, 6H), 3.30-3.25 (m, 2H), 3.00-2.82 (m, 2H), 2.80-2.75 (m, 2H), 2.40-2.20 (m, 3H), 2.10-2.02 (m, 4H), 2.00-1.90 (m, 2H), 1.80-1.60 (m, 8H), 1.59-1.50 (m, 2H) ppm. ¹⁹F NMR (376 MHz, DMSO-d₆): δ −64.23 ppm.

### Example 44:2-{8-[(3R)-1′-(3-Hydroxycyclobutyl)-[1,4′-bipiperidin]-3-yl]-4-methyl-5H,6H,7H-pyrido[2,3-c]pyridazin-3-yl}-5-(trifluoromethyl)phenol (44)

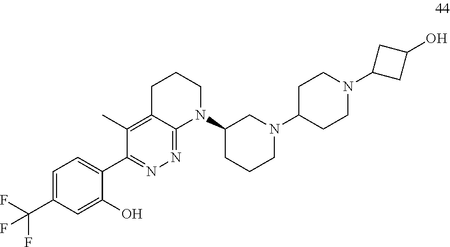 [SMILES: Cc1c(-c2ccc(C(F)(F)F)cc2O)nnc2c1CCCN2[C@@H]1CCCN(C2CCN(C3CC(O)C3)CC2)C1]

### Synthetic Route:

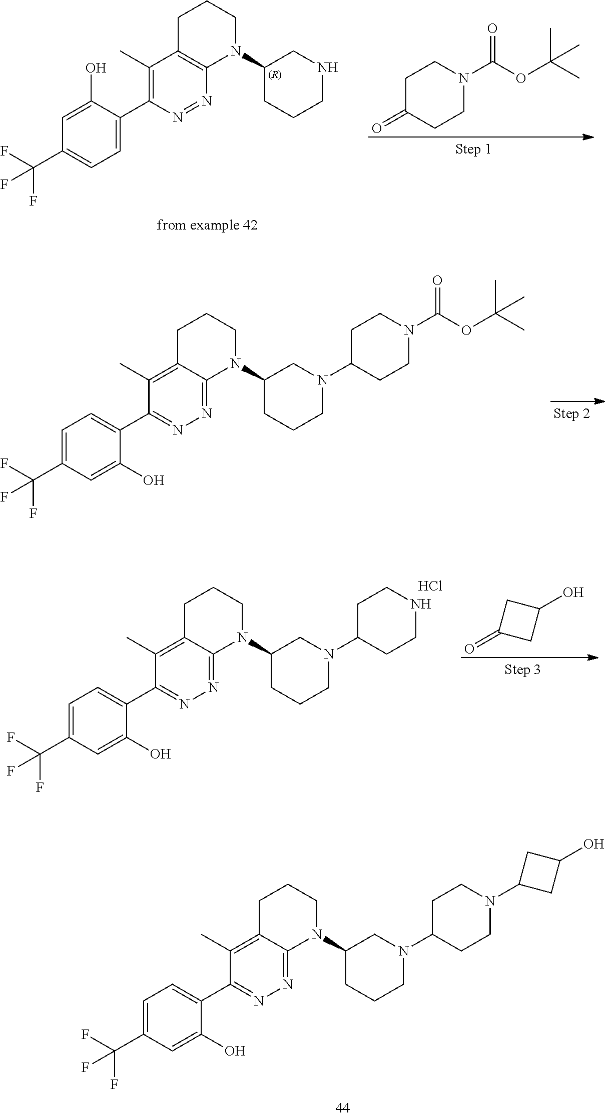

### Step 1: Synthesis of tert-butyl (3R)-3-{3-[2-hydroxy-4-(trifluoromethyl)phenyl]-4-methyl-5H,6H, 7H-pyrido[2,3-c]pyridazin-8-yl}-[1,4′-bipiperidine]-1′-carboxylate

To a stirred mixture of 2-{4-methyl-8-[(3R)-piperidin-3-yl]-5H,6H,7H-pyrido[2,3-c]pyridazin-3-yl}-5-(trifluoromethyl)phenol trifluoroacetate (1.2 g, 3.1 mmol, 1.0 eq.) (prepared as disclosed for Example 42) and tert-butyl 4-oxopiperidine-1-carboxylate (1.8 g, 9.0 mmol, 3.0 eq.) in MeOH (10 mL) was added solid NaBH₃CN (580 mg, 9.2 mmol, 3.0 eq.) at 0° C. (ice bath) in small portions. The resulting reaction mixture was stirred for 72 h at 45° C. (oil bath) followed by concentration under reduced pressure using a rotary evaporator. The residue was purified by silica gel column chromatography eluting using MeOH/DCM gradients (0~10%) to afford tert-butyl (3R)-3-{3-[2-hydroxy-4-(trifluoromethyl)phenyl]-4-methyl-5H,6H,7H-pyrido[2,3-c]pyridazin-8-yl}-[1,4′-bipiperidine]-1′-carboxylate (800 mg, 45.4%) as a yellow solid after evaporation of the combined product containing fractions under reduced pressure using a rotary evaporator. LC/MS: MS (ESI) calcd for C₃₀H₄₀F₃N₅O₃: 575.31. Found: m/z=576.35 [M+H]⁺.

### Step 2: Synthesis of 2-{8-[(3R)-[1,4′-bipiperidin]-3-yl]-4-methyl-5H,6H, 7H-pyrido[2,3-c]pyridazin-3-yl}-5-(trifluoromethyl)phenol hydrochloride

To a stirred mixture of tert-butyl (3R)-3-{3-[2-hydroxy-4-(trifluoromethyl)phenyl]-4-methyl-5H,6H,7H-pyrido[2,3-c]pyridazin-8-yl}-[1,4′-bipiperidine]-1′-carboxylate (800 mg, 1.4 mmol, 1.0 eq.) in THF (8 mL) was added HCl (4 M in 1,4-dioxane, 4 mL, 16 mmol, ~11 eq.) at 0° C. (ice bath). The resulting reaction mixture was stirred for 8 h at rt. After evaporation of the volatiles under reduced pressure using a rotary evaporator, 2-{8-[(3R)-[1,4′-bipiperidin]-3-yl]-4-methyl-5H,6H,7H-pyrido[2,3-c]pyridazin-3-yl}-5-(trifluoromethyl)phenol hydrochloride (880 mg, crude) was obtained as a brown solid. The crude product was used directly in the next step without further purification, isolation and characterization. LC/MS: MS (ESI) calcd for C₂₅H₃₂F₃N₅O: 475.26. Found: m/z=476.20 [M+H]⁺.

### Step 3: Synthesis of 2-{8-[(3R)-1′-(3-hydroxycyclobutyl)-[1,4′-bipiperidin]-3-yl]-4-methyl-5H,6H,7H-pyrido[2,3-c]pyridazin-3-yl}-5-(trifluoromethyl)phenol (44)

To a stirred mixture of 2-{8-[(3R)-[1,4′-bipiperidin]-3-yl]-4-methyl-5H,6H,7H-pyrido[2,3-c]pyridazin-3-yl}-5-(trifluoromethyl)phenol hydrochloride (50 mg, 0.10 mmol, 1.0 eq) and 3-hydroxycyclobutan-1-one (26 mg, 0.30 mmol, 3.1 eq.) in MeOH (3 mL) was added solid NaBH₃CN (18 mg, 0.29 mmol, ~2.9 eq.) at 0° C. (ice bath) in small portions. The resulting mixture was stirred for 36 h at 45° C. (oil bath) followed by concentration under reduced pressure using a rotary evaporator. The crude product (50 mg) was purified by preparative RP-HPLC using the following conditions Column XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 31% B to 43% B in 10 min; Wavelengths: 254 nm/220 nm; RT1: 9.68 min to afford the target compound 2-{8-[(3R)-1′-(3-hydroxycyclobutyl)-[1,4′-bipiperidin]-3-yl]-4-methyl-5H,6H,7H-pyrido[2,3-c]pyridazin-3-yl}-5-(trifluoromethyl)phenol (44) (5 mg, 9.1%) as a white solid after lyophilization of the combined solvent containing fractions. LC/MS: MS (ESI) calcd for C₂₉H₃₈F₃N₅O₂: 545.30. Found: m/z=546.35 [M+H]⁺. ¹H NMR (300 MHz, DMSO-d₆): δ 7.36 (d, J=9.9 Hz, 1H), 7.19 (d, J=7.5 Hz, 1H), 7.13 (s, 1H), 5.20-5.00 (m, 1H), 4.30-3.90 (m, 1H), 3.50-3.35 (m, 2H), 3.10-3.02 (m, 1H), 3.00-2.90 (m, 3H), 2.80-2.70 (m, 2H), 2.60-2.40 (m, 4H), 2.30-2.15 (m, 2H), 2.05 (s, 3H), 2.00-1.70 (m, 12H), 1.65-1.45 (m, 2H) ppm. ¹⁹F NMR (282 MHz, DMSO-d₆): δ −64.24 ppm.

### Example 45: 2-{4-Methyl-8-[(3R)-1′-[(1s,3s)-3-hydroxycyclobutyl]-[1,4′-bipiperidin]-3-yl]-5H,6H,7H-pyrido[2,3-c]pyridazin-3-yl}-5-(trifluoromethyl)phenol (45)

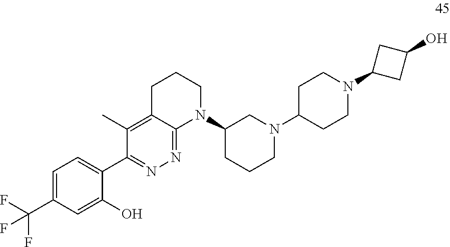 [SMILES: Cc1c(-c2ccc(C(F)(F)F)cc2O)nnc2c1CCCN2[C@@H]1CCCN(C2CCN([C@H]3C[C@@H](O)C3)CC2)C1]

### Synthetic Route:

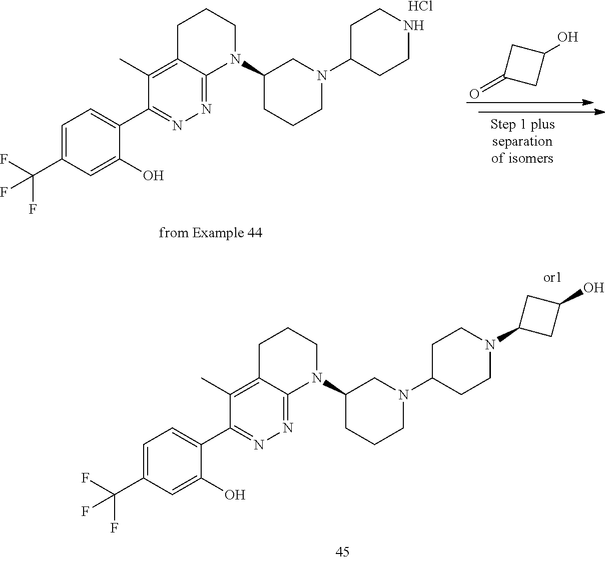 [SMILES: Cl ; Cc1c(-c2ccc(C(F)(F)F)cc2O)nnc2c1CCCN2[C@@H]1CCCN(C2CCNCC2)C1 ; O=C1CC(O)C1 ; Cc1c(-c2ccc(C(F)(F)F)cc2O)nnc2c1CCCN2[C@@H]1CCCN(C2CCN([C@H]3C[C@@H](O)C3)CC2)C1]

### Step 1: Synthesis of 2-{4-methyl-8-[(3R)-1′-[(1s,3s)-3-hydroxycyclobutyl]-[1,4′-bipiperidin]-3-yl]-5H,6H,7H-pyrido[2,3-c]pyridazin-3-yl}-5-(trifluoromethyl)phenol (45)

To a stirred mixture of 2-{8-[(3R)-[1,4′-bipiperidin]-3-yl]-4-methyl-5H,6H,7H-pyrido[2,3-c]pyridazin-3-yl}-5-(trifluoromethyl)phenol hydrochloride (prepared as disclosed for Example 44) (300 mg, 0.6 mmol, 1.0 eq.) and 3-hydroxycyclobutan-1-one (152 mg, 1.8 mmol, 3.0 eq.) in MeOH (5 mL) was added solid NaBH₃CN (110 mg, 1.8 mmol, 3.0 eq.) at 0° C. (ice bath) in small portions. The reaction mixture was stirred for 36 h at 45° C. followed by concentration under reduced pressure using a rotary evaporator. The crude product was purified by preparative ACHIRAL-SFC using the following conditions Column: DAICEL DCpak P4VP 3*25 cm, 5 μm; Mobile Phase A: CO₂, Mobile Phase B: ACN:MeOH=4:1 (v/v) (0.1 vol-% 2 M NH₃ in MeOH); Flow rate: 60 mL/min; Gradient: isocratic 42% B; Column Temperature: 35° C.; Back Pressure: 100 bar; Wavelength: 220 nm; RT1: 7.29 min; RT2: 9.21 min; Sample Solvent: MeOH—HPLC; Injection Volume: 2 mL; Number of runs: 16 to afford the target compound 2-{4-methyl-8-[(3R)-1′-[(1s,3s)-3-hydroxycyclobutyl]-[1,4′-bipiperidin]-3-yl]-5H,6H,7H-pyrido[2,3-c]pyridazin-3-yl}-5-(trifluoromethyl)phenol (45) (80 mg, 24.5%) as a white solid after lyophilization of the combined product containing fractions. LC/MS: MS (ESI) calcd for C₂₉H₃₈F₃N₅O₂: 545.30. Found: m/z=546.20 [M+H]⁺. ¹H NMR (300 MHz, DMSO-d₆): δ 7.37 (d, J=7.6 Hz, 1H), 7.20 (d, J=8.4 Hz, 1H), 7.15 (s, 1H), 5.18-5.02 (m, 1H), 3.99-3.80 (m, 1H), 3.48-3.38 (m, 2H), 3.18-2.95 (m, 4H), 2.82-2.65 (m, 2H), 2.60-2.42 (m, 4H), 2.40-2.20 (m, 2H), 2.02-1.50 (m, 17H) ppm. ¹⁹F NMR (282 MHz, DMSO-d₆): δ −64.27 ppm.

### Example 46: 3-[(3R)-3-({6-[2-Hydroxy-4-(trifluoromethyl)phenyl]-5-methylpyridazin-3-yl}amino)piperidin-1-yl]-1-(4-hydroxypiperidin-1-yl) propan-1-one (46)

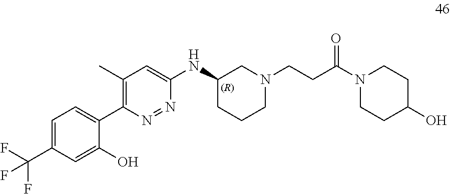

### Synthetic Route:

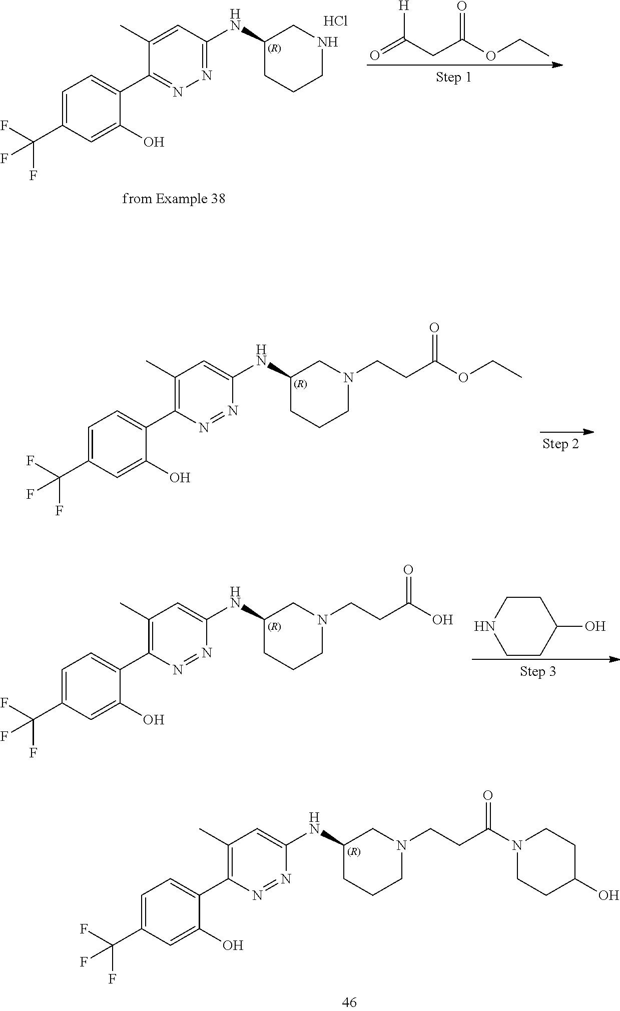

### Step 1: Synthesis of ethyl 3-[(3R)-3-({6-[2-hydroxy-4-(trifluoromethyl)phenyl]-5-methylpyridazin-3-yl}amino)piperidin-1-yl]propanoate

To a stirred solution of 2-{4-methyl-6-[(3R)-piperidin-3-ylamino]pyridazin-3-yl}-5-(trifluoromethyl)phenol (prepared as disclosed in Example 38) (400 mg, 1.1 mmol, 1.0 eq.) in MeOH (8 mL) were added ethyl 3-oxopropanoate (396 mg, 3.4 mmol, 3.0 eq.) and solid NaBH₃CN (214 mg, 3.4 mmol, 3.0 eq.) at 0° C. (ice bath). The resulting reaction mixture was stirred for 16 h at rt. The reaction mixture was quenched through addition of an ice/water slurry (5 mL) and was concentrated under reduced pressure using a rotary evaporator. The residue was directly purified by silica gel column chromatography eluting with MeOH/DCM (0~3%) gradients to afford ethyl 3-[(3R)-3-({6-[2-hydroxy-4-(trifluoromethyl)phenyl]-5-methylpyridazin-3-yl}amino)piperidin-1-yl]propanoate (370 mg, 72.0%) as a yellow solid after evaporation of the combined product containing fractions under reduced pressure using a rotary evaporator. LC/MS: MS (ESI) calcd for C₂₂H₂₇F₃N₄O₃: 452.20. Found: m/z=453.30 [M+H]⁺.

### Step 2: Synthesis of 3-[(3R)-3-({6-[2-hydroxy-4-(trifluoromethyl)phenyl]-5-methylpyridazin-3-yl}amino)piperidin-1-yl]propanoic Acid

A solution of ethyl 3-[(3R)-3-({6-[2-hydroxy-4-(trifluoromethyl)phenyl]-5-methylpyridazin-3-yl}amino)piperidin-1-yl]propanoate (350 mg, 0.8 mmol, 1.0 eq.) and LiOH·H₂O (65 mg, 1.5 mmol, 2.0 eq.) in a mixture of MeOH (2 mL), H₂O (2 mL), and THF (2 mL) was stirred for 5 min at 0° C. (ice bath) followed by stirring with gradual warming to rt for 3 h. The reaction progress was monitored by LC/MS analysis showing about 70% of product formation after that time. The reaction mixture was concentrated under reduced pressure using a rotary evaporator and diluted with water (2 mL). The pH value of the mixture was adjusted to about 5 with diluted hydrochloric acid (pH strips). The precipitated solids were collected by filtration and dried under reduced pressure to afford 3-[(3R)-3-({6-[2-hydroxy-4-(trifluoromethyl)phenyl]-5-methylpyridazin-3-yl}amino)piperidin-1-yl]propanoic acid (300 mg, crude) as a yellow solid which was used directly in the next step without further isolation, purification and characterization. LC/MS: mass calcd for C₂₀H₂₃F₃N₄O₃: 424.17. Found: m/z=425.20 [M+H]⁺.

### Step 3: Synthesis of 3-[(3R)-3-({6-[2-hydroxy-4-(trifluoromethyl)phenyl]-5-methylpyridazin-3-yl}amino)piperidin-1-yl]-1-(4-hydroxypiperidin-1-yl) propan-1-one (46)

To a stirred solution of 3-[(3R)-3-({6-[2-hydroxy-4-(trifluoromethyl)phenyl]-5-methylpyridazin-3-yl}amino)piperidin-1-yl]propanoic acid (350 mg, 0.8 mmol, 1.0 eq.) in pyridine (10 mL) were added piperidin-4-ol (100 mg, 1.0 mmol, 1.2 eq.) and EDCI (237 mg, 1.2 mmol, 1.5 eq.) at rt. The resulting reaction mixture was stirred for 16 h at 50° C. (oil bath). The reaction progress was monitored by LC/MS analysis showing that the reaction was completed after that time. The reaction mixture was concentrated under reduced pressure using a rotary evaporator. The residue was directly purified by preparative RP-HPLC using the following conditions XBridge Prep Phenyl OBD C18 Column, 30*150 mm, 5 μm; Mobile Phase A: Water (10 mmol/L NH₄HCO₃), Mobile Phase B: ACN; Flow rate: 60 mL/min; Gradient: 16% B to 41% B in 10 min; Wavelengths 254 nm/220 nm; RT1: 8.93 min to afford the target compound 3-[(3R)-3-({6-[2-hydroxy-4-(trifluoromethyl)phenyl]-5-methylpyridazin-3-yl}amino)piperidin-1-yl]-1-(4-hydroxypiperidin-1-yl) propan-1-one (46) (8.0 mg, 13.3%) as a colorless solid after lyophilization of the combined product containing fractions. LC/MS: MS (ESI) calcd for C₂₅H₃₂F₃N₅O₃: 507.24. Found: m/z=508.15 [M+H]⁺. ¹H NMR (400 MHz, Methanol-d₄): δ 7.41 (d, J=8.0 Hz, 1H), 7.23 (d, J=8.0 Hz, 1H), 7.17 (s, 1H), 6.78 (s, 1H), 4.20-4.00 (m, 2H), 3.90-3.80 (m, 2H), 3.35-3.30 (m, 1H), 3.20-3.10 (m, 2H), 3.70-3.50 (m, 5H), 2.35-2.10 (m, 5H), 2.02-1.80 (m, 4H), 1.70-1.60 (m, 1H), 1.55-1.45 (m, 3H) ppm. ¹⁹F NMR (376 MHz, Methanol-d₄): δ −64.27 ppm.

### Example 47: 3-[(3R)-3-({6-[2-Hydroxy-4-(trifluoromethyl)phenyl]-5-methylpyridazin-3-yl}amino)piperidin-1-yl]-1-(4-hydroxypiperidin-1-yl)-2,2-dimethylpropan-1-one (47)

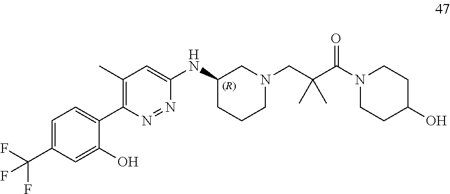

### Synthetic Route:

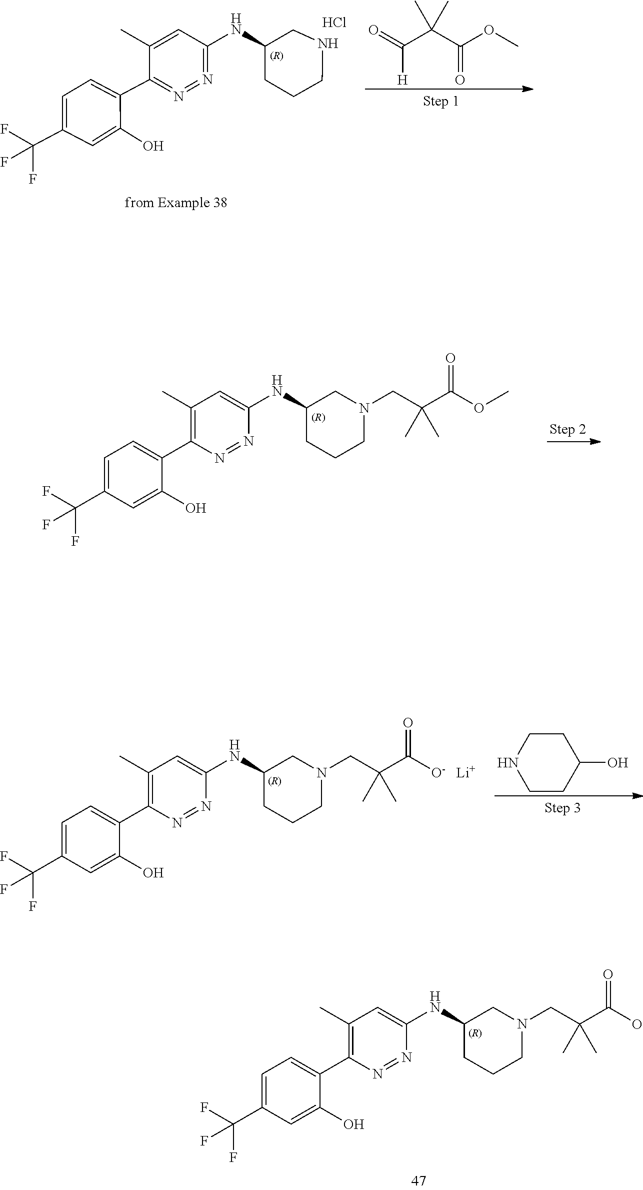

### Step 1: Synthesis of methyl 3-[(3R)-3-({6-[2-hydroxy-4-(trifluoromethyl)phenyl]-5-methylpyridazin-3-yl}amino)piperidin-1-yl]-2,2-dimethylpropanoate

A solution of 2-{4-methyl-6-[(3R)-piperidin-3-ylamino]pyridazin-3-yl}-5-(trifluoromethyl)phenol hydrochloride (prepared as disclosed in Example 38) (300 mg, 0.9 mmol, 1.0 eq.) in MeOH (5 mL) was acidified with AcOH (~1 drop) and stirred for 5 min at 0° C. (ice bath) under an atmosphere of followed by addition of solid NaBH₃CN (107 mg, 1.7 mmol, 2.0 eq.) in small portions at 0° C. (ice bath). The resulting mixture was stirred for 48 h at 45° C. (oil bath) under an atmosphere of nitrogen followed by concentration under reduced pressure using a rotary evaporator. The residue was purified by silica gel column chromatography eluting with MeOH/DCM gradients (0~9%) to afford methyl 3-[(3R)-3-({6-[2-hydroxy-4-(trifluoromethyl)phenyl]-5-methylpyridazin-3-yl}amino)piperidin-1-yl]-2,2-dimethylpropanoate (150 mg, 37.7%) as a yellow solid after evaporation of the combined product containing fractions under reduced pressure using a rotary evaporator. LC/MS: MS (ESI) calcd, for C₂₃H₂₉F₃N₄O₃: 466.22. Found: 467.15 [M+H]⁺.

### Step 2: Synthesis of 3-[(3R)-3-({6-[2-hydroxy-4-(trifluoromethyl)phenyl]-5-methylpyridazin-3-yl}amino)piperidin-1-yl]-2,2-dimethylpropanoic acid lithium Salt

To a stirred solution of methyl 3-[(3R)-3-({6-[2-hydroxy-4-(trifluoromethyl)phenyl]-5-methylpyridazin-3-yl}amino)piperidin-1-yl]-2,2-dimethylpropanoate (100 mg, 0.2 mmol, 1.0 eq.) in MeOH (1 mL) and THF (5.0 mL) was added LiOH·H₂O (28 mg, 0.7 mmol, 3.1 eq.) in water (1 mL) dropwise at 0° C. (ice bath). The resulting mixture was stirred for 96 h at 50° C. The resulting mixture was concentrated under reduced pressure using a rotary evaporator to afford crude 3-[(3R)-3-({6-[2-hydroxy-4-(trifluoromethyl)phenyl]-5-methylpyridazin-3-yl}amino)piperidin-1-yl]-2,2-dimethylpropanoic acid lithium salt (110 mg, crude) as a yellow solid. The crude product was used directly in the next step without further purification, isolation, and characterization. LC/MS: MS (ESI) calcd, for C₂₂H₂₇F₃N₄O₃: 452.20. Found: 453.30 [M+H]⁺.

### Step 3: Synthesis of 3-[(3R)-3-({6-[2-Hydroxy-4-(trifluoromethyl)phenyl]-5-methylpyridazin-3-yl}amino)piperidin-1-yl]-1-(4-hydroxypiperidin-1-yl)-2,2-dimethylpropan-1-one (47)

A solution of crude 3-[(3R)-3-({6-[2-hydroxy-4-(trifluoromethyl)phenyl]-5-methylpyridazin-3-yl}amino)piperidin-1-yl]-2,2-dimethylpropanoic acid lithium salt (100 mg, 0.22 mmol, 1.0 eq.) and piperidin-4-ol (34 mg, 0.3 mmol, 1.5 eq.) in DMF (3 mL) was treated with DIEA (80 μL, 60 mg, 0.46 mmol, 2.1 eq.) and HOBt (30 mg, 0.2 mmol, 1.0 eq.) at 0° C. (ice bath) under an atmosphere of nitrogen followed by the addition of EDCI (64 mg, 0.3 mmol, 1.5 eq.) in portions at 0° C. (ice bath). The reaction mixture was stirred overnight at 50° C. (oil bath). The reaction was quenched by addition of an ice/water slurry (30 mL) at 0° C., and the resulting mixture was extracted with EA (3×15 mL). The combined organic extracts were washed with brine (2×30 mL) and dried over anhydrous Na₂SO₄. After filtration, the filtrate was concentrated under reduced pressure using a rotary evaporator. The crude product (110 mg) was purified by reparative RP-HPLC using the following conditions Column XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 35% B to 47% B in 10 min; Wavelengths: 254 nm/220 nm; RT1: 7.42 min to afford the title compound 3-[(3R)-3-({6-[2-hydroxy-4-(trifluoromethyl)phenyl]-5-methylpyridazin-3-yl}amino)piperidin-1-yl]-1-(4-hydroxypiperidin-1-yl)-2,2-dimethylpropan-1-one (47) (6 mg, 4.8%) as a white solid after lyophilization of the combined product containing fractions. LC/MS: MS (ESI) calcd, for C₂₇H₃₆F₃N₅O₃: 535.28. Found: 536.20 [M+H]⁺. ¹H NMR (300 MHz, DMSO-d₆): δ 7.39 (d, J=7.8 Hz, 1H), 7.21 (d, J=8.4 Hz, 1H), 7.15 (s, 1H), 6.81 (s, 1H), 4.15-4.00 (m, 3H), 3.90-3.80 (s, 1H), 3.30-3.10 (m, 2H), 2.90-2.80 (m, 1H), 2.70-2.55 (m, 3H), 2.50-2.30 (m, 2H), 2.14 (m, 3H), 1.95-1.70 (m, 4H), 1.68-1.40 (m, 4H), 1.30 (s, 6H) ppm. ¹⁹F NMR (282 MHz, DMSO-d₆): δ −64.29 ppm.

### Example 48: (R)-2-(3-((6-(2-Hydroxy-4-(trifluoromethyl)phenyl)-5-methylpyridazin-3-yl)amino)piperidin-1-yl)-1-(3-(hydroxymethyl)-3-methylazetidin-1-yl)ethan-1-one (48)

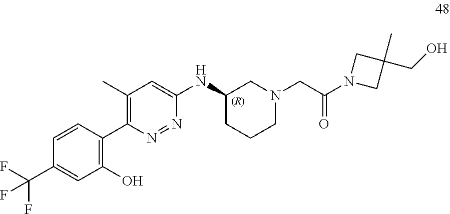

### Synthetic Route:

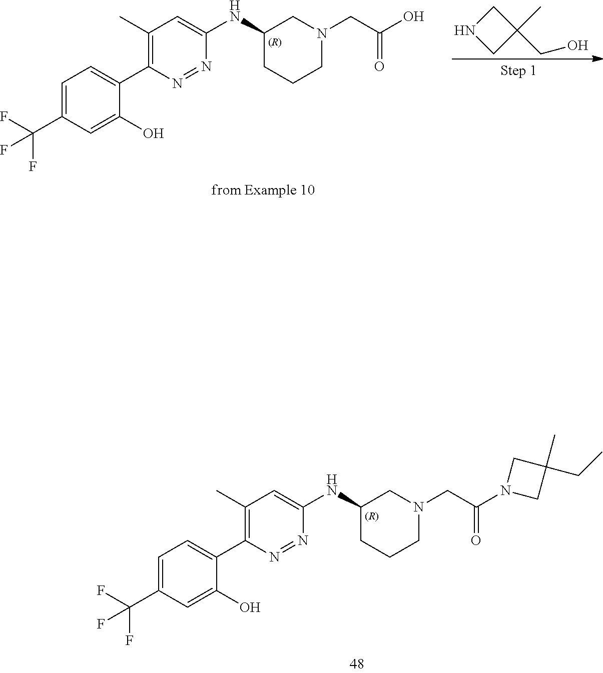

### Step 1: Synthesis of (R)-2-(3-((6-(2-Hydroxy-4-(trifluoromethyl)phenyl)-5-methylpyridazin-3-yl)amino)piperidin-1-yl)-1-(3-(hydroxymethyl)-3-methylazetidin-1-yl)ethan-1-one (48)

A colorless solution of [(3R)-3-({6-[2-hydroxy-4-(trifluoromethyl)phenyl]-5-methylpyridazin-3-yl}amino)piperidin-1-yl]acetic acid (prepared as disclosed in Example 10) (200 mg, 0.5 mmol, 1.0 eq.), EDCI (140 mg, 0.7 mmol, 1.5 eq.) and HOBt (66 mg, 0.5 mmol, 1.0 eq.) in DMF (2 mL) was treated with (3-methylazetidin-3-yl) methanol (49 mg, 0.5 mmol, 1.0 eq.) at 0° C. (ice bath) followed by dropwise addition of DIEA (255 μL, 189 mg, 1.5 mmol, 3.0 eq.). The reaction mixture solution was stirred overnight at rt. The reaction mixture was quenched by addition of an ice-water slurry (5 mL) and extracted with EtOAc (3×5 mL). The combined extracts were washed with H₂O (3×5 mL) and dried over anhydrous Na₂SO₄. After filtration, the filtrate was concentrated under reduced pressure using a rotary evaporator. The crude product (90 mg) was purified by preparative RP-HPLC using the following conditions Column: Xselect CSH Prep Fluoro-Phenyl Column, 19*250, 5 μm; Mobile Phase A: Water (10 mmol/L NH₄HCO₃), Mobile Phase B: MeOH; Flow rate: 25 mL/min; Gradient: 32% B to 57% B in 10 min, 57% B to 57% B in 16 min; Wavelengths: 254 nm/220 nm; RT1: 15.32 min to afford the target compound 2-[(3R)-3-({6-[2-hydroxy-4-(trifluoromethyl)phenyl]-5-methylpyridazin-3-yl}amino)piperidin-1-yl]-1-[3-(hydroxymethyl)-3-methylazetidin-1-yl]ethanone (48) (25 mg, 10.3%) as a white solid after lyophilization of the combined product containing fractions. LC/MS (ESI): mass calcd. for C₂₄H₃₀F₃N₅O₃: 493.23, found: m/z=494.20 [M+H]⁺. ¹H NMR (400 MHz, Methanol-d₄): δ 7.42 (d, J=8.0 Hz, 1H), 7.24 (d, J=7.6 Hz, 1H), 7.18 (s, 1H), 6.81 (s, 1H), 4.28-4.15 (m, 2H), 4.00-3.88 (m, 2H), 3.65-3.56 (m, 1H), 3.55-3.50 (m, 2H), 3.15-3.08 (m, 2H), 2.92-2.82 (m, 1H), 2.60-2.50 (m, 1H), 2.48-2.38 (m, 2H), 2.15 (s, 3H), 1.90-1.80 (m, 2H), 1.72-1.52 (m, 2H), 1.33-1.25 (m, 3H) ppm. ¹⁹F NMR (376 MHz, Methanol-d₄): δ −64.29 ppm.

### Example 49:5-(4-Methyl-6-{[(3R)-1-methylpiperidin-3-yl]amino}pyridazin-3-yl)-1-benzothiophen-4-ol (49)

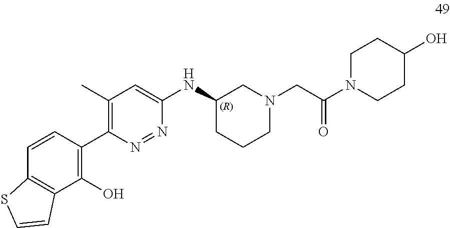

### Synthetic Route:

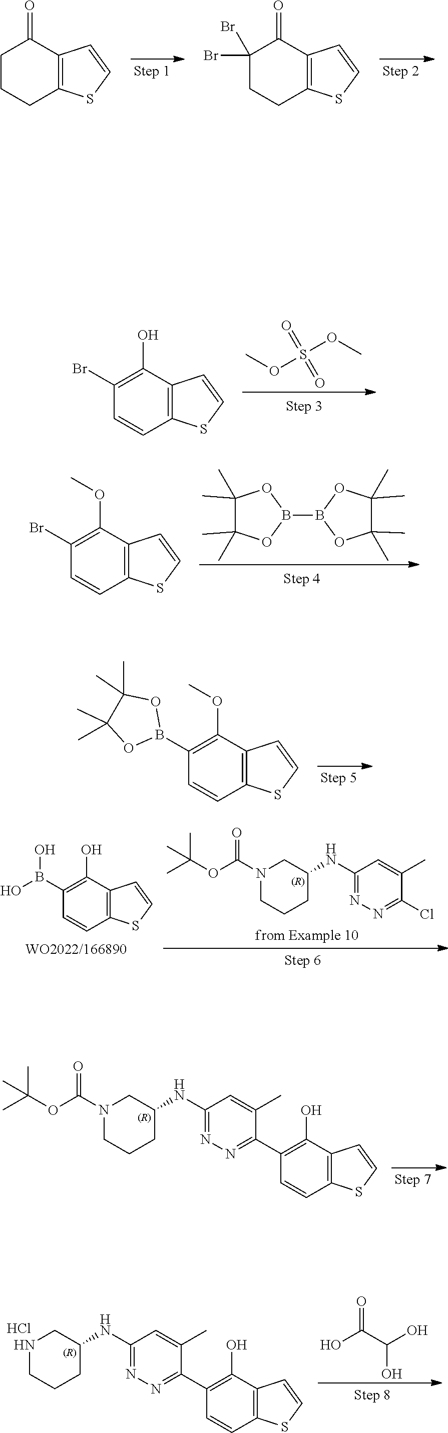

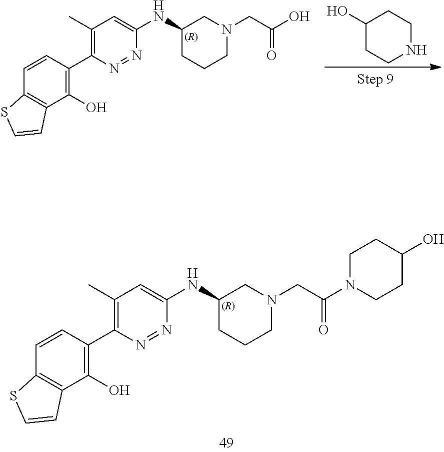

### Step 1: Synthesis of 5,5-dibromo-6,7-dihydro-1-benzothiophen-4-one)

A solution of CuBr₂ (58.7 g, 263 mmol, 4.0 eq.) in EtOAc (300 mL) was stirred at 80° C. (oil bath) for 2.5 h followed by dropwise addition (addition funnel) of a solution of 6,7-dihydro-5H-1-benzothiophen-4-one (10.0 g, 66 mmol, 1.0 eq.) in CHCl₃ (300 mL) at 80° C. The reaction mixture was stirred for an additional 16 h at 80° C. The resulting mixture was filtered, and the filter cake was washed with EtOAc (3×100 mL). The combined filtrates were washed with sat. NaHCO₃ (2×100 mL) and brine (1×100 mL). The organic phase was concentrated under reduced pressure using a rotary evaporator to afford 5,5-dibromo-6,7-dihydro-1-benzothiophen-4-one (17 g, crude) as a brown solid. The crude product was used directly in the next step without further purification, isolation and characterization. LC/MS (ESI): mass calcd, for C₈H₆Br₂OS: 309.85, found: m/z=310.75 [M+H]⁺.

### Step 2: Synthesis of 5,5-dibromo-6,7-dihydro-1-benzothiophen-4-one)

To a solution of 5,5-dibromo-6,7-dihydro-1-benzothiophen-4-one (16.0 g, 52 mmol, 1.0 eq.) in DMF (150 mL) was added Li₂CO₃ (22.6 g, 306 mmol, 5.9 eq.) in portions at rt. The reaction mixture was stirred for 6 h at 100° C. (oil bath). The reaction mixture was acidified to pH~1 with hydrochloric acid (1 M). The resulting mixture was extracted with EtOAc (3×250 mL). The combined organic extracts were washed with H₂O (3×200 mL). The organic solution was dried over anhydrous Na₂SO₄. After filtration, the filtrate was concentrated under reduced pressure using a rotary evaporator to afford 5-bromo-1-benzothiophen-4-ol (14 g, crude) as an off-white solid. The crude product was used directly in the next step without further purification, isolation and characterization. LC/MS (ESI): mass calcd, for C₈H₅BrOS: 227.92, found: m/z=226.90 [M−H]⁺.

### Step 3: Synthesis of 5-bromo-4-methoxy-1-benzothiophene

To a stirred mixture of 5-bromo-1-benzothiophen-4-ol (12.0 g, 52.4 mmol, 1.0 eq.) and dimethyl sulfate (8.0 g, 63.4 mmol, 1.2 eq.) in ACN (100 mL) were added K₂CO₃ (14.0 g, 101 mmol, 1.9 eq.). The reaction mixture was stirred for 15 h at 45° C. (oil bath). The resulting mixture was filtered, and the filter cake was washed with ACN (3×30 mL). The filtrate was concentrated under reduced pressure using a rotary evaporator. The residue was dissolved in MeOH (30 mL), then NaOH (3.0 g) was added and stirred at rt for an additional 1 h. The resulting mixture was filtered, and the filter cake was washed with MeOH (3×10 mL). The filtrate was concentrated under reduced pressure using a rotary evaporator. The mixture was dissolved in DCM (60 mL). The combined organic layers were washed with H₂O (3×30 mL) and dried over anhydrous Na₂SO₄. After filtration, the filtrate was concentrated under reduced pressure using a rotary evaporator. The residue was purified by silica gel column chromatography eluting with EA/PE gradients (0~9%) to afford 5-bromo-4-methoxy-1-benzothiophene (9.5 g, 74.6%) as a white solid after evaporation of the combined product containing fractions under reduced pressure using a rotary evaporator. LC/MS (ESI): mass calcd, for C₉H₇BrOS: 241.94, found: m/z=242.35 [M+H]⁺.

### Step 4: Synthesis of 2-(4-methoxy-1-benzothiophen-5-yl)-4,4,5,5-tetramethyl-1,3,2-dioxaborolane

To a stirred mixture of 5-bromo-4-methoxy-1-benzothiophene (8.5 g, 35.0 mmol, 1.0 eq.), bis(pinacolato)diboron (13.0 g, 51.2 mmol, 1.5 eq.) and Pd(PPh₃)₂Cl₂ (2.5 g, 3.5 mmol, 0.1 eq.) in 1,4-dioxane (100 mL) was dropwise added an aqueous solution of KOAc (2 M, 35 mL, 6.9 g, 70.3 mmol, 2.0 eq.) at rt. The resulting mixture was stirred for additional 16 h at 90° C. (oil bath). The resulting mixture was concentrated under reduced pressure using a rotary evaporator. The residue was purified by silica gel column chromatography eluting with EA/PE gradients (0~9%) to afford 2-(4-methoxy-1-benzothiophen-5-yl)-4,4,5,5-tetramethyl-1,3,2-dioxaborolane (5.5 g, 54.2%) as a white solid after evaporation of the combined product containing fractions under reduced pressure using a rotary evaporator. LC/MS (ESI): mass calcd, for C₁₅H₁₉BO₃S: 290.11, found: m/z=291.15 [M+H]⁺.

### Step 5: Synthesis of 4-hydroxy-1-benzothiophen-5-ylboronic Acid

To a stirred mixture of 2-(4-methoxy-1-benzothiophen-5-yl)-4,4,5,5-tetramethyl-1,3,2-dioxaborolane (5.5 g, 19.0 mmol, 1.0 eq.) in DCM (60 mL) was added BBr₃ (ca. 1.0 M in DCM, 38 mL, 402 mmol, 21.2 eq.) at 0° C. (ice bath). The resulting mixture was stirred overnight at rt. The reaction mixture was quenched by water and extraction of the aqueous phase with EtOAc (3×30 mL). The combined organic extracts were dried over anhydrous Na₂SO₄. After filtration, the filtrate was concentrated under reduced pressure using a rotary evaporator. The residue was purified by silica gel column chromatography eluting with MeOH/DCM gradients (0~9%) to afford 4-hydroxy-1-benzothiophen-5-ylboronic acid (1.5 g, 40.7%) as a yellow solid after evaporation of the combined product containing fractions under reduced pressure using a rotary evaporator. LC/MS (ESI): mass calcd, for C₈H₇BO₃S: 194.02, found: m/z=193.00 [M−H]⁺.

### Step 6: Synthesis of tert-butyl (3R)-3-{[6-(4-hydroxy-1-benzothiophen-5-yl)-5-methylpyridazin-3-yl]amino}piperidine-1-carboxylate

A solution of tert-butyl (3R)-3-[(6-chloro-5-methylpyridazin-3-yl)amino]piperidine-1-carboxylate (prepared as disclosed in Example 10) (1.5 g, 4.6 mmol, 1.0 eq) and 4-hydroxy-1-benzothiophen-5-ylboronic acid (prepared as disclosed in WO2022/166890 or Steps 1-5 above) (1.0 g, 5.1 mmol, 1.1 eq.) in 1,4-dioxane (10 mL) was treated with Pd(dppf)Cl₂·CH₂Cl₂ (336 mg, 0.5 mmol, 0.1 eq.) for 5 min at rt under an atmosphere of nitrogen followed by dropwise addition of a solution of NaHCO₃ (771 mg, 9.2 mmol, 2.0 eq.) in H₂O (10 mL) at rt under an atmosphere of nitrogen. The reaction mixture was stirred for 16 h at 100° C. (oil bath) under an atmosphere of nitrogen. The resulting mixture was concentrated under reduced pressure using a rotary evaporator. The residue was purified by silica gel column chromatography eluting with EA/PE gradients (0~50%) to afford tert-butyl (3R)-3-{[6-(4-hydroxy-1-benzothiophen-5-yl)-5-methylpyridazin-3-yl]amino}piperidine-1-carboxylate (580 mg, 28.6%) as a yellow solid after evaporation of the combined product containing fractions under reduced pressure using a rotary evaporator. LC/MS (ESI): mass calcd, for C₂₃H₂₈N₄O₃S: 440.19, found: m/z=441.25 [M+H]⁺.

### Step 7: Synthesis of 5-{4-methyl-6-[(3R)-piperidin-3-ylamino]pyridazin-3-yl}-1-benzothiophen-4-ol

To a stirred mixture of tert-butyl (3R)-3-{[6-(4-hydroxy-1-benzothiophen-5-yl)-5-methylpyridazin-3-yl]amino}piperidine-1-carboxylate (550.0 mg, 1.2 mmol, 1.0 eq.) in THF (6 mL) were added HCl (4 M in 1,4-dioxane, 3 mL, 12 mmol, 10 eq.) at 0° C. (ice bath). The resulting mixture was stirred for overnight at room temperature. The resulting mixture was concentrated under reduced pressure. This resulted in 5-{4-methyl-6-[(3R)-piperidin-3-ylamino]pyridazin-3-yl}-1-benzothiophen-4-ol (560 mg, crude) as a yellow solid. The crude product was used directly in the next step without further purification, isolation and characterization. LC/MS (ESI): mass calcd, for C₁₈H₂₀N₄OS: 340.14, found: m/z=341.25 [M+H]⁺.

### Step 8: Synthesis of [(3R)-3-{[6-(4-hydroxy-1-benzothiophen-5-yl)-5-methylpyridazin-3-yl]amino}piperidin-1-yl]acetic Acid

A mixture of 5-{4-methyl-6-[(3R)-piperidin-3-ylamino]pyridazin-3-yl}-1-benzothiophen-4-ol (400 mg, 1.2 mmol, 1.0 eq.) and dihydroxyacetic acid (216 mg, 2.4 mmol, 2.0 eq.) in MeOH (5 mL) was stirred for 5 min at 0° C. (ice bath) under an atmosphere of nitrogen. To the solution was added solid NaBH₃CN (148 mg, 2.4 mmol, 2.0 eq.) in small portions. The resulting mixture was stirred for 16 h at rt under an atmosphere of under nitrogen. The reaction was quenched through addition of an ice-water slurry (2 mL) at 0° C. The resulting mixture was concentrated under reduced pressure using a rotary evaporator. The residue was purified by reverse phase flash chromatography using the following conditions Column, Cat No: SO230120-2, C18, 120 g, 20~45 μm, 100 Å, Lot: BP0002P2503; mobile phase, CH₃CN:H₂O (0.05% NH₄HCO₃)=5% increased to CH₃CN:H₂O (0.05% NH₄HCO₃)=40% in 15 min, hold CH₃CN:H₂O (0.05% NH₄HCO₃)=40% in 10 min, up to CH₃CN:H₂O (0.05% NH₄HCO₃)=95% in 2 min, hold CH₃CN:H₂O (0.05% NH₄HCO₃)=95% in 10 min; UV detector wavelengths: 220 nm/254 nm to afford [(3R)-3-{[6-(4-hydroxy-1-benzothiophen-5-yl)-5-methylpyridazin-3-yl]amino}piperidin-1-yl]acetic acid (200 mg, 42.7%) as a yellow solid after lyophilization of the combined product containing fractions. LC/MS (ESI): mass calcd, for C₂₀H₂₂N₄O₃S: 398.48, found: m/z=399.10 [M+H]⁺.

### Step 9: Synthesis of 2-[(3R)-3-{[6-(4-hydroxy-1-benzothiophen-5-yl)-5-methylpyridazin-3-yl]amino}piperidin-1-yl]-1-(4-hydroxypiperidin-1-yl)ethanone (49)

To a solution of [(3R)-3-{[6-(4-hydroxy-1-benzothiophen-5-yl)-5-methylpyridazin-3-yl]amino}piperidin-1-yl]acetic acid (180 mg, 0.5 mmol, 1.0 eq.) in DMF (3 mL) was added DIEA (236 μL, 175 mg, 1.4 mmol, 3.0 eq.) for at 0° C. (ice bath) under an atmosphere of nitrogen followed by addition of EDCI (174 mg, 0.90 mmol, 2.0 eq.) in small portions at 0° C. (ice bath). The reaction was quenched through addition of an ice-water slurry (5 mL). The resulting mixture was extracted with EA (3×5 mL). The combined organic extracts were washed with H₂O (3×10 mL) and dried over anhydrous Na₂SO₄. After filtration, the filtrate was concentrated under reduced pressure using a rotary evaporator. The residue was purified by preparative TLC (DCM:MeOH=10:1, v/v) to afford a semi-purified product (80 mg). The semi-purified product was further purified by preparative RP-HPLC using the following conditions Column: XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 23% B to 53% B in 7 min; Wavelengths: 254 nm/220 nm; RT1: 5.62 min to afford the target compound 2-[(3R)-3-{[6-(4-hydroxy-1-benzothiophen-5-yl)-5-methylpyridazin-3-yl]amino}piperidin-1-yl]-1-(4-hydroxypiperidin-1-yl)ethanone (49) (10 mg, 4.5%) as a white solid after lyophilization of the combined product containing fractions. LC/MS (ESI): mass calcd, for C₂₀H₂₂N₄O₃S: 481.21, found: m/z=482.25 [M+H]⁺. ¹H NMR (300 MHz, Methanol-d₄): δ 7.58-7.56 (m, 1H), 7.55-7.46 (m, 2H), 7.30-7.20 (m, 1H), 6.90-6.86 (m, 1H), 4.20-3.70 (m, 4H), 3.30-3.25 (m, 3H), 3.10-2.90 (m, 1H), 2.70-2.55 (m, 1H), 2.40-2.10 (m, 5H), 2.02-1.80 (m, 5H), 1.78-1.60 (m, 1H), 1.58-1.35 (m, 3H) ppm.

### Example 50: (R)-2-(3-((6-(4-Hydroxybenzo[b]thiophen-5-yl)-4,5-dimethylpyridazin-3-yl)amino)piperidin-1-yl)-1-(4-hydroxypiperidin-1-yl)ethan-1-one (50)

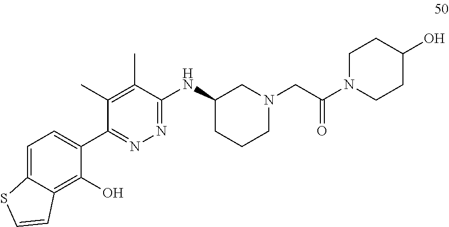 [SMILES: Cc1c(N[C@@H]2CCCN(CC(=O)N3CCC(O)CC3)C2)nnc(-c2ccc3sccc3c2O)c1C]

### Synthetic Route:

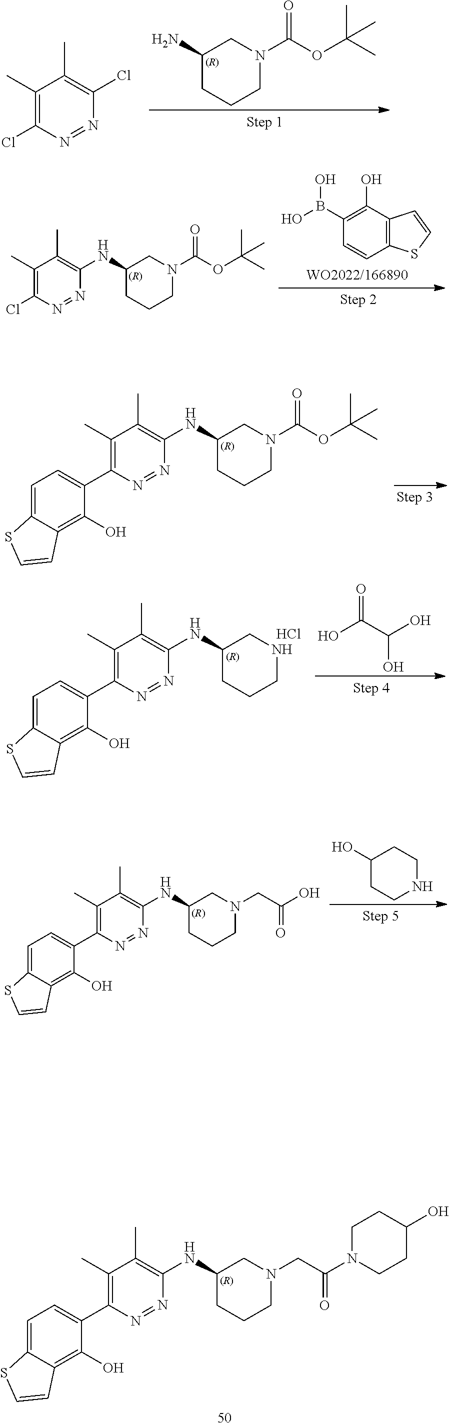

### Step 1: Synthesis of tert-butyl (3R)-3-[(6-chloro-4,5-dimethylpyridazin-3-yl)amino]piperidine-1-carboxylate

A solution of 3,6-dichloro-4,5-dimethylpyridazine (2.0 g, 11.2 mmol, 1.0 eq.), tert-butyl (3R)-3-aminopiperidine-1-carboxylate (4.5 g, 22.5 mmol, 2.0 eq.) and DIEA (3.9 mL, 2.89 g, 22.5 mmol, 2.0 eq.) in NMP (20 mL) was stirred at 150° C. (oil bath) for 18 h. The reaction was monitored by LCMS and upon completion, the reaction was quenched by addition of a water/ice slurry (40 mL) and extracted with EtOAc (3×20 mL). The combined organic extracts were washed with water (3×20 mL) and brine (1×50 mL), then dried over anhydrous Na₂SO₄. After filtration, the filtrate was concentrated under reduced pressure using a rotary evaporator. The residue was purified by silica gel column chromatography eluting with EA/PE gradients (0~50%) to afford tert-butyl tert-butyl (3R)-3-[(6-chloro-4,5-dimethylpyridazin-3-yl)amino]piperidine-1-carboxylate (3.6 g, 93.4%) as a yellow oil after evaporation of the combined product containing fractions under reduced pressure using a rotary evaporator. LC/MS: MS (ESI) calcd for C₁₆H₂₅ClN₄O₂: 340.16. Found: m/z=341.20 [M+H]⁺.

### Step 2: Synthesis of tert-butyl (3R)-3-{[6-(4-hydroxy-1-benzothiophen-5-yl)-4,5-dimethylpyridazin-3-yl]amino}piperidine-1-carboxylate

To a solution of tert-butyl (3R)-3-[(6-chloro-4,5-dimethylpyridazin-3-yl)amino]piperidine-1-carboxylate (700 mg, 2.0 mmol, 1.0 eq.) in a mixture of 1,4-dioxane (5 mL) and H₂O (5 mL) were added 4-hydroxy-1-benzothiophen-5-yl boronic acid (prepared according to WO2022/166890) (598 mg, 3.0 mmol, 1.5 eq.), Pd(dppf)Cl₂CH₂Cl₂ (335 mg, 0.4 mmol, 0.2 eq.) and NaHCO₃ (345 mg, 4.1 mmol, 2.0 eq.). The reaction mixture was stirred for 16 h at 100° C. (oil bath) in a sealed reaction tube under an atmosphere of nitrogen. The reaction mixture was concentrated under reduced pressure using a rotary evaporator. The residue was purified by silica gel column chromatography eluting with EA/PE gradients (0~27%) to afford tert-butyl (3R)-3-{[6-(4-hydroxy-1-benzothiophen-5-yl)-4,5-dimethylpyridazin-3-yl]amino}piperidine-1-carboxylate (800 mg, 77.9%) as a yellow solid after evaporation of the combined product containing fractions under reduced pressure using a rotary evaporator. LC/MS (ESI): mass calcd, for C₂₄H₃₀N₄O₃S: 454.20, found: m/z=455.20 [M+H]⁺.

### Step 3: Synthesis of methyl 5-{4,5-dimethyl-6-[(3R)-piperidin-3-ylamino]pyridazin-3-yl}-1-benzothiophen-4-ol) hydrochloride

To a stirred solution of tert-butyl (3R)-3-{[6-(4-hydroxy-1-benzothiophen-5-yl)-4,5-dimethylpyridazin-3-yl]amino}piperidine-1-carboxylate (800 mg, 1.76 mmol, 1.0 eq.) in 1,4-dioxane (8 mL) was added dropwise HCl (4 M in dioxane, 3 mL, 12 mmol, ~7 eq.) at rt. The resulting mixture was stirred for 1 h at rt before the reaction mixture was concentrated under reduced pressure using a rotary evaporator to afford methyl 5-{4,5-dimethyl-6-[(3R)-piperidin-3-ylamino]pyridazin-3-yl}-1-benzothiophen-4-ol) hydrochloride (700 mg, crude) as an off-white solid. The crude product was used directly in the next step without further purification, isolation and characterization. LC/MS (ESI): mass calcd, for C₁₉H₂₂N₄OS: 354.15, found: m/z=355.10 [M+H]⁺

### Step 4: Synthesis of methyl[(3R)-3-{[6-(4-hydroxy-1-benzothiophen-5-yl)-4,5-dimethylpyridazin-3-yl]amino}piperidin-1-yl]acetic Acid

To a stirred solution of 5-{4,5-dimethyl-6-[(3R)-piperidin-3-ylamino]pyridazin-3-yl}-1-benzothiophen-4-ol hydrochloride (700 mg, 1.9 mmol, 1.0 eq.) in MeOH (10 mL) were added dihydroxyacetic acid (182 mg, 1.9 mmol, 1.0 eq.) and NaBH₃CN (248.2 mg, 3.9 mmol, 2.0 eq.) in small portions at 0° C. (ice bath). The resulting reaction mixture was stirred for 6 h at rt. The reaction was quenched through an ice-water slurry (5 mL) and was concentrated under reduced pressure using a rotary evaporator. The residue was directly purified by reverse phase column chromatography using the following conditions: Column C18 silica gel; mobile phase, ACN in water (0.05% NH₄HCO₃), 10% to 40% gradient in 20 min; UV detector wavelength: 254 nm to afford methyl[(3R)-3-{[6-(4-hydroxy-1-benzothiophen-5-yl)-4,5-dimethylpyridazin-3-yl]amino}piperidin-1-yl]acetic acid (190 mg, 20.9%) as a yellow solid after evaporation of the combined product containing fractions under reduced pressure using a rotary evaporator. LC/MS (ESI): mass calcd, for C₂₆H₃₃N₅O₃S: 495.23, found: m/z=496.15 [M+H]⁺.

### Step 5: Synthesis of (R)-2-(3-((6-(4-hydroxybenzo[b]thiophen-5-yl)-4,5-dimethylpyridazin-3-yl)amino)piperidin-1-yl)-1-(4-hydroxypiperidin-1-yl)ethan-1-one (50)

A yellow solution of [(3R)-3-{[6-(4-hydroxy-1-benzothiophen-5-yl)-4,5-dimethylpyridazin-3-yl]amino}piperidin-1-yl]acetic acid (170 mg, 0.4 mmol, 1.0 eq.), EDCI (119 mg, 0.6 mmol, 1.5 eq.) and HOBt (56 mg, 0.4 mmol, 1.0 eq.) in DMF (2.0 mL) was treated with piperidin-4-ol (42 mg, 0.4 mmol, 1.0 eq.) followed by dropwise addition of DIEA (216 μL, 160 mg, 1.2 mmol, 3.0 eq.) at 0° C. (ice bath). The reaction mixture was stirred for 6 h with gradual warming to rt. The resulting mixture was concentrated under reduced pressure using a rotary evaporator. The crude product (100 mg) was purified by preparative RP-HPLC using the following conditions Column: XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 25% B to 55% B in 7 min; Wavelengths: 254 nm/220 nm; RT1: 6.07 min to afford the target compound 2-[(3R)-3-{[6-(4-hydroxy-1-benzothiophen-5-yl)-4,5-dimethylpyridazin-3-yl]amino}piperidin-1-yl]-1-(4-hydroxypiperidin-1-yl)ethanone (50) (53 mg, 25.3%) as a yellow solid after lyophilization of the combined product containing fractions. LC/MS (ESI): mass calcd, for C₂₆H₃₃N₅O₃S: 495.23, found: m/z=496.15 [M+H]⁺. ¹H NMR (300 MHz, Methanol-d₄): δ 7.62-7.56 (m, 1H), 7.55-7.45 (m, 2H), 7.22-7.12 (m, 1H), 4.42-4.30 (m, 1H), 4.25-4.15 (m, 1H), 4.10-3.95 (m, 1H), 3.90-3.80 (m, 1H), 3.80-3.65 (m, 1H), 3.50-3.40 (m, 1H), 3.30-3.20 (m, 1H), 3.15-3.10 (m, 1H), 3.00-2.85 (m, 1H), 2.75-2.55 (m, 1H), 2.45-2.20 (m, 2H), 2.15-2.10 (m, 3H), 2.19-2.16 (m, 3H), 2.00-1.78 (m, 5H), 1.75-1.60 (m, 2H), 1.58-1.38 (m, 1H) ppm.

### Example 51:5-(6-(((R)-1-(((1s,3S)-3-Hydroxycyclobutyl)methyl)piperidin-3-yl)amino)-4-methylpyridazin-3-yl)benzo[b]thiophen-4-ol (51)

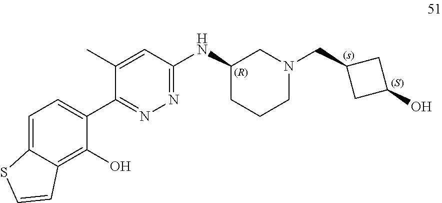

### Synthetic Route:

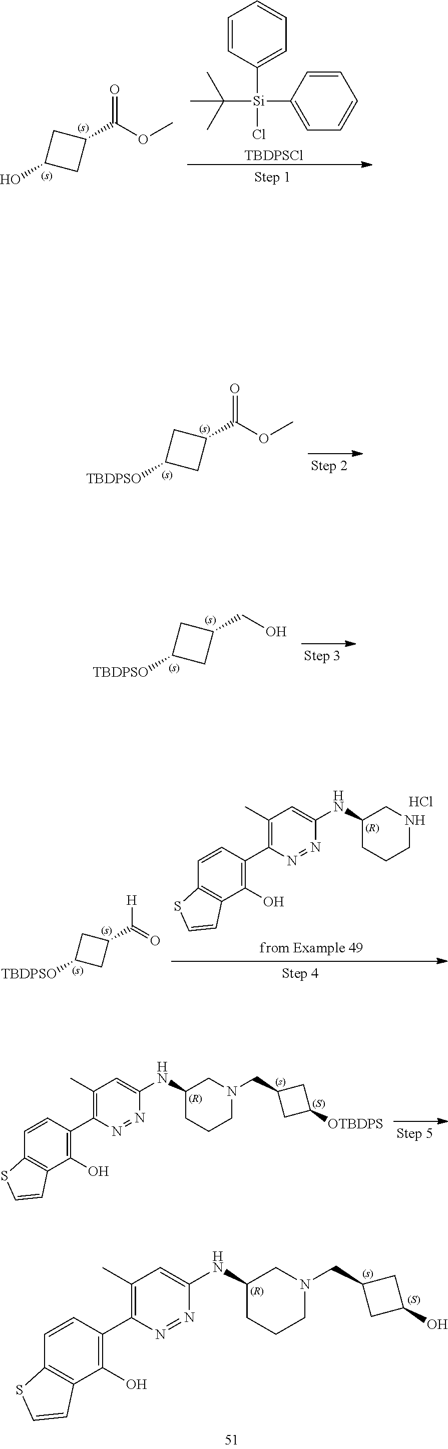

### Step 1: Synthesis of methyl (1s,3s)-3-((tert-butyldiphenylsilyl)oxy)cyclobutane-1-carboxylate

Methyl (1s,3s)-3-hydroxycyclobutane-1-carboxylate (300 mg, 2.3 mmol, 1.0 eq.) was dissolved in 5 mL of DCM, and then tert-butyldiphenylchlorosilane (697 mg, 2.5 mmol, 1.1 eq) and 1H-imidazole (345 mg, 5.1 mmol, 2.2 eq.) were added at 0° C. (ice bath). The reaction mixture was then filtered through a Celite® cake and washed with 5 mL of DCM. The combined filtrates were evaporated under reduced pressure and the residue was subjected to flash column chromatography using 9% EtOAc in PE to afford methyl (1s,3s)-3-((tert-butyldiphenylsilyl)oxy)cyclobutane-1-carboxylate (800 mg, 93%) as a colorless oil. ¹H NMR (400 MHz, Chloroform-d): δ 7.67-7.57 (m, 4H), 7.43-7.31 (m, 6H), 4.14-4.03 (m, 1H), 3.68-3.59 (m, 3H), 2.33 (s, 5H), 1.01 (s, 9H) ppm.

### Step 2 Synthesis of ((1s,3s)-3-((tert-butyldiphenylsilyl)oxy)cyclobutyl)methanol

To a solution of methyl (1s,3s)-3-((tert-butyldiphenylsilyl)oxy)cyclobutane-1-carboxylate (470 mg, 1.2 mmol, 1.0 eq.) in 10 mL of THF, LiBH₄ (2.5 mL, 1.0 M in THF, 2.5 mmol, 2.0 eq.) was added slowly at rt. The reaction mixture was stirred overnight at rt. The reaction mixture was cooled to 0° C. in an ice-water bath and extracted with 3×15 mL of EtOAc. The combined organic extracts were washed with 20 mL of saturated aqueous NH₄Cl solution, dried over anhydrous sodium sulfate, filtered, and concentrated by rotary evaporation under reduced pressure. The residue was subjected to flash column chromatography using 6% EtOAc in PE to obtain ((1s,3s)-3-((tert-butyldiphenylsilyl)oxy)cyclobutyl)methanol (430 mg, 96%) as a colorless oil. ¹H NMR (400 MHz, Methanol-d₄): δ 7.66-7.61 (m, 4H), 7.42-7.33 (m, 6H), 4.11 (t, J=7.5 Hz, 1H), 3.45 (d, J=4.4 Hz, 2H), 2.22-2.14 (m, 2H), 1.80-1.70 (m, 3H), 1.00-0.98 (m, 9H) ppm.

### Step 3: Synthesis of (1s,3s)-3-((tert-butyldiphenylsilyl)oxy)cyclobutane-1-carbaldehyde

To a solution of ((1s,3s)-3-((tert-butyldiphenylsilyl)oxy)cyclobutyl)methanol (430 mg, 1.2 mmol, 1.0 eq.) in 10 mL of DCM, PCC (410 mg, 1.9 mmol, 1.5 eq.) was added at 0° C. (ice bath). The reaction mixture was stirred at rt for 3 hours. Without evaporation of solvents, the reaction crude was directly loaded onto a flash chromatography column using 100% DCM (isocratic) to afford (1s,3s)-3-((tert-butyldiphenylsilyl)oxy)cyclobutane-1-carbaldehyde (300 mg, 66%) as a colorless oily substance. ¹H NMR (400 MHz, Chloroform-d): δ 9.62 (d, J=2.8 Hz, 1H), 7.66-7.61 (m, 4H), 7.43-7.34 (m, 6H), 4.26-4.19 (m, 1H), 2.53-2.43 (m, 1H), 2.35-2.18 (m, 4H), 1.02 (s, 9H) ppm.

### Step 4: Synthesis of 5-(6-(((R)-1-(((1s,3S)-3-((tert-butyldiphenylsilyl)oxy)cyclobutyl)methyl)piperidin-3-yl)amino)-4-methylpyridazin-3-yl)benzo[b]thiophen-4-ol

In a single-necked flask, (R)-5-(4-methyl-6-(piperidin-3-ylamino)pyridazin-3-yl)benzo[b]thiophen-4-ol hydrochloride (prepared as disclosed in Example 49) (170 mg, 0.5 mmol, 1.0 eq.) and (1s,3s)-3-((tert-butyldiphenylsilyl)oxy)cyclobutane-1-carbaldehyde (338 mg, 1.0 mmol, 2.0 eq.) and were dissolved in 5 mL of MeOH followed by addition of acetic acid (171 μL, 180 mg, 3.0 mmol, 6.0 eq.) under an atmosphere of nitrogen. The reaction mixture was stirred at rt for 30 min. Sodium cyanoborohydride (94 mg, 1.5 mmol, 3.0 eq.) was added at 0° C. (ice bath), and the reaction mixture was stirred with gradual warming to rt for 2 h. Upon completion, the reaction mixture was quenched with 20 mL of saturated aqueous NaHCO₃ solution at 0° C. and extracted with 3×50 mL of EtOAc. The combined organic extracts were dried over anhydrous sodium sulfate. After filtration, the filtrate was concentrated by rotary evaporation under reduced pressure. The residue was purified by flash column chromatography (0-9% MeOH in DCM) to afford 5-(6-(((R)-1-(((1s,3S)-3-((tert-butyldiphenylsilyl)oxy)cyclobutyl)methyl)piperidin-3-yl)amino)-4-methylpyridazin-3-yl)benzo[b]thiophen-4-ol (200 mg, 60%) as a yellow solid. LC/MS: mass calcd for C₃₉H₄₆N₄O₂SSi 662.31, found m/z=663.05 [M+H]⁺. ¹H NMR (400 MHz, Chloroform-d): δ 7.65-7.55 (m, 5H), 7.47-7.43 (m, 2H), 7.40-7.31 (m, 6H), 7.21 (d, J=8.3 Hz, 1H), 6.78 (s, 1H), 4.15-4.04 (m, 2H), 3.62 (br s, 1H), 3.19-3.09 (m, 1H), 2.82-2.70 (m, 1H), 2.61-2.56 (m, 2H), 2.35-2.25 (m, 3H), 2.17 (s, 3H), 2.08-1.93 (m, 1H), 1.91-1.76 (m, 2H), 1.75-1.65 (m, 3H), 1.45-1.30 (m, 2H), 0.98 (s, 9H) ppm.

### Step 5: Synthesis of 5-(6-(((R)-1-(((1s,3S)-3-hydroxycyclobutyl)methyl)piperidin-3-yl)amino)-4-methylpyridazin-3-yl)benzo[b]thiophen-4-ol (51)

In a single-necked flask, 5-(6-(((R)-1-(((1s,3S)-3-((tert-butyldiphenylsilyl)oxy)cyclobutyl)methyl)piperidin-3-yl)amino)-4-methylpyridazin-3-yl)benzo[b]thiophen-4-ol (200 mg, 0.3 mmol, 1.0 eq.) was dissolved in 10 mL of THF followed by addition of tetrabutylammonium fluoride (300 μL, 2.0 M in THF, 0.6 mmol, 2.0 eq.) under an atmosphere of nitrogen. The reaction mixture was stirred at rt overnight. Upon completion, the reaction mixture was diluted with 50 mL of EtOAc and washed with 3×50 mL of saturated aqueous NaHCO₃ solution and dried over anhydrous sodium sulfate. After filtration, the filtrate was concentrated by rotary evaporation under reduced pressure. The residue was purified by flash column chromatography (0-8% MeOH in DCM) to afford the target compound (51) (51 mg, 40%) as a yellow solid. LC/MS: mass calcd for C₂₃H₂₈N₄O₂S 424.19, found m/z=425.00 [M+H]⁺. ¹H NMR (400 MHz, Methanol-d₄): δ 7.59 (d, J=5.6 Hz, 1H), 7.49 (d, J=6.4 Hz, 2H), 7.25 (d, J=8.2 Hz, 1H), 6.81 (s, 1H), 4.19-4.00 (m, 2H), 3.22-3.11 (m, 1H), 2.86-2.76 (m, 1H), 2.59 (d, J=6.4 Hz, 2H), 2.51-2.40 (m, 2H), 2.37-2.27 (m, 1H), 2.20 (s, 3H), 2.19-2.07 (m, 1H), 2.07-1.98 (m, 2H), 1.89-1.79 (m, 1H), 1.78-1.66 (m, 1H), 1.64-1.52 (m, 2H), 1.50-1.39 (m, 1H) ppm.

### Example 52: 5-(6-(((R)-1-(((1r,3R)-3-Hydroxycyclobutyl)methyl)piperidin-3-yl)amino)-4-methylpyridazin-3-yl)benzo[b]thiophen-4-ol (52)

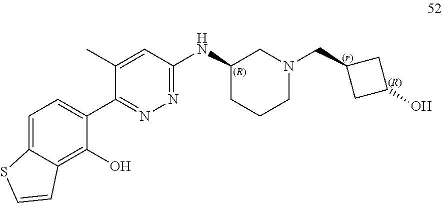

### Synthetic Route:

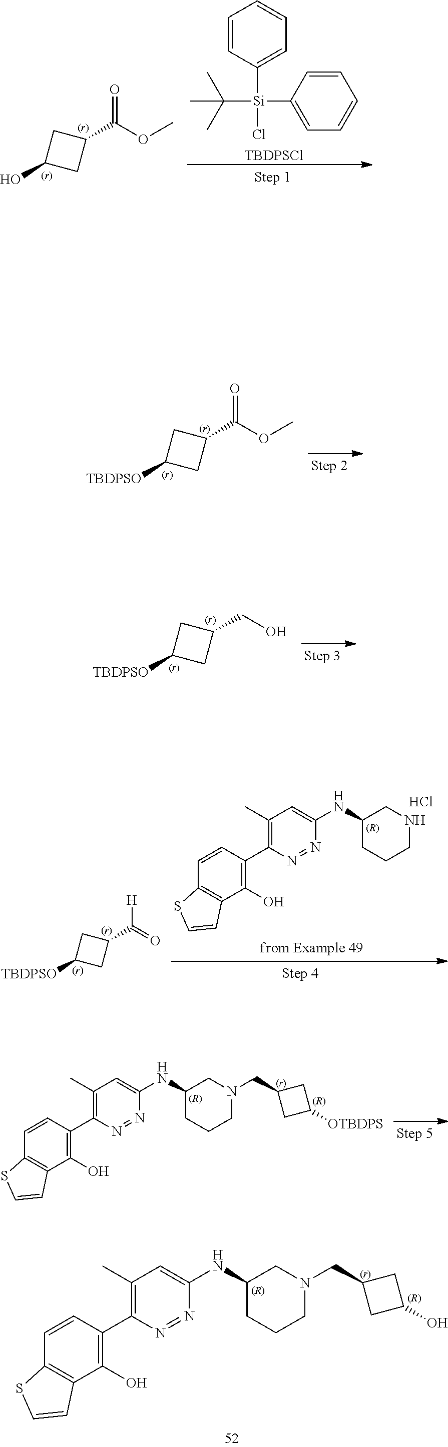

### Step 1: Synthesis of methyl (1r,3r)-3-((tert-Butyldiphenylsilyl)oxy)cyclobutane-1-carboxylate

In a single-necked flask, methyl (1r,3r)-3-hydroxycyclobutane-1-carboxylate (400 mg, 3.0 mmol, 1.0 eq.) was dissolved in 5 mL of DCM followed by addition of TBDPSCl (1.3 g, 4.6 mmol, 1.5 eq.) and 1H-imidazole (460 mg, 6.8 mmol, 2.2 eq.) under an atmosphere of nitrogen at 0° C. (ice bath). The reaction mixture was stirred at rt overnight. Upon completion, the reaction mixture was filtered through a Celite® cake. The filtrate was concentrated by rotary evaporation under reduced pressure. The residue was purified by flash column chromatography (2% EA in PE) to afford methyl (1r,3r)-3-((tert-butyldiphenylsilyl)oxy)cyclobutane-1-carboxylate (1.1 g, 93%) as a colorless oil. ¹H NMR (400 MHz, Chloroform-d): δ 7.63-7.60 (m, 4H), 7.41-7.33 (m, 6H), 4.58-4.49 (m, 1H), 3.60 (s, 3H), 3.01-2.93 (m, 1H), 2.44-2.38 (m, 2H), 2.36-2.29 (m, 2H), 1.02 (s, 9H) ppm.

### Step 2: Synthesis of ((1r,3r)-3-((tert-butyldiphenylsilyl)oxy)cyclobutyl)methanol

In a single-necked flask, methyl (1r,3r)-3-((tert-butyldiphenylsilyl)oxy)cyclobutane-1-carboxylate (1.4 g, 3.6 mmol, 1.0 eq.) was dissolved in 15 mL of THF followed by addition of LiBH₄ (7.3 mL, 7.2 mmol, 2.0 eq.) under an atmosphere of nitrogen at 0° C. (ice bath). The reaction mixture was stirred at rt overnight. Upon completion, the reaction mixture was quenched with 50 mL of iced water and extracted with 3×50 mL of EtOAc. The combined organic extracts were washed with 3×50 mL of saturated aqueous NH₄Cl solution and dried over anhydrous sodium sulfate. After filtration, the filtrate was concentrated by rotary evaporation under reduced pressure. The residue was purified by flash column chromatography (6% EtOAc in PE) to afford ((1r,3r)-3-((tert-butyldiphenylsilyl)oxy)cyclobutyl)methanol (1.2 g, 96%) as a colorless oil. ¹H NMR (400 MHz, Chloroform-d): δ 7.65 (d, J=7.7 Hz, 4H), 7.45-7.34 (m, 6H), 4.42-4.33 (m, 1H), 3.49 (d, J=7.2 Hz, 2H), 2.38-2.28 (m, 1H), 2.27-2.18 (m, 2H), 2.05-1.97 (m, 2H), 1.03 (s, 9H) ppm.

### Step 3: Synthesis of (1r,3r)-3-((tert-butyldiphenylsilyl)oxy)cyclobutane-1-carbaldehyde

In a single-necked flask, ((1r,3r)-3-((tert-butyldiphenylsilyl)oxy)cyclobutyl)methanol (500 mg, 1.5 mmol, 1.0 eq.) was dissolved in 10 mL of DCM followed by addition of PCC (474 mg, 2.2 mmol, 1.5 eq.) under an atmosphere of nitrogen at 0° C. (ice bath). The reaction mixture was stirred at rt for 3 h. Upon completion, the reaction mixture was directly purified by flash column chromatography (isocratic) (100% DCM) to afford (1r,3r)-3-((tert-butyldiphenylsilyl)oxy)cyclobutane-1-carbaldehyde (330 mg, 66%) as a colorless oil. ¹H NMR (400 MHz, Chloroform-d): δ 9.69 (s, 1H), 7.62 (d, J=7.9 Hz, 4H), 7.45-7.33 (m, 6H), 4.38-4.29 (m, 1H), 3.04-2.94 (m, 1H), 2.50-2.41 (m, 2H), 2.36-2.27 (m, 2H), 1.04 (s, 9H) ppm.

### Step 4: Synthesis of 5-(6-(((R)-1-(((1r,3R)-3-((tert-butyldiphenylsilyl)oxy)cyclobutyl)methyl)piperidin-3-yl)amino)-4-methylpyridazin-3-yl)benzo[b]thiophen-4-ol

To a solution of (1r,3r)-3-((tert-butyldiphenylsilyl)oxy)cyclobutane-1-carbaldehyde (338 mg, 1.0 mmol, 2.0 eq.) and (R)-5-(4-methyl-6-(piperidin-3-ylamino)pyridazin-3-yl)benzo[b]thiophen-4-ol (prepared as disclosed in Example 49) (170 mg, 0.5 mmol, 1.0 eq.) in 5 mL of MeOH, 4 drops of acetic acid were added. The mixture was stirred at rt for 30 minutes. Then, it was cooled to 0° C. (ice bath) and solid NaBH₃CN (63 mg, 1.0 mmol, 2.0 eq.) was added in portions. The mixture was stirred at rt overnight. After MS monitoring confirmed the complete consumption of the starting material, the mixture was cooled to 0° C. (ice bath), and 2 mL of water was added dropwise to quench the reaction. The mixture was then extracted with 3×20 mL of EtOAc, and the combined organic extracts were washed with 20 mL of saturated aqueous NaHCO₃ solution, dried over anhydrous sodium sulfate, filtered, and concentrated by rotary evaporator under reduced pressure. The sample was purified by flash column chromatography (4% MeOH in DCM) to yield 5-(6-(((R)-1-(((1r,3R)-3-((tert-butyldiphenylsilyl)oxy)cyclobutyl)methyl)piperidin-3-yl)amino)-4-methylpyridazin-3-yl)benzo[b]thiophen-4-ol (190 mg, 57%) as a yellow solid. LC/MS: mass calcd for C₃₉H₄₆N₄O₂SSi 662.31, found m/z=663.30, [M+H]⁺. ¹H NMR (400 MHz, Methanol-d₄): δ 7.61 (d, J=7.5 Hz, 8H), 7.57 (d, J=5.6 Hz, 1H), 7.49-7.44 (m, 2H), 7.42-7.31 (m, 2H), 7.20 (d, J=8.2 Hz, 1H), 6.78 (s, 1H), 4.17-4.05 (m, 2H), 2.89-2.75 (m, 2H), 2.66 (d, J=7.2 Hz, 2H), 2.54 (s, 2H), 2.43-2.21 (m, 8H), 2.17 (s, 3H), 2.06-1.93 (m, 3H), 1.82 (s, 2H), 1.77-1.62 (m, 3H), 0.99 (d, J=3.4 Hz, 9H) ppm.

### Step 5: Synthesis of 5-(6-(((R)-1-(((1r,3R)-3-Hydroxycyclobutyl)methyl)piperidin-3-yl)amino)-4-methylpyridazin-3-yl)benzo[b]thiophen-4-ol (52)

To a solution of 5-(6-(((R)-1-(((1r,3R)-3-((tert-butyldiphenylsilyl)oxy)cyclobutyl)methyl)piperidin-3-yl)amino)-4-methylpyridazin-3-yl)benzo[b]thiophen-4-ol (190 mg, 0.3 mmol, 1.0 eq.) in 5 mL of THF, TBAF (150 mg, 0.6 mmol, 2.0 eq.) was added. The flask was then stirred at rt overnight. After MS monitoring confirmed the completion of the reaction, the reaction mixture was diluted with 20 mL of a saturated aqueous NaHCO₃ solution, and then extracted with 2×20 mL of EtOAc. The combined organic extracts were dried over anhydrous sodium sulfate, filtered, and concentrated by rotary evaporator under reduced pressure. The residue was purified by flash column chromatography (8% MeOH in DCM) to afford the target compound 5-(6-(((R)-1-(((1r,3R)-3-hydroxycyclobutyl)methyl)piperidin-3-yl)amino)-4-methylpyridazin-3-yl)benzo[b]thiophen-4-ol (52) (61 mg, 50%) as a yellow solid. LC/MS: mass calcd for C₂₃H₂₈N₄O₂S 424.19, found m/z=425.15, [M+H]⁺. ¹H NMR (400 MHz, Methanol-d₄): δ 7.58 (d, J=5.6 Hz, 1H), 7.48 (d, J=6.0 Hz, 2H), 7.24 (d, J=8.3 Hz, 1H), 6.80 (s, 1H), 4.31-4.01 (m, 2H), 3.18-3.10 (m, 1H), 2.77 (br s, 1H), 2.59-2.55 (m, 2H), 2.45 (p, J=6.4 Hz, 1H), 2.33-2.24 (m, 1H), 2.20 (s, 3H), 2.12-2.06 (m, 2H), 2.06-1.96 (m, 2H), 1.87-1.78 (m, 1H), 1.76-1.66 (m, 1H), 1.61-1.53 (m, 1H), 1.49-1.38 (m, 1H) ppm.

### Example 53: (R)-2-(6-((1-(4-Hydroxycyclohexyl)piperidin-3-yl)amino)-4-methylpyridazin-3-yl)-5-(trifluoromethyl)phenol (Fraction A) (53)

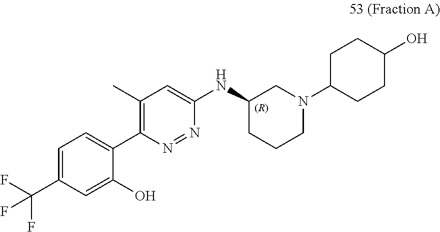

### Synthetic Route:

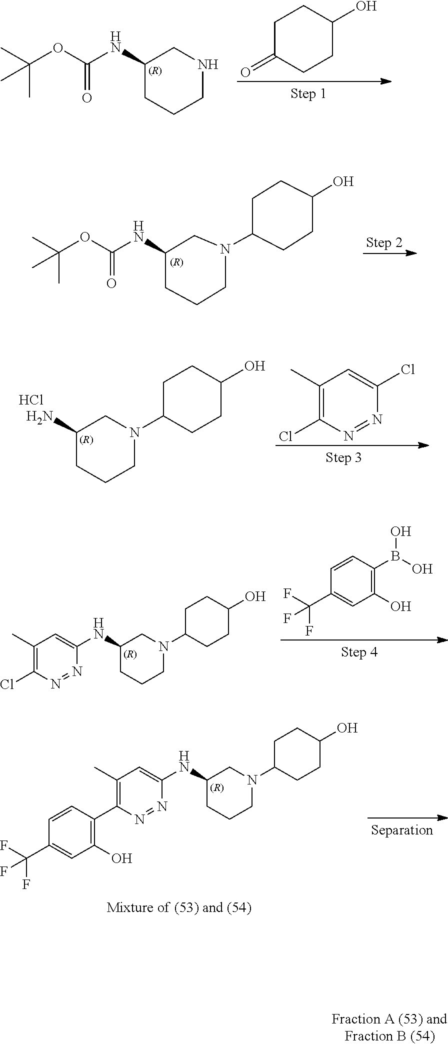

### Step 1: Synthesis of tert-butyl (R)-(1-(4-hydroxycyclohexyl)piperidin-3-yl)carbamate

To a solution of tert-butyl N-[(3R)-piperidin-3-yl]carbamate (2.0 g, 10.0 mmol 1.0 eq.) and 4-hydroxycyclohexan-1-one (2.28 g, 20.0 mmol, 2.0 eq.) in MeOH (30 mL) was added NaBH₃CN (942 mg, 15.0 mmol, 1.5 eq.). The resulting solution was stirred at rt for 4 h. The reaction mixture was quenched and washed with a saturated aqueous NH₄Cl solution (30 mL) and extracted with EtOAc (3×80 mL). The combined organic extracts were dried over Na₂SO₄ and concentrated under reduced pressure using a rotary evaporator to afford tert-butyl N-[(3R)-1-(4-hydroxycyclohexyl)piperidin-3-yl]carbamate (1.5 g, 42.8% yield) as a yellow oil which was used directly in the next step without further isolation, purification, or characterization. LC/MS: mass calcd for: C₁₆H₃₀N₂O₃: 298.23, found: m/z=299.15 [M+H]⁺.

### Step 2: Synthesis of (R)-4-(3-aminopiperidin-1-yl)cyclohexan-1-ol hydrochloride

To a solution of tert-butyl N-[(3R)-1-(4-hydroxycyclohexyl)piperidin-3-yl]carbamate (1.4 g, 4.7 mmol) in DCM (5 mL) was added HCl (5 mL) 0° C. (ice bath). The resulting solution was stirred at rt for 2 h. The reaction mixture was concentrated under reduced pressure using a rotary evaporator to afford 4-[(3R)-3-aminopiperidin-1-yl]cyclohexan-1-ol hydrochloride as a white solid (1.2 g, 87.2% yield). LC/MS: mass calcd for C₁₁H₂₂N₂O: 198.17, found: m/z=199.05 [M+H]⁺.

### Step 3: Synthesis of (R)-4-(3-((6-chloro-5-methylpyridazin-3-yl)amino)piperidin-1-yl)cyclohexan-1-ol

To solution of 4-[(3R)-3-aminopiperidin-1-yl]cyclohexan-1-ol hydrochloride (1.2 g, 5.4 mmol, 1.1 eq.) and DIEA (2.56 mL, 1.90 g, 14.7 mmol, 3.0 eq.) in NMP (4 mL) was added 3,6-dichloro-4-methylpyridazine (800 mg, 4.91 mmol, 1.0 eq.). The reaction mixture was stirred at 150° C. overnight (sealed tube). The reaction mixture was submitted to preparative RP column chromatography (0~60% MeCN/water+0.05% NH₄HCO₃)) to afford a brown oil (500 mg) after lyophilization of the combined product containing fractions. The obtained oil was submitted to purification by silica gel column chromatography eluting with PE/EtOAc (0~100%) to afford 4-[(3R)-3-[(6-chloro-5-methylpyridazin-3-yl)amino]piperidin-1-yl]cyclohexan-1-ol as a yellow oil (100 mg, 5.02% yield) after evaporation of the combined product containing fractions under reduced pressure using a rotary evaporator. LC/MS: mass calcd for C₁₆H₂₅ClN₄O: 324.17, found: m/z=325.15 [M+H]⁺.

### Step 4: Synthesis of (R)-2-(6-((1-(4-hydroxycyclohexyl)piperidin-3-yl)amino)-4-methylpyridazin-3-yl)-5-(trifluoromethyl)phenol

To a solution of 4-[(3R)-3-[(6-chloro-5-methylpyridazin-3-yl)amino]piperidin-1-yl]cyclohexan-1-ol (100 mg, 0.31 mmol) in 1,4-dioxane/H₂O (v/v, 2/1, 1.5 mL/0.75 mL) were added 2-hydroxy-4-(trifluoromethyl)phenylboronic acid (102 mg, 0.49 mmol, 1.6 eq.), Pd(PPh₃)₄ (11 mg, 0.009 mmol, 0.03 eq.) and NaHCO₃ (78 mg, 0.924 mmol, 3.0 eq.). The resulting solution was stirred at 150° C. under an atmosphere of nitrogen (sealed tube) for 1 h. After cooling down to rt, the reaction mixture was concentrated under reduced pressure using a rotary evaporator. The residue was submitted to purification by silica gel column chromatography eluting with DCM/MeOH (0~20%) to afford a yellow oil (75 mg). The obtained oil was submitted to preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 25 mL/min; Gradient: 20% B to 45% B in 7 min; Wavelength: 254 nm; RT1: 6.32 min to afford the target compound 2-(6-{[(3R)-1-(4-hydroxycyclohexyl)piperidin-3-yl]amino}-4-methylpyridazin-3-yl)-5-(trifluoromethyl)phenol as a mixture of diastereomers as a white solid (24.4 mg, 17.6% yield). LC/MS: mass calcd for C₂₃H₂₉F₃N₄O₂: 450.22, found: m/z=451.15 [M+H]⁺. ¹H NMR (400 MHz, DMSO-d₆): δ 10.52 (s, 1H), 7.41-7.38 (m, 1H), 7.23-7.20 (m, 2H), 6.68 (s, 1H), 6.59-6.57 (m, 1H), 4.48-4.25 (m, 1H), 3.96-3.73 (m, 2H), 3.05-2.98 (m, 1H), 2.68-2.65 (m, 1H), 2.34-2.25 (m, 2H), 2.18-2.06 (m, 1H), 2.05-2.00 (m, 3H), 1.94-1.78 (m, 2H), 1.77-1.67 (m, 4H), 1.59-1.49 (m, 1H), 1.43-1.24 (m, 4H), 1.20-1.04 (m, 1H) ppm. ¹⁹F NMR (376 MHz, DMSO-d₆): δ −61.16 ppm.

The purified material 2-(6-{[(3R)-1-(4-hydroxycyclohexyl)piperidin-3-yl]amino}-4-methylpyridazin-3-yl)-5-(trifluoromethyl)phenol, a mixture of diastereomeric compounds, was separated into two fractions, Fraction A and Fraction B (without absolute stereochemical assignment).

Fraction A: LC/MS: mass calcd for C₂₃H₂₉F₃N₄O₂ 450.22, found m/z=451.10 [M+H]⁺. ¹H NMR (400 MHz, Methanol-d₄): δ 7.37 (d, J=8.0 Hz, 1H), 7.20-7.18 (m, 1H), 7.14 (s, 1H), 6.75 (s, 1H), 4.56 (s, 1H), 4.10 (m, 1H), 3.89 (s, 1H), 3.22-3.20 (m, 1H), 2.86-2.83 (m, 1H), 2.48 (t, J=10.8 Hz, 2H), 2.34 (t, J=10.8 Hz, 1H), 2.11 (s, 3H), 2.02 (m, 1H), 1.85-1.64 (m, 8H), 1.52-1.27 (m, 3H) ppm. ¹⁹F NMR (376 MHz, Methanol-d₄): δ −64.35 ppm.

### Example 54: (R)-2-(6-((1-(4-Hydroxycyclohexyl)piperidin-3-yl)amino)-4-methylpyridazin-3-yl)-5-(trifluoromethyl)phenol (Fraction B) (54)

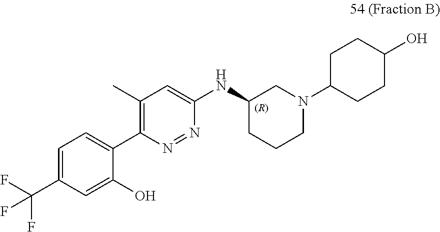

Fraction B: LC/MS: mass calcd for C₂₃H₂₉F₃N₄O₂ 450.22, found m/z=451.10 [M+H]⁺. ¹H NMR (400 MHz, Methanol-d₄): δ 7.37 (d, J=8.0 Hz, 1H), 7.19 (d, J=8.0 Hz, 1H), 7.14 (s, 1H), 6.74 (s, 1H), 4.56 (s, 1H), 4.07-4.04 (m, 1H), 3.48-3.42 (m, 1H), 3.16-3.13 (m, 1H), 2.80-2.77 (m, 1H), 2.38 (t, J=9.6 Hz, 2H), 2.24 (t, J=9.6 Hz, 1H), 2.11 (s, 3H), 1.99-1.80 (m, 5H), 1.79-1.76 (m, 1H), 1.67-1.64 (m, 1H), 1.41-1.24 (m, 5H) ppm. ¹⁹F NMR (376 MHz, Methanol-d₄): δ −64.33 ppm.

### Example 55: (R)-2-(3-((6-(2-Hydroxy-4-(trifluoromethyl)phenyl)-5-methylpyridazin-3-yl)amino)piperidin-1-yl)-1-(4-hydroxy-4-methylpiperidin-1-yl)ethan-1-one (55)

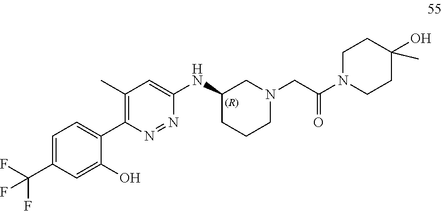

### Synthetic Route:

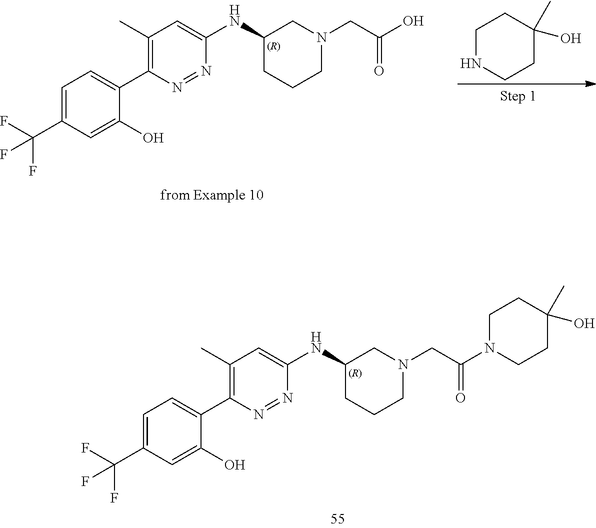

### Step 1: Synthesis of 2-[(3R)-3-({6-[2-hydroxy-4-(trifluoromethyl)phenyl]-5-methylpyridazin-3-yl}amino)piperidin-1-yl]-1-(4-hydroxy-4-methylpiperidin-1-yl)ethanone (55)

To a stirred mixture of [(3R)-3-({6-[2-hydroxy-4-(trifluoromethyl)phenyl]-5-methylpyridazin-3-yl}amino)piperidin-1-yl]acetic acid (prepared as disclosed for Example 10) (120 mg, 0.3 mmol, 1.0 eq.) and 4-methylpiperidin-4-ol (41 mg, 0.4 mmol, 1.2 eq.) in pyridine (4 mL) was added EDCI (85 mg, 0.4 mmol, 1.5 eq.) at rt. The reaction mixture was stirred for an additional 16 h at 50° C. (oil bath). The resulting mixture was concentrated under reduced pressure using a rotary evaporator. The crude product (100 mg) was purified by preparative RP-HPLC using the following conditions Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min; Wavelengths: 254 nm/220 nm; RT1: 5.57 min) to afford the target compound 2-[(3R)-3-({6-[2-hydroxy-4-(trifluoromethyl)phenyl]-5-methylpyridazin-3-yl}amino)piperidin-1-yl]-1-(4-hydroxy-4-methylpiperidin-1-yl)ethanone (55) (18.3 mg, 12.2%) as a light yellow solid after lyophilization of the combined product containing fractions. LC/MS: MS (ESI) calcd for C₂₅H₃₂F₃N₅O₃: 507.25. Found: m/z=508.25 [M+H]⁺. ¹H NMR (400 MHz, Methanol-d₄): δ 7.40-7.39 (m, 1H), 7.24 (d, J=8.0, 1H), 7.18 (s, 1H), 6.79 (s, 1H), 4.22-4.12 (m, 1H), 4.10-4.05 (m, 0.5H), 3.90-3.80 (m, 1H), 3.78-3.70 (m, 0.5H), 3.67-3.42 (m, 1H), 3.30-3.15 (m, 2H), 3.05-2.80 (m, 1H), 2.70-2.55 (m, 1H), 2.40-2.20 (m, 2H), 2.20-2.10 (m, 3H), 2.00-1.79 (m, 3H), 1.75-1.45 (m, 6H), 1.20-1.10 (m, 3H) ppm. ¹⁹F NMR (376 MHz, Methanol-d₄): δ −64.28 ppm.

### Example 56: (R)-2-(3-((4-(4-Hydroxybenzo[b]thiophen-5-yl)-5,7-dihydrofuro[3,4-d]pyridazin-1-yl)amino)piperidin-1-yl)-1-(4-hydroxypiperidin-1-yl)ethan-1-one (56)

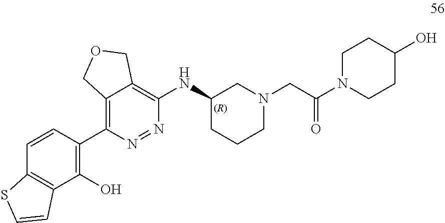

### Synthetic Route:

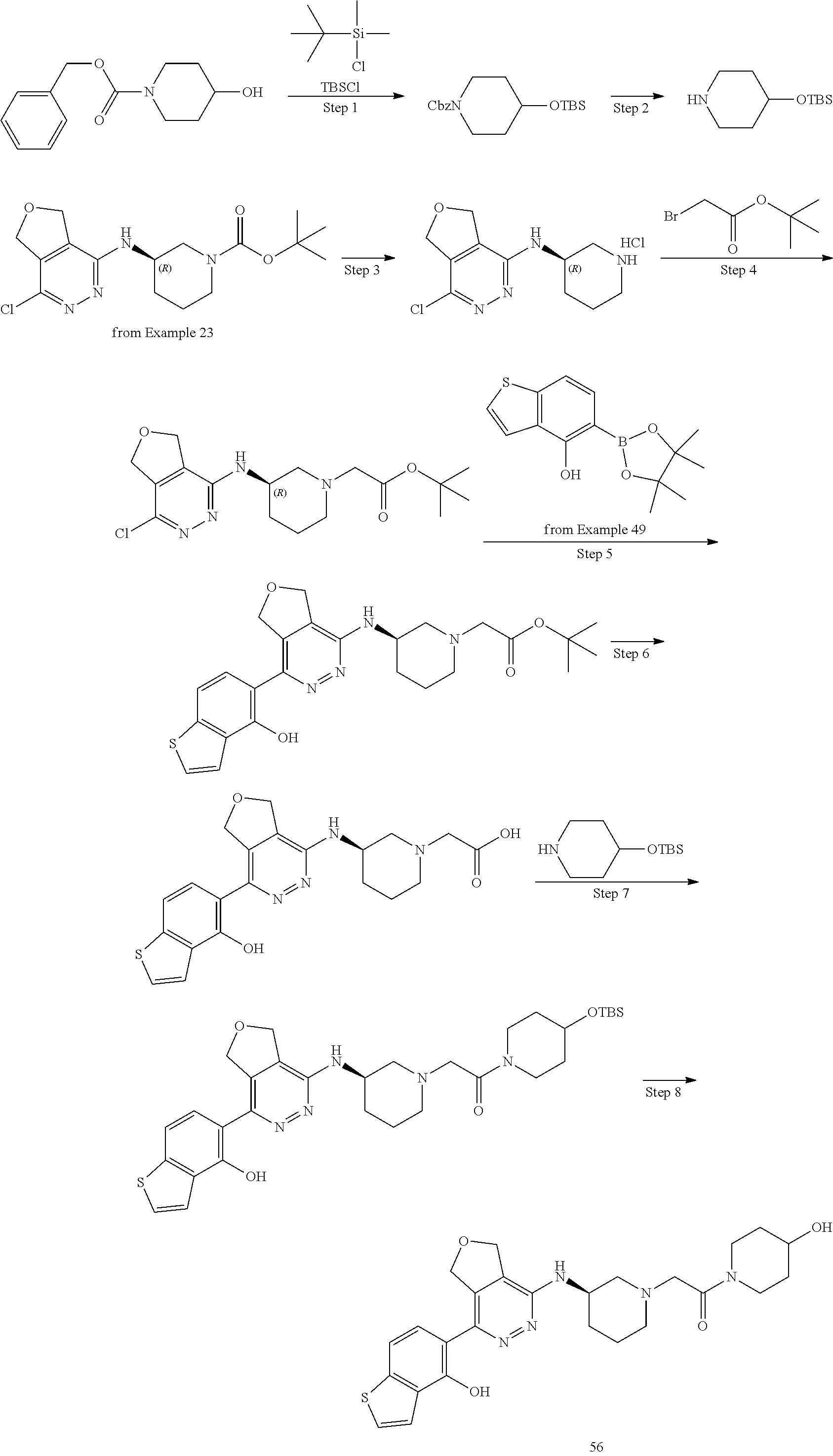

### Step 1: Synthesis of benzyl 4-((tert-butyldimethylsilyl)oxy)piperidine-1-carboxylate

To a solution of benzyl 4-hydroxypiperidine-1-carboxylate (2.0 g, 8.5 mmol, 1.0 eq.) in 30 mL of DMF, imidazole (1.2 g, 7.3 mmol, 2.0 eq.) and TBSCl (2.6 g, 17.3 mmol, 2.0 eq.) were added to the flask, and the reaction was stirred at 40° C. (heating block) for 16 hours. After the completion of the reaction, 60 mL of water was added into the mixture, and the solution was extracted with 3×20 mL of EtOAc. The combined organic extracts were dried over sodium sulfate, filtered, and concentrated by rotary evaporation under reduced pressure. The crude residue was purified with flash column chromatography (100% DCM) (isocratic) to afford benzyl 4-((tert-butyldimethylsilyl)oxy)piperidine-1-carboxylate (3.0 g, quant.) as a yellow oil after evaporation of the combined product containing fractions under reduced pressure using a rotary evaporator. LC/MS: mass calcd for C₁₉H₃₁NO₃Si 349.21, found m/z=350.15, [M+H]⁺. ¹H NMR (400 MHz, Chloroform-d): δ 7.37-7.29 (m, 5H), 5.12 (s, 2H), 3.90 (hept, J=3.4 Hz, 1H), 3.73-3.62 (m, 2H), 3.46-3.34 (m, 2H), 1.76-1.64 (m, 2H), 1.56-1.44 (m, 2H), 0.89 (s, 9H), 0.05 (s, 6H) ppm.

### Step 2: Synthesis of 4-((tert-butyldimethylsilyl)oxy)piperidine

To a solution of benzyl 4-((tert-butyldimethylsilyl)oxy)piperidine-1-carboxylate (1.0 g, 2.9 mmol, 1.0 eq.) in 20 mL of MeOH, 10 wt. % dry basis Pd/C (106 mg, 0.1 mmol, 0.03 eq.) was added. The flask was purged with hydrogen three times, and the reaction was stirred under an atmosphere of hydrogen (1 atm) at rt for 3 hours. The solution was filtered through a Celite® cake and concentrated by rotary evaporation under reduced pressure. The crude residue was purified by flash column chromatography (10% MeOH in DCM) to afford 4-((tert-butyldimethylsilyl)oxy)piperidine (300 mg, 49%) as a yellow oil after evaporation of the combined product containing fraction under reduced pressure using a rotary evaporator. ¹H NMR (400 MHz, Methanol-d₄): δ 3.89-3.76 (m, 1H), 3.05-2.96 (m, 2H), 2.69-2.55 (m, 2H), 1.83-1.70 (m, 2H), 1.54-1.39 (m, 2H), 0.88 (d, J=2.0 Hz, 9H), 0.06 (s, 6H) ppm.

### Step 3: Synthesis of (R)-4-chloro-N-(piperidin-3-yl)-5,7-dihydrofuro[3,4-d]pyridazin-1-amine hydrochloride

In a single-necked flask, tert-butyl (R)-3-((4-chloro-5,7-dihydrofuro[3,4-d]pyridazin-1-yl)amino)piperidine-1-carboxylate (prepared as disclosed for Example 23) (640 mg, 1.8 mmol, 1.0 eq.) was dissolved in HCl (10 mL, 4 M in 1,4-dioxane, 40 mmol, ~22 eq.) and stirred at rt for 1 h. Upon completion, the reaction mixture was concentrated by rotary evaporation under reduced pressure to afford (R)-4-chloro-N-(piperidin-3-yl)-5,7-dihydrofuro[3,4-d]pyridazin-1-amine hydrochloride (510 mg, 75%) as a yellow solid. The material was used directly in the next step without further isolation or purification. LC/MS: mass calcd for C₁₁H₁₅ClN₄O 254.09, found 254.95, m/z=[M+H]⁺. ¹H NMR (400 MHz, Methanol-d₄): δ 5.25-5.13 (m, 4H), 4.40-4.26 (m, 1H), 3.61 (d, J=11.8 Hz, 1H), 3.40-3.34 (m, 1H), 3.14-2.99 (m, 2H), 2.24-2.14 (m, 1H), 2.14-2.04 (m, 1H), 1.98-1.86 (m, 1H), 1.84-1.73 (m, 1H) ppm.

### Step 4: Synthesis of tert-butyl (R)-2-(3-((4-chloro-5,7-dihydrofuro[3,4-d]pyridazin-1-yl)amino)piperidin-1-yl)acetate

In a single-necked flask, (R)-4-chloro-N-(piperidin-3-yl)-5,7-dihydrofuro[3,4-d]pyridazin-1-amine hydrochloride (400 mg, 1.6 mmol, 1.0 eq.) was dissolved in 20 mL of ACN, to which tert-butyl 2-bromoacetate (337 mg, 1.7 mmol, 1.1 eq.) and K₂CO₃ (651 mg, 4.7 mmol, 3.0 eq.) were added. This mixture was heated at 80° C. overnight (heating block). The reaction mixture was cooled to room temperature, filtered through a Celite® cake, and concentrated by rotary evaporation under reduced pressure. The crude product was purified by flash column chromatography (2% MeOH in DCM) to obtain tert-butyl (R)-2-(3-((4-chloro-5,7-dihydrofuro[3,4-d]pyridazin-1-yl)amino)piperidin-1-yl)acetate (280 mg, 48%) as a yellow oily substance after evaporation of the combined product containing fractions under reduced pressure using a rotary evaporator. LC/MS: mass calcd for C₁₇H₂₅ClN₄O₃ 368.16, found m/z=369.20, [M+H]⁺. ¹H NMR (400 MHz, Chloroform-d): δ 5.52 (d, J=6.2 Hz, 1H), 5.12 (t, J=4.1 Hz, 2H), 5.09 (t, J=3.1 Hz, 2H), 4.46-4.38 (m, 1H), 3.10 (q, J=10.2 Hz, 2H), 2.77 (d, J=11.6 Hz, 2H), 2.56 (d, J=11.0 Hz, 1H), 2.40-2.33 (m, 1H), 2.03-1.93 (m, 1H), 1.58-1.48 (m, 2H), 1.45 (s, 9H) ppm.

### Step 5: Synthesis of tert-butyl (R)-2-(3-((4-(4-hydroxybenzo[b]thiophen-5-yl)-5,7-dihydrofuro[3,4-d]pyridazin-1-yl)amino)piperidin-1-yl)acetate

In a sealed tube, tert-butyl (R)-2-(3-((4-chloro-5,7-dihydrofuro[3,4-d]pyridazin-1-yl)amino)piperidin-1-yl)acetate (280 mg, 0.8 mmol, 1.0 eq.) was dissolved in 12 mL of 1,4-dioxane, followed by the addition of 5-(4,4,5,5-tetramethyl-1,3,2-dioxaborolan-2-yl)benzo[b]thiophen-4-ol (prepared as disclosed in Example 49) (220 mg, 0.8 mmol, 1.0 eq.), Na₂CO₃ (242 mg, 2.3 mmol, 3.0 eq.), Pd(PPh₃)₄ (88 mg, 0.1 mmol, 0.1 eq.), and 3 mL of H₂O, in sequence. The mixture was purged with nitrogen, sealed, and heated at 110° C. overnight. The reaction mixture was cooled to room temperature, quenched with 30 mL of water and extracted with 3×10 mL of EtOAc. The combined organic extracts were dried over anhydrous sodium sulfate, filtered, and concentrated by rotary evaporation under reduced pressure. The crude product was purified by flash column chromatography (2% MeOH/DCM) to afford tert-butyl (R)-2-(3-((4-(4-hydroxybenzo[b]thiophen-5-yl)-5,7-dihydrofuro[3,4-d]pyridazin-1-yl)amino)piperidin-1-yl)acetate (360 mg, 96%) as a yellow solid after evaporation of the combined product containing fractions under reduced pressure using a rotary evaporator. LC/MS: mass calcd for C₂₅H₃₀N₄O₄S 482.20, found m/z=483.30, [M+H]⁺.

### Step 6: Synthesis of (R)-2-(3-((4-(4-hydroxybenzo[b]thiophen-5-yl)-5,7-dihydrofuro[3,4-d]pyridazin-1-yl)amino)piperidin-1-yl)acetic Acid

tert-butyl (R)-2-(3-((4-(4-hydroxybenzo[b]thiophen-5-yl)-5,7-dihydrofuro[3,4-d]pyridazin-1-yl)amino)piperidin-1-yl)acetate (350 mg, 0.7 mmol, 1.0 eq.) was dissolved in HCl in 1,4-dioxane (4.0 M, 2 mL, 8.0 mmol, 11.4 eq.), and the mixture was stirred at rt for 16 hours. The solution was evaporated to dryness by rotary evaporation under reduced pressure. The residue was purified by flash column chromatography (5% MeOH in DCM) to give (R)-2-(3-((4-(4-hydroxybenzo[b]thiophen-5-yl)-5,7-dihydrofuro[3,4-d]pyridazin-1-yl)amino)piperidin-1-yl)acetic acid (270 mg, 84%) as a yellow solid after evaporation of the combined product containing fractions under reduced pressure using a rotary evaporator. LC/MS: mass calcd for C₂₁H₂₂N₄O₄S 426.14, found m/z=427.30, [M+H]⁺.

### Step 7: Synthesis of (R)-1-(4-((tert-butyldimethylsilyl)oxy) piperidin-1-yl)-2-(3-((4-(4-hydroxybenzo[b]thiophen-5-yl)-5,7-dihydrofuro[3,4-d]pyridazin-1-yl)amino)piperidin-1-yl)ethan-1-one

To a mixture of (R)-2-(3-((4-(4-hydroxybenzo[b]thiophen-5-yl)-5,7-dihydrofuro[3,4-d]pyridazin-1-yl)amino)piperidin-1-yl)acetic acid (250 mg, 0.6 mmol, 1.0 eq.), HOBt hydrate (water ~12%, 175 mg, 1.2 mmol, 2.0 eq.), 4-(tert-butyldimethylsilyl)oxy)piperidine (122 mg, 0.6 mmol, 1.0 eq.), and EDCI (220 mg, 1.2 mmol, 2.0 eq.) in 6 mL of DMF, DIPEA (396 μL, 294 mg, 2.4 mmol, 4.0 eq.) were added. The reaction mixture was allowed to stir at rt for 16 hours, and then all solvents were evaporated by rotary evaporation under reduced pressure. The residue was purified by flash column chromatography (3% MeOH in DCM) to afford (R)-1-(4-((tert-butyldimethylsilyl)oxy) piperidin-1-yl)-2-(3-((4-(4-hydroxybenzo[b]thiophen-5-yl)-5,7-dihydrofuro[3,4-d]pyridazin-1-yl)amino)piperidin-1-yl)ethan-1-one (300 mg, 82%) as a yellow oily substance after evaporation of the combined product containing fractions under reduced pressure using a rotary evaporator. LC/MS: mass calcd for C₃₂H₄₅N₅O₄SSi 623.30, found m/z=624.20, [M+H]⁺.

### Step 8: Synthesis of (R)-2-(3-((4-(4-hydroxybenzo[b]thiophen-5-yl)-5,7-dihydrofuro[3,4-d]pyridazin-1-yl)amino)piperidin-1-yl)-1-(4-hydroxypiperidin-1-yl)ethan-1-one (56)

To a solution of (R)-1-(4-((tert-butyldimethylsilyl)oxy) piperidin-1-yl)-2-(3-((4-(4-hydroxybenzo[b]thiophen-5-yl)-5,7-dihydrofuro[3,4-d]pyridazin-1-yl)amino)piperidin-1-yl)ethan-1-one (250 mg, 0.4 mmol, 1.0 eq.) in 2.5 mL of 1,4-dioxane, HCl in 1,4-dioxane (4.0 M, 2.5 mL, 10 mmol, 25.0 eq.) was added, and the solution was allowed to stir at rt for 2 hours. All solvents were evaporated by rotary evaporation under reduced pressure, and the crude residue was purified by preparative RP-HPLC (5-65% ACN in water with 0.1% HCOOH), and the pure fractions were collected to afford the target compound (R)-2-(3-((4-(4-hydroxybenzo[b]thiophen-5-yl)-5,7-dihydrofuro[3,4-d]pyridazin-1-yl)amino)piperidin-1-yl)-1-(4-hydroxypiperidin-1-yl)ethan-1-one (56) (28 mg, 11%) as a yellow solid after lyophilization of the combined product containing fractions. LC/MS: mass calcd for C₂₆H₃₁N₅O₄S 509.21, found m/z=510.30, [M+H]⁺. ¹H NMR (400 MHz, DMSO-d₆): δ 7.62 (d, J=5.5 Hz, 1H), 7.55 (t, J=4.5 Hz, 1H), 7.47 (d, J=8.5 Hz, 1H), 7.16 (d, J=8.7 Hz, 1H), 6.77-6.59 (m, 1H), 5.45-5.33 (m, 2H), 4.95 (s, 2H), 4.72 (br s, 1H), 4.17 (br s, 1H), 3.91-3.58 (m, 3H), 3.27-2.87 (m, 6H), 2.78-2.61 (m, 1H), 2.08-1.99 (m, 1H), 1.98-1.82 (m, 2H), 1.79-1.15 (m, 6H) ppm.

### Example 57: (R)-2-(3-((4-(2-Hydroxy-4-(trifluoromethyl)phenyl)-6,7-dihydro-5H-pyrrolo[3,4-d]pyridazin-1-yl)amino)piperidin-1-yl)-1-(4-hydroxypiperidin-1-yl)ethan-1-one (57)

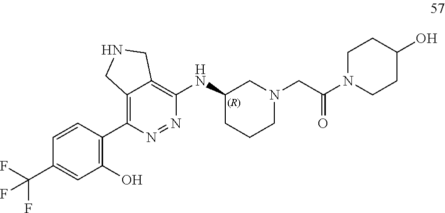

### Synthetic Route:

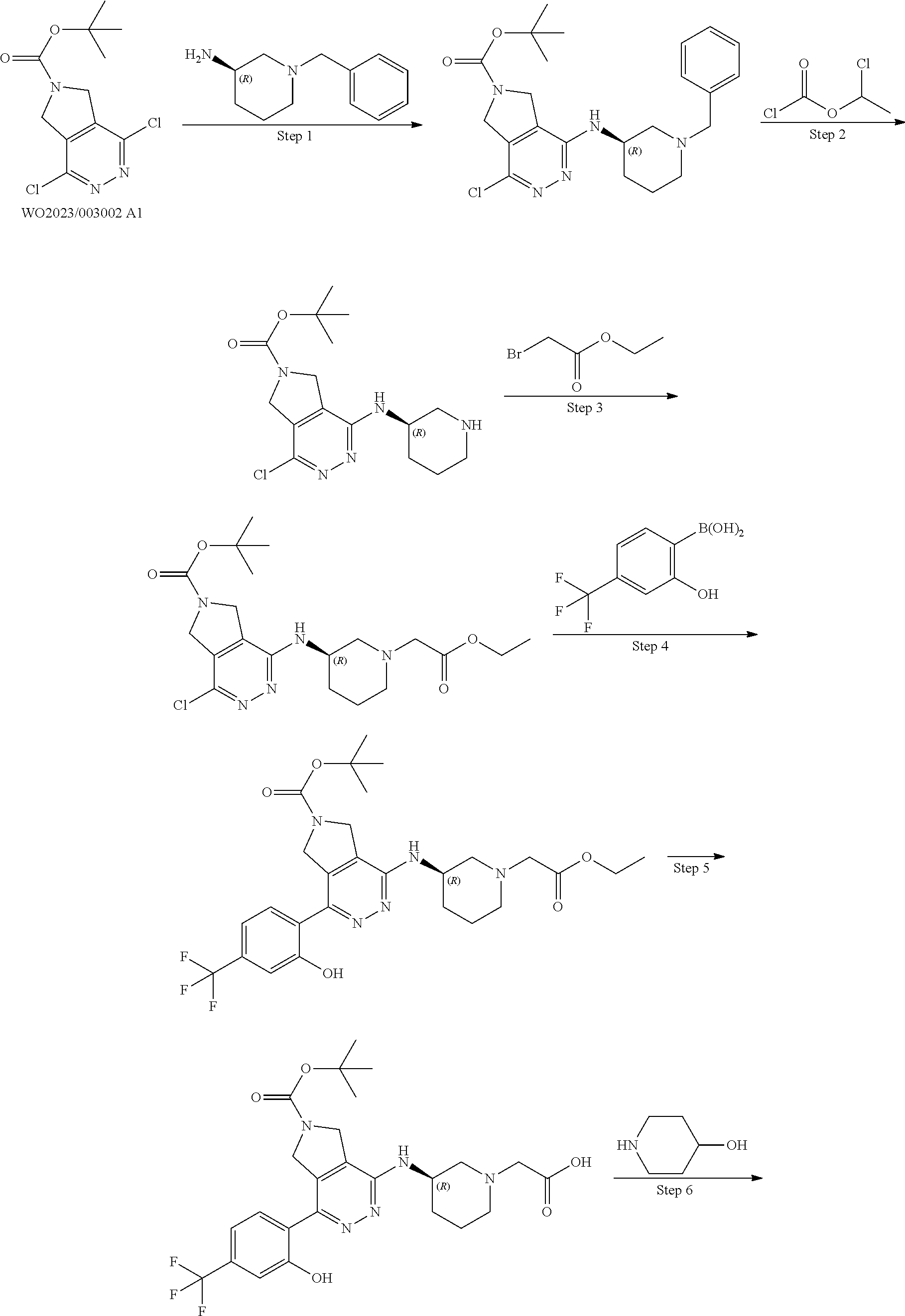

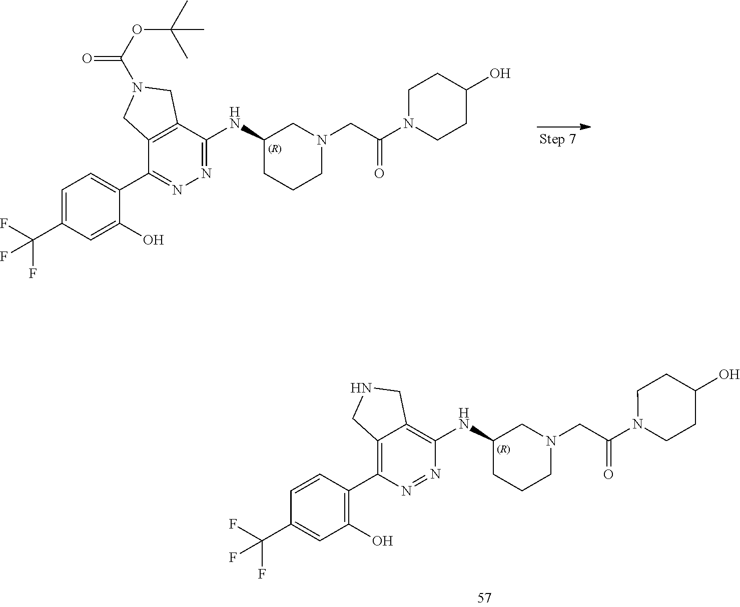

### Step 1: Synthesis of tert-butyl (R)-1-((1-benzylpiperidin-3-yl)amino)-4-chloro-5,7-dihydro-6H-pyrrolo[3,4-d]pyridazine-6-carboxylate

tert-Butyl 1,4-dichloro-5,7-dihydro-6H-pyrrolo[3,4-d]pyridazine-6-carboxylate (prepared as disclosed in international patent application publication WO2023/003002 A1) (1.0 g, 3.4 mmol, 1.0 eq.) and (R)-1-benzylpiperidin-3-amine (656 mg, 3.4 mmol, 1.0 eq.) were dissolved in 10 mL of NMP in a sealed tube, and DIPEA (1.20 mL, 891 mg, 6.9 mmol, 2.0 eq.) was added. The mixture was purged with nitrogen and then heated at 150° C. (heating block) for 16 hours in a sealed tube. Once LC/MS indicated the completion of the reaction, 50 mL of water was added into the reaction mixture. The aqueous layer was extracted with 5×30 mL of EtOAc. The organic extracts were combined, dried over sodium sulfate, filtered, and concentrated by rotary evaporation under reduced pressure. The residue was purified by flash column chromatography (0-2% MeOH in DCM) to afford tert-butyl (R)-1-((1-benzylpiperidin-3-yl)amino)-4-chloro-5,7-dihydro-6H-pyrrolo[3,4-d]pyridazine-6-carboxylate (1.0 g, 65%) as a yellow solid after evaporation of the combined product containing fractions under reduced pressure using a rotary evaporator. LC/MS: mass calcd for C₂₃H₃₀ClN₅O₂ 443.21, found m/z=444.05, [M+H]⁺. ¹H NMR (400 MHz, Chloroform-d): δ 7.36-7.24 (m, 5H), 5.10-4.86 (m, 1H), 4.66-4.47 (m, 4H), 4.39 (s, 1H), 3.64-3.49 (m, 1H), 3.49-3.31 (m, 1H), 2.82-2.53 (m, 2H), 2.50-2.34 (m, 1H), 2.33-2.12 (m, 1H), 1.95-1.81 (m, 1H), 1.57-1.49 (m, 11H) ppm.

### Step 2: Synthesis of tert-butyl (R)-1-chloro-4-(piperidin-3-ylamino)-5,7-dihydro-6H-pyrrolo[3,4-d]pyridazine-6-carboxylate

To a solution of tert-butyl (R)-1-((1-benzylpiperidin-3-yl)amino)-4-chloro-5,7-dihydro-6H-pyrrolo[3,4-d]pyridazine-6-carboxylate (1.0 g, 1.1 mmol, 1.0 eq.) in DCM (10 mL), 1-chloroethyl carbonochloridate (562 mg, 3.9 mmol, 3.5 eq.) was added at 0° C. (ice bath), and the reaction was allowed to stir with warming to rt for 1 hour. After the solution was concentrated by rotary evaporation under reduced pressure, the mixture was dissolved in 10 mL of methanol, and was refluxed at 70° C. (heating block) for 4 hours. After LC/MS indicated completion of the reaction, the solution was concentrated by rotary evaporation under reduced pressure. The crude material was purified by flash column chromatography (0-20% MeOH in DCM) to afford tert-butyl (R)-1-chloro-4-(piperidin-3-ylamino)-5,7-dihydro-6H-pyrrolo[3,4-d]pyridazine-6-carboxylate (640 mg, 80%) as a yellow solid after evaporation of the combined product containing fractions under reduced pressure using a rotary evaporator. LC/MS: mass calcd for C₁₆H₂₄ClN₅O₂ 353.16, found m/z=354.00, [M+H]⁺.

### Step 3: Synthesis of tert-butyl (R)-1-chloro-4-((1-(2-ethoxy-2-oxoethyl)piperidin-3-yl)amino)-5,7-dihydro-6H-pyrrolo[3,4-d]pyridazine-6-carboxylate

To a solution of tert-butyl (R)-1-chloro-4-(piperidin-3-ylamino)-5,7-dihydro-6H-pyrrolo[3,4-d]pyridazine-6-carboxylate (400 mg, 1.1 mmol, 1.0 eq.) in 20 mL of ACN, K₂CO₃ (312 mg, 2.2 mmol, 2.0 eq.) and ethyl 2-bromoacetate (170 mg, 1.0 mmol, 0.9 eq.) were added. The reaction was refluxed at 80° C. (heating block) for 4 hours. Once LC/MS indicated the completion of the reaction, the solution was passed through a Celite® cake, and the solution was concentrated by rotary evaporation under reduced pressure. The crude was purified by flash column chromatography (0-3% MeOH in DCM) to afford tert-butyl (R)-1-chloro-4-((1-(2-ethoxy-2-oxoethyl)piperidin-3-yl)amino)-5,7-dihydro-6H-pyrrolo[3,4-d]pyridazine-6-carboxylate (220 mg, 44%) as a yellow oil after evaporation of the combined product containing fractions under reduced pressure using a rotary evaporator. LC/MS: mass calcd for C₂₀H₃₀ClN₅O₄ 439.20, found m/z=440.05, [M+H]⁺. ¹H NMR (400 MHz, Chloroform-d): δ 4.69-4.53 (m, 4H), 4.44 (s, 2H), 4.18 (q, J=7.0 Hz, 2H), 3.28-3.16 (m, 1H), 2.88-2.32 (m, 3H), 2.04-1.89 (m, 1H), 1.85-1.70 (m, 1H), 1.63-1.52 (m, 3H), 1.52 (s, 9H), 1.27 (t, J=7.0 Hz, 3H) ppm.

### Step 4: Synthesis of tert-butyl (R)-1-((1-(2-ethoxy-2-oxoethyl)piperidin-3-yl)amino)-4-(2-hydroxy-4-(trifluoromethyl)phenyl)-5,7-dihydro-6H-pyrrolo[3,4-d]pyridazine-6-carboxylate

To a mixture of tert-butyl (R)-1-chloro-4-((1-(2-ethoxy-2-oxoethyl)piperidin-3-yl)amino)-5,7-dihydro-6H-pyrrolo[3,4-d]pyridazine-6-carboxylate (220 mg, 0.5 mmol, 1.0 eq.), 2-hydroxy-4-(trifluoromethyl)phenyl) boronic acid (206 mg, 1.0 mmol, 2.0 eq.), Pd(PPh₃)₄ (58 mg, 0.05 mmol, 0.1 eq.), and sodium carbonate (159 mg, 1.5 mmol, 3.0 eq.), 5 mL of 1,4-dioxane and 5 mL of water were added. The reaction was refluxed at 110° C. (heating block) for 16 hours under an atmosphere of nitrogen. The solution was cooled to room temperature, and 30 mL of water was added. The mixture solution was extracted with 3×30 mL of EA. The combined organic extracts were dried over sodium sulfate, filtered, and concentrated by rotary evaporation under reduced pressure. The residue was purified by flash column chromatography (0-35% ethyl acetate in petroleum ether) to afford tert-butyl (R)-1-((1-(2-ethoxy-2-oxoethyl)piperidin-3-yl)amino)-4-(2-hydroxy-4-(trifluoromethyl)phenyl)-5,7-dihydro-6H-pyrrolo[3,4-d]pyridazine-6-carboxylate (150 mg, 53%) as a yellow solid after evaporation of the combined product containing fractions under reduced pressure using a rotary evaporator. LC/MS: mass calcd for C₂₇H₃₄F₃N₅O₅ 565.25, found m/z=566.10, [M+H]⁺. ¹H NMR (400 MHz, Methanol-d₄): δ 7.53 (t, J=8.1 Hz, 1H), 7.25-7.15 (m, 2H), 4.64-4.53 (m, 4H), 4.43 (s, 2H), 4.18 (q, J=7.0 Hz, 2H), 3.06-2.90 (m, 1H), 2.71-2.42 (m, 3H), 1.95-1.75 (m, 2H), 1.73-1.59 (m, 3H), 1.52 (s, 9H), 1.27 (t, J=7.0 Hz, 3H) ppm. ¹⁹F NMR (376 MHz, Methanol-d₄): δ −64.53 ppm.

### Step 5: Synthesis of (R)-2-(3-((6-(tert-butoxycarbonyl)-4-(2-hydroxy-4-(trifluoromethyl)phenyl)-6,7-dihydro-5H-pyrrolo[3,4-d]pyridazin-1-yl)amino)piperidin-1-yl)acetic Acid

To a solution of tert-butyl (R)-1-((1-(2-ethoxy-2-oxoethyl)piperidin-3-yl)amino)-4-(2-hydroxy-4-(trifluoromethyl)phenyl)-5,7-dihydro-6H-pyrrolo[3,4-d]pyridazine-6-carboxylate (150 mg, 0.3 mmol, 1.0 eq.) in THF (2 mL) and MeOH (2 mL), LiOH·H₂O (50 mg, 1.2 mmol, 4.0 eq.) and 1 mL of water were added. The reaction was allowed to stir at rt for 1 hour, and the reaction progress was monitored by LC/MS. Organic solvents were removed by rotary evaporation under reduced pressure, and the residual aqueous solution was added 1M hydrochloric acid (HCl aq.) solution to adjust the pH value to 2-3 (pH paper). The white solid was filtered and dried under reduced pressure to afford (R)-2-(3-((6-(tert-butoxycarbonyl)-4-(2-hydroxy-4-(trifluoromethyl)phenyl)-6,7-dihydro-5H-pyrrolo[3,4-d]pyridazin-1-yl)amino)piperidin-1-yl)acetic acid (140 mg, 98%) as a yellow solid. LC/MS: mass calcd for C₂₅H₃₀F₃N₅O₅ 537.22, found m/z=538.10, [M+H]⁺.

### Step 6: Synthesis of tert-butyl (R)-1-(2-hydroxy-4-(trifluoromethyl)phenyl)-4-((1-(2-(4-hydroxypiperidin-1-yl)-2-oxoethyl)piperidin-3-yl)amino)-5,7-dihydro-6H-pyrrolo[3,4-d]pyridazine-6-carboxylate

To a solution of (R)-2-(3-((6-(tert-butoxycarbonyl)-4-(2-hydroxy-4-(trifluoromethyl)phenyl)-6,7-dihydro-5H-pyrrolo[3,4-d]pyridazin-1-yl)amino)piperidin-1-yl)acetic acid (100 mg, 0.2 mmol, 1.0 eq.) in 2 mL of DMF, DIPEA (65 μL, 48 mg, 0.4 mmol, 2.0 eq.) and HATU (141 mg, 0.4 mmol, 2.0 eq.) were added. The reaction was allowed to stir at rt for 1 hour, and then piperidin-4-ol (20 mg, 0.2 mmol, 1.0 eq.) was added. The reaction was allowed to stir at rt for 16 hours, and then most solvents were removed by rotary evaporation under reduced pressure. The crude material was purified by preparative TLC (10% MeOH in DCM) to afford (R)-2-(3-((6-(tert-butoxycarbonyl)-4-(2-hydroxy-4-(trifluoromethyl)phenyl)-6,7-dihydro-5H-pyrrolo[3,4-d]pyridazin-1-yl)amino)piperidin-1-yl)acetic acid (110 mg, 95%) as a yellow solid after eluting from the absorbent. LC/MS: mass calcd, for C₃₀H₃₉F₃N₆O₅ 620.29, found m/z=621.15, [M+H]⁺. ¹H NMR (400 MHz, Methanol-d₄): δ 7.52 (q, J=8.1 Hz, 1H), 7.24-7.15 (m, 2H), 4.63-4.49 (m, 2H), 4.45-4.36 (m, 1H), 4.05-3.76 (m, 2H), 3.70 (p, J=6.6 Hz, 2H), 3.56-3.35 (m, 2H), 3.17-2.94 (m, 2H), 2.85-2.71 (m, 1H), 2.47-2.34 (m, 1H), 2.05-1.77 (m, 4H), 1.77-1.57 (m, 2H), 1.47-1.37 (m, 2H), 1.34 (s, 9H) ppm. ¹⁹F NMR (376 MHz, Methanol-d₄): δ −64.54 ppm.

### Step 7: Synthesis of (R)-2-(3-((4-(2-hydroxy-4-(trifluoromethyl)phenyl)-6,7-dihydro-5H-pyrrolo[3,4-d]pyridazin-1-yl)amino)piperidin-1-yl)-1-(4-hydroxypiperidin-1-yl)ethan-1-one (57)

To a solution of (R)-2-(3-((6-(tert-butoxycarbonyl)-4-(2-hydroxy-4-(trifluoromethyl)phenyl)-6,7-dihydro-5H-pyrrolo[3,4-d]pyridazin-1-yl)amino)piperidin-1-yl)acetic acid (110 mg, 0.2 mmol, 1.0 eq.) in 2 mL of 1,4-dioxane, HCl (2 mL, 4 M in 1,4-dioxane, 0.8 mmol, 4.0 eq.) was added, and the solution was stirred at rt for 1 hour. The reaction solution was concentrated by rotary evaporation under reduced pressure, and the residue was purified by preparative RP-HPLC (5-65% ACN in water with 0.2% ammonium hydroxide). The collected pure fractions were freeze-dried to afford the target compound (R)-2-(3-((4-(2-hydroxy-4-(trifluoromethyl)phenyl)-6,7-dihydro-5H-pyrrolo[3,4-d]pyridazin-1-yl)amino)piperidin-1-yl)-1-(4-hydroxypiperidin-1-yl)ethan-1-one (57) (41 mg, 43%) as a pale yellow solid. LC/MS: mass calcd for C₂₅H₃₁F₃N₆O₃ 520.24, found m/z=521.10, [M+H]⁺. ¹H NMR (400 MHz, Methanol-d₄): δ 7.56 (t, J=8.0 Hz, 1H), 7.23-7.17 (m, 2H), 4.49-4.39 (m, 2H), 4.39-4.31 (m, 1H), 4.17-4.02 (m, 3H), 3.98-3.77 (m, 2H), 3.47-3.36 (m, 1H), 3.30-3.14 (m, 3H), 3.07-3.00 (m, 1H), 2.73-2.61 (m, 1H), 2.39-2.14 (m, 2H), 2.05-1.82 (m, 4H), 1.80-1.52 (m, 3H), 1.49-1.37 (m, 1H) ppm. ¹⁹F NMR (376 MHz, Methanol-d₄): δ −64.50 ppm.

### Example 58: (R)-2-(3-((6-(2-Hydroxy-4-(trifluoromethyl)phenyl)-5-methylpyridazin-3-yl)amino)piperidin-1-yl)-1-(4-(hydroxymethyl)piperidin-1-yl)ethan-1-one (58)

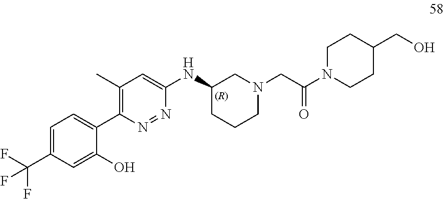

### Synthetic Route:

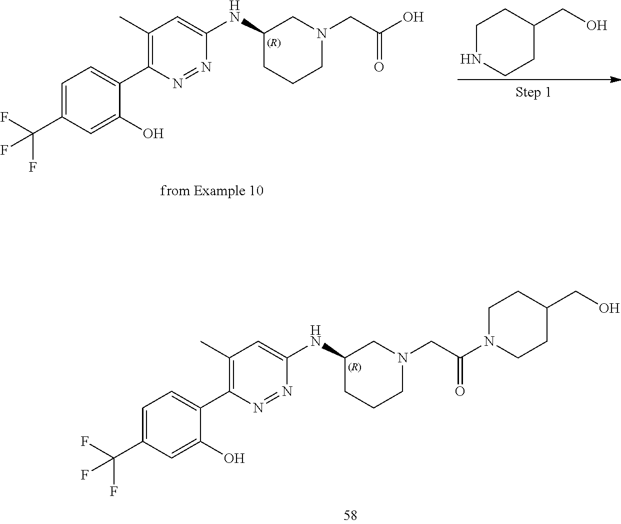

### Step 1: Synthesis of 1-(4-aminopiperidin-1-yl)-2-[(3R)-3-({6-[2-hydroxy-4-(trifluoromethyl)phenyl]-5-methylpyridazin-3-yl}amino)piperidin-1-yl]ethanone (58)

To a stirred solution of [(3R)-3-({6-[2-hydroxy-4-(trifluoromethyl)phenyl]-5-methylpyridazin-3-yl}amino)piperidin-1-yl]acetic acid (prepared as disclosed for Example 10) (166 mg, 0.4 mmol, 1.0 eq.) in pyridine (3 mL) was added piperidin-4-yl methanol (140 mg, 1.2 mmol, 3.0 eq.) and EDCI (116 mg, 0.6 mmol, 1.5 eq.) in portions at rt. The reaction mixture was stirred for 16 h at 50° C. (oil bath). The reaction mixture was concentrated under reduced pressure using a rotary evaporator. The residue was dissolved in DMF (1 mL) and purified by preparative RP-HPLC using the following conditions Column: YMC Triart C18 ExRs Column, 30*150 mm, 5 μm; Mobile Phase A: Water (10 mmol/L NH₄HCO₃), Mobile Phase B: ACN; Flow rate: 60 mL/min; Gradient: 21% B to 46% B in 10 min; Wavelengths: 254 nm/220 nm; RT1: 8.9 min to afford the target compound 2-[(3R)-3-({6-[2-hydroxy-4-(trifluoromethyl)phenyl]-5-methylpyridazin-3-yl}amino)piperidin-1-yl]-1-[4-(hydroxymethyl)piperidin-1-yl]ethanone (58) (35 mg, 16.3%) as a light yellow solid after lyophilization of the combined product containing fractions. LC/MS (ESI): mass calcd for C₂₅H₃₂F₃N₅O₃: 507.25. Found: m/z=508.15 [M+H]⁺. ¹H NMR (400 MHz, Methanol-d₄): δ 7.41 (d, J=8.0, 1H), 7.23 (d, J=8.0, 1H), 7.17 (s, 1H), 6.80 (s, 1H), 4.60-4.50 (m, 1H), 4.30-4.10 (m, 2H), 3.60-3.40 (m, 3H), 3.25-3.00 (m, 3H), 3.00-2.80 (m, 1H), 2.70-2.50 (m, 2H), 2.50-2.30 (m, 2H), 2.20-2.15 (m, 3H), 2.00-1.65 (m, 7H), 1.60-1.40 (m, 1H) ppm. ¹⁹F NMR (376 MHz, Methanol-d₄): δ −64.28 ppm.

### Example 59: 2-(4-(((R)-1-(((1r,3R)-3-Hydroxycyclobutyl)methyl)piperidin-3-yl)amino)-5,7-dihydrofuro[3,4-d]pyridazin-1-yl)-5-(trifluoromethyl)phenol (59)

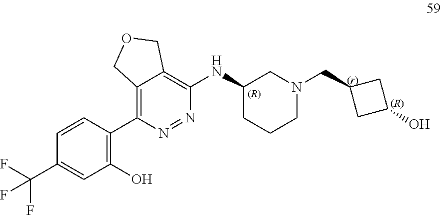

### Synthetic Route:

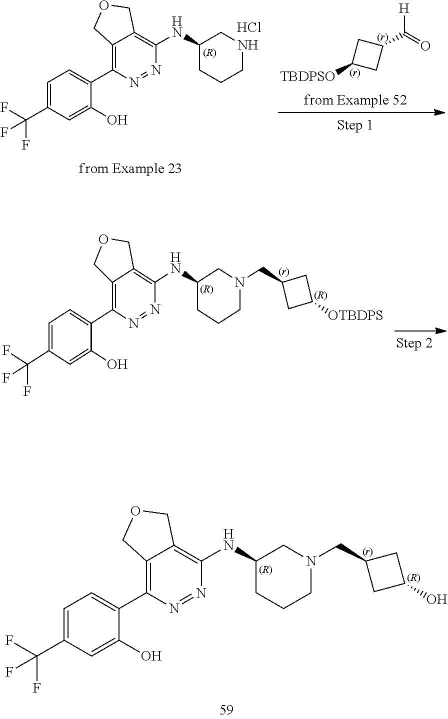

### Step 1: Synthesis of 2-(4-(((R)-1-(((1r,3R)-3-((tert-Butyldiphenylsilyl)oxy)cyclobutyl)methyl)piperidin-3-yl)amino)-5,7-dihydrofuro[3,4-d]pyridazin-1-yl)-5-(trifluoromethyl)phenol

In a single-necked flask, (1r,3r)-3-((tert-Butyldiphenylsilyl)oxy)cyclobutane-1-carbaldehyde (prepared as disclosed in Example 52) (1.4 g, 3.6 mmol, 12 eq.) and (R)-2-(4-(piperidin-3-ylamino)-5,7-dihydrofuro[3,4-d]pyridazin-1-yl)-5-(trifluoromethyl)phenol (prepared as disclosed in Example 23) (264 mg, 0.3 mmol, 1.0 eq.) were dissolved in 10 mL of MeOH followed by addition of acetic acid (1 mL, 17.5 mmol, 58.3 eq.) under an atmosphere of nitrogen. The mixture was stirred at rt for 30 min. Sodium triacetoxyborohydride (441 mg, 2.1 mmol, 7.0 eq.) was added in portions at 0° C. (ice bath), and the reaction mixture was stirred at rt overnight. Upon completion, the reaction mixture was quenched with 50 mL of water at 0° C. (ice bath) and extracted with 3×50 mL of EtOAc. The combined organic extracts were washed with 3×50 mL of saturated aqueous NaHCO₃ solution and dried over anhydrous sodium sulfate. After filtration, the filtrate was concentrated by rotary evaporation under reduced pressure. The residue was purified by flash column chromatography (2.5% MeOH in DCM) to afford 2-(4-(((R)-1-(((1r,3R)-3-((tert-butyldiphenylsilyl)oxy)cyclobutyl)methyl)piperidin-3-yl)amino)-5,7-dihydrofuro[3,4-d]pyridazin-1-yl)-5-(trifluoromethyl)phenol (355 mg, 73%) as a yellow solid after evaporation of the combined product containing fractions under reduced pressure using a rotary evaporator. LC/MS: mass calcd for C₃₉H₄₅F₃N₄O₃Si 702.32, found m/z=703.45 [M+H]⁺. ¹H NMR (400 MHz, Chloroform-d): δ 7.68-7.64 (m, 4H), 7.43-7.33 (m, 7H), 7.18-7.12 (m, 2H), 5.48 (t, J=3.1 Hz, 2H), 5.19 (s, 1H), 5.06-4.94 (m, 2H), 4.47 (s, 1H), 4.42-4.36 (m, 1H), 2.66 (d, J=9.4 Hz, 2H), 2.45-2.32 (m, 2H), 2.30-2.25 (m, 2H), 2.25-2.15 (m, 2H), 2.13-2.04 (m, 1H), 2.03-1.88 (m, 3H), 1.56 (s, 2H), 1.04 (s, 9H) ppm. ¹⁹F NMR (376 MHz, Chloroform-d): δ −63.18 ppm.

### Step 2: Synthesis of 2-(4-(((R)-1-(((1r,3R)-3-Hydroxycyclobutyl)methyl)piperidin-3-yl)amino)-5,7-dihydrofuro[3,4-d]pyridazin-1-yl)-5-(trifluoromethyl)phenol (59)

In a single-necked flask, 2-(4-(((R)-1-(((1r,3R)-3-((tert-butyldiphenylsilyl)oxy)cyclobutyl)methyl)piperidin-3-yl)amino)-5,7-dihydrofuro[3,4-d]pyridazin-1-yl)-5-(trifluoromethyl)phenol (350 mg, 0.5 mmol, 1.0 eq.) was dissolved in 10 mL of THF followed by the addition of tetrabutylammonium fluoride (314 mg, 1.0 mmol, 2.0 eq.) under an atmosphere of nitrogen. The reaction mixture was stirred at rt overnight. Upon completion, the reaction mixture was diluted with 50 mL of EtOAc and washed with 3×50 mL of saturated aqueous NaHCO₃ solution and dried over anhydrous sodium sulfate. After filtration, the filtrate was concentrated by rotary evaporation under reduced pressure. The residue was purified by flash column chromatography (6% MeOH in DCM) to afford the target compound 2-(4-(((R)-1-(((1r,3R)-3-hydroxycyclobutyl)methyl)piperidin-3-yl)amino)-5,7-dihydrofuro[3,4-d]pyridazin-1-yl)-5-(trifluoromethyl)phenol (59) (190 mg, 69%) as a yellow solid after evaporation of the combined product containing fractions under reduced pressure using a rotary evaporator. LC/MS: mass calcd, for C₂₃H₂₇F₃N₄O₃ 464.20, found m/z=465.30 [M+H]⁺. ¹H NMR (400 MHz, Methanol-d₄): δ 7.39 (d, J=8.9 Hz, 1H), 7.20-7.17 (m, 2H), 5.36 (t, J=3.1 Hz, 2H), 5.03 (t, J=3.1 Hz, 2H), 4.40-4.34 (m, 1H), 4.32-4.25 (m, 1H), 3.15-3.08 (m, 1H), 2.80-2.72 (m, 1H), 2.57-2.50 (m, 3H), 2.24-2.11 (m, 2H), 2.11-2.05 (m, 4H), 2.04-1.99 (m, 1H), 1.83-1.76 (m, 1H), 1.75-1.66 (m, 1H), 1.52-1.43 (m, 1H) ppm. ¹⁹F NMR (376 MHz, Methanol-d₄): δ −64.54 ppm.

### Example 60: (R)-2-(3-((4-(2-Hydroxy-4-(trifluoromethyl)phenyl)-6-methyl-6,7-dihydro-5H-pyrrolo[3,4-d]pyridazin-1-yl)amino)piperidin-1-yl)-1-(4-hydroxypiperidin-1-yl)ethan-1-one (60)

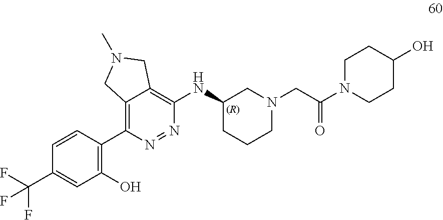

### Synthetic Route:

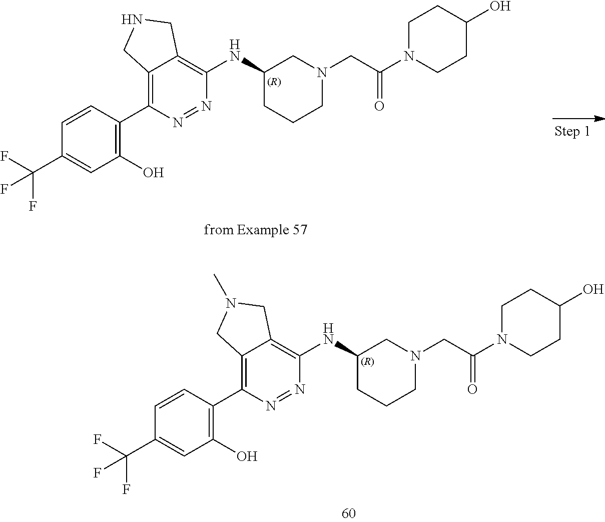

### Step 1: Synthesis of (R)-2-(3-((4-(2-hydroxy-4-(trifluoromethyl)phenyl)-6-methyl-6,7-dihydro-5H-pyrrolo[3,4-d]pyridazin-1-yl)amino)piperidin-1-yl)-1-(4-hydroxypiperidin-1-yl)ethan-1-one (60)

To a solution of (R)-2-(3-((4-(2-hydroxy-4-(trifluoromethyl)phenyl)-6,7-dihydro-5H-pyrrolo[3,4-d]pyridazin-1-yl)amino)piperidin-1-yl)-1-(4-hydroxypiperidin-1-yl)ethan-1-one (prepared as disclosed in Example 57) (42 mg, 0.08 mmol, 1.0 eq.) in 5 mL of MeOH, acetic acid (10 mg, 0.48 mmol, 6.0 eq.) and HCHO (6.5 mg, 37 wt. % in water, 0.08 mmol, 1.0 eq.) were added. The flask was allowed to stir at 50° C. for 1 hour. Then the reaction mixture was cooled to 0° C. (ice bath), and sodium cyanoborohydride (5 mg, 0.24 mmol, 3.0 eq.) was added. The mixture was then stirred at rt for an additional 2 hours. The reaction mixture was concentrated by rotary evaporation under reduced pressure, and the residue was purified by preparative RP-HPLC using 5-65% ACN in water (with 0.1 vol-% ammonium hydroxide) to afford the target compound (R)-2-(3-((4-(2-hydroxy-4-(trifluoromethyl)phenyl)-6-methyl-6,7-dihydro-5H-pyrrolo[3,4-d]pyridazin-1-yl)amino)piperidin-1-yl)-1-(4-hydroxypiperidin-1-yl)ethan-1-one (60) (13 mg, 30%) as a yellow solid after lyophilization of the combined product containing fractions. LC/MS: mass calcd for C₂₆H₃₃F₃N₆O₃ 534.26, found m/z=535.15, [M+H]⁺. ¹H NMR (400 MHz, Methanol-d₄): δ 7.52 (dd, J=7.7, 3.3 Hz, 1H), 7.23-7.17 (m, 2H), 4.39-4.30 (m, 1H), 4.24 (s, 1H), 4.18 (s, 1H), 4.15-4.04 (m, 1H), 3.96-3.79 (m, 4H), 3.49-3.33 (m, 1H), 3.29-3.09 (m, 3H), 3.07-3.00 (m, 1H), 2.64 (d, J=4.9 Hz, 4H), 2.37-2.15 (m, 2H), 2.06-1.91 (m, 2H), 1.89-1.80 (m, 2H), 1.77-1.66 (m, 1H), 1.62-1.33 (m, 3H) ppm. ¹⁹F NMR (376 MHz, Methanol-d₄): δ −64.49 ppm.

### Example 61: (R)-(3-((6-(2-Hydroxy-4-(trifluoromethyl)phenyl)-5-methylpyridazin-3-yl)amino)piperidin-1-yl) (6-hydroxyspiro[3.3]heptan-2-yl)methanone (61)

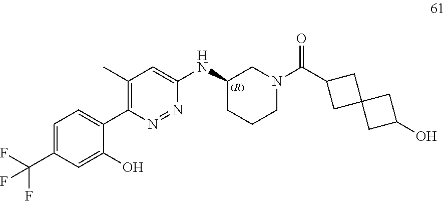

### Synthetic Scheme:

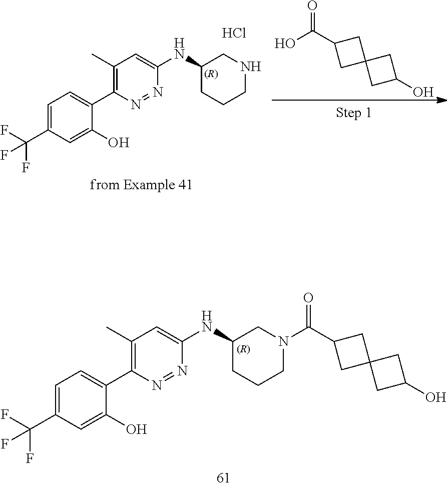

### Step 1: Synthesis of (R)-(3-((6-(2-Hydroxy-4-(trifluoromethyl)phenyl)-5-methylpyridazin-3-yl)amino)piperidin-1-yl) (6-hydroxyspiro[3.3]heptan-2-yl)methanone (61)

In a 50 mL round bottom flask, (R)-2-(4-methyl-6-(piperidin-3-ylamino)pyridazin-3-yl)-5-(trifluoromethyl)phenol hydrochloride (prepared as disclosed in Example 41) (124 mg, 0.35 mmol, 1.0 eq.), 6-hydroxyspiro[3.3]heptane-2-carboxylic acid (55 mg, 0.35 mmol, 1.0 eq.), EDAC (68 mg, 0.35 mmol, 1.0 eq.), HOBt (48 mg, 75%, containing water, 0.35 mmol, 1.0 eq.), DIPEA (0.18 mL, 3.0 eq.) were dissolved in 2 mL of DMF. The reaction mixture was stirred at rt overnight. Upon completion, the reaction mixture was purified by Biotage® reverse phase column chromatography (ACN/H₂O with 0.1% HCOOH). The collected fractions were combined to afford the target compound (R)-(3-((6-(2-Hydroxy-4-(trifluoromethyl)phenyl)-5-methylpyridazin-3-yl)amino)piperidin-1-yl) (6-hydroxyspiro[3.3]heptan-2-yl)methanone (61) as a white solid (66 mg, 38%) after lyophilization of the combined product containing fractions. LC/MS: mass calcd. for C₂₅H₂₉F₃N₄O₃ 490.2, found 490.5, m/z=[M+H]⁺. ¹H NMR (600 MHz, Methanol-d₄): δ 7.52-7.46 (m, 1H), 7.38 (s, 1H), 7.33-7.29 (m, 1H), 7.27-7.20 (m, 1H), 4.24-4.11 (m, 1H), 4.10-4.01 (m, 1H), 3.98-3.81 (m, 2H), 3.67-3.53 (m, 1H), 3.26-3.21 (m, 1H), 2.58-2.45 (m, 1H), 2.44-2.31 (m, 1H), 2.30-2.27 (m, 3H), 2.26-2.13 (m, 4H), 2.03-1.93 (m, 1H), 1.93-1.77 (m, 3H), 1.77-1.69 (m, 1H), 1.66-1.55 (m, 1H), 1.45-1.25 (m, 1H) ppm. ¹⁹F NMR (564 MHz, Methanol-d₄): δ −64.60 ppm.

### Example 62: (R)-6-((3-((6-(2-Hydroxy-4-(trifluoromethyl)phenyl)-5-methylpyridazin-3-yl)amino)piperidin-1-yl)methyl)spiro[3.3]heptan-2-ol (62)

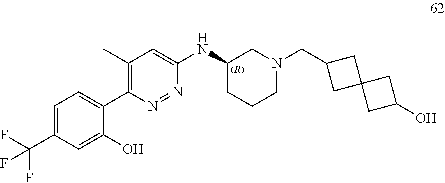

### Synthetic Route:

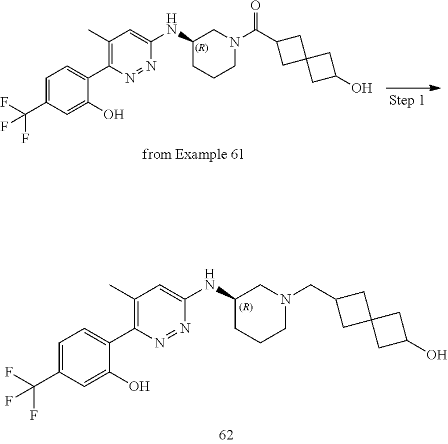

### Step 1: Synthesis of (R)-6-((3-((6-(2-hydroxy-4-(trifluoromethyl)phenyl)-5-methylpyridazin-3-yl)amino)piperidin-1-yl)methyl)spiro[3.3]heptan-2-ol (62)

In a 50 mL round bottom flask, (R)-(3-((6-(2-hydroxy-4-(trifluoromethyl)phenyl)-5-methylpyridazin-3-yl)amino)piperidin-1-yl) (6-hydroxyspiro[3.3]heptan-2-yl)methanone (prepared as disclosed in Example 61) (20 mg, 0.041 mmol, 1.0 eq.) was added. The flask was sealed with a stopper and connected with a Schlenk line to undergo nitrogen/vacuum cycling 3 times. 4 mL of anhydrous THF was added and the mixture was stirred at 0° C. (ice bath) under an atmosphere of nitrogen. LiAlH₄ (1 M in THE, 0.20 mL, 0.20 mmol, 5.0 eq.) was added dropwise at 0° C. (ice bath). The mixture was then heated to 60° C. (heating block) and stirred for 3 h. Upon completion, the mixture was cooled to 0° C. (ice bath), quenched with 10 mL of saturated aqueous NH₄Cl solution, and extracted with EtOAc 10 mL×3. The combined organic extracts were dried over anhydrous Na₂SO₄. After filtration, the filtrate was concentrated by rotary evaporation under reduced pressure. The crude material was further purified by preparative RP-HPLC (ACN/H₂O with 0.1% HCOOH). The combined fractions were freeze-dried to afford the target compound (R)-6-((3-((6-(2-hydroxy-4-(trifluoromethyl)phenyl)-5-methylpyridazin-3-yl)amino)piperidin-1-yl)methyl)spiro[3.3]heptan-2-ol (62) as a pale yellow solid (9 mg, 46%). LC/MS: mass calcd for C₂₅H₃₁F₃N₄O₂ 476.2, found 476.7, m/z=[M+H]⁺. ¹H NMR (600 MHz, Methanol-d₄): δ 7.39 (d, J=7.4 Hz, 1H), 7.25 (dd, J=7.5, 1.4 Hz, 1H), 7.19 (d, J=1.5 Hz, 1H), 6.84 (s, 1H), 4.35-4.27 (m, 1H), 4.10-4.03 (m, 1H), 3.75-3.56 (m, 1H), 3.09 (d, J=7.2 Hz, 2H), 3.03-2.77 (m, 2H), 2.66-2.59 (m, 1H), 2.51-2.44 (m, 1H), 2.27-2.20 (m, 2H), 2.19-2.16 (m, 1H), 2.14 (d, J=1.0 Hz, 3H), 2.13-2.07 (m, 2H), 1.96-1.78 (m, 6H), 1.74-1.62 (m, 1H) ppm. ¹⁹F NMR (564 MHz, Methanol-d₄): δ −64.30 ppm.

### Example 63: (R)-(3-((6-(2-Hydroxy-4-(trifluoromethyl)phenyl)-5-methylpyridazin-3-yl)amino)piperidin-1-yl) (3-hydroxybicyclo[1.1.1]pentan-1-yl)methanone (63)

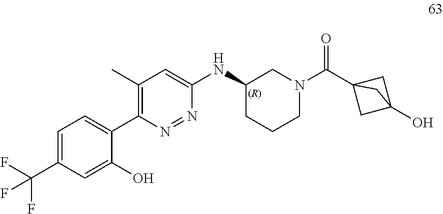

### Synthetic Route:

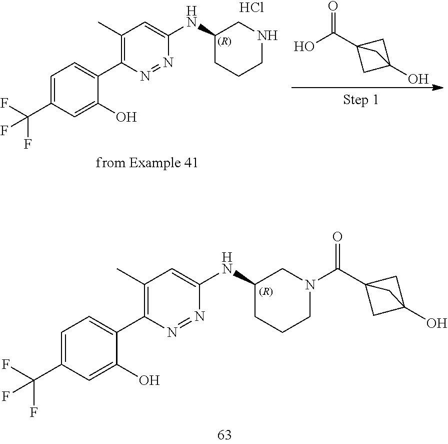

### Step 1: Synthesis of (R)-(3-((6-(2-hydroxy-4-(trifluoromethyl)phenyl)-5-methylpyridazin-3-yl)amino)piperidin-1-yl) (3-hydroxybicyclo[1.1.1]pentan-1-yl)methanone (63)

In a 50 mL round bottom flask, (R)-2-(4-methyl-6-(piperidin-3-ylamino)pyridazin-3-yl)-5-(trifluoromethyl)phenol hydrochloride (prepared as disclosed in Example 41) (124 mg, 0.35 mmol, 1.0 eq.), 3-hydroxybicyclo[1.1.1]pentane-1-carboxylic acid (45 mg, 0.35 mmol, 1.0 eq.), EDAC (68 mg, 0.35 mmol, 1.0 eq.), HOBt (48 mg, 75%, containing water, 0.35 mmol, 1.0 eq.), DIPEA (0.18 mL, 3 eq.) were dissolved in 2 mL of anhydrous DMF. The mixture was stirred at rt overnight. Upon completion, the crude material was purified by Biotage® reverse phase column chromatography (ACN/H₂O with 0.1% HCOOH). The collected fractions were combined and freeze-dried to afford the target compound (R)-(3-((6-(2-hydroxy-4-(trifluoromethyl)phenyl)-5-methylpyridazin-3-yl)amino)piperidin-1-yl) (3-hydroxybicyclo[1.1.1]pentan-1-yl)methanone (63) as a pale yellow solid (103 mg, 63%). LC/MS: mass calcd for C₂₃H₂₅F₃N₄O₃ 462.2, found 462.8, m/z=[M+H]⁺. ¹H NMR (600 MHz, Methanol-d₄): δ 7.46-7.37 (m, 1H), 7.29-7.21 (m, 1H), 7.21-7.11 (m, 1H), 6.86-6.78 (m, 1H), 4.35-4.21 (m, 1H), 4.05-3.85 (m, 2H), 3.29-3.22 (m, 1H), 3.22-3.10 (m, 1H), 2.32-2.26 (m, 2H), 2.23 (s, 2H), 2.19-2.02 (m, 6H), 1.91-1.83 (m, 1H), 1.74-1.55 (m, 2H) ppm. ¹⁹F NMR (564 MHz, Methanol-d₄): δ −64.30 ppm.

### Example 64: (R)-3-((3-((6-(2-Hydroxy-4-(trifluoromethyl)phenyl)-5-methylpyridazin-3-yl)amino)piperidin-1-yl)methyl) bicyclo[1.1.1]pentan-1-ol (64)

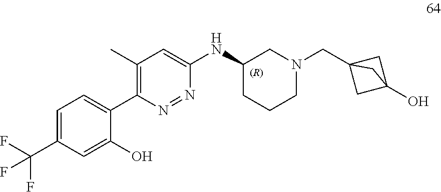

### Synthetic Route:

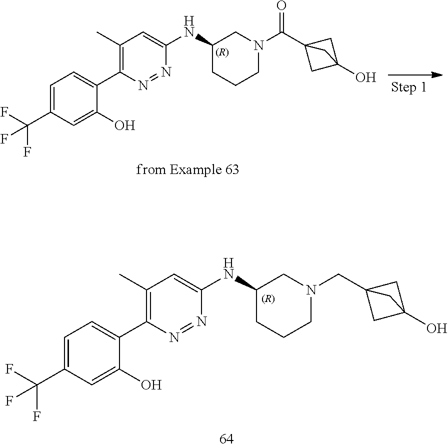

### Step 1: Synthesis of (R)-3-((3-((6-(2-hydroxy-4-(trifluoromethyl)phenyl)-5-methylpyridazin-3-yl)amino)piperidin-1-yl)methyl) bicyclo[1.1.1]pentan-1-ol (64)

In a 50 mL round bottom flask, (R)-(3-((6-(2-hydroxy-4-(trifluoromethyl)phenyl)-5-methylpyridazin-3-yl)amino)piperidin-1-yl) (3-hydroxybicyclo[1.1.1]pentan-1-yl)methanone (prepared as disclosed in Example 63) (60 mg, 0.13 mmol, 1.0 eq.) was added. The flask was sealed with a rubber septum and connected toa Schlenk line to undergo N₂/vacuum cycling 3 times. 4 mL of anhydrous THF was added and the mixture was stirred at 0° C. (ice bath) under an atmosphere of nitrogen. LiAlH₄ (1 M in THF, 0.65 mL, 0.65 mmol, 5.0 eq.) was added dropwise at 0° C. (ice bath). The mixture was heated to 60° C. (heating block) and stirred for 2 h. Upon completion, the mixture was cooled to 0° C. (ice bath), quenched with 10 mL of a saturated aqueous NH₄Cl solution and extracted with EtOAc 10 mL×3. The combined organic extracts were dried over anhydrous Na₂SO₄. After filtration, the filtrate was concentrated by rotary evaporation under reduced pressure. The crude was further purified by preparative RP-HPLC (5-65% ACN/H₂O with 0.1% HCOOH). The collected fractions were freeze-dried to afford the target compound (R)-3-((3-((6-(2-hydroxy-4-(trifluoromethyl)phenyl)-5-methylpyridazin-3-yl)amino)piperidin-1-yl)methyl) bicyclo[1.1.1]pentan-1-ol (64) (25 mg, 43%) as yellow solid. LC/MS: mass calcd for C₂₃H₂₇F₃N₄O₂ 448.2, found 448.7, m/z=[M+H]⁺. ¹H NMR (600 MHz, Methanol-d₄): δ 7.40 (d, J=7.8 Hz, 1H), 7.23 (d, J=7.9 Hz, 1H), 7.18 (s, 1H), 6.80 (s, 1H), 4.25-4.16 (m, 1H), 3.49-3.36 (m, 1H), 3.14-3.02 (m, 1H), 2.97 (s, 2H), 2.69-2.46 (m, 2H), 2.14 (s, 3H), 2.09-2.02 (m, 1H), 2.00-1.94 (m, 1H), 1.93-1.86 (m, 6H), 1.86-1.76 (m, 1H), 1.61-1.50 (m, 1H) ppm. ¹⁹F NMR (564 MHz, Methanol-d₄): δ −64.29 ppm.

### Example 65: (R)-1-(3-((6-(2-hydroxy-4-(trifluoromethyl)phenyl)-5-methylpyridazin-3-yl)amino)piperidin-1-yl)-2-(4-hydroxycyclohexyl)ethan-1-one (65)—Fraction A and Fraction B

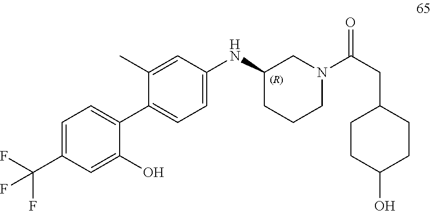

### Synthetic Route:

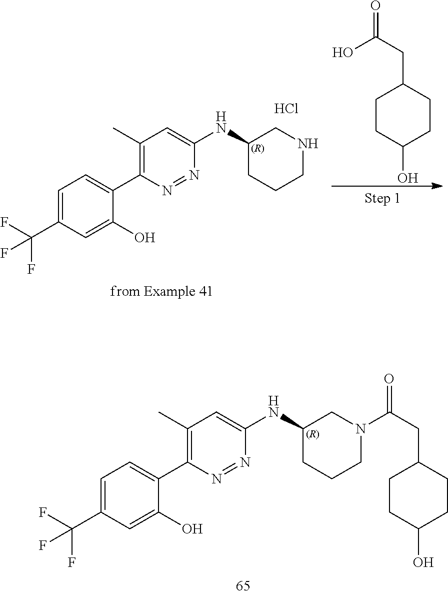

### Step 1: Synthesis of (R)-1-(3-((6-(2-hydroxy-4-(trifluoromethyl)phenyl)-5-methylpyridazin-3-yl)amino)piperidin-1-yl)-2-(4-hydroxycyclohexyl)ethan-1-one (65)

In a single-necked flask, 2-(4-hydroxycyclohexyl)acetic acid (45 mg, 0.3 mmol, 1.0 eq.) was dissolved in 2 mL of DMF followed by DIPEA (100 μL, 74 mg, 0.6 mmol, 2.0 eq.) and HATU (130 mg, 0.3 mmol, 1.2 eq.) under an atmosphere of nitrogen. The reaction mixture was stirred at rt for 1 h. (R)-2-(4-methyl-6-(piperidin-3-ylamino)pyridazin-3-yl)-5-(trifluoromethyl)phenol (prepared as disclosed in Example 41) (100 mg, 0.3 mmol, 1.0 eq.) was added and the reaction mixture was stirred at rt for 2 h. Upon completion, the reaction mixture was concentrated by rotary evaporation under reduced pressure. The residue was purified by preparative RP HPLC (5-65% ACN in H₂O, 0.1% HCOOH) to afford the target compound (R)-1-(3-((6-(2-hydroxy-4-(trifluoromethyl)phenyl)-5-methylpyridazin-3-yl)amino)piperidin-1-yl)-2-(4-hydroxycyclohexyl)ethan-1-one (65) as two fractions Fraction A (18 mg, 29%) and Fraction B (11 mg, 18%) both as a white solids after lyophilization of the combined product containing fractions. The compounds reflect cis/trans isomerism and an absolute and relative configuration was not determined.

### Fraction A:

LC/MS: mass calcd for C₂₅H₃₁F₃N₄O₃ 492.23, found m/z=493.35 [M+H]⁺. ¹H NMR (400 MHz, Methanol-d₄): δ (d, J=8.4 Hz, 1H), 7.21 (d, J=7.8 Hz, 1H), 7.15 (d, J=5.0 Hz, 1H), 6.79 (d, J=5.0 Hz, 1H), 4.07-3.93 (m, 1H), 3.92-3.81 (m, 1H), 3.77-3.61 (m, 1H), 3.58-3.31 (m, 3H), 2.33-2.24 (m, 1H), 2.18-2.14 (m, 1H), 2.12 (s, 3H), 2.10-1.96 (m, 1H), 1.95-1.52 (m, 8H), 1.30-0.83 (m, 4H) ppm. ¹⁹F NMR (376 MHz, Methanol-d₄): δ −64.33 ppm.

### Fraction B:

LC/MS: mass calcd, for C₂₅H₃₁F₃N₄O₃ 492.23, found m/z=493.35 [M+H]⁺. ¹H NMR (400 MHz, Methanol-d₄): δ 7.40 (d, J=7.9 Hz, 1H), 7.23 (d, J=7.9 Hz, 1H), 7.16 (s, 1H), 6.81 (s, 1H), 4.07-3.86 (m, 2H), 3.85-3.71 (m, 2H), 3.52-3.38 (m, 1H), 2.37-2.28 (m, 1H), 2.26-2.16 (m, 1H), 2.14 (s, 3H), 2.12-1.91 (m, 1H), 1.90-1.29 (m, 13H) ppm. ¹⁹F NMR (376 MHz, Methanol-d₄): δ −64.32 ppm.

### Example 66: (R)-2-(6-((1-(2-(3-Hydroxycyclobutyl)ethyl)piperidin-3-yl)amino)-4-methylpyridazin-3-yl)-5-(trifluoromethyl)phenol (66)

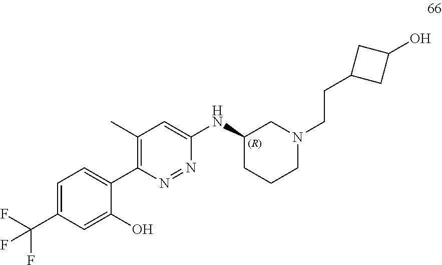

### Synthetic Route:

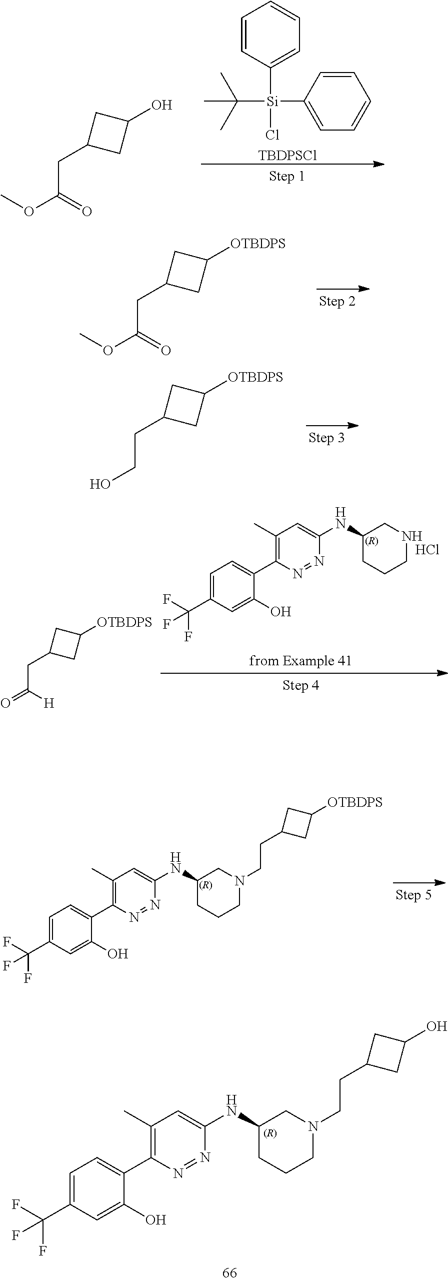

### Step 1: Synthesis of methyl 2-(3-((tert-butyldiphenylsilyl)oxy)cyclobutyl)acetate

To a solution of methyl 2-(3-hydroxycyclobutyl)acetate (200 mg, 1.4 mmol, 1.0 eq.) in 4 mL of DCM, tert-butyldiphenylchlorosilane (571 mg, 2.1 mmol, 1.5 eq.) and 1H-imidazole (207 mg, 3.11 mmol, 2.2 eq.) were added at 0° C. (ice bath). The mixture was stirred at rt overnight. The reaction solution was then filtered through a Celite® cake and washed with 2×10 mL of DCM. The combined filtrate was concentrated by rotary evaporation under reduced pressure, and the residue was purified by flash column chromatography (4% EtOAc in PE) to afford methyl 2-(3-((tert-butyldiphenylsilyl)oxy)cyclobutyl)acetate (490 mg, 92%) as a colorless oil after evaporation of the product containing fractions under reduced pressure using a rotary evaporator. LC/MS: mass calcd for C₂₃H₃₀O₃Si 382.20, found m/z=383.10, [M+H]⁺. ¹H NMR (400 MHz, Chloroform-d): δ 7.65-7.60 (m, 4H), 7.42-7.32 (m, 6H), 4.06 (p, J=7.4 Hz, 1H), 3.60 (s, 3H), 2.40 (d, J=7.4 Hz, 2H), 2.36-2.26 (m, 2H), 1.98-1.88 (m, 1H), 1.78-1.66 (m, 2H), 1.00 (s, 9H) ppm.

### Step 2: Synthesis of 2-(3-((tert-butyldiphenylsilyl)oxy)cyclobutyl)ethan-1-ol

To a solution of methyl 2-(3-((tert-butyldiphenylsilyl)oxy)cyclobutyl)acetate (490 mg, 1.3 mmol, 1.0 eq.) in 15 mL of THE, a LiBH₄ solution (1N in THF, 2.6 mL, 2.6 mmol, 2.0 eq.) was added at 0° C. (ice bath), and then the mixture is stirred with gradual warming to rt overnight. To quench the reaction, the mixture was cooled to 0° C. (ice bath), and 2 mL of a saturated aqueous NH₄Cl solution was added, followed by addition of 10 mL of EtOAc. The mixture was filtered through a Celite® cake, and the cake was further washed with 10 mL of EtOAc. The combined filtrates were washed with 3×20 mL of brine, dried over sodium sulfate, filtered, and concentrated by rotary evaporation under reduced pressure. The residue was purified by flash column chromatography (6% EtOAc in PE) to afford 2-(3-((tert-butyldiphenylsilyl)oxy)cyclobutyl)ethan-1-ol (450 mg, 99%) as a colorless oil after evaporation of the product containing fractions under reduced pressure using a rotary evaporator. LC/MS: mass calcd, for C₂₂H₃₀O₂Si 354.20, found m/z=355.10, [M+H]⁺. ¹H NMR (400 MHz, Methanol-d₄): δ 7.65-7.59 (m, 4H), 7.43-7.33 (m, 6H), 4.07 (p, J=7.3 Hz, 1H), 3.47-3.38 (m, 2H), 2.32-2.17 (m, 2H), 1.68-1.53 (m, 5H), 0.99 (s, 9H) ppm.

### Step 3: Synthesis of 2-(3-((tert-butyldiphenylsilyl)oxy)cyclobutyl)acetaldehyde

To a solution of 2-(3-((tert-butyldiphenylsilyl)oxy)cyclobutyl)ethan-1-ol (450 mg, 1.2 mmol, 1.0 eq.) in 10 mL of DCM, PCC (507 mg, 2.4 mmol, 2.0 eq.) was added at 0° C. (ice bath), and the mixture was stirred with gradual warming to rt overnight. The reaction mixture was purified without any evaporation by flash column chromatography (100% DCM) (isocratic) to afford 2-(3-((tert-butyldiphenylsilyl)oxy)cyclobutyl)acetaldehyde (188 mg, 42%) as a colorless oil after evaporation of the product containing fractions under reduced pressure using a rotary evaporator. ¹H NMR (400 MHz, Chloroform-d): δ 9.68 (s, 1H), 7.64 (d, J=6.9 Hz, 4H), 7.42-7.35 (m, 6H), 4.11 (p, J=7.4 Hz, 1H), 2.55 (d, J=7.1 Hz, 2H), 2.44-2.34 (m, 2H), 2.03-1.92 (m, 1H), 1.81-1.68 (m, 2H), 1.02 (s, 9H) ppm.

### Step 4: Synthesis of (R)-2-(6-((1-(2-(3-((tert-butyldiphenylsilyl)oxy)cyclobutyl)ethyl)piperidin-3-yl)amino)-4-methylpyridazin-3-yl)-5-(trifluoromethyl)phenol

In a single-necked flask, 2-(3-((tert-butyldiphenylsilyl)oxy)cyclobutyl)acetaldehyde (150 mg, 0.4 mmol, 1.5 eq.) and (R)-2-(4-methyl-6-(piperidin-3-ylamino)pyridazin-3-yl)-5-(trifluoromethyl)phenol (prepared as disclosed in Example 41) (100 mg, 0.4 mmol, 1.0 eq.) were dissolved in 3 mL of MeOH, and then 3 drops of acetic acid were added. After stirring at rt for 30 minutes, the mixture was cooled to 0° C. (ice bath), and NaBH₃CN (54 mg, 0.9 mmol, 2.0 eq.) was added. The mixture was stirred at rt overnight. The reaction was quenched by dropwise addition of water at 0° C. (ice bath). The mixture was extracted with 3×20 mL EtOAc, and the combined organic extracts were washed with 3×20 mL of a saturated aqueous NaHCO₃ solution, dried over sodium sulfate, filtered, and concentrated by rotary evaporation under reduced pressure. The crude product was purified by flash column chromatography (4% DCM in MeOH) to afford (R)-2-(6-((1-(2-(3-((tert-butyldiphenylsilyl)oxy)cyclobutyl)ethyl)piperidin-3-yl)amino)-4-methylpyridazin-3-yl)-5-(trifluoromethyl)phenol (140 mg, 71%) as a yellow solid after evaporation of the product containing fractions under reduced pressure using a rotary evaporator. LC/MS: mass calcd, for C₃₉H₄₇F₃N₄O₂Si 688.34, found m/z=689.40, [M+H]⁺. ¹H NMR (400 MHz, Methanol-d₄): δ 7.66-7.61 (m, 4H), 7.46-7.29 (m, 8H), 7.24 (d, J=8.1 Hz, 1H), 7.18 (s, 1H), 6.81 (s, 1H), 4.33-4.22 (m, 1H), 4.17-4.06 (m, 1H), 3.49-3.34 (m, 1H), 3.31 (s, 1H), 3.04-2.84 (m, 3H), 2.39-2.27 (m, 2H), 2.21-2.04 (m, 5H), 2.02-1.52 (m, 8H), 1.00 (s, 9H) ppm. ¹⁹F NMR (376 MHz, Methanol-d₄): δ −63.34 ppm.

### Step 5: Synthesis of (R)-2-(6-((1-(2-(3-hydroxycyclobutyl)ethyl)piperidin-3-yl)amino)-4-methylpyridazin-3-yl)-5-(trifluoromethyl)phenol (66)

To a solution of (R)-2-(6-((1-(2-(3-((tert-butyldiphenylsilyl)oxy)cyclobutyl)ethyl)piperidin-3-yl)amino)-4-methylpyridazin-3-yl)-5-(trifluoromethyl)phenol (140 mg, 0.4 mmol, 1.0 eq.) in 50 mL of THF, TBAF (207 mg, 0.8 mmol, 2.0 eq.) was added, and the mixture was stirred at rt overnight. The reaction was concentrated by rotary evaporation under reduced pressure, and then 30 mL of a saturated aqueous NaHCO₃ solution was added. The mixture was extracted with 2×20 mL of EtOAc. The combined organic extracts were dried over sodium sulfate, filtered, and concentrated by rotary evaporation under reduced pressure. The residue was purified by flash column chromatography (6% MeOH in DCM) to afford the target compound (R)-2-(6-((1-(2-(3-hydroxycyclobutyl)ethyl)piperidin-3-yl)amino)-4-methylpyridazin-3-yl)-5-(trifluoromethyl)phenol (66) (37 mg, 40%) as a pale yellow solid after evaporation of the product containing fractions under reduced pressure using a rotary evaporator.

LC/MS: mass calcd for C₂₃H₂₉F₃N₄O₂ 450.22, found m/z=451.15, [M+H]⁺. ¹H NMR (400 MHz, Methanol-d₄): δ 7.37 (d, J=8.6 Hz, 1H), 7.20 (d, J=7.9 Hz, 1H), 7.14 (s, 1H), 6.75 (s, 1H), 4.18-4.07 (m, 1H), 3.99 (p, J=7.6 Hz, 1H), 3.21-3.11 (m, 1H), 2.86-2.75 (m, 1H), 2.45-2.31 (m, 4H), 2.28-2.19 (m, 1H), 2.15 (brs, 4H), 2.06-1.95 (m, 2H), 1.87-1.78 (m, 1H), 1.70-1.60 (m, 4H), 1.52-1.44 (m, 2H) ppm. ¹⁹F NMR (376 MHz, Methanol-d₄): δ −63.34 ppm.

### Example 67: (R)-(3-((6-(2-Hydroxy-4-(trifluoromethyl)phenyl)-4,5-dimethylpyridazin-3-yl)amino)piperidin-1-yl) (3-hydroxybicyclo[1.1.1]pentan-1-yl)methanone (67)

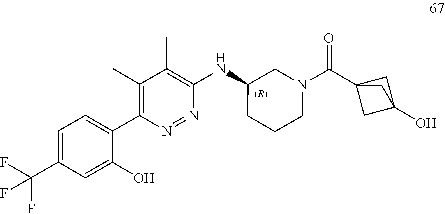

### Synthetic Route:

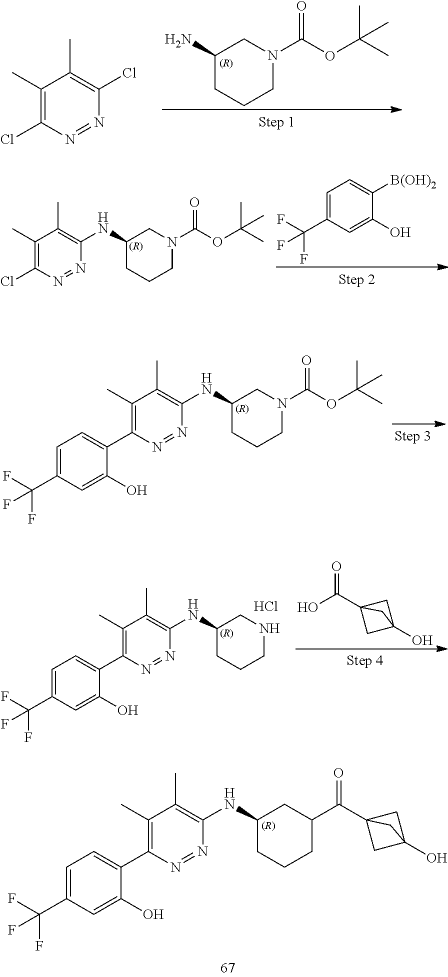

### Step 1: Synthesis of tert-butyl (R)-3-((6-chloro-4,5-dimethylpyridazin-3-yl)amino)piperidine-1-carboxylate

To a 250 mL sealed tube were added 3,6-dichloro-4,5-dimethylpyridazine (15.9 g, 90 mmol, 1.0 eq.), tert-butyl (R)-3-aminopiperidine-1-carboxylate (18.0 g, 90 mmol, 1.0 eq.), and NMP (50 mL) followed by addition of DIPEA (48 mL, 35.6 g, 270 mmol, 3.0 eq.). The mixture was heated at 150° C. and stirred overnight. Upon completion, the mixture was allowed to cool to room temperature. The mixture was diluted with EtOAc (300 mL) and washed with water (100 mL×3), 1 M hydrochloric acid (HCl aq.) solution (100 mL), and brine (100 mL×3). The combined organic extracts were dried over sodium sulfate. After filtration, the filtrate was concentrated by rotary evaporation under reduced pressure. The residue was purified by flash column chromatography (DCM:EtOAc=5:1) to afford the tert-butyl (R)-3-((6-chloro-4,5-dimethylpyridazin-3-yl)amino)piperidine-1-carboxylate (1.97 g, 6%) as an off-white solid after evaporation of the combined product containing fractions under reduced pressure using a rotary evaporator. LC/MS: mass calcd, for C₁₆H₂₅ClN₄O₂: 340.2, found 340.8, m/z=[M+H]⁺. ¹H NMR (600 MHz, Chloroform-d): δ 4.42-4.22 (m, 1H), 3.90-3.56 (m, 2H), 3.55-3.29 (m, 1H), 3.27-3.00 (m, 1H), 2.30 (s, 3H), 2.06 (s, 3H), 1.97-1.75 (m, 2H), 1.74-1.63 (m, 1H), 1.62-1.51 (m, 1H), 1.50-1.30 (m, 9H) ppm.

### Step 2: Synthesis of tert-butyl (R)-3-((6-(2-hydroxy-4-(trifluoromethyl)phenyl)-4,5-dimethylpyridazin-3-yl)amino)piperidine-1-carboxylate

To a 250 mL round bottom flask were added tert-butyl (R)-3-((6-chloro-4,5-dimethylpyridazin-3-yl)amino)piperidine-1-carboxylate (1.9 g, 5.6 mmol, 1.0 eq.), (2-hydroxy-4-(trifluoromethyl)phenyl) boronic acid (1.7 g, 8.4 mmol, 1.5 eq.), and Na₂CO₃ (1.8 g, 16.8 mmol, 3.0 eq.), Pd(PPh₃)₄ (693 mg, 0.6 mmol, 0.1 eq.). The mixture was degassed under vacuum and refilled with nitrogen three times. 30 mL of 1,4-dioxane and 8.4 mL of water were added, and the mixture was stirred at 100° C. overnight. Upon completion, the mixture was filtered through a Celite® cake and the filtrate was concentrated by rotary evaporation under reduced pressure. The residue was purified by flash column chromatography (30% EtOAc in hexane) to afford tert-butyl (R)-3-((6-(2-hydroxy-4-(trifluoromethyl)phenyl)-4,5-dimethylpyridazin-3-yl)amino)piperidine-1-carboxylate as an off-white solid (1.6 g, 61%) after evaporation of the combined product containing fractions under reduced pressure using a rotary evaporator. MS: mass calcd for C₂₃H₂₉F₃N₄O₃ 466.2, found 466.6, m/z=[M+H]⁺. ¹H NMR (600 MHz, Methanol-d₄): δ 7.37 (d, J=7.9 Hz, 1H), 7.22 (d, J=8.0 Hz, 1H), 7.16 (s, 1H), 4.27-4.17 (m, 1H), 4.16-3.87 (m, 1H), 3.84-3.66 (m, 1H), 3.62-3.47 (m, 1H), 3.23-2.98 (m, 1H), 2.17 (s, 3H), 2.11 (s, 3H), 1.88-1.68 (m, 2H), 1.62-1.54 (m, 1H), 1.53-1.09 (m, 9H) ppm. ¹⁹F NMR (565 MHz, Methanol-d₄): δ −64.23 ppm.

### Step 3: Synthesis of (R)-2-(4,5-dimethyl-6-(piperidin-3-ylamino)pyridazin-3-yl)-5-(trifluoromethyl)phenol hydrochloride

In a 50 mL round bottom flask, tert-butyl (R)-3-((6-(2-hydroxy-4-(trifluoromethyl)phenyl)-4,5-dimethylpyridazin-3-yl)amino)piperidine-1-carboxylate (150 mg, 0.3 mmol, 1.0 eq.) was dissolved in 1.5 mL of 1,4-dioxane. Then, HCl (1.5 mL, 4 M in 1,4-dioxane, 6 mmol, 20 eq.) was added. The reaction mixture was stirred at rt for 2 h. Upon completion, the mixture was concentrated by rotary evaporation under reduced pressure to afford (R)-2-(4,5-dimethyl-6-(piperidin-3-ylamino)pyridazin-3-yl)-5-(trifluoromethyl)phenol hydrochloride (118 mg, assuming 100%) as white solid which was directly used in the next step without further purification. MS: mass calcd, for C₁₈H₂₁F₃N₄O 366.2, found 366.7, m/z=[M+H]⁺. ¹H NMR (600 MHz, Methanol-d₄): δ 7.57 (d, J=8.0 Hz, 1H), 7.38 (d, J=8.1 Hz, 1H), 7.31 (s, 1H), 4.47-4.40 (m, 1H), 3.65-3.61 (m, 1H), 3.42-3.36 (m, 1H), 3.12-3.02 (m, 2H), 2.47 (s, 3H), 2.29 (s, 3H), 2.25-2.19 (m, 1H), 2.16-2.10 (m, 1H), 1.96-1.85 (m, 2H) ppm. ¹⁹F NMR (564 MHz, Methanol-d₄): δ −64.79 ppm.

### Step 4: Synthesis of (R)-(3-((6-(2-hydroxy-4-(trifluoromethyl)phenyl)-4,5-dimethylpyridazin-3-yl)amino)piperidin-1-yl) (3-hydroxybicyclo[1.1.1]pentan-1-yl)methanone (67)

In a 50 mL round bottom flask, (R)-2-(4,5-dimethyl-6-(piperidin-3-ylamino)pyridazin-3-yl)-5-(trifluoromethyl)phenol hydrochloride (118 mg, 0.32 mmol, 1.0 eq.), 3-hydroxybicyclo[1.1.1]pentane-1-carboxylic acid (42 mg, 0.32 mmol, 1.0 eq.), EDAC (62 mg, 0.32 mmol, 1.0 eq.), HOBt (43 mg, 75%, containing water, 0.32 mmol, 1.0 eq.), DIPEA (0.16 mL, 3.0 eq.) were dissolved in 2 mL of DMF. The mixture was stirred at rt overnight. Upon completion, the crude material was purified by Biotage® reverse phase column chromatography (ACN/H₂O with 0.1% HCOOH). The collected fractions containing product were freeze-dried to afford the target compound (R)-(3-((6-(2-hydroxy-4-(trifluoromethyl)phenyl)-4,5-dimethylpyridazin-3-yl)amino)piperidin-1-yl) (3-hydroxybicyclo[1.1.1]pentan-1-yl)methanone (67) (103 mg, 67%) as a pale yellow solid. MS: mass calcd for C₂₄H₂₇F₃N₄O₃ 476.2, found 476.8, m/z=[M+H]⁺. ¹H NMR (600 MHz, Methanol-d₄): δ 7.43-7.37 (m, 1H), 7.27-7.21 (m, 1H), 7.19-7.14 (m, 1H), 4.57-4.43 (m, 1H), 4.30-4.20 (m, 1H), 4.19-4.06 (m, 1H), 3.22-3.03 (m, 1H), 2.99-2.82 (m, 1H), 2.34-2.28 (m, 2H), 2.27-2.21 (m, 2H), 2.19 (s, 2H), 2.18-2.13 (m, 4H), 2.11 (s, 3H), 1.90-1.83 (m, 1H), 1.82-1.75 (m, 1H), 1.65-1.54 (m, 1H) ppm. ¹⁹F NMR (564 MHz, Methanol-d₄): δ −64.26 ppm.

### Example 68: (R)-3-((3-((6-(2-Hydroxy-4-(trifluoromethyl)phenyl)-4,5-dimethylpyridazin-3-yl)amino)piperidin-1-yl)methyl) bicyclo[1.1.1]pentan-1-ol (68)

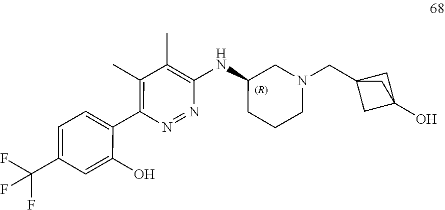

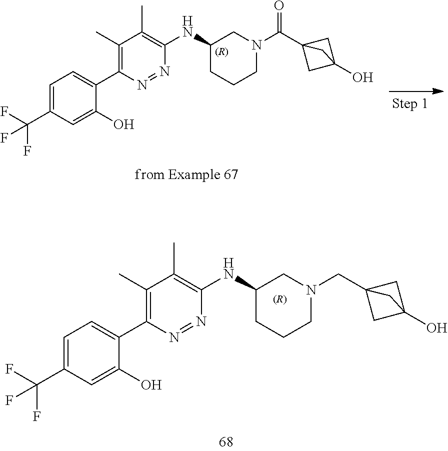

### Step 1: Synthesis of (R)-3-((3-((6-(2-hydroxy-4-(trifluoromethyl)phenyl)-4,5-dimethylpyridazin-3-yl)amino)piperidin-1-yl)methyl) bicyclo[1.1.1]pentan-1-ol (68)

In a 50 mL round bottom flask, (R)-(3-((6-(2-hydroxy-4-(trifluoromethyl)phenyl)-4,5-dimethylpyridazin-3-yl)amino)piperidin-1-yl) (3-hydroxybicyclo[1.1.1]pentan-1-yl)methanone (prepared as described for Example 67) (60 mg, 0.13 mmol, 1.0 eq.) was added. The flask was sealed with a stopper and connected with a Schlenk line to undergo N₂/vacuum cycling 3 times. 4 mL of anhydrous THF was added and the mixture was stirred at 0° C. (ice bath) under an atmosphere of nitrogen. LiAlH₄ (1 M in THF, 0.65 mL, 0.65 mmol, 5.0 eq.) was added dropwise at 0° C. (ice bath) and the mixture was heated to 60° C. (heating block) and stirred for 2 h. Upon completion, the mixture was cooled to 0° C. (ice bath), quenched with 10 mL of a saturated aqueous NH₄Cl solution and extracted with EtOAc 10 mL×3. The combined organic extracts were dried over anhydrous Na₂SO₄. After filtration, the filtrate was concentrated by rotary evaporation under reduced pressure. The crude was purified by normal phase silica gel column chromatography (5-15% MeOH in DCM) and reverse phase column chromatography (5-65% ACN/H₂O with 0.1% HCOOH). The collected fractions containing product were freeze-dried to afford the target compound (R)-3-((3-((6-(2-hydroxy-4-(trifluoromethyl)phenyl)-4,5-dimethylpyridazin-3-yl)amino)piperidin-1-yl)methyl) bicyclo[1.1.1]pentan-1-ol (68) as a pale yellow solid (11 mg, 19%). MS: mass calcd, for C₂₄H₂₉F₃N₄O₂ 462.2, found 463.2, m/z=[M+H]⁺. ¹H NMR (600 MHz, Methanol-d₄): δ 7.38 (d, J=7.9 Hz, 1H), 7.23 (dd, J=7.8, 1.0 Hz, 1H), 7.17 (d, J=2.0 Hz, 1H), 4.47-4.40 (m, 1H), 3.51-3.38 (m, 1H), 3.11-2.94 (m, 3H), 2.76-2.56 (m, 2H), 2.19 (s, 3H), 2.11 (s, 3H), 2.07-1.99 (m, 1H), 1.99-1.95 (m, 1H), 1.92 (d, J=2.1 Hz, 6H), 1.86-1.77 (m, 1H), 1.76-1.67 (m, 1H) ppm. ¹⁹F NMR (564 MHz, Methanol-d₄): δ −64.26 ppm.

### Example 69: (R)-2-(3-((6-(4-Hydroxybenzo[b]thiophen-5-yl)-5-methylpyridazin-3-yl)amino)piperidin-1-yl)ethyl 4-hydroxypiperidine-1-carboxylate (69)

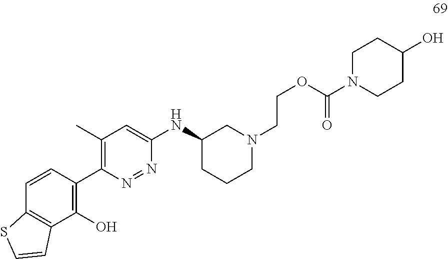 [SMILES: Cc1cc(N[C@@H]2CCCN(CCOC(=O)N3CCC(O)CC3)C2)nnc1-c1ccc2sccc2c1O]

### Synthetic Route:

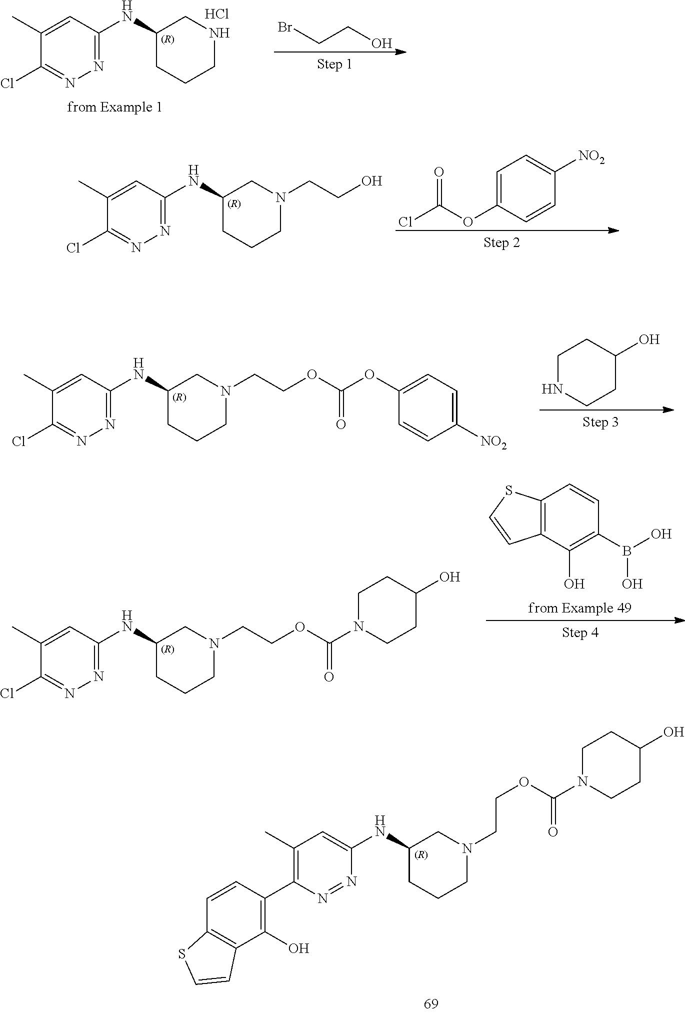

### Step 1: Synthesis of (R)-2-(3-((6-chloro-5-methylpyridazin-3-yl)amino)piperidin-1-yl)ethan-1-ol

In a single-necked flask, (R)-6-chloro-5-methyl-N-(piperidin-3-yl)pyridazin-3-amine hydrochloride (prepared as disclosed in Example 1) (950 mg, 4.2 mmol, 1.0 eq.) was dissolved in 20 mL of ACN, and then K₂CO₃ (1.7 g, 12.5 mmol, 3.0 eq.) and 2-bromo ethanol (1.0 g, 8.4 mmol, 2.0 eq.) were added. The mixture was stirred overnight at 80° C. (heating block). After the reaction mixture was cooled to rt, it was filtered through a Celite® cake and washed with 20 mL of a mixture of DCM/MeOH (v/v 50:50). The filtrate was concentrated by rotary evaporation under reduced pressure, and the residue was purified by flash column chromatography (0-20% MeOH in DCM) to afford (R)-2-(3-((6-chloro-5-methylpyridazin-3-yl)amino)piperidin-1-yl)ethan-1-ol (690 mg, 61%) as a pale yellow solid after evaporation of the combined product containing fractions under reduced pressure using a rotary evaporator. LC/MS: mass calcd for C₁₂H₁₉ClN₄O 270.12, found m/z=271.00, [M+H]⁺. ¹H NMR (400 MHz, Methanol-d₄): δ 6.78 (s, 1H), 4.07-3.99 (m, 1H), 3.68-3.62 (m, 2H), 3.02-2.89 (m, 1H), 2.71-2.59 (m, 1H), 2.51 (q, J=6.5 Hz, 2H), 2.39-2.29 (m, 1H), 2.24 (s, 3H), 2.23-2.15 (m, 1H), 1.89-1.81 (m, 1H), 1.79-1.71 (m, 1H), 1.68-1.59 (m, 1H), 1.52-1.36 (m, 1H) ppm.

### Step 2: Synthesis of Step (R)-2-(3-((6-chloro-5-methylpyridazin-3-yl)amino)piperidin-1-yl)ethyl (4-nitrophenyl) carbonate

(R)-2-(3-((6-chloro-5-methylpyridazin-3-yl)amino)piperidin-1-yl)ethan-1-ol (100 mg, 0.37 mmol, 1.0 eq.) was dissolved in 4 mL of DCM, and the solution was cooled to 0° C. (ice bath). Under an atmosphere of nitrogen, DIPEA (205 μL, 152 mg, 1.10 mmol, 3.0 eq.) and 4-nitrophenyl carbonochloridate (111 mg, 0.55 mmol, 1.5 eq.) were added. The mixture was stirred at rt for 2 hours, and the crude product (R)-2-(3-((6-chloro-5-methylpyridazin-3-yl)amino)piperidin-1-yl)ethyl (4-nitrophenyl) carbonate (150 mg, 93%) was used directly in the next step without further isolation and purification. LC/MS: mass calcd, for C₁₉H₂₂ClN₅O₅ 435.13, found m/z=436.10, [M+H]⁺.

### Step 3: Synthesis of (R)-2-(3-((6-chloro-5-methylpyridazin-3-yl)amino)piperidin-1-yl)ethyl 4-hydroxypiperidine-1-carboxylate

To a solution of (R)-2-(3-((6-chloro-5-methylpyridazin-3-yl)amino)piperidin-1-yl)ethyl (4-nitrophenyl) carbonate (150 mg crude from Step 2, ~0.34 mmol, 1.0 eq.) in 2 mL of DCM, DIPEA (146 μL, 108 mg, 1.03 mmol, 3.0 eq.) and piperidin-4-ol (35 mg, 0.34 mmol, 1.0 eq.) were added. The mixture was stirred at rt overnight, and was concentrated by rotary evaporation under reduced pressure. The residue was purified by flash column chromatography (0-20% MeOH in DCM) to afford (R)-2-(3-((6-chloro-5-methylpyridazin-3-yl)amino)piperidin-1-yl)ethyl 4-hydroxypiperidine-1-carboxylate (90 mg, 66%) as a pale yellow solid after evaporation of the combined product containing fractions under reduced pressure using a rotary evaporator. LC/MS: mass calcd for C₁₈H₂₈ClN₅O₃ 397.19, found m/z=398.05, [M+H]⁺. ¹H NMR (400 MHz, Methanol-d₄): δ 6.82 (s, 1H), 4.30-4.19 (m, 2H), 4.12-4.01 (m, 1H), 3.90-3.67 (m, 4H), 3.23-3.15 (m, 1H), 3.13-3.05 (m, 2H), 2.91-2.76 (m, 3H), 2.48 (t, J=10.4 Hz, 1H), 2.40-2.30 (m, 1H), 2.26 (s, 3H), 1.99-1.65 (m, 6H), 1.49 (d, J=10.4 Hz, 1H) ppm.

### Step 4: Synthesis of (R)-2-(3-((6-(4-hydroxybenzo[b]thiophen-5-yl)-5-methylpyridazin-3-yl)amino)piperidin-1-yl)ethyl 4-hydroxypiperidine-1-carboxylate (69)

(R)-2-(3-((6-chloro-5-methylpyridazin-3-yl)amino)piperidin-1-yl)ethyl 4-hydroxypiperidine-1-carboxylate (90 mg, 0.23 mmol, 1.0 eq.), (4-hydroxybenzo[b]thiophen-5-yl) boronic acid (53 mg, 0.27 mmol, 1.2 eq.), sodium carbonate (71 mg, 0.62 mmol, 3.0 eq.), and tetrakis(triphenylphosphine) palladium (0) Pd((PPh)₃)₄ (26 mg, 0.022 mmol, 0.1 eq.) were added in 3 mL of 1,4-dioxane and 0.5 mL of water under an atmosphere of nitrogen. The mixture was heated in a sealed tube at 110° C. (heating block) for 16 hours. The reaction tube was cooled to room temperature, and then 10 mL of water was added. The mixture was extracted with 3×10 mL of EtOAc, and the combined organic layers were dried over sodium sulfate, filtered, and concentrated by rotary evaporation under reduced pressure. The residue was purified by preparative RP-HPLC (10-70% ACN/water with 0.1% HCOOH) and lyophilized to afford the target compound (R)-2-(3-((6-(4-hydroxybenzo[b]thiophen-5-yl)-5-methylpyridazin-3-yl)amino)piperidin-1-yl)ethyl 4-hydroxypiperidine-1-carboxylate (69) (31 mg, 27%) as a yellow solid after lyophilization of the combined product containing fractions. LC/MS: mass calcd, for C₂₆H₃₃N₅O₄S 511.23, found m/z=512.20, [M+H]⁺. ¹H NMR (400 MHz, Methanol-d₄): δ 8.39 (s, 1H), 7.58 (d, J=5.5 Hz, 1H), 7.50 (d, J=6.5 Hz, 2H), 7.22 (d, J=8.3 Hz, 1H), 6.87 (s, 1H), 4.34 (brs, 2H), 4.28-4.19 (m, 1H), 3.86-3.68 (m, 3H), 3.55 (brs, 1H), 3.16-3.00 (m, 5H), 2.85-2.61 (m, 2H), 2.19 (s, 3H), 2.08-1.91 (m, 2H), 1.89-1.55 (m, 4H), 1.35 (brs, 2H) ppm.

### Example 70: ((R)-3-((6-(2-Hydroxy-4-(trifluoromethyl)phenyl)-5-methylpyridazin-3-yl)amino)piperidin-1-yl) (3-hydroxycyclohexyl)methanone (70)—Fraction A and Fraction B

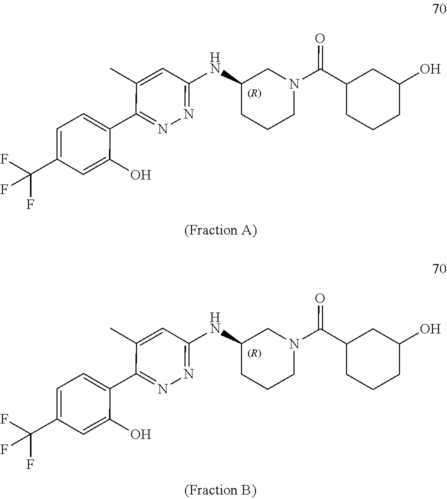

### Synthetic Route:

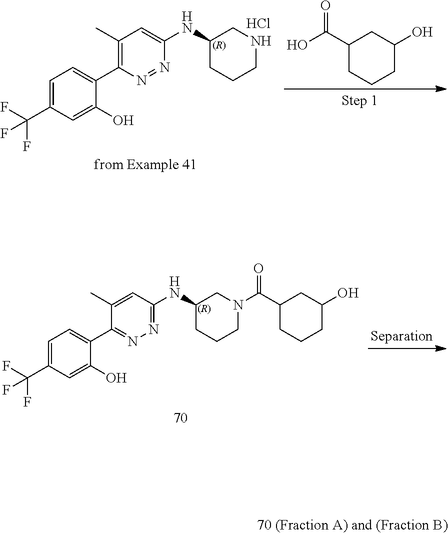

### Step 1: Synthesis of ((R)-3-((6-(2-hydroxy-4-(trifluoromethyl)phenyl)-5-methylpyridazin-3-yl)amino)piperidin-1-yl) (3-hydroxycyclohexyl)methanone (70) Fraction A and (70) Fraction B

In a 50 mL round bottom flask, 3-hydroxycyclohexane-1-carboxylic acid (111 mg, 0.8 mmol, 1.0 eq.), HATU (323 mg, 0.9 mmol, 1.1 eq.), and DIPEA (0.27 mL, 200 mg, 1.5 mmol, 2.0 eq.) were dissolved in 10 mL of DMF. The mixture was stirred at rt for 1 hour, before addition of the (R)-2-(4-methyl-6-(piperidin-3-ylamino)pyridazin-3-yl)-5-(trifluoromethyl)phenol hydrochloride (prepared as disclosed in Example 41) (300 mg, 0.8 mmol, 1.0 eq.). The resulting mixture was stirred at rt for 2 hours. The mixture was then concentrated by rotary evaporation under reduced pressure and dissolved in 20 mL of EtOAc. The mixture was washed with 2×20 mL of a 5 wt-% aqueous LiCl solution, 2×20 mL of brine, and dried over anhydrous Na₂SO₄. After filtration, the filtrate was concentrated by rotary evaporation under reduced pressure. The crude material was further purified by preparative RP-HPLC (5-65% ACN in H₂O with 0.1% HCOOH in 30 min) to afford two fractions of the target compound ((R)-3-((6-(2-hydroxy-4-(trifluoromethyl)phenyl)-5-methylpyridazin-3-yl)amino)piperidin-1-yl) (3-hydroxycyclohexyl)methanone (70). Fraction A: (42 mg, 12%, first eluting) and Fraction B (29 mg, 8%, second eluting) both as white solids after lyophilization of the their respective product containing fractions.

### 70 Fraction A (First Eluting):

MS: mass calcd for C₂₄H₂₉F₃N₄O₃ 478.22, found 479.10, m/z=[M+H]⁺. ¹H NMR (400 MHz, Methanol-d₄): δ 7.45-7.38 (m, 1H), 7.26-7.21 (m, 1H), 7.19-7.14 (m, 1H), 6.82-6.78 (m, 1H), 4.41-4.25 (m, 1H), 4.15-4.10 (m, 1H), 4.09-3.98 (m, 1H), 3.97-3.81 (m, 1H), 3.28-3.19 (m, 1H), 3.18-3.00 (m, 2H), 2.29-2.19 (m, 1H), 2.14 (s, 3H), 1.95-1.82 (m, 1H), 1.81-1.62 (m, 4H), 1.61-1.38 (m, 6H) ppm. ¹⁹F NMR (376 MHz, Methanol-d₄): δ −64.33 ppm.

### 70 Fraction B (Second Eluting):

MS: mass calcd for C₂₄H₂₉F₃N₄O₃ 478.22, found 479.10, m/z=[M+H]⁺. ¹H NMR (400 MHz, Methanol-d₄): δ 7.41 (t, J=8.8 Hz, 1H), 7.23 (d, J=8.1 Hz, 1H), 7.17 (s, 1H), 6.81 (s, 1H), 4.34-4.24 (m, 1H), 4.20-4.10 (m, 1H), 4.10-3.92 (m, 2H), 3.29-3.20 (m, 1H), 3.18-3.03 (m, 2H), 2.14 (s, 3H), 1.98-1.78 (m, 2H), 1.77-1.74 (m, 1H), 1.74-1.61 (m, 4H), 1.61-1.42 (m, 4H), 1.41-1.23 (m, 1H) ppm. ¹⁹F NMR (376 MHz, Methanol-d₄): δ −64.33 ppm.

### Example 71: 2-(6-(((3R)-1-((3-Hydroxycyclohexyl)methyl)piperidin-3-yl)amino)-4-methylpyridazin-3-yl)-5-(trifluoromethyl)phenol (71)

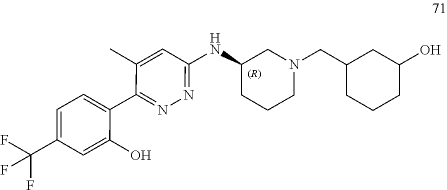

### Synthetic Route:

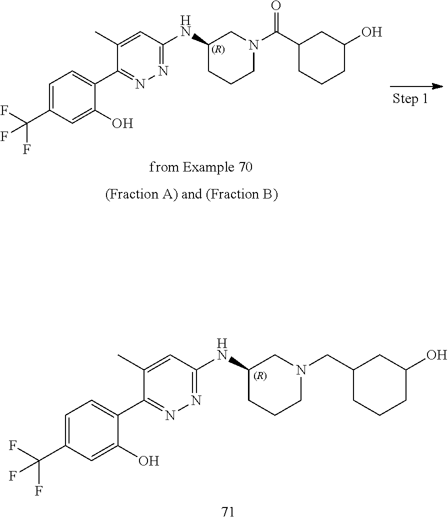

### Step 1: Synthesis of 2-(6-(((3R)-1-((3-hydroxycyclohexyl)methyl)piperidin-3-yl)amino)-4-methylpyridazin-3-yl)-5-(trifluoromethyl)phenol (71)

To a 50 mL oven-dried round bottom flask, LAH (80 mg, 2.1 mmol, 10.0 eq.) was added. The round bottom flask was sealed with a rubber septum stopper, connected to a Schlenk line to undergo nitrogen/vacuum cycling three times, and then connected to a nitrogen balloon. 10 mL of anhydrous THF was added into the flask at 0° C. (ice bath). A mixture of ((R)-3-((6-(2-hydroxy-4-(trifluoromethyl)phenyl)-5-methylpyridazin-3-yl)amino)piperidin-1-yl) (3-hydroxycyclohexyl)methanone (70), Fraction A (first eluting) and Fraction B (second eluting) (prepared as disclosed in Example 70) (100 mg, 0.2 mmol, 1.0 eq.) was dissolved in 5 mL of anhydrous THF, and then added into the flask dropwise. The resulting mixture was heated to 60° C. (heating block) for 2 hours. Once the reaction was completed, the mixture was cooled to 0° C. (ice bath). 1 mL of DI water, 1 mL of an aqueous 10 wt-% NaOH solution, and 8 mL of DI water were added sequentially to quench the reaction. The mixture was extracted with 3×10 mL EtOAc. The combined organic extracts were dried over anhydrous Na₂SO₄, filtered, and concentrated by rotary evaporation under reduced pressure. The crude was further purified by preparative RP-HPLC (5-65% ACN in H₂O with 0.1% HCOOH) to afford the target compound 2-(6-(((3R)-1-((3-hydroxycyclohexyl)methyl)piperidin-3-yl)amino)-4-methylpyridazin-3-yl)-5-(trifluoromethyl)phenol (71) (32 mg, 33%) as a pale yellow solid after lyophilization of the combined product containing fractions. MS: mass calcd for C₂₄H₃₁F₃N₄O₂ 464.24, found 465.10, m/z=[M+H]⁺. ¹H NMR (400 MHz, Methanol-d₄): δ 7.39 (d, J=7.9 Hz, 1H), 7.24 (d, J=7.9 Hz, 1H), 7.17 (s, 1H), 6.81 (s, 1H), 4.36-4.22 (m, 1H), 4.08-3.98 (m, 1H), 3.67-3.43 (m, 1H), 3.28-3.12 (m, 1H), 2.89-2.46 (m, 4H), 2.28-2.17 (m, 1H), 2.14 (s, 3H), 2.11-1.96 (m, 2H), 1.95-1.58 (m, 6H), 1.57-1.42 (m, 2H), 1.36-1.20 (m, 1H), 1.16-0.96 (m, 1H) ppm. ¹⁹F NMR (376 MHz, Methanol-d₄): δ −64.33 ppm.

### Example 72: (R)-2-(6-((1-(2-(4-Hydroxycyclohexyl)ethyl)piperidin-3-yl)amino)-4-methylpyridazin-3-yl)-5-(trifluoromethyl)phenol (72) Fraction A and Fraction B

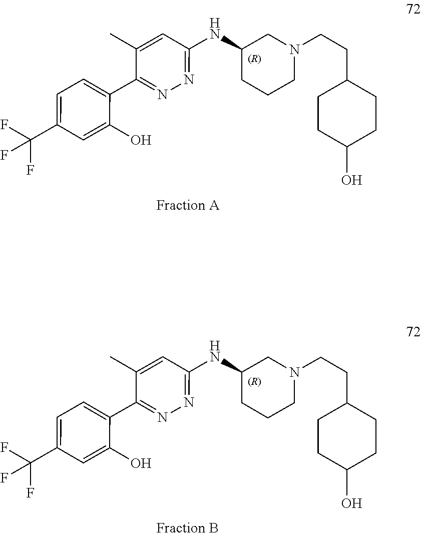

### Synthetic Route:

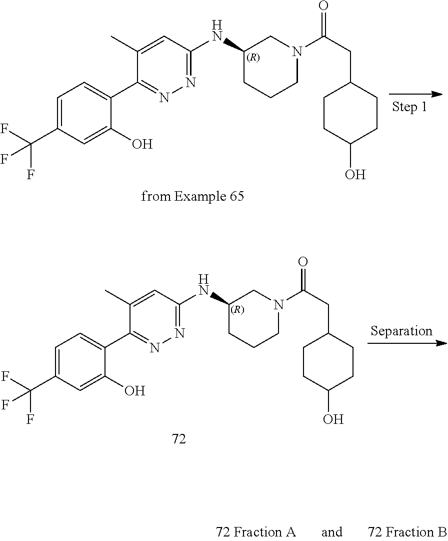

### Step 1: Synthesis of (R)-2-(6-((1-(2-(4-hydroxycyclohexyl)ethyl)piperidin-3-yl)amino)-4-methylpyridazin-3-yl)-5-(trifluoromethyl)phenol (72) Fraction A and Fraction B

To a solution of LAH (135 mg, 3.0 mmol, 10 eq.) in anhydrous THF (20 mL), (R)-1-(3-((6-(2-hydroxy-4-(trifluoromethyl)phenyl)-5-methylpyridazin-3-yl)amino)piperidin-1-yl)-2-(4-hydroxycyclohexyl)ethan-1-one (prepared as disclosed in Example 65) (150 mg, 0.3 mmol, 1.0 eq.) was added under an atmosphere of nitrogen, and the mixture was stirred at 60° C. (heating block) for 2 hours. The reaction was cooled to 0° C. (ice bath), and then slowly quenched with 5 mL of methanol. The mixture was filtered and concentrated by rotary evaporation under reduced pressure. The residue was purified by preparative RP-HPLC (5-65% ACN/water with 0.1% formic acid) and the combined product containing fractions were lyophilized to afford the target compound (R)-2-(6-((1-(2-(4-hydroxycyclohexyl)ethyl)piperidin-3-yl)amino)-4-methylpyridazin-3-yl)-5-(trifluoromethyl)phenol (72) as two fractions—Fraction A (20 mg, 14%, first fraction) as a yellow solid and Fraction B (13 mg, 9%, second fraction) as a white solid.

Fraction A (first fraction): LC/MS: mass calcd for C₂₅H₃₃F₃N₄O₂ 478.26, found m/z=479.15, [M+H]⁺. ¹H NMR (400 MHz, Methanol-d₄): δ 7.37 (d, J=7.8 Hz, 1H), 7.22 (d, J=7.8 Hz, 1H), 7.15 (s, 1H), 6.79 (s, 1H), 4.30-4.18 (m, 1H), 3.55 (s, 1H), 3.48-3.39 (m, 1H), 3.26-3.15 (m, 1H), 2.96-2.85 (m, 2H), 2.83-2.49 (m, 2H), 2.12 (s, 3H), 2.06-1.98 (m, 1H), 1.96-1.87 (m, 2H), 1.87-1.69 (m, 3H), 1.67-1.49 (m, 3H), 1.33-1.15 (m, 4H), 1.10-0.93 (m, 2H) ppm. ¹⁹F NMR (376 MHz, Methanol-d₄): δ −64.34 ppm.

Fraction B (second fraction): LC/MS: mass calcd for C₂₅H₃₃F₃N₄O₂ 478.26, found m/z=479.15, [M+H]⁺. ¹H NMR (400 MHz, Methanol-d₄): δ 7.39 (d, J=7.8 Hz, 1H), 7.23 (d, J=7.8 Hz, 1H), 7.17 (s, 1H), 6.80 (s, 1H), 4.31-4.20 (m, 1H), 3.91-3.84 (m, 1H), 3.62-3.44 (m, 1H), 3.28-3.11 (m, 1H), 2.93-2.83 (m, 2H), 2.81-2.45 (m, 2H), 2.14 (s, 3H), 2.12-2.04 (m, 1H), 2.04-1.97 (m, 1H), 1.89-1.78 (m, 1H), 1.74-1.66 (m, 2H), 1.63-1.53 (m, 4H), 1.52-1.39 (m, 5H), 1.32-1.28 (m, 1H) ppm. ¹⁹F NMR (376 MHz, Methanol-d₄): δ −64.33 ppm.

### Example 73: 5-(6-(((R)-1-(((1s,3S)-3-Hydroxycyclobutyl)methyl)piperidin-3-yl)amino)-4,5-dimethylpyridazin-3-yl)benzo[b]thiophen-4-ol (73)

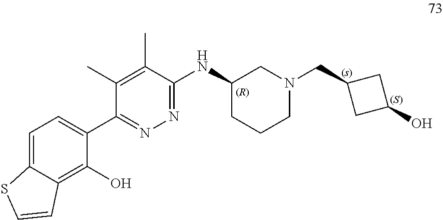

### Synthetic Route:

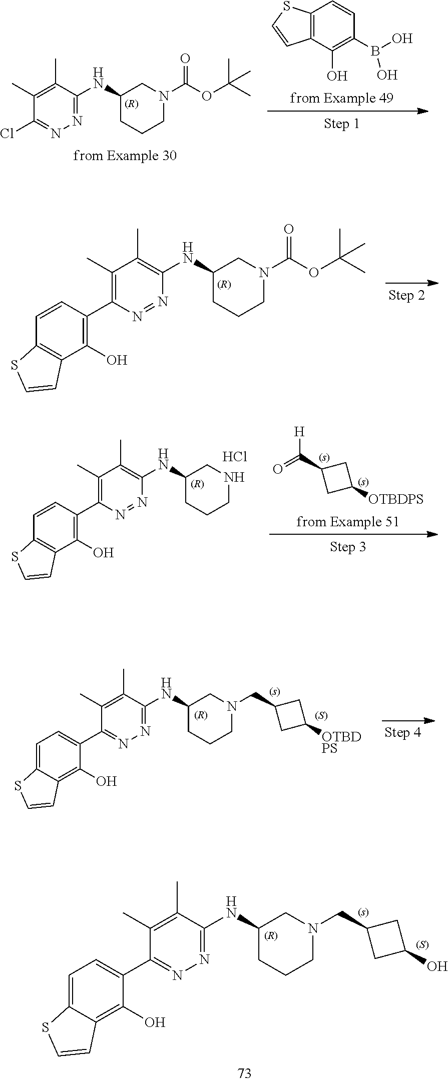

### Step 1: Synthesis of tert-butyl (R)-3-((6-(4-hydroxybenzo[b]thiophen-5-yl)-4,5-dimethylpyridazin-3-yl)amino)piperidine-1-carboxylate

In a 50 mL sealed tube, tert-butyl (R)-3-((6-chloro-4,5-dimethylpyridazin-3-yl)amino)piperidine-1-carboxylate (prepared as disclosed in Example 30) (400 mg, 1.2 mmol, 1.0 eq.), (4-hydroxybenzo[b]thiophen-5-yl) boronic acid (prepared as disclosed in Example 49) (342 mg, 1.8 mmol, 1.5 eq.), Pd(PPh₃)₄ (136 mg, 0.12 mmol, 0.1 eq.), and Na₂CO₃ (373 mg, 3.5 mmol, 3.0 eq.) were added. The tube was sealed and connected to a Schlenk line to undergo nitrogen/vacuum cycling three times. 10 mL of 1,4-dioxane and 2 mL of DI water were added to the tube. The mixture was stirred at 110° C. (heating block) for 16 hours. Once the reaction was complete, 20 mL of DI water was added, and the mixture was extracted with 3×30 mL of EtOAc. The combined organic extracts were dried over anhydrous Na₂SO₄, filtered, and concentrated by rotary evaporation under reduced pressure. The crude material was purified by flash column chromatography (0-60% EA in hexane) to afford tert-butyl (R)-3-((6-(4-hydroxybenzo[b]thiophen-5-yl)-4,5-dimethylpyridazin-3-yl)amino)piperidine-1-carboxylate (280 mg, 52%) as a yellow solid after evaporation of the combined product containing fractions under reduced pressure using a rotary evaporator.

MS: mass calcd for C₂₄H₃₀N₄O₃S 454.20, found 455.05, m/z=[M+H]⁺. ¹H NMR (400 MHz, Chloroform-d): δ 7.59-7.50 (d, J=5.4 Hz, 1H), 7.40 (d, J=8.4 Hz, 1H), 7.34 (d, J=5.5 Hz, 1H), 7.24 (d, J=8.6 Hz, 1H), 4.39-4.27 (m, 1H), 3.84-3.44 (m, 3H), 3.35-3.15 (m, 1H), 2.35 (s, 3H), 2.13 (s, 3H), 2.00-1.79 (m, 2H), 1.79-1.66 (m, 1H), 1.65-1.53 (m, 1H), 1.42 (s, 9H) ppm.

### Step 2: Synthesis of (R)-5-(4,5-dimethyl-6-(piperidin-3-ylamino)pyridazin-3-yl)benzo[b]thiophen-4-ol hydrochloride

In a 50 mL round bottom flask, tert-butyl (R)-3-((6-(4-hydroxybenzo[b]thiophen-5-yl)-4,5-dimethylpyridazin-3-yl)amino)piperidine-1-carboxylate (280 mg, 0.6 mmol, 1.0 eq.) was dissolved in 2 mL of 1,4-dioxane, and then HCl (2.0 mL, 4 M in dioxane, 8 mmol, 13.0 eq.) was added. The mixture was stirred at rt for 1 hour. The mixture was concentrated by rotary evaporation under reduced pressure and purified by flash column chromatography (0-20% MeOH in DCM) to afford (R)-5-(4,5-dimethyl-6-(piperidin-3-ylamino)pyridazin-3-yl)benzo[b]thiophen-4-ol hydrochloride (180 mg, 82%) as a yellow solid after evaporation of the combined product containing fractions under reduced pressure using a rotary evaporator. MS: mass calcd for C₁₉H₂₂N₄OS 354.15, found 354.95, m/z=[M+H]⁺. ¹H NMR (400 MHz, Methanol-d₄): δ 7.66 (d, J=5.7 Hz, 1H), 7.64-7.60 (m, 2H), 7.25 (d, J=8.3 Hz, 1H), 4.52-4.41 (m, 1H), 3.70-3.61 (m, 1H), 3.41-3.37 (m, 1H), 3.10-2.99 (m, 2H), 2.41 (s, 3H), 2.27 (s, 3H), 2.25-2.18 (m, 1H), 2.17-2.09 (m, 1H), 1.97-1.83 (m, 2H) ppm.

### Step 3: Synthesis of 5-(6-(((R)-1-(((1s,3S)-3-((tert-butyldiphenylsilyl)oxy)cyclobutyl)methyl)piperidin-3-yl)amino)-4,5-dimethylpyridazin-3-yl)benzo[b]thiophen-4-ol

In a 50 mL round bottom flask, (R)-5-(4,5-dimethyl-6-(piperidin-3-ylamino)pyridazin-3-yl)benzo[b]thiophen-4-ol hydrochloride (90 mg, 0.3 mmol, 1.0 eq.) and (1s,3s)-3-((tert-butyldiphenylsilyl)oxy)cyclobutane-1-carbaldehyde (prepared as disclosed in Example 51) (172 mg, 0.5 mmol, 2.0 eq.) were dissolved in 5 mL of MeOH, and acetic acid (91 mg, 1.5 mmol, 6.0 eq.) was added. The mixture was stirred at rt for 1 hour. The mixture was cooled to 0° C. (ice bath) before the addition of NaBH₃CN (48 mg, 0.8 mmol, 3.0 eq.). The mixture was warmed to rt and stirred for 2 hours. Once the reaction was complete, the mixture was cooled to 0° C. (ice bath), quenched with 20 mL of a saturated aqueous NaHCO₃ solution, and extracted with 3×10 mL of EtOAc. The combined organic extracts were dried over anhydrous Na₂SO₄, filtered, and concentrated by rotary evaporation under reduced pressure. The crude material was further purified by flash column chromatography (0-4% MeOH in DCM) to afford 5-(6-(((R)-1-(((1s,3S)-3-((tert-butyldiphenylsilyl)oxy)cyclobutyl)methyl)piperidin-3-yl)amino)-4,5-dimethylpyridazin-3-yl)benzo[b]thiophen-4-ol (170 mg, 99%) as a yellow solid after evaporation of the combined product containing fractions under reduced pressure using a rotary evaporator. MS: mass calcd for C₄₀H₄₈N₄O₂SSi 676.33, found 677.35, m/z=[M+H]⁺. ¹H NMR (400 MHz, Methanol-d₄): δ 7.68-7.61 (m, 4H), 7.60 (d, J=5.7 Hz, 1H), 7.51 (s, 1H), 7.49 (d, J=2.8 Hz, 1H), 7.45-7.32 (m, 6H), 7.17 (d, J=8.4 Hz, 1H), 4.47-4.34 (m, 1H), 4.17-4.10 (m, 1H), 3.47 (d, J=5.1 Hz, 1H), 3.46-3.37 (m, 1H), 3.08-2.96 (m, 1H), 2.91-2.80 (m, 2H), 2.70-2.47 (m, 2H), 2.41-2.29 (m, 2H), 2.19 (s, 3H), 2.15 (s, 3H), 2.11-1.99 (m, 1H), 1.98-1.94 (m, 1H), 1.84-1.73 (m, 3H), 1.72-1.60 (m, 1H), 1.00 (s, 9H) ppm.

### Step 4: Synthesis of 5-(6-(((R)-1-(((1s,3S)-3-hydroxycyclobutyl)methyl)piperidin-3-yl)amino)-4,5-dimethylpyridazin-3-yl)benzo[b]thiophen-4-ol (73)

In a 50 mL round bottom flask, 5-(6-(((R)-1-(((1s,3S)-3-((tert-butyldiphenylsilyl)oxy)cyclobutyl)methyl)piperidin-3-yl)amino)-4,5-dimethylpyridazin-3-yl)benzo[b]thiophen-4-ol (170 mg, 0.3 mmol, 1.0 eq.) was dissolved in 5 mL of THF before addition of TBAF (0.5 mL, 1.0M in THF, 0.50 mmol, 2.0 eq.). The mixture was stirred at rt overnight. Once the reaction was complete, the mixture was diluted with 10 mL of EtOAc and washed with 2×30 mL of a saturated aqueous NaHCO₃ solution. The combined organic extracts were dried over anhydrous Na₂SO₄, filtered, and concentrated by rotary evaporation under reduced pressure. The crude material was further purified by flash column chromatography (0-10% MeOH in DCM) to afford the title compound 5-(6-(((R)-1-(((1s,3S)-3-Hydroxycyclobutyl)methyl)piperidin-3-yl)amino)-4,5-dimethylpyridazin-3-yl)benzo[b]thiophen-4-ol (73) (32 mg, 29%) as a yellow solid after evaporation of the combined product containing fractions under reduced pressure using a rotary evaporator.

MS: mass calcd for C₂₄H₃₀N₄O₂S 438.21, found 439.05, m/z=[M+H]⁺. ¹H NMR (400 MHz, Methanol-d₄): δ 7.56 (d, J=5.8 Hz, 1H), 7.51-7.45 (m, 2H), 7.16 (d, J=8.3 Hz, 1H), 4.48-4.38 (m, 1H), 4.10-3.99 (m, 1H), 3.44-3.32 (m, 1H), 3.02-2.90 (m, 1H), 2.84-2.74 (m, 2H), 2.66-2.51 (m, 2H), 2.50-2.40 (m, 2H), 2.18 (s, 3H), 2.13 (s, 3H), 2.11-1.99 (m, 2H), 1.97-1.91 (m, 1H), 1.85-1.72 (m, 1H), 1.72-1.54 (m, 3H) ppm.

### Example 74:5-(6-(((R)-1-(((1r,3R)-3-Hydroxycyclobutyl)methyl)piperidin-3-yl)amino)-4,5-dimethylpyridazin-3-yl)benzo[b]thiophen-4-ol (74)

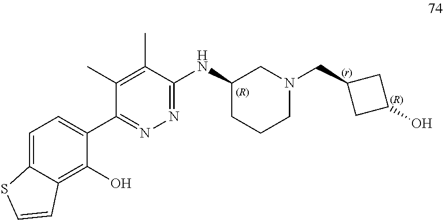

### Synthetic Route:

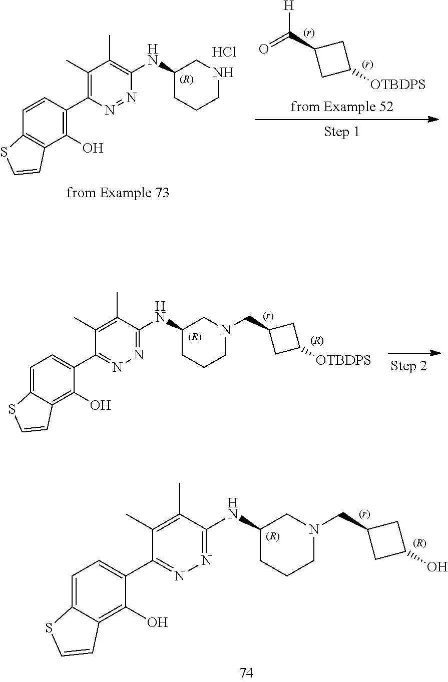

### Step 1. Synthesis of 5-(6-(((R)-1-(((1r,3R)-3-((tert-butyldiphenylsilyl)oxy)cyclobutyl)methyl)piperidin-3-yl)amino)-4,5-dimethylpyridazin-3-yl)benzo[b]thiophen-4-ol

In a single-necked flask, (R)-5-(4,5-dimethyl-6-(piperidin-3-ylamino)pyridazin-3-yl)benzo[b]thiophen-4-ol (prepared as disclosed in Example 73) (80 mg, 0.23 mmol, 1.0 eq.) and (1r,3r)-3-((tert-butyldiphenylsilyl)oxy)cyclobutane-1-carbaldehyde (prepared as disclosed in Example 52) (153 mg, 0.45 mmol, 2.0 eq.) were dissolved in 5 mL of MeOH, and acetic acid (89 mg, 1.5 mmol, 6.0 eq.) was then added under an atmosphere of nitrogen. The mixture was stirred at rt for 1 hour, and then cooled to 0° C. (ice bath). Sodium cyanoborohydride (28 mg, 0.45 mmol, 2.0 eq.) was added, and the reaction was stirred at rt for 2 hours. The reaction mixture was cooled to 0° C. (ice bath), and 20 mL of a saturated aqueous NaHCO₃ solution was added to quench the reaction. The mixture was extracted with 3×10 mL of EtOAc. The organic layers were combined, dried over sodium sulfate, filtered, and concentrated by rotary evaporation under reduced pressure. The residue was subjected to flash column chromatography (0-4% MeOH in DCM) to afford 5-(6-(((R)-1-(((1r,3R)-3-((tert-butyldiphenylsilyl)oxy)cyclobutyl)methyl)piperidin-3-yl)amino)-4,5-dimethylpyridazin-3-yl)benzo[b]thiophen-4-ol (140 mg, 92%) as a yellow solid after evaporation of the combined product containing fractions under reduced pressure using a rotary evaporator. LC/MS: mass calcd for C₄₀H₄₈N₄O₂SSi 676.33, found m/z=677.35, [M+H]⁺. ¹H NMR (400 MHz, Methanol-d₄): δ 7.64-7.60 (m, 4H), 7.57 (d, J=5.2 Hz, 1H), 7.50-7.45 (m, 2H), 7.41-7.32 (m, 6H), 7.17-7.14 (m, 1H), 4.44-4.30 (m, 1H), 3.21-3.05 (m, 1H), 2.66-2.50 (m, 2H), 2.26 (s, 2H), 2.19-2.10 (m, 5H), 2.06-1.97 (m, 2H), 1.90 (s, 1H), 1.87-1.77 (m, 1H), 1.77-1.58 (m, 2H), 1.34-1.24 (m, 1H), 0.99 (s, 9H) ppm.

### Step 2: Synthesis of 5-(6-(((R)-1-(((1r,3R)-3-hydroxycyclobutyl)methyl)piperidin-3-yl)amino)-4,5-dimethylpyridazin-3-yl)benzo[b]thiophen-4-ol (74)

To a solution of 5-(6-(((R)-1-(((1r,3R)-3-((tert-butyldiphenylsilyl)oxy)cyclobutyl)methyl)piperidin-3-yl)amino)-4,5-dimethylpyridazin-3-yl)benzo[b]thiophen-4-ol (140 mg, 0.2 mmol, 1.0 eq.) in THF (2 mL), tetrabutylammonium fluoride (1 M in THF, 0.4 mL, 0.4 mmol, 2.0 eq.) was added, and the mixture was stirred at rt overnight. The reaction mixture was diluted with 10 mL of EtOAc, and then washed with 2×10 mL of saturated aqueous NaHCO₃ solution. The organic extracts were dried over sodium sulfate, filtered, and the filtrate was concentrated by rotary evaporation under reduced pressure. The residue was subjected to flash column chromatography purification (0-10% MeOH in DCM) to afford the title compound 5-(6-(((R)-1-(((1r,3R)-3-hydroxycyclobutyl)methyl)piperidin-3-yl)amino)-4,5-dimethylpyridazin-3-yl)benzo[b]thiophen-4-ol (74) (20 mg, 22%) as a yellow solid after evaporation of the combined product containing fractions under reduced pressure using a rotary evaporator. LC/MS: mass calcd for C₂₄H₃₀N₄O₂S 438.21, found m/z=439.05, [M+H]⁺. ¹H NMR (400 MHz, Methanol-d₄): δ 7.57 (d, J=5.5 Hz, 1H), 7.49 (d, J=6.1 Hz, 2H), 7.16 (d, J=8.1 Hz, 1H), 4.48-4.42 (m, 1H), 4.29 (p, J=6.4 Hz, 1H), 3.43-3.34 (m, 1H), 3.04-2.95 (m, 1H), 2.88-2.83 (m, 2H), 2.72-2.40 (m, 4H), 2.18 (s, 3H), 2.13 (s, 3H), 2.12-2.06 (m, 4H), 1.98-1.92 (m, 1H), 1.85-1.76 (m, 1H), 1.73-1.64 (m, 1H) ppm.

### Example 75: (R)-2-(3-((6-(2-Hydroxy-4-(trifluoromethyl)phenyl)-4,5-dimethylpyridazin-3-yl)amino)piperidin-1-yl)ethyl 4-hydroxypiperidine-1-carboxylate (75)

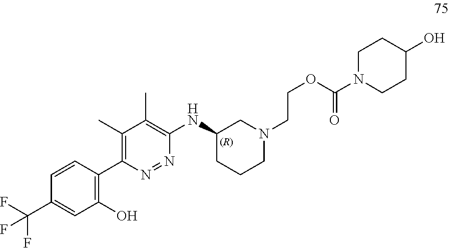

### Synthetic Route:

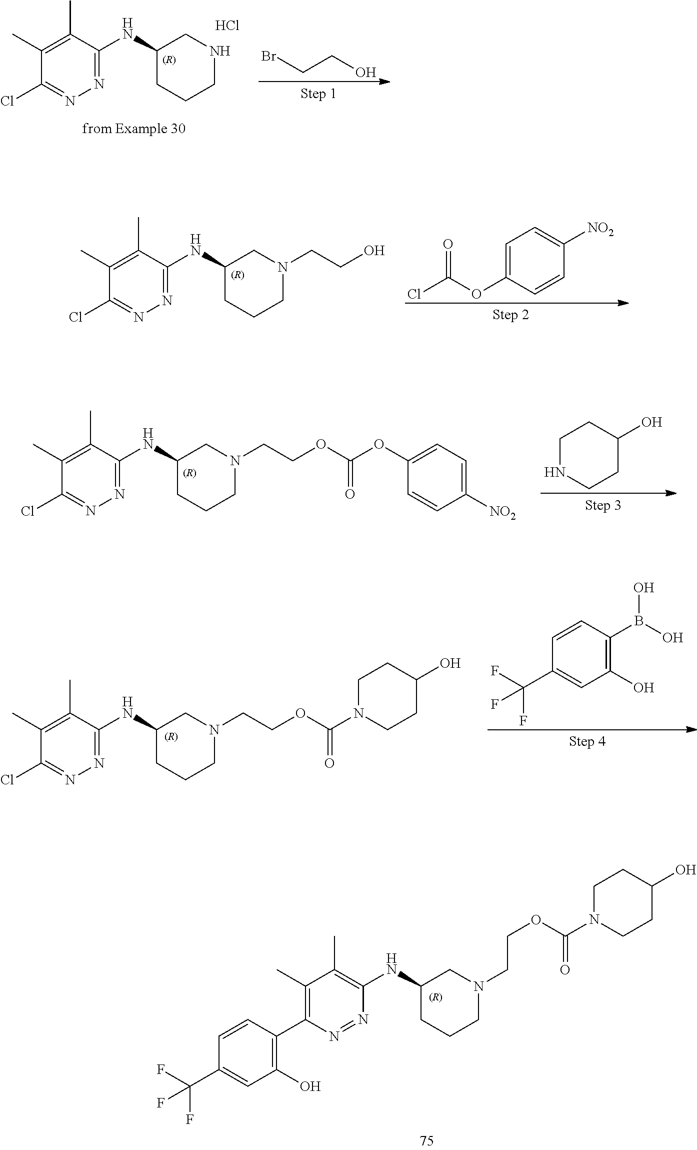

### Step 1: Synthesis of (R)-2-(3-((6-chloro-4,5-dimethylpyridazin-3-yl)amino)piperidin-1-yl)ethan-1-ol

In a 100 mL round bottom flask, (R)-6-chloro-4,5-dimethyl-N-(piperidin-3-yl)pyridazin-3-amine hydrochloride (prepared as disclosed in Example 30) (1.0 g, 3.6 mmol, 1.0 eq.), 2-bromoethan-1-ol (1.4 g, 10.8 mmol, 3.0 eq.), and K₂CO₃ (1.5 g, 10.8 mmol, 3.0 eq.) were mixed in 30 mL of ACN. The mixture was stirred at 80° C. (heating block) overnight. The mixture was then cooled to room temperature, filtered over Celite®, and washed with DCM/MeOH followed by drying over sodium sulfate, filtration, and evaporation of the filtrate under reduced pressure using a rotary evaporator. The crude material was further purified by flash column chromatography (0-20% MeOH in DCM) to afford (R)-2-(3-((6-chloro-4,5-dimethylpyridazin-3-yl)amino)piperidin-1-yl)ethan-1-ol (520 mg, 51%) as a pale yellow solid after evaporation of the combined product containing fractions under reduced pressure using a rotary evaporator. MS: mass calcd for C₁₃H₂₁ClN₄O 284.14, found 284.95, m/z=[M+H]⁺. ¹H NMR (400 MHz, Chloroform-d): δ 4.79 (br s, 1H), 4.58-4.41 (m, 1H), 3.81-3.73 (m, 1H), 3.71-3.59 (m, 2H), 2.91-2.77 (m, 1H), 2.76-2.54 (m, 3H), 2.53-2.42 (m, 1H), 2.30 (s, 3H), 2.11 (s, 3H), 1.92-1.57 (m, 4H) ppm.

### Step 2: Synthesis of (R)-2-(3-((6-chloro-4,5-dimethylpyridazin-3-yl)amino)piperidin-1-yl)ethyl (4-nitrophenyl) carbonate

In a 50 mL round bottom flask, (R)-2-(3-((6-chloro-4,5-dimethylpyridazin-3-yl)amino)piperidin-1-yl)ethan-1-ol (200 mg, 0.7 mmol, 1.0 eq.) was dissolved in 10 mL of DCM. The mixture was cooled to 0° C. (ice bath), before addition of 4-nitrophenyl carbonochloridate (213 mg, 1.1 mmol, 1.5 eq.) and DIPEA (0.37 ml, 275 mg, 2.1 mmol, 3.0 eq.). The mixture was warmed to rt and stirred for 2 hours. The crude was concentrated by rotary evaporation under reduced pressure to afford (R)-2-(3-((6-chloro-4,5-dimethylpyridazin-3-yl)amino)piperidin-1-yl)ethyl (4-nitrophenyl) carbonate as a yellow solid (280 mg, 89%) which was used in the next step without further isolation, purification, and characterization. MS: mass calcd for C₂₀H₂₄ClN₅O₅ 449.15, found 450.00, m/z=[M+H]⁺.

### Step 3: Synthesis of (R)-2-(3-((6-chloro-4,5-dimethylpyridazin-3-yl)amino)piperidin-1-yl)ethyl 4-hydroxypiperidine-1-carboxylate

In a 50 mL round bottom flask, (R)-2-(3-((6-chloro-4,5-dimethylpyridazin-3-yl)amino)piperidin-1-yl)ethyl (4-nitrophenyl) carbonate (280 mg, 0.6 mmol, 1.0 eq.), piperidin-4-ol (126 mg, 1.2 mmol, 2.0 eq.), and DIPEA (0.33 mL, 245 mg, 1.9 mmol, 3.0 eq.) were dissolved in 10 mL of DCM. The mixture was stirred at rt for 1 hour. The crude reaction product was concentrated by rotary evaporation under reduced pressure and further purified by flash column chromatography (0-20% MeOH in DCM) to afford (R)-2-(3-((6-chloro-4,5-dimethylpyridazin-3-yl)amino)piperidin-1-yl)ethyl 4-hydroxypiperidine-1-carboxylate (200 mg, 78%) as a pale yellow solid after evaporation of the combined product containing fractions under reduced pressure using a rotary evaporator. MS: mass calcd for C₁₉H₃₀ClN₅O₃ 411.20, found 412.05, m/z=[M+H]⁺.

### Step 4: Synthesis of (R)-2-(3-((6-(2-hydroxy-4-(trifluoromethyl)phenyl)-4,5-dimethylpyridazin-3-yl)amino)piperidin-1-yl)ethyl 4-hydroxypiperidine-1-carboxylate (75)

In a 20 mL sealed tube, (R)-2-(3-((6-chloro-4,5-dimethylpyridazin-3-yl)amino)piperidin-1-yl)ethyl 4-hydroxypiperidine-1-carboxylate (200 mg, 0.49 mmol, 1.0 eq.), (2-hydroxy-4-(trifluoromethyl)phenyl) boronic acid (150 mg, 0.73 mmol, 1.5 eq.), Na₂CO₃ (154 mg, 1.5 mmol, 3.0 eq.), and Pd(PPh₃)₄ (56 mg, 0.049 mmol, 0.1 eq.) were mixed. The tube was sealed and connected to a Schlenk line to undergo nitrogen/vacuum cycling three times, before the addition of 3 mL of 1,4-dioxane and 0.5 mL of DI water. The mixture was stirred at 110° C. (heating block) for 16 hours. After cooling to rt, the reaction was quenched with 10 mL of DI water. The mixture was extracted with EtOAc (10 mL×3). The combined organic extracts were dried over anhydrous Na₂SO₄, filtered, and concentrated under reduced pressure. The crude was further purified by preparative RP-HPLC (5-65% ACN in H₂O with 0.1% HCOOH in 30 min) to afford the title compound (R)-2-(3-((6-(2-hydroxy-4-(trifluoromethyl)phenyl)-4,5-dimethylpyridazin-3-yl)amino)piperidin-1-yl)ethyl 4-hydroxypiperidine-1-carboxylate (75) (88 mg, 34%) as a white solid after lyophilization of the combined product containing fractions. MS: mass calcd for C₂₆H₃₄F₃N₅O₄ 537.26, found 538.10, m/z=[M+H]⁺. ¹H NMR (400 MHz, Methanol-d₄): δ 7.39 (d, J=7.9 Hz, 1H), 7.24 (d, J=8.0 Hz, 1H), 7.17 (s, 1H), 4.53-4.44 (m, 1H), 4.42-4.31 (m, 2H), 3.87-3.70 (m, 3H), 3.68-3.52 (m, 1H), 3.28-3.16 (m, 3H), 3.15-3.01 (m, 2H), 2.92-2.72 (m, 2H), 2.20 (s, 3H), 2.12 (s, 3H), 2.10-1.99 (m, 2H), 1.92-1.65 (m, 4H), 1.46-1.28 (m, 2H) ppm. ¹⁹F NMR (376 MHz, Methanol-d₄): δ −64.29 ppm.

### Example 76: (R)-1-(2-(3-((6-(2-Hydroxy-4-(trifluoromethyl)phenyl)-4,5-dimethylpyridazin-3-yl)amino)piperidin-1-yl)ethyl)piperidin-4-ol (76)

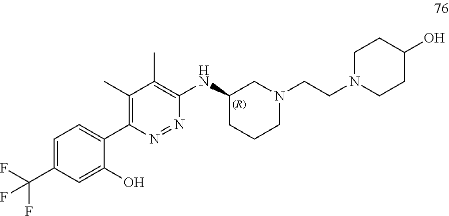

### Synthetic Route:

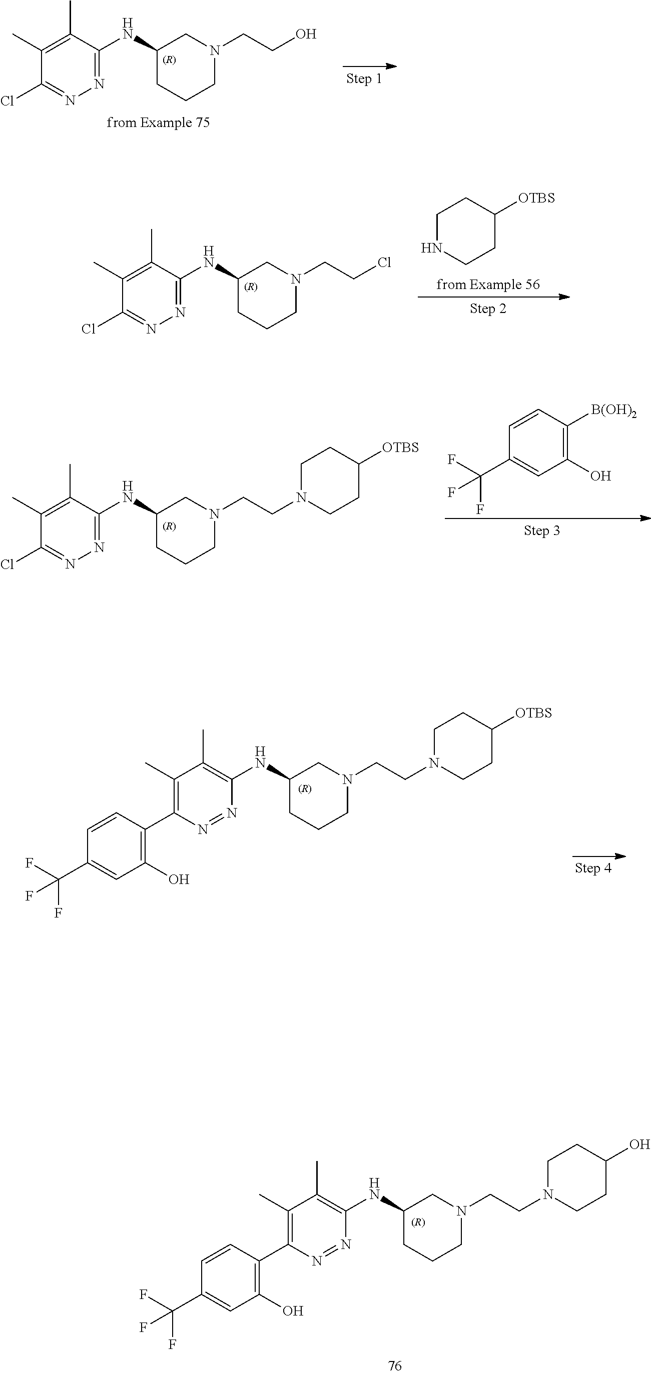

### Step 1: Synthesis of (R)-6-chloro-N-(1-(2-chloroethyl)piperidin-3-yl)-4,5-dimethylpyridazin-3-amine

In a single-necked flask, (R)-2-(3-((6-chloro-4,5-dimethylpyridazin-3-yl)amino)piperidin-1-yl)ethan-1-ol (prepared as disclosed in Example 75) (1.4 g, 4.9 mmol, 1.0 eq.) was dissolved in 20 mL of anhydrous THF followed by addition of TEA (1.5 g, 14.7 mmol, 3.0 eq.) and MsCl (1.7 g, 14.7 mmol, 3.0 eq.). The reaction mixture was stirred at rt for 1 h. The reaction mixture was concentrated by rotary evaporation under reduced pressure. The residue was purified by flash column chromatography (0-5% MeOH in DCM) to afford (R)-6-chloro-N-(1-(2-chloroethyl)piperidin-3-yl)-4,5-dimethylpyridazin-3-amine (600 mg, 34%) as a yellow oil after evaporation of the combined product containing fractions under reduced pressure using a rotary evaporator. LC/MS: mass calcd for C₁₃H₂₀Cl₂N₄ 302.11, found m/z=303.10 [M+H]⁺.

### Step 2: Synthesis of (R)—N-(1-(2-(4-((tert-butyldimethylsilyl)oxy) piperidin-1-yl)ethyl)piperidin-3-yl)-6-chloro-4,5-dimethylpyridazin-3-amine

In a single-necked flask, (R)-6-chloro-N-(1-(2-chloroethyl)piperidin-3-yl)-4,5-dimethylpyridazin-3-amine (600 mg, 2.0 mmol, 1.0 eq.) was dissolved in 20 mL of anhydrous ACN followed by addition of 4-((tert-butyldimethylsilyl)oxy)piperidine (prepared as disclosed in Example 56) (427 mg, 2.0 mmol, 1.0 eq.) and Cs₂CO₃ (1.3 g, 4.0 mmol, 2.0 eq.). The reaction mixture was stirred at 80° C. (heating block) for 16 h. Upon completion, the reaction mixture was allowed to cool to room temperature and was filtered through a Celite® cake. The filtrate was concentrated by rotary evaporation under reduced pressure. The residue was purified by flash column chromatography (0-5% MeOH in DCM) to afford (R)—N-(1-(2-(4-((tert-butyldimethylsilyl)oxy) piperidin-1-yl)ethyl)piperidin-3-yl)-6-chloro-4,5-dimethylpyridazin-3-amine (200 mg, 21%) as a yellow solid after evaporation of the combined product containing fractions under reduced pressure using a rotary evaporator.

¹H NMR (400 MHz, Methanol-d₄): δ 4.33-4.24 (m, 1H), 3.86-3.77 (m, 1H), 3.37 (s, 1H), 3.03 (d, J=10.7 Hz, 1H), 2.85-2.74 (m, 2H), 2.74-2.66 (m, 1H), 2.58 (s, 4H), 2.34 (s, 6H), 2.18 (s, 3H), 1.96-1.88 (m, 1H), 1.85-1.75 (m, 3H), 1.74-1.66 (m, 1H), 1.65-1.53 (m, 3H), 0.92 (s, 9H), 0.09 (s, 6H) ppm.

### Step 3: Synthesis of (R)-2-(6-((1-(2-(4-((tert-butyldimethylsilyl)oxy)piperidin-1-yl)ethyl)piperidin-3-yl)amino)-4,5-dimethylpyridazin-3-yl)-5-(trifluoromethyl)phenol

In a single necked flask, (R)—N-(1-(2-(4-((tert-butyldimethylsilyl)oxy) piperidin-1-yl)ethyl)piperidin-3-yl)-6-chloro-4,5-dimethylpyridazin-3-amine (130 mg, 0.3 mmol, 1.0 eq.) was dissolved in 5 mL of anhydrous 1,4-dioxane followed by the addition of (2-hydroxy-4-(trifluoromethyl)phenyl) boronic acid (84 mg, 0.4 mmol, 1.5 eq.), a 2 M aqueous Cs₂CO₃ solution (0.4 mL, 0.8 mmol, 3.0 eq.), and Pd(PPh₃)₂Cl₂ (44 mg, 0.06 mmol, 0.2 eq.). The reaction mixture was stirred at 110° C. (heating block) for 16 h under an atmosphere of nitrogen. Upon completion, the reaction mixture was allowed to cool to room temperature, was diluted with 40 mL of water and extracted with 3×10 mL EtOAc. The combined organic extracts were dried over anhydrous sodium sulfate. After filtration, the filtrate was concentrated by rotary evaporation under reduced pressure. The residue was purified by flash column chromatography (0-5% MeOH in DCM) to afford (R)-2-(6-((1-(2-(4-((tert-butyldimethylsilyl)oxy) piperidin-1-yl)ethyl)piperidin-3-yl)amino)-4,5-dimethylpyridazin-3-yl)-5-(trifluoromethyl)phenol (118 mg, 72%) as a yellow solid after evaporation of the combined product containing fractions under reduced pressure using a rotary evaporator. LC-MS: mass calcd, for C₃₁H₄₈F₃N₅O₂Si 607.35, found m/z=608.50 [M+H]⁺.

### Step 7: Synthesis of (R)-1-(2-(3-((6-(2-hydroxy-4-(trifluoromethyl)phenyl)-4,5-dimethylpyridazin-3-yl)amino)piperidin-1-yl)ethyl)piperidin-4-ol (76)

In a single necked flask, (R)-2-(6-((1-(2-(4-((tert-butyldimethylsilyl)oxy) piperidin-1-yl)ethyl)piperidin-3-yl)amino)-4,5-dimethylpyridazin-3-yl)-5-(trifluoromethyl)phenol (118 mg, 0.2 mmol, 1.0 eq.) was dissolved in 5 mL of THE followed by addition of tetrabutylammonium fluoride (126 mg, 0.40 mmol, 2.0 eq.). The reaction mixture was stirred at rt overnight. Upon completion, the reaction mixture was concentrated by rotary evaporation under reduced pressure. The residue was dissolved in 40 mL of water and extracted with 3×10 mL EtOAc. The combined organic extracts were washed with 3×30 mL of brine and dried over anhydrous sodium sulfate. After filtration, the filtrate was concentrated by rotary evaporation under reduced pressure. The residue was purified by preparative TLC. After isolation from the TLC plate, the product was freeze-dried to afford the target compound (R)-1-(2-(3-((6-(2-hydroxy-4-(trifluoromethyl)phenyl)-4,5-dimethylpyridazin-3-yl)amino)piperidin-1-yl)ethyl)piperidin-4-ol (76) (41 mg, 43%) as a white solid. LC/MS: mass calcd for C₂₅H₃₄F₃N₅O₂ 493.27, found m/z=494.30, [M+H]⁺.

¹H NMR (400 MHz, Methanol-d₄): δ 7.37 (d, J=7.9 Hz, 1H), 7.21 (d, J=7.8 Hz, 1H), 7.16 (s, 1H), 4.46-4.25 (m, 1H), 3.74-3.56 (m, 1H), 3.15-3.04 (m, 1H), 2.95-2.86 (m, 2H), 2.74-2.66 (m, 1H), 2.65-2.55 (m, 4H), 2.41-2.20 (m, 4H), 2.16 (s, 3H), 2.09 (s, 3H), 1.98-1.91 (m, 1H), 1.90-1.84 (m, 2H), 1.83-1.76 (m, 1H), 1.70-1.54 (m, 4H) ppm. ¹⁹F NMR (376 MHz, Methanol-d₄): δ −64.24 ppm.

### Example 77: (R)-1-(3-Aminoazetidin-1-yl)-2-(3-((6-(2-hydroxy-4-(trifluoromethyl)phenyl)-5-methylpyridazin-3-yl)amino)piperidin-1-yl)ethan-1-one (77)

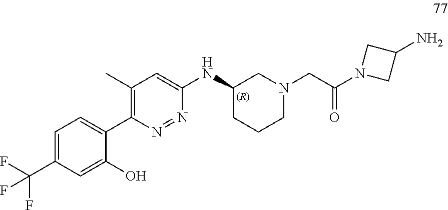

### Synthetic Route:

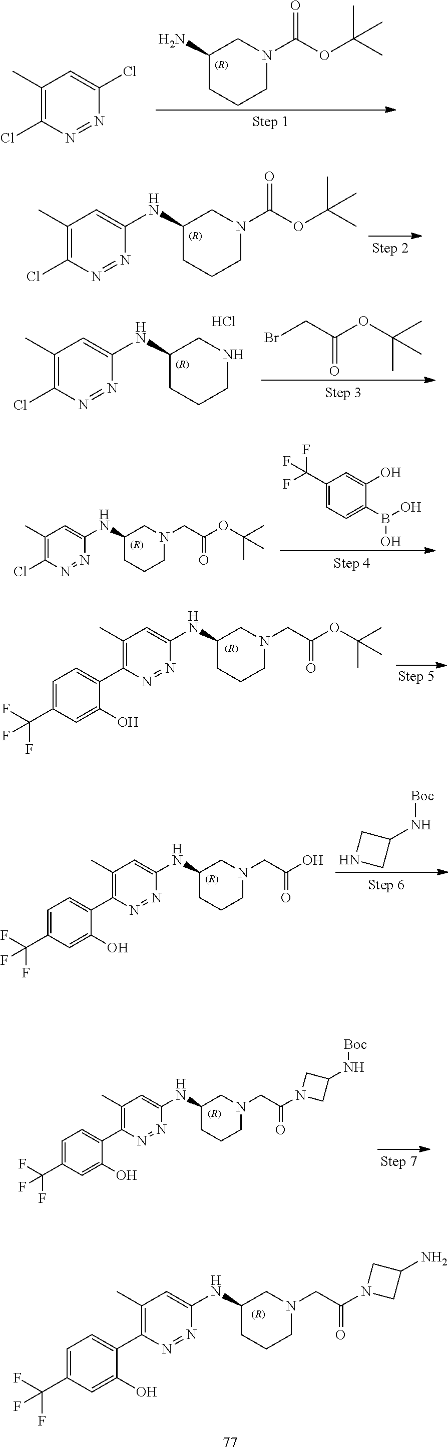

### Step 1: Synthesis of tert-butyl (R)-3-((6-chloro-5-methylpyridazin-3-yl)amino)piperidine-1-carboxylate

A brown solution of 3,6-dichloro-4-methylpyridazine (5.00 g, 30.7 mmol) and tert-butyl (3R)-3-aminopiperidine-1-carboxylate (6.76 g, 33.7 mmol, 1.0 eq.) and DIEA (16.0 mL, 11.9 g, 92.0 mmol, ~2.7 eq.) in NMP (25 mL) was stirred for overnight at 150° C. After cooling to rt, EtOAc (100 mL) and water (100 mL) were added to the reaction mixture. After separation of the phases, the aqueous phase was extracted with EtOAc (2×50 mL). The combined organic phases were washed with brine (2×200 mL), dried over anhydrous Na₂SO₄, filtered, and the filtrate was concentrated under reduced pressure using a rotary evaporator. After concentration, the suspension was subjected to silica gel column chromatography purification eluting with PE/EtOAc gradients (0~50%) to afford tert-butyl (R)-3-((6-chloro-5-methylpyridazin-3-yl)amino)piperidine-1-carboxylate as a yellow oil (1.3 g, 13.0% yield) after evaporation of the combined product containing fractions under reduced pressure using a rotary evaporator. LC/MS: mass calcd for C₁₅H₂₃ClN₄O₂: 326.15, found: m/z=327.15 [M+H]⁺.

### Step 2: Synthesis of (R)-6-chloro-5-methyl-N-(piperidin-3-yl)pyridazin-3-amine hydrochloride

To a solution of tert-butyl (R)-3-((6-chloro-5-methylpyridazin-3-yl)amino)piperidine-1-carboxylate (680 mg, 2.08 mmol, 1.0 eq.) in DCM (3 mL) was added hydrogen chloride (HCl) (3 mL, 4 M in 1,4-dioxane, 12 mmol, ~6 eq.) at 0° C. (ice bath). The resulting solution was stirred at rt for 2 h. The reaction mixture was concentrated under reduced pressure using a rotary evaporator to afford (R)-6-chloro-5-methyl-N-(piperidin-3-yl)pyridazin-3-amine hydrochloride as a colorless solid (550 mg, 90.4% yield). LC/MS: mass calcd for C₁₀H₁₅ClN₄: 226.10, found: m/z=227.00 [M+H]⁺.

### Step 3: Synthesis of tert-butyl (R)-2-(3-((6-chloro-5-methylpyridazin-3-yl)amino)piperidin-1-yl)acetate

A brown solution of (R)-6-chloro-5-methyl-N-(piperidin-3-yl)pyridazin-3-amine hydrochloride (600 mg, 2.65 mmol, 1.0 eq.), tert-butyl 2-bromoacetate (391 μL, 516 mg, 2.65 mmol, 1.0 eq.) and DIEA (1.84 mL, 1.37 g, 10.6 mmol, 4.0 eq.) in ACN (7 mL) was stirred at 60° C. for 1 h. After cooling to room temperature, the volatiles were removed under reduced pressure using a rotary evaporator. The residue was subjected to silica gel column purification eluting with PE/EtOAc gradients (0~100%) to afford tert-butyl (R)-2-(3-((6-chloro-5-methylpyridazin-3-yl)amino)piperidin-1-yl)acetate as a yellow solid (650 mg, 68.7% yield) after evaporation of the combined product containing fractions under reduced pressure using a rotary evaporator. LC/MS: mass calcd for C₁₆H₂₅ClN₄O₂: 340.17, found: m/z=341.10 [M+H]⁺.

### Step 4: Synthesis of tert-butyl (R)-2-(3-((6-(2-hydroxy-4-(trifluoromethyl)phenyl)-5-methylpyridazin-3-yl)amino)piperidin-1-yl)acetate

To a solution of tert-butyl (R)-2-(3-((6-chloro-5-methylpyridazin-3-yl)amino)piperidin-1-yl)acetate (1.00 g, 2.93 mmol, 1.0 eq.), (2-hydroxy-4-(trifluoromethyl)phenyl) boronic acid (0.91 g, 4.40 mmol, 1.5 eq.) in a 21 mL of a mixture of 1,4-dioxane/H₂O (2:1, v/v) was added NaHCO₃ (0.74 g, 8.80 mmol, 3.0 eq.) and Pd(PPh₃)₄ (0.1 g, 0.09 mmol, 0.03 eq.). The reaction mixture was stirred in a sealed tube under an atmosphere of N₂ at 150° C. for 1 h. After the reaction mixture cooled down to rt, the reaction was quenched with an aqueous solution of NaCl (20 mL) and further diluted with water (20 mL). The aqueous phase was extracted with EtOAc (3×30 mL). The combined organic phases were washed with brine (2×80 mL), dried over Na₂SO₄, filtered, and the filtrate was concentrated under reduced pressure using a rotary evaporator. This residue was subjected to silica gel column chromatography purification to afford tert-butyl (R)-2-(3-((6-(2-hydroxy-4-(trifluoromethyl)phenyl)-5-methylpyridazin-3-yl)amino)piperidin-1-yl)acetate as an off-white solid (1.1 g, 75.5% yield) after evaporation of the combined product containing fractions under reduced pressure using a rotary evaporator. LC/MS: mass calcd for C₂₃H₂₉F₃N₄O₃: 466.22, found: m/z=467.18 [M+H]⁺.

### Step 5: Synthesis of (R)-2-(3-((6-(2-hydroxy-4-(trifluoromethyl)phenyl)-5-methylpyridazin-3-yl)amino)piperidin-1-yl)acetic Acid

To a yellow solution of tert-butyl (R)-2-(3-((6-(2-hydroxy-4-(trifluoromethyl)phenyl)-5-methylpyridazin-3-yl)amino)piperidin-1-yl)acetate (1.10 g, 2.54 mmol, 1.0 eq.) in DCM (4 mL), HCl (8 mL, 4 M in 1,4-dioxane, 32 mmol, ~12.6 eq.) was added resulting in a suspension which was stirred for 4 h at rt. The solvents were removed under reduced pressure using a rotary evaporator to afford (R)-2-(3-((6-(2-hydroxy-4-(trifluoromethyl)phenyl)-5-methylpyridazin-3-yl)amino)piperidin-1-yl)acetic acid as a yellow solid (880 mg, 77.3% yield) which was used directly without further isolation, purification, and characterization. LC/MS: mass calcd for C₁₉H₂₁F₃N₄O₃: 410.16, found: m/z=411.15 [M+H]⁺.

### Step 6: Synthesis of tert-butyl (R)-(1-(2-(3-((6-(2-hydroxy-4-(trifluoromethyl)phenyl)-5-methylpyridazin-3-yl)amino)piperidin-1-yl)acetyl)azetidin-3-yl)carbamate

A yellow solution of (R)-2-(3-((6-(2-hydroxy-4-(trifluoromethyl)phenyl)-5-methylpyridazin-3-yl)amino)piperidin-1-yl)acetic acid (100 mg, 0.244 mmol), tert-butyl azetidin-3-yl carbamate (50 mg, 0.29 mmol, 1.2 eq.), EDCI (70 mg, 0.37 mmol, 1.5 eq.), HOBt (49 mg, 0.37 mmol, 1.5 eq.) and DIEA (127 μL, 94 mg, 0.73 mmol, 3.0 eq.) in DMF (2 mL) was stirred at rt for 1 h. To this solution, EtOAc (10 mL) and water (10 mL) were added. After separation of phases, the aqueous phase was extracted with EtOAc (2×10 mL). The combined organic phases were washed with brine (3×30 mL), dried with anhydrous Na₂SO₄, filtered, and the filtrate was concentrated under reduced pressure using a rotary evaporator. The residue was purified by silica gel column chromatography eluting with eluting with PE/EtOAc gradients (0~40%) to afford tert-butyl (R)-(1-(2-(3-((6-(2-hydroxy-4-(trifluoromethyl)phenyl)-5-methylpyridazin-3-yl)amino)piperidin-1-yl)acetyl)azetidin-3-yl)carbamate as a yellow solid (110 mg, 46.3% yield) after evaporation of the combined product containing fractions under reduced pressure using a rotary evaporator. LC/MS: mass calcd for C₂₇H₃₅F₃N₆O₄: 564.27, found: 565.00 [M+H]⁺.

### Step 7: Synthesis of (R)-1-(3-aminoazetidin-1-yl)-2-(3-((6-(2-hydroxy-4-(trifluoromethyl)phenyl)-5-methylpyridazin-3-yl)amino)piperidin-1-yl)ethan-1-one (77)

To a yellow solution of tert-butyl (R)-(1-(2-(3-((6-(2-hydroxy-4-(trifluoromethyl)phenyl)-5-methylpyridazin-3-yl)amino)piperidin-1-yl)acetyl)azetidin-3-yl)carbamate (110 mg, 0.195 mmol, 1.0 eq.) in DCM (2 mL), HCl (1 mL, 4 M in 1,4-dioxane, 4 mmol, ~20 eq.) was added affording a suspension which was stirred at rt for 4 h. The solvents were removed using a rotary evaporator under reduced pressure affording a yellow solid. The yellow solid was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 50% B in 7 min, 50% B; Wavelength: 254 nm; RT1: 5.43 min. After lyophilization of the combined product containing fractions, the target compound (R)-1-(3-aminoazetidin-1-yl)-2-(3-((6-(2-hydroxy-4-(trifluoromethyl)phenyl)-5-methylpyridazin-3-yl)amino)piperidin-1-yl)ethan-1-one (77) was obtained as an off-white solid (12.0 mg, 13.2% yield). LC/MS: mass calcd for C₂₂H₂₇F₃N₆O₂: 464.21, found: 465.20 [M+H]⁺. ¹H NMR (400 MHz, Methanol-d₄): δ 7.63-7.53 (m, 1H), 7.30-7.11 (m, 2H), 6.91-6.68 (m, 1H), 4.69-4.43 (m, 1H), 4.32-3.98 (m, 3H), 3.95-3.79 (m, 1H), 3.76-3.61 (m, 1H), 3.23-3.01 (m, 2H), 2.99-2.81 (m, 1H), 2.65-2.25 (m, 1H), 2.46-2.25 (m, 2H), 2.25-2.08 (m, 3H), 1.91-1.80 (m, 2H), 1.76-1.46 (m, 2H) ppm. ¹⁹F NMR (376 MHz, Methanol-d₄): δ −64.27 ppm.

### Example 78: (R)-2-(6-((1-(2-Aminoethyl)piperidin-3-yl)amino)-4-methylpyridazin-3-yl)-5-(trifluoromethyl)phenol (78)

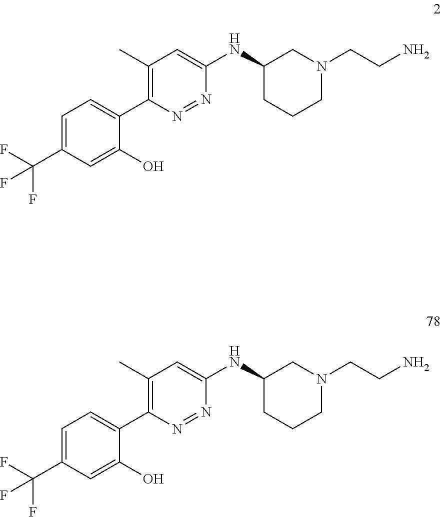 [SMILES: Cc1cc(N[C@@H]2CCCN(CCN)C2)nnc1-c1ccc(C(F)(F)F)cc1O ; Cc1cc(N[C@@H]2CCCN(CCN)C2)nnc1-c1ccc(C(F)(F)F)cc1O]

### Synthetic Route:

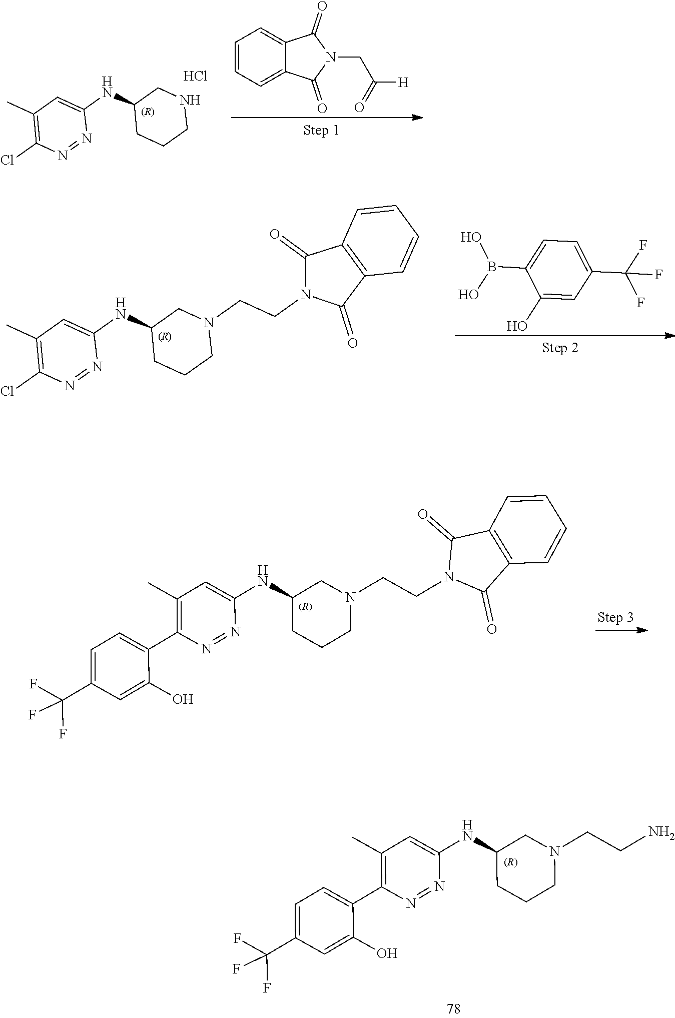

### Step 1: Synthesis of (R)-2-(2-(3-((6-chloro-5-methylpyridazin-3-yl)amino)piperidin-1-yl)ethyl) isoindoline-1,3-dione

A colorless solution of (R)-6-chloro-5-methyl-N-(piperidin-3-yl)pyridazin-3-amine hydrochloride (200 mg, 0.8 mmol, 1.0 eq.) and 2-(1,3-dioxoisoindolin-2-yl)acetaldehyde (144 mg, 0.8 mmol, 1.0 eq.) in MeOH (5 mL) was stirred at rt for 0.5 h. To this solution, solid NaBH₃CN (62 mg, 0.9 mmol, 1.1 eq.) was added in small portions. The obtained solution was stirred overnight at rt The resulting reaction mixture was concentrated under reduced pressure using a rotary evaporator. The residue was purified by silica gel column chromatography eluting with PE/EtOAc gradients (0~50%) to afford (R)-2-(2-(3-((6-chloro-5-methylpyridazin-3-yl)amino)piperidin-1-yl)ethyl) isoindoline-1,3-dione as a white solid (230 mg, 75.6% yield) after evaporation of the combined product containing fractions under reduced pressure using a rotary evaporator. LC/MS (ESI): mass calcd for C₂₀H₂₂ClN₅O₂: 399.15, found: m/z=400.15 [M+H]⁺.

### Step 2: Synthesis of (R)-2-(2-(3-((6-(2-hydroxy-4-(trifluoromethyl)phenyl)-5-methylpyridazin-3-yl)amino)piperidin-1-yl)ethyl) isoindoline-1,3-dione

A brown suspension of (R)-2-(2-(3-((6-chloro-5-methylpyridazin-3-yl)amino)piperidin-1-yl)ethyl) isoindoline-1,3-dione (200 mg, 0.5 mmol, 1.0 eq.), (2-hydroxy-4-(trifluoromethyl)phenyl) boronic acid (175 mg, 0.9 mmol, 1.7 eq.), Pd(PPh₃)₄ (18.0 mg, 0.02 mmol, 0.03 eq.) and NaHCO₃ (84.0 mg, 1.0 mmol, 2.0 eq.) in 3 mL of a mixture of 1,4-dioxane and H₂O (2:1, v/v) in a sealed tube was stirred under an atmosphere of N₂ at 150° C. for 1 h. After the reaction mixture cooled to rt, EtOAc (20 mL) and water (20 mL) were added to the suspension. After separation of the phases, the aqueous phase was extracted with EtOAc (2×10 mL). The combined organic phases were washed with brine (3×30 mL), dried over anhydrous Na₂SO₄, filtered, and the filtrate was concentrated under reduced pressure using a rotary evaporator. The residue was purified by silica gel column chromatography eluting with DCM/MeOH gradients (0~20%) to yield (R)-2-(2-(3-((6-(2-hydroxy-4-(trifluoromethyl)phenyl)-5-methylpyridazin-3-yl)amino)piperidin-1-yl)ethyl) isoindoline-1,3-dione as a light brown oil (230 mg, 87.5% yield) after evaporation of the combined product containing fractions under reduced pressure using a rotary evaporator. LC/MS (ESI): mass calcd for C₂₇H₂₆F₃N₅O₃: 525.20, found: m/z=526.10 [M+H]⁺.

### Step 3: Synthesis of (R)-2-(6-((1-(2-aminoethyl)piperidin-3-yl)amino)-4-methylpyridazin-3-yl)-5-(trifluoromethyl)phenol (78)

A colorless solution of (R)-2-(2-(3-((6-(2-hydroxy-4-(trifluoromethyl)phenyl)-5-methylpyridazin-3-yl)amino)piperidin-1-yl)ethyl) isoindoline-1,3-dione (180 mg, 0.3 mmol, 1.0 eq.) and hydrazine hydrate (NH₂NH₂·H₂O) (2 mL, 80%) in MeOH (8 mL) was stirred at 80° C. for 4 h (oil bath). After cooling to rt, the resulting reaction mixture was concentrated under reduced pressure using a rotary evaporator. The crude product was purified by preparative RP-HPLC using the following conditions: Column: Xselect CSH C18 OBD Column 30*150 mm 5 μm; Mobile Phase A: Water (10 mmol/L FA aq.), Mobile Phase B: ACN; Flow rate: 60 mL/min; Gradient: 3% B to 16% B in 7 min, 16% B; Wavelength: 254 nm; RT: 5.40 min. The target compound (R)-2-(6-((1-(2-aminoethyl)piperidin-3-yl)amino)-4-methylpyridazin-3-yl)-5-(trifluoromethyl)phenol (78) format salt obtained as an off-white solid (30 mg, 19.3% yield) after lyophilization of the combined product containing fractions. LC/MS (ESI): mass calcd for C₁₉H₂₄F₃N₅O: 395.19, found: m/z=396.20 [M+H]⁺. ¹H NMR (400 MHz, Methanol-d₄): δ 7.44-7.37 (m, 1H), 7.26-7.14 (m, 1H), 7.20 (s, 1H), 6.79 (s, 1H), 4.11-4.00 (m, 1H), 3.27-3.15 (m, 1H), 3.14-2.99 (m, 2H), 2.87-2.67 (m, 2H), 2.62-2.52 (m, 1H), 2.38-2.23 (m, 1H), 2.16 (s, 3H), 2.06-1.90 (m, 2H), 1.88-1.69 (m, 2H), 1.53-1.40 (m, 1H) ppm. ¹⁹F NMR (376 MHz, Methanol-d₄): δ −64.29 ppm.

### Example 79: (R)—N-(2-Aminoethyl)-2-(3-((6-(2-hydroxy-4-(trifluoromethyl)phenyl)-5-methylpyridazin-3-yl)amino)piperidin-1-yl)-N-methylacetamide (79)

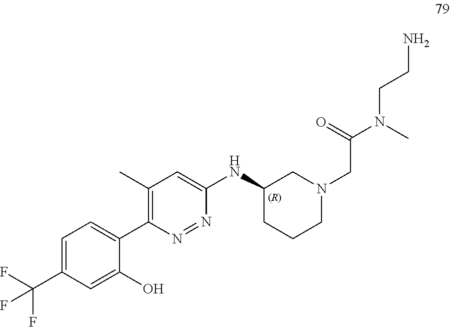

### Synthetic Route:

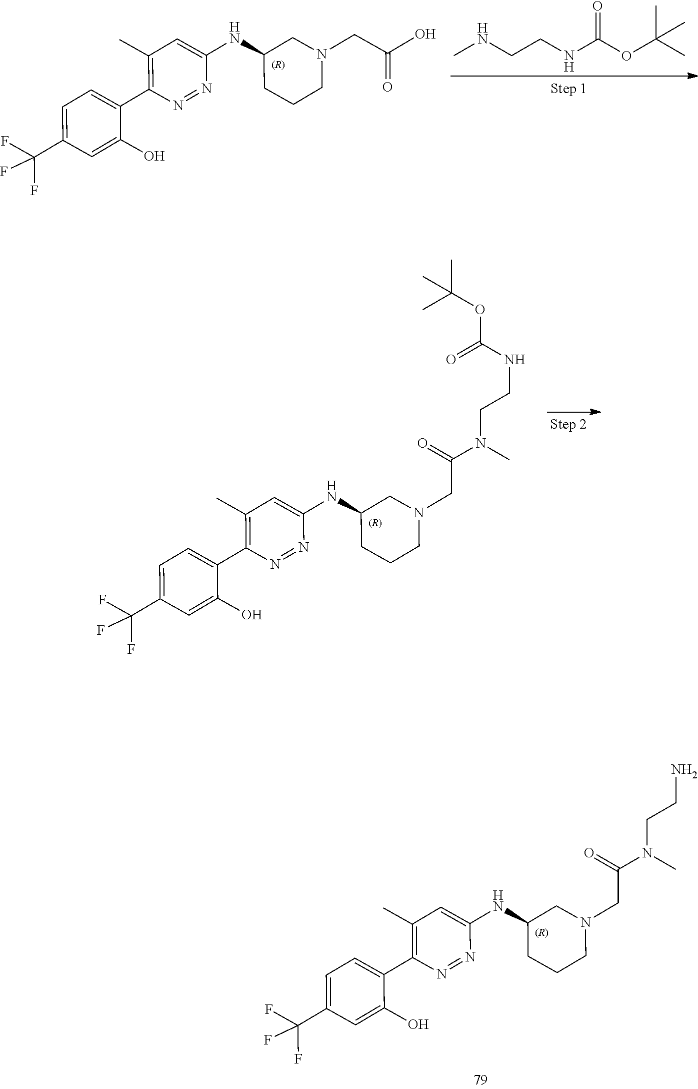

### Step 1: Synthesis of tert-butyl (R)-(2-(2-(3-((6-(2-hydroxy-4-(trifluoromethyl)phenyl)-5-methylpyridazin-3-yl)amino)piperidin-1-yl)-N-methylacetamido)ethyl)carbamate

To a solution of (R)-2-(3-((6-(2-hydroxy-4-(trifluoromethyl)phenyl)-5-methylpyridazin-3-yl)amino)piperidin-1-yl)acetic acid (100 mg, 0.2 mmol, 1.0 eq.) in DMF (2 mL) were added tert-butyl(2-(methylamino)ethyl)carbamate (43 mg, 0.2 mmol, 1.0 eq.), HOBt (50 mg, 0.3 mmol, 1.5 eq.) and EDCI (71 mg, 0.3 mmol, 1.5 eq.). The resulting solution was stirred at rt for 2 h. The reaction mixture was diluted with EtOAc (10 mL) and brine (10 mL). After separation of organic phase, the aqueous phase was extracted with EtOAc (2×10 mL). The combined organic phases were washed with brine (20 mL), dried over anhydrous Na₂SO₄, filtered, the filtrate was concentrated under reduced pressure using a rotary evaporator to afford tert-butyl (R)-(2-(2-(3-((6-(2-hydroxy-4-(trifluoromethyl)phenyl)-5-methylpyridazin-3-yl)amino)piperidin-1-yl)-N-methylacetamido)ethyl)carbamate as a brown oil (100 mg, crude). The crude product was used directly in the next step without further purification, isolation, and characterization. LC/MS (ESI): mass calcd for: C₂₇H₃₇F₃N₆O₄: 566.28, found: m/z=567.30 [M+H]⁺.

### Step 2: Synthesis of (R)—N-(2-aminoethyl)-2-(3-((6-(2-hydroxy-4-(trifluoromethyl)phenyl)-5-methylpyridazin-3-yl)amino)piperidin-1-yl)-N-methylacetamide (79)

To a solution of tert-butyl (R)-(2-(2-(3-((6-(2-hydroxy-4-(trifluoromethyl)phenyl)-5-methylpyridazin-3-yl)amino)piperidin-1-yl)-N-methylacetamido)ethyl)carbamate (100 mg, 0.2 mmol, 1.0 eq.) in DCM (1 mL) was added HCl (4 M in dioxane) (1 mL, 4 mmol, 20 eq.) at 0° C. (ice bath). The resulting solution was stirred at rt for 2 h. The reaction mixture was concentrated under reduced pressure using a rotary evaporator. The crude product was purified by preparative RP-HPLC using the following conditions: Column: XBridge Prep OBD C18 Column, 30*150 mm, 5 μm; Mobile Phase A: Water (10 mmol/L NH₄HCO₃; 0.1% NH₃), Mobile Phase B: ACN; Flow rate: 60 mL/min; Gradient: 25% B to 55% B in 7 min; Wavelength: 254 nm; RT: 6 min. The target compound (R)—N-(2-aminoethyl)-2-(3-((6-(2-hydroxy-4-(trifluoromethyl)phenyl)-5-methylpyridazin-3-yl)amino)piperidin-1-yl)-N-methylacetamide (79) was obtained as a white solid (12 mg, 13.9% yield) after lyophilization of the combined product containing fractions. LC/MS (ESI): mass calcd for C₂₂H₂₉F₃N₆O₂: 466.23, found: m/z=467.25 [M+H]⁺. ¹H NMR (400 MHz, DMSO-d₆): δ 7.39-7.36 (m, 1H), 7.23-7.18 (m, 2H), 6.68 (s, 1H), 4.02-3.97 (m, 1H), 3.51-3.50 (m, 1H), 3.40-3.37 (m, 1H), 3.33-3.22 (m, 1H), 3.19-3.10 (m, 2H), 3.04-3.00 (m, 1H), 2.98-2.88 (m, 1H), 2.77-2.73 (m, 2H), 2.68-2.56 (m, 2H), 2.24-2.06 (m, 2H), 2.01 (s, 3H), 1.91-1.81 (m, 1H), 1.78-1.66 (m, 1H), 1.62-1.45 (m, 1H), 1.40-1.23 (m, 1H) ppm. ¹⁹F NMR (376 MHz, DMSO-d₆): δ −61.14 ppm.

### Example 80: (R)-2-(4-((1-(Azetidin-3-yl)piperidin-3-yl)amino)-6,7-dihydro-5H-cyclopenta[d]pyridazin-1-yl)-5-(trifluoromethyl)phenol (80)

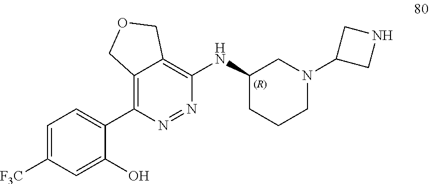

### Synthetic Route:

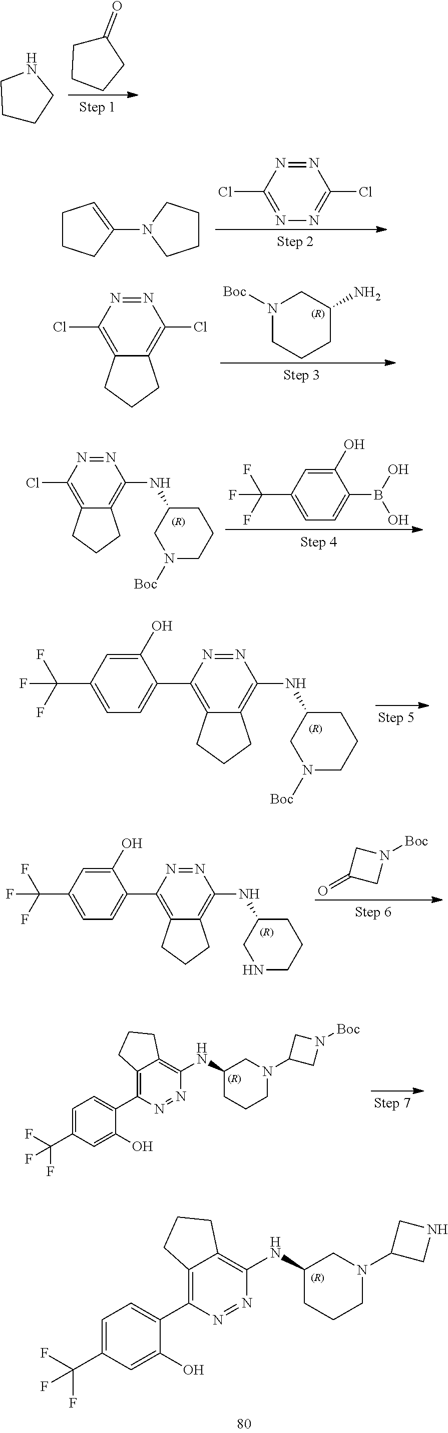

### Step 1: Synthesis of 1-(cyclopent-1-en-1-yl)pyrrolidine

To a light yellow solution of pyrrolidine (10.0 g, 141 mmol, 1.0 eq.) in toluene (50 mL) were added cyclopentanone (11.8 g, 12.4 mL, 141 mmol, 1.0 eq.) and TsOH (0.1 g, 0.5 mmol, 0.004 eq.). After stirring for 16 h under reflux at 130° C. (oil bath), the reaction mixture was cooled to rt, and the residue was concentrated under reduced pressure using a rotary evaporator yielding 1-(cyclopent-1-en-1-yl)pyrrolidine (11 g, 57% yield) as brown oil. The residue was used directly in the next step without further isolation, purification, or characterization.

### Step 2: Synthesis of 1,4-dichloro-6,7-dihydro-5H-cyclopenta[d]pyridazine

To a red solution of dichloro-1,2,4,5-tetrazine (4.4 g, 29 mmol, 1.0 eq.) in DCM (150 mL) was added crude 1-(cyclopent-1-en-1-yl)pyrrolidine (8 g, 58 mmol, 2.0 eq.) at 0° C. (ice bath). After stirring of the reaction mixture for 15 min at 0° C., the residue was concentrated under reduced pressure using a rotary evaporator at 0° C. The residue was purified by silica gel column chromatography (0~45% diethyl ether/PE) yielding 1,4-dichloro-6,7-dihydro-5H-cyclopenta[d]pyridazine (2.6 g, 23.6% yield) as light yellow solid after evaporation of the combined product containing fractions under reduced pressure using a rotary evaporator. LC/MS (ESI): mass calcd for C₇H₆Cl₂N₂: 187.99, found: m/z=189.10 [M+H]⁺.

### Step 3: Synthesis of tert-butyl (R)-3-((4-chloro-6,7-dihydro-5H-cyclopenta[d]pyridazin-1-yl)amino)piperidine-1-carboxylate

To a light yellow of 1,4-dichloro-6,7-dihydro-5H-cyclopenta[d]pyridazine (0.9 g, 4.7 mmol, 1.0 eq.) in NMP (10 mL) were added tert-butyl (R)-3-aminopiperidine-1-carboxylate hydrochloride (0.6 g, 5.7 mmol, 1.2 eq.) and DIEA (1.2 g, 1.62 mL, 9.5 mmol, 2 eq.). After stirring of the reaction mixture for 12 h at 150° C. (oil bath), the reaction mixture was cooled to rt, the residue was extracted with EA (3×50 mL). The combined organic phases were washed with water (3×50 mL), dried over anhydrous Na₂SO₄, filtered, and the filtrated concentrated under reduced pressure using a rotary evaporator. The residue was purified by silica gel column chromatography (0~45% EtOAc/PE) yielding tert-butyl (R)-3-((4-chloro-6,7-dihydro-5H-cyclopenta[d]pyridazin-1-yl)amino)piperidine-1-carboxylate (0.5 g, 32.7% yield) as light yellow solid after evaporation of the combined product containing fractions using a rotary evaporator. LC/MS (ESI): mass calcd for C₁₇H₂₅ClN₄O₂: 352.17, found: m/z=353.20 [M+H]⁺.

### Step 4: Synthesis of tert-butyl (R)-3-((4-(2-hydroxy-4-(trifluoromethyl)phenyl)-6,7-dihydro-5H-cyclopenta[d]pyridazin-1-yl)amino)piperidine-1-carboxylate

To a solution of tert-butyl (R)-3-((4-chloro-6,7-dihydro-5H-cyclopenta[d]pyridazin-1-yl)amino)piperidine-1-carboxylate (250 mg, 0.7 mmol, 1.0 eq.) and (2-hydroxy-4-(trifluoromethyl)phenyl) boronic acid (175 mg, 0.9 mmol, 1.2 eq.) in a mixture of 1,4-dioxane (3 mL) and water (0.8 mL) were added Pd(PPh₃)₄ (41 mg, 0.04 mmol, 0.05 eq.) and solid NaHCO₃ (119 mg, 1.4 mmol, 2 eq.). After stirring for 5 h at 100° C. (oil bath) under an atmosphere of N₂, the reaction mixture was cooled to rt and concentrated under reduced pressure using a rotary evaporator. The residue was purified by silica gel column chromatography (0~50% EtOAc/PE) yielding tert-butyl (R)-3-((4-(2-hydroxy-4-(trifluoromethyl)phenyl)-6,7-dihydro-5H-cyclopenta[d]pyridazin-1-yl)amino)piperidine-1-carboxylate (246 mg, 72.6% yield) as light yellow solid after evaporation of the combined product containing fractions under reduced pressure using a rotary evaporator. LC/MS (ESI): mass calcd for C₂₄H₂₉F₃N₄O₃: 478.22, found: m/z=479.25 [M+H]⁺.

### Step 5: Synthesis of (R)-2-(4-(piperidin-3-ylamino)-6,7-dihydro-5H-cyclopenta[d]pyridazin-1-yl)-5-(trifluoromethyl)phenol

To a light yellow solution of tert-butyl (R)-3-((4-(2-hydroxy-4-(trifluoromethyl)phenyl)-6,7-dihydro-5H-cyclopenta[d]pyridazin-1-yl)amino)piperidine-1-carboxylate (200 mg, 0.4 mmol, 1.0 eq.) in DCM (5 mL) was added TFA (1 mL). After stirring the reaction mixture for 2 h at rt, the suspension was concentrated under reduced pressure using a rotary evaporator. The residue was purified by reversed flash chromatography using the following conditions: column, C18 silica gel; mobile phase, ACN in water, 10% to 60% gradient in 20 min; Wavelength: 254 nm, yielding (R)-2-(4-(piperidin-3-ylamino)-6,7-dihydro-5H-cyclopenta[d]pyridazin-1-yl)-5-(trifluoromethyl)phenol (71 mg, 44.9% yield) as light yellow solid after lyophilization of the combined product containing fractions. LC/MS (ESI): mass calcd for C₁₉H₂₁F₃N₄O: 378.17, found: m/z=379.20 [M+H]⁺.

### Step 6: Synthesis of tert-butyl (R)-3-(3-((4-(2-hydroxy-4-(trifluoromethyl)phenyl)-6,7-dihydro-5H-cyclopenta[d]pyridazin-1-yl)amino)piperidin-1-yl)azetidine-1-carboxylate

To a light yellow solution of (R)-2-(4-(piperidin-3-ylamino)-6,7-dihydro-5H-cyclopenta[d]pyridazin-1-yl)-5-(trifluoromethyl)phenol (71 mg, 0.2 mmol, 1 eq.) in MeOH (2 mL) were added tert-butyl 3-oxoazetidine-1-carboxylate (39 mg, 0.2 mmol, 1.2 eq.) and NaBH₃CN (35 mg, 0.6 mmol, 3 eq.). After stirring for 2 h at rt, the suspension was concentrated under reduced pressure using a rotary evaporator. The residue was purified by silica gel column chromatography using a 0~60% EtOAc/PE gradient to afford tert-butyl (R)-3-(3-((4-(2-hydroxy-4-(trifluoromethyl)phenyl)-6,7-dihydro-5H-cyclopenta[d]pyridazin-1-yl)amino)piperidin-1-yl)azetidine-1-carboxylate (80 mg, 79.9% yield) as a light yellow solid after evaporation of the product containing fractions under reduced pressure using a rotary evaporator. LC/MS (ESI): mass calcd for C₂₇H₃₄F₃N₅O₃: 533.26, found: m/z=534.35 [M+H]⁺.

### Step 7: Synthesis of (R)-2-(4-((1-(azetidin-3-yl)piperidin-3-yl)amino)-6,7-dihydro-5H-cyclopenta[d]pyridazin-1-yl)-5-(trifluoromethyl)phenol (80)

To a light yellow solution of tert-butyl (R)-3-(3-((4-(2-hydroxy-4-(trifluoromethyl)phenyl)-6,7-dihydro-5H-cyclopenta[d]pyridazin-1-yl)amino)piperidin-1-yl)azetidine-1-carboxylate (80 mg, 0.2 mmol, 1 eq.) in DCM (5 mL) was added TFA (1 mL). After stirring the reaction mixture for 2 h at rt, the suspension was concentrated under reduced pressure using a rotary evaporator. The residue was purified by reversed phase flash chromatography using the following conditions: Column: XBridge Prep Phenyl OBD Column, 19*100 mm, 5 μm; Mobile Phase A: Water (10 mmol/L NH₄HCO₃; 0.1% NH₃), Mobile Phase B: ACN; Flow rate: 60 mL/min; Gradient: 5% B to 95% B in 7 min, 95% B; Wavelength: 254 nm; RT: 6 min. The target compound (R)-2-(4-((1-(azetidin-3-yl)piperidin-3-yl)amino)-6,7-dihydro-5H-cyclopenta[d]pyridazin-1-yl)-5-(trifluoromethyl)phenol (80) (5.0 mg, 7.5% yield) was obtained as a light yellow solid after lyophilization of the combined product containing fractions. LC/MS (ESI): mass calcd for C₂₂H₂₆F₃N₅O: 433.21, found: m/z=434.20 [M+H]⁺. ¹H NMR (300 MHz, Methanol-d₄): δ 7.62-7.56 (m, 1H), 7.31-7.26 (m, 2H), 4.19-4.11 (m, 5H), 3.73-3.53 (m, 1H), 3.25-3.10 (m, 1H), 3.07-2.99 (m, 4H), 2.90-2.81 (m, 1H), 2.33-2.23 (m, 4H), 2.13-2.08 (m, 1H), 2.03-1.99 (m, 1H), 1.80-1.61 (m, 2H) ppm. ¹⁹F NMR (282 MHz, Methanol-d₄): δ −64.44 ppm.

### Example 81: (R)-2-(4-((1-(Azetidin-3-yl)piperidin-3-yl)amino)-6,7-dihydro-5H-cyclopenta[d]pyridazin-1-yl)-5-fluorophenol (81)

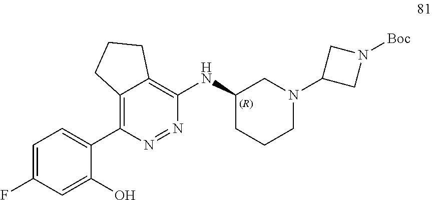

### Synthetic Route:

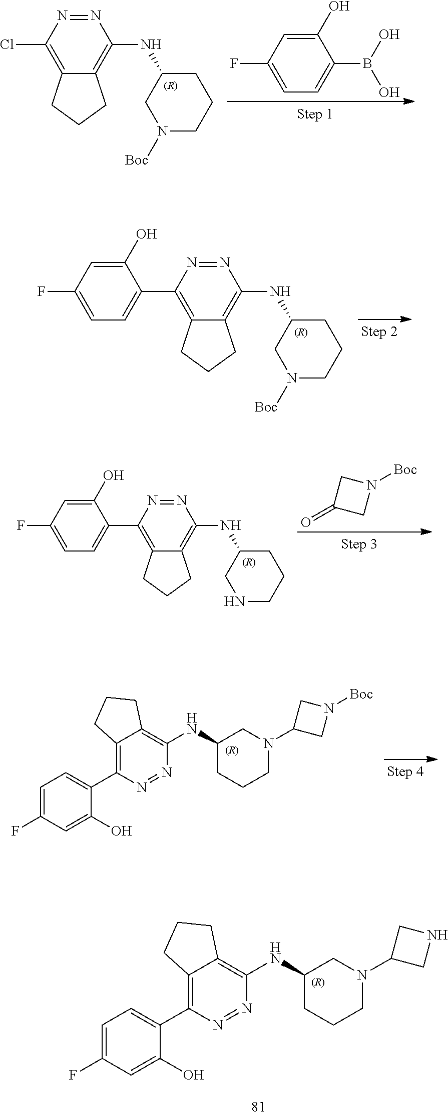

### Step 1: Synthesis of tert-butyl (R)-3-((4-(4-fluoro-2-hydroxyphenyl)-6,7-dihydro-5H-cyclopenta[d]pyridazin-1-yl)amino)piperidine-1-carboxylate

To a solution of tert-butyl (R)-3-((4-chloro-6,7-dihydro-5H-cyclopenta[d]pyridazin-1-yl)amino)piperidine-1-carboxylate (250 mg, 0.7 mmol, 1 eq.) and 4-fluoro-2-hydroxyphenylboronic acid (133 mg, 0.8 mmol, 1.2 eq.) in a mixture of 1,4-dioxane (3 mL) and water (0.8 mL) were added Pd(PPh₃)₄ (41 mg, 0.04 mmol, 0.05 eq.) and solid NaHCO₃ (119 mg, 1.4 mmol, 2.0 eq.). After stirring for 5 h at 100° C. under an atmosphere of N₂ in a sealed tube the resulting mixture was concentrated under reduced pressure using rotary evaporator. The residue was purified by silica gel column chromatography using 0~50% EtOAc/PE gradients to afford tert-butyl (R)-3-((4-(4-fluoro-2-hydroxyphenyl)-6,7-dihydro-5H-cyclopenta[d]pyridazin-1-yl)amino)piperidine-1-carboxylate (250 mg, 82.4% yield) as a light yellow solid after evaporation of the combined product containing fractions under reduced pressure using a rotary evaporator. LC/MS (ESI): mass calcd for C₂₃H₂₉FN₄O₃: 428.22, found: m/z=429.25 [M+H]⁺.

### Step 2: Synthesis of (R)-5-fluoro-2-(4-(piperidin-3-ylamino)-6,7-dihydro-5H-cyclopenta[d]pyridazin-1-yl)phenol

To a light yellow solution of tert-butyl (R)-3-((4-(4-fluoro-2-hydroxyphenyl)-6,7-dihydro-5H-cyclopenta[d]pyridazin-1-yl)amino)piperidine-1-carboxylate (250 mg, 0.6 mmol, 1 eq.) in DCM (5 mL) was added TFA (1 mL). After stirring the reaction mixture for 30 min at rt, the suspension was concentrated under reduced pressure using a rotary evaporator. The residue was purified by reversed flash chromatography using the following conditions: column: C18 silica gel; mobile phase: ACN in water, 10% to 60% gradient in 20 min; detector: UV 254 nm to yield (R)-5-fluoro-2-(4-(piperidin-3-ylamino)-6,7-dihydro-5H-cyclopenta[d]pyridazin-1-yl)phenol (180 mg, 93.9% yield) as light yellow solid after lyophilization of the combined product containing fractions. LC/MS (ESI): mass calcd for C₁₈H₂₁FN₄O: 328.17, found: m/z=329.15 [M+H]⁺.

### Step 3: Synthesis of tert-butyl (R)-3-(3-((4-(4-fluoro-2-hydroxyphenyl)-6,7-dihydro-5H-cyclopenta[d]pyridazin-1-yl)amino)piperidin-1-yl)azetidine-1-carboxylate

To a light yellow solution of 5-fluoro-2-{4-[(3R)-piperidin-3-ylamino]-5H,6H,7H-cyclopenta[d]pyridazin-1-yl}phenol (90 mg, 0.3 mmol, 1.0 eq.) in MeOH (2 mL) were added tert-butyl 3-oxoazetidine-1-carboxylate (56 mg, 0.3 mmol, 1.2 eq.) and NaBH₃CN (52 mg, 0.8 mmol, 3.0 eq.). After stirring the reaction mixture for 2 h at rt, the suspension was concentrated under reduced pressure using a rotary evaporator. The residue was purified by silica gel column chromatography using a 0~60% EtOAc/PE gradient to afford tert-butyl (R)-3-(3-((4-(4-fluoro-2-hydroxyphenyl)-6,7-dihydro-5H-cyclopenta[d]pyridazin-1-yl)amino)piperidin-1-yl)azetidine-1-carboxylate (101 mg, 76.2% yield) as a light yellow solid after evaporation of the combined product containing fractions under reduced pressure using a rotary evaporator. LC/MS (ESI): mass calcd for C₂₆H₃₄FN₅O₃: 483.26, found: m/z=484.80 [M+H]⁺.

### Step 4: Synthesis of (R)-2-(4-((1-(azetidin-3-yl)piperidin-3-yl)amino)-6,7-dihydro-5H-cyclopenta[d]pyridazin-1-yl)-5-fluorophenol (81)

To a light yellow solution of tert-butyl (R)-3-(3-((4-(4-fluoro-2-hydroxyphenyl)-6,7-dihydro-5H-cyclopenta[d]pyridazin-1-yl)amino)piperidin-1-yl)azetidine-1-carboxylate (100 mg, 0.2 mmol, 1.0 eq.) in DCM (5 mL) was added TFA (1 mL). After stirring the reaction mixture for 30 min at rt, the suspension was concentrated under reduced pressure using a rotary evaporator. The residue was purified by reversed flash chromatography using the following conditions: Column: XBridge Prep Phenyl OBD Column, 19*100 mm, 5 μm; Mobile Phase A: Water (10 mmol/L NH₄HCO₃+0.1% NH₃), Mobile Phase B: ACN; Flow rate: 60 mL/min; Gradient: 5% B to 95% B in 7 min, 95% B; Wavelength: 254 nm; RT: 6 min, yielding the target compound (R)-2-(4-((1-(azetidin-3-yl)piperidin-3-yl)amino)-6,7-dihydro-5H-cyclopenta[d]pyridazin-1-yl)-5-fluorophenol (81) (5.4 mg, 6.7% yield) as white solid after lyophilization of the combined product containing fractions. LC/MS (ESI): mass calcd for C₂₁H₂₆FN₅O: 383.21, found: m/z=384.20 [M+H]⁺. ¹H NMR (300 MHz, Methanol-d₄): δ 7.44-7.35 (m, 1H), 6.83-6.74 (m, 2H), 4.89-4.81 (m, 5H), 3.89-3.76 (m, 1H), 3.30-3.19 (m, 1H), 3.08-3.02 (m, 4H), 2.91-2.83 (m, 1H), 2.33-2.23 (m, 4H), 2.19-2.08 (m, 1H), 1.98-1.93 (m, 1H), 1.87-1.62 (m, 2H). ¹⁹F NMR (282 MHz, Methanol-d₄): δ −113.25 ppm.

### Example 82: (R)-1-(2-Amino-7-azaspiro[3.5]nonan-7-yl)-2-(3-((6-(2-hydroxy-4-(trifluoromethyl)phenyl)pyridazin-3-yl)amino)piperidin-1-yl)ethan-1-one (82)

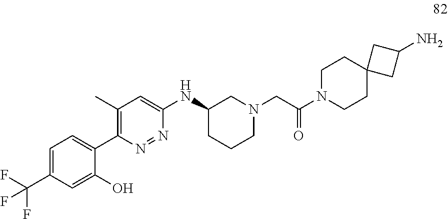 [SMILES: Cc1cc(N[C@@H]2CCCN(CC(=O)N3CCC4(CC3)CC(N)C4)C2)nnc1-c1ccc(C(F)(F)F)cc1O]

### Synthetic Route:

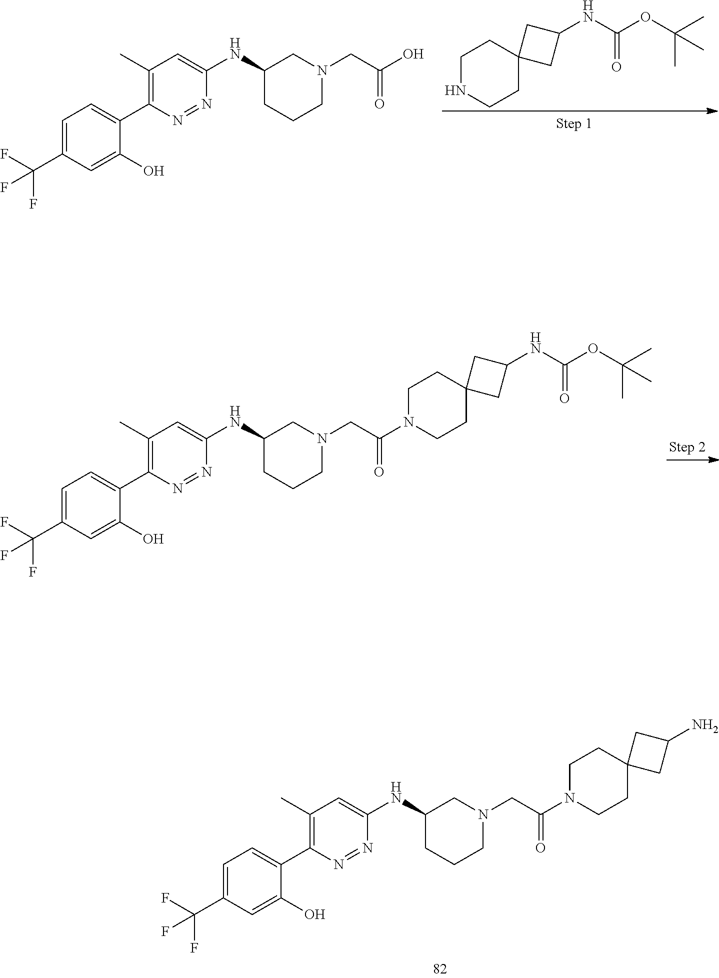 [SMILES: Cc1cc(N[C@@H]2CCCN(CC(=O)O)C2)nnc1-c1ccc(C(F)(F)F)cc1O ; CC(C)(C)OC(=O)NC1CC2(CCNCC2)C1 ; Cc1cc(N[C@@H]2CCCN(CC(=O)N3CCC4(CC3)CC(NC(=O)OC(C)(C)C)C4)C2)nnc1-c1ccc(C(F)(F)F)cc1O ; Cc1cc(N[C@@H]2CCCN(CC(=O)N3CCC4(CC3)CC(N)C4)C2)nnc1-c1ccc(C(F)(F)F)cc1O]

### Step 1: Synthesis of tert-butyl (R)-(7-(2-(3-((6-(2-hydroxy-4-(trifluoromethyl)phenyl)-5-methylpyridazin-3-yl)amino)piperidin-1-yl)acetyl)-7-azaspiro[3.5]nonan-2-yl)carbamate

To a stirred solution of (R)-2-(3-((6-(2-hydroxy-4-(trifluoromethyl)phenyl)-5-methylpyridazin-3-yl)amino)piperidin-1-yl)acetic acid (220 mg, 0.5 mmol, 1.0 eq.) in pyridine (3 mL) was added tert-butyl (7-azaspiro[3.5]nonan-2-yl)carbamate (129 mg, 0.5 mmol, 1.0 eq.) and EDCI (154 mg, 0.8 mmol, 1.5 eq.) in portions at rt. The resulting reaction mixture was stirred for 16 h at 50° C. (oil bath). The reaction mixture was concentrated under reduced pressure using a rotary evaporator, and the residue was purified by silica gel column chromatography eluting with DCM/MeOH gradients (0~10%) to afford tert-butyl (R)-(7-(2-(3-((6-(2-hydroxy-4-(trifluoromethyl)phenyl)-5-methylpyridazin-3-yl)amino)piperidin-1-yl)acetyl)-7-azaspiro[3.5]nonan-2-yl)carbamate (120 mg, 35.3%) as a yellow solid after evaporation of the combined product containing fractions under reduced pressure using a rotary evaporator. LC/MS (ESI): mass calcd, for C₃₂H₄₃F₃N₆O₄: 632.33, Found: m/z=633.35 [M+H]⁺.

### Step 2: Synthesis of (R)-1-(2-amino-7-azaspiro[3.5]nonan-7-yl)-2-(3-((6-(2-hydroxy-4-(trifluoromethyl)phenyl)pyridazin-3-yl)amino)piperidin-1-yl)ethan-1-one (6)

To a stirred solution of tert-butyl (R)-(7-(2-(3-((6-(2-hydroxy-4-(trifluoromethyl)phenyl)-5-methylpyridazin-3-yl)amino)piperidin-1-yl)acetyl)-7-azaspiro[3.5]nonan-2-yl)carbamate (100 mg, 0.2 mmol, 1.0 eq.) in THF (1 mL) was dropwise added HCl (1 mL, 4 M in 1,4-dioxane, 20 eq.) at rt. The resulting reaction mixture was stirred for 3 h at rt followed by concentration under reduced pressure using a rotary evaporator. The crude product (90 mg) was purified by preparative RP-HPLC using the following conditions: Column: XBridge Shield RP 18 OBD Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 26% B to 56% B in 7 min; Wavelength: 254 nm; RT: 6.33 min to afford the target compound (R)-1-(2-amino-7-azaspiro[3.5]nonan-7-yl)-2-(3-((6-(2-hydroxy-4-(trifluoromethyl)phenyl)pyridazin-3-yl)amino)piperidin-1-yl)ethan-1-one (82) (42 mg, 47.4%) as a white solid after lyophilization of the combined product containing fractions. LC/MS (ESI): mass calcd, for C₂₇H₃₅F₃N₆O₂: 532.28. Found: m/z=533.20 [M+H]⁺. ¹H NMR (400 MHz, Methanol-d₄): δ 7.41 (d, J=8.0 Hz, 1H), 7.24 (d, J=8.0 Hz, 1H), 7.17 (s, 1H), 6.79 (s, 1H), 4.21-4.12 (m, 1H), 3.80-3.45 (m, 6H), 3.28-3.20 (m, 1H), 3.95-3.50 (m, 2H), 2.40-2.20 (m, 4H), 2.15 (s, 3H), 2.00-1.80 (m, 2H), 1.75-1.44 (m, 8H). ¹⁹F NMR (376 MHz, Methanol-d₄): δ −64.29 ppm.

### Example 83: (R)-1-(4-Aminopiperidin-1-yl)-2-(3-((6-(2-hydroxy-4-(trifluoromethyl)phenyl)-5-methylpyridazin-3-yl)amino)piperidin-1-yl)ethan-1-one (83)

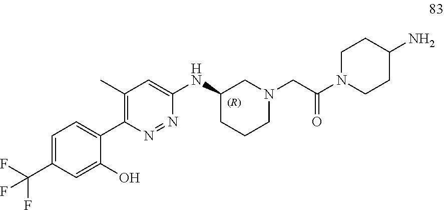

### Synthetic Route:

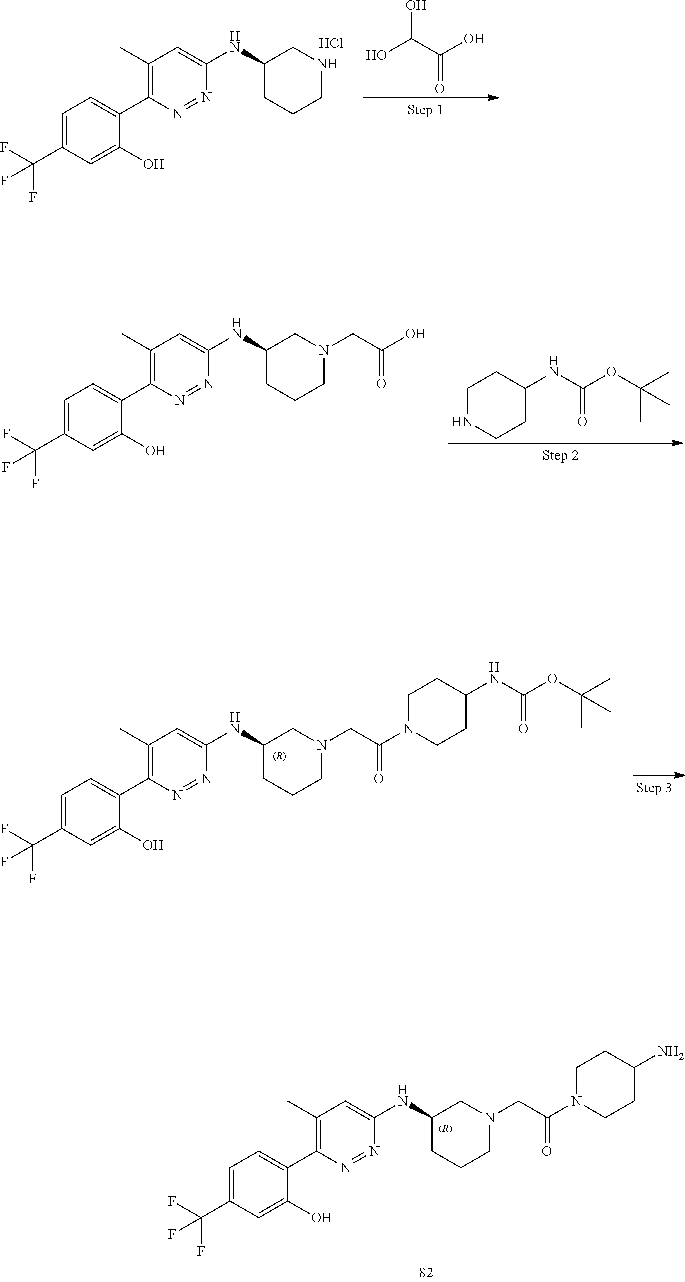

### Step 1: Synthesis of (R)-2-(3-((6-(2-hydroxy-4-(trifluoromethyl)phenyl)-5-methylpyridazin-3-yl)amino)piperidin-1-yl)acetic Acid

To a stirred mixture of (R)-2-(4-methyl-6-(piperidin-3-ylamino)pyridazin-3-yl)-5-(trifluoromethyl)phenol hydrochloride (600 mg, 1.5 mmol, 1.0 eq.) and 2,2-dihydroxyacetic acid (300 mg, 3.3 mmol, 2.1 eq.) in MeOH (7 mL) were added NaBH₃CN (250.0 mg, 3.9 mmol, 2.6 eq.) at 0° C. The resulting mixture was stirred at rt for 16 h. The resulting mixture was quenched with water (2 mL) and concentrated under reduced pressure using a rotary evaporator. The residue was dissolved in DMF (3 mL) and purified by reversed-phase flash chromatography using the following conditions: column, C18 silica gel; mobile phase, ACN in Water (10 mmol/L NH₄HCO₃ aq.), 10% to 50% gradient in 20 min; UV detector: UV 254 nm, to afford (R)-2-(3-((6-(2-hydroxy-4-(trifluoromethyl)phenyl)-5-methylpyridazin-3-yl)amino)piperidin-1-yl)acetic acid (290 mg, 45.8% yield) as a white solid after lyophilization of the combined product containing fractions. LC/MS (ESI): mass calcd. for C₁₉H₂₁F₃N₄O₃: 410.16. Found: m/z=411.15 [M+H]⁺.

### Step 2: Synthesis of tert-butyl (R)-(1-(2-(3-((6-(2-hydroxy-4-(trifluoromethyl)phenyl)-5-methylpyridazin-3-yl)amino)piperidin-1-yl)acetyl)piperidin-4-yl)carbamate

To a stirred mixture of (R)-2-(3-((6-(2-hydroxy-4-(trifluoromethyl)phenyl)-5-methylpyridazin-3-yl)amino)piperidin-1-yl)acetic acid (140 mg, 0.3 mmol, 1.0 eq.) and tert-butyl piperidin-4-ylcarbamate (81 mg, 0.4 mmol, 1.2 eq.) in pyridine (3 mL) was added EDCI (98 mg, 0.5 mmol, 1.5 eq.) at rt. The resulting reaction mixture was stirred for an additional 16 h at 50° C. (oil bath). Upon cooling to rt, the reaction mixture was concentrated under reduced pressure using a rotary evaporator. The residue was purified by silica gel column chromatography eluting using PE/EtOAc gradients (0~50%) to afford tert-butyl (R)-(1-(2-(3-((6-(2-hydroxy-4-(trifluoromethyl)phenyl)-5-methylpyridazin-3-yl)amino)piperidin-1-yl)acetyl)piperidin-4-yl)carbamate (110 mg, 54.4%) as a yellow solid after evaporation of the combined product containing fractions under reduced pressure using a rotary evaporator. LC/MS (ESI): mass calcd for C₂₉H₃₉F₃N₆O₄: 592.30. Found: m/z=593.40 [M+H]⁺.

### Step 3: Synthesis of (R)-1-(4-Aminopiperidin-1-yl)-2-(3-((6-(2-hydroxy-4-(trifluoromethyl)phenyl)-5-methylpyridazin-3-yl)amino)piperidin-1-yl)ethan-1-one (83)

To a stirred mixture of tert-butyl (R)-(1-(2-(3-((6-(2-hydroxy-4-(trifluoromethyl)phenyl)-5-methylpyridazin-3-yl)amino)piperidin-1-yl)acetyl)piperidin-4-yl)carbamate (110 mg, 0.2 mmol, 1.0 eq.) in EA (3 mL) was added HCl (2 mL, 4 M in dioxane, 8 mmol, 40 eq.) at rt. The resulting reaction mixture was stirred overnight at rt and was concentrated under reduced pressure using a rotary evaporator. The crude product was purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 18% B to 48% B in 7 min; Wavelengths: 254 nm/220 nm; RT: 5.17 min. The product containing fractions were combined and lyophilized to afford the target compound (R)-1-(4-Aminopiperidin-1-yl)-2-(3-((6-(2-hydroxy-4-(trifluoromethyl)phenyl)-5-methylpyridazin-3-yl)amino)piperidin-1-yl)ethan-1-one (83) (26 mg, 28.1% yield) as a light yellow solid. LC/MS (ESI): mass calcd for C₂₄H₃₁F₃N₆O₂: 492.25. Found: m/z=493.25 [M+H]⁺. ¹H NMR (400 MHz, Methanol-d₄): δ 7.40-7.10 (m, 3H), 6.82-6.75 (m, 1H), 4.60-4.40 (m, 2H), 4.22-4.10 (m, 1H), 3.70-3.60 (m, 1H), 3.30-3.05 (m, 4H), 2.95-2.60 (m, 2H), 2.50-2.10 (m, 5H), 2.02-1.65 (m, 6H), 1.60-1.35 (m, 2H). ¹⁹F NMR (376 MHz, Methanol-d₄): δ −64.23 ppm.

### Example 84: (R)-2-(6-((1-(Azetidin-3-yl)piperidin-3-yl)amino)-4-methylpyridazin-3-yl)-5-(trifluoromethyl)phenol (84)

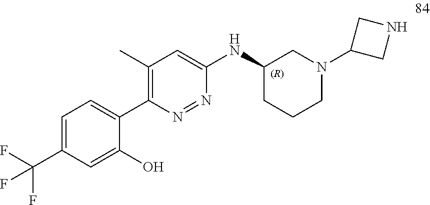

### Synthetic Route:

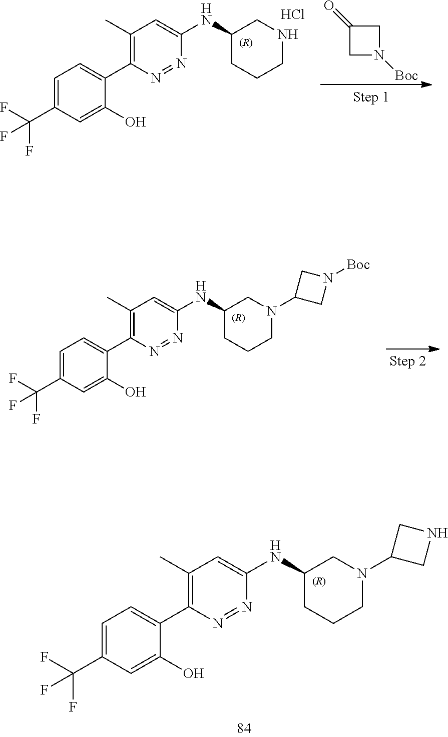

### Step 1: Synthesis of tert-butyl (R)-3-(3-((6-(2-hydroxy-4-(trifluoromethyl)phenyl)-5-methylpyridazin-3-yl)amino)piperidin-1-yl)azetidine-1-carboxylate

To a solution of (R)-2-(4-methyl-6-(piperidin-3-ylamino)pyridazin-3-yl)-5-(trifluoromethyl)phenol hydrochloride (150 mg, 0.39 mmol, 1.0 eq.) and tert-butyl 3-oxoazetidine-1-carboxylate (133 mg, 0.77 mmol, 2.0 eq.) in MeOH (3 mL) was added solid NaBH₃CN (73 mg, 1.16 mmol, 3.0 eq.). The resulting solution was stirred for 2 h at rt. The reaction mixture was concentrated under reduced pressure using a rotary evaporator. The residue purified by silica gel column chromatography eluting with DCM/MeOH (0~20%) to afford tert-butyl (R)-3-(3-((6-(2-hydroxy-4-(trifluoromethyl)phenyl)-5-methylpyridazin-3-yl)amino)piperidin-1-yl)azetidine-1-carboxylate as a yellow oil (160 mg, 74.4% yield) after evaporation of the combined product containing fractions under reduced pressure using a rotary evaporator. LC/MS: mass calcd: C₂₅H₃₂F₃N₅O₃: 507.25, found: m/z=508.20 [M+H]⁺.

### Step 2: Synthesis of (R)-2-(6-((1-(azetidin-3-yl)piperidin-3-yl)amino)-4-methylpyridazin-3-yl)-5-(trifluoromethyl)phenol (84)

To a solution of tert-butyl (R)-3-(3-((6-(2-hydroxy-4-(trifluoromethyl)phenyl)-5-methylpyridazin-3-yl)amino)piperidin-1-yl)azetidine-1-carboxylate (150 mg, 0.30 mmol, 1.0 eq.) in DCM (1.5 mL) was added TFA (1.5 mL) at 0° C. (ice bath). The resulting reaction mixture was stirred for 2 h with warming to rt followed by concentration under reduced pressure using a rotary evaporator. The residue was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min; Wavelength: 254 nm; RT1: 4.57 min. After lyophilization of the combined product containing fractions, the target compound (R)-2-(6-((1-(azetidin-3-yl)piperidin-3-yl)amino)-4-methylpyridazin-3-yl)-5-(trifluoromethyl)phenol (84) was obtained as a white solid (21.7 mg, 17.7% yield). LC/MS: mass calcd for C₂₀H₂₄F₃N₅O: 407.19, found: m/z=408.20 [M+H]⁺. ¹H NMR (400 MHz, DMSO-d₆) δ ppm: 7.40-7.37 (m, 1H), 7.24-7.20 (m, 2H), 6.69 (s, 1H), 4.01-3.97 (m, 1H), 3.49-3.43 (m, 3H), 3.14-3.10 (m, 1H), 2.85-2.82 (m, 1H), 2.49-2.37 (m, 2H), 2.01 (s, 3H), 1.89-1.81 (m, 2H), 1.78-1.69 (m, 2H), 1.55-1.51 (m, 1H), 1.49-1.28 (m, 1H) ppm. ¹⁹F NMR (376 MHz, DMSO-d₆): δ −61.15 ppm.

### Example 85: (R)-2-(6-([1,4′-Bipiperidin]-3-ylamino)-4-methylpyridazin-3-yl)-5-(trifluoromethyl)phenol (85)

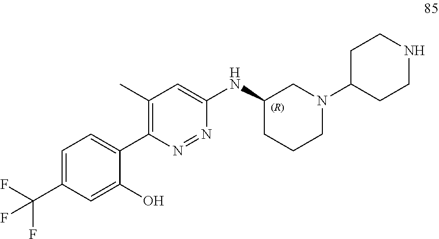

### Synthetic Route:

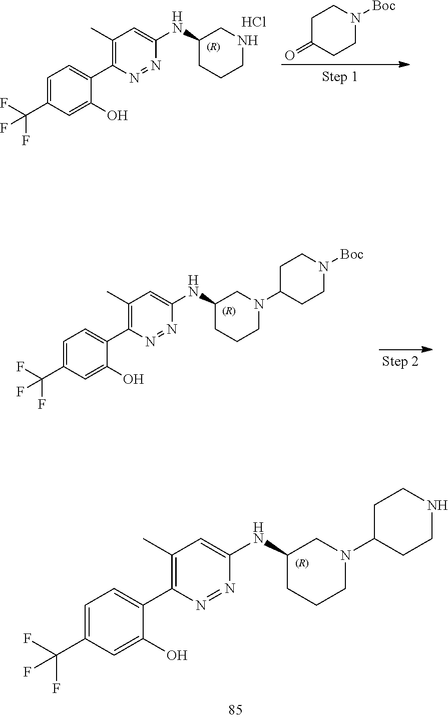

### Step 1: Synthesis of tert-butyl (R)-3-((6-(2-hydroxy-4-(trifluoromethyl)phenyl)-5-methylpyridazin-3-yl)amino)-[1,4′-bipiperidine]-1′-carboxylate

A colorless suspension of (R)-2-(4-methyl-6-(piperidin-3-ylamino)pyridazin-3-yl)-5-(trifluoromethyl)phenol hydrochloride (300 mg, 0.772 mmol, 1.0 eq.) and tert-butyl 4-oxopiperidine-1-carboxylate (230 mg, 1.16 mmol, 1.5 eq.) in MeOH (4 mL) was stirred for 0.5 h at rt. To this suspension solid NaBH₃CN (97 mg, 1.54 mmol, 2.0 eq.) was added leading to evolution of gas and a colorless solution which was stirred overnight at rt. The reaction was quenched by addition of water (5 mL). The mixture obtained was extracted with EtOAc (2×20 mL). The combined organic phases were washed with brine (2×10 mL), dried with anhydrous Na₂SO₄, filtered, and the filtrate concentrated under reduced pressure using a rotary evaporator. The residue was purified by silica gel column chromatography to afford tert-butyl (R)-3-((6-(2-hydroxy-4-(trifluoromethyl)phenyl)-5-methylpyridazin-3-yl)amino)-[1,4′-bipiperidine]-1′-carboxylate as yellow oil (340 mg, 73.0% yield) after evaporation of the combined product containing fractions under reduced pressure using a rotary evaporator. LC/MS: mass calcd for C₂₇H₃₆F₃N₅O₃: 535.28, found: m/z=536.25 [M+H]⁺.

### Step 2: Synthesis of (R)-2-(6-([1,4′-bipiperidin]-3-ylamino)-4-methylpyridazin-3-yl)-5-(trifluoromethyl)phenol (85)

To a solution of tert-butyl (R)-3-((6-(2-hydroxy-4-(trifluoromethyl)phenyl)-5-methylpyridazin-3-yl)amino)-[1,4′-bipiperidine]-1′-carboxylate (200 mg, 0.37 mmol, 1.0 eq.) in DCM (2 ml) was added HCl (1 mL, 4 M in 1,4-dioxane, 4 mmol, ~11 eq.). The solution was stirred for 3 h at rt. After concentration under reduced pressure using a rotary evaporator, the residue was purified by to preparative RP-HPLC using the following conditions: Column: XSelect CSH Prep C18 OBD Column, 19*250 mm, 5 μm; Mobile Phase A: Water (0.1% FA), Mobile Phase B: ACN; Flow rate: 25 mL/min; Gradient: 6% B to 36% B in 7 min, 36% B; Wavelength: 254 nm; RT1: 5 min. After lyophilization of the combined product containing fractions, the target compound (R)-2-(6-([1,4′-bipiperidin]-3-ylamino)-4-methylpyridazin-3-yl)-5-(trifluoromethyl)phenol (85) format salt was obtained as a white solid (15.0 mg, 8.2% yield). LC/MS: mass calcd for C₂₃H₃₀F₃N₅O₃: 435.22, found: m/z=436.2 [M+H]⁺. ¹H NMR (300 MHz, Methanol-d₄): δ 8.32 (s, 1H), 7.41 (d, J=7.9 Hz, 1H), 7.26 (d, J=8.0 Hz, 1H), 7.20 (s, 1H), 6.85 (s, 1H), 4.23-4.17 (m, 1H), 3.59-3.39 (m, 3H), 3.13-2.93 (m, 4H), 2.73-2.60 (m, 1H), 2.57-2.45 (m, 1H), 2.28-2.13 (m, 5H), 2.11-1.75 (m, 5H), 1.63-1.54 (m, 1H) ppm. ¹⁹F NMR (282 MHz, Methanol-d₄): δ −64.32 ppm.

### Example 86: (R)-(3-((6-(2-hydroxy-4-(trifluoromethyl)phenyl)-5-methylpyridazin-3-yl)amino)-[1,4′-bipiperidin]-1′-yl) (piperazin-1-yl)methanone (86)

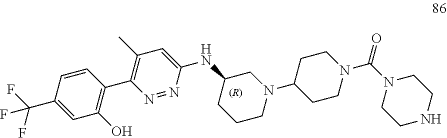

### Synthetic Route:

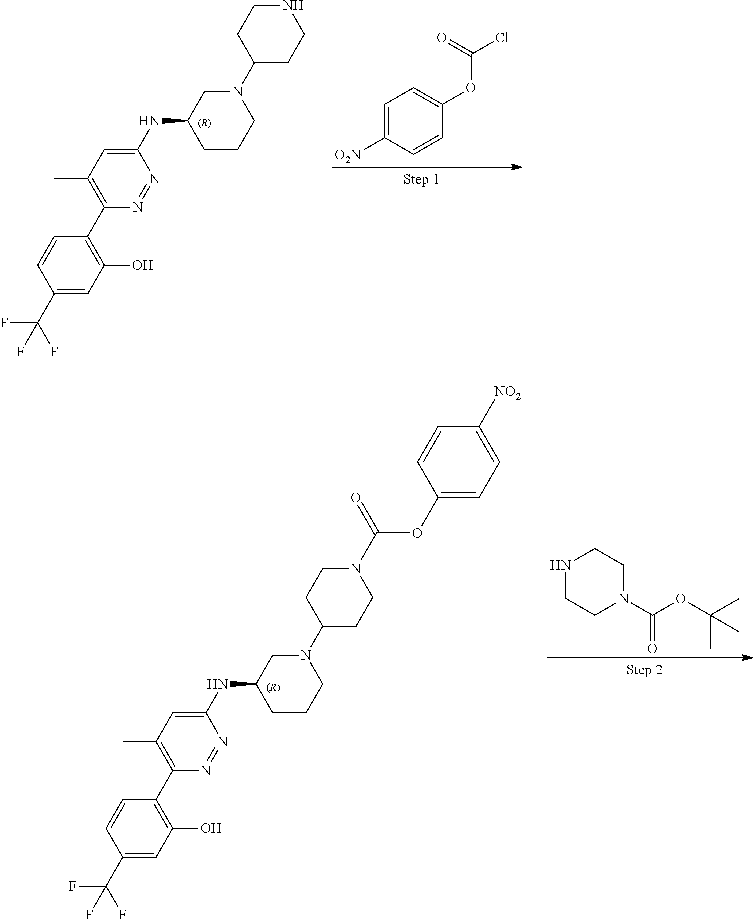

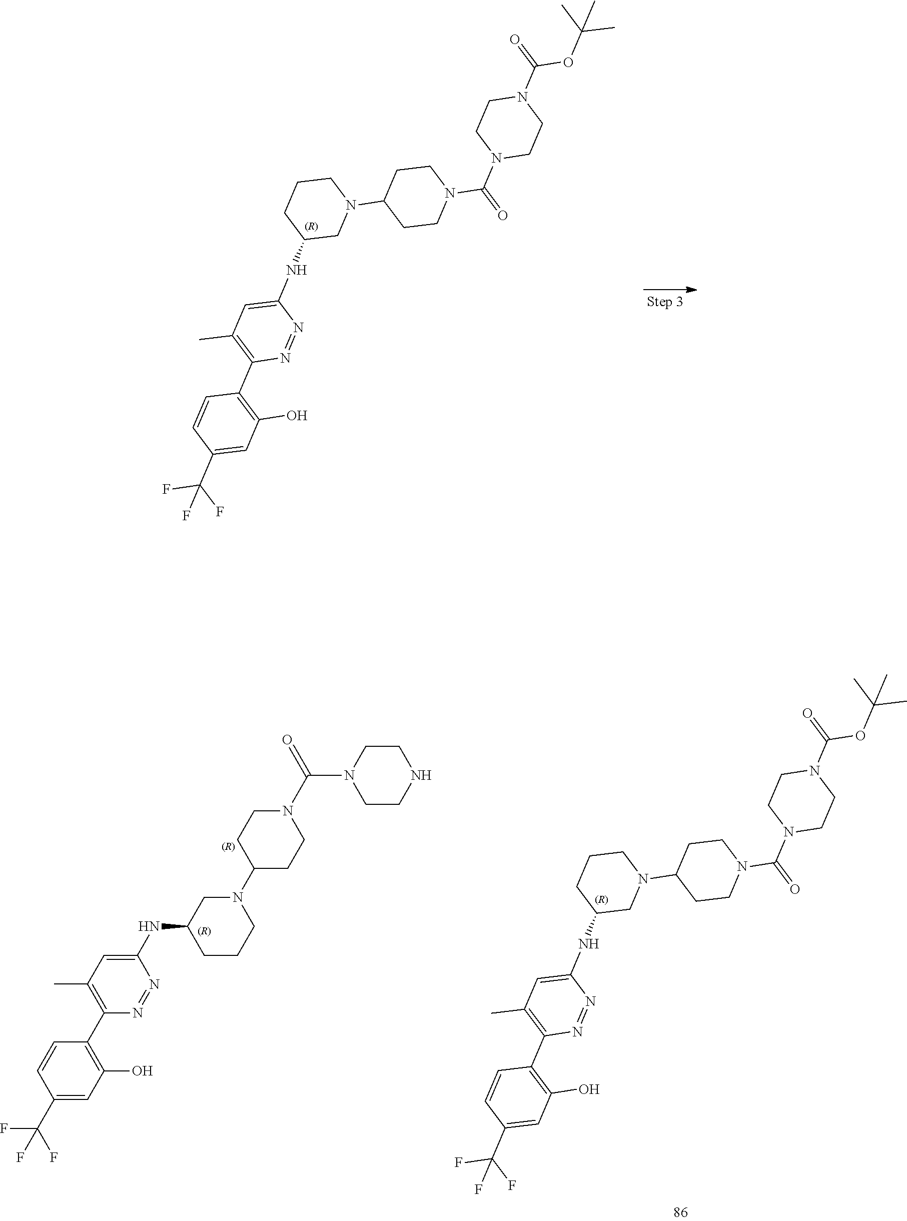

### Step 1: Synthesis of 4-nitrophenyl (R)-3-((6-(2-hydroxy-4-(trifluoromethyl)phenyl)-5-methylpyridazin-3-yl)amino)-[1,4′-bipiperidine]-1′-carboxylate

A solution of (R)-2-(6-([1,4′-bipiperidin]-3-ylamino)-4-methylpyridazin-3-yl)-5-(trifluoromethyl)phenol (1.8 g, 4.1 mmol, 1.0 eq.) in DCM (20 mL) was treated with 4-nitrophenyl carbonochloridate (4-nitrophenyl chloroformate) (0.9 g, 4.5 mmol, 1.1 eq.) followed by dropwise addition of TEA (2.34 mL, 1.7 g, 16.5 mmol, 4.0 eq.) at 0° C. (ice bath). The reaction mixture was stirred at rt for overnight. The reaction was quenched by the addition of water (5 mL) at 0° C. (ice bath). DCM (20 mL) and water (15 mL) were added, and the resulting mixture was extracted with DCM (3×20 mL). The combined organic extracts were dried with anhydrous Na₂SO₄, filtered, and the filtrate was concentrated under reduced pressure using a rotary evaporator. The residue was purified by silica gel column chromatography eluting with MeOH/DCM gradients (0~5%) to afford 4-nitrophenyl (R)-3-((6-(2-hydroxy-4-(trifluoromethyl)phenyl)-5-methylpyridazin-3-yl)amino)-[1,4′-bipiperidine]-1′-carboxylate (800 mg, 32.2% yield) as a yellow solid after evaporation of the combined product containing fractions under reduced pressure using a rotary evaporator. LC/MS (ESI): mass calcd for C₃₂H₄₄F₃N₇O₄: 600.23, found: m/z=601.25 [M+H]⁺.

### Step 2: Synthesis of tert-butyl (R)-4-(3-((6-(2-hydroxy-4-(trifluoromethyl)phenyl)-5-methylpyridazin-3-yl)amino)-[1,4′-bipiperidine]-1′-carbonyl) piperazine-1-carboxylate

To a stirred solution of 4-nitrophenyl (R)-3-((6-(2-hydroxy-4-(trifluoromethyl)phenyl)-5-methylpyridazin-3-yl)amino)-[1,4′-bipiperidine]-1′-carboxylate (250 mg, 0.4 mmol, 1.0 eq.) in ACN (3 mL) were added tert-butyl piperazine-1-carboxylate (78 mg, 0.4 mmol, 1.0 eq.) and DIEA (108 mg, 145 μL, 0.8 mmol, 2.0 eq.) in portions at 0° C. (ice bath). The resulting reaction mixture was stirred for 16 h at 80° C. (oil bath). After filtration, the filtrate was concentrated under reduced pressure using a rotary evaporator. The residue was purified by silica gel column chromatography eluting with DCM/MeOH gradients (0~5%) to afford tert-butyl (R)-4-(3-((6-(2-hydroxy-4-(trifluoromethyl)phenyl)-5-methylpyridazin-3-yl)amino)-[1,4′-bipiperidine]-1′-carbonyl) piperazine-1-carboxylate (80 mg, 29.7% yield) as a white solid after evaporation of the combined product containing fractions under reduced pressure using a rotary evaporator. LC/MS (ESI): mass calcd for C₃₂H₄₄F₃N₇O₄: 647.34, found: m/z=648.30 [M+H]⁺.

### Step 3: Synthesis of (R)-(3-((6-(2-hydroxy-4-(trifluoromethyl)phenyl)-5-methylpyridazin-3-yl)amino)-[1,4′-bipiperidin]-1′-yl) (piperazin-1-yl)methanone (86)

To a stirred solution of tert-butyl (R)-4-(3-((6-(2-hydroxy-4-(trifluoromethyl)phenyl)-5-methylpyridazin-3-yl)amino)-[1,4′-bipiperidine]-1′-carbonyl) piperazine-1-carboxylate (80 mg, 0.1 mmol, 1.0 eq.) in THF (2.0 mL) was dropwise added a solution of HCl (1 mL, 4 M in 1,4-dioxane, 4 mmol, 40 eq.) at rt, and the resulting reaction mixture was stirred for 3 h at rt. The reaction mixture was concentrated under reduced pressure using a rotary evaporator. The residue was purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 20% B to 34% B in 10 min; Wavelengths: 254 nm/220 nm; RT: 10 min to afford the target compound (R)-(3-((6-(2-hydroxy-4-(trifluoromethyl)phenyl)-5-methylpyridazin-3-yl)amino)-[1,4′-bipiperidin]-1′-yl) (piperazin-1-yl)methanone (86) (23.8 mg, 33.5% yield) as a white solid after lyophilization of the combined product containing fractions. LC/MS (ESI): mass calcd for C₂₇H₃₆F₃N₇O₂: 547.29, found: m/z=548.30 [M+H]⁺. ¹H NMR (400 MHz, Methanol-d₄): δ 7.41 (d, J=8.0 Hz, 1H), 7.24-7.22 (m, 1H), 7.18 (s, 1H), 6.78 (s, 1H), 4.18-4.08 (m, 1H), 3.88-3.78 (m, 2H), 3.30-3.20 (m, 5H), 2.90-2.790 (m, 6H), 2.78-2.60 (m, 1H), 2.65-2.55 (m, 1H), 2.50-2.40 (m, 1H), 2.34-2.24 (m, 1H), 2.15 (s, 3H), 2.10-1.98 (m, 1H), 1.92-1.79 (m, 3H), 1.70-1.60 (m, 1H), 1.58-1.45 (m, 3H) ppm. ¹⁹F NMR (376 MHz, Methanol-d₄): δ −64.29 ppm.

### Example 87: (R)-(4-Aminopiperidin-1-yl) (3-((6-(2-hydroxy-4-(trifluoromethyl)phenyl)-5-methylpyridazin-3-yl)amino)-[1,4′-bipiperidin]-1′-yl)methanone (87)

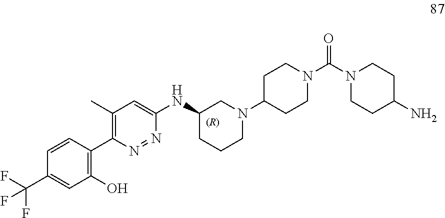

### Synthetic Route:

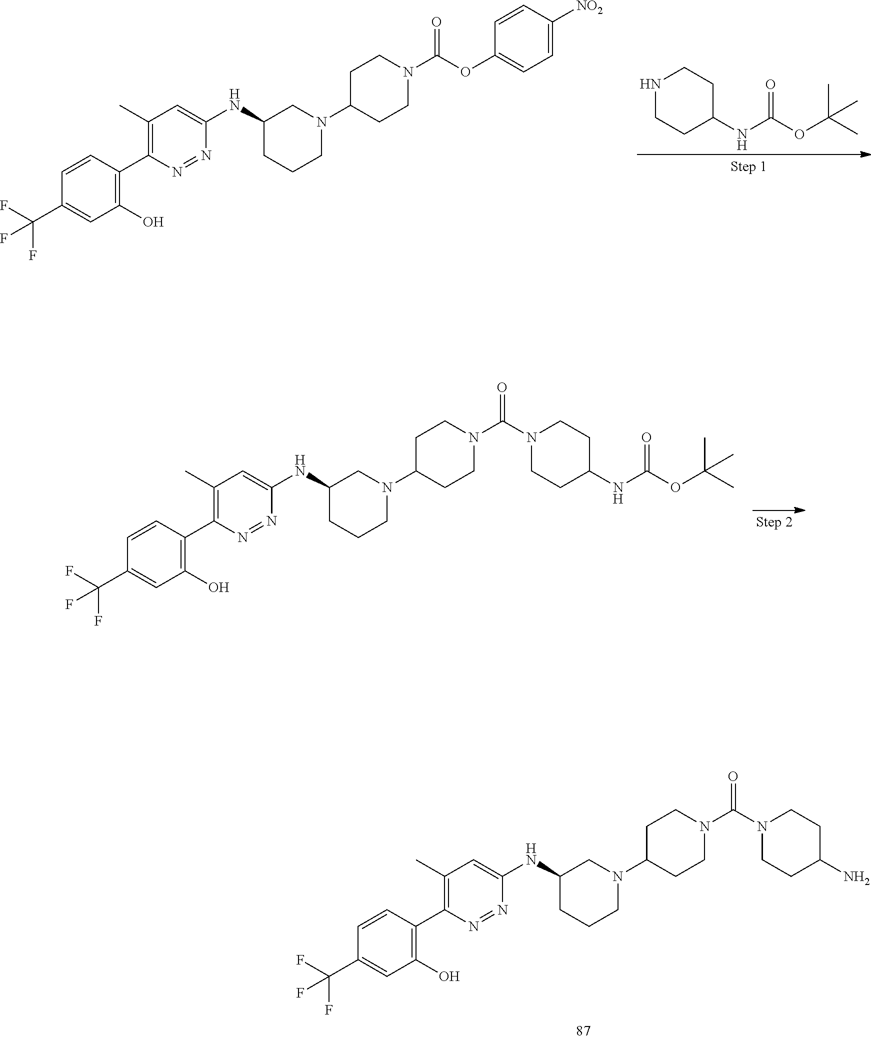 [SMILES: Cc1cc(N[C@@H]2CCCN(C3CCN(C(=O)Oc4ccc([N+](=O)[O-])cc4)CC3)C2)nnc1-c1ccc(C(F)(F)F)cc1O ; CC(C)(C)OC(=O)NC1CCNCC1 ; Cc1cc(N[C@@H]2CCCN(C3CCN(C(=O)N4CCC(NC(=O)OC(C)(C)C)CC4)CC3)C2)nnc1-c1ccc(C(F)(F)F)cc1O ; Cc1cc(N[C@@H]2CCCN(C3CCN(C(=O)N4CCC(N)CC4)CC3)C2)nnc1-c1ccc(C(F)(F)F)cc1O]

### Step 1: Synthesis of tert-butyl (R)-(1-(3-((6-(2-hydroxy-4-(trifluoromethyl)phenyl)-5-methylpyridazin-3-yl)amino)-[1,4′-bipiperidine]-1′-carbonyl)piperidin-4-yl)carbamate

To a stirred mixture of 4-nitrophenyl (R)-3-((6-(2-hydroxy-4-(trifluoromethyl)phenyl)-5-methylpyridazin-3-yl)amino)-[1,4′-bipiperidine]-1′-carboxylate (200 mg, 0.3 mmol, 1.0 eq.) and tert-butyl piperidin-4-ylcarbamate (100 mg, 0.5 mmol, 1.5 eq.) in DMSO (4 mL) was added DIEA (78 mg, 105 μL, 0.6 mmol, 2.0 eq.) at 0° C. (ice batch). The resulting reaction mixture was stirred for 4 h at 100° C. (oil bath). After cooling to rt, the resulting reaction mixture was quenched with water/ice slurry (10 mL) and extracted with EA (3×30 mL). The combined organic layers were washed with H₂O (3×50 mL) and dried over anhydrous Na₂SO₄. After filtration, the filtrate was concentrated under reduced pressure using a rotary evaporator. The residue was purified by silica gel column chromatography eluting with DCM/MeOH gradients (0~10%) to afford (R)-(1-(3-((6-(2-hydroxy-4-(trifluoromethyl)phenyl)-5-methylpyridazin-3-yl)amino)-[1,4′-bipiperidine]-1′-carbonyl)piperidin-4-yl)carbamate (150 mg, 68.0% yield) as a yellow solid after evaporation of the combined product containing fractions under reduced pressure using a rotary evaporator. LC/MS (ESI): mass calcd for C₃₃H₄₆F₃N₇O₄: 661.36, found: m/z=662.50 [M+H]⁺.

### Step 2: Synthesis of (R)-(4-aminopiperidin-1-yl) (3-((6-(2-hydroxy-4-(trifluoromethyl)phenyl)-5-methylpyridazin-3-yl)amino)-[1,4′-bipiperidin]-1′-yl)methanone (87)

To a stirred mixture of tert-butyl (R)-(1-(3-((6-(2-hydroxy-4-(trifluoromethyl)phenyl)-5-methylpyridazin-3-yl)amino)-[1,4′-bipiperidine]-1′-carbonyl)piperidin-4-yl)carbamate (150 mg, 0.2 mmol, 1.0 eq.) in THF (3 mL) was added a solution of HCl (1.5 mL, 4 M in 1,4-dioxane, 6 mmol, ~30 eq.) at 0° C. (ice bath). The resulting reaction mixture was stirred overnight with warming to rt. The resulting reaction mixture was concentrated under reduced pressure using a rotary evaporator. The crude product was purified by preparative HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min; Gradient: 19% B to 33% B in 11 min; Wavelengths: 254 nm/220 nm; RT: 10.67 min to afford the target compound (R)-(4-aminopiperidin-1-yl) (3-((6-(2-hydroxy-4-(trifluoromethyl)phenyl)-5-methylpyridazin-3-yl)amino)-[1,4′-bipiperidin]-1′-yl)methanone (87) (27.0 mg, 20.8%) as an off-white solid after lyophilization of the combined product containing fractions. LC/MS (ESI): mass calcd for C₂₈H₃₈F₃N₇O₂: 561.30, found: m/z=562.35 [M+H]⁺. ¹H NMR (300 MHz, Methanol-d₄): δ 7.37 (d, J=7.6 Hz, 1H), 7.22-7.12 (m, 2H), 6.76 (s, 1H), 4.25-4.15 (m, 1H), 3.80-3.55 (m, 4H), 3.20-3.10 (m, 1H), 2.90-2.70 (m, 6H), 2.60-2.52 (m, 1H), 2.45-2.35 (m, 1H), 2.30-2.20 (m, 1H), 2.12 (s, 3H), 2.05-1.95 (m, 1H), 1.90-1.60 (m, 6H), 1.55-1.35 (m, 5H) ppm. ¹⁹F NMR (282 MHz, Methanol-d₄): δ −63.92 ppm.

### Example 88: (R)-2-(6-((1-(Azetidin-3-yl)piperidin-3-yl)amino)pyridazin-3-yl)-3-methyl-5-(trifluoromethyl)phenol (88)

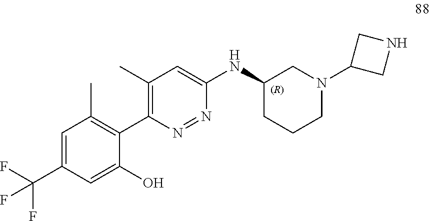

### Synthetic Route:

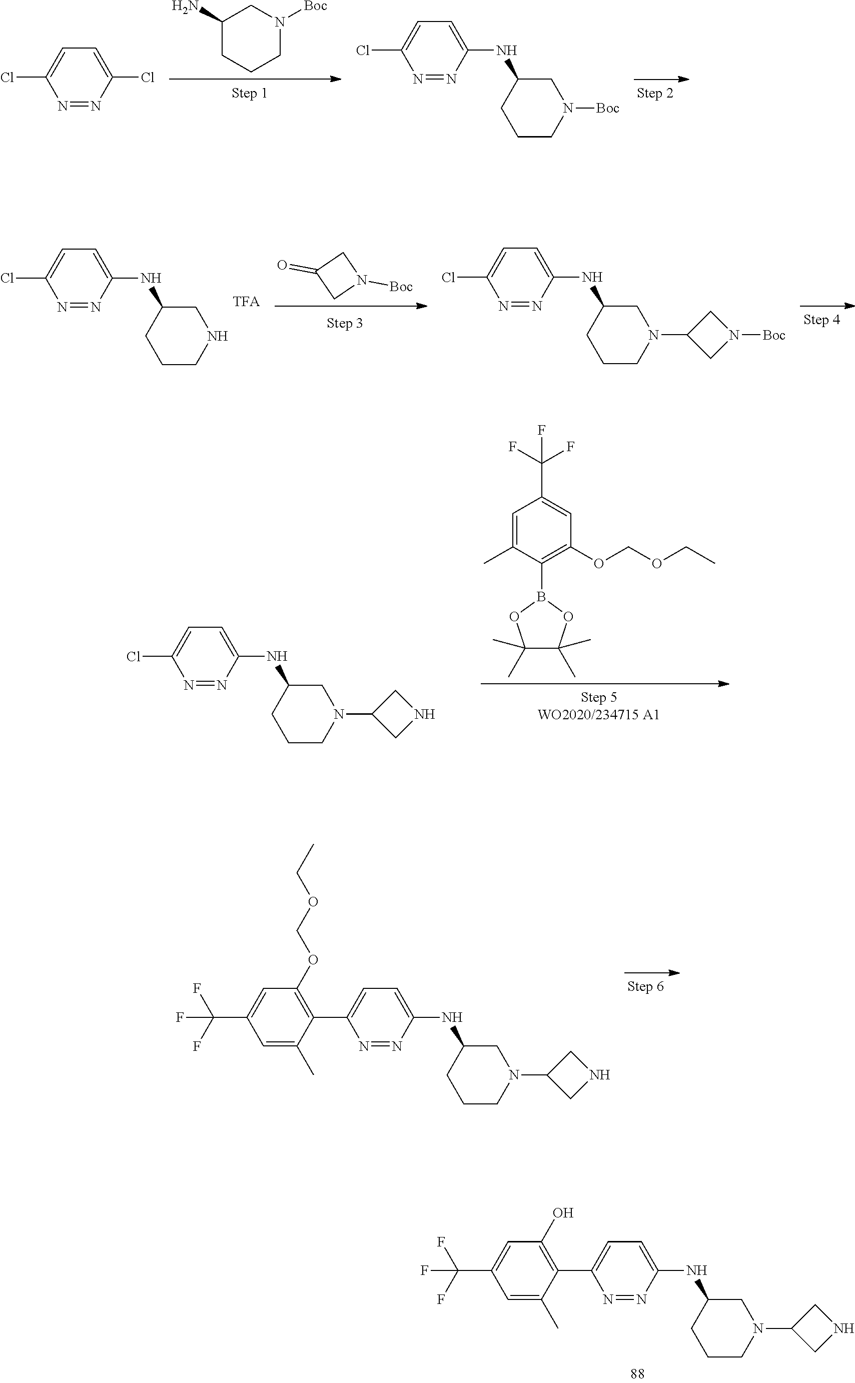

### Step 1: Synthesis of tert-butyl (R)-3-((6-chloropyridazin-3-yl)amino)piperidine-1-carboxylate

To a light yellow solution of 3,6-dichloro-pyridazine (900 mg, 6.0 mmol, 1.0 eq.) in NMP (10 mL) were added tert-butyl (R)-3-aminopiperidine-1-carboxylate (1.45 g, 7.2 mmol, 1.2 eq.) and DIEA (2.11 mL, 1.56 g, 12.1 mmol, 2.0 eq.). After stirring for 12 h at 130° C. in a sealed tube, the reaction mixture was cooled to rt, and the residue was extracted with EA (3×50 mL). The combined organic phases were washed with water (3×50 mL), dried with anhydrous Na₂SO₄, filtered, and the filtrate was concentrated under reduced pressure using a rotary evaporator. The residue was purified by silica gel column chromatography using (0~45% EA/PE) to afford tert-butyl (R)-3-((6-chloropyridazin-3-yl)amino)piperidine-1-carboxylate (600 mg, 31.8% yield) as light yellow solid after evaporation of the product containing fractions under reduced pressure using a rotary evaporator. LC/MS (ESI): mass calcd for C₁₄H₂₁ClN₄O₂: 312.14, found: m/z=313.20 [M+H]⁺.

### Step 2: Synthesis of (R)-6-chloro-N-(piperidin-3-yl)pyridazin-3-amine trifluoro acetate

To a light yellow solution of tert-butyl (3R)-3-[(6-chloropyridazin-3-yl)amino]piperidine-1-carboxylate (500 mg, 1.60 mmol, 1.0 eq.) in DCM (10 mL) was added TFA (2 mL). After stirring for 30 min at rt, the suspension was concentrated under reduced pressure using a rotary evaporator. The residue was purified by reversed phase flash chromatography using the following conditions: column, C18 silica gel; mobile phase, ACN in water, 10% to 60% gradient in 20 min; detector, UV 254 nm to yield (R)-6-chloro-N-(piperidin-3-yl)pyridazin-3-amine trifluoroacetate (200 mg, 58.8% yield) as light yellow solid. LC/MS (ESI): mass calcd for C₉H₁₃ClN₄: 212.08, found: m/z=213.15 [M+H]⁺.

### Step 3: Synthesis of tert-butyl (R)-3-(3-((6-chloropyridazin-3-yl)amino)piperidin-1-yl)azetidine-1-carboxylate

To a solution of (R)-6-chloro-N-(piperidin-3-yl)pyridazin-3-amine trifluoroacetate (2.00 g, 8.0 mmol) and tert-butyl 3-oxoazetidine-1-carboxylate (3.4 g, 20.1 mmol, 2.0 eq.) in MeOH (30 mL) was added solid NaBH₃CN (1.0 g, 16.1 mmol, 2.0 eq.) in small portions. The resulting solution was stirred at rt for overnight, and the reaction mixture was concentrated under reduced pressure using a rotary evaporator. The residue was purified by silica gel column chromatography eluting with EA/PE (0~100%) to afford tert-butyl (R)-3-(3-((6-chloropyridazin-3-yl)amino)piperidin-1-yl)azetidine-1-carboxylate as a yellow solid (1.3 g, 41.8% yield) after evaporation of the combined product containing fractions under reduced pressure using a rotary evaporator. LC/MS (ESI): mass calcd for C₁₇H₂₆ClN₅O₂: 367.18, found: m/z=368.15 [M+H]⁺.

### Step 4: Synthesis of (R)—N-(1-(azetidin-3-yl)piperidin-3-yl)-6-chloropyridazin-3-amine

To a light yellow solution of tert-butyl (R)-3-(3-((6-chloropyridazin-3-yl)amino)piperidin-1-yl)azetidine-1-carboxylate (200 mg, 0.5 mmol, 1 eq.) in DCM (5 mL) were added TFA (1 mL). After stirring for 30 min at rt, the suspension was concentrated under reduced pressure using a rotary evaporator. The residue was purified by reversed flash chromatography using the following conditions: column, C18 silica gel; mobile phase, ACN in water, 10% to 70% gradient in 20 min; detector, UV 254 nm to afford (R)—N-(1-(azetidin-3-yl)piperidin-3-yl)-6-chloropyridazin-3-amine (110 mg, 75.6% yield) as light yellow solid after lyophilization of the combined product containing fractions. LC/MS (ESI): mass calcd for C₁₂H₁₈ClN₅: 267.12, found: m/z=268.15 [M+H]⁺.

### Step 5: Synthesis of (R)—N-(1-(azetidin-3-yl)piperidin-3-yl)-6-(2-(ethoxymethoxy)-6-methyl-4-(trifluoromethyl)phenyl)pyridazin-3-amine

To a solution of (R)—N-(1-(azetidin-3-yl)piperidin-3-yl)-6-chloropyridazin-3-amine (200 mg, 0.7 mmol) in dioxane/H₂O (v/v, 2/1, 3.6 mL/1.8 mL) in a sealed tube were added 2-(2-(ethoxymethoxy)-6-methyl-4-(trifluoromethyl)phenyl)-4,4,5,5-tetramethyl-1,3,2-dioxaborolane (prepared in 4 steps according to PCT patent application publication WO2020/234715 A1) (538 mg, 1.5 mmol, ~2 eq.), Pd(dppf)Cl₂ (54.7 mg, 0.075 mmol, ~0.1 eq.) and solid NaHCO₃ (188 mg, 2.2 mmol, ~3.0 eq.). The resulting reaction mixture was stirred at 150° C. for 1 h under an atmosphere of N₂. After cooling down to rt, the reaction mixture was concentrated under reduced pressure using a rotary evaporator. The residue was purified by silica gel column chromatography eluting with MeOH/DCM (0~20%) to afford (R)—N-(1-(azetidin-3-yl)piperidin-3-yl)-6-(2-(ethoxymethoxy)-6-methyl-4-(trifluoromethyl)phenyl)pyridazin-3-amine as a yellow oil (100 mg, 25.0% yield) after evaporation of the combined product containing fractions under reduced pressure using a rotary evaporator. LC/MS (ESI): mass calcd for C₂₃H₃₀F₃N₅O₂: 465.24, found: m/z=466.20 [M+H]⁺.

### Step 6: Synthesis of (R)-2-(6-((1-(azetidin-3-yl)piperidin-3-yl)amino)pyridazin-3-yl)-3-methyl-5-(trifluoromethyl)phenol (88)

To a solution of (R)—N-(1-(azetidin-3-yl)piperidin-3-yl)-6-(2-(ethoxymethoxy)-6-methyl-4-(trifluoromethyl)phenyl)pyridazin-3-amine (20 mg, 0.043 mmol, 1.0 eq.) in DCM (0.5 mL) was added TFA (0.5 mL) at 0° C. (ice bath). The resulting solution was stirred at rt for 1 h followed by concentration of the reaction mixture under reduced pressure using a rotary evaporator The residue was purified by preparative RP-HPLC using the following conditions: Column: XBridge Prep OBD C18 Column, 30*150 mm, 5 μm; Mobile Phase A: Water (0.1% FA), Mobile Phase B: ACN; Flow rate: 25 mL/min; Gradient: 3% B to 17% B in 10 min; Wavelength: 254 nm; RT1: 8.05 min. After lyophilization of the combined product containing fractions, the target compound (R)-2-(6-((1-(azetidin-3-yl)piperidin-3-yl)amino)pyridazin-3-yl)-3-methyl-5-(trifluoromethyl)phenol (88) was obtained as a white solid (1.3 mg, 7.4% yield). LC/MS (ESI): mass calcd for C₂₀H₂₄F₃N₅O: 407.19, found: m/z=408.20 [M+H]⁺. ¹H NMR (400 MHz, Methanol-d₄): δ 7.36-7.33 (m, 1H), 7.10 (s, 1H), 7.03-6.98 (m, 2H), 4.18-4.01 (m, 4H), 3.50-3.43 (m, 1H), 3.34-3.33 (m, 1H), 3.15-3.10 (m, 1H), 2.69-2.66 (m, 1H), 2.21 (s, 3H), 2.13-1.86 (m, 4H), 1.78-1.72 (m, 1H), 1.59-1.46 (m, 1H) ppm. ¹⁹F NMR (282 MHz, Methanol-d₄): δ −64.42 ppm.

### Example 89: (R)-2-(6-((1-(3-Aminocyclobutyl)piperidin-3-yl)amino)-4-methylpyridazin-3-yl)-5-(trifluoromethyl)phenol (89)

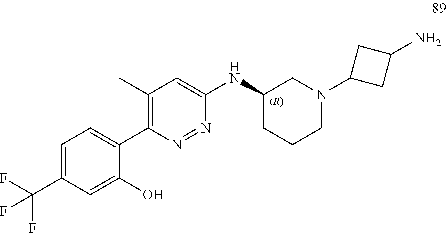

### Synthetic Route:

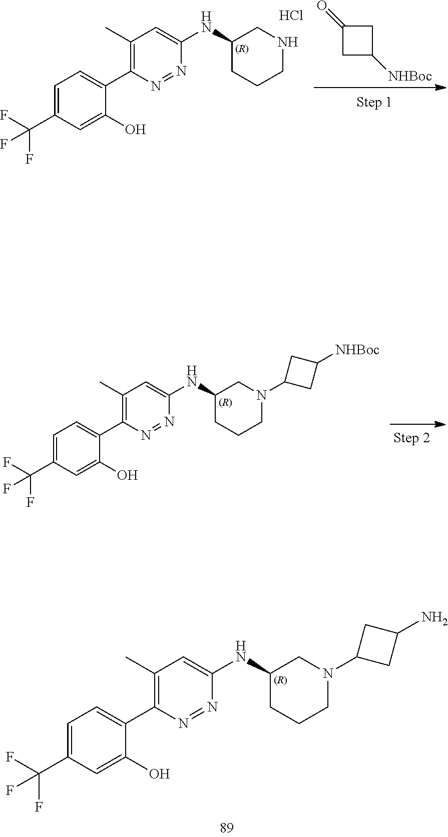

### Step 1: Synthesis of tert-butyl (R)-(3-(3-((6-(2-hydroxy-4-(trifluoromethyl)phenyl)-5-methylpyridazin-3-yl)amino)piperidin-1-yl)cyclobutyl)carbamate

To a solution of (R)-2-(4-methyl-6-(piperidin-3-ylamino)pyridazin-3-yl)-5-(trifluoromethyl)phenol (300 mg, 0.77 mmol, 1.0 eq.) and tert-butyl (3-oxocyclobutyl)carbamate (286 mg, 1.54 mmol, 2.0 eq.) in MeOH (5 mL) was added NaBH₃CN (73 mg, 1.16 mmol, 2.0 eq.). The resulting solution was stirred at rt for 2 h. The reaction mixture was concentrated under reduced pressure using a rotary evaporator. The residue was purified by silica gel column chromatography eluting with DCM/MeOH (0~20%) to afford tert-butyl (R)-(3-(3-((6-(2-hydroxy-4-(trifluoromethyl)phenyl)-5-methylpyridazin-3-yl)amino)piperidin-1-yl)cyclobutyl)carbamate as a yellow oil (350 mg, 78.3% yield) after evaporation of the combined product containing fractions under reduced pressure using a rotary evaporator. LC/MS: mass calcd for: C₂₆H₃₄F₃N₅O₃: 521.26, found: m/z=522.30 [M+H]⁺.

### Step 2: Synthesis of (R)-2-(6-((1-(3-aminocyclobutyl)piperidin-3-yl)amino)-4-methylpyridazin-3-yl)-5-(trifluoromethyl)phenol (89)

To a solution of tert-butyl (R)-(3-(3-((6-(2-hydroxy-4-(trifluoromethyl)phenyl)-5-methylpyridazin-3-yl)amino)piperidin-1-yl)cyclobutyl)carbamate (300 mg, 0.58 mmol, 1.0 eq.) in DCM (1.5 mL) was added HCl (1.5 mL, 4 M in 1,4-dioxane, 6 mmol, ~10 eq.) at 0° C. (ice bath). The resulting solution was stirred at rt for 2 h and the reaction mixture was concentrated under reduced pressure using a rotary evaporator. The residue was purified by to preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 55% B in 7 min; Wavelength: 254 nm; RT1: 6 min. After lyophilization of the combined product containing fractions, the target compound (R)-2-(6-((1-(3-aminocyclobutyl)piperidin-3-yl)amino)-4-methylpyridazin-3-yl)-5-(trifluoromethyl)phenol (89) was obtained as an off-white solid (5 mg, 2.0% yield). LC/MS: mass calcd for C₂₁H₂₆F₃N₅O: 421.21, found: m/z=422.25 [M+H]⁺. ¹H NMR (400 MHz, DMSO-d₆): δ 7.38-7.36 (m, 1H), 7.21-7.17 (m, 2H), 6.69-6.67 (m, 1H), 3.97-3.95 (m, 1H), 3.62-3.55 (m, 3H), 2.93-2.86 (m, 2H), 2.61-2.54 (m, 1H), 2.29-2.20 (m, 2H), 2.00-2.06 (m, 3H), 1.82-1.89 (m, 2H), 1.66-1.80 (m, 2H), 1.39-1.54 (m, 2H), 1.19-1.29 (m, 1H) ppm. ¹⁹F NMR (376 MHz, DMSO-d₆): δ −60.97 ppm.

### Example 90: 2-(6-(((R)-1-((1r*,3R*)-3-Aminocyclobutyl)piperidin-3-yl)amino)-4-methylpyridazin-3-yl)-5-(trifluoromethyl)phenol (90)

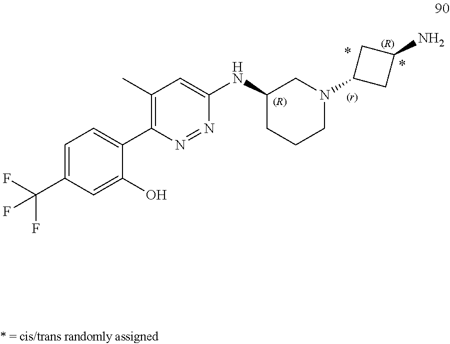

### Synthetic Route:

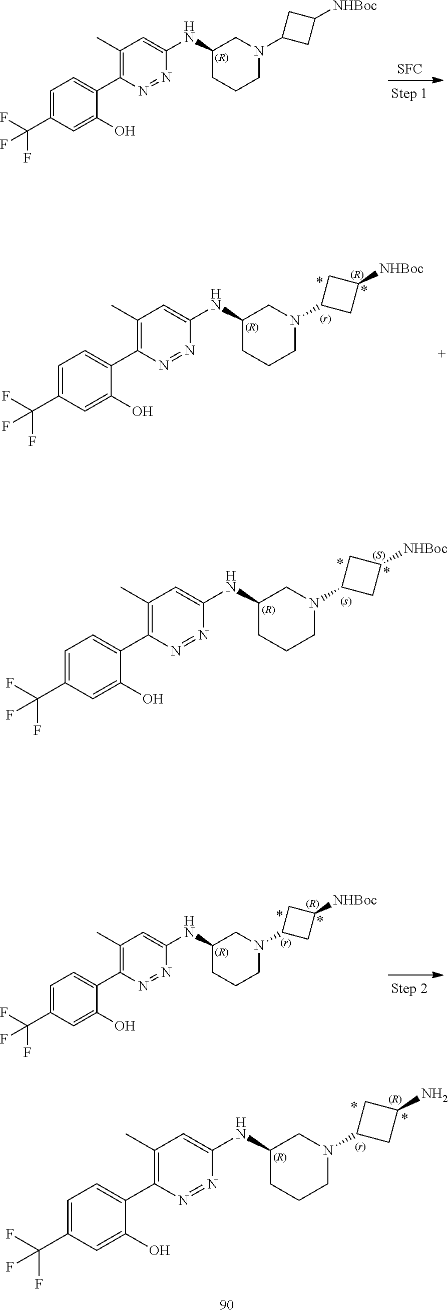

### Step 1: Synthesis of tert-butyl ((1R*,3r*)-3-((R)-3-((6-(2-hydroxy-4-(trifluoromethyl)phenyl)-5-methylpyridazin-3-yl)amino)piperidin-1-yl)cyclobutyl)carbamate

The isomers of tert-butyl (R)-(3-(3-((6-(2-hydroxy-4-(trifluoromethyl)phenyl)-5-methylpyridazin-3-yl)amino)piperidin-1-yl)cyclobutyl)carbamate (100 mg, 0.19 mmol) were separated by preparative HPLC using chiral stationary phases using the following conditions: Column: GreenSep Basic, 100*4.6 mm, 3 μm; Mobile Phase B: MeOH (1 vol-% 2M NH₃ in MeOH); Flow rate: 4 mL/min; Gradient: isocratic 5% B; Wavelength: 220 nm. After concentration of the first fraction under reduced pressure using a rotary evaporator, the first fraction, tert-butyl ((1R*,3r*)-3-((R)-3-((6-(2-hydroxy-4-(trifluoromethyl)phenyl)-5-methylpyridazin-3-yl)amino)piperidin-1-yl)cyclobutyl)carbamate, was obtained as a white solid (50 mg recovery, 100% e.e.). Isolation of the second fraction provided tert-butyl ((1S*,3s*)-3-((R)-3-((6-(2-hydroxy-4-(trifluoromethyl)phenyl)-5-methylpyridazin-3-yl)amino)piperidin-1-yl)cyclobutyl)carbamate (40 mg recovery, 100% e.e.) also as a white solid. LC/MS: mass calcd for C₂₆H₃₄F₃N₅O₃: 521.26, found: m/z=522.20 [M+H]⁺. * Cis and trans isomers were arbitrarily assigned.

### Step 2: Synthesis of 2-(6-(((R)-1-((1r*,3R*)-3-aminocyclobutyl)piperidin-3-yl)amino)-4-methylpyridazin-3-yl)-5-(trifluoromethyl)phenol (90)

To a solution of tert-butyl ((1R*,3r*)-3-((R)-3-((6-(2-hydroxy-4-(trifluoromethyl)phenyl)-5-methylpyridazin-3-yl)amino)piperidin-1-yl)cyclobutyl)carbamate (60 mg, 0.12 mmol) in DCM (2 mL) was added HCl (1 mL, 4 M in 1,4-dioxane, 4 mmol, ~35 eq.) at 0° C. (ice bath). The resulting solution was stirred at rt for overnight. The solution was concentrated under reduced pressure using a rotary evaporator to give a yellow solid which was further purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 25 mL/min; Gradient: 25% B to 55% B in 7 min, 55% B; Wavelength: 254 nm; RT1: 5 min. After lyophilization of the combined product containing fractions, the target compound 2-(6-(((R)-1-((1r*,3R*)-3-Aminocyclobutyl)piperidin-3-yl)amino)-4-methylpyridazin-3-yl)-5-(trifluoromethyl)phenol (90) was obtained as an off-white solid (34.4 mg, 70.9% yield) after lyophilization of the combined product containing fractions. LC/MS: mass calcd for C₂₁H₂₆F₃N₅O: 421.21, found: m/z=422.15 [M+H]⁺. ¹H NMR (400 MHz, Methanol-d₄): δ 7.63-7.33 (m, 1H), 7.28-7.10 (m, 2H), 6.75 (s, 1H), 4.26-3.93 (m, 1H), 3.19-2.97 (m, 2H), 2.76-2.62 (m, 1H), 2.55-2.36 (m, 3H), 2.17 (s, 3H), 2.10-1.97 (m, 2H), 1.94-1.76 (m, 2H), 1.73-1.57 (m, 3H), 1.51-1.23 (m, 1H) ppm. ¹⁹F NMR (386 MHz, Methanol-d₄): δ −64.28 ppm.

### Example 91: 2-(6-(((R)-1-((1s*,3S*)-3-aminocyclobutyl)piperidin-3-yl)amino)-4-methylpyridazin-3-yl)-5-(trifluoromethyl)phenol (91)

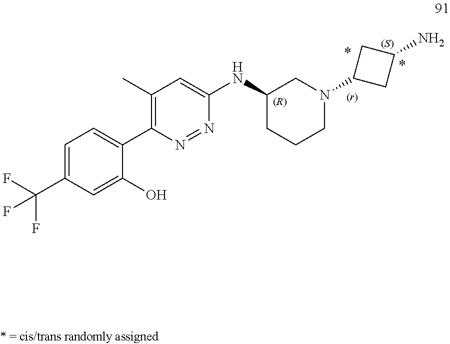

### Synthetic Route:

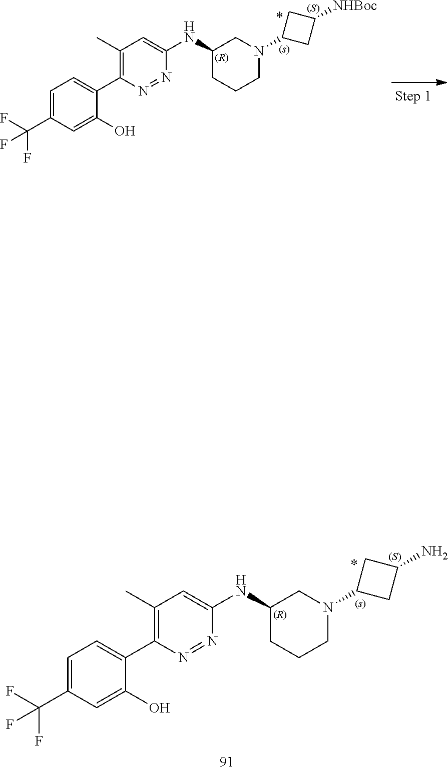

### Step 1: Synthesis of 2-(6-(((R)-1-((1s*,3S*)-3-aminocyclobutyl)piperidin-3-yl)amino)-4-methylpyridazin-3-yl)-5-(trifluoromethyl)phenol (91)

To a solution of tert-butyl ((1S*,3s*)-3-((R)-3-((6-(2-hydroxy-4-(trifluoromethyl)phenyl)-5-methylpyridazin-3-yl)amino)piperidin-1-yl)cyclobutyl)carbamate (10 mg, 0.02 mmol) in DCM (0.2 mL) was added HCl (0.25 mL, 4 M in 1,4-dioxane, 1 mmol, ~50 eq.) at 0° C. (ice bath). The resulting solution was stirred at rt for overnight. The solution was concentrated under reduced pressure using a rotary evaporator to afford a yellow solid which was further purified by preparative RP-HPLC using following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 25 mL/min; Gradient: 25% B to 55% B in 7 min, 55% B; Wavelength: 254 nm; RT1: 5 min. After lyophilization of the combined product containing fractions, the target compound 2-(6-(((R)-1-((1s*,3S*)-3-aminocyclobutyl)piperidin-3-yl)amino)-4-methylpyridazin-3-yl)-5-(trifluoromethyl)phenol (91) was obtained as an off-white solid (4.0 mg, 49.3% yield). LC/MS: mass calcd for C₂₁H₂₆F₃N₅O: 421.21, found: m/z=422.15 [M+H]⁺. ¹H NMR (400 MHz, Methanol-d₄): δ 7.57-7.32 (m, 1H), 7.27-7.05 (m, 2H), 6.92 (s, 1H), 4.45-3.99 (m, 1H), 3.62-3.50 (m, 1H) 3.23-3.01 (m, 2H), 2.80-2.64 (m, 1H), 2.40-2.24 (m, 2H), 2.12 (s, 3H), 2.10-1.81 (m, 6H), 1.80-1.60 (m, 1H), 1.57-1.33 (m, 1H) ppm. ¹⁹F NMR (386 MHz, Methanol-d₄): δ −64.28 ppm.

### Example 92: (R)-2-Amino-1-(3-((6-(2-hydroxy-4-(trifluoromethyl)phenyl)-5-methylpyridazin-3-yl)amino)piperidin-1-yl)ethan-1-one (92)

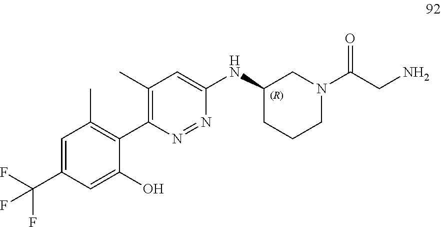

### Synthetic Route:

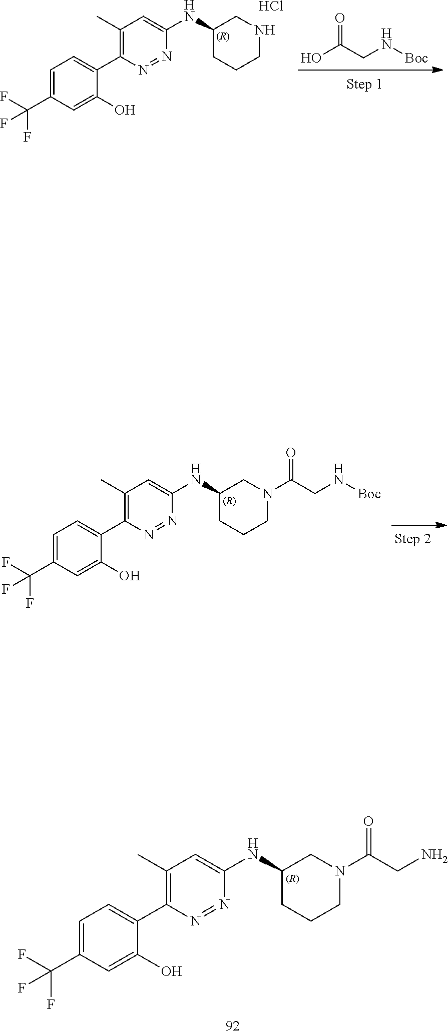

### Step 1: Synthesis of tert-butyl (R)-(2-(3-((6-(2-hydroxy-4-(trifluoromethyl)phenyl)-5-methylpyridazin-3-yl)amino)piperidin-1-yl)-2-oxoethyl)carbamate

A brown solution of (R)-2-(4-methyl-6-(piperidin-3-ylamino)pyridazin-3-yl)-5-(trifluoromethyl)phenol (200 mg, 0.51 mmol, 1.0 eq.), (tert-butoxycarbonyl)glycine (108 mg, 0.62 mmol, 1.2 eq.), BOP (341 mg, 0.77 mmol, 1.5 eq.) and DIEA (268 μL, 199 mg, 1.54 mmol, 3.0 eq.) in DMF (2 mL) was stirred at rt for 3 h. To this solution, EtOAc (10 mL) and water (10 mL) were added. After separation of phases, the aqueous phase was extracted with EtOAc (2×10 mL). The combined organic phases were washed with brine (3×30 mL), dried with anhydrous Na₂SO₄, filtered, and the filtrate was concentrated under reduced pressure using a rotary evaporator to afford tert-butyl (R)-(2-(3-((6-(2-hydroxy-4-(trifluoromethyl)phenyl)-5-methylpyridazin-3-yl)amino)piperidin-1-yl)-2-oxoethyl)carbamate as a brown oil (120 mg, 45.8% yield). LC/MS: mass calcd for C₂₄H₃₀F₃N₅O₄: 509.22, found: m/z=510.20 [M+H]⁺.

### Step 2: Synthesis of (R)-2-amino-1-(3-((6-(2-hydroxy-4-(trifluoromethyl)phenyl)-5-methylpyridazin-3-yl)amino)piperidin-1-yl)ethan-1-one (92)

HCl (3 mL, 4 M in 1,4-dioxane) was added to a brown solution of tert-butyl (R)-(2-(3-((6-(2-hydroxy-4-(trifluoromethyl)phenyl)-5-methylpyridazin-3-yl)amino)piperidin-1-yl)-2-oxoethyl)carbamate (110 mg, 0.22 mmol, 1.0 eq.) in DCM (3 mL) was added HCl (3 mL, 4 M solution in 1,4-dioxane, 12 mmol, ~56 eq.) resulting in a suspension, which was stirred at rt for 3 h. After removal of solvents under reduced pressure using a rotary evaporator, the residue was purified by preparative RP-HPLC using the following conditions Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 25 mL/min; Gradient: 25% B to 55% B in 7 min, 55% B; Wavelength: 220 nm; RT1: 5 min to afford the target compound (R)-2-amino-1-(3-((6-(2-hydroxy-4-(trifluoromethyl)phenyl)-5-methylpyridazin-3-yl)amino)piperidin-1-yl)ethan-1-one (92) as a white solid (15 mg, 16.6% yield) after lyophilization of the combined product containing fractions. LC/MS: mass calcd for C₁₉H₂₂F₃N₅O₂: 409.17, found: m/z=410.15 [M+H]⁺. ¹H NMR (400 MHz, Methanol-d₄): δ 7.44-7.37 (m, 1H), 7.29-7.11 (m, 2H), 6.86-6.79 (m, 1H), 4.33-3.88 (m, 3H), 3.73-3.46 (m, 1H), 3.31-3.15 (m, 2H), 2.92-2.51 (m, 1H), 2.23-2.04 (m, 4H), 1.95-1.84 (m, 1H), 1.81-1.52 (m, 2H) ppm. ¹⁹F NMR (376 MHz, Methanol-d₄): δ −64.27 ppm.

### Example 93: (R)-2-(3-((6-(2-Hydroxy-4-(trifluoromethyl)phenyl)-5-methylpyridazin-3-yl)amino)piperidin-1-yl)-1-(3,9-diazaspiro[5.5]undecan-3-yl)ethan-1-one (93)

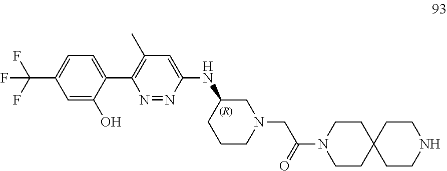

### Synthetic Route:

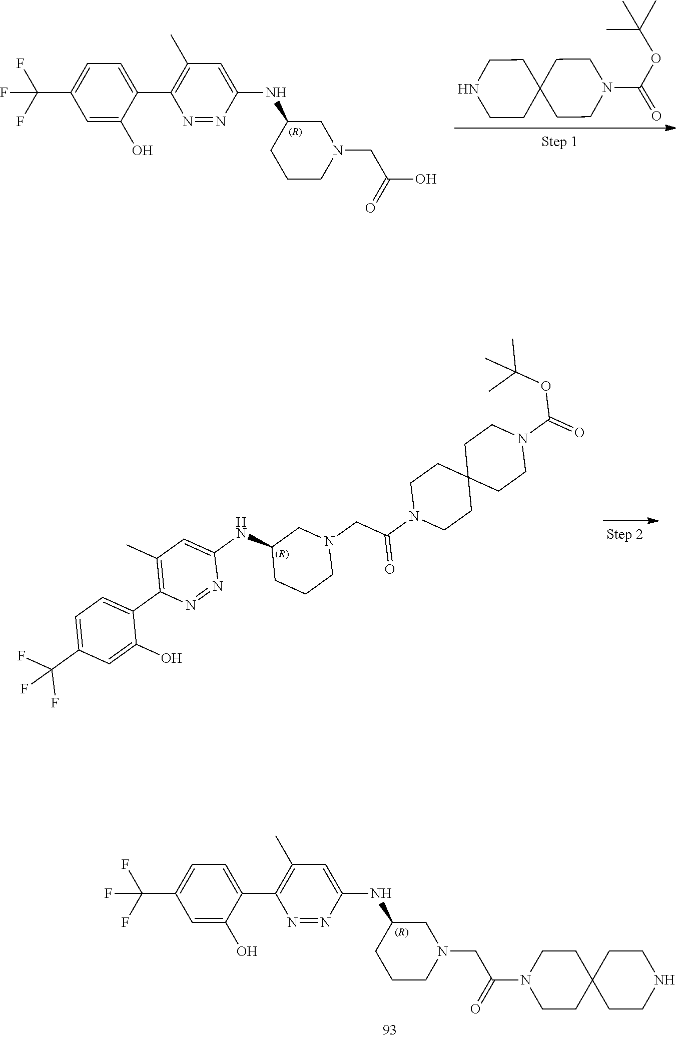

### Step 1: Synthesis of tert-butyl (R)-9-(2-(3-((6-(2-hydroxy-4-(trifluoromethyl)phenyl)-5-methylpyridazin-3-yl)amino)piperidin-1-yl)acetyl)-3,9-diazaspiro[5.5]undecane-3-carboxylate

A solution of (R)-2-(3-((6-(2-hydroxy-4-(trifluoromethyl)phenyl)-5-methylpyridazin-3-yl)amino)piperidin-1-yl)acetic acid (200 mg, 0.5 mmol, 1.0 eq.) and tert-butyl 3,9-diazaspiro[5.5]undecane-3-carboxylate (190 mg, 0.7 mmol, 1.5 eq.) in DMF (5 mL) was treated with DIEA (240 μL, 178 mg, 1.4 mmol, 2.8 eq.), and HOBt (66 mg, 0.5 mmol, 1.0 eq.) at 0° C. (ice bath) under an atmosphere of N₂ followed by the addition of EDCI (140 mg, 0.7 mmol, 1.5 eq.) in portions at 0° C. (ice bath). The reaction mixture was stirred for overnight with warming to rt, followed by quenching with a ice/water mixture (10 mL). The mixture was extracted with EA (3×10 mL), and the combined organic extracts were washed with H₂O (3×10 mL) and dried over anhydrous Na₂SO₄. After filtration, the filtrate was concentrated under reduced pressure using a rotary evaporator. The residue was purified by silica gel column chromatography eluting with MeOH/DCM gradients (0~9%) to afford tert-butyl (R)-9-(2-(3-((6-(2-hydroxy-4-(trifluoromethyl)phenyl)-5-methylpyridazin-3-yl)amino)piperidin-1-yl)acetyl)-3,9-diazaspiro[5.5]undecane-3-carboxylate (190 mg, 60.2% yield) as a yellow solid after evaporation of the combined product containing fractions under reduced pressure using a rotary evaporator. LC/MS (ESI): mass calcd for C₃₃H₄₅F₃N₆O₄: 646.35, found: m/z=647.50 [M+H]⁺.

### Step 2: Synthesis of (R)-2-(3-((6-(2-hydroxy-4-(trifluoromethyl)phenyl)-5-methylpyridazin-3-yl)amino)piperidin-1-yl)-1-(3,9-diazaspiro[5.5]undecan-3-yl)ethan-1-one (93)

To a stirred mixture of tert-butyl (R)-9-(2-(3-((6-(2-hydroxy-4-(trifluoromethyl)phenyl)-5-methylpyridazin-3-yl)amino)piperidin-1-yl)acetyl)-3,9-diazaspiro[5.5]undecane-3-carboxylate (150 mg, 0.2 mmol, 1.0 eq.) in THF (4 mL) was added HCl (2 mL, 4 M in 1,4-dioxane, 8 mmol, 40 eq.) at 0° C. (ice bath). The resulting mixture was stirred for 3 h at rt followed by concentration under reduced pressure using a rotary evaporator. The crude product (140 mg) was purified preparative RP-HPLC using the following conditions: Column: XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min mL/min; Gradient: 19% B to 35% B in 10 min; Wavelength: 254 nm/220 nm; RT1: 8.38 min) to afford the target compound (R)-2-(3-((6-(2-hydroxy-4-(trifluoromethyl)phenyl)-5-methylpyridazin-3-yl)amino)piperidin-1-yl)-1-(3,9-diazaspiro[5.5]undecan-3-yl)ethan-1-one (20.4 mg, 15.9%) (93) as a white solid after lyophilization of the combined product containing fractions. LC/MS (ESI): mass calcd for C₂₈H₃₇F₃N₆O₂: 546.29, found: m/z=547.25 [M+H]⁺. ¹H NMR (300 MHz, Methanol-d₄): δ 7.36 (d, J=7.8 Hz, 1H), 7.20-7.10 (m, 2H), 6.76 (s, 1H), 4.20-4.08 (m, 1H), 3.70-3.60 (m, 2H), 3.48-2.58 (m, 2H), 3.25-3.20 (m, 2H), 3.00-2.80 (m, 5H), 2.65-2.50 (m, 1H), 2.40-2.20 (m, 2H), 2.13 (s, 3H), 2.00-1.75 (m, 2H), 1.70-1.50 (m, 10H) ppm. ¹⁹F NMR (282 MHz, Methanol-d₄): δ −64.21 ppm.

### Example 94: (R)—N-(Azetidin-3-yl)-2-(3-((6-(2-hydroxy-4-(trifluoromethyl)phenyl)-5-methylpyridazin-3-yl)amino)piperidin-1-yl)acetamide (94)

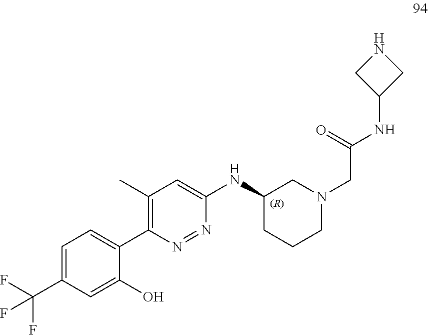

### Synthetic Route:

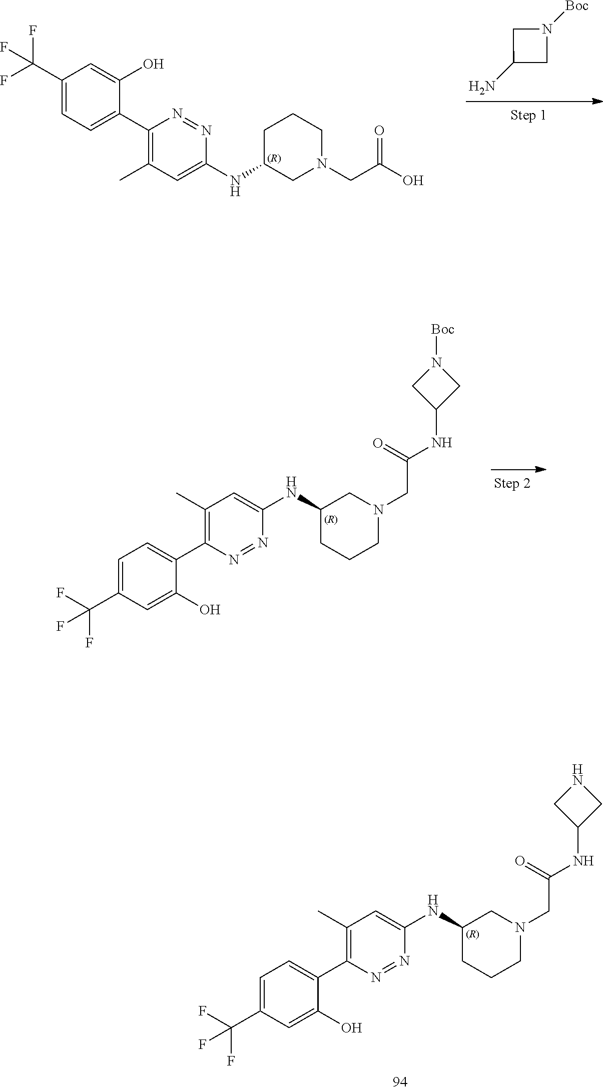

### Step 1: Synthesis of tert-butyl (R)-3-(2-(3-((6-(2-hydroxy-4-(trifluoromethyl)phenyl)-5-methylpyridazin-3-yl)amino)piperidin-1-yl) acetamido) azetidine-1-carboxylate

To a yellow solution of (R)-2-(3-((6-(2-hydroxy-4-(trifluoromethyl)phenyl)-5-methylpyridazin-3-yl)amino)piperidin-1-yl)acetic acid (100 mg, 0.24 mmol, 1.0 eq.) and tert-butyl 3-aminoazetidine-1-carboxylate (51 mg, 0.29 mmol, 1.2 eq.) in DMF (3 mL), DIEA (170 μL, 126 mg, 0.98 mmol, 4.0 eq.) was added affording a solution to which was added BOP (129 mg, 0.29 mmol, 1.2 eq.). The solution was stirred for 4 h at rt. To this solution, EtOAc (10 mL) and water (10 mL) were added. After separation of phases, the aqueous phase was extracted with EtOAc (2×10 mL). The combined organic phases were washed with brine (3×30 mL), dried with anhydrous Na₂SO₄, filtered, and the filtrate was concentrated under reduced pressure using a rotary evaporator to afford tert-butyl (R)-3-(2-(3-((6-(2-hydroxy-4-(trifluoromethyl)phenyl)-5-methylpyridazin-3-yl)amino)piperidin-1-yl) acetamido) azetidine-1-carboxylate as a yellow solid (50 mg, 33.9 yield) which was used directly without further isolation, purification, and characterization in the next step. LC/MS: mass calcd for C₂₇H₃₅F₃N₆O₄: 564.27, found: m/z=565.35 [M+H]⁺.

### Step 2: Synthesis of (R)—N-(azetidin-3-yl)-2-(3-((6-(2-hydroxy-4-(trifluoromethyl)phenyl)-5-methylpyridazin-3-yl)amino)piperidin-1-yl)acetamide (18)

To a yellow solution of tert-butyl (R)-3-(2-(3-((6-(2-hydroxy-4-(trifluoromethyl)phenyl)-5-methylpyridazin-3-yl)amino)piperidin-1-yl) acetamido) azetidine-1-carboxylate (50 mg, 0.09 mmol, 1.0 eq.) in DCM (2 mL), HCl (1 mL, 4 M in 1,4-dioxane, 4 mmol, ~45 eq.) was added affording a suspension. The suspension was stirred for 4 h at rt. Volatiles were removed under reduced pressure using a rotary evaporator affording a yellow solid which was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 47% B in 7 min, 47% B; Wavelength: 254 nm; RT1: 5.58 min. After lyophilization of the combined product containing fractions, the target compound (R)—N-(azetidin-3-yl)-2-(3-((6-(2-hydroxy-4-(trifluoromethyl)phenyl)-5-methylpyridazin-3-yl)amino)piperidin-1-yl)acetamide (94) (12.0 mg, 29.0% yield was obtained as an off-white solid. LC/MS: mass calcd for C₂₂H₂₇F₃N₆O₂: 464.21, found: m/z=465.30 [M+H]⁺. ¹H NMR (400 MHz, Methanol-d₄): δ 7.41-7.35 (m, 1H), 7.22-7.13 (m, 3H), 6.84 (s, 1H), 4.77-4.69 (m, 1H), 4.24-4.19 (m, 2H), 3.93-3.73 (m, 3H), 3.18-2.95 (m, 3H), 2.73-2.43 (m, 2H), 2.40-2.21 (m, 1H), 2.20-2.14 (m, 3H), 1.99-1.85 (m, 2H), 1.84-1.68 (m, 1H), 1.66-1.48 (m, 1H), 1.39-1.17 (m, 1H) ppm. ¹⁹F NMR (376 MHz, Methanol-d₄): δ −64.29 ppm.

### Example 95: (R)-2-(3-((6-(2-Hydroxy-4-(trifluoromethyl)phenyl)-5-methylpyridazin-3-yl)amino)piperidin-1-yl)-N-(piperidin-4-yl)acetamide (95)

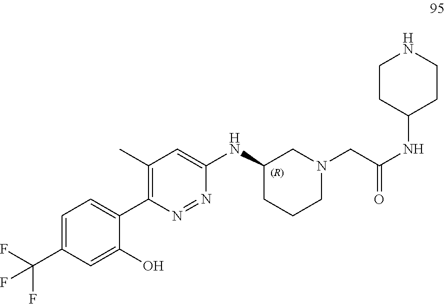

### Synthetic Route:

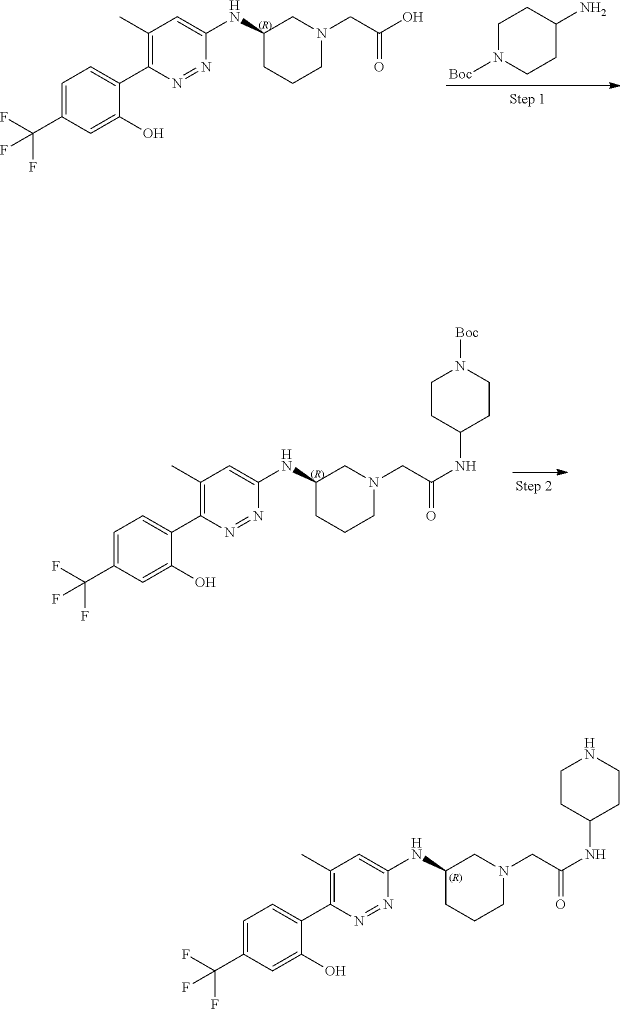

### Step 1: Synthesis of tert-butyl (R)-4-(2-(3-((6-(2-hydroxy-4-(trifluoromethyl)phenyl)-5-methylpyridazin-3-yl)amino)piperidin-1-yl) acetamido) piperidine-1-carboxylate

To a yellow solution of (R)-2-(3-((6-(2-hydroxy-4-(trifluoromethyl)phenyl)-5-methylpyridazin-3-yl)amino)piperidin-1-yl)acetic acid (100 mg, 0.24 mmol, 1.0 eq.), tert-butyl 4-aminopiperidine-1-carboxylate (59 mg, 0.29 mmol, 1.2 eq.), EDCI (70 mg, 0.37 mmol, 1.5 eq.), HOBt (49 mg, 0.37 mmol, 1.5 eq.), and DIEA (94 μL, 70 mg, 0.37 mmol, 1.5 eq.) in DMF (1.5 mL) was stirred for 1 h at rt. To this reaction mixture, EtOAc (20 mL) and water (10 mL) were added. The aqueous phases were extracted with EtOAc (2×10 mL). The combined organic phases were washed with brine (2×50 mL), dried with anhydrous Na₂SO₄, filtered, and the filtrate was concentrated under reduced pressure using a rotary evaporator to afford tert-butyl (R)-4-(2-(3-((6-(2-hydroxy-4-(trifluoromethyl)phenyl)-5-methylpyridazin-3-yl)amino)piperidin-1-yl) acetamido) piperidine-1-carboxylate as brown-yellow oil (115 mg, 68.7% yield) which was used directly without further isolation, purification and characterization in the next step. LC/MS: mass calcd for C₂₉H₃₉F₃N₆O₄: 592.30, found: m/z=593.00 [M+H]⁺.

### Step 2: Synthesis of (R)-2-(3-((6-(2-yydroxy-4-(trifluoromethyl)phenyl)-5-methylpyridazin-3-yl)amino)piperidin-1-yl)-N-(piperidin-4-yl)acetamide (95)

To a solution of tert-butyl (R)-4-(2-(3-((6-(2-hydroxy-4-(trifluoromethyl)phenyl)-5-methylpyridazin-3-yl)amino)piperidin-1-yl) acetamido) piperidine-1-carboxylate (115 mg, 0.19 mmol) in DCM (3 mL) was added HCl (1.5 mL, 4 M in 1,4-dioxane, 6 mmol, ~31 eq.) at 0° C. (ice bath). The resulting solution was stirred for overnight at rt followed by concentration under reduced pressure using a rotary evaporator to yield a yellow solid which was purified by preparative RP-HPLC using the following conditions: Column: XBridge Shield RP18 OBD Column, 30*150 mm, 5 μm; Mobile Phase A: Water (10 mmol/L NH₄HCO₃+0.1% NH₃), Mobile Phase B: ACN; Flow rate: 60 mL/min; Gradient: 30% B to 55% B in 7 min, 55% B; Wavelength: 254 nm; RT1: 6.32 min. After lyophilization of the combined product containing fractions, the target compound (R)-2-(3-((6-(2-yydroxy-4-(trifluoromethyl)phenyl)-5-methylpyridazin-3-yl)amino)piperidin-1-yl)-N-(piperidin-4-yl)acetamide (95) format salt was obtained as an off-white solid (12.8 mg, 11.7% yield). LC/MS: mass calcd for C₂₅H₃₃F₃N₆O₄: 492.25, found: m/z=493.30 [M+H]⁺. ¹H NMR (300 MHz, Methanol-d₄): δ 7.44-7.35 (m, 1H), 7.26-7.15 (m, 2H), 6.85-6.79 (m, 1H), 4.23-4.17 (m, 1H), 3.96-3.83 (m, 1H), 3.26-2.76 (m, 6H), 2.70-2.64 (m, 1H), 2.48-2.42 (m, 1H), 2.19-2.11 (m, 4H), 1.59-1.89 (m, 4H), 1.80-1.74 (m, 4H), 1.59-1.53 (m, 1H) ppm. ¹⁹F NMR (282 MHz, Methanol-d₄): δ −64.30 ppm.

### Example 96: (R)-3-Amino-2,2-difluoro-1-(3-((6-(2-hydroxy-4-(trifluoromethyl)phenyl)-5-methylpyridazin-3-yl)amino)piperidin-1-yl) propan-1-one (96)

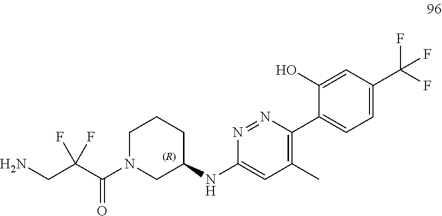

### Synthetic Route:

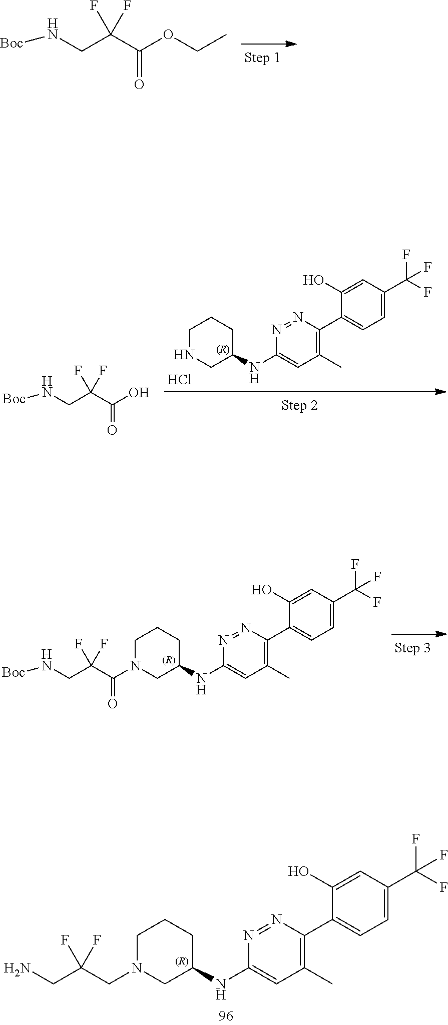

### Step 1:3-((tert-butoxycarbonyl)amino)-2,2-difluoropropanoic Acid

To a solution of ethyl 3-((tert-butoxycarbonyl)amino)-2,2-difluoropropanoate (1.00 g, 3.95 mmol, 1.0 eq.) in EtOH/H₂O (10 mL/3 mL) was added LiOH (0.19 g, 7.90 mmol, 2.0 eq.) at 0° C. (ice bath). The resulting solution was stirred for overnight at rt. The solution was frozen and lyophilized to afford lithium 3-((tert-butoxycarbonyl)amino)-2,2-difluoropropanoate as a white solid (700 mg, 69.3% yield). LC/MS: mass calcd for C₈H₁₃F₂NO₄: 225.08, found: 224.00 [M−H]⁻.

### Step 2: Synthesis of tert-butyl (R)-(2,2-difluoro-3-(3-((6-(2-hydroxy-4-(trifluoromethyl)phenyl)-5-methylpyridazin-3-yl)amino)piperidin-1-yl)-3-oxopropyl)carbamate

EDCI (766 mg, 4.00 mmol, 1.5 eq.) and HOBT (540 mg, 4.00 mmol, 1.5 eq.) were added to a colorless solution of lithium 3-((tert-butoxycarbonyl)amino)-2,2-difluoropropanoate (600 mg, 2.66 mmol, 1.0 eq.), (R)-2-(4-methyl-6-(piperidin-3-ylamino)pyridazin-3-yl)-5-(trifluoromethyl)phenol hydrochloride (1.01 g, 2.66 mmol, 1.0 eq.), and DIEA (1.39 mL, 1.03 g, 7.99 mmol, 3.0 eq.) in DMF (5 mL) affording a slight yellow solution which was stirred for overnight at rt. To this reaction mixture, EtOAc (20 mL) and water (10 mL) were added. The aqueous phase was extracted with EtOAc (2×10 mL) and the combined organic phases were washed with brine (2×50 mL), dried with anhydrous Na₂SO₄, filtered, and the filtrate concentrated under reduced pressure using a rotary evaporator. The residue was purified by silica gel column chromatography to afford tert-butyl (R)-(2,2-difluoro-3-(3-((6-(2-hydroxy-4-(trifluoromethyl)phenyl)-5-methylpyridazin-3-yl)amino)piperidin-1-yl)-3-oxopropyl)carbamate as a white solid (400 mg, 24.5% yield) after evaporation of the combined product containing fractions under reduced pressure using a rotary evaporator. LC/MS: mass calcd for C₂₅H₃₀F₅N₅O₄: 559.22, found: m/z=560.00 [M+H]⁺.

### Step 3: Synthesis of (R)-3-amino-2,2-difluoro-1-(3-((6-(2-hydroxy-4-(trifluoromethyl)phenyl)-5-methylpyridazin-3-yl)amino)piperidin-1-yl) propan-1-one (96)

To a solution of tert-butyl (R)-(2,2-difluoro-3-(3-((6-(2-hydroxy-4-(trifluoromethyl)phenyl)-5-methylpyridazin-3-yl)amino)piperidin-1-yl)-3-oxopropyl)carbamate (50 mg, 0.09 mmol, 1.0 eq.) in DCM (1 mL) was added HCl (0.5 mL, 4 M in 1,4-dioxane, 2 mmol, ~23 eq.) at 0° C. (ice bath). The resulting solution was stirred for overnight with warming to rt followed by concentration under reduced pressure using a rotary evaporator to afford a yellow solid which was purified by preparative RP-using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min; Gradient: 27% B to 57% B in 7 min, 57% B; Wavelength: 254 nm; RT1: 5.23 min. After lyophilization of the combined product containing fractions, the target compound (R)-3-amino-2,2-difluoro-1-(3-((6-(2-hydroxy-4-(trifluoromethyl)phenyl)-5-methylpyridazin-3-yl)amino)piperidin-1-yl) propan-1-one (96) as an off-white solid (21.4 mg, 52.1% yield). LC/MS: mass calcd for C₂₀H₂₂F₅N₅O₂: 459.17, found: m/z=460.15 [M+H]⁺. ¹H NMR (300 MHz, Methanol-d₄): δ 7.46-7.38 (m, 1H), 7.28-7.22 (m, 1H), 7.20-7.15 (m, 1H), 6.82 (s, 1H), 4.33-4.23 (m, 1H), 4.21-4.09 (m, 1H), 4.07-3.97 (m, 1H), 3.59-3.38 (m, 1H), 3.36-3.15 (m, 3H), 2.16 (s, 4H), 1.97-1.91 (m, 1H), 1.83-1.65 (m, 2H) ppm. ¹⁹F NMR (300 MHz, Methanol-d₄): δ −64.29, −107.62 ppm.

### Example 97: (R)-2-(6-((1-(3-Amino-2,2-difluoropropyl)piperidin-3-yl)amino)-4-methylpyridazin-3-yl)-5-(trifluoromethyl)phenol (97)

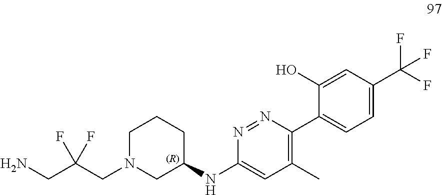

### Synthetic Route:

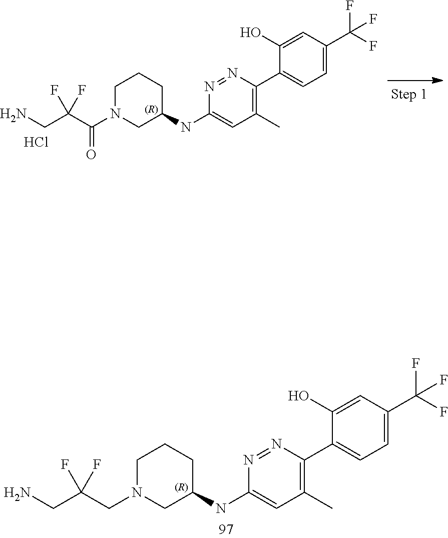

### Step 1: Synthesis of (R)-2-(6-((1-(3-amino-2,2-difluoropropyl)piperidin-3-yl)amino)-4-methylpyridazin-3-yl)-5-(trifluoromethyl)phenol (97)

Borane dimethyl sulfide complex (BH₃·Me₂S) (0.12 mL, 1.20 mmol, 10 M (>95%), 3.0 eq.) was added into a solution of (R)-3-amino-2,2-difluoro-1-(3-((6-(2-hydroxy-4-(trifluoromethyl)phenyl)-5-methylpyridazin-3-yl)amino)piperidin-1-yl) propan-1-one hydrochloride (200 mg, 0.40 mmol, 1.0 eq.) in THF (5 mL) at 0° C. (ice bath), and the obtained yellow solution was stirred for overnight at 60° C. After cooling to rt, MeOH was added at 0° C. (ice bath) to quench the reaction. After concentration under reduced pressure using a rotary evaporator, the residue was purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min; Gradient: 30% B to 60% B in 7 min, 60% B; Wavelength: 254 nm; RT1: 6 min. After lyophilization of the combined product containing fractions, the target compound (R)-2-(6-((1-(3-amino-2,2-difluoropropyl)piperidin-3-yl)amino)-4-methylpyridazin-3-yl)-5-(trifluoromethyl)phenol (97) was obtained as a white solid (22.3 mg, 12.4% yield). LC/MS: mass calcd for C₂₀H₂₄F₅N₅O: 445.19, found: m/z=446.2 [M+H]⁺. ¹H NMR (400 MHz, Methanol-d₄): δ 7.42 (d, J=7.9 Hz, 1H), 7.24 (d, J=7.9 Hz, 1H), 7.18 (s, 1H), 6.81 (s, 1H), 4.17-4.08 (m, 1H), 3.17-3.02 (m, 3H), 2.95-2.74 (m, 3H), 2.38-2.38 (m, 2H), 2.15 (s, 3H), 2.00-1.92 (m, 1H), 1.90-1.78 (m, 1H), 1.75-1.64 (m, 1H), 1.51-1.41 (m, 1H) ppm.

### Example 98: (R)-2-(6-((1′-(2-Aminoethyl)-[1,4′-bipiperidin]-3-yl)amino)-4-methylpyridazin-3-yl)-5-(trifluoromethyl)phenol (98)

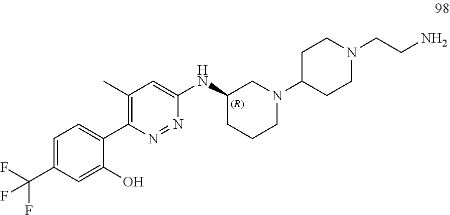

### Synthetic Route:

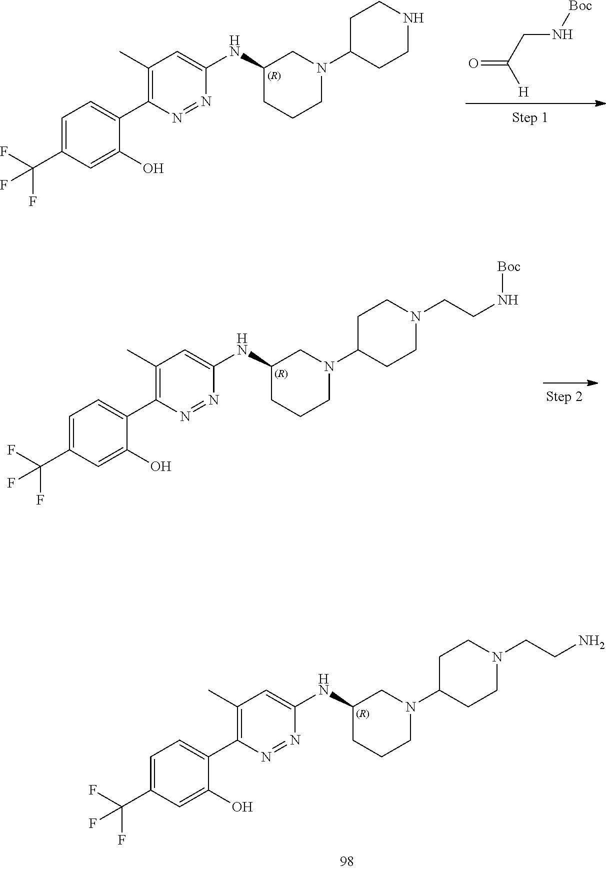

### Step 1: Synthesis of tert-butyl (R)-(2-(3-((6-(2-hydroxy-4-(trifluoromethyl)phenyl)-5-methylpyridazin-3-yl)amino)-[1,4′-bipiperidin]-1′-yl)ethyl)carbamate

A colorless suspension of (R)-2-(6-([1,4′-bipiperidin]-3-ylamino)-4-methylpyridazin-3-yl)-5-(trifluoromethyl)phenol (300 mg, 0.69 mmol, 1.0 eq.) and tert-butyl (2-oxoethyl)carbamate (219 mg, 1.38 mmol, 2.0 eq.) in MeOH (6 mL) was stirred for 0.5 h at rt. To this suspension, NaBH₃CN (87 mg, 1.38 mmol, 2.0 eq.) was added in small portions leading to evolution of hydrogen gas and resulting in a colorless solution, which was stirred for overnight at rt. The reaction was quenched by addition of water (5 mL). The obtained mixture was extracted with EtOAc (2×20 mL) and the combined organic phases were washed with brine (2×10 mL), dried with anhydrous Na₂SO₄, filtered, and the filtrate was concentrated under reduced pressure using a rotary evaporator. The residue was purified by silica gel column chromatography to afford tert-butyl (R)-(2-(3-((6-(2-hydroxy-4-(trifluoromethyl)phenyl)-5-methylpyridazin-3-yl)amino)-[1,4′-bipiperidin]-1′-yl)ethyl)carbamate as yellow oil (270 mg, 57.1% yield). LC/MS: mass calcd for C₂₉H₄₁F₃N₆O₃: 578.32, found: m/z=579.35 [M+H]⁺.

### Step 2: Synthesis of (R)-2-(6-((1′-(2-aminoethyl)-[1,4′-bipiperidin]-3-yl)amino)-4-methylpyridazin-3-yl)-5-(trifluoromethyl)phenol (98)

To a solution of tert-butyl (R)-(2-(3-((6-(2-hydroxy-4-(trifluoromethyl)phenyl)-5-methylpyridazin-3-yl)amino)-[1,4′-bipiperidin]-1′-yl)ethyl)carbamate (90 mg, 0.16 mmol, 1.0 eq.) in DCM (2 ml) was added HCl (1 ml, 4 M in 1,4-dioxane, 4 mmol, ~25 eq.). The reaction was stirred for 3 h at rt. After concentration under reduced pressure using a rotary evaporator, the residue was purified by preparative RP-HPLC using the following conditions: Column: Atlantis Prep T3 OBD Column, 19*250 mm 10 μm; Mobile Phase A: Water (0.1% FA), Mobile Phase B: ACN (0.1% FA); Flow rate: 25 mL/min; Gradient up to 37% B in 10 min; Wavelength: 254 nm; RT1: 7.38 min. After lyophilization of the combined product containing fractions, the target compound (R)-2-(6-((1′-(2-aminoethyl)-[1,4′-bipiperidin]-3-yl)amino)-4-methylpyridazin-3-yl)-5-(trifluoromethyl)phenol (98) format salt was obtained as a light yellow solid (19.8 mg, 24.2% yield). LC/MS: mass calcd for C₂₅H₃₅F₃N₆O₃: 478.27, found: m/z=479.2 [M+H]⁺. ¹H NMR (300 MHz, Methanol-d₄): δ 7.34-7.43 (m, 1H), 7.28-7.20 (m, 1H), 7.18 (s, 1H), 6.81 (s, 1H), 4.31-4.20 (m, 1H), 3.66-3.56 (m, 1H), 3.29-3.19 (m, 1H), 3.11-2.98 (m, 4H), 2.97-2.74 (m, 2H), 2.73-2.55 (m, 3H), 2.21-1.95 (m, 8H), 1.92-1.54 (m, 4H) ppm. ¹⁹F NMR (282 MHz, Methanol-d₄): δ −64.32 ppm.

### Example 99: (R)-2-(4-((1-(Azetidin-3-yl)piperidin-3-yl)amino)-5,7-dihydrofuro[3,4-d]pyridazin-1-yl)-5-(trifluoromethyl)phenol (99)

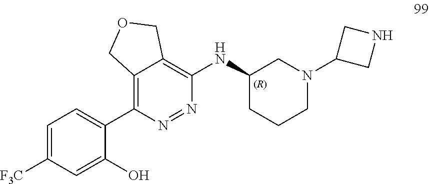

### Synthetic Route:

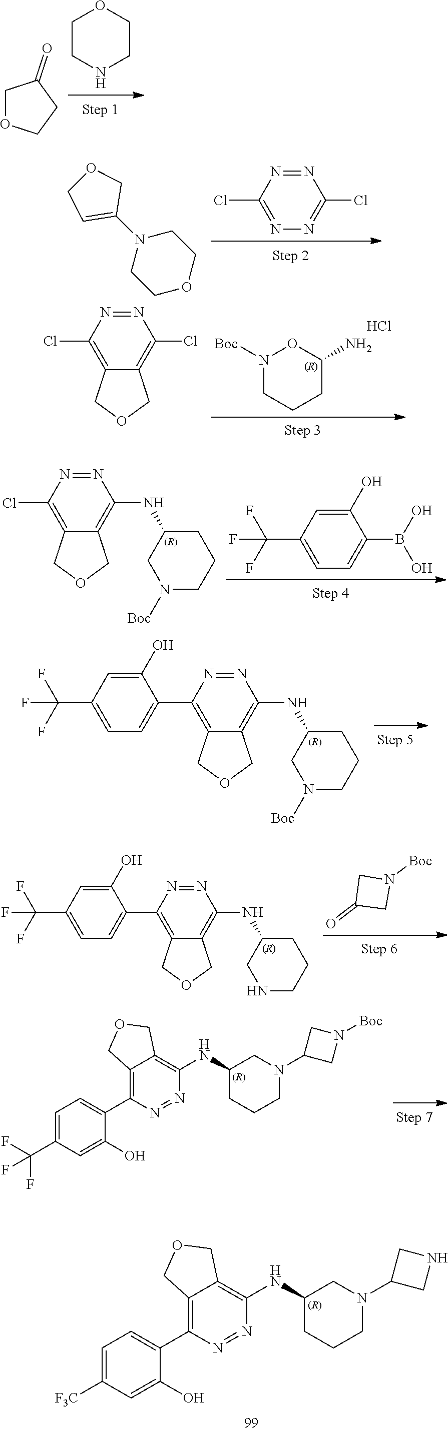

### Step 1: Synthesis of 4-(2,5-dihydrofuran-3-yl)morpholine

To a 200 mL sealed tube equipped with a magnetic stirring bar were added dihydrofuran-3 (2H)-one (6.21 mL, 6.91 g, 80.3 mmol, 1.0 eq.), morpholine (4.70 mL, 4.75 g, 54.0 mmol, 2.0 eq.), anhydrous MgSO₄ (30.0 g, 249 mmol, 3.1 eq.) in Et₂O (150 mL). The resulting solution was stirred for 48 h at rt. The reaction mixture was filtered and concentrated under reduced pressure using a rotary evaporator to afford 4-(2,5-dihydrofuran-3-yl)morpholine) (13.0 g, 98.7% yield) as a light yellow oil which was used directly in next step without further isolation, purification and characterization.

### Step 2: Synthesis of 1,4-dichloro-5,7-dihydrofuro[3,4-d]pyridazine

To a 200 mL sealed tube equipped with a magnetic stirring bar was added a solution of 3,6-dichloro-1,2,4,5-tetrazine (6.1 g, 40.4 mmol, 1.0 eq.) in DCM (100 mL). The resulting solution was stirred for 0.5 h at 0° C. (ice bath). Then, 4-(2,5-dihydrofuran-3-yl)morpholine (12.3 g, 79.3 mmol, 2.0 eq.) was slowly added into the reaction mixture. The resulting solution was stirred for 2 h with warming to rt. The reaction mixture was concentrated directly under reduced pressure using a rotary evaporator to obtain a brown oil. The oil was purified by reversed phase column chromatography with the following conditions: Column: XSelect CSH Prep C18 OBD Column, 19*150 mm, 5 μm; Mobile Phase A: Water (0.1% TFA), Mobile Phase B: ACN; Flow rate: 60 mL/min; Gradient: 0% B to 30% B in 30 min, 40% B; Wavelength: 220 nm; RT1: 23.1 min. After lyophilization of the combined product containing fractions, 1,4-dichloro-5,7-dihydrofuro[3,4-d]pyridazine (3.0 g, 19.8% yield) was obtained as a yellow solid. LC/MS (ESI): mass calcd for C₆H₄Cl₂N₂O: 191.01, found: m/z=192.10 [M+H]⁺. ¹H NMR (300 MHz, DMSO-d₆): δ 5.21 (s, 4H) ppm.

### Step 3: Synthesis of tert-butyl (R)-3-((4-chloro-5,7-dihydrofuro[3,4-d]pyridazin-1-yl)amino)piperidine-1-carboxylate

To a solution of 1,4-dichloro-5,7-dihydrofuro[3,4-d]pyridazine (550 mg, 2.88 mmol, 1.0 eq.) in NMP (2 mL)) were added tert-butyl (R)-3-aminopiperidine-1-carboxylate hydrochloride (577 mg, 2.88 mmol, 1 eq.), and DIEA (1.00 mL, 744 mg, 5.76 mmol, 2 eq.), and the reaction mixture was heated for 2 h under an atmosphere of N₂ at 150° C. (oil bath). After cooling to rt, the reaction mixture was quenched by addition of water (10 mL). The aqueous layer was extracted with EtOAc (3×10 mL). The combined organic phases were washed with brine (3×10 mL), dried over anhydrous Na₂SO₄, filtered, and the filtrate concentrated under reduced pressure using a rotary evaporator to afford the crude product which was further purified by silica gel column chromatography using the following conditions: Column: silica gel Column; Mobile Phase A: PE, Mobile Phase B: EA; Flow rate: 40 mL/min; Gradient: 0% B to 50% B in 30 min; Wavelength: 254 nm. After concentration of the combined product containing fractions under reduced pressure using a rotary evaporator, tert-butyl (R)-3-((4-chloro-5,7-dihydrofuro[3,4-d]pyridazin-1-yl)amino)piperidine-1-carboxylate (290 mg, 28.4% yield) was obtained. LC/MS (ESI): mass calcd for C₁₆H₂₃ClN₄O₃: 354.84, found: m/z=355.90 [M+H]⁺.

### Step 4: Synthesis of tert-butyl (R)-3-((4-(2-hydroxy-4-(trifluoromethyl)phenyl)-5,7-dihydrofuro[3,4-d]pyridazin-1-yl)amino)piperidine-1-carboxylate

To a solution of tert-butyl (R)-3-((4-chloro-5,7-dihydrofuro[3,4-d]pyridazin-1-yl)amino)piperidine-1-carboxylate (290 mg, 0.82 mmol, 1.0 eq.) and (2-hydroxy-4-(trifluoromethyl)phenyl) boronic acid (253 mg, 1.23 mmol, 1.5 eq.) in a mixture 1,4-dioxane (3 mL)) and water (0.75 mL),) were added solid NaHCO₃ (206 mg, 2.45 mmol, 3.0 eq.) and Pd(PPh₃)₄ (47.2 mg, 0.041 mmol, 0.05 eq.). After stirring for 4 h at 100° C. (oil bath) under an atmosphere of N₂, the reaction was cooled to rt and quenched with a saturated aqueous solution of NH₄Cl (5 mL). The resulting mixture was extracted with EtOAc (3×10 mL). The combined organic layers were washed with H₂O (2×5 mL), dried over anhydrous Na₂SO₄. After filtration, the filtrate was concentrated under reduced pressure using a rotary evaporator. The residue was purified by silica gel column chromatography using the following conditions: Column: silica gel Column; Mobile Phase A: PE, Mobile Phase B: EA; Flow rate: 60 mL/min; Gradient: 0% B to 50% B in 30 min; Wavelength: 254 nm. After concentration of the combined product containing fractions under reduced pressure using a rotary evaporator, tert-butyl (R)-3-((4-(2-hydroxy-4-(trifluoromethyl)phenyl)-5,7-dihydrofuro[3,4-d]pyridazin-1-yl)amino)piperidine-1-carboxylate (180 mg, 45.8% yield) was obtained. LC/MS (ESI): mass calcd, for C₂₃H₂₇F₃N₄O₄: 480.40, found: m/z=481.35 [M+H]⁺.

### Step 5: Synthesis of (R)-2-(4-(piperidin-3-ylamino)-5,7-dihydrofuro[3,4-d]pyridazin-1-yl)-5-(trifluoromethyl)phenol

To a solution of tert-butyl (R)-3-((4-(2-hydroxy-4-(trifluoromethyl)phenyl)-5,7-dihydrofuro[3,4-d]pyridazin-1-yl)amino)piperidine-1-carboxylate (180 mg, 0.38 mmol, 1.0 eq.) in 2.0 mL of 1,4-dioxane, HCl (2 mL, 1 M in 1,4-dioxane, 2 mmol, ~5 eq.) was added, and the reaction mixture was stirred for 30 min at rt. The resulting mixture was concentrated under reduced pressure using a rotary evaporator to afford (R)-2-(4-(piperidin-3-ylamino)-5,7-dihydrofuro[3,4-d]pyridazin-1-yl)-5-(trifluoromethyl)phenol hydrochloride (130 mg, 91.2% yield) which was used directly in the next step without further isolation, purification, and characterization. LC/MS (ESI): mass calcd for C₁₈H₁₉F₃N₄O₂: 380.37, found: m/z=381.20 [M+H]⁺.

### Step 6: Synthesis of tert-butyl (R)-3-(3-((4-(2-hydroxy-4-(trifluoromethyl)phenyl)-5,7-dihydrofuro[3,4-d]pyridazin-1-yl)amino)piperidin-1-yl)azetidine-1-carboxylate

To a stirred solution of (R)-2-(4-(piperidin-3-ylamino)-5,7-dihydrofuro[3,4-d]pyridazin-1-yl)-5-(trifluoromethyl)phenol hydrochloride (65 mg, 0.17 mmol, 1 eq.) in MeOH (1 mL)) was added tert-butyl 3-oxoazetidine-1-carboxylate (44 mg, 0.26 mmol, 1.5 eq.) followed by solid NaBH₃CN (32 mg, 0.51 mmol, 3.0 eq.). The reaction mixture was stirred at for 24 h at rt. The reaction mixture was quenched by addition of water (5 mL). The aqueous layer was extracted with ethyl acetate (3×15 mL), and the combined organic phases were washed with brine (3×15 mL), dried over anhydrous Na₂SO₄), filtered, and the filtrate was concentrated under reduced pressure using a rotary evaporator to afford the crude product which was further purified by silica gel column chromatography using the following conditions: Column: silica gel Column; Mobile Phase A: PE, Mobile Phase B: EtOAc; Flow rate: 40 mL/min; Gradient: 0% B to 30% B in 30 min; Wavelength: 254 nm. After concentration of the product containing fractions under reduced pressure using a rotary evaporator, tert-butyl (R)-3-(3-((4-(2-hydroxy-4-(trifluoromethyl)phenyl)-5,7-dihydrofuro[3,4-d]pyridazin-1-yl)amino)piperidin-1-yl)azetidine-1-carboxylate (30 mg, 32.8% yield) was obtained. LC/MS (ESI): mass calcd for C₂₆H₃₂F₃N₅O₄: 535.56, found: m/z=536.70 [M+H]⁺.

### Step 7: Synthesis of (R)-2-(4-((1-(azetidin-3-yl)piperidin-3-yl)amino)-5,7-dihydrofuro[3,4-d]pyridazin-1-yl)-5-(trifluoromethyl)phenol (99)

To a solution of tert-butyl (R)-3-(3-((4-(2-hydroxy-4-(trifluoromethyl)phenyl)-5,7-dihydrofuro[3,4-d]pyridazin-1-yl)amino)piperidin-1-yl)azetidine-1-carboxylate (30 mg, 0.07 mmol, 1 eq.) in 2 mL of 1,4-dioxane, HCl (2.0 mL, 1M in 1,4-dioxane, ~31 eq.) was added. The resulting mixture was stirred for 1 h at rt. Then the resulting mixture was concentrated under reduced pressure using a rotary evaporator. The residue was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min, 58% B; Wavelength: 254 nm; RT1: 5 min. After lyophilization of the combined solvent containing fractions, the target compound (R)-2-(4-((1-(azetidin-3-yl)piperidin-3-yl)amino)-5,7-dihydrofuro[3,4-d]pyridazin-1-yl)-5-(trifluoromethyl)phenol (99) (10.9 mg, 42.5% yield) was obtained as an off white solid. LC/MS (ESI): mass calcd for C₂₁H₂₄F₃N₅O₂: 435.19, found: m/z=436.20 [M+H]⁺. ¹H NMR (400 MHz, Methanol-d₄): δ 7.37 (s, 1H), 7.19-7.05 (m, 2H), 5.35-5.33 (m, 2H), 5.02-4.93 (m, 3H), 4.34-4.31 (m, 1H), 4.03-3.88 (m, 3H), 3.43-3.53 (m, 1H), 3.15-3.03 (m, 1H), 2.78-2.69 (m, 1H), 2.19-1.91 (m, 2H), 1.93-1.80 (m, 2H), 1.79-1.66 (m, 1H), 1.53-1.48 (m, 1H) ppm. ¹⁹F NMR (376 MHz, Methanol-d₄): δ −64.49 ppm.

### Example 100: (R)-2-(4-([1,4′-Bipiperidin]-3-ylamino)-5,7-dihydrofuro[3,4-d]pyridazin-1-yl)-5-(trifluoromethyl)phenol (100)

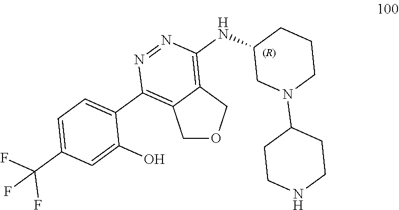

### Synthetic Route:

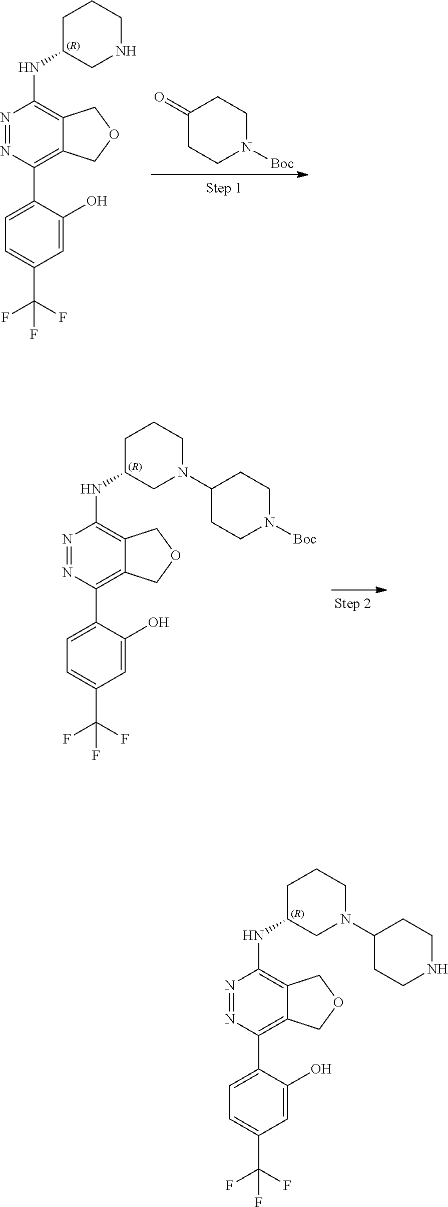

### Step 1: Synthesis of tert-butyl (R)-3-((4-(2-hydroxy-4-(trifluoromethyl)phenyl)-5,7-dihydrofuro[3,4-d]pyridazin-1-yl)amino)-[1,4′-bipiperidine]-1′-carboxylate

A solution of (R)-2-(4-(piperidin-3-ylamino)-5,7-dihydrofuro[3,4-d]pyridazin-1-yl)-5-(trifluoromethyl)phenol (300 mg, 0.79 mmol, 1 eq.) and tert-butyl 4-oxopiperidine-1-carboxylate (189 mg, 0.95 mmol, 1.2 eq.) in MeOH (6 mL). After stirring for 2 h at 60° C. (oil bath) followed by cooling to rt, solid NaBH₃CN (99 mg, 1.56 mmol, 2.0 eq.) was added and the reaction mixture was stirred for 16 h at 60° C. (oil bath). The mixture was allowed to cool down to rt, and the reaction mixture was quenched by addition of water (100 mL), the resulting mixture was extracted with EA (3×100 mL). The combined organic layers were washed with brine (200 mL), dried over anhydrous Na₂SO₄. After filtration, the filtrate was concentrated under reduced pressure using a rotary evaporator. The residue was purified by silica gel column chromatography eluting with DCM/MeOH gradients (0~10%) to afford tert-butyl (R)-3-((4-(2-hydroxy-4-(trifluoromethyl)phenyl)-5,7-dihydrofuro[3,4-d]pyridazin-1-yl)amino)-[1,4′-bipiperidine]-1′-carboxylate (200 mg, 45.0%) as a yellow oil after evaporation of the combined product containing fractions under reduced pressure using a rotary evaporator. LC/MS: MS (ESI) calcd For C₂₈H₃₆F₃N₅O₄: 563.27; found: m/z=564.30 [M+H]⁺.

### Step 2: Synthesis of (R)-2-(4-([1,4′-bipiperidin]-3-ylamino)-5,7-dihydrofuro[3,4-d]pyridazin-1-yl)-5-(trifluoromethyl)phenol (24)

To a stirred solution of tert-butyl (R)-3-((4-(2-hydroxy-4-(trifluoromethyl)phenyl)-5,7-dihydrofuro[3,4-d]pyridazin-1-yl)amino)-[1,4′-bipiperidine]-1′-carboxylate (150 mg, 0.27 mmol, 1.0 eq.) in MeOH (2 mL) was dropwise added HCl (2 mL, 4 M in 1,4-dioxane, 8 mmol, ~30 eq.) at rt. The resulting reaction mixture was stirred at rt for 3 h. The reaction was concentrated under reduced pressure using a rotary evaporator. The crude product was purified by preparative RPHPLC using the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 5% B to 95% B in 7 min, 95% B; Wavelength: 254 nm; RT1: 5.44 min; Injection Volume: 3 mL. After lyophilization of the combined product containing fractions, the target compound (R)-2-(4-([1,4′-bipiperidin]-3-ylamino)-5,7-dihydrofuro[3,4-d]pyridazin-1-yl)-5-(trifluoromethyl)phenol (100) (15.7 mg, 12.5%) was obtained as a white solid. LCMS: mass calcd for C₂₃H₂₈F₃N₅O₂: 463.51, found: m/z=464.20 [M+H]⁺. ¹H NMR (300 MHz, Methanol-d₄): δ 7.51-7.37 (m, 1H), 7.15-7.26 (m, 2H), 6.74 (d, J=7.8 Hz, 1H), 5.30-5.22 (m, 2H), 5.00-4.92 (m, 2H), 4.21-4.51 (m, 1H), 3.15-2.87 (m, 3H), 2.82-2.71 (m, 1H), 2.47-2.32 (m, 4H), 2.23-2.02 (m, 2H), 2.01-1.91 (m, 1H), 1.77-1.60 (m, 3H), 1.57-1.56 (m, 1H), 1.41-1.27 (m, 3H) ppm. ¹⁹F NMR (282 MHz, Methanol-d₄): δ −61.41 ppm.

### Example 101: (R)-1-(2-Amino-7-azaspiro[3.5]nonan-7-yl)-2-(3-((4-(2-hydroxy-4-(trifluoromethyl)phenyl)-5,7-dihydrofuro[3,4-d]pyridazin-1-yl)amino)piperidin-1-yl)ethan-1-one (101)

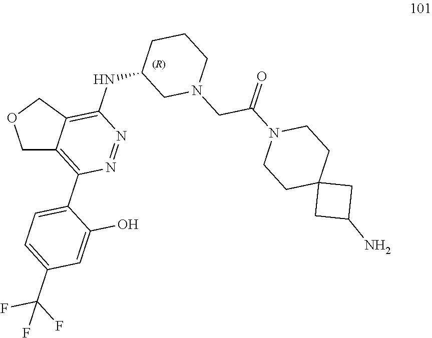

### Synthetic Route:

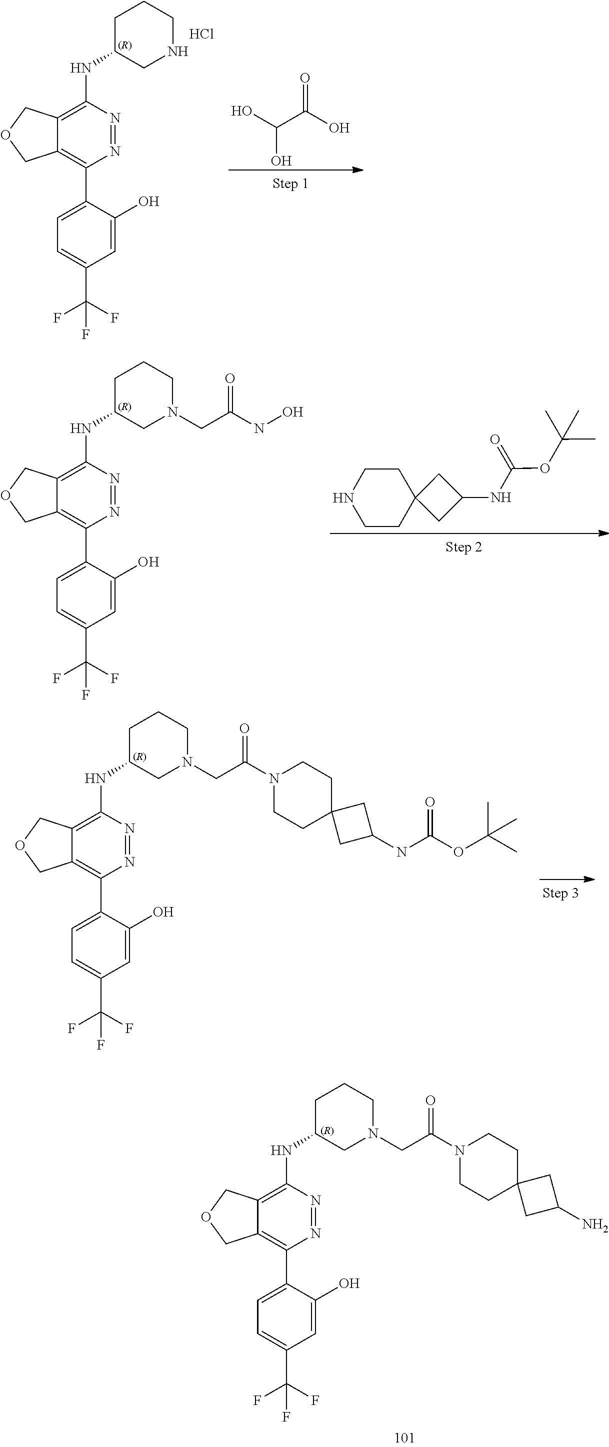

### Step 1: Synthesis of (R)-2-(3-((4-(2-hydroxy-4-(trifluoromethyl)phenyl)-5,7-dihydrofuro[3,4-d]pyridazin-1-yl)amino)piperidin-1-yl)acetic Acid

To a stirred solution of (R)-2-(4-(piperidin-3-ylamino)-5,7-dihydrofuro[3,4-d]pyridazin-1-yl)-5-(trifluoromethyl)phenol hydrochloride (650 mg, 1.70 mmol, 1.0 eq.) in MeOH (9 mL) was added 2,2-dihydroxyacetic acid (472 mg, 5.1 mmol, 3.0 eq.), and solid NaBH₃CN (322 mg, 5.1 mmol, 3.0 eq.) at rt. The resulting mixture was stirred at rt for 16 h. The reaction mixture was quenched with water/ice mixture (5 mL) and concentrated under reduced pressure using a rotary evaporator. The residue was purified by silica gel column chromatography, eluting with MeOH/DCM gradients (0~3%) to afford (R)-2-(3-((4-(2-hydroxy-4-(trifluoromethyl)phenyl)-5,7-dihydrofuro[3,4-d]pyridazin-1-yl)amino)piperidin-1-yl)acetic acid (380 mg, 50.7% yield) as a yellow solid after evaporation of the combined product containing fractions under reduced pressure using a rotary evaporator. LC/MS: mass calcd for C₂₀H₂₁F₃N₄O₄: 438.15, found: m/z=439.05 [M+H]⁺.

### Step 2: Synthesis of tert-butyl (R)-(7-(2-(3-((4-(2-hydroxy-4-(trifluoromethyl)phenyl)-5,7-dihydrofuro[3,4-d]pyridazin-1-yl)amino)piperidin-1-yl)acetyl)-7-azaspiro[3.5]nonan-2-yl)carbamate

To a stirred solution of [(3R)-3-({4-[2-hydroxy-4-(trifluoromethyl)phenyl]-5H, 7H-furo[3,4-d]pyridazin-1-yl}amino)piperidin-1-yl]acetic acid (380 mg, 0.9 mmol, 1.0 eq.) in pyridine (10 mL) was added tert-butyl (7-azaspiro[3.5]nonan-2-yl)carbamate (208 mg, 0.9 mmol, 1.0 eq.) and EDCI (249 mg, 1.3 mmol, 1.5 eq.) at rt. The resulting mixture was stirred for 16 h at 50° C. The reaction mixture was concentrated under reduced pressure using a rotary evaporator. The residue was purified by silica gel column chromatography, eluted with MeOH/DCM gradients (0~3%) to afford tert-butyl (R)-(7-(2-(3-((4-(2-hydroxy-4-(trifluoromethyl)phenyl)-5,7-dihydrofuro[3,4-d]pyridazin-1-yl)amino)piperidin-1-yl)acetyl)-7-azaspiro[3.5]nonan-2-yl)carbamate (300 mg, 52.4% yield) as a yellow solid after evaporation of the combined product containing fractions using a rotary evaporator. LC/MS: mass calcd for C₃₃H₄₃F₃N₆O₅: 660.32, found: m/z=661.25 [M+H]⁺.

### Step 3: Synthesis of (R)-1-(2-amino-7-azaspiro[3.5]nonan-7-yl)-2-(3-((4-(2-hydroxy-4-(trifluoromethyl)phenyl)-5,7-dihydrofuro[3,4-d]pyridazin-1-yl)amino)piperidin-1-yl)ethan-1-one (101)

Into a 50 mL round-bottomed flask were added tert-butyl (R)-(7-(2-(3-((4-(2-hydroxy-4-(trifluoromethyl)phenyl)-5,7-dihydrofuro[3,4-d]pyridazin-1-yl)amino)piperidin-1-yl)acetyl)-7-azaspiro[3.5]nonan-2-yl)carbamate (300 mg, 0.5 mmol, 1.0 eq.), HCl (2 mL, 4 M in 1,4-dioxane, 8 mmol, 16 eq.) and EtOAc (4 mL). The reaction mixture was stirred for 3 h at rt. The reaction mixture was concentrated under reduced pressure using a rotary evaporator. The residue was purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 25% B to 55% B in 7 min; Wavelengths: 254 nm/220 nm; RT1: 6.08 min). This resulted in the target compound (R)-1-(2-amino-7-azaspiro[3.5]nonan-7-yl)-2-(3-((4-(2-hydroxy-4-(trifluoromethyl)phenyl)-5,7-dihydrofuro[3,4-d]pyridazin-1-yl)amino)piperidin-1-yl) ethan-1-one (101) (47.3 mg, 18.6% yield) as a light yellow solid after lyophilization of the combined product containing fractions. LC/MS: mass calcd for C₂₈H₃₅F₃N₆O₃: 560.27, found: m/z=561.30 [M+H]⁺. ¹H NMR (400 MHz, Methanol-d₄): δ 7.42 (d, J=8.8 Hz, 1H), 7.25-7.15 (m, 2H), 5.45-5.35 (m, 2H), 5.12-5.02 (m, 2H), 4.42-4.30 (m, 1H), 3.80-3.40 (m, 5H), 3.33-3.28 (m, 1H), 3.20-3.15 (m, 1H), 3.10-3.00 (m, 1H), 2.75-2.58 (m, 1H), 2.40-2.10 (m, 4H), 2.00-1.80 (m, 2H), 1.78-1.46 (m, 8H) ppm. ¹⁹F NMR (376 MHz, Methanol-d₄): δ −64.52 ppm.

### Example 102: (R)-2-(3-((6-(2-Hydroxy-4-(trifluoromethyl)phenyl)-5-methylpyridazin-3-yl)amino)piperidin-1-yl)-1-(2,7-diazaspiro[3.5]nonan-7-yl)ethan-1-one (102)

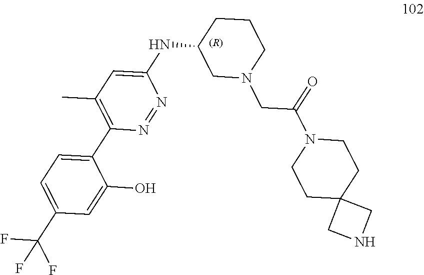

### Synthetic Route:

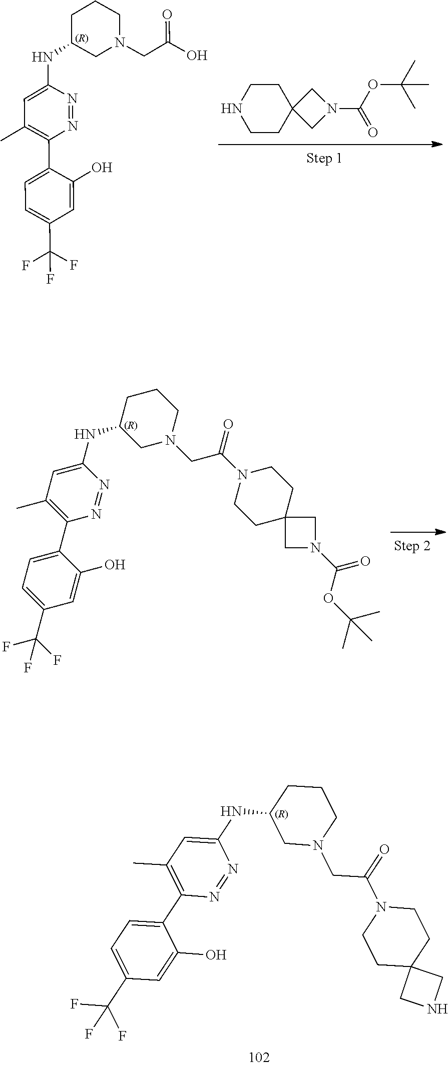

### Step 1: Synthesis of tert-butyl (R)-7-(2-(3-((6-(2-hydroxy-4-(trifluoromethyl)phenyl)-5-methylpyridazin-3-yl)amino)piperidin-1-yl)acetyl)-2,7-diazaspiro[3.5]nonane-2-carboxylate

To a stirred solution of (R)-2-(3-((6-(2-hydroxy-4-(trifluoromethyl)phenyl)-5-methylpyridazin-3-yl)amino)piperidin-1-yl)acetic acid (210 mg, 0.5 mmol, 1.0 eq.) in pyridine (5 mL) was added tert-butyl 2,7-diazaspiro[3.5]nonane-2-carboxylate (116 mg, 0.5 mmol, 1.0 eq.) and EDCI (147 mg, 0.8 mmol, 1.5 eq.) in portions at rt. The resulting mixture was stirred at 50° C. for 16 h. The resulting mixture was concentrated under reduced pressure using a rotary evaporator. The residue was purified by silica gel column chromatography, eluting with MeOH/DCM gradients (0~10%) to afford tert-butyl (R)-7-(2-(3-((6-(2-hydroxy-4-(trifluoromethyl)phenyl)-5-methylpyridazin-3-yl)amino)piperidin-1-yl)acetyl)-2,7-diazaspiro[3.5]nonane-2-carboxylate (200 mg, 63.3% yield) as a yellow solid after evaporation of the combined product containing fractions under reduced pressure using a rotary evaporator. LC/MS (ESI): mass calcd for C₃₁H₄₁F₃N₆O₄: 618.31, found: m/z=619.35 [M+H]⁺.

### Step 2: Synthesis of (R)-2-(3-((6-(2-hydroxy-4-(trifluoromethyl)phenyl)-5-methylpyridazin-3-yl)amino)piperidin-1-yl)-1-(2,7-diazaspiro[3.5]nonan-7-yl)ethan-1-one (102)

A solution of tert-butyl (R)-7-(2-(3-((6-(2-hydroxy-4-(trifluoromethyl)phenyl)-5-methylpyridazin-3-yl)amino)piperidin-1-yl)acetyl)-2,7-diazaspiro[3.5]nonane-2-carboxylate (200 mg, 0.3 mmol, 1.0 eq.) in THF (2 mL) was treated with HCl (1 mL, 4 M in 1,4-dioxane, 4 mmol, ~13 eq.) at rt for 2.0 h. The resulting mixture was concentrated under reduced pressure using a rotary evaporator to afford—a crude product (200.0 mg). The crude product was purified by preparative RP-HPLC using the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 17% B to 47% B in 7 min; Wavelengths: 254 nm/220 nm; RT1: 5.57 min) to afford the target compound (R)-2-(3-((6-(2-hydroxy-4-(trifluoromethyl)phenyl)-5-methylpyridazin-3-yl)amino)piperidin-1-yl)-1-(2,7-diazaspiro[3.5]nonan-7-yl)ethan-1-one (102) (35.9 mg, 21.2%) as a white solid after lyophilization of the combined product containing fractions. LC/MS (ESI): mass calcd for C₂₆H₃₃F₃N₆O₂: 518.26, found: m/z=519.30 [M+H]⁺. ¹H NMR (400 MHz, Methanol-d₄): δ 7.40 (d, J=8.0 Hz, 1H), 7.19 (d, J=8.0 Hz, 1H), 7.16 (s, 1H), 6.78 (s, 1H), 4.20-4.10 (m, 1H), 3.70-3.50 (m, 8H), 3.30-3.20 (m, 2H), 3.00-2.85 (m, 1H), 2.70-2.50 (m, 1H), 2.55-2.25 (m, 2H), 2.15 (s, 3H), 2.08-1.65 (m, 7H), 1.60-1.40 (m, 1H) ppm. ¹⁹F NMR (376 MHz, *Methanol-d₄): δ −64.28 ppm.

### Example 103: (R)-2-(3-((6-(2-Hydroxy-4-(trifluoromethyl)phenyl)-5-methylpyridazin-3-yl)amino)piperidin-1-yl)-1-(piperazin-1-yl)ethan-1-one (103)

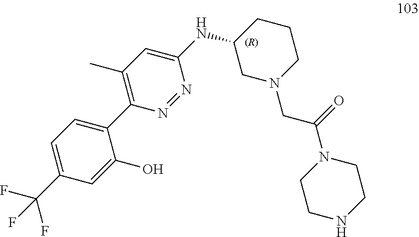

### Synthetic Route:

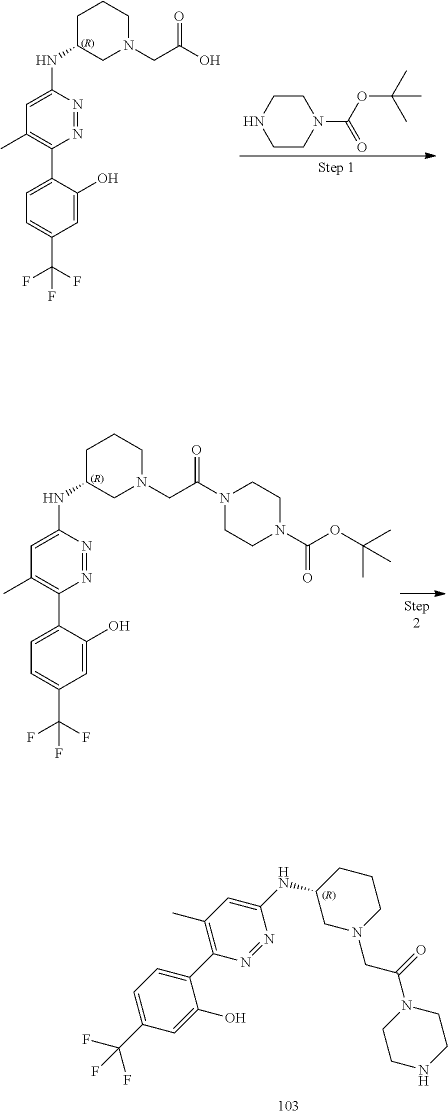

### Step 1: Synthesis of tert-butyl (R)-4-(2-(3-((6-(2-hydroxy-4-(trifluoromethyl)phenyl)-5-methylpyridazin-3-yl)amino)piperidin-1-yl)acetyl) piperazine-1-carboxylate

To a stirred solution of (R)-2-(3-((6-(2-hydroxy-4-(trifluoromethyl)phenyl)-5-methylpyridazin-3-yl)amino)piperidin-1-yl)acetic acid (220 mg, 0.5 mmol, 1.0 eq.) in pyridine (3 mL) was added tert-butyl piperazine-1-carboxylate (100 mg, 0.5 mmol, 1.0 eq.) and EDCI (154 mg, 0.8 mmol, 1.5 eq.) in portions at rt. The resulting mixture was stirred at 50° C. for 16 h. The reaction mixture was poured into water/ice (10 mL) and was extracted with EA (3×10 mL). The organic phases were combined and washed with H₂O (3×20 mL) and dried over anhydrous Na₂SO₄. After filtration, the filtrate was concentrated under reduced pressure using a rotary evaporator. The residue was purified by silica gel column chromatography, eluting with MeOH/DCM gradients (0~3%) to afford tert-butyl (R)-4-(2-(3-((6-(2-hydroxy-4-(trifluoromethyl)phenyl)-5-methylpyridazin-3-yl)amino)piperidin-1-yl)acetyl) piperazine-1-carboxylate (240 mg, 77.4% yield) as a yellow oil after evaporation of the combined product containing fractions under reduced pressure using a rotary evaporator. LC/MS (ESI): mass calcd for C₂₈H₃₇F₃N₆O₄: 578.28, found: m/z=579.25 [M+H]⁺.

### Step 2: Synthesis of (R)-2-(3-((6-(2-yydroxy-4-(trifluoromethyl)phenyl)-5-methylpyridazin-3-yl)amino)piperidin-1-yl)-1-(piperazin-1-yl)ethan-1-one (103)

A solution of tert-butyl (R)-4-(2-(3-((6-(2-hydroxy-4-(trifluoromethyl)phenyl)-5-methylpyridazin-3-yl)amino)piperidin-1-yl)acetyl) piperazine-1-carboxylate (200 mg, 0.3 mmol, 1.0 eq.) in HCl (2 mL, 4 M in 1,4-dioxane, 8 mmol, ~27 eq.) and THF (2 mL) was stirred for 3 h at rt. The resulting mixture was concentrated under vacuum using a rotary evaporator. The crude product was washed with Et₂O (5 mL) to get a crude product (150 mg). The crude product was purified by preparative RP-HPLC using the following conditions: Column: XBridge Shield RP 18 OBD Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 23% B to 53% B in 7 min; Wavelength: 254 nm; RT1: 6.35 min, to afford the target compound (R)-2-(3-((6-(2-yydroxy-4-(trifluoromethyl)phenyl)-5-methylpyridazin-3-yl)amino)piperidin-1-yl)-1-(piperazin-1-yl)ethan-1-one (103) (65.4 mg, 38.5% yield) as a white solid after lyophilization of the combined product containing fractions. LC/MS (ESI): mass calcd for C₂₃H₂₉F₃N₆O₂: 478.23, found: m/z=479.25 [M+H]⁺. ¹H NMR (400 MHz, Methanol-d₄): δ: 7.41 (d, J=8.0 Hz, 1H), 7.24 (d, J=8.0 Hz, 1H), 7.18 (s, 1H), 6.79 (s, 1H), 4.20-4.10 (s, 1H), 3.80-3.52 (m, 5H), 3.25-3.15 (m, 2H), 3.00-2.80 (m, 4H), 2.70-2.50 (m, 1H), 2.40-2.20 (m, 2H), 2.15 (s, 3H), 2.00-1.80 (m, 2H), 1.75-1.50 (m, 1H), 1.70-1.45 (m, 1H) ppm. ¹⁹F NMR (376 MHz, Methanol-d₄): δ −64.29 ppm.

### Example 104: (R)-2-(4-Methyl-6-((1-(2-(piperazin-1-yl)ethyl)piperidin-3-yl)amino)pyridazin-3-yl)-5-(trifluoromethyl)phenol (104)

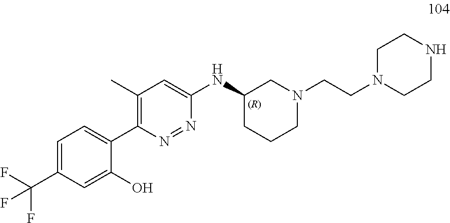

### Synthetic Route:

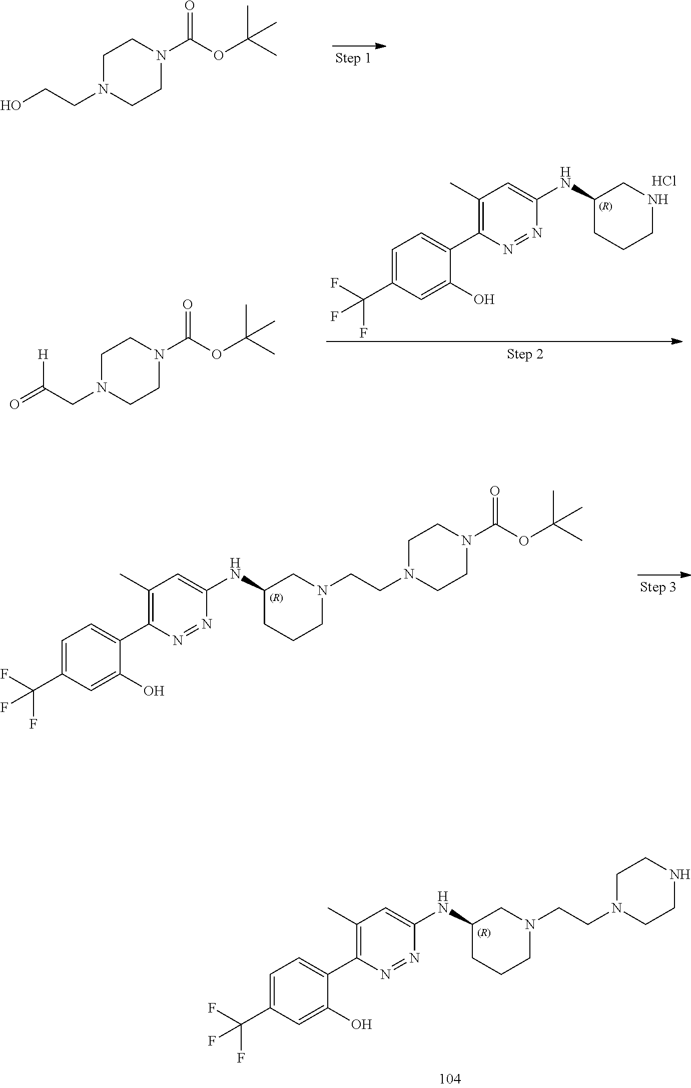

### Step 1: Synthesis of tert-butyl 4-(2-oxoethyl) piperazine-1-carboxylate

To a stirred solution of DMSO (0.7 mL, 10.5 mmol, 15.0 eq.) in DCM (4 mL) was added oxalyl chloride (0.3 mL, 450 mg, 3.5 mmol, 5.0 eq.) slowly at −78° C. (dry ice/acetone bath) for 0.5 h under an atmosphere of N₂. Then, tert-butyl 4-(2-hydroxyethyl) piperazine-1-carboxylate (150 mg, 0.7 mmol, 1.0 eq.) and TEA (1.4 mL, 1.02 g, 10.5 mmol, 15.0 eq.) were added at −78° C. The resulting solution was stirred with warming to rt for 2 h. The reaction mixture was quenched by addition of a water/ice mixture (10 mL). The mixture was neutralized to pH=7 with a saturated aqueous NaHCO₃ solution. The resulting mixture was extracted with DCM (3×20 mL) and the combined organic layers were dried over anhydrous Na₂SO₄. After filtration, the filtrate was concentrated under reduced pressure using a rotary evaporator to afford tert-butyl 4-(2-oxoethyl) piperazine-1-carboxylate (230 mg, crude) as a yellow solid. The crude product was used directly in the next step without further isolation, purification, and characterization. LC/MS: MS (ESI) calcd for C₁₁H₂₀N₂O₃: 228.15. Found: m/z=229.15 [M+H]⁺.

### Step 2: Synthesis of tert-butyl (R)-4-(2-(3-((6-(2-hydroxy-4-(trifluoromethyl)phenyl)-5-methylpyridazin-3-yl)amino)piperidin-1-yl)ethyl) piperazine-1-carboxylate

To a stirred mixture of tert-butyl 4-(2-oxoethyl) piperazine-1-carboxylate (230 mg, 1.0 mmol, 2.0 eq.) and (R)-2-(4-methyl-6-(piperidin-3-ylamino)pyridazin-3-yl)-5-(trifluoromethyl)phenol hydrochloride (177 mg, 0.5 mmol, 1.0 eq.) in MeOH (5 mL) was added NaBH₃CN (126 mg, 2.0 mmol, 4.0 eq.) in small portions at 0° C. (ice bath). The resulting mixture was stirred with warming to rt for 16 h followed by concentration under reduced pressure using a rotary evaporator. The residue was purified by silica gel column chromatography eluting with EtOAc/PE gradients (0~10%) to afford tert-butyl (R)-4-(2-(3-((6-(2-hydroxy-4-(trifluoromethyl)phenyl)-5-methylpyridazin-3-yl)amino)piperidin-1-yl)ethyl) piperazine-1-carboxylate (100 mg, 17.6% yield) as a yellow solid after evaporation of the combined product containing fractions under reduced pressure using a rotary evaporator. LC/MS: MS (ESI) calcd for C₂₈H₃₉F₃N₆O₃: 564.30; found: m/z=565.40 [M+H]⁺.

### Step 3: Synthesis of (R)-2-(4-methyl-6-((1-(2-(piperazin-1-yl)ethyl)piperidin-3-yl)amino)pyridazin-3-yl)-5-(trifluoromethyl)phenol (104)

To a stirred mixture of tert-butyl (R)-4-(2-(3-((6-(2-hydroxy-4-(trifluoromethyl)phenyl)-5-methylpyridazin-3-yl)amino)piperidin-1-yl)ethyl) piperazine-1-carboxylate (100 mg, 0.2 mmol, 1.0 eq.) in THF (2 mL) was added HCl (1 mL, 4 M 1,4-in dioxane, 4 mmol, 20 eq.) at 0° C. (ice bath). The resulting mixture was stirred with warming to rt for overnight followed by concentration under reduced pressure using a rotary evaporator The crude product (100 mg) was further purified by preparative-HPLC using the following conditions: Column: XBridge Shield RP 18 OBD Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 23% B to 53% B in 7 min; Wavelength: 254 nm; RT1: 6.32 min to afford the target compound (R)-2-(4-methyl-6-((1-(2-(piperazin-1-yl)ethyl)piperidin-3-yl)amino)pyridazin-3-yl)-5-(trifluoromethyl)phenol (104) (14.4 mg, 17.5% yield) as a white solid after lyophilization of the combined product containing fractions. LC/MS: MS (ESI) calcd for C₂₃H₃₁F₃N₆O: 464.25; found: m/z=465.25 [M+H]⁺. ¹H NMR (300 MHz, DMSO-d₆): δ 7.38 (d, J=8.1 Hz, 1H), 7.21 (d, J=8.4 Hz, 1H), 7.15 (s, 1H), 6.77 (s, 1H), 4.20-4.05 (m, 1H), 3.20-3.05 (s, 1H), 3.00-2.80 (m, 4H), 2.70-2.55 (m, 1H), 2.60-2.45 (m, 8H), 2.35-2.20 (m, 1H), 2.18-2.05 (m, 4H), 2.00-1.88 (m, 1H), 1.90-1.80 (m, 1H), 1.70-1.60 (m, 1H), 1.45-1.35 (m, 1H) ppm. ¹⁹F NMR (282 MHz, DMSO-d₆): δ −64.29 ppm.

### Example 105: (R)-2-(6-((1-(Azetidin-3-ylmethyl)piperidin-3-yl)amino)-4-methylpyridazin-3-yl)-5-(trifluoromethyl)phenol (105)

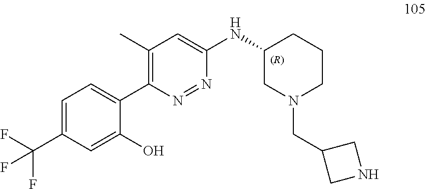

### Synthetic Route:

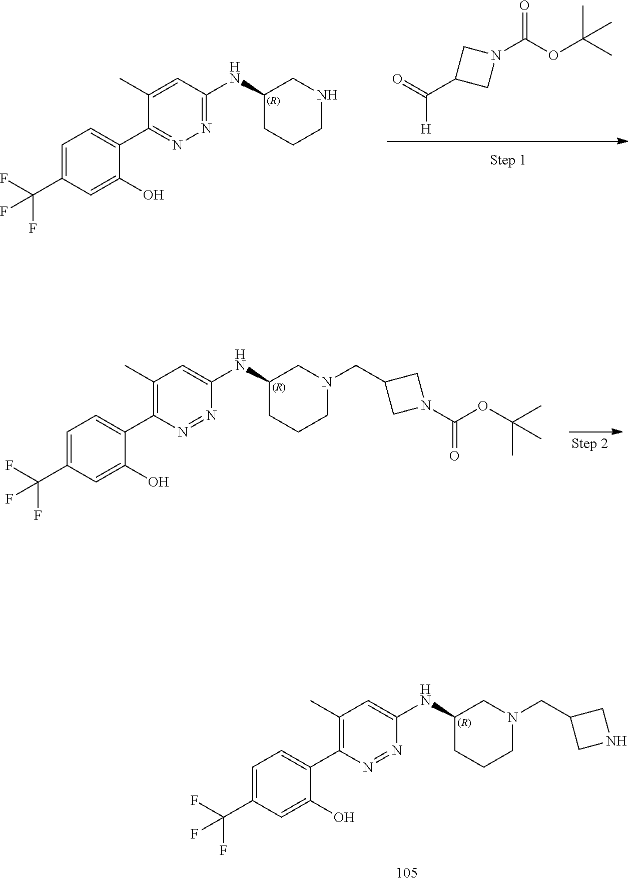

### Step 1: Synthesis of tert-butyl (R)-3-((3-((6-(2-hydroxy-4-(trifluoromethyl)phenyl)-5-methylpyridazin-3-yl)amino)piperidin-1-yl)methyl)azetidine-1-carboxylate

To a stirred solution of (R)-2-(4-methyl-6-(piperidin-3-ylamino)pyridazin-3-yl)-5-(trifluoromethyl)phenol hydrochloride (350 mg, 1.0 mmol, 1.0 eq.) in MeOH (9 mL) were added tert-butyl 3-formylazetidine-1-carboxylate (552 mg, 3.0 mmol, 3.0 eq.) and NaBH₃CN (187.3 mg, 3.0 mmol, 3.0 eq.) in small portions at rt. The resulting mixture was stirred at rt for 16 h. The reaction was quenched with ice/water (5 mL) and the volatiles were removed under reduced pressure using a rotary evaporator. The residue was purified by silica gel column chromatography eluting with DCM/MeOH gradients (0~3%) to afford tert-butyl (R)-3-((3-((6-(2-hydroxy-4-(trifluoromethyl)phenyl)-5-methylpyridazin-3-yl)amino)piperidin-1-yl)methyl)azetidine-1-carboxylate (500 mg, 96.5%) as a yellow solid after evaporation of the combined product containing fractions under reduced pressure using a rotary evaporator. LC/MS: MS (ESI) calcd for C₂₆H₃₄F₃N₅O₃: 521.26, found: m/z=522.40 [M+H]⁺.

### Step 2: Synthesis of (R)-2-(6-((1-(azetidin-3-ylmethyl)piperidin-3-yl)amino)-4-methylpyridazin-3-yl)-5-(trifluoromethyl)phenol (105)

Into a 50 mL flask were added tert-butyl (R)-3-((3-((6-(2-hydroxy-4-(trifluoromethyl)phenyl)-5-methylpyridazin-3-yl)amino)piperidin-1-yl)methyl)azetidine-1-carboxylate (100 mg, 1.0 mmol, 1.0 eq.) and DCM (5 mL) followed by addition of TFA (1.3 mL). The reaction mixture was stirred for 3 h at rt. The reaction mixture was concentrated under reduced pressure using a rotary evaporator, and the residue was purified by reversed-phase flash chromatography using the following conditions: Column, C18 silica gel; mobile phase, ACN in Water (10 mmol/L NH₄HCO₃+0.1% NH₃), 10% to 40% gradient in 10 min; detector wavelength UV 254 nm to afford the target compound (R)-2-(6-((1-(azetidin-3-ylmethyl)piperidin-3-yl)amino)-4-methylpyridazin-3-yl)-5-(trifluoromethyl)phenol (105) (24.9 mg, 6.1% yield) as a white solid after lyophilization of the combined product containing fractions. LC/MS: MS (ESI) calcd for C₂₁H₂₆F₃N₅O: 421.20, found: m/z=422.20 [M+H]⁺. ¹H NMR (400 MHz, Methanol-d₄): δ 7.37 (d, J=7.8 Hz, 1H), 7.30-7.13 (m, 2H), 6.77 (s, 1H), 4.15-4.05 (m, 1H), 3.85-3.75 (m, 2H), 3.62-3.50 (m, 2H), 3.15-2.98 (m, 2H), 2.80-2.44 (m, 3H), 2.30-2.05 (m, 5H), 2.00-1.90 (m, 1H), 1.85-1.76 (m, 1H), 1.71-1.60 (m, 1H), 1.50-1.35 (m, 1H) ppm. ¹⁹F NMR (376 MHz, Methanol-d₄): δ −64.26 ppm.

### Example 106:2-(4-Methyl-6-(((R)-1-(((S)-pyrrolidin-2-yl)methyl)piperidin-3-yl)amino)pyridazin-3-yl)-5-(trifluoromethyl)phenol (106)

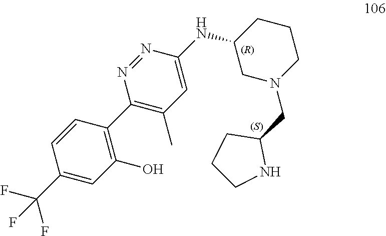

### Synthetic Route:

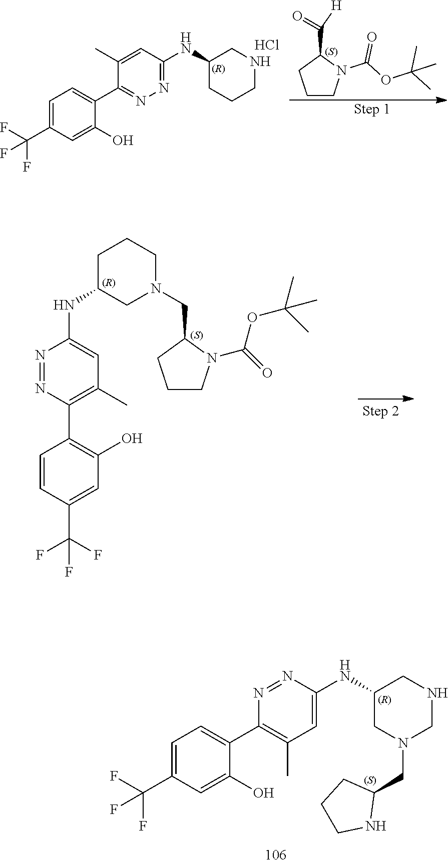

### Step 1: Synthesis of tert-butyl(S)-2-(((R)-3-((6-(2-hydroxy-4-(trifluoromethyl)phenyl)-5-methylpyridazin-3-yl)amino)piperidin-1-yl)methyl)pyrrolidine-1-carboxylate

To a stirred solution of (R)-2-(4-methyl-6-(piperidin-3-ylamino)pyridazin-3-yl)-5-(trifluoromethyl)phenol hydrochloride (200 mg, 0.6 mmol, 1.0 eq.) in MeOH (3 mL) was added tert-butyl(S)-2-formylpyrrolidine-1-carboxylate (339 mg, 1.7 mmol, 3.0 eq.) and NaBH₃CN (45.4 mg, 1.2 mmol, 2.0 eq.) in small portions at 0° C. (ice bath). The reaction mixture was stirred with warming to rt for 16 h and poured into water/ice (10 mL) to quench the reaction. The reaction mixture was extracted by EA (3×10 mL), and the organic phases were combined and dried over anhydrous Na₂SO₄. After filtration, the filtrate was concentrated under reduced pressure using a rotary evaporator. The residue was purified by silica gel column chromatography eluting with MeOH/DCM gradients (0~5%) to afford tert-butyl(S)-2-(((R)-3-((6-(2-hydroxy-4-(trifluoromethyl)phenyl)-5-methylpyridazin-3-yl)amino)piperidin-1-yl)methyl)pyrrolidine-1-carboxylate (250 mg, 74.0% yield) as a brown oil after evaporation of the combined product containing fractions under reduced pressure using a rotary evaporator. LC/MS (ESI): mass calcd for C₂₇H₃₆F₃N₅O₃: 535.28, found: m/z=536.30 [M+H]⁺.

### Step 2: Synthesis of 2-(4-methyl-6-(((R)-1-(((S)-pyrrolidin-2-yl)methyl)piperidin-3-yl)amino)pyridazin-3-yl)-5-(trifluoromethyl)phenol (106)

To a stirred solution of tert-butyl(S)-2-(((R)-3-((6-(2-hydroxy-4-(trifluoromethyl)phenyl)-5-methylpyridazin-3-yl)amino)piperidin-1-yl)methyl)pyrrolidine-1-carboxylate (200 mg, 0.4 mmol, 1.0 eq.) in DCM (4 mL) was dropwise added TFA (2 mL) at rt. The resulting mixture was stirred at rt for 4 h followed by concentration under reduced pressure using a rotary evaporator. The residue was dissolved in DMF (1 mL) and purified by preparative RP-HPLC using the following conditions: Column: XBridge Prep OBD C18 Column, 19*250 mm, 5 μm; Mobile Phase A: Water (10 mmol/L NH₄HCO₃), Mobile Phase B: ACN; Flow rate: 25 mL/min; Gradient: 29% B to 59% B in 7 min; Wavelengths: 254 nm/220 nm; RT1: 5 min) to afford the target compound 2-(4-methyl-6-(((R)-1-(((S)-pyrrolidin-2-yl)methyl)piperidin-3-yl)amino)pyridazin-3-yl)-5-(trifluoromethyl)phenol (106) (31.6 mg, 18.8% yield) as a white solid after lyophilization of the combined product containing fractions. LC/MS (ESI): mass calcd for C₂₂H₂₈F₃N₅O: 435.22, found: m/z=436.15 [M+H]⁺. ¹H NMR (400 MHz, Methanol-d₄): δ 7.37 (d, J=8.0 Hz, 1H), 7.24-7.10 (m, 2H), 6.79 (s, 1H), 4.18-4.08 (m, 1H), 3.65-3.55 (m, 1H), 3.25-3.15 (m, 1H), 3.12-3.00 (m, 2H), 2.90-2.80 (m, 1H), 2.65-2.55 (m, 2H), 2.30-2.15 (m, 5H), 2.10-1.80 (m, 5H), 1.78-1.60 (m, 1H), 1.70-1.50 (m, 2H) ppm. ¹⁹F NMR (376 MHz, Methanol-d₄): δ −64.25 ppm.

### Example 107:2-(4-Methyl-6-(((R)-1-(((R)-pyrrolidin-2-yl)methyl)piperidin-3-yl)amino)pyridazin-3-yl)-5-(trifluoromethyl)phenol (107)

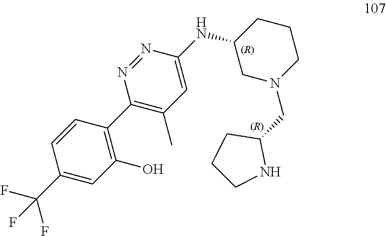

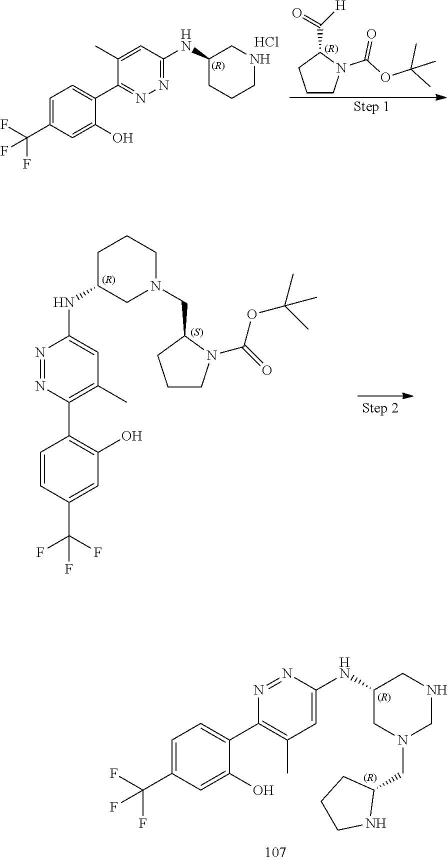

### Step 1: Synthesis of tert-butyl (R)-2-(((R)-3-((6-(2-hydroxy-4-(trifluoromethyl)phenyl)-5-methylpyridazin-3-yl)amino)piperidin-1-yl)methyl)pyrrolidine-1-carboxylate

To a stirred solution of (R)-2-(4-methyl-6-(piperidin-3-ylamino)pyridazin-3-yl)-5-(trifluoromethyl)phenol hydrochloride (200 mg, 0.6 mmol, 1.0 eq.) in MeOH (3 mL) was added tert-butyl (R)-2-formylpyrrolidine-1-carboxylate (339 mg, 1.7 mmol, 3.0 eq.) and NaBH₃CN (45.4 mg, 1.2 mmol, 2.0 eq.) in small portions at 0° C. (ice bath). The reaction mixture was stirred with warming to rt for 16 h and poured into water (10 mL) to quench the reaction. The reaction mixture was extracted with EA (3×10 mL), washed with brine (1×10 mL) and dried over anhydrous Na₂SO₄. After filtration, the filtrate was concentrated under reduced pressure using a rotary evaporator. The residue was purified by silica gel column chromatography eluting with MeOH/DCM gradients (0~5%) to afford tert-butyl (R)-2-(((R)-3-((6-(2-hydroxy-4-(trifluoromethyl)phenyl)-5-methylpyridazin-3-yl)amino)piperidin-1-yl)methyl)pyrrolidine-1-carboxylate (250 mg, 74.0% yield) as a brown oil after evaporation of the combined product containing fractions under reduced pressure using a rotary evaporator. LC/MS (ESI): mass calcd for C₂₇H₃₆F₃N₅O₃: 535.28, found: m/z=536.25 [M+H]⁺.

### Step 2: Synthesis of 2-(4-methyl-6-(((R)-1-(((R)-pyrrolidin-2-yl)methyl)piperidin-3-yl)amino)pyridazin-3-yl)-5-(trifluoromethyl)phenol (107)

To a stirred solution of tert-butyl (R)-2-(((R)-3-((6-(2-hydroxy-4-(trifluoromethyl)phenyl)-5-methylpyridazin-3-yl)amino)piperidin-1-yl)methyl)pyrrolidine-1-carboxylate (250 mg, 0.5 mmol, 1.0 eq.) in DCM (2 mL) was dropwise added TFA (0.5 mL) at rt and the resulting mixture was stirred at rt for 4 h. The reaction mixture was concentrated under reduced pressure using a rotary evaporator. The residue was dissolved in DMF (1.0 mL) and was further purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 25% B to 39% B in 10 min; Wavelengths: 254 nm/220 nm; RT1: 6.97 min) to afford the target compound 2-(4-methyl-6-(((R)-1-(((R)-pyrrolidin-2-yl)methyl)piperidin-3-yl)amino)pyridazin-3-yl)-5-(trifluoromethyl)phenol (107) (13.4 mg, 6.3% yield) as a white solid after lyophilization of the combined product containing fractions. LC/MS (ESI): mass calcd for C₂₂H₂₈F₃N₅O: 435.22, found: m/z=436.15 [M+H]⁺. ¹H NMR (400 MHz, Methanol-d₄): δ 7.41 (d, J=8.0 Hz, 1H), 7.25-7.23 (m, 1H), 7.18 (s, 1H), 6.80 (s, 1H), 4.08-3.98 (m, 1H), 3.88-3.78 (m, 1H), 3.50-3.40 (m, 3H), 2.80-2.70 (m, 1H), 2.50-2.40 (m, 2H), 2.35-2.25 (m, 1H), 2.10-1.98 (m, 7H), 1.85-1.75 (m, 3H), 1.70-1.65 (m, 1H), 1.50-1.35 (m, 1H) ppm. ¹⁹F NMR (376 MHz, Methanol-d₄): δ −64.30 ppm.

### Example 108: (R)-2-(4-Methyl-6-((1-(2-((oxetan-3-ylmethyl)amino)ethyl)piperidin-3-yl)amino)pyridazin-3-yl)-5-(trifluoromethyl)phenol (108)

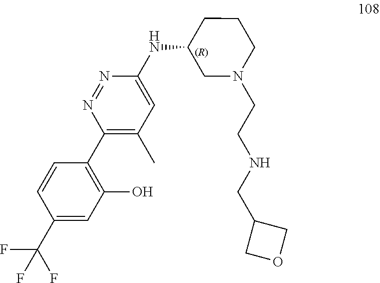

### Synthetic Route:

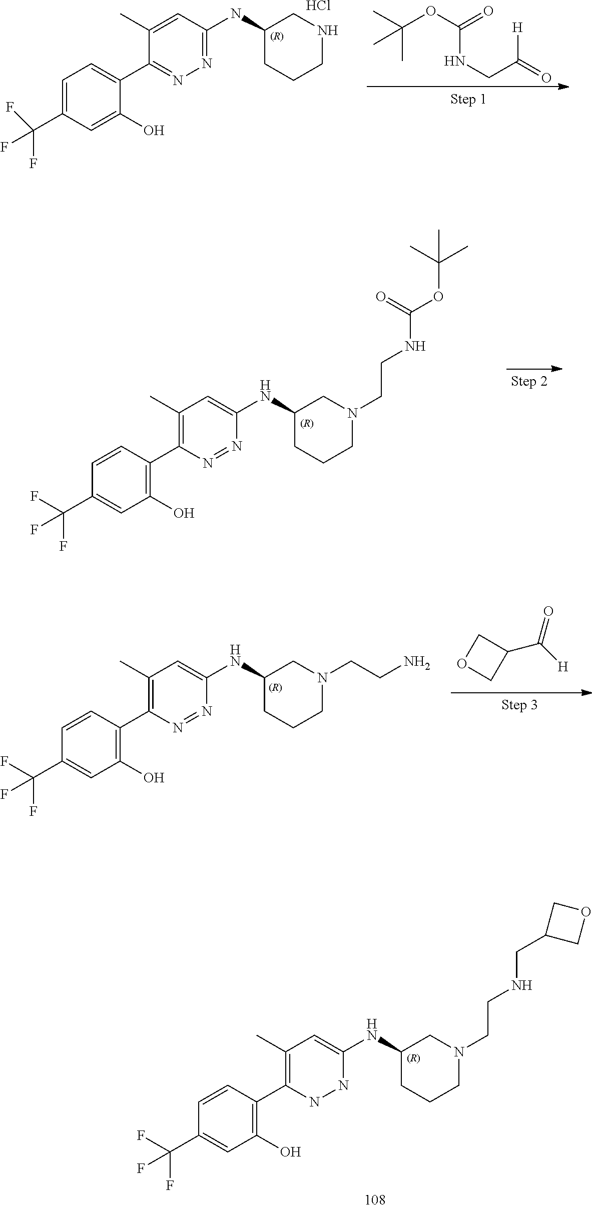

### Step 1: Synthesis of tert-butyl (R)-(2-(3-((6-(2-hydroxy-4-(trifluoromethyl)phenyl)-5-methylpyridazin-3-yl)amino)piperidin-1-yl)ethyl)carbamate

To a stirred mixture of (R)-2-(4-methyl-6-(piperidin-3-ylamino)pyridazin-3-yl)-5-(trifluoromethyl)pheno hydrochloride (1.0 g, 2.8 mmol, 1.0 eq.) and tert-butyl(2-oxoethyl)carbamate (0.7 g, 4.3 mmol, 1.5 eq.) in MeOH (12 mL) was added NaBH₃CN (360 mg, 5.7 mmol, 2.0 eq.) in small portions at 0° C. (ice bath). The resulting mixture was stirred with warming to rt for 16 h. The resulting mixture was concentrated under reduced pressure using a rotary evaporator. The residue was purified by silica gel column chromatography eluting with MeOH/DCM gradients (0~9%) to afford tert-butyl (R)-(2-(3-((6-(2-hydroxy-4-(trifluoromethyl)phenyl)-5-methylpyridazin-3-yl)amino)piperidin-1-yl)ethyl)carbamate (500 mg, 35.6%) as a yellow solid after evaporation of the combined product containing fractions under reduced pressure using a rotary evaporator. LC/MS (ESI): mass calcd for C₂₄H₃₂F₃N₅O₃: 495.25, found: m/z=496.30 [M+H]⁺.

### Step 2: Synthesis of (R)-2-(6-((1-(2-aminoethyl)piperidin-3-yl)amino)-4-methylpyridazin-3-yl)-5-(trifluoromethyl)phenol hydrochloride

To a stirred mixture of tert-butyl (R)-(2-(3-((6-(2-hydroxy-4-(trifluoromethyl)phenyl)-5-methylpyridazin-3-yl)amino)piperidin-1-yl)ethyl)carbamate (450 mg, 0.9 mmol, 1.0 eq.) in THF (6 mL) was added HCl (3 mL, 4 M in 1,4-dioxane, 12 mmol, ~13 eq.) at 0° C. (ice bath). The resulting mixture was stirred with warming to rt for overnight followed by concentration under reduced pressure using a rotary evaporator to afford crude (R)-2-(6-((1-(2-aminoethyl)piperidin-3-yl)amino)-4-methylpyridazin-3-yl)-5-(trifluoromethyl)phenol hydrochloride (400 mg) as a yellow solid. The crude product was used directly in the next step without further isolation, purification, and characterization. LC/MS (ESI): mass calcd for C₁₉H₂₄F₃N₅O: 395.19, found: m/z=320.90 [M+H]⁺.

### Step 3: Synthesis of (R)-2-(4-methyl-6-((1-(2-((oxetan-3-ylmethyl)amino)ethyl)piperidin-3-yl)amino)pyridazin-3-yl)-5-(trifluoromethyl)phenol (108)

To a stirred mixture of (R)-2-(6-((1-(2-aminoethyl)piperidin-3-yl)amino)-4-methylpyridazin-3-yl)-5-(trifluoromethyl)phenol hydrochloride (240 mg, 0.6 mmol, 1.0 eq.) and oxetane-3-carbaldehyde (48 mg, 0.6 mmol, 0.9 eq.) in MeOH (4 mL) was added NaBH₃CN (115 mg, 1.8 mmol, 3.0 eq.) in small portions at 0° C. (ice bath). The resulting mixture was stirred with warming to rt for 16 h. The reaction was quenched with water/ice (2 mL), and the resulting mixture was concentrated under reduced pressure using a rotary evaporator. The residue was purified by silica gel column chromatography eluting with MeOH/DCM gradients (0~17%) to afford pre-purified (R)-2-(4-methyl-6-((1-(2-((oxetan-3-ylmethyl)amino)ethyl)piperidin-3-yl)amino)pyridazin-3-yl)-5-(trifluoromethyl)phenol (108) (80 mg, pre-purified) after evaporation of the combined product containing fractions under reduced pressure using a rotary evaporator. The pre-purified product (80 mg) was further purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 21% B to 35% B in 10 min; Wavelengths: 254 nm/220 nm; RT1: 10 min) to afford the target compound (R)-2-(4-methyl-6-((1-(2-((oxetan-3-ylmethyl)amino)ethyl)piperidin-3-yl)amino)pyridazin-3-yl)-5-(trifluoromethyl)phenol (108) (15.6 mg, 5.3%) as a white solid after lyophilization of the combined product containing fractions. LC/MS (ESI): mass calcd for C₂₃H₃₀F₃N₅O₂: 465.24, found: m/z=466.20 [M+H]⁺. ¹H NMR (300 MHz, Methanol-d₄): δ 7.38 (d, J=7.8 Hz, 1H), 7.20 (d, J=8.1 Hz, 1H), 7.15 (s, 1H), 6.78 (s, 1H), 4.85-4.70 (m, 2H), 4.50-4.35 (m, 2H), 4.20-4.05 (m, 1H), 3.25-3.10 (m, 1H), 3.05-2.90 (m, 3H), 2.80-2.70 (m, 2H), 2.68-2.60 (m, 1H), 2.58-2.48 (m, 2H), 2.35-2.22 (m, 1H), 2.20-2.15 (m, 1H), 2.13 (s, 3H), 2.00-1.90 (m, 1H), 1.88-1.78 (m, 1H), 1.75-1.60 (m, 1H), 1.55-1.38 (m, 1H) ppm. ¹⁹F NMR (282 MHz, Methanol-d₄): δ −64.29 ppm.

### Example 109: (R)-4-(((2-(3-((6-(2-Hydroxy-4-(trifluoromethyl)phenyl)-5-methylpyridazin-3-yl)amino)piperidin-1-yl)ethyl)amino)methyl)tetrahydro-2H-thiopyran 1,1-dioxide (109)

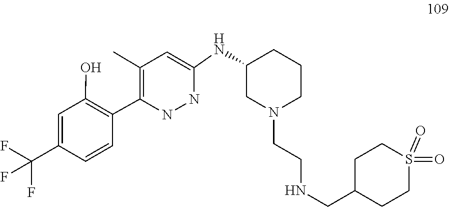 [SMILES: CC1=C(c2ccc(C(F)(F)F)cc2O)NNC(N[C@@H]2CCCN(CCNCC3CCS(=O)(=O)CC3)C2)=C1]

### Synthetic Route:

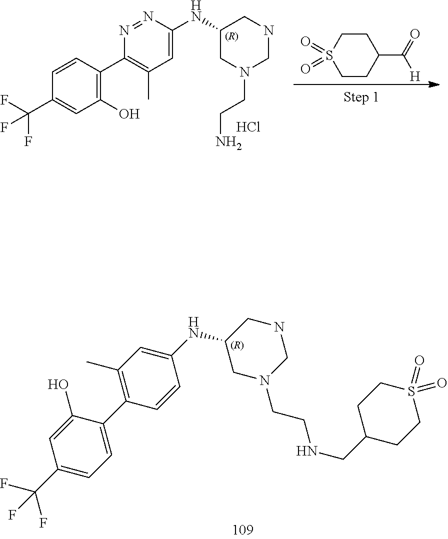

### Step 1: Synthesis of (R)-4-(((2-(3-((6-(2-hydroxy-4-(trifluoromethyl)phenyl)-5-methylpyridazin-3-yl)amino)piperidin-1-yl)ethyl)amino)methyl)tetrahydro-2H-thiopyran 1,1-dioxide (109)

To a stirred mixture of (R)-2-(6-((1-(2-aminoethyl)piperidin-3-yl)amino)-4-methylpyridazin-3-yl)-5-(trifluoromethyl)phenol hydrochloride (150 mg, 0.4 mmol, 1.0 eq.) and tetrahydro-2H-thiopyran-4-carbaldehyde 1,1-dioxide (62 mg, 0.4 mmol, 1.0 eq.) in MeOH (5 mL) was added NaBH₃CN (72.0 mg, 1.1 mmol, 3.0 eq) in small portions at 0° C. (ice bath). The resulting mixture was stirred with warming to rt for 1 h. The resulting reaction mixture was concentrated under reduced pressure using a rotary evaporator. The crude product was dissolved in DMF (1 mL) and purified by preparative RP-HPLC using the following conditions: Column: YMC-Actus Triart C18 ExRS19*250 mm, 5 μm; Mobile Phase A: Water (10 mmol/L NH₄HCO₃+0.1% NH₃), Mobile Phase B: ACN; Flow rate: 25 mL/min mL/min; Gradient: 35% B to 65% B in 7 min; Wavelength: 254 nm; RT1: 6.55 min to afford (R)-4-(((2-(3-((6-(2-hydroxy-4-(trifluoromethyl)phenyl)-5-methylpyridazin-3-yl)amino)piperidin-1-yl)ethyl)amino)methyl)tetrahydro-2H-thiopyran 1,1-dioxide (109) (16.0 mg, 7.6% yield) as a white solid after lyophilization of the combined product containing fractions. LC/MS (ESI): mass calcd for C₂₅H₃₄F₃N₅O₃S: 541.23, found: m/z=542.15 [M+H]⁺. ¹H NMR (300 MHz, Methanol-d₄): δ 7.39 (d, J=7.8 Hz, 1H), 7.21 (d, J=8.1 Hz, 1H), 7.16 (s, 1H), 6.77 (s, 1H), 4.18-4.05 (m, 1H), 3.20-2.97 (m, 5H), 2.85-2.65 (m, 3H), 2.60-2.40 (m, 4H), 2.38-2.25 (m, 1H), 2.20-2.10 (m, 6H), 2.00-1.90 (m, 1H), 2.88-2.60 (m, 5H), 2.55-2.35 (m, 1H) ppm. ¹⁹F NMR (282 MHz, Methanol-d₄): δ −64.29 ppm.

### Example 110:2-((R)-3-((6-(2-Hydroxy-4-(trifluoromethyl)phenyl)-5-methylpyridazin-3-yl)amino)piperidin-1-yl)-1-((R)-3-methylpiperazin-1-yl)ethan-1-one (110)

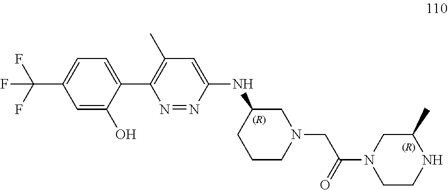

### Synthetic Route:

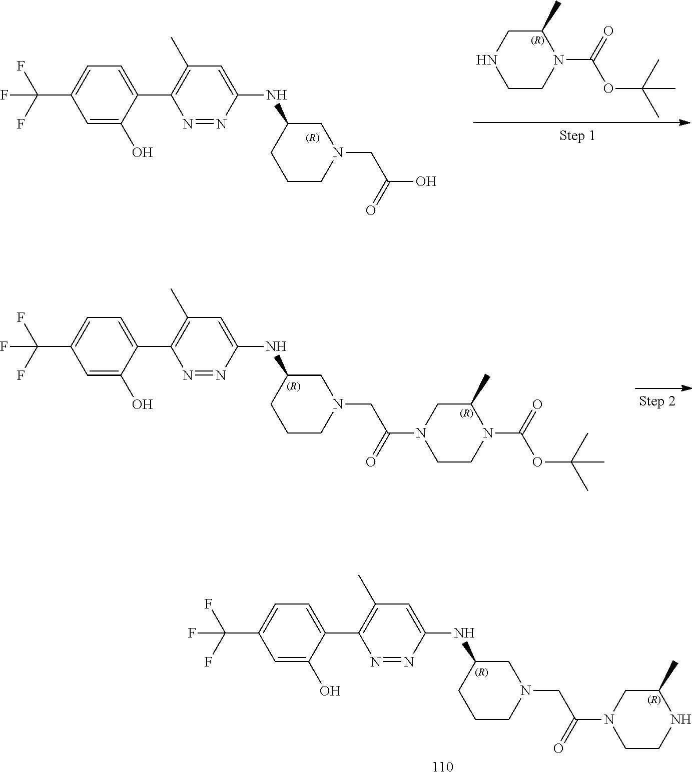

### Step 1: Synthesis of tert-butyl (R)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate

To a stirred solution of [(3R)-3-({6-[2-hydroxy-4-(trifluoromethyl)phenyl]-5-methylpyridazin-3-yl}amino)piperidin-1-yl]acetic acid (200 mg, 0.49 mmol, 1.0 eq.) in DMF (5 mL) were added EDCI (140 mg, 0.7 mmol, 1.5 eq.), HOBt (65.9 mg, 0.5 mmol, 1.0 eq.), DIEA (255 μL, 189 mg, 1.5 mmol, 3.0 eq.) and tert-butyl (R)-2-methylpiperazine-1-carboxylate (107 mg, 0.5 mmol, 1.1 eq.) in portions at 0° C. (ice bath). The resulting mixture was stirred at rt for 16 h. The reaction mixture was poured into water/ice (10 mL) to quench the reaction. The reaction mixture was extracted with EA (3×10 mL). The organic phases were combined and washed with H₂O (3×20 mL) and dried over anhydrous Na₂SO₄. After filtration, the filtrate was concentrated under reduced pressure using a rotary evaporator. The residue was purified by silica gel column chromatography eluting with MeOH/DCM gradients (0~3%) to afford tert-butyl (R)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate (220 mg, 69.5% yield) as a yellow oil after evaporation of the combined product containing fractions under reduced pressure using a rotary evaporator. LC/MS (ESI): mass calcd for C₂₉H₃₉F₃N₆O₄: 592.30, found: m/z=593.30 [M+H]⁺.

### Step 2: Synthesis of 2-((R)-3-((6-(2-hydroxy-4-(trifluoromethyl)phenyl)-5-methylpyridazin-3-yl)amino)piperidin-1-yl)-1-((R)-3-methylpiperazin-1-yl)ethan-1-one (110)

To a stirred solution of tert-butyl (R)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate (200 mg, 0.3 mmol, 1.0 eq.) in DCM (2 mL) was dropwise added TFA (0.5 mL) at 0° C. (ice bath). The resulting mixture was stirred at rt for 1 h. The reaction mixture was concentrated using a rotary evaporator and the residue was dissolved in DMF (1.0 mL), then purified by Prep-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 21% B to 35% B in 10 min; Wavelength: 254 nm/220 nm; RT1: 10 min) to afford the target compound 2-((R)-3-((6-(2-hydroxy-4-(trifluoromethyl)phenyl)-5-methylpyridazin-3-yl)amino)piperidin-1-yl)-1-((R)-3-methylpiperazin-1-yl)ethan-1-one (110) (53.0 mg, 31.6% yield) as a white solid after lyophilization of the combined product containing fractions. LC/MS (ESI): mass calcd for C₂₄H₃₁F₃N₆O₂: 492.25, found: m/z=493.25 [M+H]⁺. ¹H NMR (400 MHz, Methanol-d₄): δ 7.41 (d, J=8.0 Hz, 1H), 7.23 (d, J=8.0 Hz, 1H), 7.18 (s, 1H), 6.79 (d, J=8.0 Hz, 1H), 4.40-4.30 (m, 1H), 4.20-4.08 (m, 2H), 3.52-3.40 (m, 1H), 3.20-3.10 (m, 2H), 3.08-2.89 (m, 3H), 2.80-2.70 (m, 1H), 2.68-2.52 (m, 2H), 2.45-2.25 (m, 2H), 2.15 (s, 3H), 2.00-1.80 (m, 2H), 1.75-1.40 (m, 2H), 1.20-1.05 (m, 3H) ppm. ¹⁹F NMR (376 MHz, Methanol-d₄): δ −64.29 ppm.

### Example 111:2-((R)-3-((6-(2-Hydroxy-4-(trifluoromethyl)phenyl)-5-methylpyridazin-3-yl)amino)piperidin-1-yl)-1-((S)-3-methylpiperazin-1-yl)ethan-1-one (111)

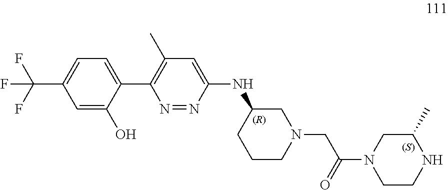

### Synthetic Route:

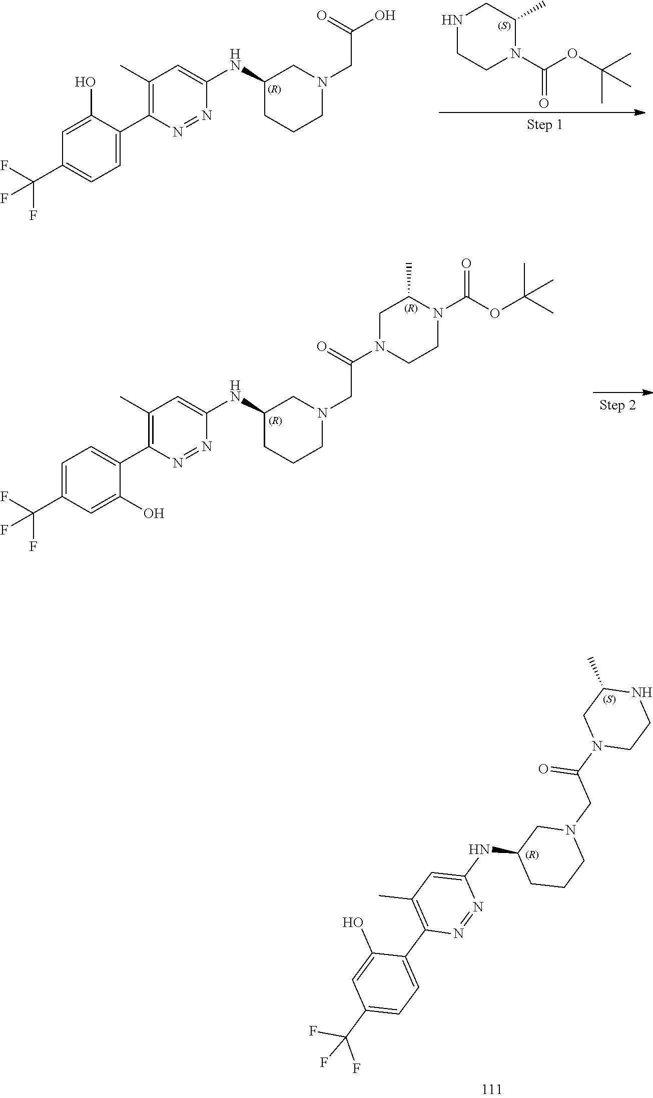

### Step 1: Synthesis of tert-butyl(S)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate

To a stirred solution of [(3R)-3-({6-[2-hydroxy-4-(trifluoromethyl)phenyl]-5-methylpyridazin-3-yl}amino)piperidin-1-yl]acetic acid (200 mg, 0.48 mmol, 1.0 eq.) and tert-butyl(S)-2-methylpiperazine-1-carboxylate (102 mg, 0.5 mmol, 1.1 eq.) in DMF (5 mL) were added EDCI (134 mg, 0.7 mmol, 1.5 eq.), HOBt (63 mg, 0.5 mmol, 1.0 eq.) and DIEA (243 μL, 180 mg, 1.2 mmol, 3.0 eq.) in portions at 0° C. (ice bath). The resulting mixture was stirred with warming to rt for 16 h. The reaction mixture was poured into a water/ice mixture (10 mL) for quenching of the reaction. The reaction mixture was extracted with EA (3×10 mL). The organic phases were combined and washed with H₂O (3×20 mL) and dried over anhydrous Na₂SO₄. After filtration, the filtrate was concentrated under reduced pressure using a rotary evaporator. The residue was purified by silica gel column chromatography, eluting with MeOH/DCM gradients (0~3%) to afford tert-butyl(S)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate (230 mg, 78.7% yield) as a yellow oil after evaporation of the combined product containing fractions under reduced pressure using a rotary evaporator. LC/MS (ESI): mass calcd for C₂₉H₃₉F₃N₆O₄: 592.30, found: m/z=593.30 [M+H]⁺.

### Step 2: Synthesis of 2-((R)-3-((6-(2-hydroxy-4-(trifluoromethyl)phenyl)-5-methylpyridazin-3-yl)amino)piperidin-1-yl)-1-((S)-3-methylpiperazin-1-yl)ethan-1-one (111)

To a stirred solution of tert-butyl(S)-4-(2-((R)-3-((6-(2-hydroxy-4-(trifluoromethyl)phenyl)-5-methylpyridazin-3-yl)amino)piperidin-1-yl)acetyl)-2-methylpiperazine-1-carboxylate (200 mg, 0.3 mmol, 1.0 eq.) in DCM (1 mL) was dropwise added TFA (0.5 mL) at rt, and the resulting mixture was stirred at rt for 1 h. The reaction mixture was concentrated under reduced pressure using a rotary evaporator and the residue was dissolved in DMF (1 mL) and purified by preparative RP-HPLC using the following conditions: Column: XBridge Prep OBD C18 Column, 30*150 mm, 5 μm; Mobile Phase A: Water (10 mmol/L NH₄HCO₃+0.1% NH₃), Mobile Phase B: ACN; Flow rate: 60 mL/min mL/min; Gradient: 18% B to 38% B in 10 min; Wavelength: 254 nm/220 nm; RT1: 9.95 min) to afford the target compound 2-((R)-3-((6-(2-hydroxy-4-(trifluoromethyl)phenyl)-5-methylpyridazin-3-yl)amino)piperidin-1-yl)-1-((S)-3-methylpiperazin-1-yl)ethan-1-one (111) (46.7 mg, 27.7% yield) as a white solid after lyophilization of the combined product containing fractions. LC/MS (ESI): mass calcd for C₂₄H₃₁F₃N₆O₂: 492.25, found: m/z=493.25 [M+H]⁺. ¹H NMR (400 MHz, Methanol-d₄): δ: 7.41 (d, J=8.0 Hz, 1H), 7.23 (d, J=8.0 Hz, 1H), 7.18 (s, 1H), 6.80 (d, J=8.0 Hz, 1H), 4.40-4.30 (m, 1H), 4.20-4.08 (m, 2H), 3.52-3.40 (m, 1H), 3.20-3.05 (m, 2H), 3.02-2.88 (m, 3H), 2.80-2.70 (m, 2H), 2.68-2.55 (m, 1H), 2.45-2.25 (m, 2H), 2.15 (s, 3H), 2.00-1.80 (m, 2H), 1.75-1.60 (m, 1H), 1.58-1.40 (m, 1H), 1.20-1.05 (m, 3H) ppm. ¹⁹F NMR (376 MHz, Methanol-d₄): δ −64.29 ppm.

### Example 112:1-((3aR,6aS)-Hexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (112)

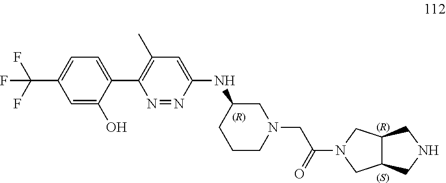

### Synthetic Route:

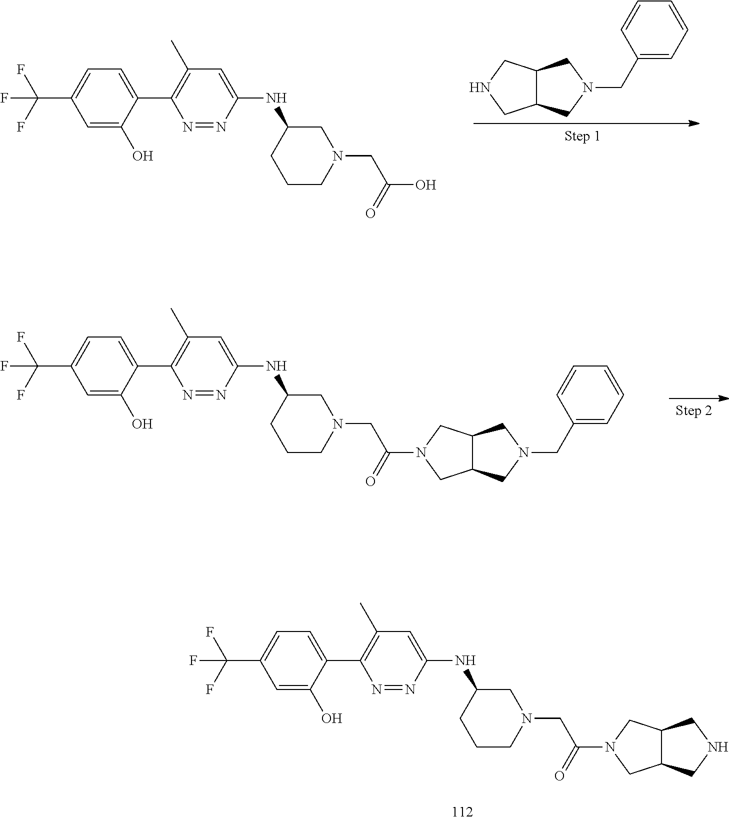 [SMILES: Cc1cc(N[C@@H]2CCCN(CC(=O)O)C2)nnc1-c1ccc(C(F)(F)F)cc1O ; c1ccc(CN2C[C@H]3CNC[C@H]3C2)cc1 ; Cc1cc(N[C@@H]2CCCN(CC(=O)N3C[C@H]4CN(Cc5ccccc5)C[C@H]4C3)C2)nnc1-c1ccc(C(F)(F)F)cc1O ; Cc1cc(N[C@@H]2CCCN(CC(=O)N3C[C@H]4CNC[C@H]4C3)C2)nnc1-c1ccc(C(F)(F)F)cc1O]

### Step 1: Synthesis of 1-((3aR,6aS)-5-benzylhexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one

A solution of (R)-2-(3-((6-(2-hydroxy-4-(trifluoromethyl)phenyl)-5-methylpyridazin-3-yl)amino)piperidin-1-yl)acetic acid (200 mg, 0.5 mmol, 1.0 eq.) and (3aR,6aS)-2-benzyloctahydropyrrolo[3,4-c]pyrrole (148 mg, 0.7 mmol, 1.5 eq.) in DMF (4 mL) was treated with DIEA (250 μL, 186 mg, 1.5 mmol, 3.0 eq.), and HOBt (66 mg, 0.5 mmol, 1.0 eq.) at rt under an atmosphere of N₂ followed by addition of EDCI (140 mg, 0.7 mmol, 1.5 eq.) in portions at 0° C. (ice bath). The reaction mixture was stirred overnight at rt. The resulting mixture was poured into ice-water (10 mL) and extracted with EA (3×20 mL). The combined organic layers were washed with H₂O (3×30 mL), dried over anhydrous Na₂SO₄. After filtration, the filtrate was concentrated under reduced pressure using a rotary evaporator. The residue was purified by silica gel column chromatography eluting with MeOH/DCM gradients (0~9%) to afford 1-((3aR,6aS)-5-benzylhexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (200 mg, 69.0% yield) as a yellow solid after evaporation of the combined product containing fractions under reduced pressure using a rotary evaporator. LC/MS (ESI): mass calcd for C₃₂H₃₇F₃N₆O₂: 594.29, found: m/z=595.35 [M+H]⁺.

### Step 2: Synthesis of 1-((3aR,6aS)-hexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (112)

To a solution of 1-((3aR,6aS)-5-benzylhexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (150 mg, 0.3 mmol, 1.0 eq.) in DMF (6 mL) was added 10 wt-% Pd/C (150 mg, 100% w/w.). The reaction mixture was stirred for 16 h at 80° C. under an atmosphere of H₂. The resulting mixture was filtered over Celite®, and the filter cake was washed with DMF (3×5 mL). The resulting mixture was concentrated under reduced pressure using a rotary evaporator. The residue was purified by silica gel column chromatography eluting with MeOH/DCM gradients (0~9%) to afford the pre-purified target compound 1-((3aR,6aS)-hexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (112) (50 mg, pre-purified) after evaporation of the combined product containing fractions under reduced pressure using a rotary evaporator. The product product was further purified by preparative RP-HPLC using the following conditions: Column: XBridge Prep OBD C18 Column, 30*150 mm, 5 μm; Mobile Phase A: Water (10 mmol/L NH₄HCO₃+0.1 vol-% NH₃ aq.), Mobile Phase B: ACN; Flow rate: 60 mL/min; Gradient: 16% B to 46% B in 7 min; Wavelengths: 254 nm/220 nm; RT1: 6 min to afford the target compound 1-((3aR,6aS)-hexahydropyrrolo[3,4-c]pyrrol-2 (1H)-yl)-2-((R)-3-((6-(2-hydroxy-4-(trifluoromethyl)phenyl)-5-methylpyridazin-3-yl)amino)piperidin-1-yl)ethan-1-one (112) (7.1 mg, 5.6% yield) as a white solid after lyophilization of the combined product containing fractions. LC/MS (ESI): mass calcd for C₂₅H₃₁F₃N₆O₂: 504.25, found: m/z=505.20 [M+H]⁺. ¹H NMR (400 MHz, Methanol-d₄): δ 7.42-7.39 (m, 1H), 7.30-7.20 (m, 1H), 7.17 (s, 1H), 6.85-6.75 (m, 1H), 4.20-4.00 (m, 1H), 3.90-3.72 (m, 1H), 3.70-3.60 (m, 2H), 3.55-3.48 (m, 1H), 3.45-3.40 (m, 1H), 3.30-3.20 (m, 2H), 3.19-3.12 (m, 1H), 3.11-2.85 (m, 4H), 2.78-2.68 (m, 2H), 2.60-2.20 (m, 2H), 2.15 (s, 3H), 2.00-1.80 (m, 2H), 1.78-1.40 (m, 2H) ppm. ¹⁹F NMR (376 MHz, Methanol-d₄): δ −64.29 ppm.

### Example 113: (R)-2-(3-((6-(4-hydroxybenzo[b]thiophen-5-yl)-5-methylpyridazin-3-yl)amino)piperidin-1-yl)-1-(piperazin-1-yl)ethan-1-one (113)

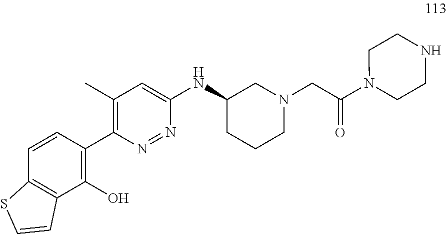 [SMILES: Cc1cc(N[C@@H]2CCCN(CC(=O)N3CCNCC3)C2)nnc1-c1ccc2sccc2c1O]

### Synthetic Scheme:

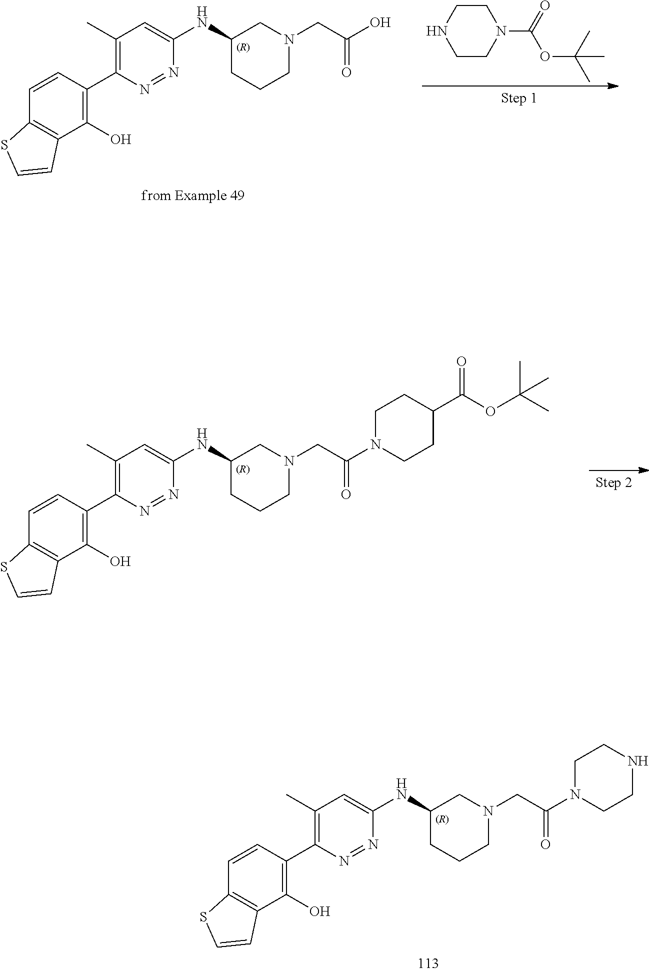

### Step 1: Synthesis of tert-butyl (R)-4-(2-(3-((6-(4-hydroxybenzo[b]thiophen-5-yl)-5-methylpyridazin-3-yl)amino)piperidin-1-yl)acetyl) piperazine-1-carboxylate

In a single-necked flask, [(3R)-3-{[6-(4-hydroxy-1-benzothiophen-5-yl)-5-methylpyridazin-3-yl]amino}piperidin-1-yl]acetic acid (prepared as disclosed for Example 49) (160 mg, 0.4 mmol, 1.0 eq.) was dissolved in 4 mL of pyridine followed by addition of EDCI (154 mg, 0.8 mmol, 2.0 eq.) and tert-butyl piperazine-1-carboxylate (112 mg, 0.6 mmol, 1.5 eq.) under an atmosphere of nitrogen. The reaction mixture was stirred at 50° C. overnight (heating mantle). Upon completion, the reaction mixture was allowed to cool to room temperature, diluted with 30 mL of water and extracted with 3×20 mL EtOAc. The combined organic extracts were washed with 50 mL of brine and dried over anhydrous sodium sulfate. After filtration, the filtrate was concentrated by rotary evaporation under reduced pressure. The residue was purified by flash column chromatography (8% MeOH in DCM) to afford tert-butyl (R)-4-(2-(3-((6-(4-hydroxybenzo[b]thiophen-5-yl)-5-methylpyridazin-3-yl)amino)piperidin-1-yl)acetyl) piperazine-1-carboxylate (130 mg, 57%) as a yellow oil after evaporation of the combined product containing fractions under reduced pressure using a rotary evaporator. LC/MS: mass calcd, for C₂₉H₃₈N₆O₄S 566.27, found m/z=511.20 [M-Bu]⁺. ¹H NMR (400 MHz, Methanol-d₄): δ 7.56 (d, J=5.6 Hz, 1H), 7.46 (d, J=7.0 Hz, 2H), 7.24 (d, J=8.3 Hz, 1H), 6.80 (s, 1H), 4.16-4.05 (m, 1H), 3.67-3.59 (m, 2H), 3.56-3.49 (m, 3H), 3.47-3.40 (m, 2H), 3.39-3.30 (m, 1H), 3.26-3.21 (m, 2H), 2.98-2.88 (m, 1H), 2.64-2.54 (m, 1H), 2.41-2.22 (m, 2H), 2.19 (s, 3H), 1.95-1.78 (m, 2H), 1.73-1.58 (m, 1H), 1.51-1.45 (m, 1H), 1.44 (s, 9H) ppm.

### Step 2: Synthesis of (R)-2-(3-((6-(4-Hydroxybenzo[b]thiophen-5-yl)-5-methylpyridazin-3-yl)amino)piperidin-1-yl)-1-(piperazin-1-yl)ethan-1-one (113)

In a single-necked flask, tert-butyl (R)-4-(2-(3-((6-(4-hydroxybenzo[b]thiophen-5-yl)-5-methylpyridazin-3-yl)amino)piperidin-1-yl)acetyl) piperazine-1-carboxylate (130 mg, 0.2 mmol, 1.0 eq.) was dissolved in HCl/1,4-dioxane (5 mL, 2 M, 10 mmol, 50 eq.). The reaction mixture was stirred at rt for 1 h. Upon completion, the reaction mixture was concentrated by rotary evaporation under reduced pressure. The residue was purified by preparative RP HPLC (5-65% ACN in H₂O, 0.1% HCOOH) to afford the target compound (R)-2-(3-((6-(4-Hydroxybenzo[b]thiophen-5-yl)-5-methylpyridazin-3-yl)amino)piperidin-1-yl)-1-(piperazin-1-yl)ethan-1-one (113) (62 mg, 57%) as a yellow solid after lyophilization of the combined product containing fractions. LC/MS: mass calcd for C₂₄H₃₀N₆O₂S, 466.22, found m/z=467.15, [M+H]⁺. ¹H NMR (400 MHz, Methanol-d₄): δ 7.58 (d, J=5.5 Hz, 1H), 7.48 (d, J=6.9 Hz, 2H), 7.24 (d, J=8.1 Hz, 1H), 6.81 (s, 1H), 4.18-4.08 (m, 1H), 3.83-3.72 (m, 1H), 3.69-3.57 (m, 2H), 3.52-3.43 (m, 1H), 3.28-3.17 (m, 2H), 3.03-2.92 (m, 2H), 2.91-2.76 (m, 3H), 2.69-2.58 (m, 1H), 2.37-2.25 (m, 1H), 2.20 (s, 3H), 1.96-1.89 (m, 1H), 1.88-1.79 (m, 1H), 1.73-1.61 (m, 1H), 1.55-1.43 (m, 1H) ppm.

### Example 114: (R)-5-(6-((1-(azetidin-3-yl)piperidin-3-yl)amino)-4-methylpyridazin-3-yl)benzo[b]thiophen-4-ol (114)

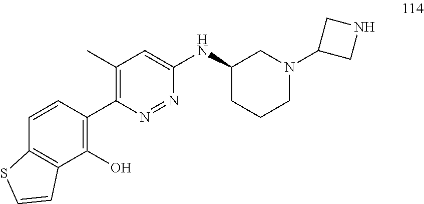 [SMILES: Cc1cc(N[C@@H]2CCCN(C3CNC3)C2)nnc1-c1ccc2sccc2c1O]

### Synthetic Route:

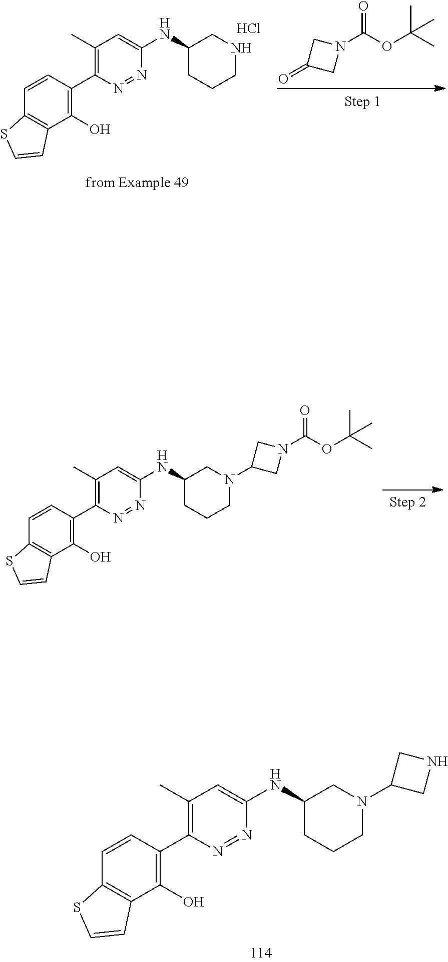 [SMILES: Cc1cc(N[C@@H]2CCCNC2)nnc1-c1ccc2sccc2c1O ; Cl ; CC(C)(C)OC(=O)N1CC(=O)C1 ; Cc1cc(N[C@@H]2CCCN(C3CN(C(=O)OC(C)(C)C)C3)C2)nnc1-c1ccc2sccc2c1O ; Cc1cc(N[C@@H]2CCCN(C3CNC3)C2)nnc1-c1ccc2sccc2c1O]

### Step 1: Synthesis of tert-butyl (R)-3-(3-((6-(4-hydroxybenzo[b]thiophen-5-yl)-5-methylpyridazin-3-yl)amino)piperidin-1-yl)azetidine-1-carboxylate

In a single-necked flask, (R)-5-(4-methyl-6-(piperidin-3-ylamino)pyridazin-3-yl)benzo[b]thiophen-4-ol hydrochloride (prepared as disclosed in Example 49) (150 mg, 0.4 mmol, 1.0 eq.) and tert-butyl 3-oxoazetidine-1-carboxylate (188 mg, 1.0 mmol, 2.5 eq.) were dissolved in 3 mL of MeOH followed by addition of acetic acid (0.2 mL, 3.5 mmol, 3.5 eq.) under an atmosphere of nitrogen. The reaction mixture was stirred at 70° C. for 1 h (heating mantle). Sodium cyanoborohydride (55 mg, 0.8 mmol, 2.0 eq.) was added portion wise at rt, and the reaction mixture was stirred at rt overnight. Upon completion, the reaction mixture was quenched with 20 mL of saturated aqueous NaHCO₃ solution and extracted with 3×20 mL of EtOAc. The combined organic extracts were washed with 50 mL of brine and dried over anhydrous sodium sulfate. After filtration, the filtrate was concentrated by rotary evaporation under reduced pressure. The residue was purified by flash column chromatography (5% MeOH in DCM) to afford tert-butyl (R)-3-(3-((6-(4-hydroxybenzo[b]thiophen-5-yl)-5-methylpyridazin-3-yl)amino)piperidin-1-yl)azetidine-1-carboxylate (145 mg, 66%) as a yellow solid after evaporation of the combined product containing fractions under reduced pressure using a rotary evaporator. LC/MS: mass calcd. for C₂₆H₃₃N₅O₃S 495.23, found m/z=496.20 [M+H]⁺. ¹H NMR (400 MHz, Methanol-d₄): δ 7.59 (d, J=5.9 Hz, 1H), 7.49 (d, J=6.7 Hz, 2H), 7.25 (d, J=8.2 Hz, 1H), 6.85 (s, 1H), 4.52-4.47 (m, 1H), 4.31 (d, J=9.9 Hz, 1H), 3.89-3.78 (m, 2H), 3.74-3.68 (m, 2H), 3.19-3.11 (m, 1H), 3.01-2.91 (m, 1H), 2.66-2.53 (m, 1H), 2.20 (s, 3H), 2.05-1.95 (m, 2H), 1.88-1.79 (m, 1H), 1.76-1.67 (m, 1H), 1.45 (s, 9H) ppm.

### Step 2: Synthesis of (R)-5-(6-((1-(azetidin-3-yl)piperidin-3-yl)amino)-4-methylpyridazin-3-yl)benzo[b]thiophen-4-ol (114)

In a single-necked flask, tert-butyl (R)-3-(3-((6-(4-hydroxybenzo[b]thiophen-5-yl)-5-methylpyridazin-3-yl)amino)piperidin-1-yl)azetidine-1-carboxylate (145 mg, 0.3 mmol, 1.0 eq.) was dissolved in HCl/1,4-/dioxane (10 mL, 2 M, 20 mmol, 67 eq.) and the reaction mixture was stirred at rt for 1 h. Upon completion, the reaction mixture was concentrated by rotary evaporation under reduced pressure. The residue was purified by preparative RP-HPLC (5-65% ACN in H₂O, 0.1% HCOOH) to afford the target compound (R)-5-(6-((1-(azetidin-3-yl)piperidin-3-yl)amino)-4-methylpyridazin-3-yl)benzo[b]thiophen-4-ol (114) (25 mg, 22%) as a yellow solid after lyophilization of the combined product containing fractions. LC/MS: mass calcd for C₂₅H₂₅N₅OS 395.18, found m/z=396.10 [M+H]⁺. ¹H NMR (400 MHz, Methanol-d₄): δ 7.59 (d, J=5.7 Hz, 1H), 7.50 (d, J=7.2 Hz, 2H), 7.24 (d, J=8.2 Hz, 1H), 6.83 (s, 1H), 4.16-4.02 (m, 4H), 4.01-3.95 (m, 1H), 3.44-3.36 (m, 1H), 3.05 (d, J=9.3 Hz, 1H), 2.64 (d, J=12.0 Hz, 1H), 2.20 (s, 3H), 2.16-2.06 (m, 1H), 2.05-1.91 (m, 2H), 1.90-1.82 (m, 1H), 1.77-1.65 (m, 1H), 1.56-1.42 (m, 1H) ppm.

### Example 115: Determination of IL-1ß Release Inhibition in U937 Cells

The compounds of the present disclosure were tested for their ability to inhibit the IL-1ß release in U937 cells after NLRP3 activation. U937 cells (ATCC U-97 CRL 1593.2) were cultured following ATCC's recommendations. The growth medium was composed of RPMI1640 medium (Quality Biological 112-040-101) containing 10% serum (Corning 35-076-CV), Pen/Strep (GIBCO 15140-122), 10 mM HEPES buffer (Quality Biological 118-089-721), and 2 mM Glutamax (GIBCO 35050-061). Differentiating media was prepared by adding 20 nM phorbol 12-myristate 13-acetate (PMA) (AdipoGen AG-CN2-0010-M005) to growth medium. A cell suspension was prepared with differentiating media at 500K cells/mL. 200 μL of cell suspension was added to each well of a96 well plate (Corning 3903). Plates were placed in an incubator for 90 hours. Priming media was prepared with assay medium consisting of RPMI 1640 medium, 10 mM HEPES buffer, 2 mM glutamine (Quality Biologics 118-084-721) and Pen/Strep plus 1 μg/mL lipopolysaccharide (LPS) (Invivogen tlrl-3pelps). Differentiating media was removed and priming media was added to each well. The plates were placed in the incubator for three (3) hours. 20 mM compound DMSO stock solutions were prepared and were further diluted to 0.4 mM; This solution was serially diluted 6 times in a 1:5 ratio. A solvent control was included.

After three (3) hours of priming, all the media was removed. Next, assay media with compounds was added in duplicate columns. The plate was placed in an incubator for 30 min. Media with a final concentration of 10 mM nigericin was then added for one (1) hour. The supernatant was collected and frozen immediately at −80° C. The plate was transferred to −20° C. the next day and for later determination of cytokine IL-1ß levels.

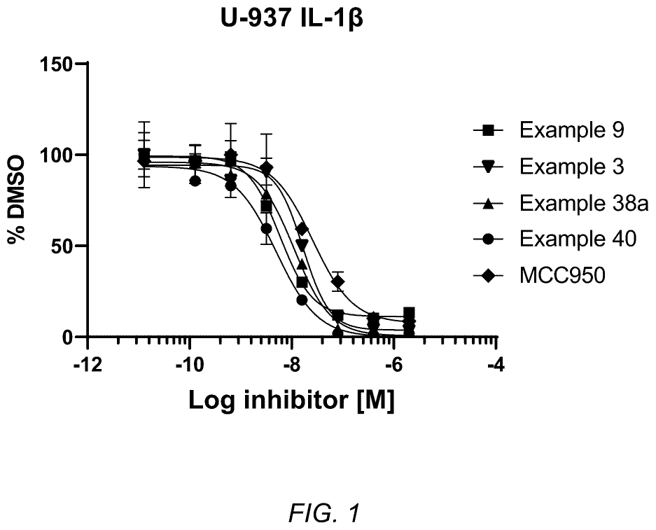

The release of IL-1ß into the cell media was determined with the MSD® Multi-Spot Assay System (V-Plex, IL-1ß kit, K151KPD). Following the manufacturer's protocol (Alternate Protocol 3, Dilute-In-Plate), the level of IL-1ß in the above collected supernatant plate was measured using electro-chemiluminescent signals. Using MSD DISCOVERY WORKBENCH® analysis software, the signals were converted to the concentration of IL-1ß in each well. The IC₅₀ values of the compounds were calculated using fitted non-linear regression curve in Graph Pad Prism software. The detection range of IL-1ß using MSD kit was approximately 0.15 to 600 μg/mL (based on various lots of standards purchased from MSD). The IC₅₀ values of the compounds of the present disclosure are summarized in Table 3 and Table 4, (“++++”: IC₅₀≤5 nM; “+++”: 5 nM<IC₅₀≤50 nM; “++”: 50 nM<IC₅₀≤500 nM; “+”: 500 nM<IC₅₀). MCC950, a compound described in the literature, was tested in each run as a positive control and demonstrated an average IC₅₀ value of 29.0±6.2 nM (N=54). Example IC₅₀ curves are shown in FIG. 1.

TABLE 3
IL-1β Release Inhibition in U937 Cells

| Example | IL-1β IC₅₀ |
|---|---|
| 1 | +++ |
| 2 | + |
| 3 | + |
| 4 | + |
| 5 | ++++ |
| 6 | ++ |
| 7 | +++ |
| 8 | +++ |
| 9 | +++ |
| 10 | +++ |
| 11 | ++++ |
| 12 | ++++ |
| 13 | +++ |
| 14 | +++ |
| 15 | ++++ |
| 16 | +++ |
| 17 | ++++ |
| 18 | +++ |
| 19 | ++ |
| 20 | ++ |
| 21 | N/A |
| 22 | +++ |
| 23 | ++++ |
| 24 | +++ |
| 25 | +++ |
| 26 | +++ |
| 27 | +++ |
| 28 | +++ |
| 29 | +++ |
| 30 | +++ |
| 31 | ++ |
| 32 | +++ |
| 33 | ++++ |
| 34 | N/A |
| 35 | +++ |
| 36 | +++ |
| 37 | +++ |
| 38A | +++ |
| 38B | +++ |
| 39A | +++ |
| 39B | +++ |
| 40 | ++++ |
| 41 | +++ |
| 42 | N/A |
| 43 | ++ |
| 44 | ++ |
| 45 | ++ |
| 46 | ++++ |
| 47 | ++ |
| 48 | +++ |
| 49 | ++++ |
| 50 | ++++ |
| 51 | ++++ |
| 52 | ++++ |
| 53 | ++++ |
| 54 | ++++ |
| 55 | ++++ |
| 56 | +++ |
| 57 | ++ |
| 58 | +++ |
| 59 | ++++ |
| 60 | ++ |
| 61 | +++ |
| 62 | ++++ |
| 63 | +++ |
| 64 | ++++ |
| 65 | Fraction A: +++ |
| | Fraction B: +++ |
| 66 | ++++ |
| 67 | +++ |
| 68 | +++ |
| 69 | ++++ |
| 70 | Fraction A: +++ |
| | Fraction B: ++++ |
| 71 | N/A |
| 72 | ++++ |
| 73 | ++++ |
| 74 | ++++ |
| 75 | ++++ |
| 76 | N/A |

TABLE 4
IL-1β Release Inhibition in U937 Cells

| Example | IL-1β IC₅₀ |
|---|---|
| 77 | +++ |
| 78 | ++++ |
| 79 | ++ |
| 80 | +++ |
| 81 | ++ |
| 82 | +++ |
| 83 | ++ |
| 84 | +++ |
| 85 | +++ |
| 86 | +++ |
| 87 | ++ |
| 88 | +++ |
| 89 | +++ |
| 90 | +++ |
| 91 | +++ |
| 92 | +++ |
| 93 | ++ |
| 94 | + |
| 95 | ++ |
| 96 | +++ |
| 97 | +++ |
| 98 | ++ |
| 99 | ++++ |
| 100 | +++ |
| 101 | +++ |
| 102 | ++ |
| 103 | +++ |
| 104 | +++ |
| 105 | N/A |
| 106 | +++ |
| 107 | +++ |
| 108 | +++ |
| 109 | +++ |
| 110 | +++ |
| 111 | +++ |
| 112 | +++ |
| 113 | ++++ |
| 114 | ++++ |

### Example 116: Determination of IL-1ß Release Inhibition in THP-1 Cells

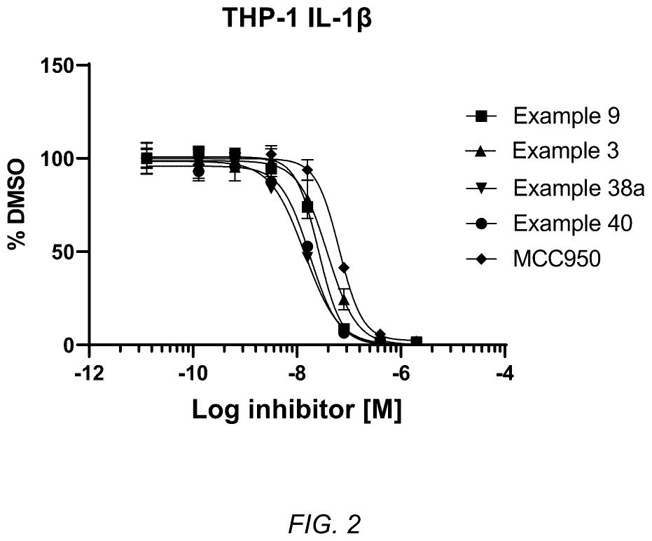

To additionally characterize the compounds as NLRP3 inhibitors, an additional monocyte cell line was used to examine effects on IL-1B and other molecules in the NLRP3 pathway. THP-1 ASC GFP cells (Invivogen thp-ascgfp) were seeded at 100K cells per well in RPMI medium supplemented with Glutamax, 10 mM HEPES, Pen/Strep and 2% FBS for overnight differentiation with 20 nM PMA. After 16 h, cells were primed with 0.1 μg/mL LPS. After 3 h, cells were treated with serial dilutions of compounds in fresh media for 30 minutes followed by the addition of nigericin to a final concentration of 10 μM for 1 hour. Supernatants were stored at −80° C. prior to analysis. Assays, calculations, and reagent vendors were as described for U937 cells. The compounds inhibited IL-1ß release in THP-1 cell similar to U937 cells. Example IC₅₀ curves are shown in FIG. 2.

### Example 117: Determination of Caspase-1 Inhibition and Viability in THP-1 Cells

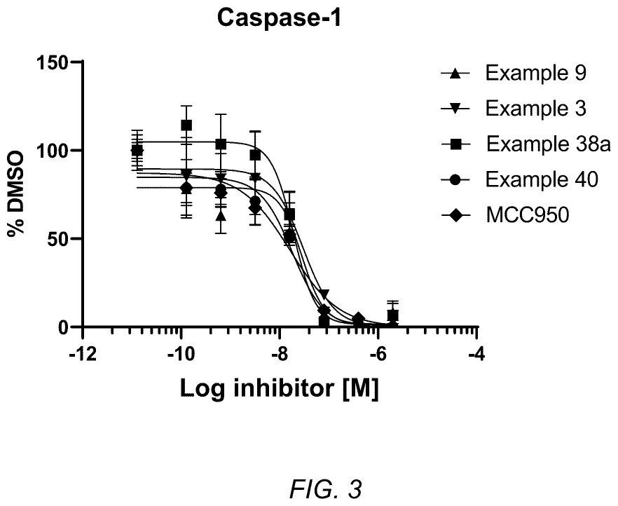

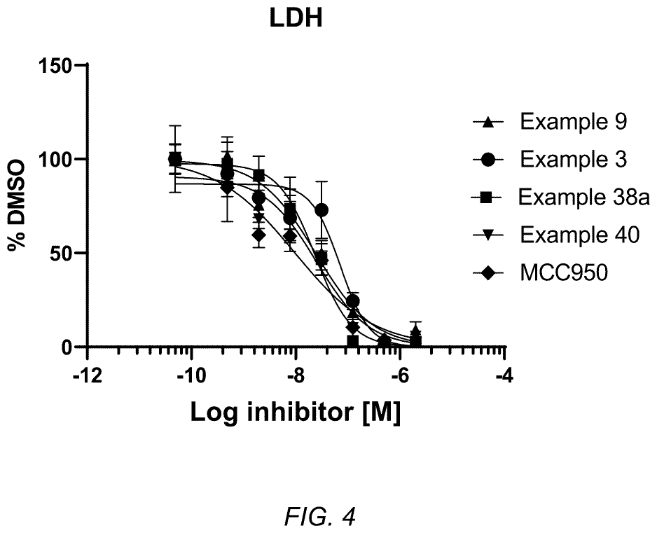

To test if compounds inhibit other parts of the NLRP3 pathway, supernatants collected in THP-1 IL-1ß assays (above) were assayed for caspase-1 activity and viability. The Promega Caspase-Glo 1 Inflammasome Assay (G9951) and LDH-Glo Cytotoxicity Assay (J2380), respectively, were conducted according to the manufacturer's recommendations. These results are additional support for NLRP3 inhibition by the compounds. The results are shown in FIG. 3 and FIG. 4.

### Example 118: Determination of ASC-GFP Speck Inhibition in THP-1 Cells

Upon activation, multiple protein molecules including NLRP3 and ASC come together to form the macromolecular inflammasome complex. To monitor this formation a reporter cell line was used that expresses an ASC-GFP (Green Fluorescent Protein) fusion protein where GFP specks reflect inflammasome formation. Compounds were tested for inhibition in this assay to additionally support their characterization as NLRP3 inhibitors.

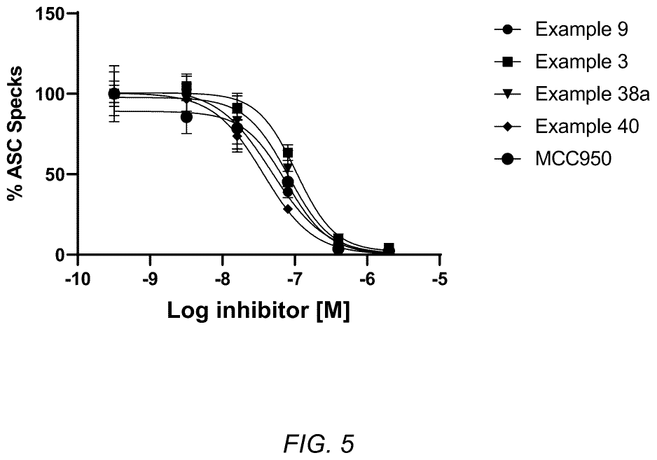

THP-1 ASC GFP cells were seeded in 96-well plates at 100K cells per well in RPMI with Glutamax, 10 mM HEPES, Pen/Strep, and 10% FBS (growth media) for 72 h differentiation with 20 nM PMA. Fresh growth media was added for 24 h prior to 3 h treatment with 1 μg/mL LPS in media without FBS. Next, the cells were treated with serial dilutions of compounds in fresh media for 30 minutes prior to the addition of nigericin to a final concentration of 10 μM for 1 hour. Cells were then fixed in formalin for 10 minutes. Nuclei were stained with NucBlue (Invitrogen R37606) in TBS (Tris Buffered Saline VWR J640). Images were acquired from 5 wells per treatment using a Nikon Ti2-E eclipse inverted microscope. ASC GFP specks and nuclei were quantified with Nikon AR Elements software. Potency calculations were performed as described for U937 cells. The results are shown in FIG. 5.

### Example 119: Compounds have Minimal Effects on TNFα in U937 Cells

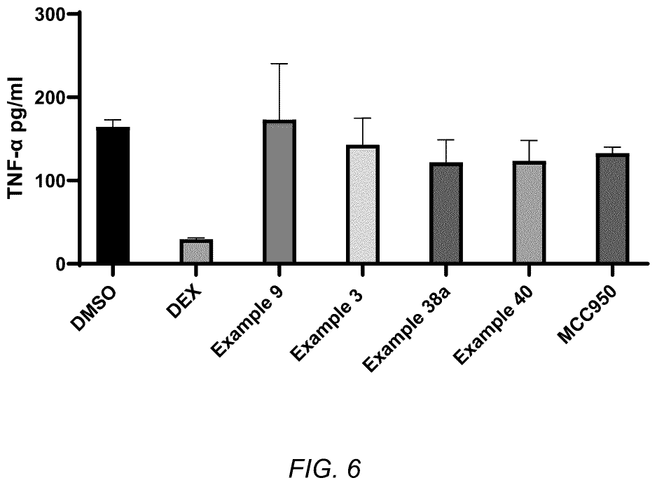

NLRP3 specific inhibitors are not expected to have broad anti-inflammatory effects such as modulation of TNFα levels. To test for this potential off-target activity, U937 cells were differentiated as above for 92 h followed by 3 h 1 μg/mL LPS (Invivogen) stimulation. Cells were next treated with 1 μM compound or dexamethasone (DEX) as a positive control for 22 h. TNFα levels in the cell supernatants were determined using MesoScale Discovery kit (V-Plex, TNFα kit, K151QW) following the manufacturer's recommendations (Alternate Protocol 3, Dilute-In-Plate). Calculations were performed as described for IL-1B determination above. The results are shown in FIG. 6.

### Example 120: Toxicity Assessment in Human Retinal Pigment Epithelial Cells

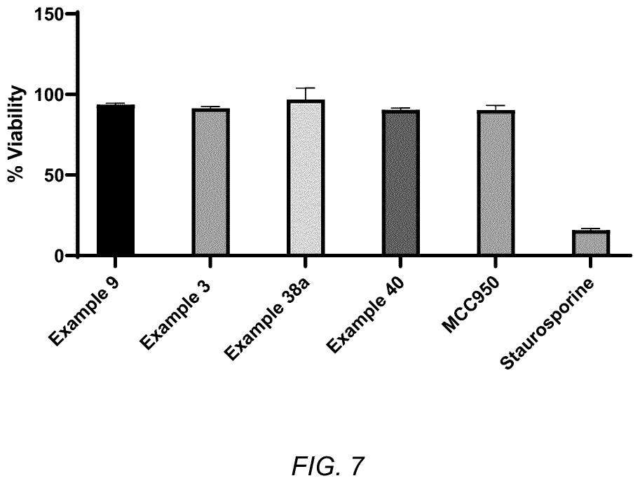

ARPE-19 cells, a human retinal pigment epithelial cell line, were seeded at 7.5K cells/well in 96 well plates overnight in DMEM/F12 (GIBCO 21041-025) supplemented with 10% FBS Pen/Strep. Cells were treated with serial dilutions of compounds for 72 h in the same media as well as 5 μM staurosporine as a positive control. Toxicity was assessed using alamar blue (Fisher Scientific DAL1100) according to the manufacturer's protocol. Toxicity was not observed at or below 30 μM. The results are shown in FIG. 7.

What is claimed is:

1. A compound represented by the structure of Formula (1-I): 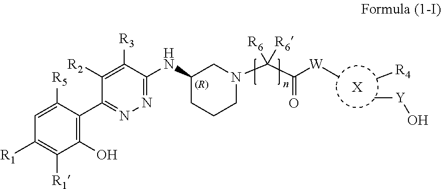
or a pharmaceutically acceptable salt, solvate, isomer, atropisomer, or tautomer thereof, wherein
ring 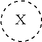
is a 4-11 membered cycloalkyl, heterocycloalkyl, substituted cycloalkyl, or substituted heterocyloalkyl ring;
W is selected from the group consisting of NH, N-Me, N-Et and N-iPr, or is a bond;
R₁ is selected from the group consisting of —CN, —H, —F, and —CF₃;
R₁′ is H;
or R₁′ together with R₁ form a thiophene ring;
R₂ is selected from the group consisting of H, C1-C3 alkyl, and —CF₃;
R₃ is selected from the group consisting of H, C1-C3 alkyl, and —CF₃;
or R₃ together with R₂ form a 5- or 6-membered aromatic or non-aromatic ring, all carbon or with O or N within the ring, optionally substituted with -Me or —OH;
R₄ is selected from the group consisting of H, —F, C1-C3 alkyl, and —CF₃;
R₅ is selected from the group consisting of H, Me, and —CF₃;
R₆ and R₆′ are independently selected from the group consisting of —H, —F, C1-C6 alkyl, C1-C6 cycloalkyl, and —CF₃;
or R₆ and R₆′ together with the atom they are bonded to form a C1-C6 cycloalkyl or C1-C6 heterocycloalkyl ring;
Y is a bond or C1-C3 alkyl optionally substituted with 1, 2, or 3 C1-C3 alkyl or —CF₃; and
n is 1, 2, 3, or 4.

2. The compound of claim 1, wherein 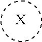
is selected from the group consisting of cyclobutane, cyclopentane, cyclohexane, azetidine, pyrrolidine, piperidine, 2-azaspiro[3.3]heptane, 2-azaspiro[3.5]nonane, 7-azaspiro[3.5]nonane, spiro[3.3]heptane, and bicyclo[1.1.1]pentane.

3. The compound of claim 2 wherein 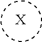
is selected from the group consisting of 2-azaspiro[3.3]heptane, 2-azaspiro[3.5]nonane and 7-azaspiro[3.5]nonane, W is a bond, and Y is a bond, and R₄ is H.

4. The compound of claims 1-3 wherein R₁ is —CF₃; R₂ is methyl; R₃ is H or methyl; or R₂ and R₃ form a 5-membered non-aromatic ring, all carbon or with O in the ring; R₅ is H.

5. The compound of claim 1, further represented by any one of the following:

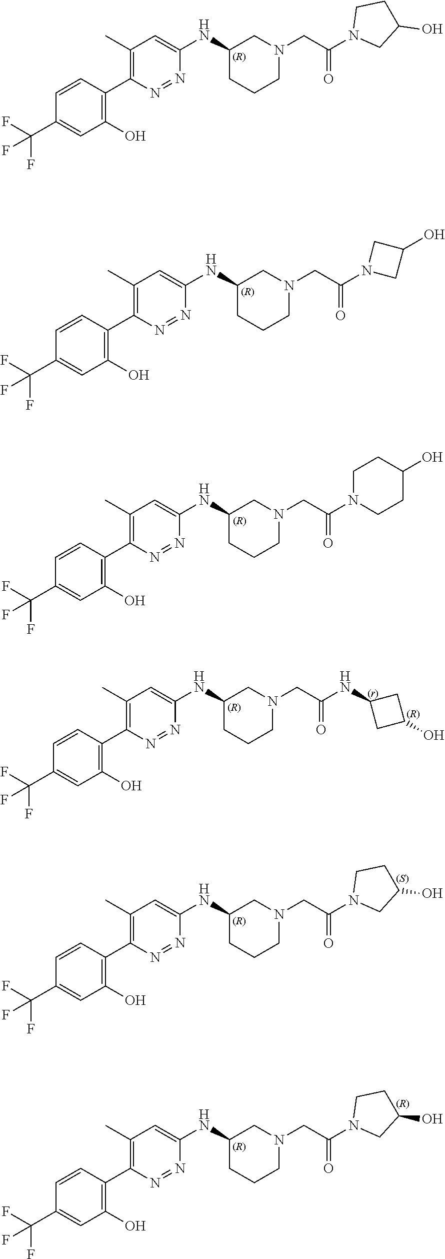

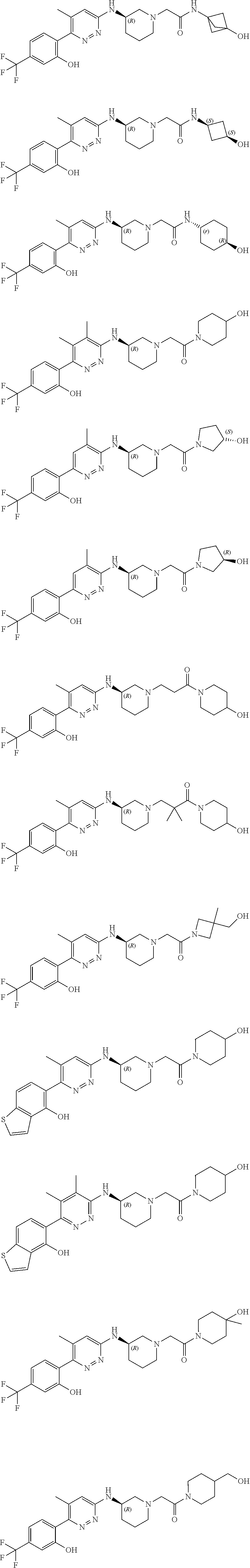

6. The compound of claim 1, further represented by any one of the following:

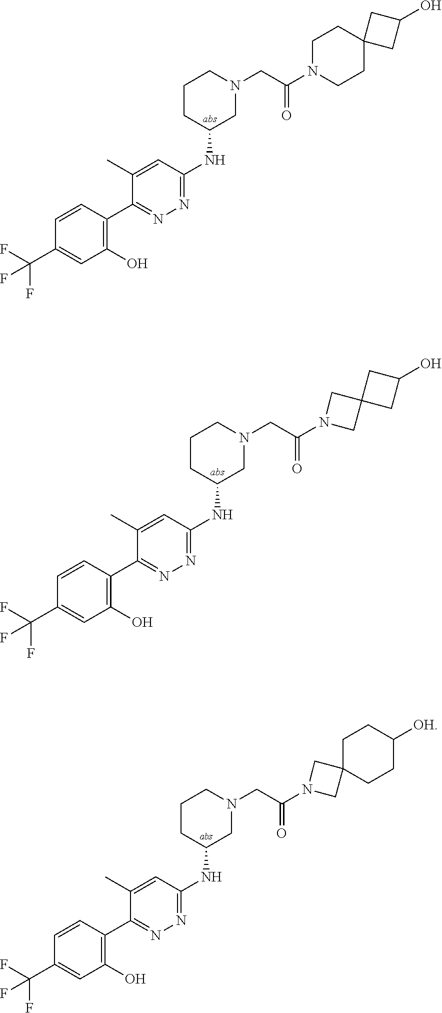 [SMILES: Cc1cc(N[C@@H]2CCCN(CC(=O)N3CCC4(CC3)CC(O)C4)C2)nnc1-c1ccc(C(F)(F)F)cc1O ; Cc1cc(N[C@@H]2CCCN(CC(=O)N3CC4(CC(O)C4)C3)C2)nnc1-c1ccc(C(F)(F)F)cc1O ; Cc1cc(N[C@@H]2CCCN(CC(=O)N3CC4(CCC(O)CC4)C3)C2)nnc1-c1ccc(C(F)(F)F)cc1O]

7. The compound of claim 1, further represented by any one of the following:

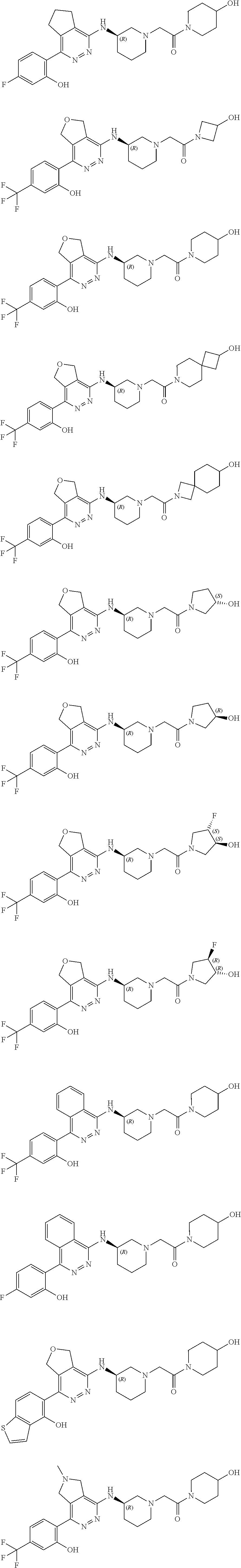

8. A compound represented by the structure of Formula (1-II): 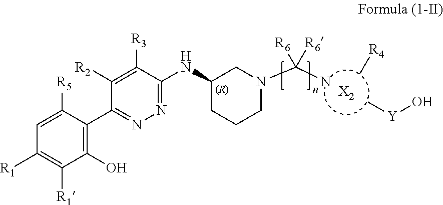
or a pharmaceutically acceptable salt, solvate, isomer, atropisomer, or tautomer thereof;
wherein ring 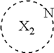
is a 4-11 membered nitrogen-containing heterocycloalkyl ring or substituted heterocycloalkyl ring;
R₁ is selected from the group consisting of —CN, —H, —F, and —CF₃;
R₁′ is H;
or R₁ together with R₁′ form a thiophene ring;
R₂ is selected from the group consisting of H, C1-C3 alkyl, and —CF₃;
R₃ is selected from the group consisting of H, C1-C3 alkyl, and —CF₃;
or R₂ together with R₃ form a 5- or 6-membered aromatic or non-aromatic ring, all carbon or with O or N, optionally substituted with -Me or —OH;
Y is a bond or C1-C3 alkyl optionally substituted with 1, 2, or 3 C1-C3 alkyl or —CF₃;
R₄ is selected from the group consisting of H, —F, C1-C3 alkyl, and —CF₃;
R₅ is selected from the group consisting of H, Me, and —CF₃;
R₆ and R₆′ are independently selected from the group consisting of —H, —F, C₁-C₆ alkyl, C1-C6 cycloalkyl, and —CF₃;
or R₆ and R₆′ together with the atom they are bonded to form a C1-C6 cycloalkyl or C1-C6 heterocycloalkyl ring; and
n is 2, 3, or 4.

9. A compound of claim 8 wherein ring 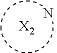
is selected from pyrrolidine, piperidine and azetidine; R₁ is —CF₃; R₂ is methyl; R₃ is H or methyl; or R₂ and R₃ form a 5-membered non-aromatic ring, all carbon or with O in the ring; R₄ is H or F; Y is a bond; and R₅ is H;

10. A compound of claim 8 further represented by any one of the following:

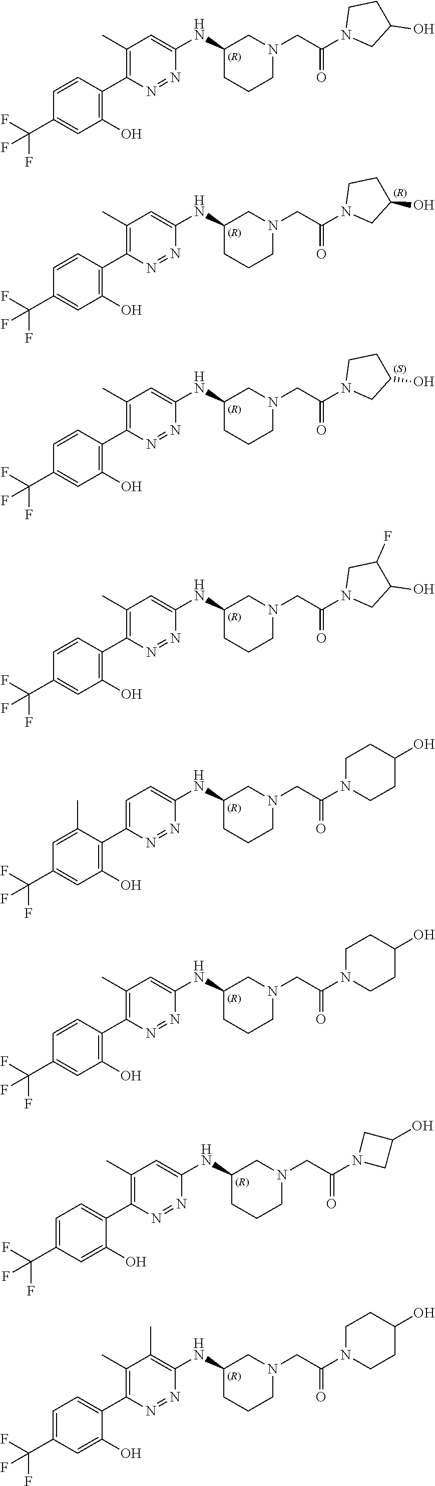

11. A compound of claim 8 further represented by any one of the following:

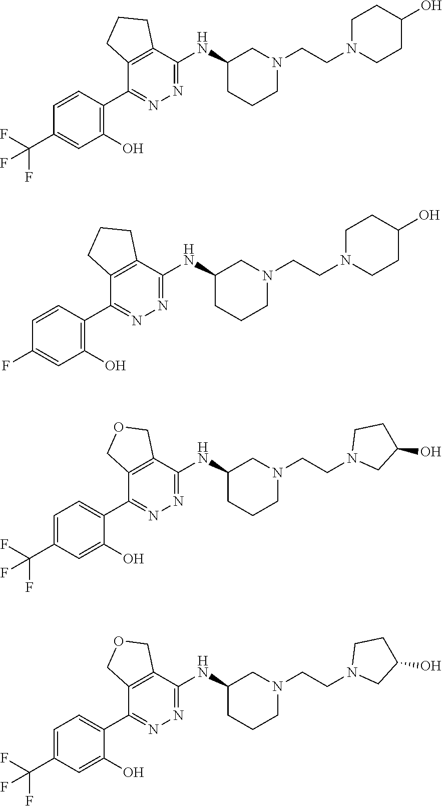 [SMILES: Oc1cc(C(F)(F)F)ccc1-c1nnc(N[C@@H]2CCCN(CCN3CCC(O)CC3)C2)c2c1CCC2 ; Oc1cc(F)ccc1-c1nnc(N[C@@H]2CCCN(CCN3CCC(O)CC3)C2)c2c1CCC2 ; Oc1cc(C(F)(F)F)ccc1-c1nnc(N[C@@H]2CCCN(CCN3CC[C@@H](O)C3)C2)c2c1COC2 ; Oc1cc(C(F)(F)F)ccc1-c1nnc(N[C@@H]2CCCN(CCN3CC[C@H](O)C3)C2)c2c1COC2]

12. A compound represented by the structure of Formula (1-III): 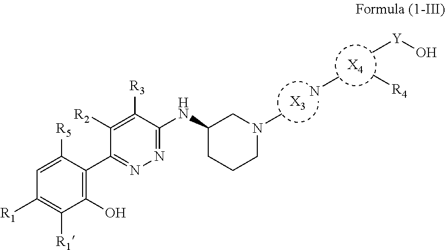
or a pharmaceutically acceptable salt, solvate, isomer, atropisomer, or tautomer thereof,
wherein ring 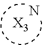
is a 4-7 membered nitrogen-containing heterocycloalkyl ring or substituted heterocycloalkyl ring;
ring 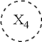
is a 4-7 membered cycloalkyl ring or substituted cycloalkyl ring;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, and —CF₃;
R₃ is selected from the group consisting of H, C1-C3 alkyl, and —CF₃;
or R₂ together with R₃ form a 5- or 6-membered aromatic or non-aromatic ring, all carbon or with O or N, optionally substituted with -Me or —OH;
R₄ is selected from the group consisting of H, —F, C1-C3 alkyl, and —CF₃; and
Y is a bond or C1-C3 alkyl optionally substituted with 1, 2, or 3 C1-C3 alkyl or —CF₃.

13. A compound of claim 12 wherein ring 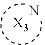
is piperidine, pyrrolidine or azetidine; ring 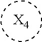
is cyclobutane, cyclopentane or cyclohexane; R₁ is —CF₃; R₂ is methyl; R₃ is H or methyl; or R₂ and R₃ form a 5-membered non-aromatic ring, all carbon or with O in the ring; R₄ is H; Y is a bond.

14. A compound of claim 12 further represented by any one of the following:

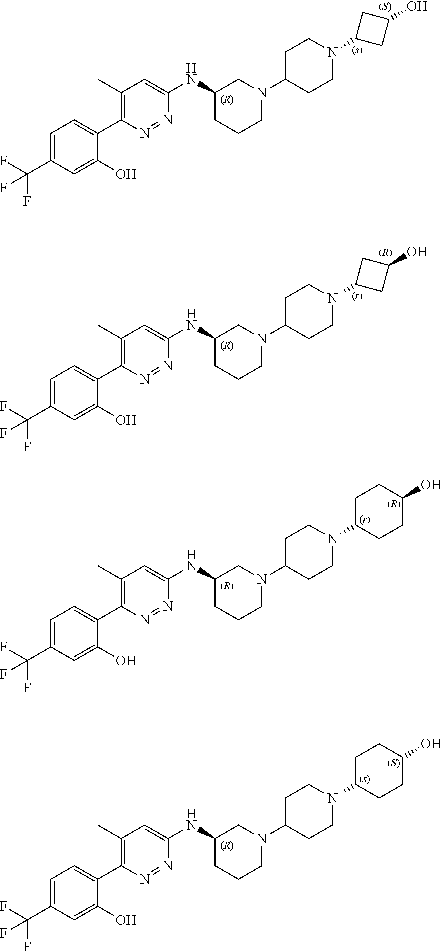

15. A compound represented by the structure of Formula (1-IV): 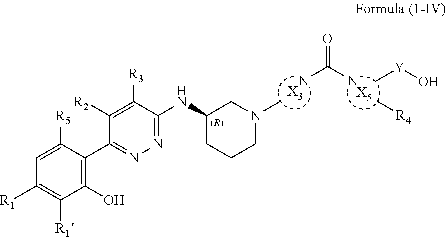
or a pharmaceutically acceptable salt, solvate, isomer, atropisomer, or tautomer thereof,
wherein ring 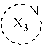
is a 4-7 membered nitrogen-containing heterocycloalkyl ring or substituted heterocycloalkyl ring;
wherein ring 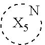
is a 4-7 membered nitrogen-containing heterocycloalkyl ring or substituted hetereocycloalkyl ring;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, and —CF₃;
R₃ is selected from the group consisting of H, C1-C3 alkyl, and —CF₃;
or R₂ together with R₃ form a 5- or 6-membered aromatic or non-aromatic ring, all carbon or with O or N within the ring, optionally substituted with -Me or —OH;
Y is a bond or C1-C3 alkyl optionally substituted with 1, 2, or 3 C1-C3 alkyl or —CF₃;
R₄ is selected from the group consisting of H, —F, C1-C3 alkyl, and —CF₃; and
R₅ is selected from the group consisting of —H, -Me and —CF₃.

16. A compound of claim 15 wherein ring 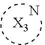
is selected from azetidine, pyrrolidine and piperidine; ring 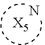
is selected from the group consisting of azetidine, pyrrolidine and piperidine; R₁ is —CF₃; R₂ is methyl; R₃ is H or methyl; or R₂ and R₃ form a 5-membered non-aromatic ring, all carbon or with O in the ring; R4 is H; Y is a bond.

17. A compound of claim 15 further represented by any one of the following:

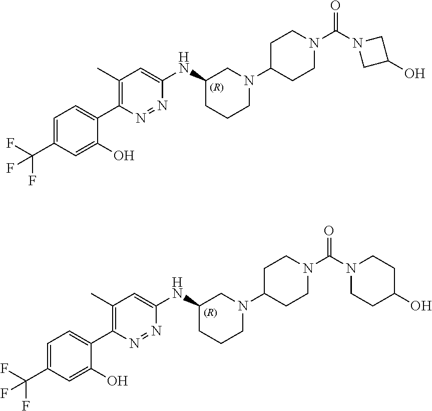

18. A compound represented by the structure of Formula (1-V): 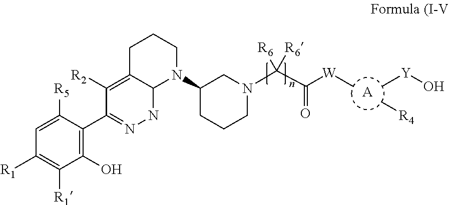
or a pharmaceutically acceptable salt, solvate, isomer, atropisomer, or tautomer thereof,
wherein ring 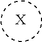
is a 4-11 membered cycloalkyl heterocycloalkyl, substituted cycloalkyl, or substituted heterocycloalkyl ring; W is NH, N-Me, N-Et, N-iPr, or a bond;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, —CF₃;
R₄ is selected from the group consisting of H, —F, C1-C3 alkyl, and —CF₃;
Y is a bond or C1-C3 alkyl optionally substituted with 1, 2, or 3 C1-C3 alkyl or —CF₃;
R₅ is selected from the group consisting of —H, -Me, and —CF₃;
R₆ and R₆′ are independently selected from the group consisting of —H, —F, C1-C6 alkyl, and —CF₃,
or R₆ and R₆′ together with the atom they are bonded to form a C1-C6 cycloalkyl or C1-C6 heterocycloalkyl ring; and
n is 1, 2, 3, or 4.

19. The compound of claim 18 wherein 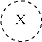
is selected from the group consisting of cyclobutane, cyclopentane, cyclohexane, azetidine, pyrrolidine, piperidine, 2-azaspiro[3.3]heptane, 2-azaspiro[3.5]nonane, 7-azaspiro[3.5]nonane, spiro[3.3]heptane, and bicyclo[1.1.1]pentane.

20. The compound of claim 19 wherein 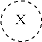
is selected from the group consisting of 2-azaspiro[3.3]heptane, 2-azaspiro[3.5]nonane and 7-azaspiro[3.5]nonane; W is a bond; R1 is —CF₃; R₂ is methyl; and R₄ is H; and Y is a bond.

21. A compound of claim 18 further represented by any one of the following:

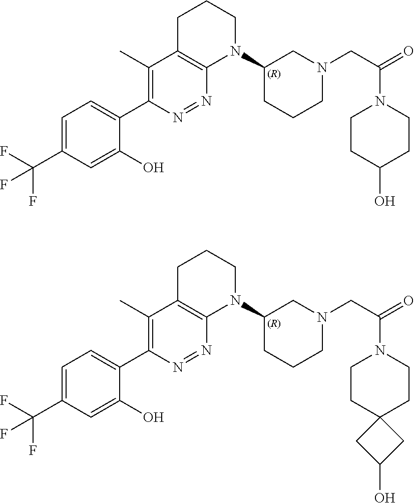

22. A compound represented by the structure of Formula (1-VI): 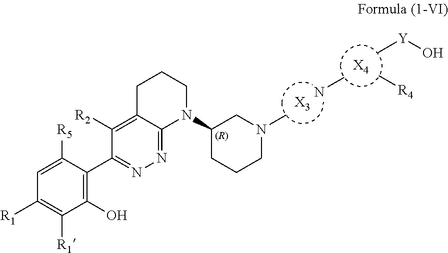
wherein ring 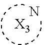
is an optionally substituted 4-7 membered nitrogen-containing heterocycloalkyl ring or substituted heterocycloalkyl ring;
ring 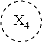
is an optionally substituted 4-7 membered cycloalkyl ring or substituted cycloalkyl ring;
R₁ is selected from the group consisting of —CN, —H, —F, and —CF₃;
R₁′ is H;
or R₁ and R₁′ together form a thiophene ring;
R₂ is selected from the group consisting of —H, C1-C3 alkyl, and —CF₃;
R₄ is selected from the group consisting of —H, —F, C1-C3 alkyl, and —CF₃;
R₅ is selected from the group consisting of —H, -Me, and —CF₃; and
Y is a bond or C1-C3 alkyl optionally substituted with 1, 2, or 3 C1-C3 alkyl or —CF₃.

23. A compound of claim 22 further represented by any one of the following:

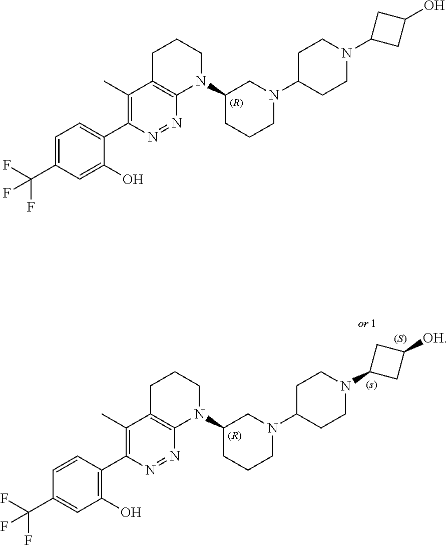

24. A compound represented by the structure of Formula (1-VII): 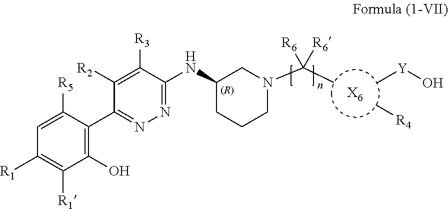
wherein ring 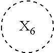
is a 4-11 membered cycloalkyl heterocycloalkyl, substituted cycloalkyl, or substituted heterocycloalkyl ring;
R₁ is selected from the group consisting of —CN, —H, —F, and —CF₃;
R₁′ is H;
or R₁ and R₁′ together form a thiophene ring;
R₂ is selected from the group consisting of —H, C1-C3 alkyl, —CF₃;
R₄ is selected from the group consisting of —H, —F, C1-C3 alkyl, and —CF₃;
R₅ is selected from the group consisting of —H, -Me, and —CF₃;
R₆ and R₆′ are independently selected from the group consisting of —H, —F, C1-C6 alkyl, C₁-C₆ cycloalkyl, and —CF₃,
or R₆ and R₆′ together with the atom they are bonded to form a C1-C6 cycloalkyl or C1-C6 heterocycloalkyl ring;
n is 0, 1, 2, 3, or 4; and
Y is a bond or C1-C3 alkyl optionally substituted with 1, 2, or 3 C1-C3 alkyl or —CF₃.

25. The compound of claim 24 further represented by any one of the following:

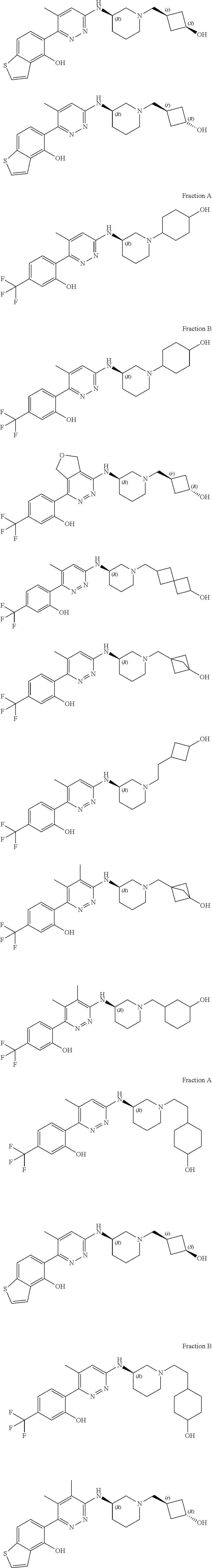

26. A compound represented by the structure of Formula (1-VIII): 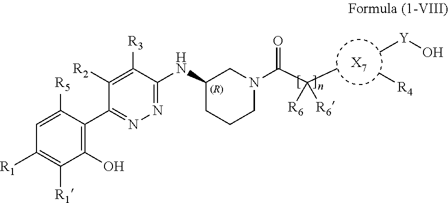
wherein ring 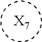
is a 4-11 membered cycloalkyl, heterocycloalkyl, substituted cycloalkyl, or substituted heterocycloalkyl ring;
R₁ is selected from the group consisting of —CN, —H, —F, and —CF₃;
R₁′ is H;
or R₁ and R₁′ together form a thiophene ring;
R₂ is selected from the group consisting of —H, C1-C3 alkyl, and —CF₃;
R₄ is selected from the group consisting of H, —F, C1-C3 alkyl, and —CF₃;
R₅ is selected from the group consisting of H, Me, and —CF₃;
R₆ and R₆′ are independently selected from the group consisting of —H, —F, C1-C6 alkyl, and —CF₃,
or R₆ and R₆′ together with the atom they are bonded to form a C1-C6 cycloalkyl or C1-C6 heterocycloalkyl ring;
n is 1, 2, 3, or 4; and
Y is a bond (or C1-C3 alkyl optionally substituted with 1, 2, or 3 C1-C3 alkyl or —CF₃.

27. A compound of claim 26 further represented by any one of the following:

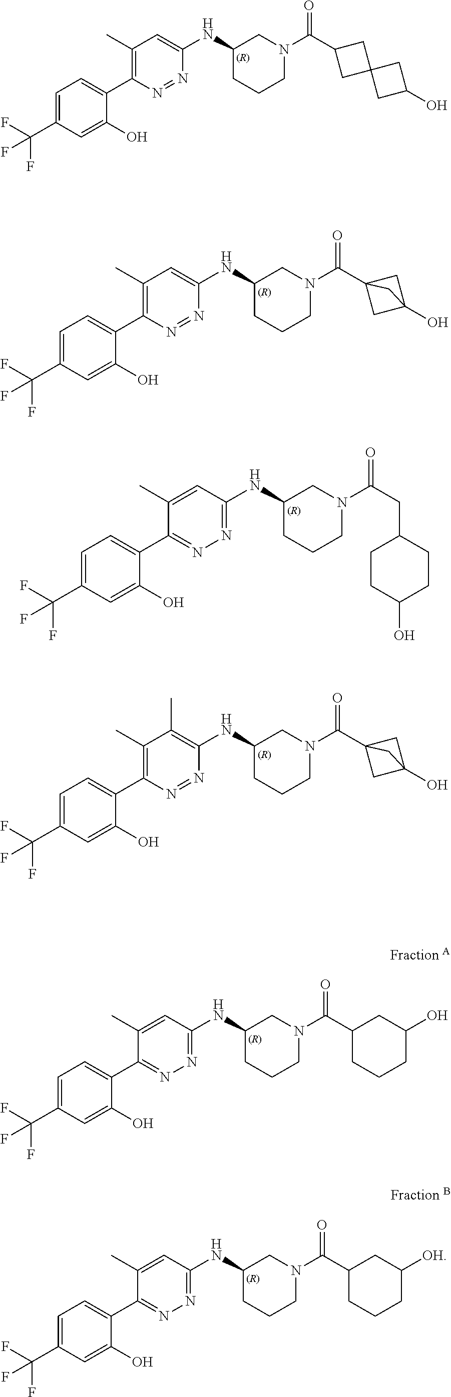

28. A compound represented by the structure of Formula (1-IX): 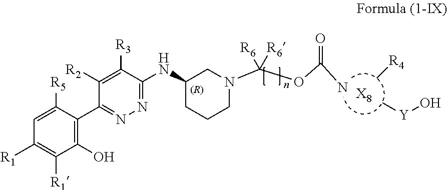
or a pharmaceutically acceptable salt, solvate, isomer, atropisomer, or tautomer thereof,
wherein ring 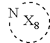
is a 4-11 membered nitrogen-containing heterocycloalkyl ring or substituted heterocycloalkyl ring,
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, —CF₃;
R₃ is selected from the group consisting of H, C1-C3 alkyl, and —CF₃;
or R₂ together with R₃ form a 5- or 6-membered aromatic or non-aromatic ring, all carbon or with O or N within the ring, optionally substituted with -Me or —OH;
R₄ is selected from the group consisting of H, —F, C1-C3 alkyl, and —CF₃;
R₅ is selected from the group consisting of H, Me, and —CF₃;
R₆ and R₆′ are independently selected from the group consisting of —H, —F, C1-C6 alkyl, C1-6 cycloalkyl, and —CF₃,
or R₆ and R₆′ together with the atom they are bonded to form a C1-C6 cycloalkyl or C1-C6 heterocycloalkyl ring;
n is 2, 3, or 4; and
Y is a bond (or C1-C3 alkyl optionally substituted with 1, 2, or 3 C1-C3 alkyl or —CF₃.

29. The compound of claim 28 wherein ring 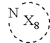
is selected from pyrrolidine, piperidine and azetidine; R₁ is —CF₃; R₂ is methyl; R₃ is H or methyl; or R₂ and R₃ form a 5-membered non-aromatic ring, all carbon or with O in the ring; R₄ is H or F; R₅ is H; Y is a bond.

30. The compound of claim 28 further represented by any one of the following:

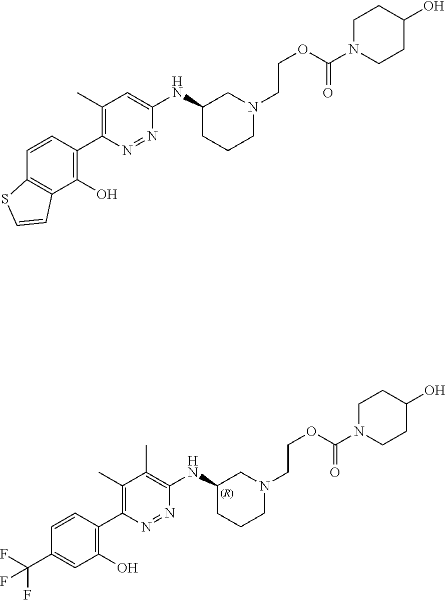

31. A compound represented by the structure of Formula (2-I) 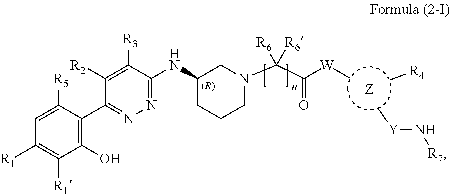
or a pharmaceutically acceptable salt, solvate, hydrate, isomer, atropisomer, or tautomer thereof,
wherein ring 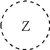
is a 4-11 membered cycloalkyl, substituted cycloalkyl, heterocycloalkyl, or substituted heterocycloalkyl ring;
W is selected from the group consisting of NH, N-Me, N-Et and N-iPr, or W is a bond;
R₁ is selected from the group consisting of —CN, —H, —F, and —CF₃;
R₁′ is —H;
or R₁ together with R₁′ form a thiophene ring;
each R₂ and R₃ is selected from the group consisting of —H, C1-C3 alkyl, and —CF₃;
or R₂ together with R₃ forms a 5- or 6-membered aromatic or non-aromatic ring containing all C or at least one O or N, optionally substituted with -Me or —OH;
R₄ is selected from the group consisting —H, —F, C1-C3 alkyl, and —CF₃;
Y is a bond or C1-C3 alkyl optionally substituted with 1, 2, or 3 C1-C3 alkyl or —CF₃.
R₅ is selected from the group consisting of —H, -Me and —CF₃;
R₆ and R₆′ are independently selected from the group consisting of —H, —F, C1-C6 alkyl or cycloalkyl, and —CF₃;
or R₆ and R₆′ together with the atom they are bonded to form a C1-C6 cycloalkyl or C1-C6 heterocycloalkyl ring;
n is 1, 2, 3, or 4; and
R₇ is selected from the group consisting of —H, C1-C6 alkyl, cycloalkyl, heterocylcoalkyl,

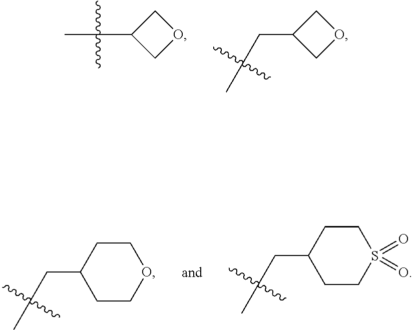 [SMILES: *C1COC1 ; *CC1COC1 ; *CC1CCOCC1 ; *CC1CCS(=O)(=O)CC1]

32. The compound of claim 31 wherein the ring 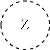
is spirocyclic or bicyclic cycloalkyl, substituted spirocyclic or bicyclic cycloalkyl, spirocyclic or bicyclic heterocycloalkyl, or substituted spirocyclic and bicyclic heterocycloalkyl.

33. The compound of claim 32 wherein W is NH, N-Me, or a bond; Y is a bond;

34. The compound of claims 31-33 wherein
R1 is —F or —CF₃;
R1′ is —H;
R2 is —H, -Me, or —CF₃;
R3 is —H or -Me;
or R3 together with R2 forms a 5- or 6-membered aromatic or non-aromatic ring containing all C or at least one O;
R4 is —H;
R5 is selected from —H and -Me;
R6 and R6′ are each —H;
n is 1; and
R7 is —H.

35. The compound of claim 31, further represented by any one of the following:

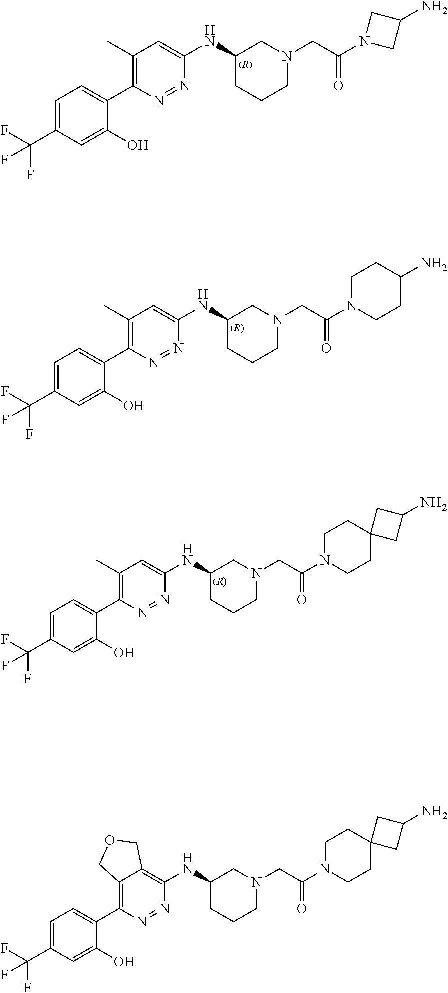

36. A compound represented by the structure of Formula (2-II) 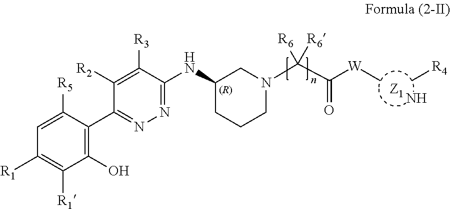
or a pharmaceutically acceptable salt, solvate, hydrate, isomer, atropisomer, or tautomer thereof,
wherein ring 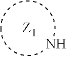
is a 4-11 membered nitrogen-containing heterocycloalkyl ring or substituted heterocycloalkyl ring;
W is selected from NH, N-Me, N-Et and N-iPr, or is a bond;
R₁ is selected from the group consisting of —CN, —H, —F, and —CF₃;
R₁′ is H;
or R₁ and R₁′ together form a thiophene ring;
R₂ and R₃ are each independently selected from the group consisting of H, C1-C3 alkyl, and —CF₃;
or R₂ together with R₃ form a 5- or 6-membered aromatic or non-aromatic ring, all carbon or with O or N within the ring, optionally substituted with -Me or —OH;
R₄ is selected from the group consisting of —H, —F, C1-C3 alkyl, and —CF₃;
R₅ is selected from the group consisting of —H, -Me, and —CF₃;
R₆ and R₆′ are independently selected from the group consisting of —H, —F, C1-C6 alkyl or cycloalkyl, and —CF₃;
or R₆ and R₆′ together with the atom they are bonded to form a C1-C6 cycloalkyl or C1-C6 heterocycloalkyl ring; and
n is 1, 2, 3, or 4.

37. The compound of claim 36 wherein ring 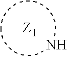
is a 4-11 membered spirocyclic or bicyclic nitrogen-containing heterocycloalkyl.

38. The compound of claim 37 wherein W is NH, N-Me, or a bond;

39. The compound of any one of claims 36-38 wherein R₁ is selected from —F or —CF₃; R₁′ is —H; R₂ is —H, -Me, or —CF₃; R₃ is selected from —H, or -Me, or R₂ and R₃ form a 5- or 6-membered aromatic or non-aromatic ring, all carbon or with O within the ring; R₄ is —H or -Me; R₅ is selected from —H and -Me; R₆ and R₆′ are —H; n is 1.

40. The compound of claim 36, further represented by any one of the following:

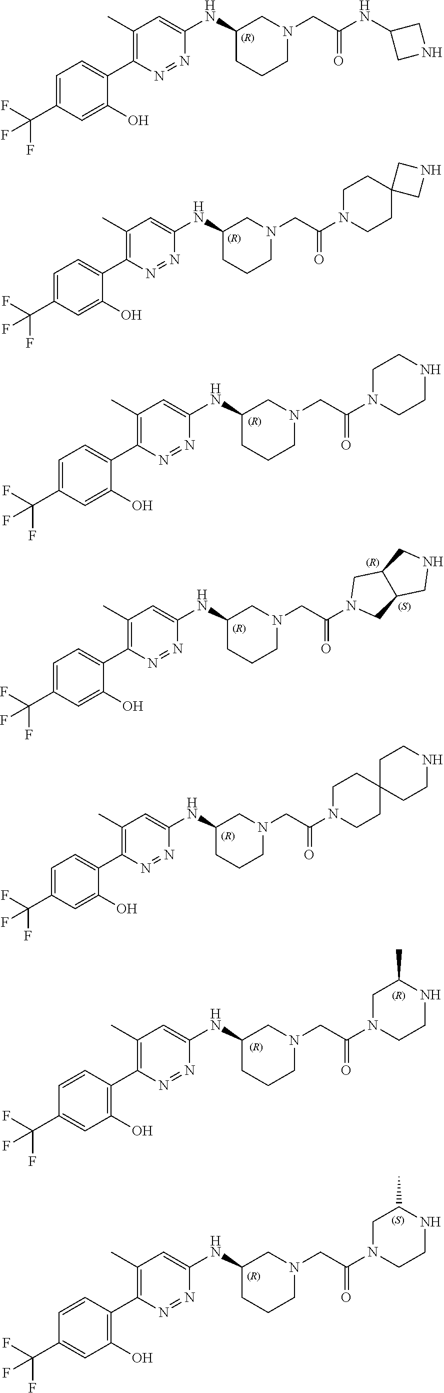

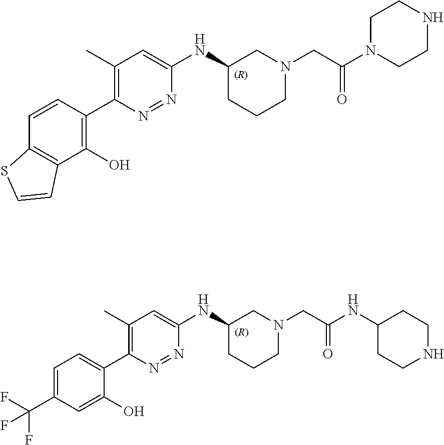

41. A compound represented by the structure of Formula (2-III) 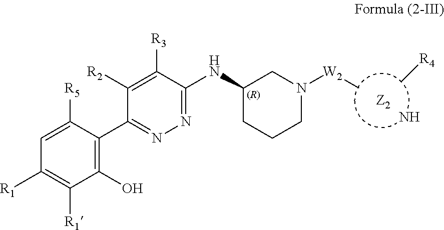
or a pharmaceutically acceptable salt, solvate, hydrate isomer, atropisomer, or tautomer thereof,
wherein ring 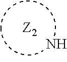
is a 4-11 membered heterocycloalkyl or substituted heterocycloalkyl ring;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, —CF₃;
R₃ is selected from the group consisting of H, C1-C3 alkyl, —CF₃;
Or R₂ together with R₃ form a 5- or 6-membered aromatic or non-aromatic ring, all carbon or with O or N within the ring, optionally substituted with -Me or —OH;
R₄ is selected from the group consisting of —H, —F, C1-C3 alkyl, and —CF₃;
R₅ is selected from the group consisting of —H, -Me and —CF₃;
W₂ is selected from the group consisting of optionally substituted —CH₂—, —CH₂CH₂—, and —CH₂CH₂CH₂—, or is a bond.

42. compound The of claim 41 wherein ring 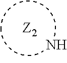
is a 4-9 membered nitrogen-containing spirocyclic or bicyclic nitrogen-containing heterocycloalkyl ring.

43. The compound of claim 42 wherein W₂ is —CH2-, —CH2CH2-, or is a bond;

44. The compound of any one of claims 41-43 wherein R₁ is —F, or —CF3; R1′ is —H; R2 is —H, -Me, or —CF3, or R₂ and R₃ form a 5- or 6-membered non-aromatic ring, all carbon or with O within the ring; R4 is —H or -Me; R5 is selected from —H and -Me.

45. The compound of claim 41, further represented by any one of the following:

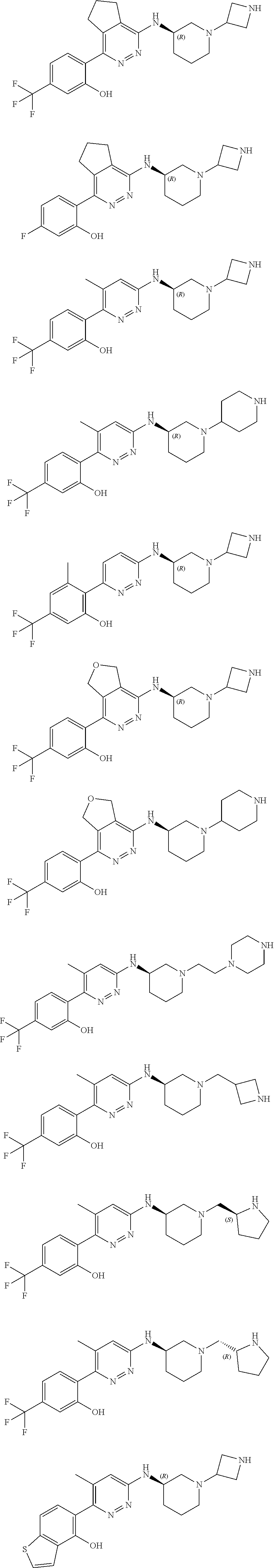

46. A compound represented by the structure of Formula (2-IV): 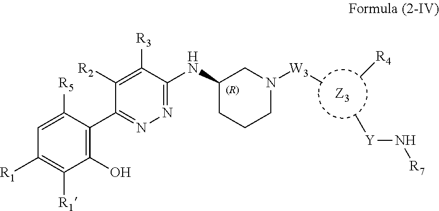
or a pharmaceutically acceptable salt, solvate, hydrate, isomer, atropisomer, or tautomer thereof, wherein:
ring 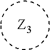
is a 4-11 membered cycloalkyl, substituted cycloalkyl, heterocycloalkyl or substituted heterocycloalkyl ring;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, —CF₃;
R₃ is selected from the group consisting of H, C1-C3 alkyl, —CF₃;
or R₂ together with R₃ form a 5- or 6-membered aromatic or non-aromatic ring, all carbon or with O or N within the ring, optionally substituted with -Me or —OH;
R₄ is selected from the group consisting of —H, —F, C1-C3 alkyl, and —CF₃;
R₅ is selected from the group consisting of —H, -Me, and —CF₃;
W₃ is selected from the group consisting of optionally substituted —CH₂—, —CH₂CH₂—, and —CH₂CH₂CH₂—, or is a bond;
Y is a bond or C1-C3 alkyl optionally substituted with 1, 2, or 3 C1-C3 alkyl or —CF₃; and
R₇ is selected from the group consisting of —H, C1-C6 alkyl, cycloalkyl, heterocycloalkyl

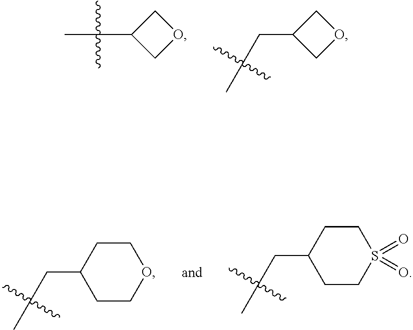 [SMILES: *C1COC1 ; *CC1COC1 ; *CC1CCOCC1 ; *CC1CCS(=O)(=O)CC1]

47. The compound of claim 46 wherein ring 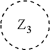
is a 4-6 membered cycloalkyl ring or heterocycloalkyl ring.

48. The compound of claim 47 wherein W3 is selected from —CH2- and —CH2CH2-, or is a bond; Y is a bond.

49. The compound of any one of claims 46-48 wherein R1 is selected from —F, —CF3; R1′ is —H; R2 is selected from —H, -Me, —CF3; R₃ is selected from —H, or -methyl, or R₂ and R₃ form a 5- or 6-membered aromatic or non-aromatic ring, all carbon or with O within the ring; R₄ is —H or -Me; R₅ is selected from —H and -Me; R₇ is —H.

50. The compound of claim 46, further represented by any one of the following:

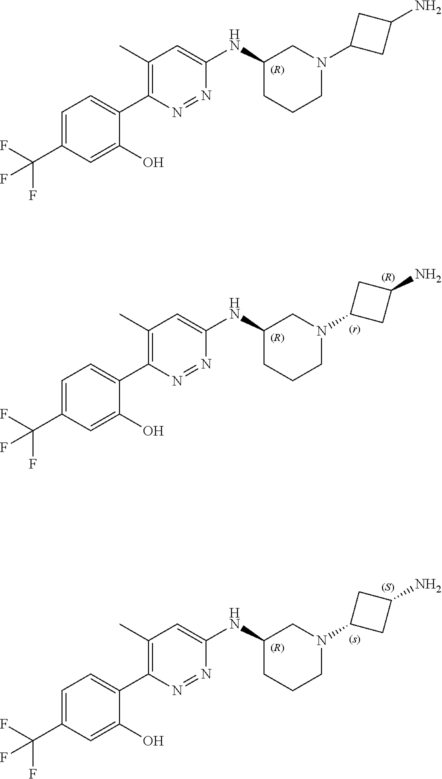

51. A compound represented by the structure of Formula (2-V): 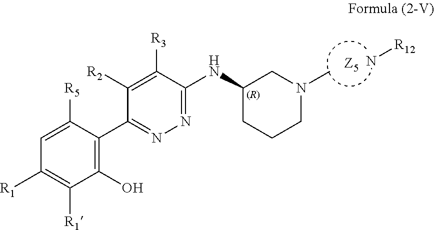
or a pharmaceutically acceptable salt, solvate, isomer, atropisomer or tautomer thereof, wherein:
ring 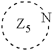
is azetidine, pyrrolidine, piperidine, or azepane;
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, —CF₃;
R₃ is selected from the group consisting of H, C1-C3 alkyl, —CF₃;
or R₂ together with R₃ form a 5- or 6-membered aromatic or non-aromatic ring, all carbon or with O or N within the ring, optionally substituted with -Me or —OH;
R₄ is selected from the group consisting of —H, —F, C1-C3 alkyl, and —CF₃;
R₅ is selected from the group consisting of —H, -Me and —CF₃; and
R₁₂ is selected from the group consisting of: 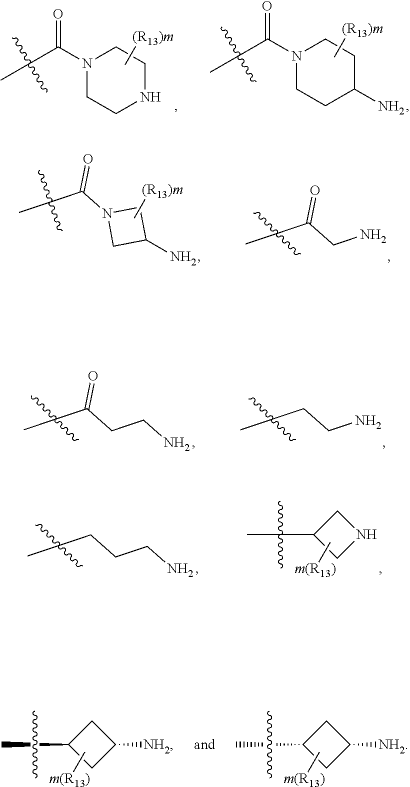
wherein m is 1, 2, or 3 and each R₁₃ is independently selected from the group consisting of —H, C1-C3 alkyl, and —CF; or two R₁₃ groups connect to form a cyclopropyl or cyclobutyl ring.

52. The compound of claim 51 wherein ring 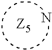
is azetidine or piperidine;

53. The compound of claim 52 wherein R₁ is selected from —F, —CF3; R1′ is —H; R2 is selected from —H, -Me, and —CF3; R₃ is selected from —H, or -Me, or R₂ and R₃ form a 5- or 6-membered aromatic or non-aromatic ring, all carbon or with O within the ring; R₄ is —H or -Me; R₅ is selected from —H and -Me; R₁₂ is selected from any one of the following:

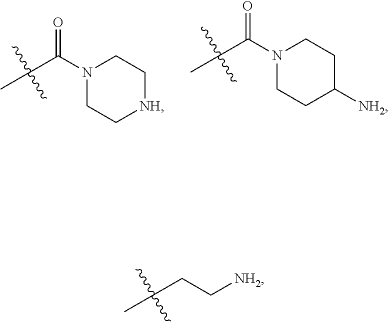

54. The compound of claim 51, further represented by any one of the following:

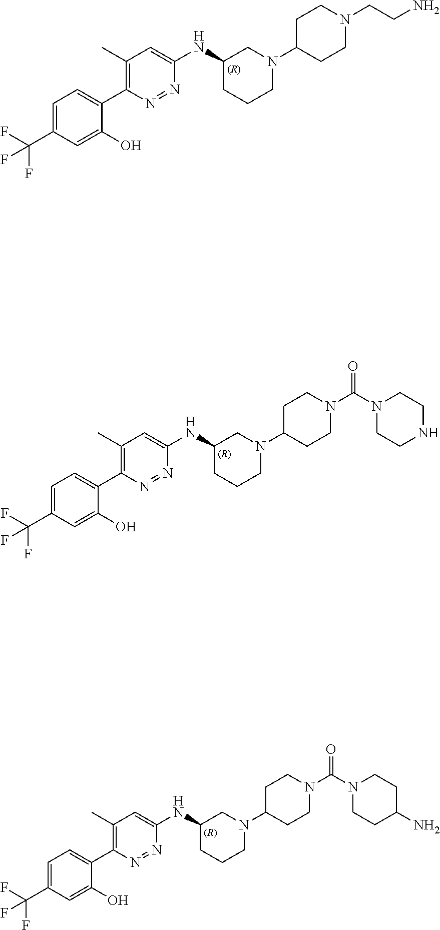

55. A compound represented by the structure of Formula (2-VI): 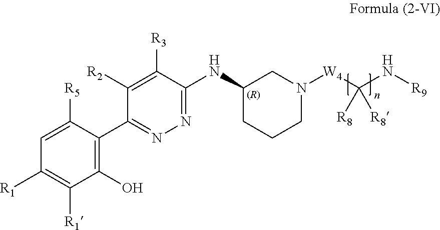
or a pharmaceutically acceptable salt, solvate, hydrate, isomer, atropisomer, or tautomer thereof,
wherein R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, and —CF₃;
R₃ is selected from the group consisting of H, C1-C3 alkyl, and —CF₃;
or R₂ together with R₃ form a 5- or 6-membered aromatic or non-aromatic ring, all carbon or with O or N within the ring, optionally substituted with -Me or —OH;
R₅ is selected from the group consisting of —H, -Me and —CF₃;
W₄ is selected from the group consisting of —CH₂—, —CH₂CH₂—, 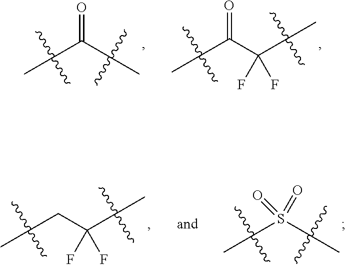
R₈ and R₈′ are independently selected from the group consisting of —H, C1-C5 alkyl, C1-C5 cycloalkyl, and —CF₃; and
R₉ is selected from the group consisting of —H, C1-C6 alkyl, C1-C6 cycloalkyl, heterocycloalkyl,

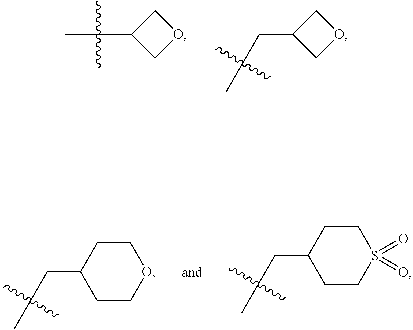 [SMILES: *C1COC1 ; *CC1COC1 ; *CC1CCOCC1 ; *CC1CCS(=O)(=O)CC1]

56. A compound of claim 55 wherein R1 is selected from —F, —CF3; R1′ is —H; R2 is selected from —H, -Me, or —CF3; R3 is selected from —H or -Me, or R₂ and R₃ form a 5- or 6-membered aromatic or non-aromatic ring, all carbon or with O within the ring; R₅ is selected from —H and -Me.

57. A compound of claim 56 wherein W4 is selected from —CH₂— or 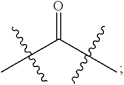 [SMILES: *C(*)=O]
R₈ and R₈′ are —H; n is 1; R₉ is —H.

58. A compound of claim 55-57 wherein R8 and R8′ are —H; n is 1; R9 is selected from

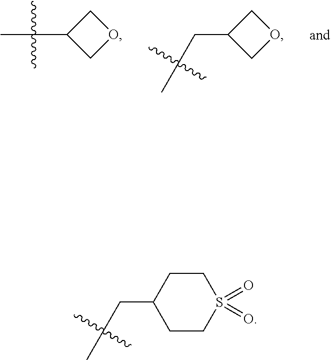 [SMILES: *C1COC1 ; *CC1COC1 ; *CC1CCS(=O)(=O)CC1]

59. The compound of claim 55, further represented by any one of the following:

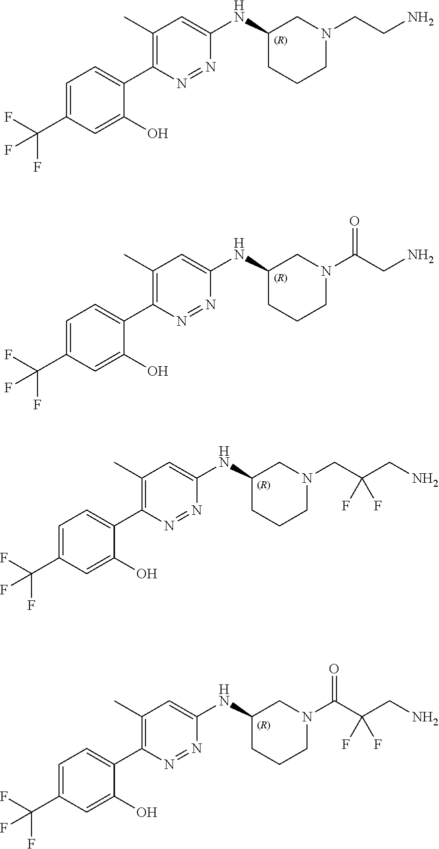

60. The compound of claim 55, further represented by any one of the following:

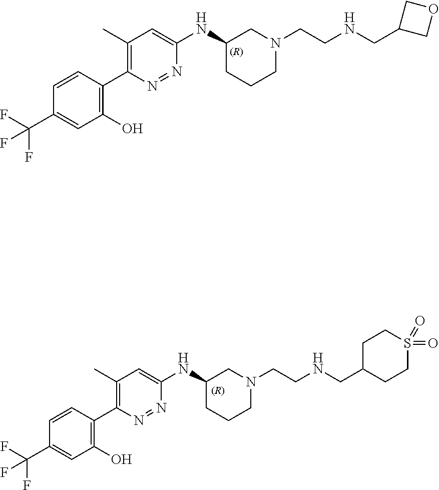

61. A compound represented by the structure of Formula (2-VII): 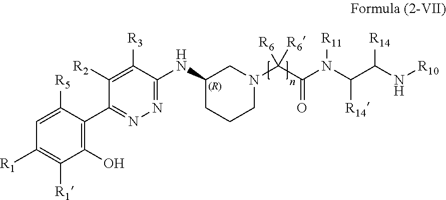
or a pharmaceutically acceptable salt, solvate, hydrate, isomer, atropisomer, or tautomer thereof, wherein:
R₁ is selected from the group consisting of —CN, —H, —F, and —CF₃;
R₁′ is H;
or R₁ and R₁′ together form a thiophene ring;
R₂ is selected from the group consisting of H, C1-C3 alkyl, and —CF₃;
R₃ is selected from the group consisting of H, C1-C3 alkyl, and —CF₃;
or R₂ together with R₃ form a 5- or 6-membered aromatic or non-aromatic ring, all carbon or with O or N within the ring, optionally substituted with -Me or —OH;
R₅ is selected from—the group consisting of H, -Me and —CF₃;
R₆ and R₆′ are independently selected from the group consisting of —H, —F, C1-C6 alkyl, C1-C₆ cycloalkyl, and —CF₃,
or R₆ and R₆′ together with the atom they are bonded to form a C1-C6 cycloalkyl or C1-C6 heterocycloalkyl ring;
n is 1, 2, 3, or 4;
R₁₄ and R₁₄′ are independently selected from the group consisting of H, C1-C5 alkyl (including cycloalkyl), and —CF₃;
or R₁₄ and R₁₄′ together with the atoms that they are bonded to form a optionally substituted C3-C6 cycloalkyl or optionally substituted C3-C6 heterocycloalkyl ring;
R₁₀ is selected from the group consisting of —H, C1-C7 alkyl, C1-C7 cycloalkyl, C1-C7 methylcycloalkyl), 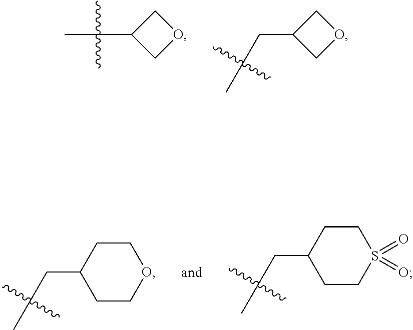 [SMILES: *C1COC1 ; *CC1COC1 ; *CC1CCOCC1 ; *CC1CCS(=O)(=O)CC1]
and
R₁₁ is selected from the group consisting of H, C1-C7 alkyl, C1-C7 cycloalkyl, C1-C7 methylcycloalkyl.

62. The compound of claim 61 wherein R₁ is —F or —CF₃; R₁′ is H; R₂ is —H, -Me, or —CF₃; R₃ is —H or -Me, or R₂ and R₃ form a 5- or 6-membered aromatic or non-aromatic ring, all carbon or with O within the ring; R₅ is selected from —H and -Me; R₆ and R₆′ are —H; n is 1; R₁₄ and R₁₄′ are —H; R₁₀ is —H; R₁₁ is selected from —H or -Me.

63. The compound of claim 61, further represented by the following:

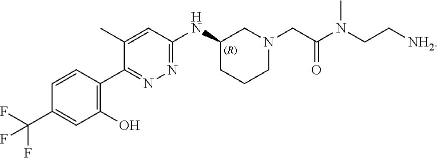

64. A pharmaceutical composition comprising a therapeutically effective amount of a compound of any one of claims 1-63 and a pharmaceutically acceptable excipient.

65. A method of preventing, treating, or ameliorating one or more diseases in a subject, comprising administering a compound of any one of claims 1-63, or a pharmaceutically acceptable salt thereof, to a subject in need thereof.

66. The method of claim 65, wherein the disease is selected from the group consisting of age-related macular degeneration (wet and dry) (AMD), atrophic macular degeneration, bacterial endophthalmitis, bacterial infections of the eye, bacterial corneal ulcers, bacterial keratitis, Behcet's syndrome, cataract, choroidal neovascularization, CMV-retinitis, chronic eye disease, delayed diabetic corneal wound healing and nerve degeneration, diabetic macular edema (DME), diabetic retinopathy (DR), conjunctivitis, corneal allograft rejection, corneal edema, dry eye disease (DED), Graves' disease, Fuchs endothelial corneal dystrophy, fungal endophthalmitis, fungal keratitis, fungal infections of the eye, glaucoma (acute and non-acute), geographic atrophy (GA), inflammatory eye disease, keratitis fugax hereditaria (KFH), LPS-induced ocular inflammation, Mooren's ulcer (MU), neuronal death in retinal ischemia/reperfusion injury, ocular hypertension, ocular inflammation associated with, ocular manifestations of rheumatoid arthritis and systemic lupus erythematosus (SLE), ocular limbal squamous cell carcinoma, optic neuritis, parasitic infections of the eye, pathologic neovascularization, excitatory retinal overstimulation, lipofuscin and A2E-mediated oxidative damage incl, but not limited to ROS/oxidative stress reduction, retinal ganglion cell (RGC) dysfunction and death in response to ocular hypertension (OHT)-induced stress (OHT-glaucoma), retinoblastoma, retinitis, retinal vasculitis, retinal vein occlusion (RVO), Sjörgen's syndrome, sterile corneal inflammation, stroke induced retinal injury in diabetes, traumatic optic neuropathy/trauma (optic nerve crush) incl, but not limited to progressive optic neuropathy, ulcerative keratitis, uveal melanoma, uveitis (anterior/intermediate/posterior, pan-uveitis), and viral infections of the eye.

67. The method of claim 65, wherein the disease is characterized by a disease progression that comprises the activity of IL-1ß, IL-18, or both.

68. The method of any one of claims 65-67, wherein the disease is selected from the group consisting of age-related macular degeneration (wet and dry) (AMD), atrophic macular degeneration, bacterial keratitis, Behcet's syndrome, choroidal neovascularization, chronic eye disease, diabetic macular edema (DME), diabetic retinopathy (DR), dry eye disease (DED, glaucoma (acute and non-acute), geographic atrophy (GA), retinopathy, inflammatory eye disease, ocular inflammation associated with cryopyrin-associated periodic syndrome (CAPS), ocular manifestations of rheumatoid arthritis and systemic lupus erythematosus (SLE), retinitis, retinal vasculitis, retinal vein occlusion (RVO), progressive optic neuropathy, and uveitis (anterior/intermediate/posterior, pan-uveitis).

69. The method of any one of claims 65-68, wherein the disease is at least one chronic inflammatory disorder.

70. The method of claim 69, wherein the disease is characterized by a disease progression pathology that comprises the activity of NLRP3 inflammasome or IL-1ß secretion, IL-18 secretion or both.

71. The method of claim 70, wherein the NLRP3 inflammasome comprises at least one mutation.

72. The method of any one of claims 65-71, wherein the method is a monotherapy.

73. The method of any one of claims 65-71, wherein the method includes administering at least one other form of treatment.

74. The method of any one of claims 65-73, wherein the subject is human.
